(19) United States
(12) Patent Application Publication
(10) Pub. No.: US 2024/0408169 A1
(43) Pub. Date: Dec. 12, 2024

(54) CYCLIN INHIBITORS

(71) Applicant: CIRCLE PHARMA, INC., South San Francisco, CA (US)

(72) Inventors: Andrew T. BOCKUS, South San Francisco, CA (US); Breena F. WALTON, South San Francisco, CA (US); Constantine KREATSOULAS, South San Francisco, CA (US); James B. AGGEN, South San Francisco, CA (US); Justin A. SHAPIRO, South San Francisco, CA (US); Megan DEMART, South San Francisco, CA (US); Nathan J. DUPPER, South San Francisco, CA (US); Sik Fai Siegfried LEUNG, South San Francisco, CA (US); Chinmay BHATT, South San Francisco, CA (US); Samuel METOBO, South San Francisco, CA (US); Kai YANG, South San Francisco, CA (US); Ming Hsun HO, South San Francisco, CA (US); Rajinder SINGH, South San Francisco, CA (US); Ramesh B. BAMBAL, South San Francisco, CA (US)

(21) Appl. No.: 18/667,080

(22) Filed: May 17, 2024

Related U.S. Application Data
(60) Provisional application No. 63/467,651, filed on May 19, 2023.
(60) Provisional application No. 63/612,533, filed on Dec. 20, 2023.
(60) Provisional application No. 63/637,984, filed on Apr. 24, 2024.

(51) Int. Cl. A61K 38/12; C07K 7/64

(52) U.S. Cl. CPC A61K 38/12; C07K 7/64

(57) ABSTRACT

Disclosed herein are compounds of Formula (I), intermediates thereof, and methods for making the same:

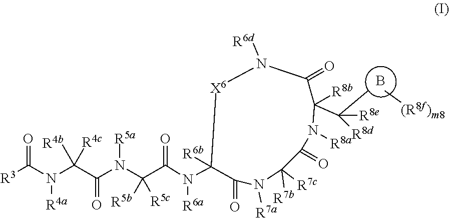

Also described herein are the use of such compounds and compositions for the treatment of diseases and disorders that are mediated, at least in part, by one or more cyclins, including cancer.

### CROSS-REFERENCES TO RELATED APPLICATIONS

This application claims priority to U.S. Provisional Application No. 63/467,651, filed May 19, 2023; U.S. Provisional Application No. 63/612,533, filed Dec. 20, 2023; and U.S. Provisional Application No. 63/637,984, filed Apr. 24, 2024, the disclosure of each is hereby incorporated by reference in their entirety for all purposes.

### BACKGROUND

Cyclins are a family of proteins that play a central role in the regulation of the cell cycle. Specific cyclins, including Cyclins D, E, A and B, are expressed at the different stages of the cell cycle, during which they bind and activate their cognate cyclin dependent kinases (CDKs), including CDKs 1, 2, 4 and 6, to form cyclin-CDK complexes that orchestrate progression and transitions through the different stages of the cell cycle. Disruptions of the normal regulatory functions of cyclin-CDK complexes are common drivers of oncogenesis and the rapid proliferation of cancer cells. The central role of cyclins and CDKs in the cell cycle makes these proteins and their complexes attractive targets for treating proliferative disorders and cancer. To date, most inhibitors of cyclin-CDK complexes target the kinase activity of CDKs (“CDK inhibitors”) and include therapeutics both in development and approved for clinical use. Alternative approaches could include disrupting the association of cyclins with CDKs or the interaction of a particular cyclin-CDK complex with its substrates or regulators.

Although CDK inhibitors have been developed and proven successful in certain cancers, they are currently limited by their relative lack of selectivity, small therapeutic window, and ultimately the development of resistance. As such, there is a need to develop agents that offer alternative approaches to inhibiting the function of cyclin-CDK complexes as a means to modulate the cell cycle. Such agents could provide new tools in the treatment of proliferative diseases. The present disclosure addresses this need by providing compounds that inhibit the binding of substrates to various cyclins, thereby disrupting the function of cyclin-CDK complexes.

### BRIEF SUMMARY

In one embodiment, provided herein is a compound of Formula (I):

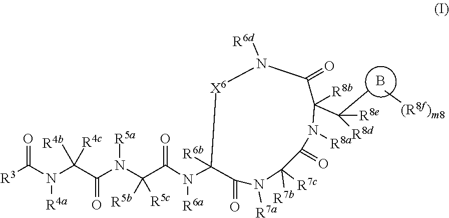

wherein
R³ is
   (a) C₁₋₆ alkyl, C₂₋₆ alkenyl, C₂₋₆ alkynyl, or C₁₋₆ haloalkyl, each substituted with 0 to 5 R^{3a},
   (b) C₃₋₁₂ cycloalkyl substituted with 0 to 5 R^{3b}, or
   (c) heterocycloalkyl having 3 to 12 ring members and 1 to 4 heteroatoms, each independently N, O, or S, wherein the heterocycloalkyl is substituted with 0 to 5 R^{3c};
   (g) heteroaryl having 5 to 6 ring members and 1 to 3 heteroatoms, each independently N, O, or S, wherein the heteroaryl is substituted with 0, 1, 2, 3, 4, or 5 R^{3g};
each R^{3a} is —OH, C₁₋₆ alkoxy, C₁₋₆ haloalkoxy, or —O—C(O)C₁₋₆ alkyl;
each R^{3b} is independently C₁₋₆ alkyl, C₂₋₆ alkenyl, C₂₋₆ alkynyl, —OH, C₁₋₆ alkoxy, halo, C₁₋₆ haloalkyl, C₁₋₆ haloalkoxy, or cyano;
each R^{3c} is independently C₁₋₆ alkyl, halo, C₁₋₆ haloalkyl, cyano, C₃₋₆ cycloalkyl, or oxo;
each R^{3g} is independently C₁₋₆ alkyl, halo, C₁₋₆ haloalkyl, or C₃₋₆ cycloalkyl;
R^{4a} is H or C₁₋₆ alkyl;
R^{4b} and R^{4c} are each independently H, C₁₋₆ alkyl, C₁₋₆ haloalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S;
alternatively, R^{4c} and R^{4a} together with the carbon and nitrogen to which each is attached combine to form a heterocycloalkyl having 3 to 6 ring members and 1 to 3 heteroatoms, each independently N, O, or S, wherein the heterocycloalkyl is substituted with 0 to 4 R^{4a1};
each R^{4a1} is independently C₁₋₆ alkyl, —OH, C₁₋₆ alkyl-OH, C₁₋₆ alkoxy, or halo;
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, —C₁₋₆ alkyl-C(O)NR^{5b1}R^{5b2}, —C₁₋₆ alkyl-C(O)OR^{5b1}, C₃₋₆ cycloalkyl, C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or
—C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S, and wherein each cycloalkyl, heterocycloalkyl, phenyl, and heteroaryl is substituted with 0 to 3 R^{5b5};
each R^{5b1} and R^{5b2} are independently H, C₁₋₄ alkyl, or C₁₋₄ haloalkyl;
alternatively, R^{5b1} and R^{5b2} together with the nitrogen to which each is attached combine to form a heterocycloalkyl having 3 to 6 ring members and 0 to 1 additional heteroatoms, wherein the heterocycloalkyl is substituted with 0 to 2 R^{5b3};
each R^{5b3} and R^{5b5} is independently C₁₋₆ alkyl, halo, C₁₋₆ haloalkyl, or cyano;
X⁶ is C₆₋₇ alkylene or C₆₋₇ alkenylene;
R^{6a} is H, C₁₋₆ alkyl, C₁₋₆ deuteroalkyl, C₂₋₆ alkoxyalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein the heterocycloalkyl has 3 to 6 ring members and 1 to 3 heteroatoms, each independently N, O, or S, and the heteroaryl has 5 to 6 ring members and 1 to 3 heteroatoms, each independently N, O, or S;
R^{6b} and R^{6d} are each independently H or C₁₋₆ alkyl;
R^{7a} is H or C₁₋₆ alkyl;
R^{7b} and R^{7c} are each independently H, C₁₋₆ alkyl, C₂₋₆ alkenyl, C₁₋₆ haloalkyl, C₁₋₆ alkyl-OH, C₃₋₆ cycloalkyl, or —C₁₋₆ alkyl-C₃₋₆ cycloalkyl;
R^{8a} is H, C₁₋₆ alkyl, C₂₋₆ alkoxyalkyl, C₃₋₆ cycloalkyl or —C₁₋₆ alkyl-C₃₋₆ cycloalkyl;
R^{8b}, R^{8d}, and R^{8e} are each independently H or C₁₋₆ alkyl;
ring B is C₆₋₁₂ aryl or heteroaryl having 5 to 12 ring members and 1 to 6 heteroatoms, each independently N, O or S;
the subscript m8 is an integer from 0 to 5;
each R^{8f} is independently C₁₋₆ alkyl, C₂₋₆ alkenyl, C₂₋₆ alkynyl, C₁₋₆ alkoxy, C₁₋₆ deuteroalkoxy, halo, C₁₋₆ haloalkyl, cyano, —X^{8f}-cyano, C₃₋₁₂ cycloalkyl,
—X^{8f}—C₃₋₆ cycloalkyl, —CH═CR^{8f1}R^{8f2}, heterocycloalkyl,
—X^{8f}-heterocycloalkyl, C₆₋₁₂ aryl, —X^{8f}—C₆₋₁₂ aryl, heteroaryl, or
—X^{8f}-heteroaryl, wherein each heterocycloalkyl has 3 to 12 ring members and 1 to 4 heteroatoms, each independently N, O, or S, and each heteroaryl has 5 to 10 ring members and 1 to 4 heteroatoms, each independently N, O, or S, wherein each alkynyl, cycloalkyl, heterocycloalkyl, aryl, and heteroaryl is substituted with 0 to 3 R^{8f3};
alternatively, two R^{8f} groups on adjacent ring vertices combine to form a C₃₋₆ cycloalkyl or a heterocycloalkyl having 3 to 6 ring members and 1 to 3 heteroatoms, each independently N, O or S, wherein the cycloalkyl or heterocycloalkyl is substituted with 0 to 3 R^{8f3};
each X^{8f} is independently C₁₋₆ alkylene, C₂₋₆ alkenylene, —O—C₁₋₆ alkylene, C(O), O, or S;
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3};
each R^{8f3} is independently C₁₋₆ alkyl, —Y⁸—C₁₋₆ alkyl, C₁₋₆ deuteroalkyl,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, —(C₁₋₂ alkyl-O)₁₋₄—C₁₋₂ alkyl, C₁₋₆ alkoxy, halo, C₁₋₆ haloalkyl, —Y⁸—C₁₋₆ haloalkyl, cyano, —C₁₋₆ alkyl-cyano, —C₁₋₆ alkyl-NR^{8g}R^{8h}, oxo, C₃₋₆ cycloalkyl, —X^{8f3}—C₃₋₆ cycloalkyl, heterocycloalkyl, —X^{8f3}-heterocycloalkyl, phenyl, —X^{8f3}-phenyl, heteroaryl, or —X^{8f3}-heteroaryl, wherein each heterocycloalkyl has 3 to 12 members and 1 to 4 heteroatoms, each independently N, O, S, or S(O)₂, and each heteroaryl has 5 to 10 ring members and 1 to 4 heteroatoms, each independently N, O, or S, and wherein each heterocycloalkyl and heteroaryl are substituted with 0 to 2 C₁₋₄ alkyl or halo;
each X^{8f3} is independently C₁₋₆ alkylene, O, C(O), or S(O)₂;
each Y⁸ is independently C(O), C(O)O, N(R^{8f4})C(O), O, S, or S(O)₂;
each R^{8g} and R^{8h} is independently H, C₁₋₃ alkyl, or C₁₋₆ haloalkyl;
alternatively, two R^{8f3} groups on the same or adjacent ring vertices combine to form a C₃₋₆ cycloalkyl or a heterocycloalkyl having 3 to 6 ring members and 1 to 3 heteroatoms, each independently N, O or S, wherein nitrogen atom ring members in the heterocycloalkyl are substituted with 0 to 1 C₁₋₄ alkyl; and
each R^{8f4} is independently H or C₁₋₆ alkyl;
   or a pharmaceutically acceptable salt thereof.

In another embodiment, the present disclosure provides a pharmaceutical composition comprising a compound of the present disclosure, and a pharmaceutically acceptable excipient.

In another embodiment, the present disclosure provides a method of treating a disease or disorder mediated at least in part by cyclin activity, the method comprising administering to a subject in need thereof, a therapeutically effective amount of a compound of the present disclosure, or a pharmaceutically acceptable salt thereof, or a pharmaceutical composition of the present disclosure, thereby treating the disorder or condition.

In another embodiment, the present disclosure provides a method of treating a cancer mediated at least in part by cyclin A, the method comprising administering to a subject in need thereof, a therapeutically effective amount of a compound of the present disclosure, or a pharmaceutically acceptable salt thereof, or a pharmaceutical composition of the present disclosure, thereby treating the cancer.

In another embodiment, the present invention provides intermediates useful in the preparation of compounds of Formula (I).

Other objects, features, and advantages of the present disclosure will be apparent to one of skill in the art from the following detailed description and FIGURES.

### BRIEF DESCRIPTION OF THE DRAWINGS

NOT APPLICABLE

### DETAILED DESCRIPTION

### I. General

Provided herein are compounds and compositions that disrupt the typical cellular function of cyclins. Also provided herein are, for example, methods of treating or preventing a disease, disorder or condition, or a symptom thereof, mediated by cyclin activity.

Complexes between cyclins and cyclin dependent kinases (CDKs) are responsible for phosphorylating a wide range of substrates, thereby modulating the activity of the substrates. Many of these substrates are important in the cell cycle and the cyclin and CDKs that regulate these substrates therefore play key roles in regulating the cell cycle, including Cyclins D, A, E and B, and CDKs 1, 2, 4 and 6. Without being bound to any particular theory, certain substrates, including p21, p27, Rb, E2F and CDC6, first bind to the cyclin-CDK complex via a conserved R×L motif within the substrate (Adams et al. Mol Cell Biol. 1996. 16(12):6223-33.) and bind to a region with the cyclin that is referred to as an R×L binding domain or a “hydrophobic patch” (Brown et al. Nat Cell Biol. 1999.1(7):438-43) and contains a highly conserved MRAIL motif. Compounds that disrupt the binding of substrates to cyclins have been posited to be of potential therapeutic utility, including in the disruption of cancer cell proliferation (Chen et al. Proc Natl Acad Sci USA. 1999. 96(8):4325-9).

Without being bound to any particular theory, it is believed that compounds of the present disclosure inhibit the binding of substrates to the hydrophobic patch region of cyclins including, but not limited to, Cyclins A, E and B. Compounds of the present disclosure include compounds that bind more potently to one or more cyclins.

### II. Definitions

As used herein, the term “about” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 20 carbons atoms, such as, but not limited to heptyl, octyl, nonyl, decyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ—, where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl(ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkenylene” refers to a straight or branched hydrocarbon having at least 2 carbon atoms, one double bond, and linking at least two other groups, i.e., a divalent hydrocarbon radical. Alkenylene can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₃₋₆, C₃₋₇, C₄, C₄₋₅, C₄₋₆, C₄₋₇, C₆₋₇, C₅, C₆ and C₇. The two moieties linked to the alkenylene can be linked to the same atom or different atoms of the alkenylene group. Representative alkenylene groups include, but are not limited to, (E)-hex-2-enylene, (Z)-hex-2-enylene, (E)-hept-2-enylene, (Z)-hept-2-enylene, (E)-hept-3-enylene, and (Z)-hept-3-enylene. Alkenylene moieties can be in the E or Z isomer. Alkenylene groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be substituted or unsubstituted.

“Alkoxyalkyl” refers to alkyl group connected to an oxygen atom that is further connected to an second alkyl group, the second alkyl group being the point of attachment to the remainder of the molecule: alkyl-O-alkyl. The alkyl portion can have any suitable number of carbon atoms, such as C₂₋₆. Alkoxyalkyl groups include, for example, methoxymethyl, ethoxymethyl, methoxyethyl, ethoxyethyl, etc. The alkoxy groups can be substituted or unsubstituted.

“Halo” or “halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, flouromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Cycloalkyl” refers to a saturated or partially unsaturated, monocyclic, spirocyclic, fused or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl. Saturated bicyclic and polycyclic cycloalkyl rings include, for example, norbornane, [2.2.2]bicyclooctane, decahydronaphthalene and adamantane. Cycloalkyl groups can also be partially unsaturated, having one or more double or triple bonds in the ring, but cycloalkyl groups are not aromatic. Representative cycloalkyl groups that are partially unsaturated include, but are not limited to, cyclobuteneyl, cyclopenteneyl, cyclohexeneyl, cyclohexadieneyl (1,3- and 1,4-isomers), cyclohepteneyl, cycloheptadieneyl, cycloocteneyl, cyclooctadieneyl (1,3-, 1,4- and 1,5-isomers), norborneneyl, and norbornadieneyl. When cycloalkyl is a C₃₋₆ monocyclic cycloalkyl, exemplary groups include, but are not limited to cyclopropyl, cyclobutyl, cyclopentyl, cyclohexyl, cyclohexeneyl, cyclohexadieneyl (1,3- and 1,4-isomers). When cycloalkyl is a C₅₋₁₀ fused bicyclic cycloalkyl, exemplary groups include, but are not limited to bicyclo[3.1.0]hexanyl, bicyclo[4.1.0]heptanyl, bicyclo[4.2.0]octanyl, and octahydro-1H-indenyl. When cycloalkyl is a C₅₋₁₀ bridged polycyclic cycloalkyl, exemplary groups include, but are not limited to bicyclo[2.2.1]heptane, bicyclo[3.1.1]heptane, and bicyclo[2.1.1]hexane. When cycloalkyl is a C₅₋₁₀ spirocycloalkyl, exemplary groups include, but are not limited to spiro[3.3]heptane, spiro[3.4]octane, spiro[3.5]nonanyl, spiro[2.5]octane, and spiro[2.4]heptane. Cycloalkyl groups can be substituted or unsubstituted.

“Heterocycloalkyl” refers to a saturated or partially unsaturated, monocyclic, spirocyclic, fused or bridged polycyclic ring assembly having from 3 to 12 ring members and from 1 to 4 heteroatoms of N, O and S. The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), tetrahydropyridine, oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. Heterocycloalkyl groups can be unsubstituted or substituted.

The heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

When heterocycloalkyl is a monocyclic heterocycloalkyl having 3 to 6 ring members and 1 to 3 heteroatoms, representative members include, but are not limited to, pyrrolidine, piperidine, tetrahydrofuran, oxane, tetrahydrothiophene, thiane, pyrazolidine, imidazolidine, piperazine, oxazolidine, isoxzoalidine, thiazolidine, isothiazolidine, morpholine, thiomorpholine, dioxane and dithiane. Heterocycloalkyl can also be monocyclic heterocycloalkyl having 5 to 6 ring members and 1 to 2 heteroatoms, with representative members including, but not limited to, pyrrolidine, piperidine, tetrahydrofuran, tetrahydrothiophene, pyrazolidine, imidazolidine, piperazine, oxazolidine, isoxazolidine, thiazolidine, isothiazolidine, and morpholine.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Heteroaryl” refers to a monocyclic or fused bicyclic or tricyclic aromatic ring assembly containing 5 to 12 ring atoms, where from 1 to 6 of the ring atoms are a heteroatom such as N, O or S. The heteroatoms can also be oxidized, such as, but not limited to, —S(O)— and —S(O)₂—. Heteroaryl groups can include any number of ring atoms, such as, 5 to 6, 5 to 8, 5 to 9, 5 to 10, 5 to 12, or 9 to 12 ring members. Any suitable number of heteroatoms can be included in the heteroaryl groups, such as 1, 2, 3, 4, 5, or 6, or 1 to 2, 1 to 3, 1 to 4, 1 to 5, 2 to 3, 2 to 4, 2 to 5, 2 to 6, 3 to 4, 3 to 5, or 3 to 6. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

The heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

Some heteroaryl groups include those having from 5 to 10 ring members and from 1 to 3 ring atoms including N, O or S, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, isoxazole, indole, isoindole, quinoline, isoquinoline, quinoxaline, quinazoline, phthalazine, cinnoline, benzothiophene, and benzofuran. Other heteroaryl groups include those having from 5 to 8 ring members and from 1 to 3 heteroatoms, such as pyrrole, pyridine, imidazole, pyrazole, triazole, pyrazine, pyrimidine, pyridazine, triazine (1,2,3-, 1,2,4- and 1,3,5-isomers), thiophene, furan, thiazole, isothiazole, oxazole, and isoxazole. Some other heteroaryl groups include those having from 9 to 12 ring members and from 1 to 3 heteroatoms, such as indole, isoindole, quinoline, isoquinoline, quinoxaline, quinazoline, phthalazine, cinnoline, benzothiophene, benzofuran and bipyridine. Still other heteroaryl groups include those having from 5 to 6 ring members and from 1 to 2 ring atoms including N, O or S, such as pyrrole, pyridine, imidazole, pyrazole, pyrazine, pyrimidine, pyridazine, thiophene, furan, thiazole, isothiazole, oxazole, and isoxazole.

“Thiophenyl” refers to a thiophene radical.

As used herein, the term “oxo” refers to an oxygen atom connected to the point of attachment by a double bond (═O).

“Pharmaceutically acceptable excipient” refers to a substance that aids the formulation and/or administration of an active agent to a subject. Pharmaceutical excipients useful in the present disclosure include, but are not limited to, binders, fillers, disintegrants, lubricants, coatings, sweeteners, flavors and colors. One of skill in the art will recognize that other pharmaceutical excipients are useful in the present disclosure.

“Subject” refers to animals such as mammals, including, but not limited to, primates (e.g., humans), cows, sheep, goats, horses, dogs, cats, rabbits, rats, mice and the like. In some embodiments, the subject is a human.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins)

“Treat”, “treating” and “treatment” refers to any indicia of success in the treatment or amelioration of an injury, pathology, condition, or symptom (e.g., pain), including any objective or subjective parameter such as abatement; remission; diminishing of symptoms or making the symptom, injury, pathology or condition more tolerable to the patient; decreasing the frequency or duration of the symptom or condition. The treatment or amelioration of symptoms can be based on any objective or subjective parameter; including, e.g., the result of a physical examination.

### III. Compounds

In some embodiments, the present disclosure provides a compound of Formula (I)

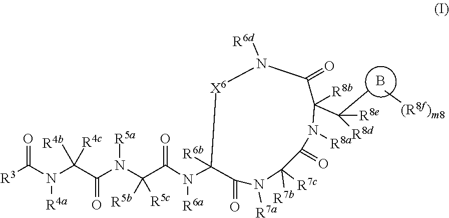

R³ is
   (a) C₁₋₆ alkyl, C₂₋₆ alkenyl, C₂₋₆ alkynyl, or C₁₋₆ haloalkyl, each substituted with 0 to 5 R^{3a},
   (b) C₃₋₁₂ cycloalkyl substituted with 0 to 5 R^{3b}, or
   (c) heterocycloalkyl having 3 to 12 ring members and 1 to 4 heteroatoms, each independently N, O, or S, wherein the heterocycloalkyl is substituted with 0 to 5 R^{3c};
   (g) heteroaryl having 5 to 6 ring members and 1 to 3 heteroatoms, each independently N, O, or S, wherein the heteroaryl is substituted with 0, 1, 2, 3, 4, or 5 R^{3g};
each R^{3a} is —OH, C₁₋₆ alkoxy, C₁₋₆ haloalkoxy, or —O—C(O)C₁₋₆ alkyl;
each R^{3b} is independently C₁₋₆ alkyl, C₂₋₆ alkenyl, C₂₋₆ alkynyl, —OH, C₁₋₆ alkoxy, halo, C₁₋₆ haloalkyl, C₁₋₆ haloalkoxy, or cyano;
each R^{3c} is independently C₁₋₆ alkyl, halo, C₁₋₆ haloalkyl, cyano, C₃₋₆ cycloalkyl, or oxo;
each R^{3g} is independently C₁₋₆ alkyl, halo, C₁₋₆ haloalkyl, or C₃₋₆ cycloalkyl;
R^{4a} is H or C₁₋₆ alkyl;
R^{4b} and R^{4c} are each independently H, C₁₋₆ alkyl, C₁₋₆ haloalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S;
alternatively, R^{4c} and R^{4a} together with the carbon and nitrogen to which each is attached combine to form a heterocycloalkyl having 3 to 6 ring members and 1 to 3 heteroatoms, each independently N, O, or S, wherein the heterocycloalkyl is substituted with 0 to 4 R^{4a1};
each R^{4a1} is independently C₁₋₆ alkyl, —OH, C₁₋₆ alkyl-OH, C₁₋₆ alkoxy, or halo;
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, —C₁₋₆ alkyl-C(O)NR^{5b1}R^{5b2}, —C₁₋₆ alkyl-C(O)OR^{5b1}, C₃₋₆ cycloalkyl, C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or
—C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S, and wherein each cycloalkyl, heterocycloalkyl, phenyl, and heteroaryl is substituted with 0 to 3 R^{5b5};
each R^{5b1} and R^{5b2} are independently H, C₁₋₄ alkyl, or C₁₋₄ haloalkyl; alternatively, R^{5b1} and R^{5b2} together with the nitrogen to which each is attached combine to form a heterocycloalkyl having 3 to 6 ring members and 0 to 1 additional heteroatoms, wherein the heterocycloalkyl is substituted with 0 to 2 R^{5b3};
each R^{5b3} and R^{5b5} is independently C₁₋₆ alkyl, halo, C₁₋₆ haloalkyl, or cyano;
X⁶ is C₆₋₇ alkylene or C₆₋₇ alkenylene;
R^{6a} is H, C₁₋₆ alkyl, C₁₋₆ deuteroalkyl, C₂₋₆ alkoxyalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein the heterocycloalkyl has 3 to 6 ring members and 1 to 3 heteroatoms, each independently N, O, or S, and the heteroaryl has 5 to 6 ring members and 1 to 3 heteroatoms, each independently N, O, or S;
R^{6b} and R^{6d} are each independently H or C₁₋₆ alkyl;
R^{7a} is H or C₁₋₆ alkyl;
R^{7b} and R^{7c} are each independently H, C₁₋₆ alkyl, C₂₋₆ alkenyl, C₁₋₆ haloalkyl, C₁₋₆ alkyl-OH, C₃₋₆ cycloalkyl, or —C₁₋₆ alkyl-C₃₋₆ cycloalkyl;
R^{8a} is H, C₁₋₆ alkyl, C₂₋₆ alkoxyalkyl, C₃₋₆ cycloalkyl or —C₁₋₆ alkyl-C₃₋₆ cycloalkyl;
R^{8b}, R^{8d}, and R^{8e} are each independently H or C₁₋₆ alkyl;
ring B is C₆₋₁₂ aryl or heteroaryl having 5 to 12 ring members and 1 to 6 heteroatoms, each independently N, O or S;
the subscript m8 is an integer from 0 to 5;
each R^{8f} is independently C₁₋₆ alkyl, C₂₋₆ alkenyl, C₂₋₆ alkynyl, C₁₋₆ alkoxy, C₁₋₆ deuteroalkoxy, halo, C₁₋₆ haloalkyl, cyano, —X^{8f}-cyano, C₃₋₁₂ cycloalkyl,
—X^{8f}—C₃₋₆ cycloalkyl, —CH═CR^{8f1}R^{8f2}, heterocycloalkyl,
—X^{8f}-heterocycloalkyl, C₆₋₁₂ aryl, —X^{8f}—C₆₋₁₂ aryl, heteroaryl, or
—X^{8f}-heteroaryl, wherein each heterocycloalkyl has 3 to 12 ring members and 1 to 4 heteroatoms, each independently N, O, or S, and each heteroaryl has 5 to 10 ring members and 1 to 4 heteroatoms, each independently N, O, or S, wherein each alkynyl, cycloalkyl, heterocycloalkyl, aryl, and heteroaryl is substituted with 0 to 3 R^{8f3};
alternatively, two R^{8f} groups on adjacent ring vertices combine to form a C₃₋₆ cycloalkyl or a heterocycloalkyl having 3 to 6 ring members and 1 to 3 heteroatoms, each independently N, O or S, wherein the cycloalkyl or heterocycloalkyl is substituted with 0 to 3 R^{8f3};
each X^{8f} is independently C₁₋₆ alkylene, C₂₋₆ alkenylene, —O—C₁₋₆ alkylene, C(O), O, or S;
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3};
each R^{8f3} is independently C₁₋₆ alkyl, —Y⁸—C₁₋₆ alkyl, C₁₋₆ deuteroalkyl,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, —(C₁₋₂ alkyl-O)₁₋₄—C₁₋₂ alkyl, C₁₋₆ alkoxy, halo, C₁₋₆ haloalkyl, —Y⁸—C₁₋₆ haloalkyl, cyano, —C₁₋₆ alkyl-cyano, —C₁₋₆ alkyl-NR^{8g}R^{8h}, oxo, C₃₋₆ cycloalkyl, —X^{8f3}—C₃₋₆ cycloalkyl, heterocycloalkyl, —X^{8f3}-heterocycloalkyl, phenyl, —X^{8f3}-phenyl, heteroaryl, or —X^{8f3}-heteroaryl, wherein each heterocycloalkyl has 3 to 12 members and 1 to 4 heteroatoms, each independently N, O, S, or S(O)₂, and each heteroaryl has 5 to 10 ring members and 1 to 4 heteroatoms, each independently N, O, or S, and wherein each heterocycloalkyl and heteroaryl are substituted with 0 to 2 C₁₋₄ alkyl or halo;
each X^{8f3} is independently C₁₋₆ alkylene, O, C(O), or S(O)₂;
each Y⁸ is independently C(O), C(O)O, N(R^{8f4})C(O), O, S, or S(O)₂;
each R^{8g} and R^{8h} is independently H, C₁₋₃ alkyl, or C₁₋₆ haloalkyl;
alternatively, two R^{8f3} groups on the same or adjacent ring vertices combine to form a C₃₋₆ cycloalkyl or a heterocycloalkyl having 3 to 6 ring members and 1 to 3 heteroatoms, each independently N, O or S, wherein nitrogen atom ring members in the heterocycloalkyl are substituted with 0 to 1 C₁₋₄ alkyl; and
each R^{8f4} is independently H or C₁₋₆ alkyl;
or a pharmaceutically acceptable salt thereof.

In some embodiments, the present disclosure provides a compound of Formula (I)

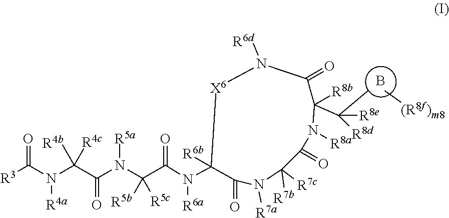

wherein
R³ is
   (a) C₁₋₆ alkyl, C₂₋₆ alkenyl, C₂₋₆ alkynyl, or C₁₋₆ haloalkyl, each substituted with 0 to 5 R^{3a},
   (b) C₃₋₁₂ cycloalkyl substituted with 0 to 5 R^{3b}, or
   (c) heterocycloalkyl having 3 to 12 ring members and 1 to 4 heteroatoms, each independently N, O, or S, wherein the heterocycloalkyl is substituted with 0 to 5 R^{3c};
each R^{3a} is —OH, C₁₋₆ alkoxy, C₁₋₆ haloalkoxy, or —O—C(O)C₁₋₆ alkyl;
each R^{3b} is independently C₁₋₆ alkyl, C₂₋₆ alkenyl, C₂₋₆ alkynyl, —OH, C₁₋₆ alkoxy, halo, C₁₋₆ haloalkyl, C₁₋₆ haloalkoxy, or cyano;
each R^{3c} is independently C₁₋₆ alkyl, C₁₋₆ haloalkyl, C₃₋₆ cycloalkyl, or oxo;
R^{4a} is H or C₁₋₆ alkyl;
R^{4b} and R^{4c} are each independently H, C₁₋₆ alkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S;
alternatively, R^{4c} and R^{4a} together with the carbon and nitrogen to which each is attached combine to form a heterocycloalkyl having 3 to 6 ring members, wherein the heterocycloalkyl is substituted with 0 to 4 R^{4a1};
each R^{4a1} is independently C₁₋₆ alkyl, —OH, C₁₋₆ alkyl-OH, C₁₋₆ alkoxy, or halo;
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, C₃₋₆ cycloalkyl, C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or
—C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S, and wherein each cycloalkyl, heterocycloalkyl, phenyl, and heteroaryl is substituted with 0 to 3 R^{5b5};
each R^{5b5} is independently C₁₋₆ alkyl, halo, or C₁₋₆ haloalkyl;
X⁶ is C₆₋₇ alkylene or C₆₋₇ alkenylene;
R^{6a} is H, C₁₋₆ alkyl, C₂₋₆ alkoxyalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein the heterocycloalkyl has 3 to 6 ring members and 1 to 3 heteroatoms, each independently N, O, or S, and the heteroaryl has 5 to 6 ring members and 1 to 3 heteroatoms, each independently N, O, or S;
R^{6b} and R^{6d} are each independently H or C₁₋₆ alkyl;
R^{7a} is H or C₁₋₆ alkyl;
R^{7b} and R^{7c} are each independently H, C₁₋₆ alkyl, C₂₋₆ alkenyl, C₁₋₆ haloalkyl, C₁₋₆ alkyl-OH, C₃₋₆ cycloalkyl, or —C₁₋₆ alkyl-C₃₋₆ cycloalkyl;
R^{8a} is H, C₁₋₆ alkyl, C₂₋₆ alkoxyalkyl, C₃₋₆ cycloalkyl or —C₁₋₆ alkyl-C₃₋₆ cycloalkyl;
R^{8b}, R^{8d}, and R^{8e} are each independently H or C₁₋₆ alkyl;
ring B is C₆₋₁₂ aryl or heteroaryl having 5 to 12 ring members and 1 to 6 heteroatoms, each independently N, O or S;
the subscript m8 is an integer from 0 to 5;
each R^{8f} is independently C₁₋₆ alkyl, C₂₋₆ alkenyl, C₂₋₆ alkynyl, C₁₋₆ alkoxy, C₁₋₆ deuteroalkoxy, halo, C₁₋₆ haloalkyl, cyano, —X^{8f}-cyano, C₃₋₆ cycloalkyl,
—X^{8f}—C₃₋₆ cycloalkyl, —CH═CR^{8f1}R^{8f2}, heterocycloalkyl,
—X^{8f}-heterocycloalkyl, C₆₋₁₂ aryl, —X^{8f}—C₆₋₁₂ aryl, heteroaryl, or
—X^{8f}-heteroaryl, wherein each heterocycloalkyl has 3 to 12 ring members and 1 to 4 heteroatoms, each independently N, O, or S, and each heteroaryl has 5 to 10 ring members and 1 to 4 heteroatoms, each independently N, O, or S, wherein each alkynyl, cycloalkyl, heterocycloalkyl, aryl, and heteroaryl is substituted with 0 to 3 R^{8f3};
alternatively, two R^{8f} groups on adjacent ring vertices combine to form a C₃₋₆ cycloalkyl or a heterocycloalkyl having 3 to 6 ring members and 1 to 3 heteroatoms, each independently N, O or S, wherein the cycloalkyl or heterocycloalkyl is substituted with 0 to 3 R^{8f3};
each X^{8f} is independently C₁₋₆ alkylene, C₂₋₆ alkenylene, —O—C₁₋₆ alkylene, O, or S;
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3};
each R^{8f3} is independently C₁₋₆ alkyl, —Y⁸—C₁₋₆ alkyl, C₁₋₆ deuteroalkyl,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, —(C₁₋₂ alkyl-O)₁₋₄—C₁₋₂ alkyl, C₁₋₆ alkoxy, halo, C₁₋₆ haloalkyl, —Y⁸—C₁₋₆ haloalkyl, cyano, —C₁₋₆ alkyl-cyano, oxo, C₃₋₆ cycloalkyl, —X^{8f3}—C₃₋₆ cycloalkyl, heterocycloalkyl, —X^{8f3}-heterocycloalkyl, phenyl, —X^{8f3}-phenyl, heteroaryl, or —X^{8f3}-heteroaryl, wherein each heterocycloalkyl has 3 to 12 members and 1 to 4 heteroatoms, each independently N, O, S, or S(O)₂, and each heteroaryl has 5 to 10 ring members and 1 to 4 heteroatoms, each independently N, O, or S, and wherein nitrogen atom ring members in each heterocycloalkyl and heteroaryl are substituted with 0 to 1 C₁₋₄ alkyl;
each X^{8f3} is independently C₁₋₆ alkylene, O, C(O), or S(O)₂;
each Y⁸ is independently C(O), C(O)O, N(R^{8f4})C(O), O, S, or S(O)₂;
alternatively, two R^{8f3} groups on the same or adjacent ring vertices combine to form a C₃₋₆ cycloalkyl or a heterocycloalkyl having 3 to 6 ring members and 1 to 3 heteroatoms, each independently N, O or S, wherein nitrogen atom ring members in the heterocycloalkyl are substituted with 0 to 1 C₁₋₄ alkyl; and
each R^{8f4} is independently H or C₁₋₆ alkyl;
   or a pharmaceutically acceptable salt thereof.

In some embodiments, the present disclosure provides a compound of Formula (I)

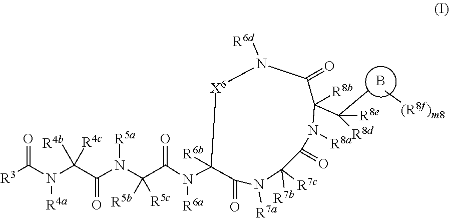

wherein
R³ is
   (a) C₁₋₆ alkyl, C₂₋₆ alkenyl, C₂₋₆ alkynyl, or C₁₋₆ haloalkyl, each substituted with 0 to 5 R^{3a},
   (b) C₃₋₁₂ cycloalkyl substituted with 0 to 5 R^{3b}, or
   (c) heterocycloalkyl having 3 to 12 ring members and 1 to 4 heteroatoms, each independently N, O, or S, wherein the heterocycloalkyl is substituted with 0 to 5 R^{3c};
each R^{3a} is —OH, C₁₋₆ alkoxy, C₁₋₆ haloalkoxy, or —O—C(O)C₁₋₆ alkyl;
each R^{3b} is independently C₁₋₆ alkyl, C₂₋₆ alkenyl, C₂₋₆ alkynyl, —OH, C₁₋₆ alkoxy, halo, C₁₋₆ haloalkyl, C₁₋₆ haloalkoxy, or cyano;
each R^{3c} is independently C₁₋₆ alkyl, C₁₋₆ haloalkyl, C₃₋₆ cycloalkyl, or oxo;
R^{4a} is H or C₁₋₆ alkyl;
R^{4b} and R^{4c} are each independently H, C₁₋₆ alkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S;
alternatively, R^{4c} and R^{4a} together with the carbon and nitrogen to which each is attached combine to form a heterocycloalkyl having 3 to 6 ring members, wherein the heterocycloalkyl is substituted with 0 to 4 R^{4a1};
each R^{4a1} is independently C₁₋₆ alkyl, —OH, C₁₋₆ alkyl-OH, C₁₋₆ alkoxy, or halo;
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, C₃₋₆ cycloalkyl, C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or
—C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S, and wherein each cycloalkyl, heterocycloalkyl, phenyl, and heteroaryl is substituted with 0 to 3 R^{5b5};
each R^{5b5} is independently C₁₋₆ alkyl, halo, or C₁₋₆ haloalkyl;
X⁶ is C₆₋₇ alkylene or C₆₋₇ alkenylene;
R^{6a} is H, C₁₋₆ alkyl, C₂₋₆ alkoxyalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein the heterocycloalkyl has 3 to 6 ring members and 1 to 3 heteroatoms, each independently N, O, or S, and the heteroaryl has 5 to 6 ring members and 1 to 3 heteroatoms, each independently N, O, or S;
R^{6b} and R^{6d} are each independently H or C₁₋₆ alkyl;
R^{7a} is H or C₁₋₆ alkyl;
R^{7b} and R^{7c} are each independently H, C₁₋₆ alkyl, C₂₋₆ alkenyl, C₁₋₆ haloalkyl, C₁₋₆ alkyl-OH, C₃₋₆ cycloalkyl, or —C₁₋₆ alkyl-C₃₋₆ cycloalkyl;
R^{8a} is H, C₁₋₆ alkyl, C₂₋₆ alkoxyalkyl, C₃₋₆ cycloalkyl or —C₁₋₆ alkyl-C₃₋₆ cycloalkyl;
R^{8b}, R^{8d}, and R^{8e} are each independently H or C₁₋₆ alkyl;
ring B is aryl or heteroaryl having 5 to 12 ring members and 1 to 6 heteroatoms, each independently N, O or S;
the subscript m8 is an integer from 0 to 5;
each R^{8f} is independently C₁₋₆ alkyl, C₂₋₆ alkenyl, C₂₋₆ alkynyl, C₁₋₆ alkoxy, C₁₋₆ deuteroalkoxy, halo, C₁₋₆ haloalkyl, cyano, —X^{8f}-cyano, C₃₋₁₂ cycloalkyl,
—X^{8f}—C₃₋₆ cycloalkyl, —CH═CR^{8f1}R^{8f2}, heterocycloalkyl,
—X^{8f}-heterocycloalkyl, aryl, —X^{8f}-aryl, heteroaryl, or —X^{8f}-heteroaryl, wherein each heterocycloalkyl has 3 to 12 ring members and 1 to 4 heteroatoms, each independently N, O, or S, and each heteroaryl has 5 to 10 ring members and 1 to 4 heteroatoms, each independently N, O, or S, wherein each alkynyl, cycloalkyl, heterocycloalkyl, phenyl, and heteroaryl is substituted with 0 to 3 R^{8f3};
alternatively, two R^{8f} groups on adjacent ring vertices combine to form a C₃₋₆ cycloalkyl or a heterocycloalkyl having 3 to 6 ring members and 1 to 3 heteroatoms, each independently N, O or S;
each X^{8f} is independently C₁₋₆ alkylene, C₂₋₆ alkenylene, O, or S;
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3};
each R^{8f3} is independently C₁₋₆ alkyl, —Y⁸—C₁₋₆ alkyl, C₁₋₆ deuteroalkyl,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, C₁₋₆ alkoxy, halo, C₁₋₆ haloalkyl, —Y⁸—C₁₋₆ haloalkyl, cyano, —C₁₋₆ alkyl-cyano, oxo, C₃₋₆ cycloalkyl, —X^{8f3}—C₃₋₆ cycloalkyl, heterocycloalkyl, —X^{8f3}-heterocycloalkyl, phenyl, —X^{8f3}-phenyl, heteroaryl, or —X^{8f3}-heteroaryl, wherein each heterocycloalkyl has 3 to 12 members and 1 to 4 heteroatoms, each independently N, O, S, or S(O)₂, and each heteroaryl has 5 to 10 ring members and 1 to 4 heteroatoms, each independently N, O, or S, and wherein nitrogen atom ring members in each heterocycloalkyl and heteroaryl are substituted with 0 to 1 C₁₋₄ alkyl;
each X^{8f3} is independently C₁₋₆ alkylene, O, C(O), or S(O)₂;
each Y⁸ is independently C(O), C(O)O, N(R^{8f4})C(O), O, S, or S(O)₂;
alternatively, two R^{8f3} groups on adjacent ring vertices combine to form a C₃₋₆ cycloalkyl or a heterocycloalkyl having 3 to 6 ring members and 1 to 3 heteroatoms, each independently N, O or S; and
each R^{8f4} is independently H or C₁₋₆ alkyl;
or a pharmaceutically acceptable salt thereof.

In some embodiments, the compound, or the pharmaceutically acceptable salt thereof, is the compound of Formula (I) having the structure of Formula (Ib)

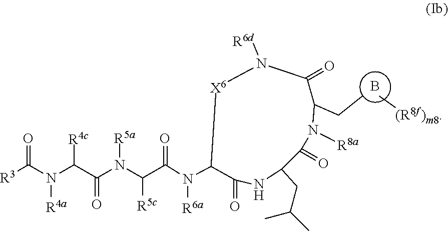

R³, R^{4a}, R^{4c}, R^{5a}, R^{5c}, R^{6a}, X⁶, R^{6d}, R^{8a}, m8, R^{8f}, and ring B can each independently be as defined for any embodiment of Formula (Ib) as described herein.

In some embodiments, the compound, or the pharmaceutically acceptable salt thereof, is the compound of Formula (I) having the structure of Formula (Ib1)

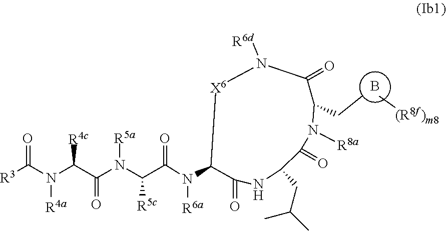

R³, R^{4a}, R^{4c}, R^{5a}, R^{5c}, R^{6a}, X⁶, R^{6d}, R^{8a}, m8, R^{8f}, and ring B can each independently be as defined for any embodiment of Formula (Ib1) as described herein.

In some embodiments, the compound, or the pharmaceutically acceptable salt thereof, is the compound of Formula (I) having the structure of Formula (Ic):

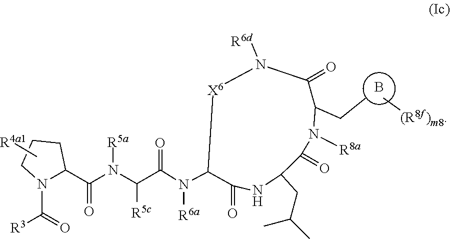

R³, R^{4a1}, R^{5a}, R^{5c}, R^{6a}, X⁶, R^{6d}, R^{8a}, m8, R^{8f}, and ring B can each independently be as defined for any embodiment of Formula (Ic) as described herein.

In some embodiments, the compound, or the pharmaceutically acceptable salt thereof, is the compound of Formula (I) having the structure of Formula (Ic1)

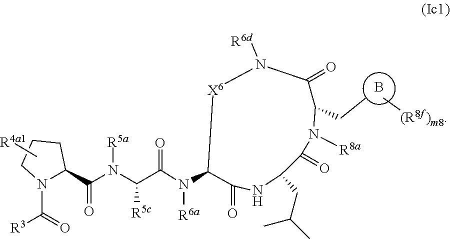

R³, R^{4a1}, R^{5a}, R^{5c}, R^{6a}, X⁶, R^{6d}, R^{8a}, m8, R^{8f}, and ring B can each independently be as defined for any embodiment of Formula (Ic1) as described herein.

In some embodiments, the compound, or the pharmaceutically acceptable salt thereof, is the compound of Formula (I) having the structure of Formula (Id)

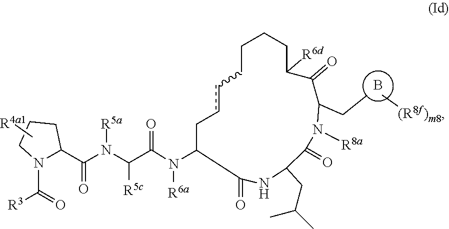

wherein the dashed bond is absent or a single bond.
R³, R^{4a1}, R^{5a}, R^{5c}, R^{6a}, R^{6d}, R^{8a}, m8, R^{8f}, and ring B can each independently be as defined for any embodiment of Formula (Id) as described herein. When the dashed line is a single bond in Formula (Id), the wavy bond attached to the double bond indicates E, Z, or a mixture of both isomers.

In some embodiments, the compound, or the pharmaceutically acceptable salt thereof, is the compound of Formula (I) having the structure of Formula (Id1)

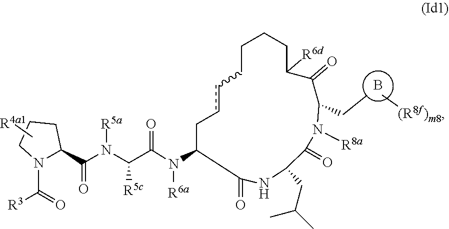

wherein the dashed bond is absent or a single bond.
R³, R^{4a1}, R^{5a}, R^{5c}, R^{6a}, R^{6d}, R^{8a}, m8, R^{8f}, and ring B can each independently be as defined for any embodiment of Formula (Id1) as described herein. When the dashed line is a bond in Formula (Id1), the wavy line attached to the double bond indicates E, Z, or a mixture or both linkages.

### Residue 3

In some embodiments, the compound, or the pharmaceutically acceptable salt thereof, is the compound of Formula (I), (Ib), (Ib1), (Ic), (Ic1), (Id), or (Id1), wherein R³ is (a) C₁₋₆ alkyl, C₂₋₆ alkenyl, C₂₋₆ alkynyl, or C₁₋₆ haloalkyl substituted with 0 to 5 R^{3a}. In some embodiments, the compound, or the pharmaceutically acceptable salt thereof, is the compound of Formula (I), (Ib), (Ib1), (Ic), (Ic1), (Id), or (Id1), wherein R³ is (a) C₁₋₆ alkyl or C₁₋₆ haloalkyl substituted with 0 to 2 R^{3a}. In some embodiments, the compound, or the pharmaceutically acceptable salt thereof, is the compound of Formula (I), (Ib), (Ib1), (Ic), (Ic1), (Id), or (Id1), wherein R³ is (a) C₁₋₆ alkyl substituted with 0 to 2 R^{3a}. In some embodiments, the compound, or the pharmaceutically acceptable salt thereof, is the compound of Formula (I), (Ib), (Ib1), (Ic), (Ic1), (Id), or (Id1), wherein R³ is (a) C₁₋₆ haloalkyl substituted with 0 to 2 R^{3a}. These embodiments of R³ can be combined with any of the embodiments described herein for R^{3a}.

In some embodiments, the compound, or the pharmaceutically acceptable salt thereof, is the compound of Formula (I), (Ib), (Ib1), (Ic), (Ic1), (Id), or (Id1), wherein each R^{3a} is —OH or C₁₋₆ alkoxy. In some embodiments, the compound, or the pharmaceutically acceptable salt thereof, is the compound of Formula (I), (Ib), (Ib1), (Ic), (Ic1), (Id), or (Id1), wherein each R^{3a} is —OH. These embodiments of R^{3a} can be combined with any of the relevant embodiments described herein for R³.

In some embodiments, the compound, or the pharmaceutically acceptable salt thereof, is the compound of Formula (I), (Ib), (Ib1), (Ic), (Ic1), (Id), or (Id1), wherein R³ is (b) C₃₋₁₂ cycloalkyl substituted with 0 to 3 R^{3b}. In some embodiments, the compound, or the pharmaceutically acceptable salt thereof, is the compound of Formula (I), (Ib), (Ib1), (Ic), (Ic1), (Id), or (Id1), wherein R³ is (b) C₃₋₇ cycloalkyl substituted with 0 to 3 R^{3b}. In some embodiments, the compound, or the pharmaceutically acceptable salt thereof, is the compound of Formula (I), (Ib), (Ib1), (Ic), (Ic1), (Id), or (Id1), wherein R³ is (b) C₅₋₆ cycloalkyl substituted with 0 to 2 R^{3b}. In some embodiments, the compound, or the pharmaceutically acceptable salt thereof, is the compound of Formula (I), (Ib), (Ib1), (Ic), (Ic1), (Id), or (Id1), wherein R³ is (b) C₃₋₇ cycloalkyl substituted with 0 to 2 R^{3b}. These embodiments of R³ can be combined with any of the embodiments described herein for R^{3b}.

In some embodiments, the compound, or the pharmaceutically acceptable salt thereof, is the compound of Formula (I), (Ib), (Ib1), (Ic), (Ic1), (Id), or (Id1), wherein each R^{3b} is halo, C₁₋₄ haloalkyl, or cyano. In some embodiments, the compound, or the pharmaceutically acceptable salt thereof, is the compound of Formula (I), (Ib), (Ib1), (Ic), (Ic1), (Id), or (Id1), wherein each R^{3b} is halo or C₁₋₄ haloalkyl. In some embodiments, the compound, or the pharmaceutically acceptable salt thereof, is the compound of Formula (I), (Ib), (Ib1), (Ic), (Ic1), (Id), or (Id1), wherein each R^{3b} is fluoro or trifluoromethyl. These embodiments of R^{3b} can be combined with any of the relevant embodiments described herein for R³.

In some embodiments, the compound, or the pharmaceutically acceptable salt thereof, is the compound of Formula (I), (Ib), (Ib1), (Ic), (Ic1), (Id), or (Id1), wherein R³ is (c) heterocycloalkyl having 3 to 6 ring members and 1 to 3 heteroatoms, each independently N, O, or S, wherein the heterocycloalkyl is substituted with 0 to 5 R^{3c}. In some embodiments, the compound, or the pharmaceutically acceptable salt thereof, is the compound of Formula (I), (Ib), (Ib1), (Ic), (Ic1), (Id), or (Id1), wherein R³ is (c) heterocycloalkyl having 6 ring members and 1 to 3 heteroatoms, each independently N, O, or S, wherein the heterocycloalkyl is substituted with 0 to 2 R^{3c}. These embodiments of R³ can be combined with any of the relevant embodiments described herein for R^{3c}.

In some embodiments, the compound, or the pharmaceutically acceptable salt thereof, is the compound of Formula (I), (Ib), (Ib1), (Ic), (Ic1), (Id), or (Id1), wherein R^{3c} is halo or C₁₋₄ haloalkyl. In some embodiments, the compound, or the pharmaceutically acceptable salt thereof, is the compound of Formula (I), (Ib), (Ib1), (Ic), (Ic1), (Id), or (Id1), wherein each R^{3c} is fluoro or trifluoromethyl. These embodiments of R^{3c} can be combined with any of the embodiments described herein for R³.

In some embodiments, the compound, or the pharmaceutically acceptable salt thereof, is the compound of Formula (I), (Ib), (Ib1), (Ic), (Ic1), (Id), or (Id1), wherein R³ is (g) heteroaryl having 5 to 6 ring members and 1 to 3 heteroatoms, each independently N, O, or S, wherein the heteroaryl is substituted with 0, 1, 2, 3, 4, or 5 R^{3g}.

In some embodiments, the compound, or the pharmaceutically acceptable salt thereof, is the compound of Formula (I), (Ib), (Ib1), (Ic), (Ic1), (Id), or (Id1), wherein R^{3g} is independently C₁₋₆ alkyl, halo, C₁₋₆ haloalkyl, or C₃₋₆ cycloalkyl.

In some embodiments, the compound, or the pharmaceutically acceptable salt thereof, is the compound of Formula (I), (Ib), (Ib1), (Ic), (Ic1), (Id), or (Id1), wherein R³ is

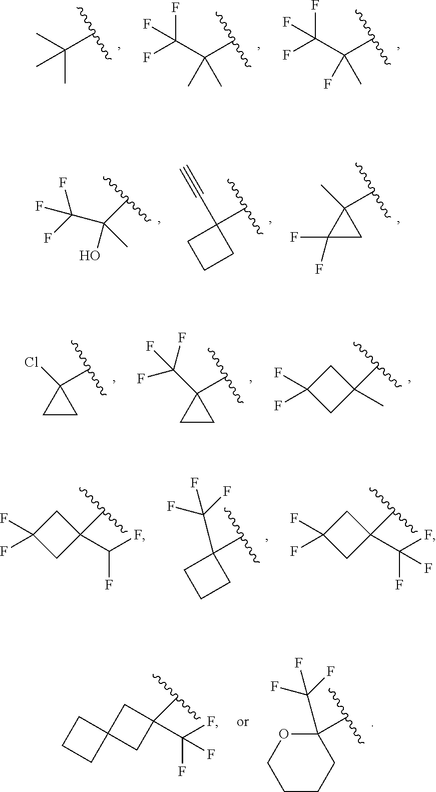

In some embodiments, the compound, or the pharmaceutically acceptable salt thereof, is the compound of Formula (I), (Ib), (Ib1), (Ic), (Ic1), (Id), or (Id1), wherein R³ is

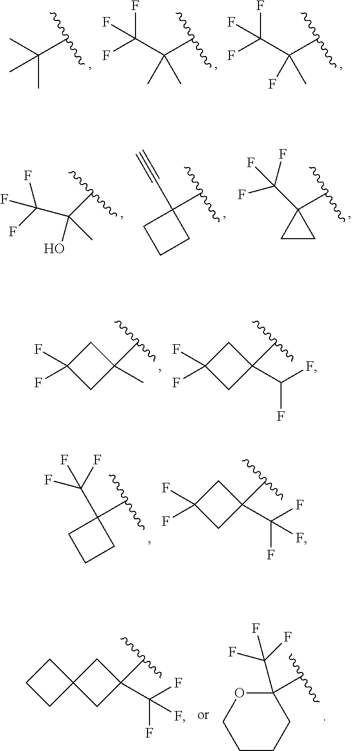

In some embodiments, the compound, or the pharmaceutically acceptable salt thereof, is the compound of Formula (I), (Ib), (Ib1), (Ic), (Ic1), (Id), or (Id1), wherein R³ is

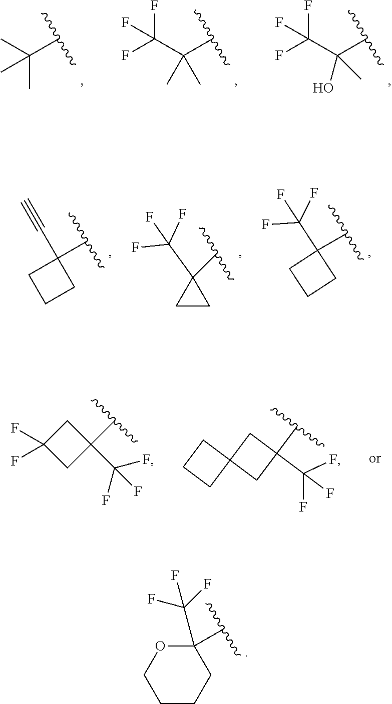

Any of the embodiments described herein for residue 3 can be combined with any of the embodiments described herein for residues 4, 5, 6, 7, and 8. For example, any of the embodiments of R³ as described herein, can be combined with any of the embodiments described herein for R^{4a}, R^{4b}, R^{4c}, R^{5a}, R^{5b}, R^{5e}, X⁶, R^{6a}, R^{6b}, R^{6d}, R^{7a}, R^{7b}, R^{7e}, R^{8a}, R^{8b}, R^{8d}, R^{8e}, ring B, m8, and R^{8f}.

### Residue 4

In some embodiments, the compound, or the pharmaceutically acceptable salt thereof, is the compound of Formula (I), (Ib), or (Ib1), wherein

R^{4a}, R^{4b}, and R^{4c} are each independently H or C₁₋₆ alkyl;
alternatively, R^{4c} and R^{4a} together with the carbon and nitrogen to which each is attached combine to form a heterocycloalkyl having 4 to 6 ring members and 1 to 3 heteroatoms, each independently N, O, or S, wherein the heterocycloalkyl is substituted with 0 to 2 R^{4a1}; and
   each R^{4a1} is independently halo.

In some embodiments, the compound, or the pharmaceutically acceptable salt thereof, is the compound of Formula (I), (Ib), or (Ib1), wherein

R^{4a}, R^{4b}, and R^{4c} are each independently H or C₁₋₆ alkyl;
alternatively, R^{4c} and R^{4a} together with the carbon and nitrogen to which each is attached combine to form a heterocycloalkyl having 4 to 6 ring members, wherein the heterocycloalkyl is substituted with 0 to 2 R^{4a1}; and
   each R^{4a1} is independently halo.

In some embodiments, the compound, or the pharmaceutically acceptable salt thereof, is the compound of Formula (I), (Ib), or (Ib1), wherein

R^{4a}, R^{4b}, and R^{4c} are each independently H or C₁₋₆ alkyl.

In some embodiments, the compound, or the pharmaceutically acceptable salt thereof, is the compound of Formula (I), (Ib), or (Ib1), wherein

R^{4b} is H or C₁₋₆ alkyl; and
R^{4c} and R^{4a} together with the carbon and nitrogen to which each is attached combine to form a heterocycloalkyl having 4 to 6 ring members and 1 to 3 heteroatoms, each independently N, O, or S, wherein the heterocycloalkyl is substituted with 0 to 3 R^{4a1}.

In some embodiments, the compound, or the pharmaceutically acceptable salt thereof, is the compound of Formula (I), (Ib), or (Ib1), wherein

R^{4b} is H or C₁₋₆ alkyl; and
R^{4c} and R^{4a} together with the carbon and nitrogen to which each is attached combine to form a heterocycloalkyl having 4 to 6 ring members, wherein the heterocycloalkyl is substituted with 0 to 3 R^{4a1}.

In some embodiments, the compound, or the pharmaceutically acceptable salt thereof, is the compound of Formula (I), (Ib), or (Ib1), wherein

R^{4a} and R^{4b} are each H; and
R^{4c} is ethyl;
alternatively, R^{4c} and R^{4a} together with the carbon and nitrogen to which each is attached combine to form a pyrrolidinyl, substituted with 0 to 2 fluoro.

In some embodiments, the compound, or the pharmaceutically acceptable salt thereof, is the compound of Formula (I), (Ib), or (ml), wherein

### R^{4b} is H; and

R^{4c} and R^{4a} together with the carbon and nitrogen to which each is attached combine to form a pyrrolidinyl, substituted with 0 to 2 R^{4a1}.

In some embodiments, the compound, or the pharmaceutically acceptable salt thereof, is the compound of Formula (I), (Ib), (Ib1), (Ic), (Ic1), (Id), (Id1), wherein R^{4a1} is C₁₋₆ alkyl or halo. In some embodiments, the compound, or the pharmaceutically acceptable salt thereof, is the compound of Formula (I), (Ib), (Ib1), (Ic), (Ic1), (Id), (Id1), wherein R^{4a1} is halo. In some embodiments, the compound, or the pharmaceutically acceptable salt thereof, is the compound of Formula (I), (Ib), (Ib1), (Ic), (Ic1), (Id), (Id1), wherein R^{4a1} is fluoro. These embodiments of R^{4a1} can be combined with any of the relevant embodiments described herein for R⁴.

The embodiments described herein for R^{4a}, R^{4b} and R^{4c} can be present in any combination. In addition, the embodiments described herein for residue 4 can be present in combination with any of the embodiments described herein for residues 3, 5, 6, 7, and 8. For example, any of the embodiments of R^{4a}, R^{4b} and R^{4c} as described herein, can be combined with any of the embodiments described herein for R³, R^{5a}, R⁵, R^{5e}, X⁶, R^{6a}, R^{6b}, R^{6d}, R^{7a}, R^{7b}, R^{7c}, R^{8a}, R^{8b}, R^{8d}, R^{8e}, ring B, m8, and R^{8f}.

### Residue 5

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, C₃₋₆ cycloalkyl, or C₁₋₄ alkyl-C₃₋₆ cycloalkyl, wherein each cycloalkyl is substituted with 0 to 3 R^{5b5}; and
   each R^{5b5} is independently C₁₋₄ alkyl, halo, or C₁₋₄ haloalkyl.

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl;
R^{5b} and R^{5c} are each independently H, —C₁₋₆ alkyl-C(O)NR^{5b1}R^{5b2}, or —C₁₋₆ alkyl-C(O)OR^{5b1};
   each R^{5b1} and R^{5b2} are independently H, C₁₋₄ alkyl, or C₁₋₄ haloalkyl;
alternatively, R^{5b1} and R^{5b2} together with the nitrogen to which each is attached combine to form a heterocycloalkyl having 3 to 6 ring members and 0 to 1 additional heteroatoms, wherein the heterocycloalkyl is substituted with 0 to 2 R^{5b3}; and
each R^{5b3} is independently C₁₋₆ alkyl, halo, C₁₋₆ haloalkyl, or cyano each R^{5b5} is independently C₁₋₄ alkyl, halo, or C₁₋₄ haloalkyl.

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl; and
R^{5b} and R^{5c} are each independently H, C₁₋₆ alkyl, C₃₋₆ cycloalkyl, C₁₋₄ alkyl-C₃₋₆ cycloalkyl.

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl;

### R^{5b} is H; and

R^{5c} is C₃₋₆ cycloalkyl or C₁₋₄ alkyl-C₃₋₆ cycloalkyl.

In some embodiments, the compound, or the pharmaceutically acceptable salt thereof, is the compound of Formula (I), (Ib), (Ib1), (Ic), (Ic1), (Id), or (Id1), wherein R^{5a} and R^{5b} are each H; and

R^{5c} is H, methyl, ethyl,

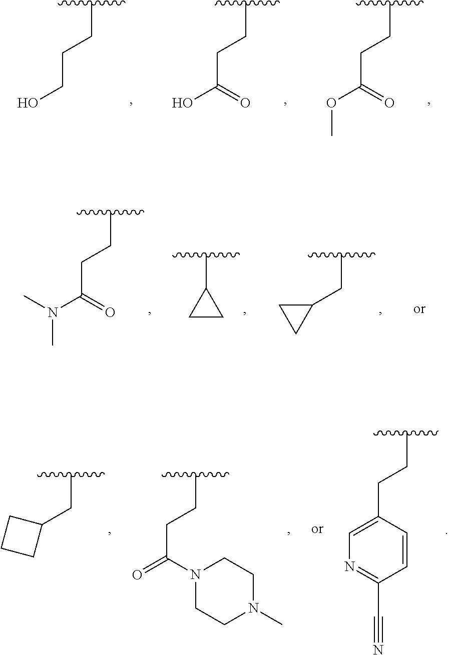

In some embodiments, the compound, or the pharmaceutically acceptable salt thereof, is the compound of Formula (I), (Ib), (Ib1), (Ic), (Ic1), (Id), or (Id1), wherein

R^{5a} and R^{5b} are each H; and
R^{5c} is H, methyl, ethyl,

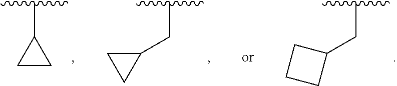

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl;

### R^{5b} is H; and

### R^{5c} is

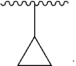 [SMILES: *C1CC1]

In some embodiments, the compound, or the pharmaceutically acceptable salt thereof, is the compound of Formula (I), (Ib), (Ib1), (Ic), (Ic1), (Id), or (Id1), wherein

R^{5a} is H or C₁₋₆ alkyl;

### R^{5b} is H; and

### R^{5c} is

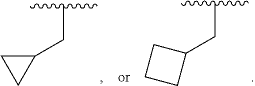 [SMILES: *CC1CC1 ; *CC1CCC1]

The embodiments described herein for R^{5a}, R^{5b} and R^{5c} can be present in any combination. In addition, the embodiments described herein for residue 5 can be present in combination with any of the embodiments described herein for residues 3, 4, 6, 7, and 8. For example, any of the embodiments of R^{5a}, R^{5b} and R^{5c} as described herein, can be combined with any of the embodiments described herein for R³, R^{4a}, R^{4b}, R^{4c}, X⁶, R^{6a}, R^{6b}, R^{6d}, R^{7a}, R^{7b}, R^{7c}, R^{8a}, R^{8b}, R^{8d}, R^{8c}, ring B, m8, and R^{8f}.

### Residue 6

In some embodiments, the compound, or the pharmaceutically acceptable salt thereof, is the compound of Formula (I), (Ib), (Ib1), (Ic), or (Ic1), wherein X⁶ is C₆₋₇ alkylene. In some embodiments, the compound, or the pharmaceutically acceptable salt thereof, is the compound of Formula (I), (Ib), (Ib1), (Ic), or (Ic1), wherein X⁶ is C₆₋₇ alkenylene. These embodiments of X⁶ can be combined with any of the embodiments described herein for R^{6a}, R^{6b} and R^{6d}.

In some embodiments, the compound, or the pharmaceutically acceptable salt thereof, is the compound of Formula (I), (Ib), (Ib1), (Ic), or (Ic1), wherein X⁶ is

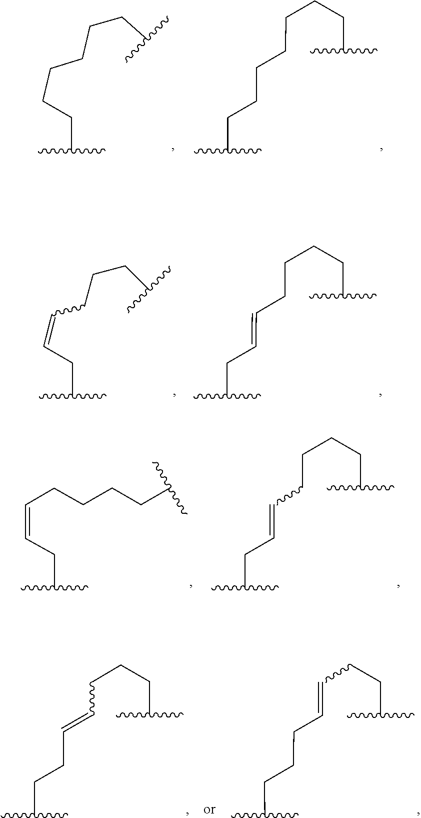

wherein the wavy bond attached to the double bond indicates E, Z, or a mixture of both isomers. These embodiments of X⁶ can be combined with any of the embodiments described herein for R^{6a}, R^{6b} and R^{6d}.

In some embodiments, the compound, or the pharmaceutically acceptable salt thereof, is the compound of Formula (I), (Ib), (Ib1), (Ic), or (Ic1), wherein X⁶ is

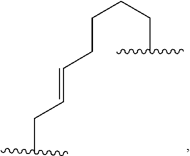 [SMILES: *C/C=C/CCCC*]

wherein the wavy bond attached to the double bond indicates E, Z, or a mixture of both isomers. This embodiment of X⁶ can be combined with any of the embodiments described herein for R^{6a}, R^{6b} and R^{6d}.

In some embodiments, the compound, or the pharmaceutically acceptable salt thereof, is the compound of Formula (I), (Ib), (Ib1), (Ic), or (Ic1), wherein X⁶ is

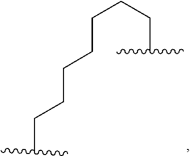 [SMILES: *CCCCCCC*]

This embodiment of X⁶ can be combined with any of the embodiments described herein for R^{6a}, R^{6b} and R^{6d}.

In some embodiments, the compound, or the pharmaceutically acceptable salt thereof, is the compound of Formula (I), (Ib), (Ib1), (Ic), (Ic1), (Id), or (Id1), wherein R^{6a} is H, C₁₋₄ alkyl, C₁₋₄ deuteroalkyl, or —C₁₋₄ alkyl-C₃₋₆ cycloalkyl; and R^{6b} and R^{6d} are each independently H or C₁₋₆ alkyl. In some embodiments, the compound, or the pharmaceutically acceptable salt thereof, is the compound of Formula (I), (Ib), (Ib1), (Ic), (Ic1), (Id), or (Id1),

wherein
R^{6a} is H, C₁₋₄ alkyl, or —C₁₋₄ alkyl-C₃₋₆ cycloalkyl; and R^{6b} and R^{6d} are each independently H or C₁₋₆ alkyl. In some embodiments, the compound, or the pharmaceutically acceptable salt thereof, is the compound of Formula (I), (Ib), (Ib1), (Ic), (Ic1), (Id), or (Id1), wherein
R^{6a} is C₁₋₄ alkyl; R^{6b} is H; and R^{6d} is C₁₋₄ alkyl. In some embodiments, the compound, or the pharmaceutically acceptable salt thereof, is the compound of Formula (I), (Ib), (Ib1), (Ic), (Ic1), (Id), or (Id1), wherein R^{6a} is C₁₋₄ alkyl; and R^{6b} and R^{6d} are each H. In some embodiments, the compound, or the pharmaceutically acceptable salt thereof, is the compound of Formula (I), (Ib), (Ib1), (Ic), (Ic1), (Id), or (Id1), wherein R^{6a}, R^{6b}, and R^{6d} are each H. These embodiments of R^{6a}, R^{6b} and R^{6d} can be combined with any of the embodiments described herein for X⁶.

In some embodiments, the compound, or the pharmaceutically acceptable salt thereof, is the compound of Formula (I), (Ib), (Ib1), (Ic), (Ic1), (Id), or (Id1), wherein R^{6a} is H, methyl, ethyl,

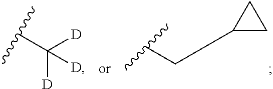

R^{6b} is H; and R^{6d} is H, methyl, or ethyl. This embodiment of R^{6a}, R^{6b} and R^{6d} can be combined with any of the embodiments described herein for X⁶.

In some embodiments, the compound, or the pharmaceutically acceptable salt thereof, is the compound of Formula (I), (Ib), (Ib1), (Ic), (Ic1), (Id), or (Id1), wherein R^{6a} is H, methyl, ethyl, or

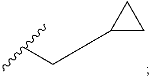 [SMILES: *CCC1CC1]

R^{6b} is H; and R^{6d} is H, methyl, or ethyl. This embodiment of R^{6a}, R^{6b} and R^{6d} can be combined with any of the embodiments described herein for X⁶.

In some embodiments, the compound, or the pharmaceutically acceptable salt thereof, is the compound of Formula (I), (Ib), (Ib1), (Ic), (Ic1), (Id), or (Id1), wherein R^{6a} is H, methyl, ethyl, or

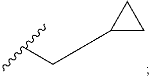 [SMILES: *CCC1CC1]

R^{6b} is H; and R^{6d} is H or methyl. This embodiment of R^{6a}, R^{6b} and R^{6d} can be combined with any of the embodiments described herein for X⁶.

The embodiments described herein for X⁶, R^{6a}, R^{6b} and R^{6d} can be present in any combination. In addition, the embodiments described herein for residue 6 can be present in combination with any of the embodiments described herein for residues 3, 4, 5, 7, and 8. For example, any of the embodiments of X⁶, R^{6a}, R^{6b} and R^{6d} as described herein, can be combined with any of the embodiments described herein for R³, R^{4a}, R^{4b}, R⁴, R^{5a}, R^{5b}, R⁵, R^{7a}, R^{7b}, R^{7e}, R^{8a}, R^{8b}, R^{8d}, R^{8e}, ring B, m8, and R^{8f}.

### Residue 7

In some embodiments, the compound, or the pharmaceutically acceptable salt thereof, is the compound of Formula (I), wherein R^{7a} and R^{7b} are each independently H or C₁₋₆ alkyl; and R^{7c} is C₁₋₆ alkyl, C₂₋₆ alkenyl, C₁₋₆ haloalkyl, C₁₋₆ alkyl-OH, or

—C₁₋₆ alkyl-C₃₋₆ cycloalkyl.

In some embodiments, the compound, or the pharmaceutically acceptable salt thereof, is the compound of Formula (I), wherein R^{7a} and R^{7b} are each independently H or C₁₋₆ alkyl; and R^{7c} is C₁₋₆ alkyl or C₁₋₆ haloalkyl.

In some embodiments, the compound, or the pharmaceutically acceptable salt thereof, is the compound of Formula (I), wherein R^{7a} and R^{7b} are each H; and R^{7c} is C₁₋₆ alkyl.

In some embodiments, the compound, or the pharmaceutically acceptable salt thereof, is the compound of Formula (I), wherein R^{7a} and R^{7b} are each H; and R^{7c} is isobutyl,

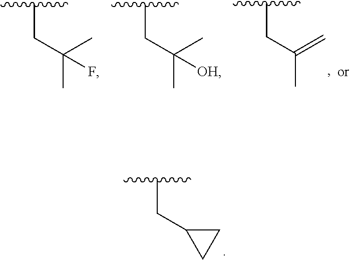

In some embodiments, the compound, or the pharmaceutically acceptable salt thereof, is the compound of Formula (I), wherein R^{7a} and R^{7b} are each H; and R^{7c} is isobutyl.

The embodiments described herein for R^{7a}, R^{7b} and R^{7c} can be present in any combination. In addition, the embodiments described herein for residue 7 can be present in combination with any of the embodiments described herein for residues 3, 4, 5, 6, and 8. For example, any of the embodiments of R^{7a}, R^{7b} and R^{7c} as described herein, can be combined with any of the embodiments described herein for R³, R^{4a}, R^{4b}, R^{4e}, R^{5a}, R^{5b}, R⁵⁰, X⁶, R^{6a}, R^{6b}, R^{6d}, R^{8a}, R^{8b}, R^{8d}, R^{8e}, ring B, m8, and R^{8f}.

### Residue 8

In some embodiments, the compound, or the pharmaceutically acceptable salt thereof, is the compound of Formula (I), (Ib), (Ib1), (Ic), (Ic1), (Id), or (Id1), wherein R^{8a}, R^{8b}, R^{8d}, and R^{8e} are each independently H or C₁₋₆ alkyl. In some embodiments, the compound, or the pharmaceutically acceptable salt thereof, is the compound of Formula (I), (Ib), (Ib1), (Ic), (Ic1), (Id), or (Id1), wherein R^{8a} is H or methyl; and R^{8b}, R^{8d} and R^{8e} are each H. These embodiments of R^{8a}, R^{8b}, R^{8d}, and R^{8e} can be combined with any of the embodiments described herein for m8, ring B, R^{8f}, and R^{8f3}.

In some embodiments, the compound, or the pharmaceutically acceptable salt thereof, is the compound of Formula (I), (Ib), (Ib1), (Ic), (Ic1), (Id), or (Id1), wherein ring B is phenyl or heteroaryl having 5 to 6 ring members and 1 to 3 heteroatoms, wherein each heteroatom is N, O, or S. In some embodiments, the compound, or the pharmaceutically acceptable salt thereof, is the compound of Formula (I), (Ib), (Ib1), (Ic), (Ic1), (Id), or (Id1), wherein ring B is phenyl. In some embodiments, the compound, or the pharmaceutically acceptable salt thereof, is the compound of Formula (I), (Ib), (Ib1), (Ic), (Ic1), (Id), or (Id1), wherein ring B is naphthyl. In some embodiments, the compound, or the pharmaceutically acceptable salt thereof, is the compound of Formula (I), (Ib), (Ib1), (Ic), (Ic1), (Id), or (Id1), wherein ring B is biphenyl. In some embodiments, the compound, or the pharmaceutically acceptable salt thereof, is the compound of Formula (I), (Ib), (Ib1), (Ic), (Ic1), (Id), or (Id1), wherein ring B is a heteroaryl having 5 to 6 ring members and 1 to 3 heteroatoms, wherein each heteroatom is N, O, or S. In some embodiments, the compound, or the pharmaceutically acceptable salt thereof, is the compound of Formula (I), (Ib), (Ib1), (Ic), (Ic1), (Id), or (Id1), wherein ring B is thiophenyl. In some embodiments, the compound, or the pharmaceutically acceptable salt thereof, is the compound of Formula (I), (Ib), (Ib1), (Ic), (Ic1), (Id), or (Id1), wherein ring B is a heteroaryl having 5 to 6 ring members and 1 to 3 heteroatoms, wherein each heteroatom is N. In some embodiments, the compound, or the pharmaceutically acceptable salt thereof, is the compound of Formula (I), (Ib), (Ib1), (Ic), (Ic1), (Id), or (Id1), wherein ring B is pyridyl. In some embodiments, the compound, or the pharmaceutically acceptable salt thereof, is the compound of Formula (I), (Ib), (Ib1), (Ic), (Ic1), (Id), or (Id1), wherein ring B is benzofuranyl. In some embodiments, the compound, or the pharmaceutically acceptable salt thereof, is the compound of Formula (I), (Ib), (Ib1), (Ic), (Ic1), (Id), or (Id1), wherein ring B is indolyl. In some embodiments, the compound, or the pharmaceutically acceptable salt thereof, is the compound of Formula (I), (Ib), (Ib1), (Ic), (Ic1), (Id), or (Id1), wherein ring B is indazolyl. In some embodiments, the compound, or the pharmaceutically acceptable salt thereof, is the compound of Formula (I), (Ib), (Ib1), (Ic), (Ic1), (Id), or (Id1), wherein ring B is quinolinyl. In some embodiments, the compound, or the pharmaceutically acceptable salt thereof, is the compound of Formula (I), (Ib), (Ib1), (Ic), (Ic1), (Id), or (Id1), wherein ring B is pyrid-3-yl. In some embodiments, the compound, or the pharmaceutically acceptable salt thereof, is the compound of Formula (I), (Ib), (Ib1), (Ic), (Ic1), (Id), or (Id1), wherein ring B is phenyl or pyridyl. In some embodiments, the compound, or the pharmaceutically acceptable salt thereof, is the compound of Formula (I), (Ib), (Ib1), (Ic), (Ic1), (Id), or (Id1), wherein ring B is phenyl or pyrid-3-yl. These embodiments of ring B can be combined with any of the embodiments described herein for R^{8a}, R^{8b}, R^{8d}, R^{8e}, m8, R^{8f}, and R^{8f3}.

In some embodiments, the compound, or the pharmaceutically acceptable salt thereof, is the compound of Formula (I), (Ib), (Ib1), (Ic), (Ic1), (Id), or (Id1), wherein the subscript m8 is 1, 2, or 3. In some embodiments, the compound, or the pharmaceutically acceptable salt thereof, is the compound of Formula (I), (Ib), (Ib1), (Ic), (Ic1), (Id), or (Id1), wherein the subscript m8 is 0. In some embodiments, the compound, or the pharmaceutically acceptable salt thereof, is the compound of Formula (I), (Ib), (Ib1), (Ic), (Ic1), (Id), or (Id1), wherein the subscript m8 is 1. In some embodiments, the compound, or the pharmaceutically acceptable salt thereof, is the compound of Formula (I), (Ib), (Ib1), (Ic), (Ic1), (Id), or (Id1), wherein the subscript m8 is 2. In some embodiments, the compound, or the pharmaceutically acceptable salt thereof, is the compound of Formula (I), (Ib), (Ib1), (Ic), (Ic1), (Id), or (Id1), wherein the subscript m8 is 3. In some embodiments, the compound, or the pharmaceutically acceptable salt thereof, is the compound of Formula (I), (Ib), (Ib1), (Ic), (Ic1), (Id), or (Id1), wherein the subscript m8 is 4. In some embodiments, the compound, or the pharmaceutically acceptable salt thereof, is the compound of Formula (I), (Ib), (Ib1), (Ic), (Ic1), (Id), or (Id1), wherein the subscript m8 is 5. These embodiments of m8 can be combined with any of the embodiments described herein for R^{8a}, R^{8b}, R^{8d}, R^{8e}, R^{8f}, R⁸⁰, and ring B.

In some embodiments, the compound, or the pharmaceutically acceptable salt thereof, is the compound of Formula (I), (Ib), (Ib1), (Ic), (Ic1), (Id), or (Id1), wherein the moiety

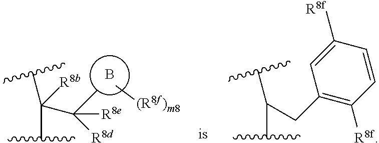

These embodiments can be combined with any of the embodiments described herein for R^{8a}, R^{8b}, R^{8d}, R^{8e}, R^{8f}, R^{8f3}, m8 and ring B.

In some embodiments, the compound, or the pharmaceutically acceptable salt thereof, is the compound of Formula (I), (Ib), (Ib1), (Ic), (Ic1), (Id), or (Id1), wherein the moiety

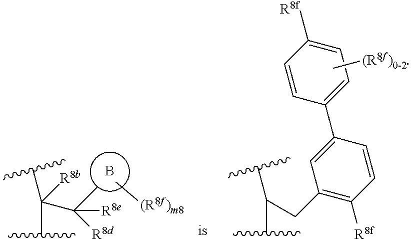

These embodiments can be combined with any of the embodiments described herein for R^{8a}, R^{8b}, R^{8d}, R^{8e}, R^{8f}, R^{8f3}, m8 and ring B.

In some embodiments, the compound, or the pharmaceutically acceptable salt thereof, is the compound of Formula (I), (Ib), (Ib1), (Ic), (Ic1), (Id), or (Id1), wherein the moiety

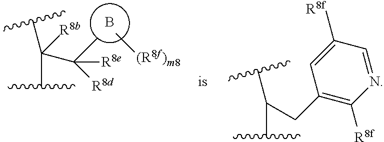

These embodiments can be combined with any of the embodiments described herein for R^{8a}, R^{8b}, R^{8d}, R⁸, R^{8f}, R^{8f3}, m8 and ring B.

In some embodiments, at least one R^{8f} is halo. In some embodiments, at least one R^{8f} is fluoro or chloro. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently C₁₋₆ alkyl, C₂₋₆ alkenyl, C₁₋₆ alkoxy, C₁₋₆ deuteroalkoxy, halo, C₁₋₆ haloalkyl, cyano, or —X^{8f}-cyano. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —X^{8f}—C₃₋₆ cycloalkyl, —CH═CR^{8f1}R^{8f2}, heterocycloalkyl, —X^{8f}-heterocycloalkyl, phenyl, —X^{8f}-phenyl, heteroaryl, or —X^{8f}-heteroaryl, wherein each heterocycloalkyl has 3 to 10 ring members and 1 to 3 heteroatoms, each independently N, O, or S, and each heteroaryl has 5 to 10 ring members and 1 to 3 heteroatoms, each independently N, O, or S, wherein each cycloalkyl, heterocycloalkyl, phenyl, and heteroaryl is substituted with 0 to 3 R^{8f3}; each X^{8f} is independently C₁₋₆ alkylene, C₂₋₆ alkenylene, O, or S; and each R^{8f1} and R^{8f} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —X^{8f}—C₃₋₆ cycloalkyl, —CH═CR^{8f1}R^{8f2}, heterocycloalkyl, —X^{8f}-heterocycloalkyl, heteroaryl, or —X^{8f}-heteroaryl, wherein each heterocycloalkyl has 3 to 10 ring members and 1 to 3 heteroatoms, each independently N, O, or S, and each heteroaryl has 5 to 10 ring members and 1 to 3 heteroatoms, each independently N, O, or S, wherein each cycloalkyl, heterocycloalkyl, and heteroaryl is substituted with 0 to 3 R^{8f3}; each X^{8f} is independently C₁₋₆ alkylene, C₂₋₆ alkenylene, O, or S; and each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each X^{8f} is independently C₁₋₆ alkylene. In some embodiments, the compound, or the pharmaceutically acceptable salt thereof, is the compound of Formula (I), (Ib), (Ib1), (Ic), (Ic1), (Id), or (Id1), wherein each X^{8f} is independently C₂₋₆ alkenylene. In some embodiments, the compound, or the pharmaceutically acceptable salt thereof, is the compound of Formula (I), (Ib), (Ib1), (Ic), (Ic1), (Id), or (Id1), wherein each X^{8f} is independently —O—C₁₋₆ alkylene. In some embodiments, the compound, or the pharmaceutically acceptable salt thereof, is the compound of Formula (I), (Ib), (Ib1), (Ic), (Ic1), (Id), or (Id1), wherein each X^{8f} is independently C(O). In some embodiments, the compound, or the pharmaceutically acceptable salt thereof, is the compound of Formula (I), (Ib), (Ib1), (Ic), (Ic1), (Id), or (Id1), wherein each X^{8f} is independently O. In some embodiments, the compound, or the pharmaceutically acceptable salt thereof, is the compound of Formula (I), (Ib), (Ib1), (Ic), (Ic1), (Id), or (Id1), wherein each X^{8f} is independently S. In some embodiments, the compound, or the pharmaceutically acceptable salt thereof, is the compound of Formula (I), (Ib), (Ib1), (Ic), (Ic1), (Id), or (Id1), wherein each X^{8f} is independently C₁₋₆ alkylene or O. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —CH═CR^{8f1}R^{8f2}, heterocycloalkyl, phenyl, or heteroaryl, wherein each heterocycloalkyl has 3 to 10 ring members and 1 to 3 heteroatoms, each independently N, O, or S, and each heteroaryl has 5 to 10 ring members and 1 to 3 heteroatoms, each independently N, O, or S, wherein each cycloalkyl, heterocycloalkyl, phenyl, and heteroaryl is substituted with 0 to 3 R^{8f3}; each R^{8f1} and R^{8f} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently C₁₋₄ alkyl, C₂₋₄ alkenyl, C₁₋₄ alkoxy, C₁₋₄ deuteroalkoxy, halo, C₁₋₄ haloalkyl, cyano, or —C₁₋₂ alkyl-cyano. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —O—C₃₋₆ cycloalkyl, heterocycloalkyl, —C₂₋₄ alkenyl-heterocycloalkyl, —O-heterocycloalkyl, —CH═CR^{8f1}R^{8f2}, phenyl, —O-phenyl, heteroaryl, or —O-heteroaryl, wherein each heterocycloalkyl has 3 to 9 ring members and 1 to 3 heteroatoms, each independently N, O, or S, and each heteroaryl has 5 to 9 ring members and 1 to 3 heteroatoms, each independently N, O, or S, wherein each cycloalkyl, heterocycloalkyl, phenyl, and heteroaryl is substituted with 0 to 3 R^{8f3}; and R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —O—C₃₋₆ cycloalkyl, heterocycloalkyl, —C₂₋₄ alkenyl-heterocycloalkyl, —O-heterocycloalkyl, phenyl, —O-phenyl, heteroaryl, or —O— heteroaryl, wherein each heterocycloalkyl has 3 to 6 ring members and 1 to 2 heteroatoms, each independently N, O, or S, and each heteroaryl has 5 to 6 ring members and 1 to 3 heteroatoms, each independently N, O, or S, wherein each cycloalkyl, heterocycloalkyl, phenyl, and heteroaryl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, C₃₋₆ cycloalkyl, —O—C₃₋₆ cycloalkyl, heteroaryl, or —O-heteroaryl, wherein each heteroaryl has 5 to 6 ring members and 1 to 3 heteroatoms, each independently N, O, or S, wherein each cycloalkyl, and heteroaryl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, heterocycloalkyl, —O-heterocycloalkyl, phenyl, or —O-phenyl, wherein each heterocycloalkyl has 3 to 6 ring members and 1 to 2 heteroatoms, each independently N, O, or S, wherein each heterocycloalkyl and phenyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f} is independently halo, heterocycloalkyl, or —O-heterocycloalkyl, wherein each heterocycloalkyl has 3 to 6 ring members and 1 to 2 heteroatoms, each independently N, O, or S, wherein each heterocycloalkyl is substituted with 0 to 3 R^{8f3}. These embodiments of R^{8f} can be combined with any of the embodiments described herein for R^{8a}, R^{8b}, R^{8d}, R^{8e}, m8, ring B, and R^{8f3}.

In some embodiments, the compound, or the pharmaceutically acceptable salt thereof, is the compound of Formula (I), (Ib), (Ib1), (Ic), (Ic1), (Id), or (Id1), wherein each

R^{8f3} is independently C₁₋₆ alkyl, —Y⁸—C₁₋₆ alkyl, C₁₋₆ deuteroalky,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, halo, C₁₋₆ haloalkyl, —Y⁸—C₁₋₆ haloalkyl, or oxo; each Y⁸ is independently C(O), C(O)O, N(R^{8f4})C(O), O, S, or S(O)₂; and each R^{8f4} is independently H or C₁₋₆ alkyl. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each

R^{8f3} is independently C₁₋₆ alkyl, —Y⁸—C₁₋₆ alkyl, C₁₋₆ deuteroalky,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, halo, C₁₋₆ haloalkyl, —Y⁸—C₁₋₆ haloalkyl, —C₁₋₆ alkyl-NR^{8g}R^{8h}, or oxo; each Y⁸ is independently C(O), C(O)O, N(R^{8f4})C(O), O, S, or S(O)₂; and each R^{8f4} is independently H or C₁₋₆ alkyl. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each each R^{8g} and R^{8h} is independently H, C₁₋₃ alkyl, or C₁₋₃ haloalkyl.

In some embodiments, the compound, or the pharmaceutically acceptable salt thereof, is the compound of Formula (I), (Ib), (Ib1), (Ic), (Ic1), (Id), or (Id1), wherein each Y⁸ is independently —Y⁸—C₁₋₆ alkyl. In some embodiments, the compound, or the pharmaceutically acceptable salt thereof, is the compound of Formula (I), (Ib), (Ib1), (Ic), (Ic1), (Id), or (Id1), wherein each Y⁸ is independently —Y⁸-methyl. In some embodiments, the compound, or the pharmaceutically acceptable salt thereof, is the compound of Formula (I), (Ib), (Ib1), (Ic), (Ic1), (Id), or (Id1), wherein each Y⁸ is independently C(O). In some embodiments, the compound, or the pharmaceutically acceptable salt thereof, is the compound of Formula (I), (Ib), (Ib1), (Ic), (Ic1), (Id), or (Id1), wherein each Y⁸ is independently C(O)O. In some embodiments, the compound, or the pharmaceutically acceptable salt thereof, is the compound of Formula (I), (Ib), (Ib1), (Ic), (Ic1), (Id), or (Id1), wherein each Y⁸ is independently NHC(O). In some embodiments, the compound, or the pharmaceutically acceptable salt thereof, is the compound of Formula (I), (Ib), (Ib1), (Ic), (Ic1), (Id), or (Id1), wherein each Y⁸ is independently O. In some embodiments, the compound, or the pharmaceutically acceptable salt thereof, is the compound of Formula (I), (Ib), (Ib1), (Ic), (Ic1), (Id), or (Id1), wherein each Y⁸ is independently S. In some embodiments, the compound, or the pharmaceutically acceptable salt thereof, is the compound of Formula (I), (Ib), (Ib1), (Ic), (Ic1), (Id), or (Id1), wherein each Y⁸ is independently S(O)₂. These embodiments of Y⁸ can be combined with any of the embodiments described herein for R^{8a}, R^{8b}, R^{8d}, R^{8e}, m8, ring B, R^{8f}, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₁₋₆ alkyl, C₁₋₆ deuteroalky, —OH, —C₁₋₆ alkyl-OH, halo, C₁₋₆ haloalkyl, or oxo. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₁₋₆ alkyl or —Y⁸—C₁₋₆ alkyl. In some embodiments, the compound, or the pharmaceutically acceptable salt thereof, is the compound of Formula (I), (Ib), (Ib1), (Ic), (Ic1), (Id), or (Id1), wherein each Y⁸ is independently a C(O) or C(O)O. These embodiments of R^{8f3} and Y⁸ can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₃₋₆ cycloalkyl, —X^{8f3}—C₃₋₆ cycloalkyl, heterocycloalkyl, or —X^{8f3}-heterocycloalkyl, wherein each heterocycloalkyl has 3 to 6 members and 1 to 2 heteroatoms, each independently N, O, S, or S(O)₂; and each X^{8f3} is independently C₁₋₆ alkylene, C(O), or S(O)₂. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each X^{8f3} is independently C₁₋₆ alkylene. In some embodiments, the compound, or the pharmaceutically acceptable salt thereof, is the compound of Formula (I), (Ib), (Ib1), (Ic), (Ic1), (Id), or (Id1), wherein each X^{8f3} is independently C(O). In some embodiments, the compound, or the pharmaceutically acceptable salt thereof, is the compound of Formula (I), (Ib), (Ib1), (Ic), (Ic1), (Id), or (Id1), wherein each X^{8f3} is independently S(O)₂. These embodiments of X^{8f3} can be combined with any of the embodiments described herein for R^{8a}, R^{8b}, R^{8d}, R^{8e}, m8, ring B, R^{8f}, and R^{8f3}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₃₋₆ cycloalkyl or heterocycloalkyl, wherein each heterocycloalkyl has 3 to 6 members and 1 to 2 heteroatoms, each independently N, O, S, or S(O)₂. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein two R^{8f3} groups on adjacent ring vertices combine to form a heterocycloalkyl having 3 to 6 ring members and 1 to 3 heteroatoms, each independently N, O or S. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₁₋₄ alkyl, C₁₋₄ alkoxy, C₂₋₆ alkoxyalkyl, —S(O)₂—C₁₋₄ alkyl, —C₁₋₄ alkyl-S(O)₂—C₁₋₄ alkyl, halo, C₁₋₄ haloalkyl, oxo, —C(O)—C₁₋₄ alkyl, or —C(O)O—C₁₋₄ alkyl. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein each R^{8f3} is independently C₁₋₄ alkyl, C₁₋₄ alkoxy, C₂₋₆ alkoxyalkyl, halo, C₁₋₄ haloalkyl, oxo, —S(O)₂—C₁₋₄ alkyl, —C₁₋₄ alkyl-S(O)₂—C₁₋₄ alkyl, —C(O)—C₁₋₄ alkyl, —C(O)O—C₁₋₄ alkyl, C₃₋₆ cycloalkyl, —C(O)—C₃₋₆ cycloalkyl, —S(O)₂—C₃₋₆ cycloalkyl, heterocycloalkyl, —C₁₋₄ alkyl-heterocycloalkyl, or —S(O)₂-heterocycloalkyl, wherein each heterocycloalkyl has 4 to 6 members and 1 to 2 heteroatoms, each independently N, O, S, or S(O)₂; alternatively, two R^{8f3} groups on adjacent ring vertices combine to form a non-aromatic cyclic moiety having 3 to 6 ring members and 0 to 2 additional heteroatoms, each independently N, O or S. These embodiments of R^{8f3} can be combined with any of the embodiments described herein for R^{8a}, R^{8b}, R^{8d}, R^{8e}, m8, ring B, and R^{8f}.

In some embodiments, the compound, or the pharmaceutically acceptable salt thereof, is the compound of Formula (I), (Ib), (Ib1), (Ic), (Ic1), (Id), or (Id1), wherein
m8 is 1, 2, or 3; and
each R^{8f} is independently methyl, isopropyl, isobutyl, methoxy, ethoxy, fluoro, chloro, cyano,

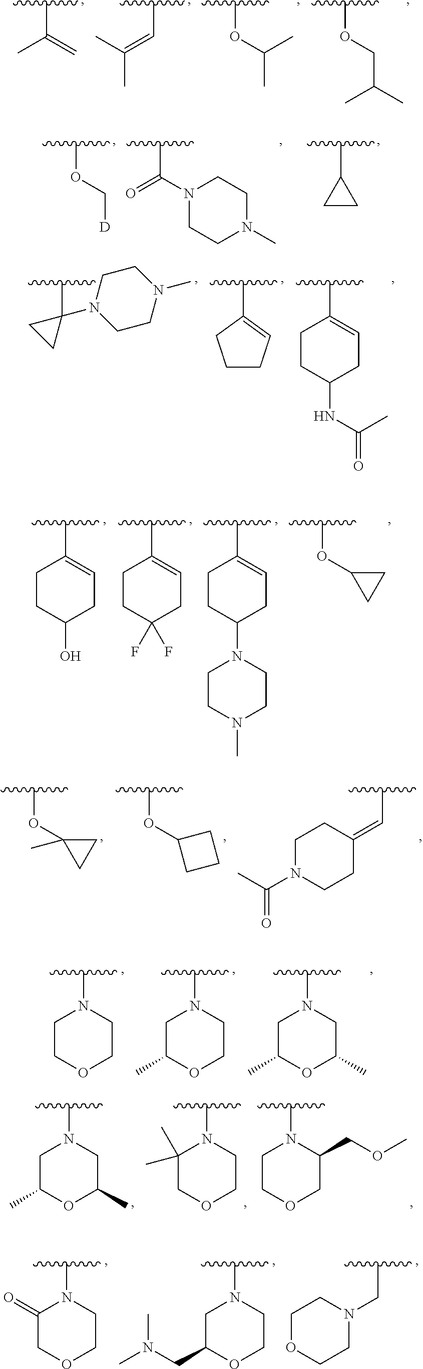

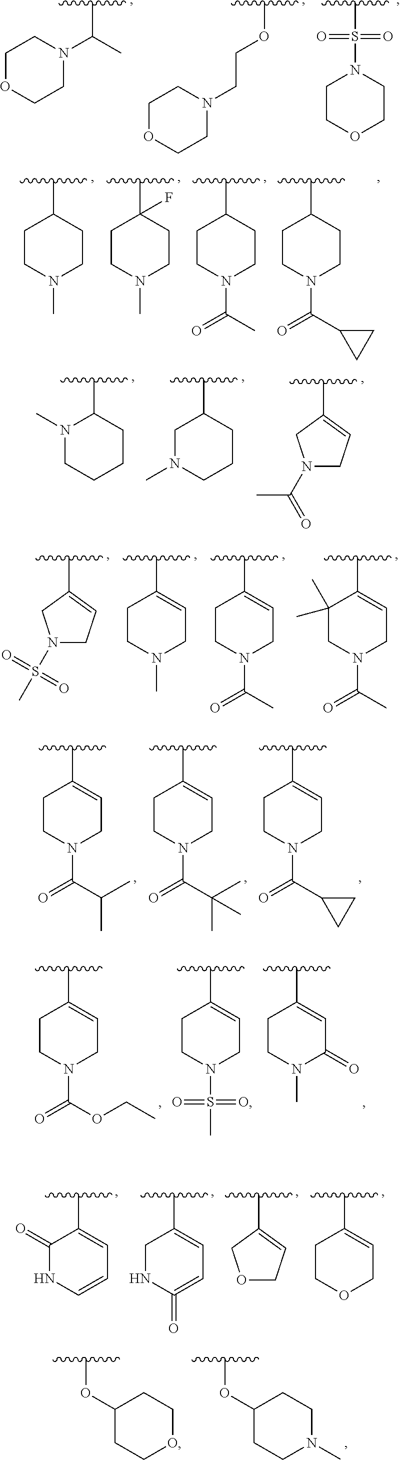

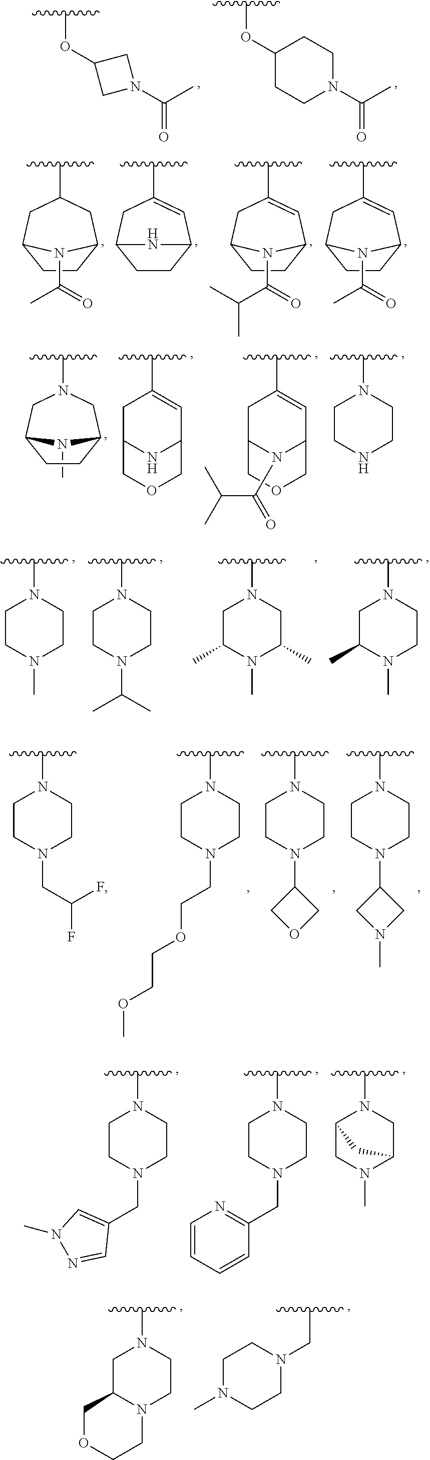

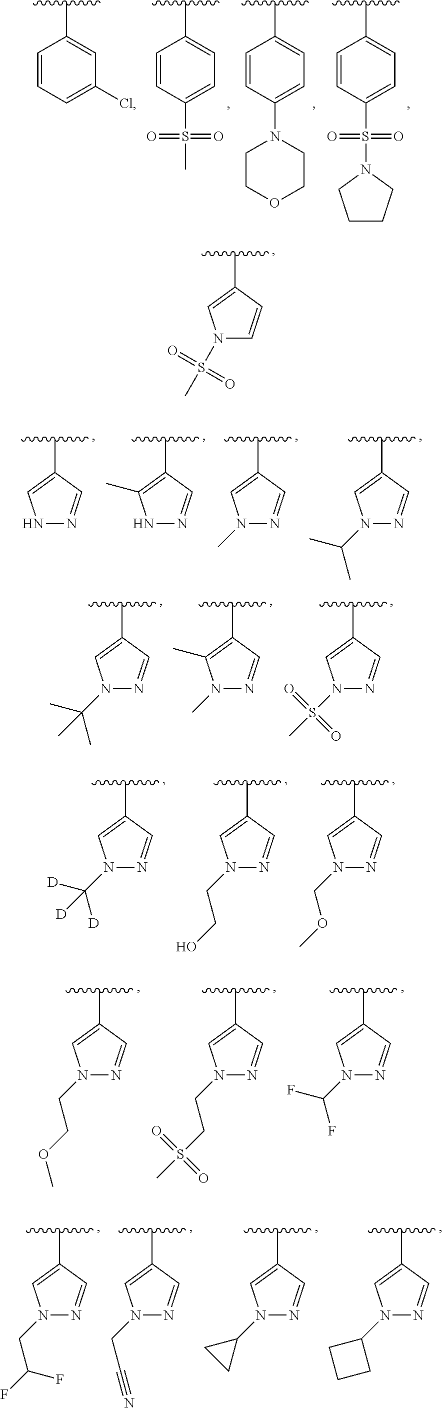

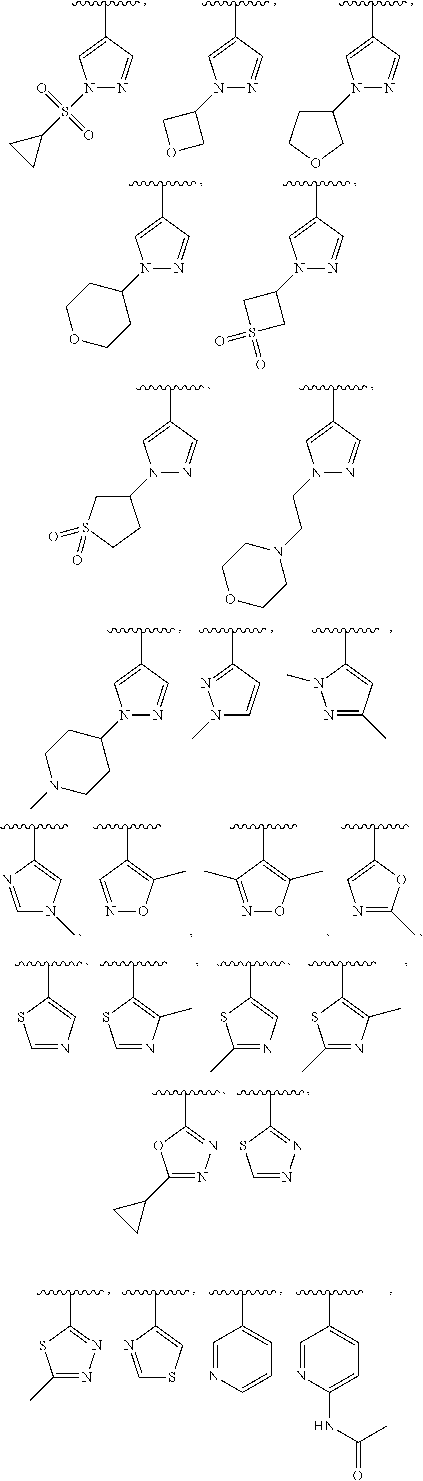

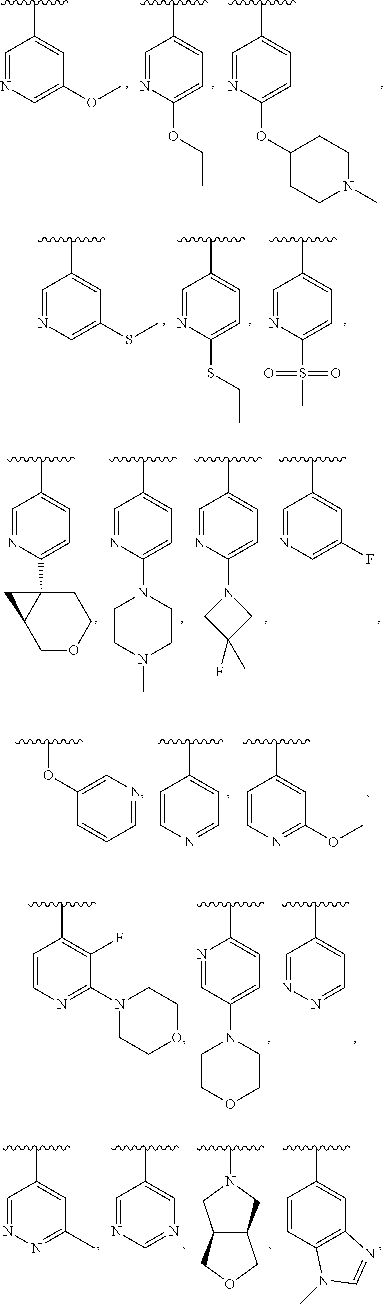

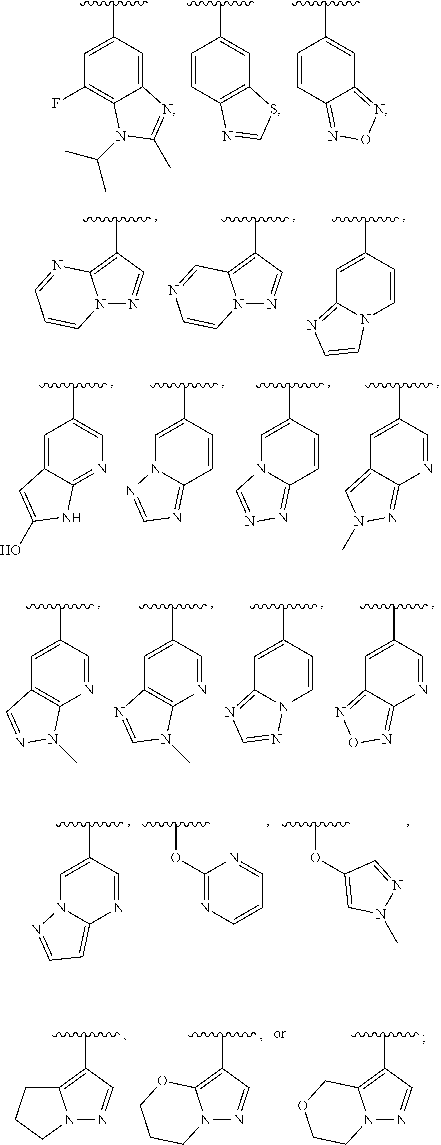

alternatively, ring B and three R^{8f} groups form the moiety

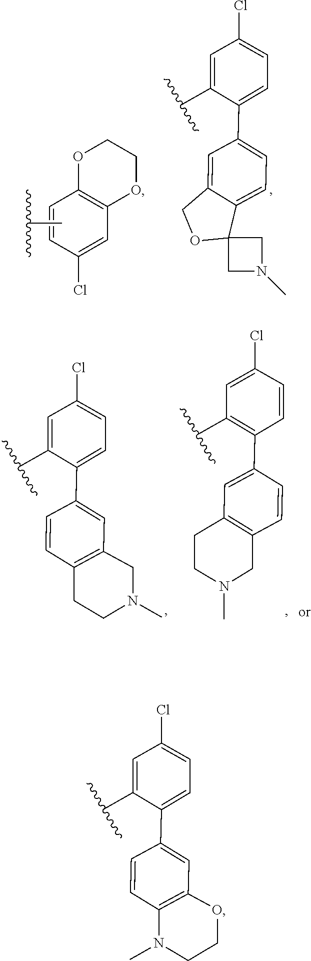

wherein the wavy line identifies attachment to the remainder of the molecule.

In some embodiments, the compound, or the pharmaceutically acceptable salt thereof, is the compound of Formula (I), (Ib), (Ib1), (Ic), (Ic1), (Id), or (Id11), wherein
m8 is 1, 2, or 3; and
each R^{8f} is independently methyl, isopropyl, isobutyl, methoxy, ethoxy, fluoro, chloro, cyano,

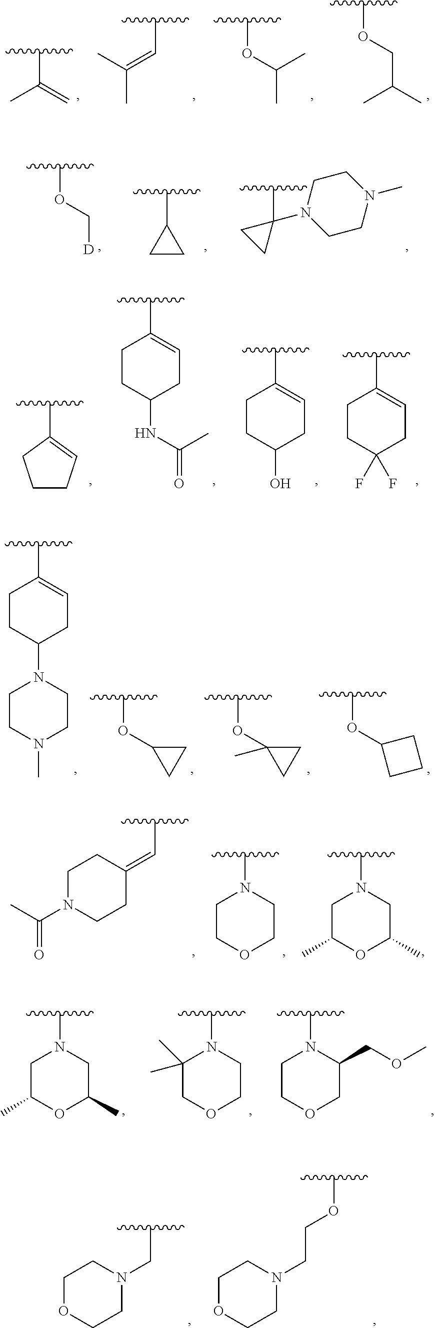

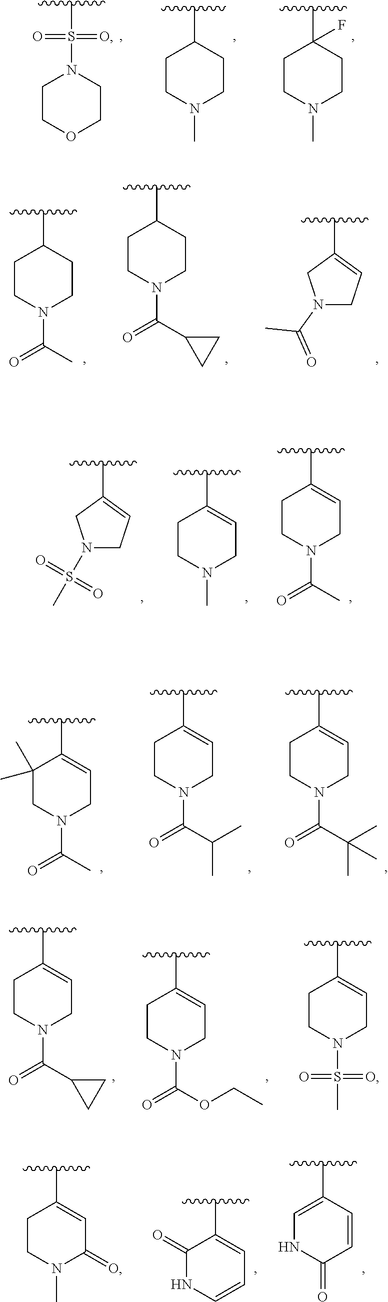

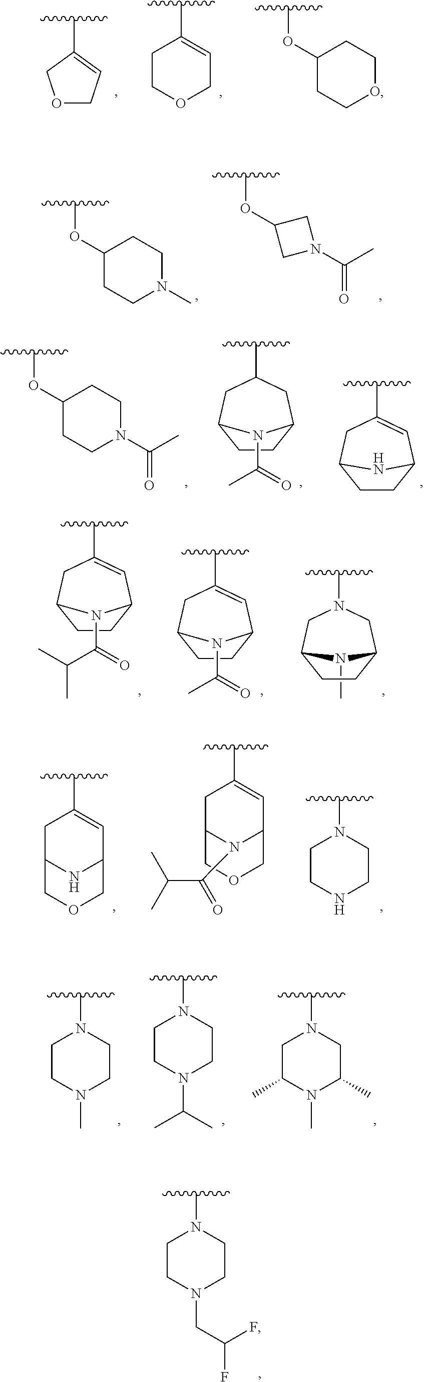

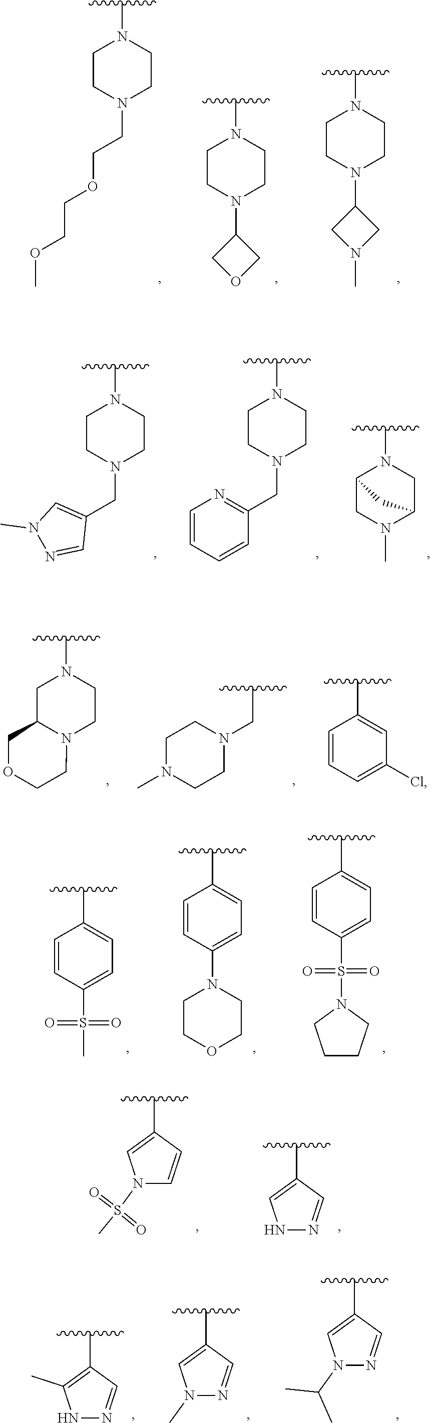

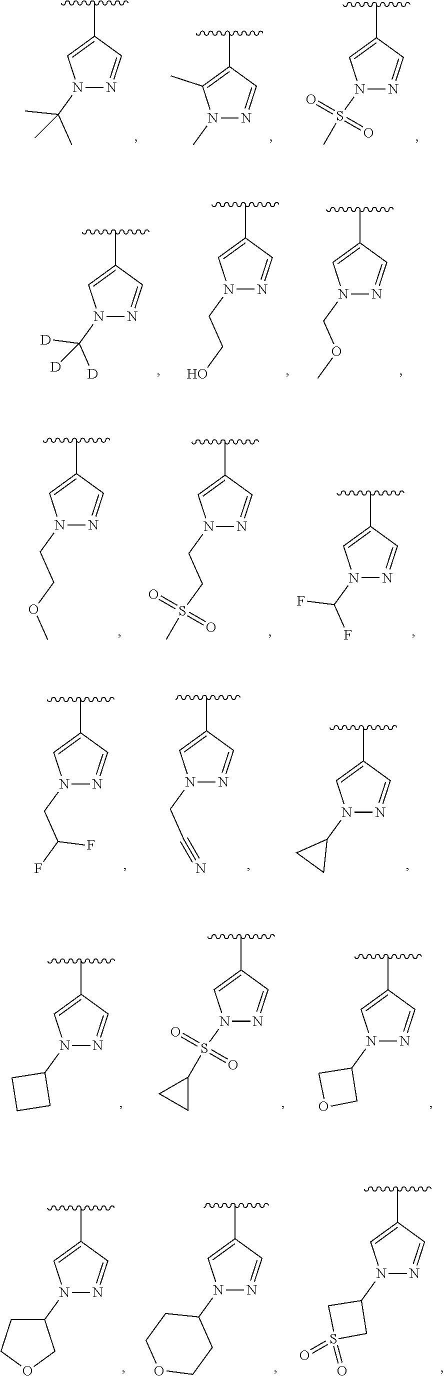

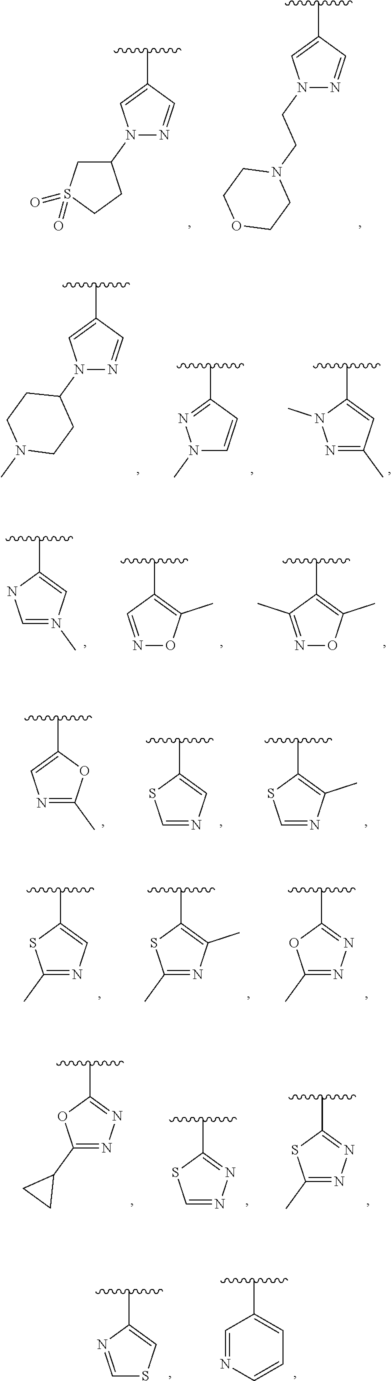

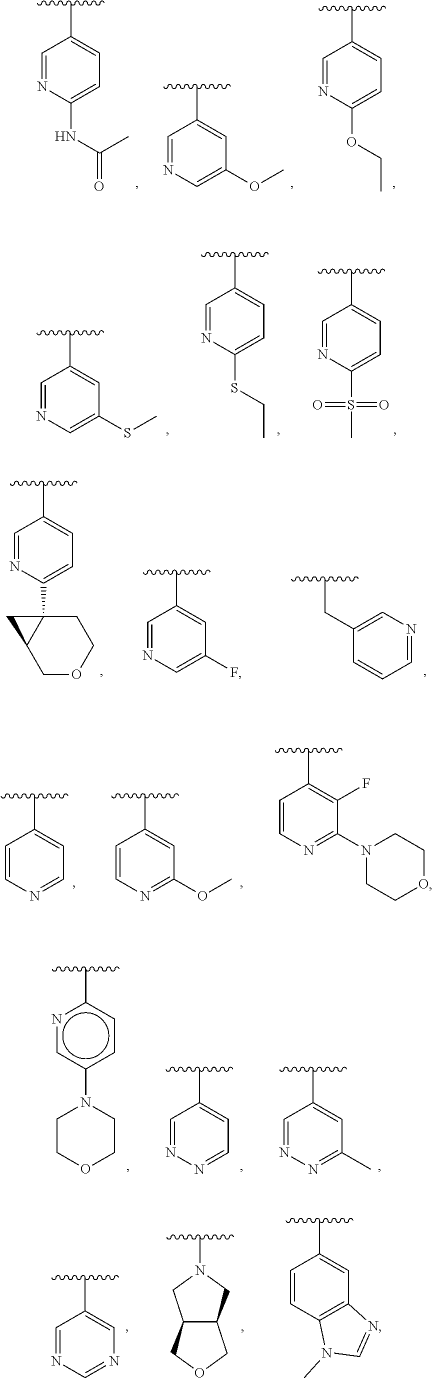

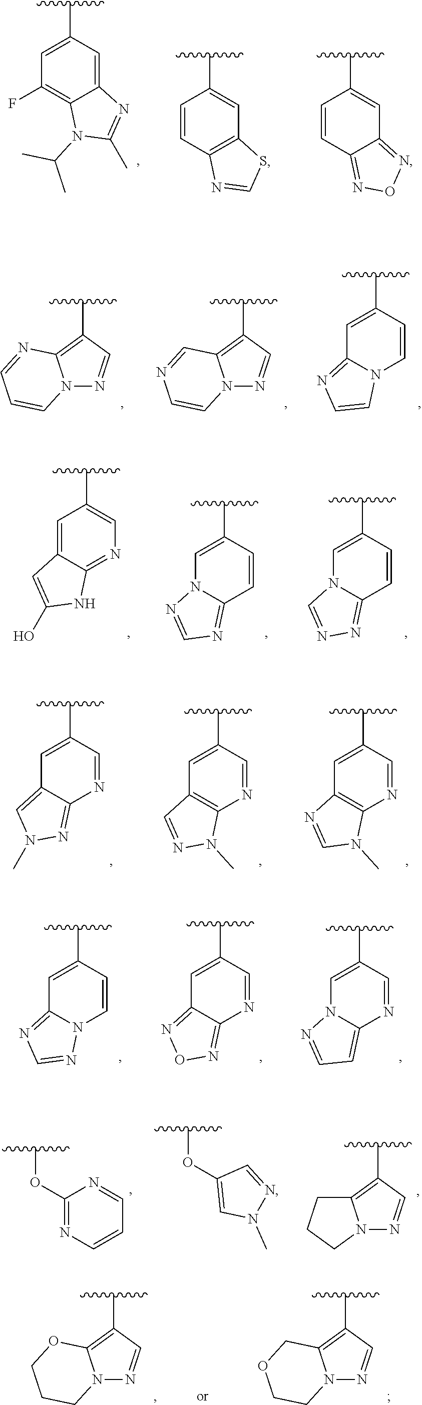

alternatively, ring B and three R^{8f} groups form the moiety

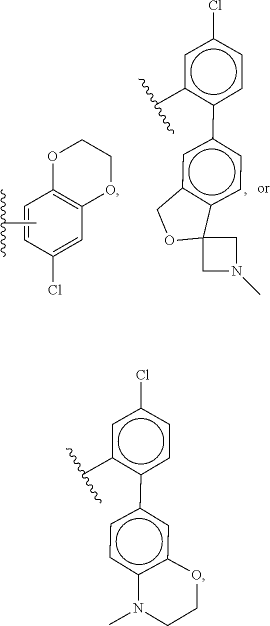

wherein the wavy line identifies attachment to the remainder of the molecule.

In some embodiments, the compound, or the pharmaceutically acceptable salt thereof, is the compound of Formula (I), (Ib), (Ib1), (Ic), (Ic1), (Id), or (Id1), wherein
m8 is 1, 2, or 3; and
each R^{8f} is independently methyl, isopropyl, isobutyl, methoxy, ethoxy, fluoro, chloro,

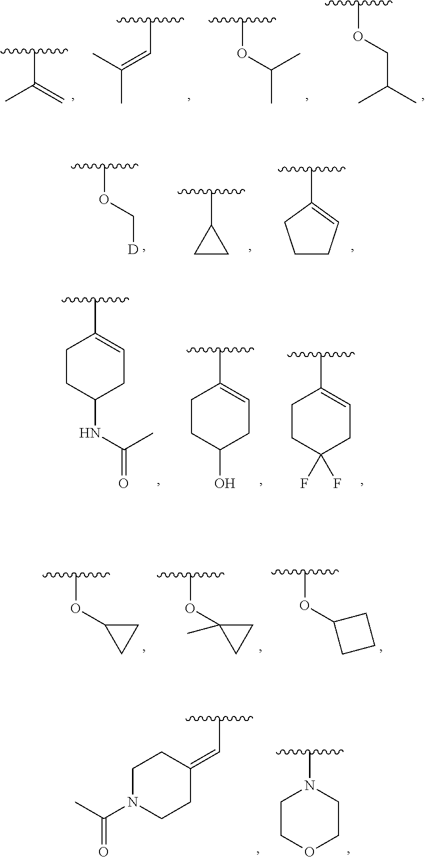

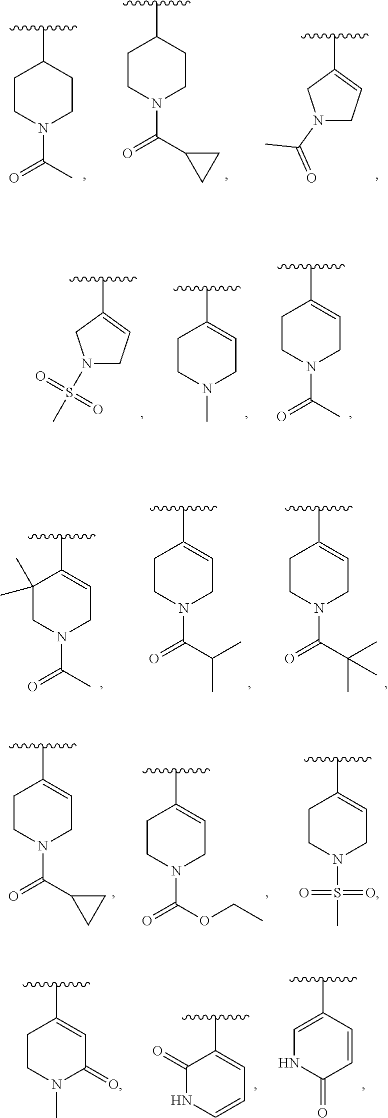

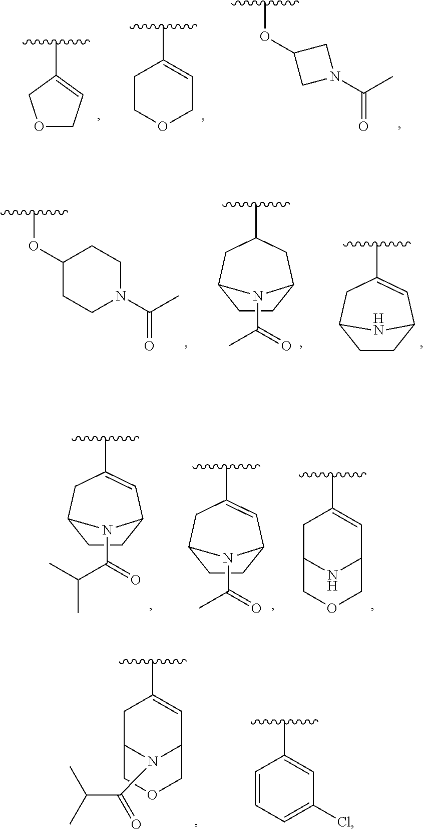

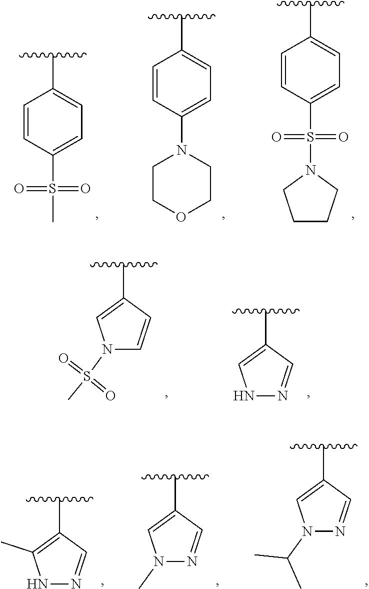

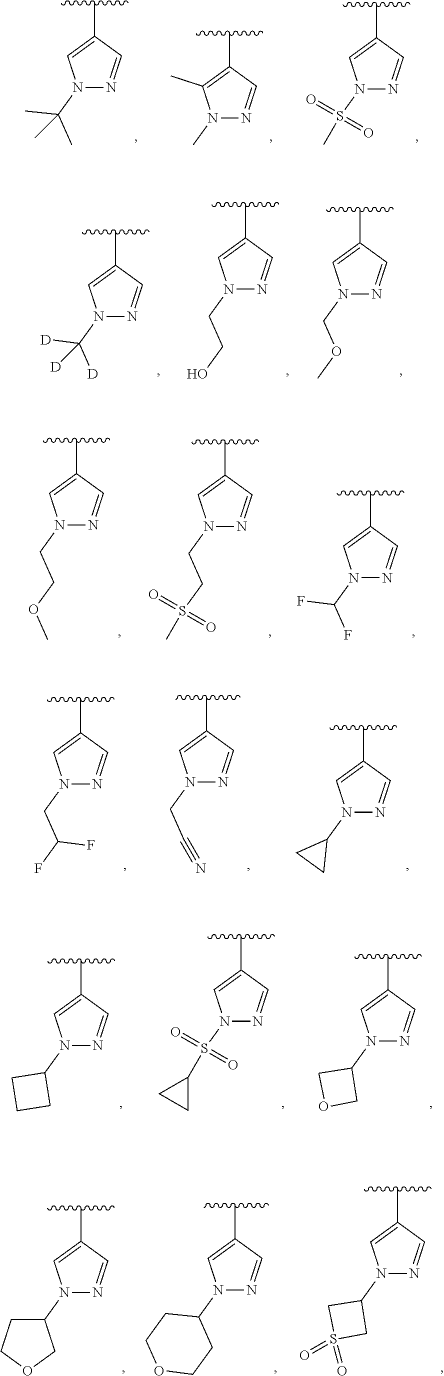

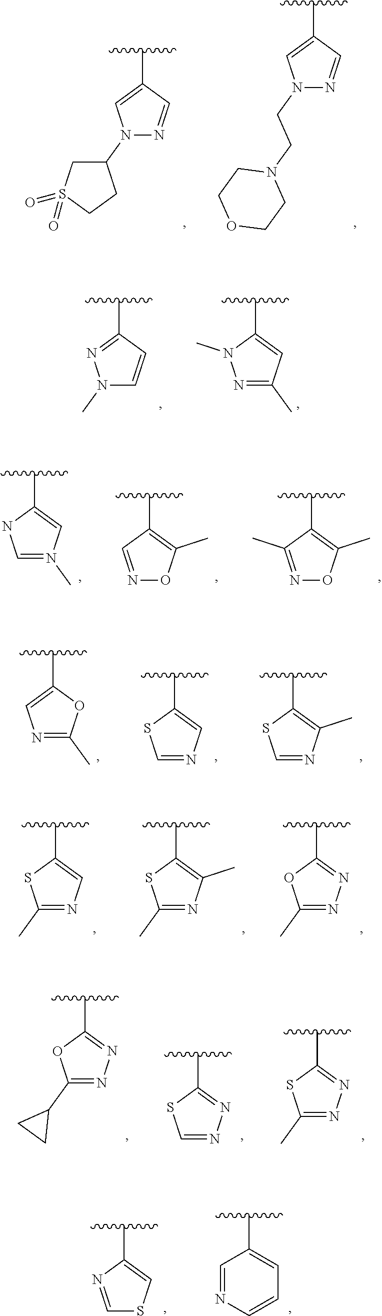

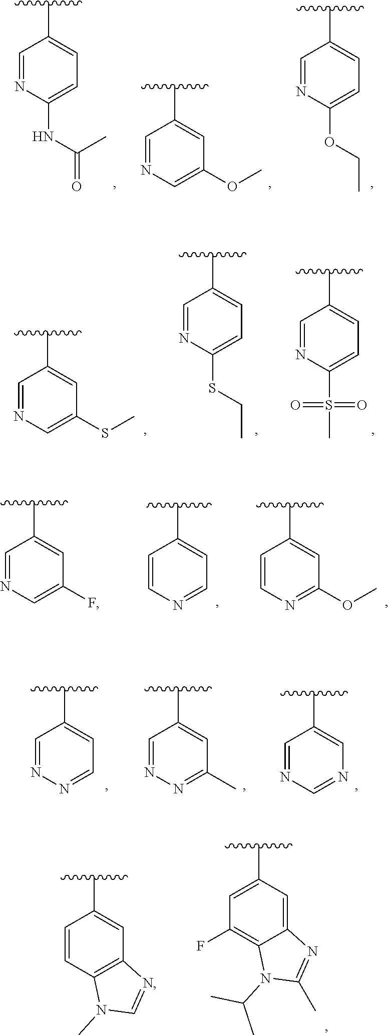

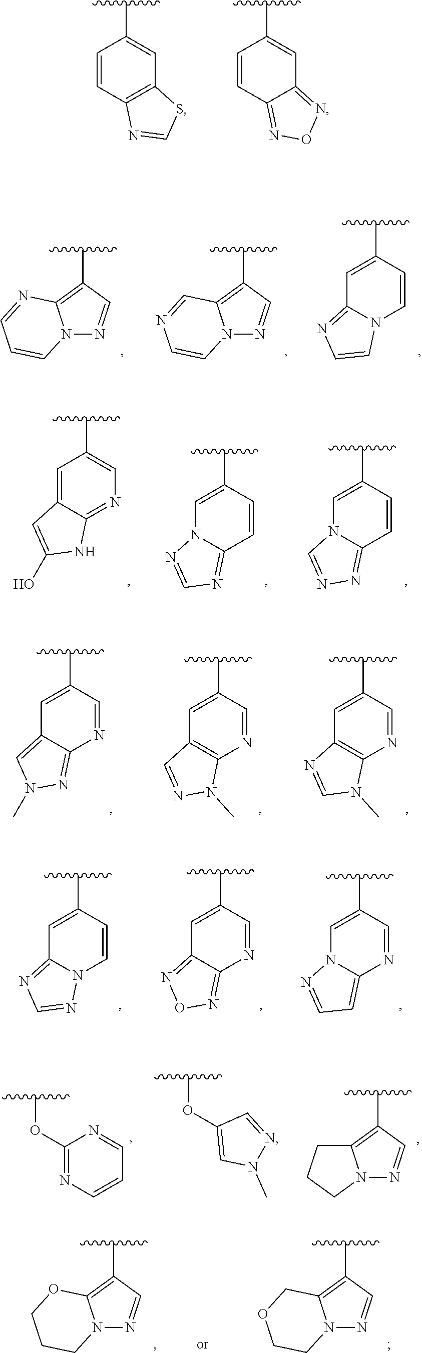

alternatively, ring B and three R^{8f} groups form the moiety

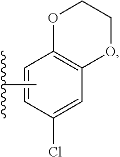

wherein the wavy line identifies attachment to the remainder of the molecule.

The embodiments described herein for R^{8a}, R^{8b}, R^{8d}, R^{8e}, m8, R^{8f}, and ring B can be present in any combination. In addition, the embodiments described herein for residue 8 can be present in combination with any of the embodiments described herein for residues 3, 4, 5, 6, and 7. For example, any of the embodiments of R^{8a}, R^{8b}, R^{8d}, R^{8e}, m8, R^{8f}, and ring B as described herein, can be combined with any of the embodiments described herein for R³, R^{4a}, R^{4b}, R^{5a}, R^{5b}, R^{5c}, X⁶, R^{6a}, R^{6b}, R^{6d}, R^{7a}, R^{7b}, and R^{7c}.

### Residues 3 to 8

In some embodiments, the compound, or the pharmaceutically acceptable salt thereof, is the compound of Formula (I), (Ib), (Ib1), (Ic), (Ic1), (Id), or (Id1), wherein
R³ is

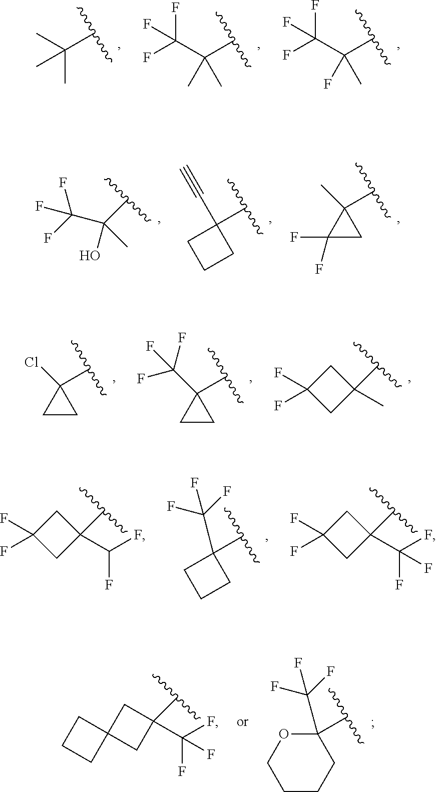

R^{4a} and R^{4b} are each H;
R^{4c} is ethyl;
alternatively, R^{4c} and R^{4a} together with the carbon and nitrogen to which each is attached combine to form a pyrrolidinyl, substituted with 0 to 2 fluoro;
R^{5a} and R^{5b} are each H;
R^{5c} is H, methyl, ethyl,

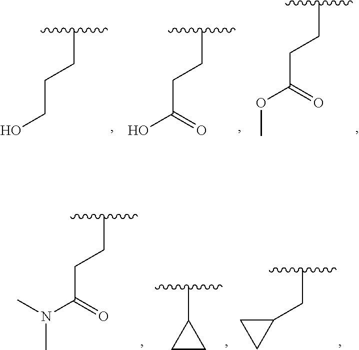

X⁶ is

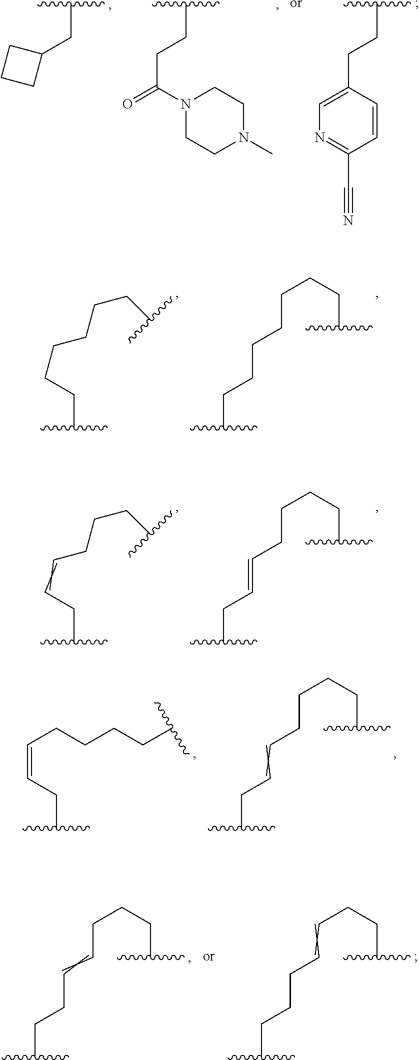

R^{6a} is H, methyl, ethyl,

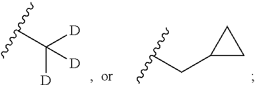 [SMILES: *C([2H])([2H])[2H] ; *CC1CC1]

R^{6b} is H;
R^{6d} is H, methyl, or ethyl;
R^{7a} and R^{7b} are each H;
R^{7c} is isobutyl,

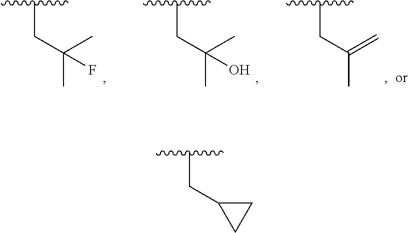

R^{8a} is H or methyl;
R^{8b}, R^{8d} and R^{8e} are each H;
ring B is phenyl, naphthyl, biphenyl, thiophenyl, pyridyl, benzofuranyl, indolyl, indazolyl, or quinolinyl;
m8 is 1, 2, or 3; and
each R^{8f} is independently methyl, isopropyl, isobutyl, methoxy, ethoxy, fluoro, chloro, cyano,

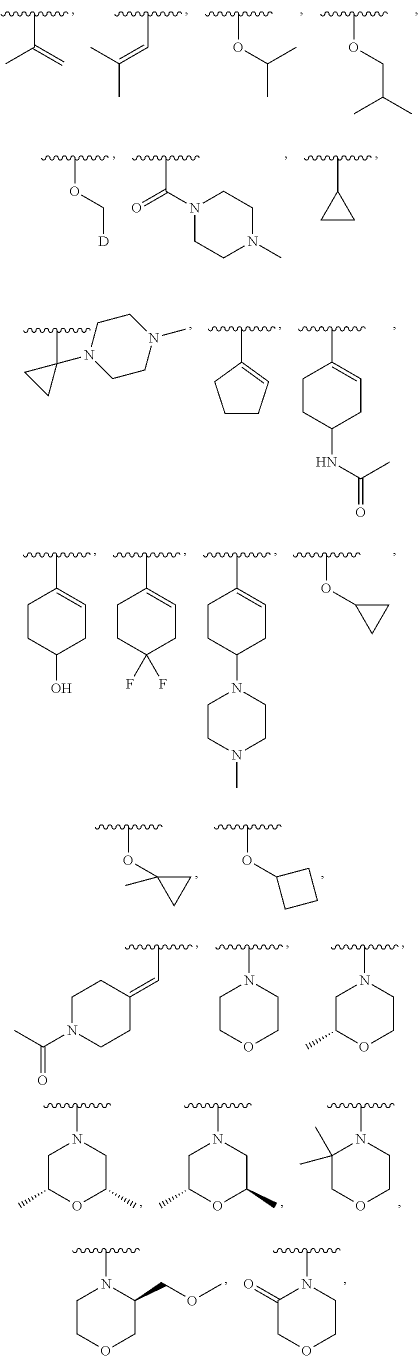

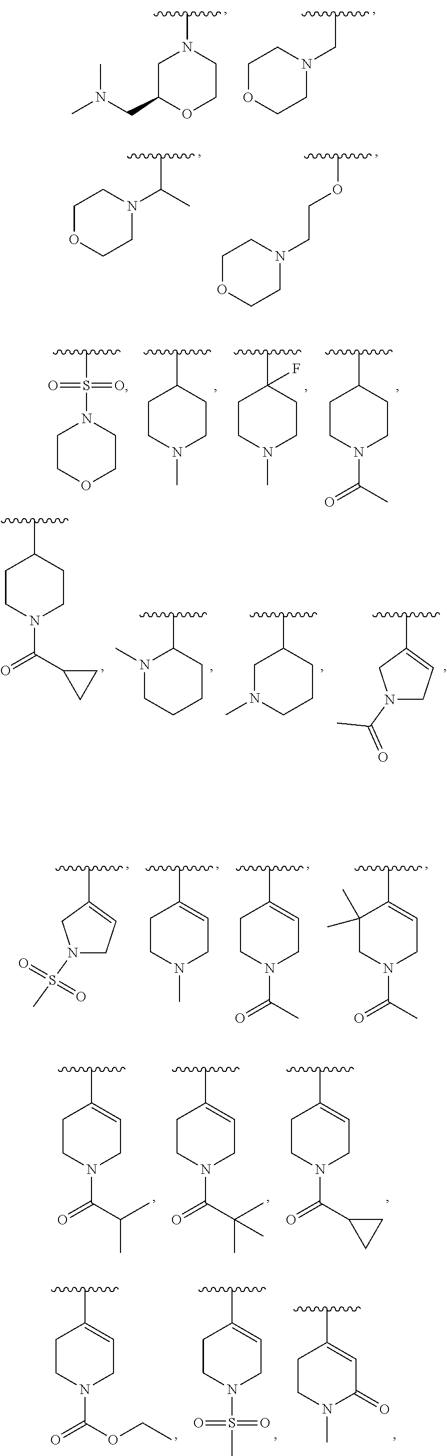

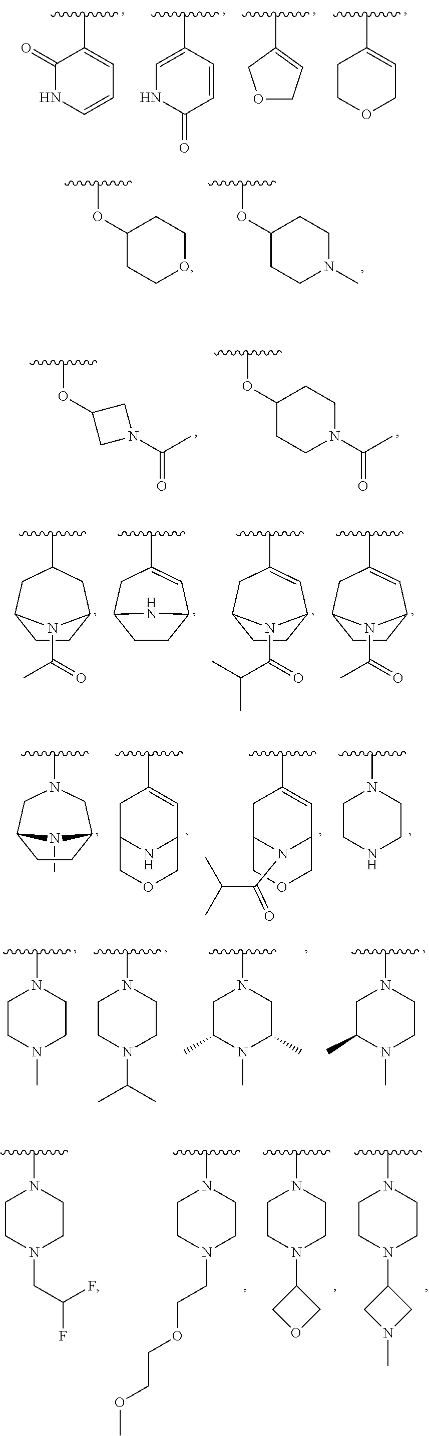

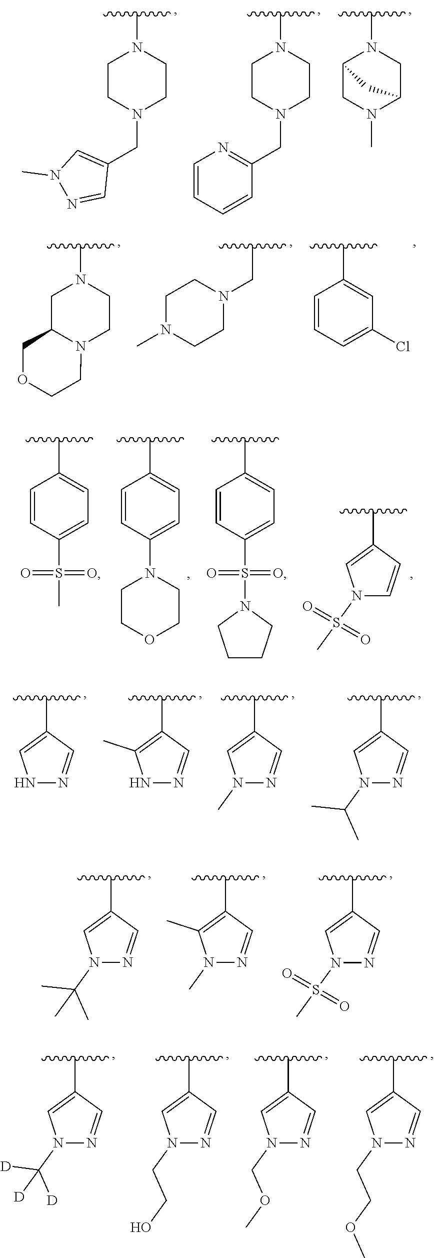

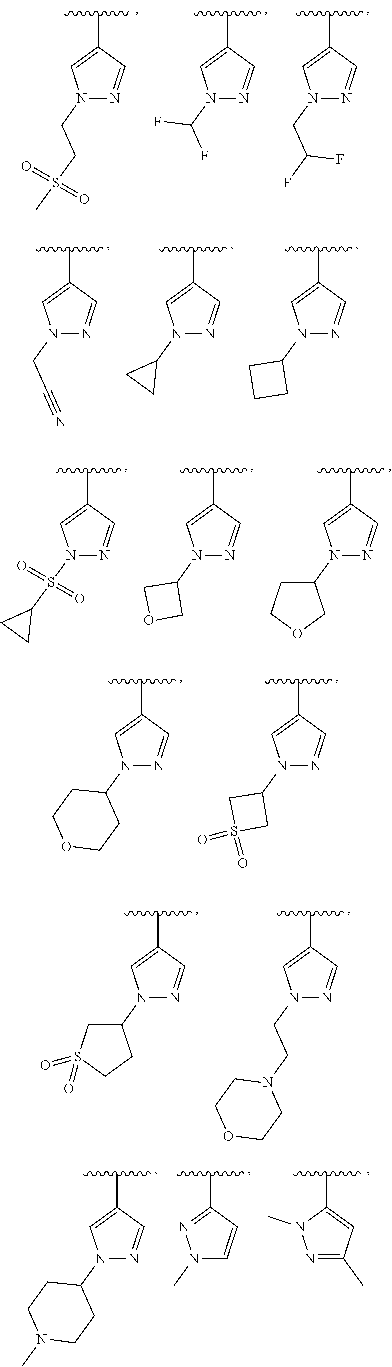

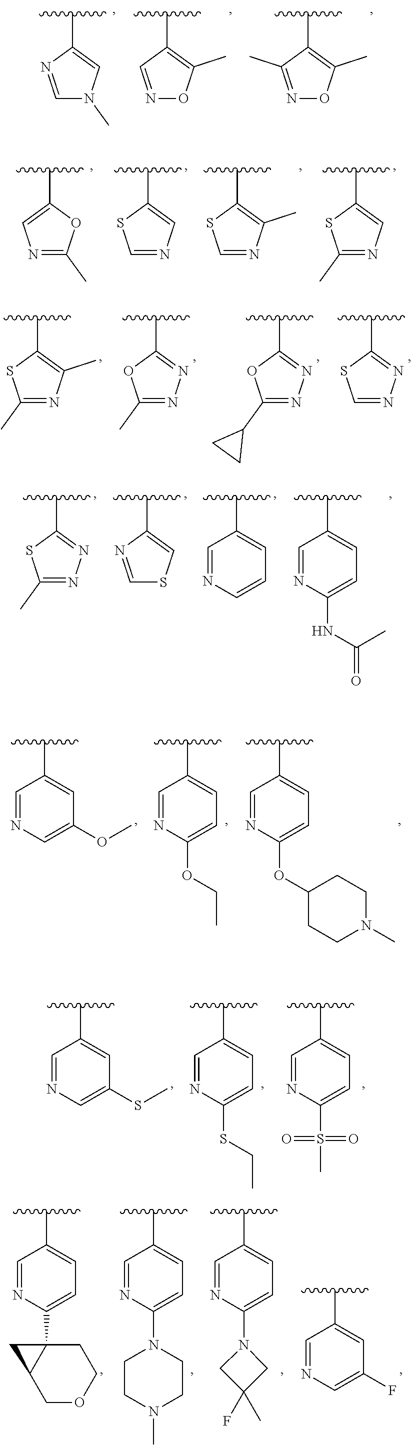

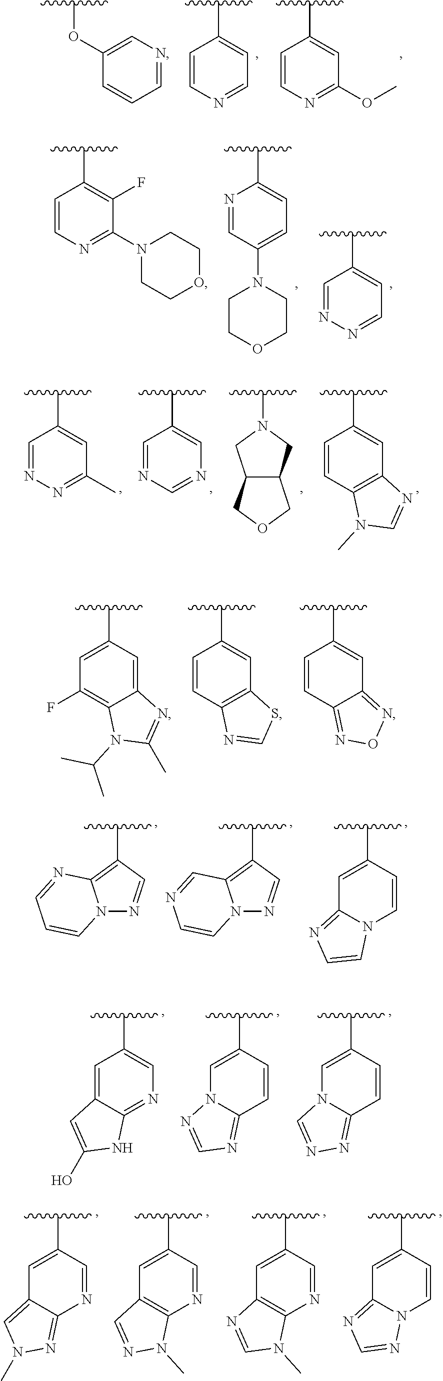

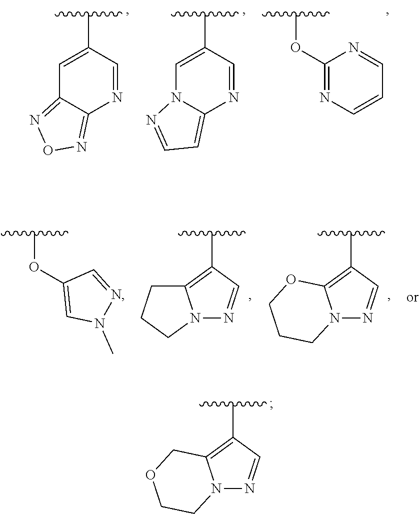

alternatively, ring B and three R^{8f} groups form the moiety

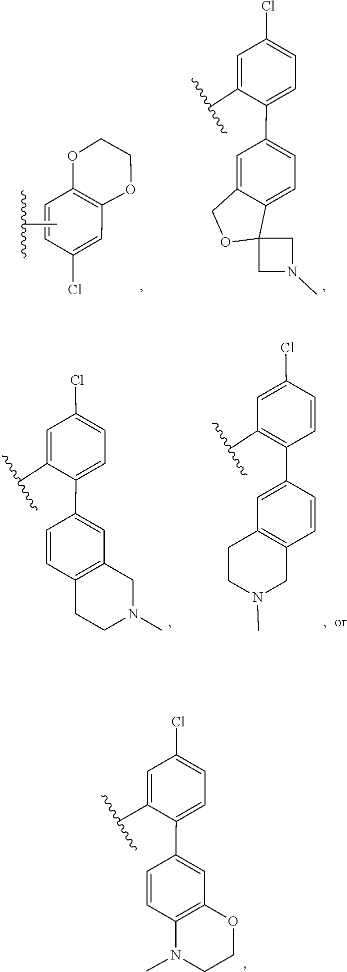

wherein the wavy line identifies attachment to the remainder of the molecule.

In some embodiments, the compound, or the pharmaceutically acceptable salt thereof, is the compound of Formula (I), (Ib), (Ib1), (Ic), (Ic1), (Id), or (Id1), wherein
R³ is

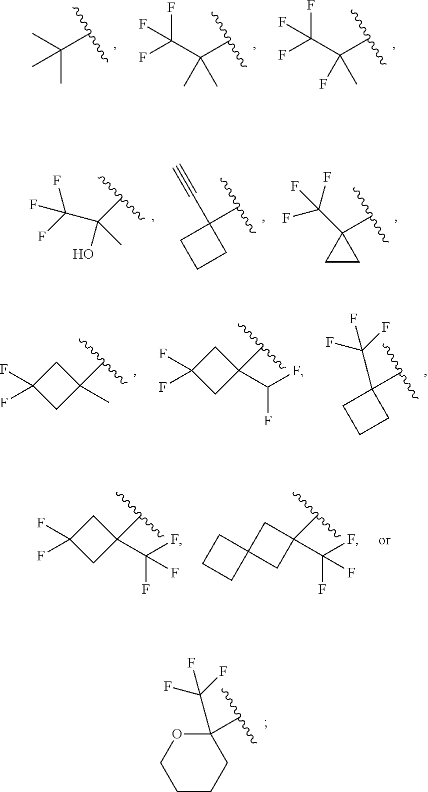

R^{4a} and R^{4b} are each H;
R^{4c} is ethyl;
alternatively, R^{4c} and R^{4a} together with the carbon and nitrogen to which each is attached combine to form a pyrrolidinyl, substituted with 0 to 2 fluoro;
R^{5a} and R^{5b} are each H;
R^{5c} is H, methyl, ethyl,

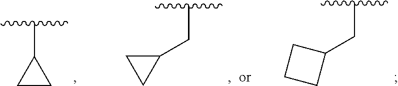

X⁶ is

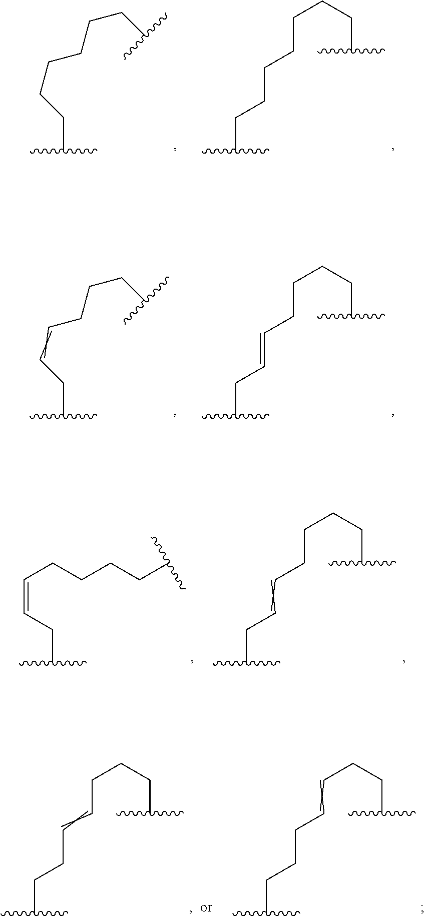

R^{6a} is H, methyl, ethyl, or

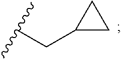 [SMILES: *CC1CC1]

R^{6b} is H;
R^{6d} is H, methyl, or ethyl;
R^{7a} and R^{7b} are each H;
R^{7c} is isobutyl,

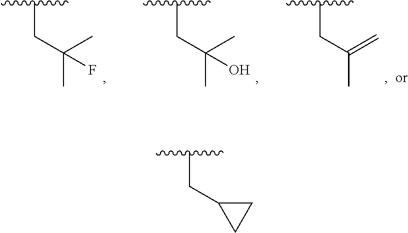

R^{8a} is H or methyl;
R^{8b}, R^{8d} and R^{8e} are each H;
ring B is phenyl, biphenyl, or pyrid-3-yl;
m8 is 1, 2, or 3; and
each R^{8f} is independently methyl, isopropyl, isobutyl, methoxy, ethoxy, fluoro, chloro, cyano,

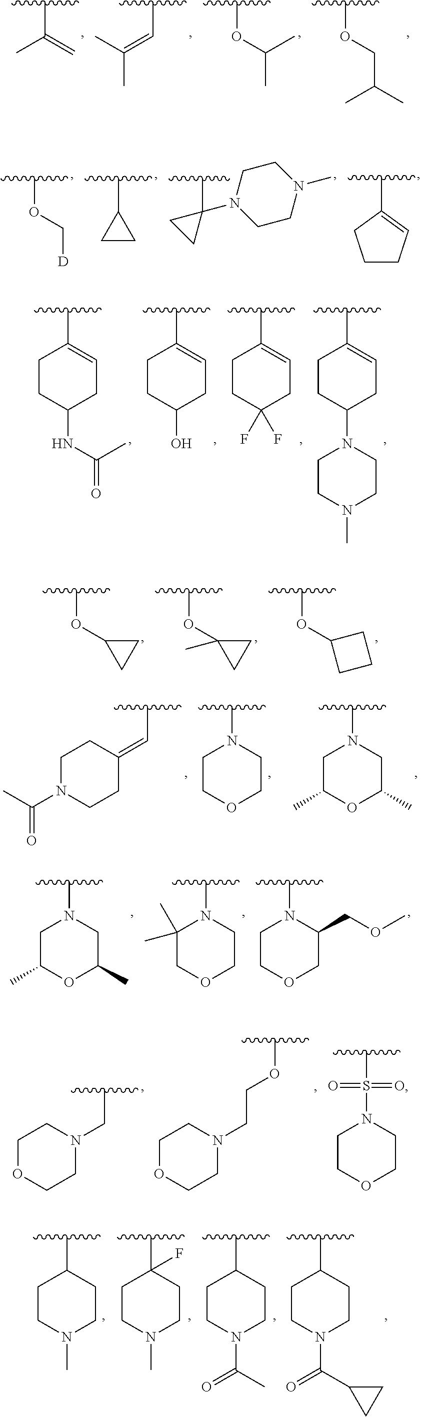

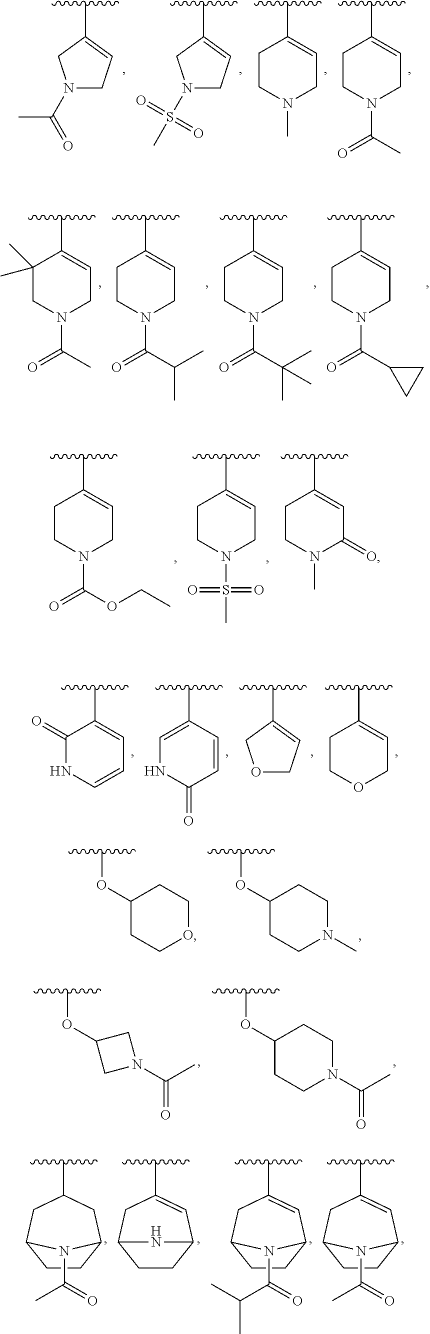

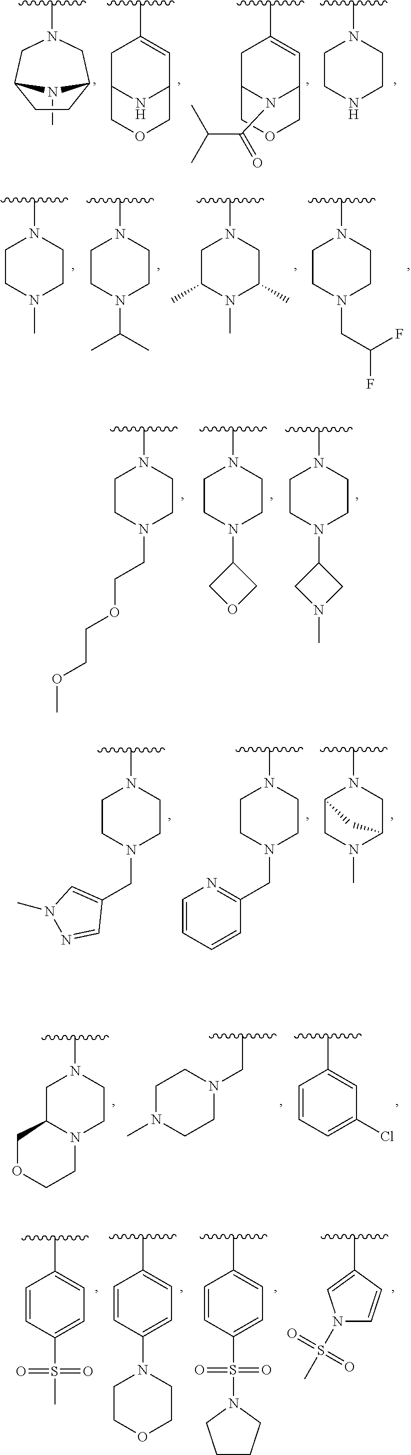

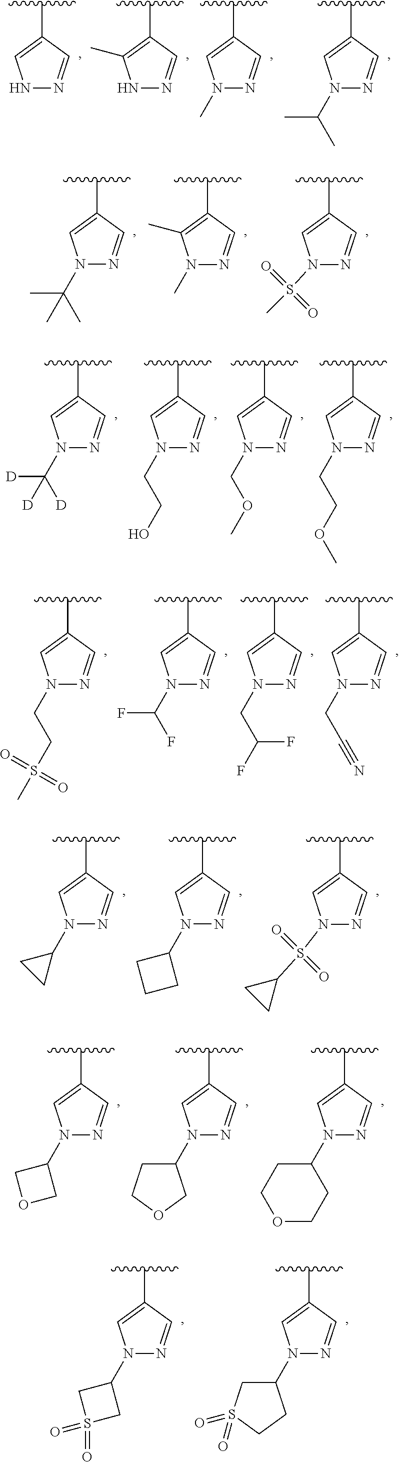

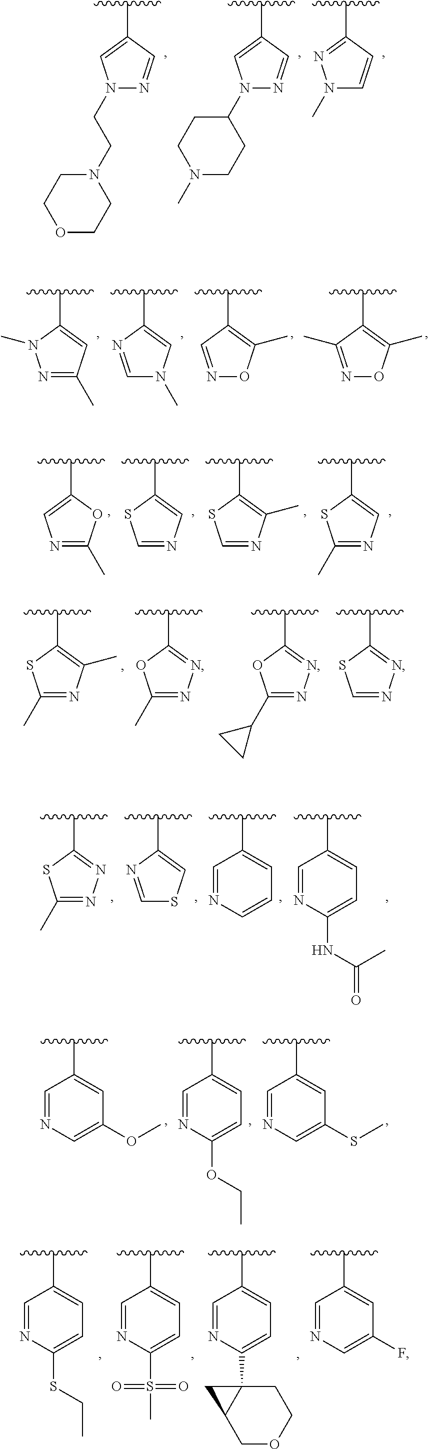

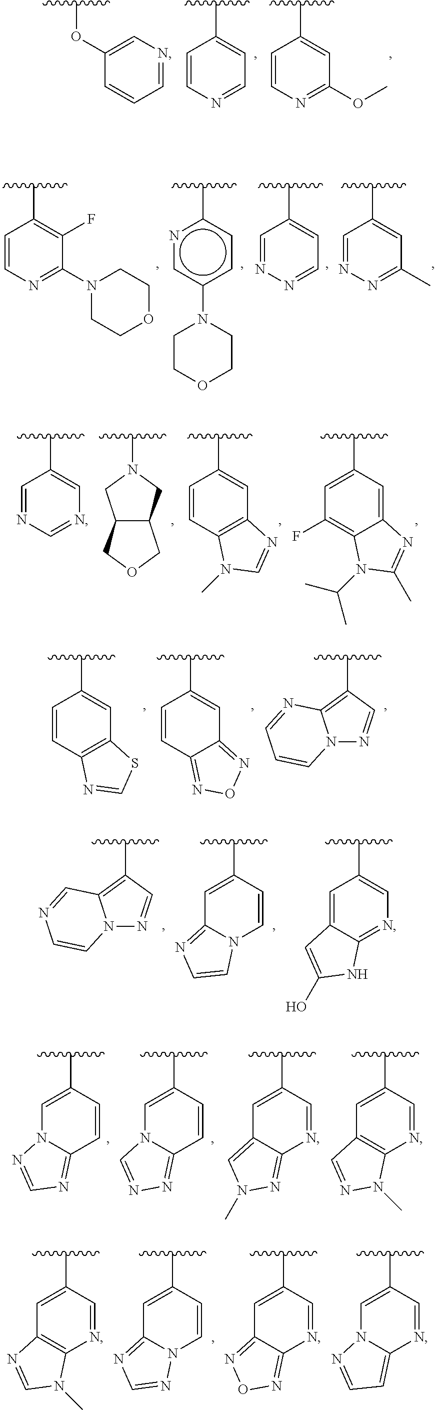

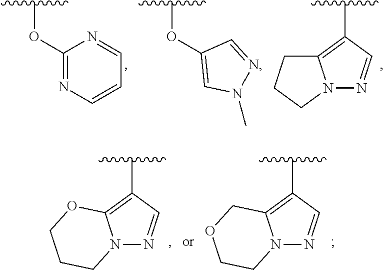

alternatively, ring B and three R^{8f} groups form the moiety

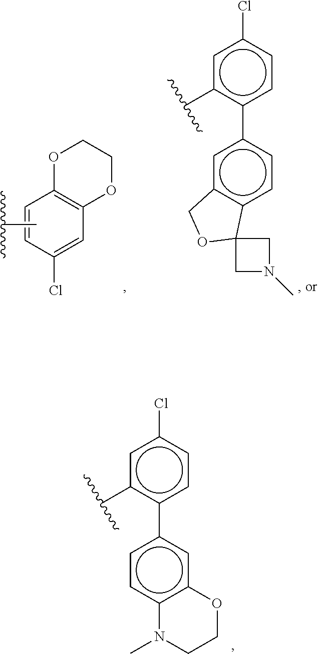

wherein the wavy line identifies attachment to the remainder of the molecule.

In some embodiments, the compound, or the pharmaceutically acceptable salt thereof, is the compound of Formula (I), (Ib), (Ib1), (Ic), (Ic1), (Id), or (Id1), wherein
R³ is

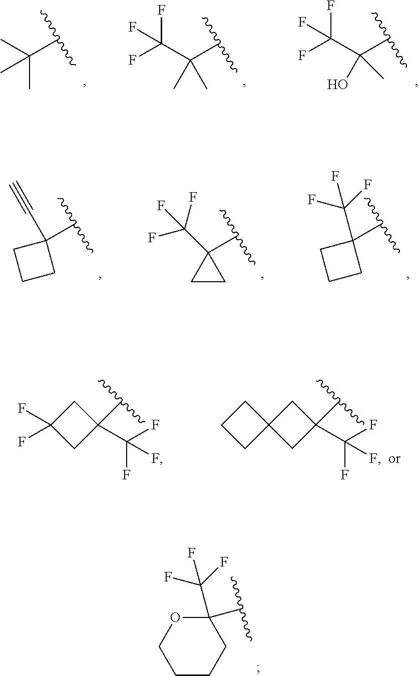

R^{4a} and R^{4b} are each H;
R^{4c} is ethyl;
alternatively, R^{4c} and R^{4a} together with the carbon and nitrogen to which each is attached combine to form a pyrrolidinyl, substituted with 0 to 2 fluoro;
R^{5a} and R^{5b} are each H;
R^{5c} is H, methyl, ethyl

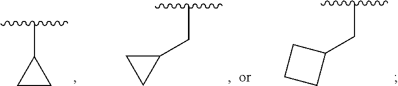

X⁶ is

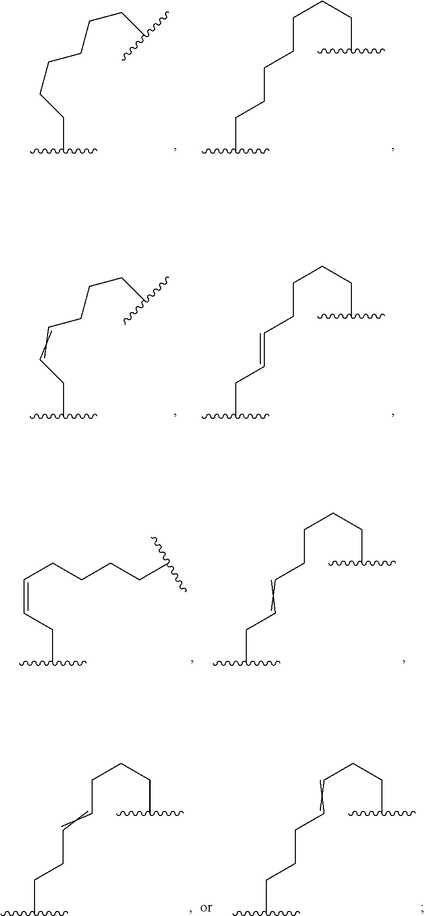

R^{6a} is H, methyl, ethyl, or

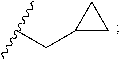 [SMILES: *CC1CC1]

R^{6b} is H;
R^{6d} is H or methyl;
R^{7a} and R^{7b} are each H;
R^{7c} is isobutyl,

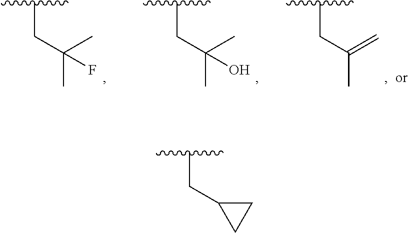

R^{8a} is H or methyl;
R^{8b}, R^{8d} and R^{8e} are each H;
ring B is phenyl or pyrid-3-yl;
m8 is 1, 2, or 3; and
each R^{8f} is independently methyl, isopropyl, isobutyl, methoxy, ethoxy, fluoro, chloro,

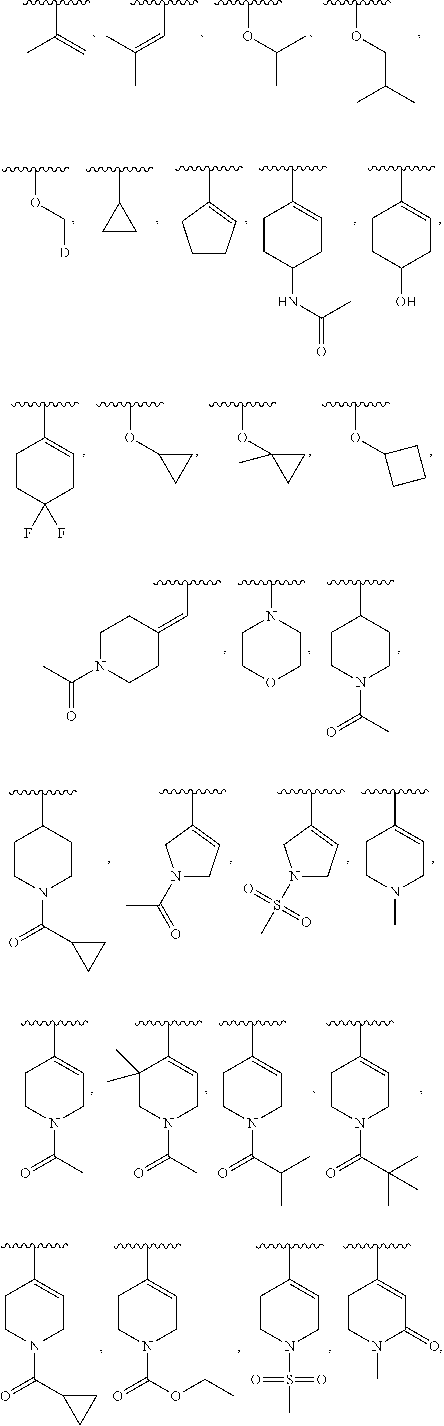

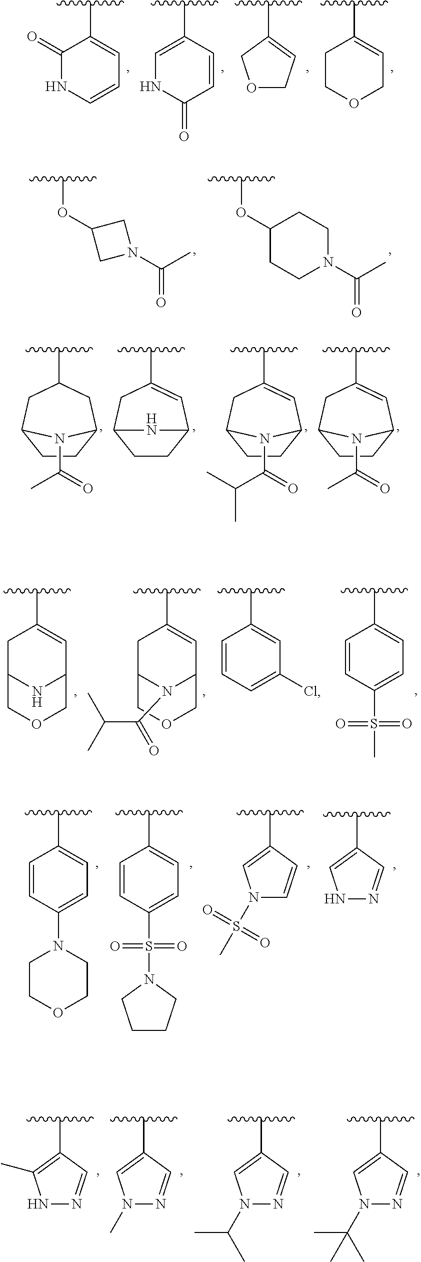

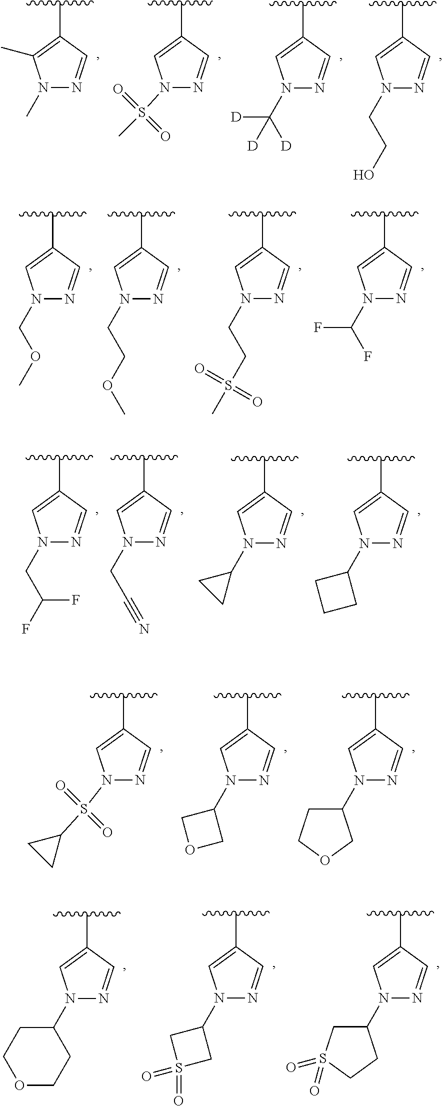

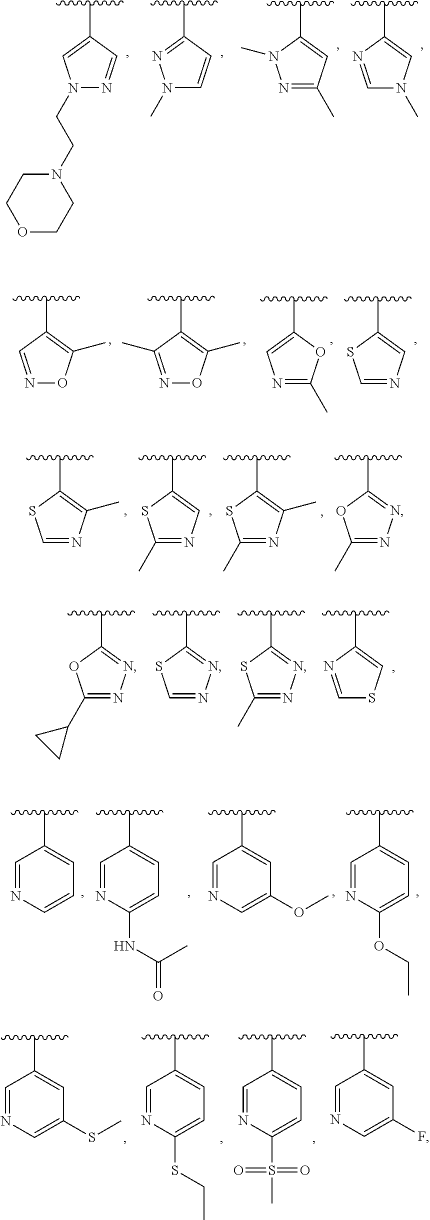

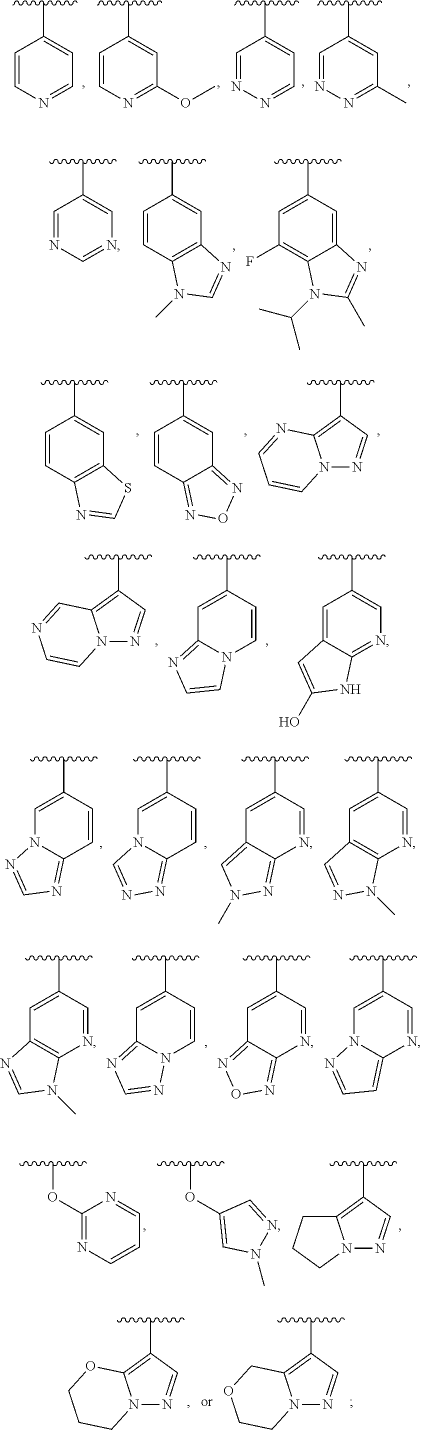

alternatively, ring B and three R^{8f} groups form the moiety

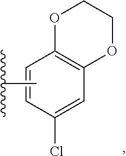

wherein the wavy line identifies attachment to the remainder of the molecule.

For the above embodiment, R³, R^{4a}, R^{4b}, R^{4c}, R^{5a}, R^{5b}, R^{5e}, X⁶, R^{6a}, R^{6b}, R^{6d}, R^{7a}, R^{7b}, R^{7c}, R^{8a}, R^{8b}, R^{8d}, R^{8e}, ring B, m8, and R^{8f} can each independently be as defined for any embodiment of Formula (I), (Ib), (Ib1), (Ic), (Ic1) (Id), or (Id1) as described herein.

In some embodiments, the compound, or the pharmaceutically acceptable salt thereof, is the compound of Formula (I), (Ib), (Ib1), (Ic), (Ic1) (Id), or (Id1) having the structure of any one of Examples 1-426. In some embodiments, the compound, or the pharmaceutically acceptable salt thereof, is the compound of Formula (I), (Ib), (Ib1), (Ic), (Ic1) (Id), or (Id1) having the structure of any one of Examples 1-389. In some embodiments, the compound, or the pharmaceutically acceptable salt thereof, is the compound of Formula (I), (Ib), (Ib1), (Ic), (Ic1) (Id), or (Id1) having the structure of any one of Examples 1-334. In some embodiments, the compound, or the pharmaceutically acceptable salt thereof, is the compound of Formula (I), (Ib), (Ib1), (Ic), (Ic1) (Id), or (Id1) having the structure of any one of Examples 1-50. In some embodiments, the compound, or the pharmaceutically acceptable salt thereof, is the compound of Formula (I), (Ib), (Ib1), (Ic), (Ic1) (Id), or (Id1) having the structure of any one of Examples 51-100. In some embodiments, the compound, or the pharmaceutically acceptable salt thereof, is the compound of Formula (I), (Ib), (Ib1), (Ic), (Ic1) (Id), or (Id1) having the structure of any one of Examples 101-150. In some embodiments, the compound, or the pharmaceutically acceptable salt thereof, is the compound of Formula (I), (Ib), (Ib1), (Ic), (Ic1) (Id), or (Id1) having the structure of any one of Examples 151-200. In some embodiments, the compound, or the pharmaceutically acceptable salt thereof, is the compound of Formula (I), (Ib), (Ib1), (Ic), (Ic1) (Id), or (Id1) having the structure of any one of Examples 201-250. In some embodiments, the compound, or the pharmaceutically acceptable salt thereof, is the compound of Formula (I), (Ib), (Ib1), (Ic), (Ic1) (Id), or (Id1) having the structure of any one of Examples 251-300. In some embodiments, the compound, or the pharmaceutically acceptable salt thereof, is the compound of Formula (I), (Ib), (Ib1), (Ic), (Ic1) (Id), or (Id1) having the structure of any one of Examples 301-334. In some embodiments, the compound, or the pharmaceutically acceptable salt thereof, is the compound of Formula (I), (Ib), (Ib1), (Ic), (Ic1) (Id), or (Id1) having the structure of any one of Examples 335-389. In some embodiments, the compound, or the pharmaceutically acceptable salt thereof, is the compound of Formula (I), (Ib), (Ib1), (Ic), (Ic1) (Id), or (Id1) having the structure of any one of Examples 390-426.

In some embodiments, the compound, or the pharmaceutically acceptable salt thereof, is the compound of Formula (I) having the structure

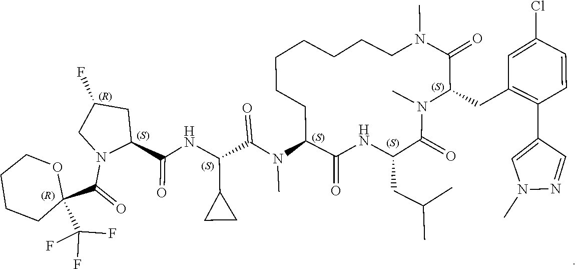

In some embodiments, the compound, or the pharmaceutically acceptable salt thereof, is the compound of Formula (I) having the structure

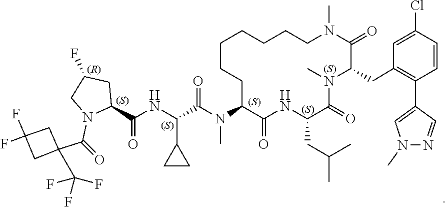

In some embodiments, the compound, or the pharmaceutically acceptable salt thereof, is the compound of Formula (I) having the structure

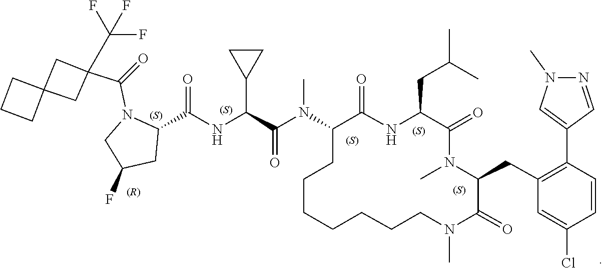

In some embodiments, the compound, or the pharmaceutically acceptable salt thereof, is the compound of Formula (I) having the structure

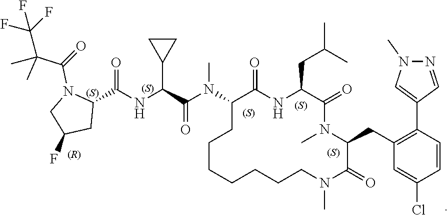

In some embodiments, the compound, or the pharmaceutically acceptable salt thereof, is the compound of Formula (I) having the structure

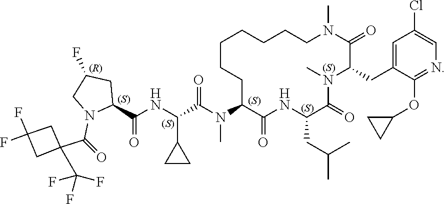

In some embodiments, the compound, or the pharmaceutically acceptable salt thereof, is the compound of Formula (I) having the structure

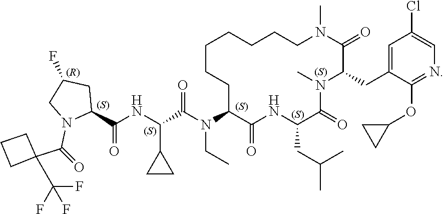

In some embodiments, the compound, or the pharmaceutically acceptable salt thereof, is the compound of Formula (I) having the structure

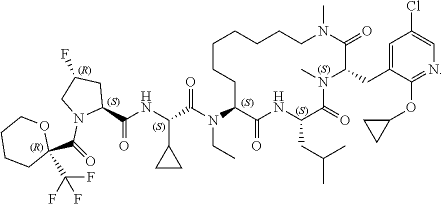

In some embodiments, the compound, or the pharmaceutically acceptable salt thereof, is the compound of Formula (I) having the structure

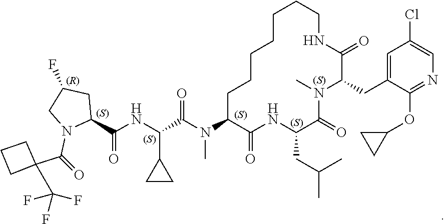

In some embodiments, the compound, or the pharmaceutically acceptable salt thereof, is the compound of Formula (I) having the structure

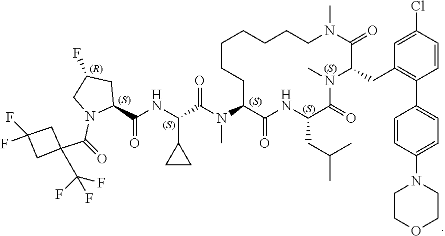

In some embodiments, the compound, or the pharmaceutically acceptable salt thereof, is the compound of Formula (I) having the structure

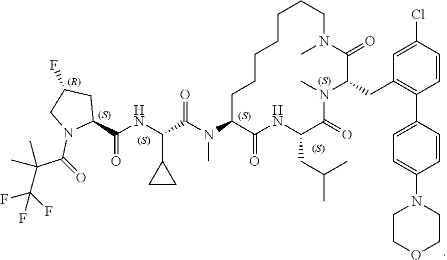

In some embodiments, the compound, or the pharmaceutically acceptable salt thereof, is the compound of Formula (I) having the structure

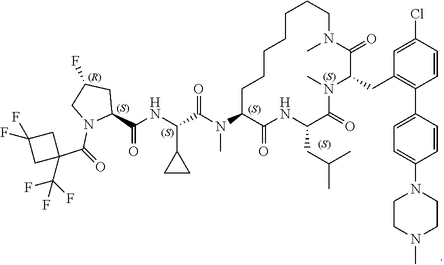

In some embodiments, the compound, or the pharmaceutically acceptable salt thereof, is the compound of Formula (I) having the structure

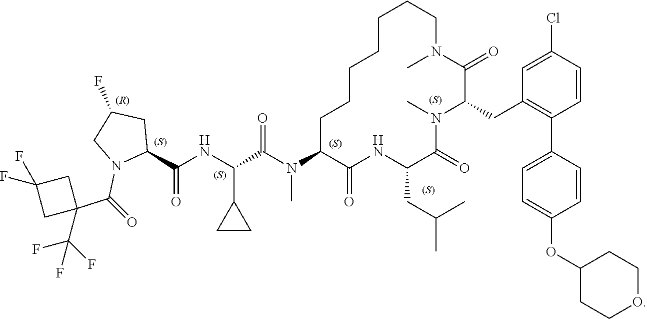

In some embodiments, the compound, or the pharmaceutically acceptable salt thereof, is the compound of Formula (I) having the structure

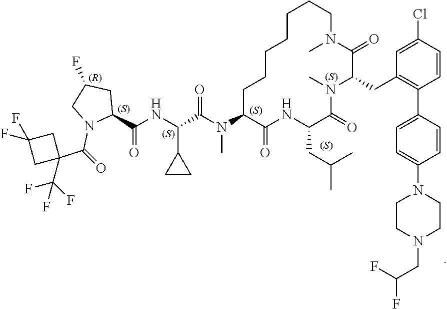

In some embodiments, the compound, or the pharmaceutically acceptable salt thereof, is the compound of Formula (I) having the structure

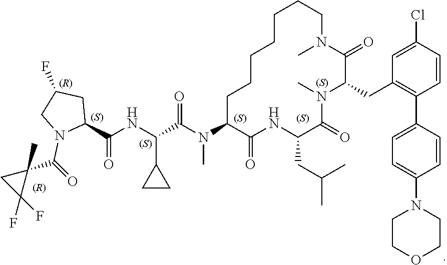

The present disclosure includes all tautomers and stereoisomers of the compounds described herein, either in admixture or in pure or substantially pure form. The compounds of Formula (I), (Ib), (Ib1), (Ic), (Ic1) (Id), or (Id1) can have asymmetric centers at one or more carbon atoms, and therefore compounds of Formula (I), (Ib), (Ib1), (Ic), (Ic1) (Id), or (Id1) can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, and tautomers are within the scope of the present disclosure. Compounds of Formula (I), (Ib), (Ib1), (Ic), (Ic1) (Id), or (Id1) can be prepared using diastereomers, enantiomers or racemic mixtures as starting materials. Furthermore, diastereomer and enantiomer products can be separated by chromatography, fractional crystallization or other methods known to those of skill in the art. Unless otherwise indicated, when a stereochemical depiction is shown, it is meant that the isomer with the depicted stereochemistry is present and substantially free of the other isomer(s). “Substantially free of” another isomer indicates at least an 80/20 ratio of the two isomers, more preferably 90/10, or 95/5 or more. When a structure includes a wavy bond attached to a double bond, this indicates E, Z, or a mixture of both isomers.

The compounds of Formula (I), (Ib), (Ib1), (Ic), (Ic1) (Id), or (Id1) can also be in the salt forms, such as acid or base salts of the compounds of Formula (I), (Ib), (Ib1), (Ic), (Ic1) (Id), or (Id1). Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically acceptable salts of the acidic compounds of Formula (I), (Ib), (Ib1), (Ic), (Ic1) (Id), or (Id1) are salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

Similarly acid addition salts, such as of mineral acids, organic carboxylic and organic sulfonic acids, e.g., hydrochloric acid, methanesulfonic acid, maleic acid, are also possible provided a basic group, such as pyridyl, constitutes part of the structure.

The neutral forms of the compounds can be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

The present disclosure also includes isotopically-labeled compounds of Formula (I), (Ib), (Ib1), (Ic), (Ic1) (Id), or (Id1), wherein one or more atoms are replaced by one or more atoms having specific atomic mass or mass numbers. Examples of isotopes that can be incorporated into compounds of Formula (I), (Ib), (Ib1), (Ic), (Ic1) (Id), or (Id1) include, but are not limited to, isotopes of hydrogen, carbon, nitrogen, oxygen, fluorine, sulfur, and chlorine (such as ²H, ³H, ¹³C, ¹⁴C, ¹⁵N, ¹⁸O, ¹⁷O, ¹⁸F, ³⁵S and ³⁶Cl). Isotopically-labeled compounds of Formula (I), (Ib), (Ib1), (Ic), (Ic1) (Id), or (Id1) can be useful in assays of the tissue distribution of the compounds and their prodrugs and metabolites; preferred isotopes for such assays include ³H and ¹⁴C. In addition, in certain circumstances substitution with heavier isotopes, such as deuterium (²H), can provide increased metabolic stability, which offers therapeutic advantages such as increased in vivo half-life or reduced dosage requirements. Isotopically-labeled compounds of Formula (I), (Ib), (Ib1), (Ic), (Ic1) (Id), or (Id1) can generally be prepared according to methods known in the art.

### IV. Compositions

The compounds of Formula (I), (Ib), (Ib1), (Ic), (Ic1) (Id), or (Id1) described herein are useful in the manufacture of a pharmaceutical composition or a medicament for modulating one or more cyclins (e.g. cyclin A, cyclin B, cycline E). In some embodiments, the present disclosure provides a pharmaceutical composition comprising a compound of the present disclosure, and a pharmaceutically acceptable excipient. In some embodiments, a pharmaceutical composition or medicament comprising one or more compounds of Formula (I), (Ib), (Ib1), (Ic), (Ic1) (Id), or (Id1) can be administered to a subject for the treatment of a cancer.

Pharmaceutical compositions or medicaments for use in the present disclosure can be formulated by standard techniques or methods well-known in the art of pharmacy using one or more physiologically acceptable carriers or excipients. Suitable pharmaceutical carriers are described herein and in, e.g., “Remington's Pharmaceutical Sciences” by E. W. Martin. Compounds of Formula (I), (Ib), (Ib1), (Ic), (Ic1) (Id), or (Id1) and their physiologically acceptable salts and solvates can be formulated for administration by any suitable route, including, but not limited to, orally, topically, nasally, rectally, pulmonary, parenterally (e.g., intravenously, subcutaneously, intramuscularly, etc.), and combinations thereof. In some embodiments, the compounds of Formula (I), (Ib), (Ib1), (Ic), (Ic1) (Id), or (Id1) is dissolved in a liquid, for example, water. The most suitable route of administration for a compound of Formula (I), (Ib), (Ib1), (Ic), (Ic1) (Id), or (Id1) in any given case will depend, in part, on the nature, severity, and optionally, and the stage of the cancer.

The pharmaceutical compositions or medicaments of the present disclosure can include a compound of Formula (I), (Ib), (Ib1), (Ic), (Ic1) (Id), or (Id1) with as an active ingredient and a pharmaceutically acceptable carrier and/or excipient or diluent. Any carrier and/or excipient suitable for the form of preparation desired for administration is contemplated for use with the compounds of Formula (I), (Ib), (Ib1), (Ic), (Ic1) (Id), or (Id1) disclosed herein.

In some embodiments, the pharmaceutical compositions or medicaments described herein are suitable for systemic administration. Systemic administration includes enteral administration (e.g., absorption of the compound through the gastrointestinal tract) or parenteral administration (e.g., injection, infusion, or implantation). In some embodiments, the pharmaceutical compositions or medicaments can be administered via a syringe or intravenously. In preferred embodiments, the pharmaceutical compositions or medicaments are injected subcutaneously.

For oral administration, a pharmaceutical composition or a medicament can take the form of, e.g., a tablet or a capsule prepared by conventional means with a pharmaceutically acceptable excipient. Preferred are tablets and gelatin capsules comprising the active ingredient(s), together with (a) diluents or fillers, e.g., lactose, dextrose, sucrose, mannitol, sorbitol, cellulose (e.g., ethyl cellulose, microcrystalline cellulose), glycine, pectin, polyacrylates and/or calcium hydrogen phosphate, calcium sulfate, (b) lubricants, e.g., silica, anhydrous colloidal silica, talcum, stearic acid, its magnesium or calcium salt (e.g., magnesium stearate or calcium stearate), metallic stearates, colloidal silicon dioxide, hydrogenated vegetable oil, corn starch, sodium benzoate, sodium acetate and/or polyethyleneglycol; for tablets also (c) binders, e.g., magnesium aluminum silicate, starch paste, gelatin, tragacanth, methylcellulose, sodium carboxymethylcellulose, polyvinylpyrrolidone and/or hydroxypropyl methylcellulose; if desired (d) disintegrants, e.g., starches (e.g., potato starch or sodium starch), glycolate, agar, alginic acid or its sodium salt, or effervescent mixtures; (e) wetting agents, e.g., sodium lauryl sulfate, and/or (f) absorbents, colorants, flavors and sweeteners. In some embodiments, the tablet contains a mixture of hydroxypropyl methylcellulose, polyethyleneglycol 6000 and titatium dioxide. Tablets can be either film coated or enteric coated according to methods known in the art.

Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

Typical formulations for topical administration include creams, ointments, sprays, lotions, and patches. The pharmaceutical composition can, however, be formulated for any type of administration, e.g., intradermal, subdermal, intravenous, intramuscular, intranasal, intracerebral, intratracheal, intraarterial, intraperitoneal, intravesical, intrapleural, intracoronary or intratumoral injection, with a syringe or other devices. Formulation for administration by inhalation (e.g., aerosol), or for oral, rectal, or vaginal administration is also contemplated.

Pharmaceutical compositions for pulmonary administration include, but are not limited to, dry powder compositions consisting of the powder of a compound described herein, or a salt thereof, and the powder of a suitable carrier and/or lubricant. The compositions for pulmonary administration can be inhaled from any suitable dry powder inhaler device known to a person skilled in the art. In certain instances, the compositions can be conveniently delivered in the form of an aerosol spray from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound(s) and a suitable powder base, for example, lactose or starch.

The compounds of Formula (I), (Ib), (Ib1), (Ic), (Ic1) (Id), or (Id1) can also be formulated in rectal compositions, for example, suppositories or retention enemas, for example, containing conventional suppository bases, for example, cocoa butter or other glycerides.

The compounds of Formula (I), (Ib), (Ib1), (Ic), (Ic1) (Id), or (Id1) set forth herein can be formulated for parenteral administration by injection, for example by bolus injection. Formulations for injection can be presented in unit dosage form, for example, in ampoules or in multi-dose containers, with an added preservative. Injectable compositions are preferably aqueous isotonic solutions or suspensions, and suppositories are preferably prepared from fatty emulsions or suspensions. The compositions can be sterilized and/or contain adjuvants, such as preserving, stabilizing, wetting or emulsifying agents, solution promoters, salts for regulating the osmotic pressure and/or buffers. Alternatively, the compound(s) can be in powder form for reconstitution with a suitable vehicle, for example, sterile pyrogen-free water, before use. In addition, they may also contain other therapeutically valuable substances. The compositions are prepared according to conventional mixing, granulating or coating methods, respectively, and contain about 0.1 to 75%, preferably about 1 to 50%, of the compound(s).

In some embodiments, the compositions described herein are prepared with a polysaccharide such as chitosan or derivatives thereof (e.g., chitosan succinate, chitosan phthalate, etc.), pectin and derivatives thereof (e.g., amidated pectin, calcium pectinate, etc.), chondroitin and derivatives thereof (e.g., chondroitin sulfate), and alginates.

In some embodiments, the compositions described herein further include a pharmaceutical surfactant. In other embodiments, the compositions further include a cryoprotectant. Non-limiting examples of cryoprotectants include glucose, sucrose, trehalose, lactose, sodium glutamate, PVP, cyclodextrin, 2-hydroxypropyl-β-cyclodextrin (HPI3CD) glycerol, maltose, mannitol, saccharose, and mixtures thereof.

### V. Methods

The present disclosure contemplates the use of the compounds of Formula (I), (Ib), (Ib1), (Ic), (Ic1) (Id), or (Id1) described herein in the treatment or prevention of diseases or disorders modulated, at least in part, by one or more cyclins. In some embodiments, the cyclin mediated disease is a proliferative condition or disorder, including cancer. In some embodiments, the present disclosure provides a method of treating a cancer mediated at least in part by cyclin activity, the method comprising administering to a subject in need there of, a therapeutically effective amount of a compound of the present disclosure, or a pharmaceutically acceptable salt thereof, or a pharmaceutical composition of the present disclosure, thereby treating the cancer.

In some embodiments, provided herein are compounds of Formula (I), (Ib), (Ib1), (Ic), (Ic1) (Id), or (Id1) for use in therapy.

The present disclosure contemplates the use of the compounds of Formula (I), (Ib), (Ib1), (Ic), (Ic1) (Id), or (Id1) described herein in the treatment or prevention of diseases or disorders modulated, at least in part, by cyclin A. In some embodiments, the cyclin A mediated disease is a proliferative condition or disorder, including cancer. In some embodiments, the present disclosure provides a method of treating a cancer mediated at least in part by cyclin A, the method comprising administering to a subject in need there of, a therapeutically effective amount of a compound of the present disclosure, or a pharmaceutically acceptable salt thereof, or a pharmaceutical composition of the present disclosure, thereby treating the cancer.

In some embodiments, provided herein are methods of treating a proliferative condition or disorder mediated at least in part by cyclin A comprising administering a compound of Formula (I), (Ib), (Ib1), (Ic), (Ic1) (Id), or (Id1) described herein.

In some embodiments, provided herein are compounds of Formula (I), (Ib), (Ib1), (Ic), (Ic1) (Id), or (Id1) for use in a method for treating a proliferative condition or disorder mediated at least in part by cyclin A.

In some embodiments, provided herein are uses of compounds of Formula (I), (Ib), (Ib1), (Ic), (Ic1) (Id), or (Id1) for the manufacture of a medicament for the treatment of a proliferative condition or disorder mediated at least in part by cyclin A.

The present disclosure contemplates the use of the compounds of Formula (I), (Ib), (Ib1), (Ic), (Ic1) (Id), or (Id1) described herein in the treatment or prevention of diseases or disorders modulated, at least in part, by cyclin B. In some embodiments, the cyclin B mediated disease is a proliferative condition or disorder, including cancer. In some embodiments, the present disclosure provides a method of treating a cancer mediated at least in part by cyclin B, the method comprising administering to a subject in need there of, a therapeutically effective amount of a compound of the present disclosure, or a pharmaceutically acceptable salt thereof, or a pharmaceutical composition of the present disclosure, thereby treating the cancer.

In some embodiments, provided herein are methods of treating a proliferative condition or disorder mediated at least in part by cyclin B comprising administering a compound of Formula (I), (Ib), (Ib1), (Ic), (Ic1) (Id), or (Id1) described herein.

In some embodiments, provided herein are compounds of Formula (I), (Ib), (Ib1), (Ic), (Ic1) (Id), or (Id1) for use in a method for treating a proliferative condition or disorder mediated at least in part by cyclin B.

In some embodiments, provided herein are uses of compounds of Formula (I), (Ib), (Ib1), (Ic), (Ic1) (Id), or (Id1) for the manufacture of a medicament for the treatment of a proliferative condition or disorder mediated at least in part by cyclin B.

The present disclosure contemplates the use of the compounds of Formula (I), (Ib), (Ib1), (Ic), (Ic1) (Id), or (Id1) described herein in the treatment or prevention of diseases or disorders modulated, at least in part, by cyclin E. In some embodiments, the cyclin E mediated disease is a proliferative condition or disorder, including cancer. In some embodiments, the present disclosure provides a method of treating a cancer mediated at least in part by cyclin E, the method comprising administering to a subject in need there of, a therapeutically effective amount of a compound of the present disclosure, or a pharmaceutically acceptable salt thereof, or a pharmaceutical composition of the present disclosure, thereby treating the cancer.

In some embodiments, provided herein are methods of treating a proliferative condition or disorder mediated at least in part by cyclin E comprising administering a compound of Formula (I), (Ib), (Ib1), (Ic), (Ic1) (Id), or (Id1) described herein.

In some embodiments, provided herein are compounds of Formula (I), (Ib), (Ib1), (Ic), (Ic1) (Id), or (Id1) for use in a method for treating a proliferative condition or disorder mediated at least in part by cyclin E.

In some embodiments, provided herein are uses of compounds of Formula (I), (Ib), (Ib1), (Ic), (Ic1) (Id), or (Id1) for the manufacture of a medicament for the treatment of a proliferative condition or disorder mediated at least in part by cyclin E.

In some embodiments, the compounds of Formula (I), (Ib), (Ib1), (Ic), (Ic1) (Id), or (Id1) described herein can be used to treat or prevent a proliferative condition or disorder, including a cancer, for example, cancer of the uterus, cervix, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, bile ducts, heart, lung (e.g., non-small-cell lung carcinoma, small cell lung cancer), pancreas, salivary gland, adrenal gland, thyroid, brain, ganglia, central nervous system (CNS) and peripheral nervous system (PNS), and cancers of the hematopoietic system and the immune system (e.g., spleen or thymus).

The present disclosure also provides methods of treating or preventing other cancer-related diseases, disorders or conditions, including, for example, virus-induced cancers (e.g., epithelial cell cancers, endothelial cell cancers, squamous cell carcinomas and papillomavirus), adenocarcinomas, lymphomas, carcinomas, melanomas, leukemias, myelomas, sarcomas, teratocarcinomas, chemically-induced cancers, metastasis, and angiogenesis.

In some embodiments, the tumor or cancer is colon cancer, ovarian cancer, breast cancer, melanoma, lung cancer, glioblastoma, or leukemia.

In some embodiments, the tumor or cancer is small cell lung cancer (SCLC).

The use of the term(s) cancer-related diseases, disorders and conditions is meant to refer broadly to conditions that are associated, directly or indirectly, with cancer, and includes, e.g., angiogenesis and precancerous conditions such as dysplasia.

In some embodiments, the cancer is a blood cancer (e.g., leukemia, lymphoma, multiple myeloma).

In some embodiments, the leukemia is acute lymphocytic leukemia, chronic lymphocytic leukemia, acute myelogenous leukemia, chronic myelogenous leukemia, or hairy cell leukemia.

In some embodiments, the lymphoma is non-Hodgkin's lymphoma, Hodgkin's lymphoma, B-cell lymphoma, or Burkitt's lymphoma.

In some embodiments, the cancer is an Rb mutated cancer. In some embodiments, the cancer has a mutation in the Rb/E2F pathway.

### VI. Administration

The present disclosure contemplates the administration of compounds of Formula (I), (Ib), (Ib1), (Ic), (Ic1) (Id), or (Id1) and compositions thereof, in any appropriate manner. Suitable routes of administration include oral, parenteral (e.g., intramuscular, intravenous, subcutaneous (e.g., injection or implant), intraperitoneal, intracisternal, intraarticular, intraperitoneal, intracerebral (intraparenchymal) and intracerebroventricular), nasal, vaginal, sublingual, intraocular, rectal, topical (e.g., transdermal), buccal and inhalation.

Pharmaceutical compositions comprising compounds of Formula (I), (Ib), (Ib1), (Ic), (Ic1) (Id), or (Id1) are preferably in unit dosage form. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Compounds of Formula (I), (Ib), (Ib1), (Ic), (Ic1) (Id), or (Id1) or pharmaceutical compositions or medicaments thereof can be administered to a subject diagnosed or suspected of having a disease or disorder mediated at least in part by cyclin A in an amount sufficient to elicit an effective therapeutic response in the subject.

The dosage of compounds administered is dependent on a variety of factors including the subject's body weight, age, individual condition, and/or on the form of administration. The size of the dose will also be determined by the existence, nature, and extent of any adverse effects that accompany the administration of a particular compound in a particular subject. Typically, a dosage of the active compounds is a dosage that is sufficient to achieve the desired effect. Optimal dosing schedules can be calculated from measurements of compound accumulation in the body of a subject. In general, dosage can be given once or more daily, weekly, or monthly. Persons of ordinary skill in the art can easily determine optimum dosages, dosing methodologies, and repetition rates.

In some embodiments, a unit dosage for oral administration of a compound of Formula (I), (Ib), (Ib1), (Ic), (Ic1) (Id), or (Id1) described herein to a subject (e.g., a human) of about 50 to about 70 kg may contain between about 1 and about 5,000 mg, about 1 and about 3,000 mg, about 1 and about 2,000 mg, or about 1 to about 1,000 mg of the compound(s).

In some embodiments, a unit dosage for subcutaneous administration of a compound of Formula (I), (Ib), (Ib1), (Ic), (Ic1) (Id), or (Id1) described herein to a subject (e.g., human) of about 50 to about 70 kg may contain between about 0.1 and about 500 mg, about 0.5 and about 300 mg, about 0.5 and about 200 mg, about 0.5 and about 100 mg, or about 0.5 and about 50 mg.

The dose can be administered once per day or divided into sub-doses and administered in multiple doses, e.g., twice, three times, or four times per day. However, as will be appreciated by a skilled artisan, depending on the route of administration different amounts can be administered at different times.

In some embodiments, the compounds are administered for about 1 to 31 days, or for about 1 to 12 months. In some embodiments, the compounds are administered for one or more weeks, e.g., 1, 2, 3, 4, 5, 6, 7, 8, 9, 10, 11, 12, 13, 14, or more weeks. In some embodiments, the compounds are administered for one or more months, e.g., 1, 2, 3, 4, 5, 6, 7, 8, 9, 10, 11, 12, or more months.

Optimum dosages, toxicity, and therapeutic efficacy of such compounds may vary depending on the relative potency of individual compounds and can be determined by standard pharmaceutical procedures in experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side-effects can be used, care should be taken to design a delivery system that targets such compounds to the affected site to minimize potential damage to normal cells and, thereby, reduce side-effects.

The dosage of a pharmaceutical composition or medicament of the present disclosure can be monitored and adjusted throughout treatment, depending on severity of symptoms, frequency of recurrence, and/or the physiological response to the therapeutic regimen. Those of skill in the art commonly engage in such adjustments in therapeutic regimens.

Single or multiple administrations of the pharmaceutical compositions or medicaments can be administered depending on the dosage and frequency as required and tolerated by the patient. In any event, the composition or medicament should provide a sufficient quantity of the compounds of the disclosure to effectively treat the patient. Generally, when treating cancer, the dose is sufficient to stop tumor growth or cause tumor regression without producing unacceptable toxicity or side-effects to the patient.

### VII. Intermediates

In some embodiments, the present disclosure provides intermediates useful in the preparation of compounds of Formula (I). Certain intermediates useful in the preparation of a compound of Formula (I) can be found, for example, in the Examples section of the current disclosure.

In some embodiments, the intermediate is an External Building Block described herein. In some embodiments the intermediate is a compound produced in one of Methods A-D or Methods 1-14 for any one of the compounds exemplified herein. In some embodiments, the intermediate is one of Int. 1-438. In some embodiments, the intermediate is one of Int. 1-405. In some embodiments, the intermediate is one of Int. 1-154.

In some embodiments, the intermediate is Int. 7

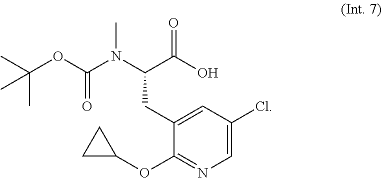 [SMILES: CN(C(=O)OC(C)(C)C)[C@@H](Cc1cc(Cl)cnc1OC1CC1)C(=O)O]

In some embodiments, the intermediate is Int. 30

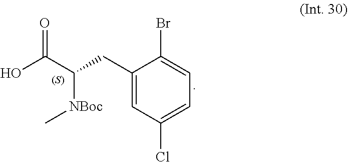

In some embodiments, the intermediate is Int. 142

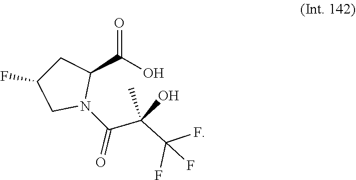 [SMILES: C[C@@](O)(C(=O)N1C[C@H](F)C[C@H]1C(=O)O)C(F)(F)F]

In some embodiments, the intermediate is a combination of one or more convalently linked External Building Blocks.

### VIII. Kits

The present disclosure contemplates kits comprising a compound of Formula (I), (Ib), (Ib1), (Ic), (Ic1) (Id), or (Id1) described herein described herein, and pharmaceutical compositions thereof. The kits are generally in the form of a physical structure housing various components, as described below, and can be utilized, for example, in practicing the methods described above.

A kit can include one or more of the compounds disclosed herein (provided in, e.g., a sterile container), which may be in the form of a pharmaceutical composition suitable for administration to a subject. The compounds described herein can be provided in a form that is ready for use (e.g., a tablet, capsule, syringe) or in a form requiring, for example, reconstitution or dilution (e.g., a powder) prior to administration. When the compounds described herein are in a form that needs to be reconstituted or diluted by a user, the kit may also include diluents (e.g., sterile water), buffers, pharmaceutically acceptable excipients, and the like, packaged with or separately from the compounds described herein. Each component of the kit can be enclosed within an individual container, and all of the various containers can be within a single package. A kit of the present disclosure can be designed for conditions necessary to properly maintain the components housed therein (e.g., refrigeration or freezing).

A kit may contain a label or packaging insert including identifying information for the components therein and instructions for their use (e.g., dosing parameters, clinical pharmacology of the active ingredient(s), including mechanism of action, pharmacokinetics and pharmacodynamics, adverse effects, contraindications, etc.). Labels or inserts can include manufacturer information such as lot numbers and expiration dates. The label or packaging insert may be, e.g., integrated into the physical structure housing the components, contained separately within the physical structure, or affixed to a component of the kit (e.g., an ampule, tube or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via the internet, are provided.

### IX. EXAMPLES

The following examples illustrate how various intermediates and exemplary compounds of Formula (I) are prepared. The following examples are offered to illustrate, but not to limit the current disclosure.

### A. External Building Blocks

The compounds of Formula (I) described herein are prepared by covalently linking the external building blocks described in this section. The external building blocks of the present disclosure are identified in Table 1, below, by intermediate number (INT #), IUPAC name, and CAS number, if known. For those without a CAS number, an experimental write-up is provided herein. The order and details related to covalently linking these external building blocks are described in another section.

TABLE 1
External Building Blocks of the Present Disclosure

| | | CAS Number, |
|---|---|---|
| INT # | IUPAC | if known |
| Int. 1 | hex-5-en-1-amine | 34825-70-2 |
| Int. 2 | 4-Penten-1-amine | 22537-07-1 |
| Int. 3 | N-Methylhex-5-en-1-amine | 55863-02-0 |
| Int. 4 | N-Methyl-4-penten-1-amine | 5831-72-1 |
| Int. 5 | 3-Buten-1-amine | 2524-49-4 |
| Int. 6 | (S)-2-((tert-butoxycarbonyl)(methyl)amino)-3- | |
| | (2,5-dichlorophenyl)propanoic acid | |
| Int. 7 | (S)-2-((tert-butoxycarbonyl)(methyl)amino)-3-(5- | |
| | chloro-2-cyclopropoxypyridin-3-yl)propanoic acid | |
| Int. 8 | (S)-2-((tert-butoxycarbonyl)(methyl)amino)-3-(5- | |
| | chloro-2-(5-methyl-1,3,4-oxadiazol-2- | |
| | yl)phenyl)propanoic acid | |
| Int. 9 | (S)-2-((tert-butoxycarbonyl)(methyl)amino)-3-(2- | |
| | cyclopropoxy-5-fluoropyridin-3-yl)propanoic acid | |
| Int. 10 | (S)-2-((tert-butoxycarbonyl)(methyl)amino)-3-(5- | |
| | chloro-2-isopropoxypyridin-3-yl)propanoic acid | |
| Int. 11 | (S)-2-((tert-butoxycarbonyl)(methyl)amino)-3-(5- | |
| | chloro-2-(pyrimidin-2-yloxy)phenyl)propanoic | |
| | acid | |
| Int. 12 | (S)-2-((tert-butoxycarbonyl)(methyl)amino)-3-(5- | |
| | chloro-2-cyclobutoxypyridin-3-yl)propanoic acid | |
| Int. 13 | (S)-2-((tert-butoxycarbonyl)(methyl)amino)-3-(5- | |
| | chloro-2-methoxypyridin-3-yl)propanoic acid | |
| Int. 14 | (S)-2-((tert-butoxycarbonyl)(methyl)amino)-3-(5- | |
| | chloro-2-ethoxypyridin-3-yl)propanoic acid | |
| Int. 15 | (S)-2-((tert-butoxycarbonyl)(methyl)amino)-3-(5- | |
| | chloro-2-isobutoxypyridin-3-yl)propanoic acid | |
| Int. 16 | (S)-2-((tert-butoxycarbonyl)(methyl)amino)-3-(5- | |
| | chloro-2-(5-cyclopropyl-1,3,4-oxadiazol-2- | |
| | yl)phenyl)propanoic acid | |
| Int. 17 | (S)-3-(2-((1-acetylpiperidin-4-yl)oxy)-5- | |
| | chlorophenyl)-2-((tert- | |
| | butoxycarbonyl)(methyl)amino)propanoic acid | |
| Int. 18 | (S)-2-((tert-butoxycarbonyl)(methyl)amino)-3-(5- | |
| | fluoro-2-(1-methyl-1H-pyrazol-4-yl)pyridin-3- | |
| | yl)propanoic acid | |
| Int. 19 | (S)-2-((tert-butoxycarbonyl)(methyl)amino)-3-(5- | |
| | chloro-2-(1-methyl-1H-pyrazol-4-yl)pyridin-3- | |
| | yl)propanoic acid | |
| Int. 20 | (S)-2-((tert-butoxycarbonyl)(methyl)amino)-3-(5- | |
| | chloro-2-(1- | |
| | methylcyclopropoxy)phenyl)propanoic acid | |
| Int. 21 | (S)-2-((((9H-fluoren-9- | 146549-21-5 |
| | yl)methoxy)carbonyl)amino)pent-4-enoic acid | |
| Int. 22 | (S)-3-(1′-acetyl-5-chloro-1′,2′,3′,6′-tetrahydro- | |
| | [2,4′-bipyridin]-3-yl)-2-((tert- | |
| | butoxycarbonyl)(methyl)amino)propanoic acid | |
| Int. 23 | (S)-2-((tert-butoxycarbonyl)(methyl)amino)-3-(5- | |
| | chloro-2-cyclopropoxyphenyl)propanoic acid | |
| Int. 24 | (S)-2-((tert-butoxycarbonyl)(methyl)amino)-3-(5- | |
| | chloro-2-morpholinophenyl)propanoic acid | |
| Int. 25 | (S)-2-((tert-butoxycarbonyl)(methyl)amino)-3-(5- | |
| | chloro-2-morpholinopyridin-3-yl)propanoic acid | |
| Int. 26 | (S)-2-((tert-butoxycarbonyl)(methyl)amino)-3-(7- | |
| | chloro-2,3-dihydrobenzo[b][1,4]dioxin-5- | |
| | yl)propanoic acid | |
| Int. 27 | (S)-2-((tert-butoxycarbonyl)(methyl)amino)-3-(5- | |
| | chloro-2-methylpyridin-3-yl)propanoic acid | |
| Int. 28 | (S)-2-((tert-butoxycarbonyl)(methyl)amino)-3-(5- | |
| | chloro-2-((1-methyl-1H-pyrazol-4-yl)oxy)pyridin- | |
| | 3-yl)propanoic acid | |
| Int. 29 | (S)-3-(2-((1-acetylazetidin-3-yl)oxy)-5- | |
| | chloropyridin-3-yl)-2-((tert- | |
| | butoxycarbonyl)(methyl)amino)propanoic acid | |
| Int. 30 | (S)-3-(2-bromo-5-chlorophenyl)-2-((tert- | |
| | butoxycarbonyl)(methyl)amino)propanoic acid | |
| Int. 31 | (S)-3-(2-bromo-5-fluorophenyl)-2-((tert- | |
| | butoxycarbonyl)(methyl)amino)propanoic acid | |
| Int. 32 | (tert-butoxycarbonyl)-L-alanine | 15761-38-3 |
| Int. 33 | ((2S,4R)-4-fluoro-1-(1- | |
| | (trifluoromethyl)cyclopropane-1- | |
| | carbonyl)pyrrolidine-2-carbonyl)glycine | |
| Int. 34 | ((S)-2-(1-(trifluoromethyl)cyclopropane-1- | |
| | carboxamido)butanoyl)-L-alanine | |
| Int. 35 | (S)-2-((2S,4R)-4-fluoro-1-(1- | |
| | (trifluoromethyl)cyclopropane-1- | |
| | carbonyl)pyrrolidine-2-carboxamido)butanoic acid | |
| Int. 36 | ethyl carbonochloridate | 541-41-3 |
| Int. 37 | isobutyryl chloride | 79-30-1 |
| Int. 38 | pivaloyl chloride | 3282-30-2 |
| Int. 39 | (2S,4R)-4-fluoro-1-(2-(trifluoromethyl)tetrahydro- | |
| | 2H-pyran-2-carbonyl)pyrrolidine-2-carboxylic | |
| | acid | |
| Int. 40 | 1-(4-((4,4,5,5-tetramethyl-1,3,2-dioxaborolan-2- | |
| | yl)methylene)piperidin-1-yl)ethan-1-one | |
| Int. 41 | (S)-2-((tert-butoxycarbonyl)(ethyl)amino)pent-4- | |
| | enoic acid | |
| Int. 42 | (S)-2-((tert-butoxycarbonyl)(methyl)amino)hex-5- | |
| | enoic acid | |
| Int. 43 | (S)-2-((tert-butoxycarbonyl)(methyl)amino)hept-6- | |
| | enoic acid | |
| Int. 44 | 1-(methylsulfonyl)-3-(4,4,5,5-tetramethyl-1,3,2- | |
| | dioxaborolan-2-yl)-2,5-dihydro-1H-pyrrole | |
| Int. 45 | 1-(3-(4,4,5,5-tetramethyl-1,3,2-dioxaborolan-2- | |
| | yl)-8-azabicyclo[3.2.1]oct-2-en-8-yl)ethan-1-one | |
| Int. 46 | 2-(3,6-dihydro-2H-pyran-4-yl)-4,4,5,5- | 287944-16-5 |
| | tetramethyl-1,3,2-dioxaborolane | |
| Int. 47 | 1-(4-(4,4,5,5-tetramethyl-1,3,2-dioxaborolan-2- | 1227068-67-8 |
| | yl)-3,6-dihydropyridin-1(2H)-yl)ethan-1-one | |
| Int. 48 | (3-chlorophenyl)boronic acid | 63503-60-6 |
| Int. 49 | 2-(4,4-difluorocyclohex-1-en-1-yl)-4,4,5,5- | 1227068-84-9 |
| | tetramethyl-1,3,2-dioxaborolane | |
| Int. 50 | 1-(methylsulfonyl)-4-(4,4,5,5-tetramethyl-1,3,2- | 1382137-67-8 |
| | dioxaborolan-2-yl)-1,2,3,6-tetrahydropyridine | |
| Int. 51 | 4-(4,4,5,5-tetramethyl-1,3,2-dioxaborolan-2- | 1310384-24-7 |
| | yl)cyclohex-3-en-1-ol | |
| Int. 52 | 1-methyl-4-(4,4,5,5-tetramethyl-1,3,2- | 761446-44-0 |
| | dioxaborolan-2-yl)-1H-pyrazole | |
| Int. 53 | 1-(oxetan-3-yl)-4-(4,4,5,5-tetramethyl-1,3,2- | 1339890-99-1 |
| | dioxaborolan-2-yl)-1H-pyrazole | |
| Int. 54 | 2-(4-(4,4,5,5-tetramethyl-1,3,2-dioxaborolan-2- | 1040377-08-9 |
| | yl)-1H-pyrazol-1-yl)ethan-1-ol | |
| Int. 55 | pyrimidin-5-ylboronic acid | 109299-78-7 |
| Int. 56 | 1-(2-methoxyethyl)-4-(4,4,5,5-tetramethyl-1,3,2- | 847818-71-7 |
| | dioxaborolan-2-yl)-1H-pyrazole | |
| Int. 57 | 2-(cyclopent-1-en-1-yl)-4,4,5,5-tetramethyl-1,3,2- | 287944-10-9 |
| | dioxaborolane | |
| Int. 58 | 1-methyl-4-(4,4,5,5-tetramethyl-1,3,2- | 454482-11-2 |
| | dioxaborolan-2-yl)-1,2,3,6-tetrahydropyridine | |
| Int. 59 | 1-cyclopropyl-4-(4,4,5,5-tetramethyl-1,3,2- | 1151802-22-0 |
| | dioxaborolan-2-yl)-1H-pyrazole | |
| Int. 60 | 1-(methoxymethyl)-4-(4,4,5,5-tetramethyl-1,3,2- | 1301198-65-1 |
| | dioxaborolan-2-yl)-1H-pyrazole | |
| Int. 61 | 1-(2,2-difluoroethyl)-4-(4,4,5,5-tetramethyl-1,3,2- | 1048730-40-6 |
| | dioxaborolan-2-yl)-1H-pyrazole | |
| Int. 62 | 1-(tetrahydro-2H-pyran-4-yl)-4-(4,4,5,5- | 1040377-03-4 |
| | tetramethyl-1,3,2-dioxaborolan-2-yl)-1H-pyrazole | |
| Int. 63 | 1-(tetrahydrofuran-3-yl)-4-(4,4,5,5-tetramethyl- | 1029715-63-6 |
| | 1,3,2-dioxaborolan-2-yl)-1H-pyrazole | |
| Int. 64 | 1-(methylsulfonyl)-4-(4,4,5,5-tetramethyl-1,3,2- | 944994-03-0 |
| | dioxaborolan-2-yl)-1H-pyrazole | |
| Int. 65 | 1-cyclobutyl-4-(4,4,5,5-tetramethyl-1,3,2- | 1002309-48-9 |
| | dioxaborolan-2-yl)-1H-pyrazole | |
| Int. 66 | cyclopropyl(4-(4,4,5,5-tetramethyl-1,3,2- | 1616388-38-5 |
| | dioxaborolan-2-yl)-3,6-dihydropyridin-1(2H)- | |
| | yl)methanone | |
| Int. 67 | 4-(4,4,5,5-tetramethyl-1,3,2-dioxaborolan-2-yl)- | 1121057-75-7 |
| | 1,2,3,6-tetrahydropyridine hydrochloride | |
| Int. 68 | 4,4,5,5-tetramethyl-2-(prop-1-en-2-yl)-1,3,2- | 126726-62-3 |
| | dioxaborolane | |
| Int. 69 | 4,4,5,5-tetramethyl-2-(2-methylprop-1-en-1-yl)- | 126689-00-7 |
| | 1,3,2-dioxaborolane | |
| Int. 70 | 2-methyl-5-(4,4,5,5-tetramethyl-1,3,2- | 1218791-01-5 |
| | dioxaborolan-2-yl)thiazole | |
| Int. 71 | 1-isopropyl-4-(4,4,5,5-tetramethyl-1,3,2- | 879487-10-2 |
| | dioxaborolan-2-yl)-1H-pyrazole | |
| Int. 72 | cyclopropylboronic acid | 411235-57-9 |
| Int. 73 | 3-(4,4,5,5-tetramethyl-1,3,2-dioxaborolan-2-yl)- | 1314138-13-0 |
| | 5,6-dihydro-4H-pyrrolo[1,2-b]pyrazole | |
| Int. 74 | 1-methyl-4-(4,4,5,5-tetramethyl-1,3,2- | 1083180-01-1 |
| | dioxaborolan-2-yl)-1H-imidazole | |
| Int. 75 | 4-(4,4,5,5-tetramethyl-1,3,2-dioxaborolan-2-yl)- | 269410-08-4 |
| | 1H-pyrazole | |
| Int. 76 | benzo[c][1,2,5]oxadiazol-5-ylboronic acid | 426268-09-9 |
| Int. 77 | 6-(4,4,5,5-tetramethyl-1,3,2-dioxaborolan-2-yl)- | 1218790-54-5 |
| | [1,2,5]oxadiazolo[3,4-b]pyridine | |
| Int. 78 | 6-(4,4,5,5-tetramethyl-1,3,2-dioxaborolan-2- | 1416437-27-8 |
| | yl)pyrazolo[1,5-a]pyrimidine | |
| Int. 79 | 3-methyl-6-(4,4,5,5-tetramethyl-1,3,2- | 1257554-02-1 |
| | dioxaborolan-2-yl)-3H-imidazo[4,5-b]pyridine | |
| Int. 80 | 6-(4,4,5,5-tetramethyl-1,3,2-dioxaborolan-2-yl)- | 1314137-24-0 |
| | [1,2,4]triazolo[4,3-a]pyridine | |
| Int. 81 | 7-(4,4,5,5-tetramethyl-1,3,2-dioxaborolan-2-yl)- | 1210048-18-2 |
| | [1,2,4]triazolo[1,5-a]pyridine | |
| Int. 82 | (6-(ethylthio)pyridin-3-yl)boronic acid | 1217501-38-6 |
| Int. 83 | (6-ethoxypyridin-3-yl)boronic acid | 612845-44-0 |
| Int. 84 | 1-(tert-butyl)-4-(4,4,5,5-tetramethyl-1,3,2- | 1256359-15-5 |
| | dioxaborolan-2-yl)-1H-pyrazole | |
| Int. 85 | 3-(4,4,5,5-tetramethyl-1,3,2-dioxaborolan-2-yl)- | 1383675-83-9 |
| | 6,7-dihydro-5H-pyrazolo[5,1-b][1,3]oxazine | |
| Int. 86 | pyridin-4-ylboronic acid hydrate | 1692-15-5 |
| Int. 87 | (2-methoxypyridin-4-yl)boronic acid | 762262-09-9 |
| Int. 88 | (5-fluoropyridin-3-yl)boronic acid | 872041-86-6 |
| Int. 89 | 5-(4,4,5,5-tetramethyl-1,3,2-dioxaborolan-2-yl)- | 1207623-97-9 |
| | 1,3-dihydro-2H-pyrrolo[2,3-b]pyridin-2-one | |
| Int. 90 | 1-(difluoromethyl)-4-(4,4,5,5-tetramethyl-1,3,2- | 1206640-82-5 |
| | dioxaborolan-2-yl)-1H-pyrazole | |
| Int. 91 | (6-(methylsulfonyl)pyridin-3-yl)boronic acid | 1088496-41-6 |
| Int. 92 | (5-(methylthio)pyridin-3-yl)boronic acid | 477251-98-2 |
| Int. 93 | 1-methyl-3-(4,4,5,5-tetramethyl-1,3,2- | 1020174-04-2 |
| | dioxaborolan-2-yl)-1H-pyrazole | |
| Int. 94 | 2-methyl-5-(4,4,5,5-tetramethyl-1,3,2- | 1192056-62-4 |
| | dioxaborolan-2-yl)oxazole | |
| Int. 95 | 2,4-dimethyl-5-(4,4,5,5-tetramethyl-1,3,2- | 859833-13-9 |
| | dioxaborolan-2-yl)thiazole | |
| Int. 96 | (2-oxo-1,2-dihydropyridin-3-yl)boronic acid | 951655-49-5 |
| Int. 97 | 2-(2,5-dihydrofuran-3-yl)-4,4,5,5-tetramethyl- | 212127-80-5 |
| | 1,3,2-dioxaborolane | |
| Int. 98 | (3,5-dimethylisoxazol-4-yl)boronic acid | 16114-47-9 |
| Int. 99 | 3-methyl-4-(4,4,5,5-tetramethyl-1,3,2- | 936250-20-3 |
| | dioxaborolan-2-yl)-1H-pyrazole | |
| Int. 100 | 5-methyl-4-(4,4,5,5-tetramethyl-1,3,2- | 1346808-41-0 |
| | dioxaborolan-2-yl)isoxazole | |
| Int. 101 | 2-(4-(4,4,5,5-tetramethyl-1,3,2-dioxaborolan-2- | 1093307-35-7 |
| | yl)-1H-pyrazol-1-yl)acetonitrile | |
| Int. 102 | 4-fluoro-1-isopropyl-2-methyl-6-(4,4,5,5- | 1231930-37-2 |
| | tetramethyl-1,3,2-dioxaborolan-2-yl)-1H- | |
| | benzo[d]imidazole | |
| Int. 103 | 6-(4,4,5,5-tetramethyl-1,3,2-dioxaborolan-2-yl)- | 1160790-18-0 |
| | [1,2,4]triazolo[1,5-a]pyridine | |
| Int. 104 | 1,5-dimethyl-4-(4,4,5,5-tetramethyl-1,3,2- | 1036991-40-8 |
| | dioxaborolan-2-yl)-1H-pyrazole | |
| Int. 105 | 1,3-dimethyl-5-(4,4,5,5-tetramethyl-1,3,2- | 847818-79-5 |
| | dioxaborolan-2-yl)-1H-pyrazole | |
| Int. 106 | 7-(4,4,5,5-tetramethyl-1,3,2-dioxaborolan-2- | 908268-52-0 |
| | yl)imidazo[1,2-a]pyridine | |
| Int. 107 | 1-methyl-4-(4,4,5,5-tetramethyl-1,3,2- | 1227068-65-6 |
| | dioxaborolan-2-yl)-5,6-dihydropyridin-2(1H)-one | |
| Int. 108 | 1-(methyl-d3)-4-(4,4,5,5-tetramethyl-1,3,2- | 1022151-50-3 |
| | dioxaborolan-2-yl)-1H-pyrazole | |
| Int. 109 | (2-methyl-2H-pyrazolo[3,4-b]pyridin-5-yl)boronic | 2304634-03-3 |
| | acid | |
| Int. 110 | 1-methyl-5-(4,4,5,5-tetramethyl-1,3,2- | 1312312-78-9 |
| | dioxaborolan-2-yl)-1H-pyrazolo[3,4-b]pyridine | |
| Int. 111 | 1-methyl-5-(4,4,5,5-tetramethyl-1,3,2- | 1107627-02-0 |
| | dioxaborolan-2-yl)-1H-benzo[d]imidazole | |
| Int. 112 | 1-(cyclopropylsulfonyl)-4-(4,4,5,5-tetramethyl- | 1612172-62-9 |
| | 1,3,2-dioxaborolan-2-yl)-1H-pyrazole | |
| Int. 113 | 3-(4-(4,4,5,5-tetramethyl-1,3,2-dioxaborolan-2- | 1233526-31-2 |
| | yl)-1H-pyrazol-1-yl)tetrahydrothiophene 1,1- | |
| | dioxide | |
| Int. 114 | 3-(4-(4,4,5,5-tetramethyl-1,3,2-dioxaborolan-2- | 1776065-66-7 |
| | yl)-1H-pyrazol-1-yl)thietane 1,1-dioxide | |
| Int. 115 | 4-(2-(4-(4,4,5,5-tetramethyl-1,3,2-dioxaborolan-2- | 864754-18-7 |
| | yl)-1H-pyrazol-1-yl)ethyl)morpholine | |
| Int. 116 | 1-(2-(methylsulfonyl)ethyl)-4-(4,4,5,5- | 1339892-52-2 |
| | tetramethyl-1,3,2-dioxaborolan-2-yl)-1H-pyrazole | |
| Int. 117 | 4-(4-(4,4,5,5-tetramethyl-1,3,2-dioxaborolan-2- | 568577-88-8 |
| | yl)phenyl)morpholine | |
| Int. 118 | (4-(methylsulfonyl)phenyl)boronic acid | 149104-88-1 |
| Int. 119 | (6-acetamidopyridin-3-yl)boronic acid | 947533-21-3 |
| Int. 120 | (4-(pyrrolidin-1-ylsulfonyl)phenyl)boronic acid | 486422-57-5 |
| Int. 121 | pyridin-3-ylboronic acid | 1692-25-7 |
| Int. 122 | 1-(3,3-dimethyl-4-(4,4,5,5-tetramethyl-1,3,2- | |
| | dioxaborolan-2-yl)-3,6-dihydropyridin-1(2H)- | |
| | yl)ethan-1-one | |
| Int. 123 | 4-(4,4,5,5-tetramethyl-1,3,2-dioxaborolan-2- | 863422-41-7 |
| | yl)pyridazine | |
| Int. 124 | 3-methyl-5-(4,4,5,5-tetramethyl-1,3,2- | 1350543-95-1 |
| | dioxaborolan-2-yl)pyridazine | |
| Int. 125 | 3-(4,4,5,5-tetramethyl-1,3,2-dioxaborolan-2- | 1169690-88-3 |
| | yl)pyrazolo[1,5-a]pyrimidine | |
| Int. 126 | pyrazolo[1,5-a]pyrazin-3-ylboronic acid | 2304634-86-2 |
| Int. 127 | (S)-2-cyclopropyl-2-((2S,4R)-1-(3,3-difluoro-1- | |
| | (trifluoromethyl)cyclobutane-1-carbonyl)-4- | |
| | fluoropyrrolidine-2-carboxamido)acetic acid | |
| Int. 128 | tert-butyl 3-(4,4,5,5-tetramethyl-1,3,2- | 212127-83-8 |
| | dioxaborolan-2-yl)-2,5-dihydro-1H-pyrrole-1- | |
| | carboxylate | |
| Int. 129 | (S)-2-cyclopropyl-2-((2S,4R)-4-fluoro-1-(1- | |
| | (trifluoromethyl)cyclopropane-1- | |
| | carbonyl)pyrrolidine-2-carboxamido)acetic acid | |
| Int. 130 | Methylboronic acid | 13061-96-6 |
| Int. 131 | 5-(4,4,5,5-Tetramethyl-1,3,2-dioxaborolan-2- | 1073354-91-2 |
| | yl)benzo[d]thiazole | |
| Int. 132 | 5-(4,4,5,5-Tetramethyl-1,3,2-dioxaborolan-2- | 1054483-78-1 |
| | yl)pyridin-2(1H)-one | |
| Int. 133 | 3-(4,4,5,5-tetramethyl-1,3,2-dioxaborolan-2- | 329214-79-1 |
| | yl)pyridine | |
| Int. 134 | tert-butyl 3-(4,4,5,5-tetramethyl-1,3,2- | 900503-08-4 |
| | dioxaborolan-2-yl)-8-azabicyclo[3.2.1]oct-2-ene- | |
| | 8-carboxylate | |
| Int. 135 | tert-butyl 7-(4,4,5,5-tetramethyl-1,3,2- | 1313034-29-5 |
| | dioxaborolan-2-yl)-3-oxa-9-azabicyclo[3.3.1]non- | |
| | 6-ene-9-carboxylate | |
| Int. 137 | tert-butyl (4-(4,4,5,5-tetramethyl-1,3,2- | 1251732-64-5 |
| | dioxaborolan-2-yl)cyclohex-3-en-1-yl)carbamate | |
| Int. 138 | tert-butyl 4-(4,4,5,5-tetramethyl-1,3,2- | 286961-14-6 |
| | dioxaborolan-2-yl)-3,6-dihydropyridine-1(2H)- | |
| | carboxylate | |
| Int. 140 | (2S,4R)-4-fluoro-1-(1- | |
| | (trifluoromethyl)cyclopropane-1- | |
| | carbonyl)pyrrolidine-2-carboxylic acid | |
| Int. 141 | (2S,4R)-1-(3,3-difluoro-1- | |
| | (trifluoromethyl)cyclobutane-1-carbonyl)-4- | |
| | fluoropyrrolidine-2-carboxylic acid | |
| Int. 142 | (2S,4R)-4-fluoro-1-((R)-3,3,3-trifluoro-2-hydroxy- | |
| | 2-methylpropanoyl)pyrrolidine-2-carboxylic acid | |
| Int. 143 | (2S,4R)-4-fluoro-1-(2- | |
| | (trifluoromethyl)spiro[3.3]heptane-2- | |
| | carbonyl)pyrrolidine-2-carboxylic acid | |
| Int. 144 | (2S,4R)-4-fluoro-1-(1- | |
| | (trifluoromethyl)cyclobutane-1- | |
| | carbonyl)pyrrolidine-2-carboxylic acid | |
| Int. 145 | (1-(trifluoromethyl)cyclobutane-1-carbonyl)-L- | |
| | proline | |
| Int. 146 | (3,3-difluoro-1-(trifluoromethyl)cyclobutane-1- | |
| | carbonyl)-L-proline | |
| Int. 147 | (2S,4R)-1-(1-cyanocyclobutane-1-carbonyl)-4- | |
| | fluoropyrrolidine-2-carboxylic acid | |
| Int. 148 | (2S,4R)-4-fluoro-1-((R)-2- | |
| | (trifluoromethyl)tetrahydro-2H-pyran-2- | |
| | carbonyl)pyrrolidine-2-carboxylic acid | |
| Int. 149 | (2S,4R)-4-fluoro-1-((S)-2- | |
| | (trifluoromethyl)tetrahydro-2H-pyran-2- | |
| | carbonyl)pyrrolidine-2-carboxylic acid | |
| Int. 150 | (2S,4R)-4-fluoro-1-(3,3,3-trifluoro-2,2- | |
| | dimethylpropanoyl)pyrrolidine-2-carboxylic acid | |
| Int. 151 | (tert-butoxycarbonyl)-L-leucine | 13139-15-6 |
| Int. 152 | (S)-2-((tert-butoxycarbonyl)(methyl)amino)pent-4- | 136092-76-7 |
| | enoic acid | |
| Int. 153 | (S)-2-((tert-butoxycarbonyl)amino)-2- | 155976-13-9 |
| | cyclopropylacetic acid | |
| Int. 154 | tert-butyl (S)-2-((((9H-fluoren-9- | |
| | yl)methoxy)carbonyl)(methyl)amino)pent-4- | |
| | enoate | |
| Int. 155 | 4-(4-(4,4,5,5-tetramethyl-1,3,2-dioxaborolan-2- | 364794-79-6 |
| | yl)benzyl)morpholine | |
| Int. 156 | 1-methyl-4-(4-(4,4,5,5-tetramethyl-1,3,2- | 938043-30-2 |
| | dioxaborolan-2-yl)benzyl)piperazine | |
| Int. 157 | 1-methyl-4-(4-(4,4,5,5-tetramethyl-1,3,2- | 747413-21-4 |
| | dioxaborolan-2-yl)phenyl)piperazine | |
| Int. 158 | 3-(4-(4,4,5,5-tetramethyl-1,3,2-dioxaborolan-2- | 929203-04-3 |
| | yl)phenyl)pyridine | |
| Int. 159 | 1-methyl-4-(4-(4,4,5,5-tetramethyl-1,3,2- | 747413-21-4 |
| | dioxaborolan-2-yl)phenyl)piperazine | |
| Int. 160 | 4-((4-(4,4,5,5-tetramethyl-1,3,2-dioxaborolan-2- | 1401222-64-7 |
| | yl)phenyl)sulfonyl)morpholine | |
| Int. 161 | 1-methyl-4-(4-(4,4,5,5-tetramethyl-1,3,2- | 1607838-14-1 |
| | dioxaborolan-2-yl)phenyl)-1H-pyrazole | |
| Int. 162 | (S)-1-(3,3-difluoro-1- | |
| | (trifluoromethyl)cyclobutane-1-carbonyl)-4,4- | |
| | difluoropyrrolidine-2-carboxylic acid | |
| Int. 163 | (3-methyl-4-morpholinophenyl)boronic acid | 1426245-63-7 |
| Int. 164 | 4-(2-(4-(4,4,5,5-tetramethyl-1,3,2-dioxaborolan-2- | 690636-28-3 |
| | yl)phenoxy)ethyl)morpholine | |
| Int. 165 | 4-methyl-7-(4,4,5,5-tetramethyl-1,3,2- | 519054-54-7 |
| | dioxaborolan-2-yl)-3,4-dihydro-2H- | |
| | benzo[b][1,4]oxazine | |
| Int. 166 | (3-chloro-4-morpholinophenyl)boronic acid | 1426246-59-4 |
| Int. 167 | (4-((tetrahydro-2H-pyran-4-yl)oxy)phenyl)boronic | 279261-92-6 |
| | acid | |
| Int. 168 | 1-(4-(4,4,5,5-tetramethyl-1,3,2-dioxaborolan-2- | 912369-50-7 |
| | yl)phenyl)piperazine | |
| Int. 169 | 1-isopropyl-4-(4-(4,4,5,5-tetramethyl-1,3,2- | 1073354-18-3 |
| | dioxaborolan-2-yl)phenyl)piperazine | |
| Int. 170 | 1-methyl-4-(4-(4,4,5,5-tetramethyl-1,3,2- | 1247000-92-5 |
| | dioxaborolan-2-yl)phenyl)piperidine | |
| Int. 171 | (3-fluoro-4-morpholinophenyl)boronic acid | 279262-09-8 |
| Int. 172 | 1-methyl-5′-(4,4,5,5-tetramethyl-1,3,2- | |
| | dioxaborolan-2-yl)-3′H-spiro[azetidine-3,1′- | |
| | isobenzofuran] | |
| Int. 173 | 4-(3-fluoro-4-(4,4,5,5-tetramethyl-1,3,2- | 957198-29-7 |
| | dioxaborolan-2-yl)pyridin-2-yl)morpholine | |
| Int. 174 | (2S,4R)-1-(1-(difluoromethyl)-3,3- | |
| | difluorocyclobutane-1-carbonyl)-4- | |
| | fluoropyrrolidine-2-carboxylic acid | |
| Int. 175 | 1-methyl-4-(4-(4,4,5,5-tetramethyl-1,3,2- | 124700-48-9 |
| | dioxaborolan-2-yl)phenoxy)piperidine | |
| Int. 176 | 2-((1S,6R)-3-oxabicyclo[4.1.0]heptan-6-yl)-5- | |
| | (4,4,5,5-tetraethyl-1,3,2-dioxaborolan-2- | |
| | yl)pyridine | |
| Int. 177 | 1-methyl-4-(4-(4,4,5,5-tetramethyl-1,3,2- | 1447696-00-5 |
| | dioxaborolan-2-yl)phenyl)-1,2,3,6- | |
| | tetrahydropyridine | |
| Int. 178 | 8-methyl-3-(4-(4,4,5,5-tetramethyl-1,3,2- | |
| | dioxaborolan-2-yl)phenyl)-3,8- | |
| | diazabicyclo[3.2.1]octane | |
| Int. 179 | (1S,4S)-2-methyl-5-(4-(4,4,5,5-tetramethyl-1,3,2- | |
| | dioxaborolan-2-yl)phenyl)-2,5- | |
| | diazabicyclo[2.2.1]heptane | |
| Int. 180 | (2S,4R)-1-(3,3-difluoro-1-methylcyclobutane-1- | |
| | carbonyl)-4-fluoropyrrolidine-2-carboxylic acid | |
| Int. 181 | 2,2-dimethyl-4-(4-(4,4,5,5-tetramethyl-1,3,2- | |
| | dioxaborolan-2-yl)phenyl)morpholine | |
| Int. 182 | 4-(2-methoxy-4-(4,4,5,5-tetramethyl-1,3,2- | |
| | dioxaborolan-2-yl)phenyl)morpholine | |
| Int. 183 | 1-(pyridin-2-ylmethyl)-4-(4-(4,4,5,5-tetramethyl- | |
| | 1,3,2-dioxaborolan-2-yl)phenyl)piperazine | |
| Int. 184 | (S)-3-(methoxymethyl)-4-(4-(4,4,5,5-tetramethyl- | |
| | 1,3,2-dioxaborolan-2-yl)phenyl)morpholine | |
| Int. 185 | 1-methyl-4-(2-methyl-4-(4,4,5,5-tetramethyl- | 1310704-51-8 |
| | 1,3,2-dioxaborolan-2-yl)phenyl)piperazine | |
| Int. 186 | 4-fluoro-1-methyl-4-(4-(4,4,5,5-tetramethyl-1,3,2- | |
| | dioxaborolan-2-yl)phenyl)piperidine | |
| Int. 187 | 2-morpholino-5-(4,4,5,5-tetramethyl-1,3,2- | |
| | dioxaborolan-2-yl)benzonitrile | |
| Int. 188 | 1-methyl-4-(4-(4,4,5,5-tetramethyl-1,3,2- | 1046793-62-7 |
| | dioxaborolan-2-yl)cyclohex-3-en-1-yl)piperazine | |
| Int. 189 | (2S,4R)-4-fluoro-1-(2,3,3,3-tetrafluoro-2- | |
| | methylpropanoyl)pyrrolidine-2-carboxylic acid | |
| Int. 190 | 3-(4-(4,4,5,5-tetramethyl-1,3,2-dioxaborolan-2- | |
| | yl)phenyl)-6,7-dihydro-4H-pyrazolo[5,1- | |
| | c][1,4]oxazine . . . | |
| Int. 191 | 1-methyl-4-(4-(4-(4,4,5,5-tetramethyl-1,3,2- | |
| | dioxaborolan-2-yl)phenyl)-1H-pyrazol-1- | |
| | yl)piperidine . . . | |
| Int. 192 | (2R,6R)-2,6-dimethyl-4-(4-(4,4,5,5-tetramethyl- | |
| | 1,3,2-dioxaborolan-2-yl)phenyl)morpholine | |
| Int. 193 | tert-butyl 3-(4-(4-(4,4,5,5-tetramethyl-1,3,2- | |
| | dioxaborolan-2-yl)phenyl)piperazin-1- | |
| | yl)azetidine-1-carboxylate | |
| Int. 194 | (4-(1-(4-Methylpiperazin-1- | 1643923-77-6 |
| | yl)cyclopropyl)phenyl)bornoic acid pinacol ester | |
| Int. 195 | N-ethylhex-5-en-1-amine | |
| Int. 196 | (2R,6S)-1,2,6-trimethyl-4-(4-(4,4,5,5-tetramethyl- | |
| | 1,3,2-dioxaborolan-2-yl)phenyl)piperazine . . . | |
| Int. 197 | 3-[4-(4,4,5,5-Tetramethyl-[1,3,2]dioxaborolan-2- | 1362703-30-7 |
| | yl)-phenoxy]-pyridine | |
| Int. 198 | 2,3-Difluoro-4-morpholinophenylboronic acid | 2377609-97-5 |
| Int. 199 | 1-(oxetan-3-yl)-4-(4-(4,4,5,5-tetramethyl-1,3,2- | |
| | dioxaborolan-2-yl)phenyl)piperazine | |
| Int. 400 | (3aR,6aS)-5-(4-(4,4,5,5-tetramethyl-1,3,2- | |
| | dioxaborolan-2-yl)phenyl)hexahydro-1H-furo[3,4- | |
| | c]pyrrole | |
| Int. 401 | (R)-8-(4-(4,4,5,5-tetramethyl-1,3,2-dioxaborolan- | |
| | 2-yl)phenyl)octahydropyrazino[2,1-c][1,4]oxazine | |
| Int. 402 | 4-(3-methoxy-4-(4,4,5,5-tetramethyl-1,3,2- | 1366131-51-2 |
| | dioxaborolan-2-yl)phenyl)morpholine | |
| Int. 403 | 1-(2-(2-methoxyethoxy)ethyl)-4-(4-(4,4,5,5- | |
| | tetramethyl-1,3,2-dioxaborolan-2- | |
| | yl)phenyl)piperazine | |
| Int. 404 | 1-(2,2-difluoroethyl)-4-(4-(4,4,5,5-tetramethyl- | |
| | 1,3,2-dioxaborolan-2-yl)phenyl)piperazine | |
| Int. 405 | 1-((1-methyl-1H-pyrazol-4-yl)methyl)-4-(4- | |
| | (4,4,5,5-tetramethyl-1,3,2-dioxaborolan-2- | |
| | yl)phenyl)piperazine | |
| Int. 406 | (S)-2-((tert-butoxycarbonyl)(methyl)amino)-4- | |
| | methoxy-4-oxobutanoic acid | |
| Int. 407 | (S)-2-((tert-butoxycarbonyl)amino)-5-methoxy-5- | 45214-91-3 |
| | oxopentanoic acid | |
| Int. 408 | (S)-2-((tert-butoxycarbonyl)(methyl- | |
| | d3)amino)pent-4-enoic acid | |
| Int. 409 | [4-(4-Methylpiperazine-1-carbonyl)phenyl]boronic | 832114-06-4 |
| | acid pinacol ester | |
| Int. 410 | 1-methyl-4-(5-(4,4,5,5-tetramethyl-1,3,2- | 918524-63-7 |
| | dioxaborolan-2-yl)pyridin-2-yl)piperazine | |
| Int. 411 | 7-bromo-5-chloro-1-methyl-1H-indole | |
| Int. 412 | 4-bromo-6-chloro-1-methyl-1H-indole | |
| Int. 413 | 7-bromo-5-chlorobenzofuran | 286836-07-5 |
| Int. 414 | 2-((1-methylpiperidin-4-yl)oxy)-5-(4,4,5,5- | |
| | tetramethyl-1,3,2-dioxaborolan-2-yl)pyridine | |
| Int. 415 | (2S,4R)-1-(1-chlorocyclopropane-1-carbonyl)-4- | |
| | fluoropyrrolidine-2-carboxylic acid | |
| Int. 416 | (2S,4R)-1-((R)-2,2-difluoro-1- | |
| | methylcyclopropane-1-carbonyl)-4- | |
| | fluoropyrrolidine-2-carboxylic acid | |
| Int. 417 | (2S,4R)-1-((S)-2,2-difluoro-1- | |
| | methylcyclopropane-1-carbonyl)-4- | |
| | fluoropyrrolidine-2-carboxylic acid . . . | |
| Int. 418 | 1-(2-fluoro-4-(4,4,5,5-tetramethyl-1,3,2- | 1408088-34-5 |
| | dioxaborolan-2-yl)phenyl)-4-methylpiperazine | |
| Int. 419 | 2-(3-fluoro-3-methylazetidin-1-yl)-5-(4,4,5,5- | |
| | tetramethyl-1,3,2-dioxaborolan-2-yl)pyridine | |
| Int. 420 | 4-(1-(4-(4,4,5,5-Tetramethyl-1,3,2-dioxaborolan- | 1206594-12-8 |
| | 2-yl)phenyl)ethyl)morpholine | |
| Int. 421 | 2-methyl-7-(4,4,5,5-tetramethyl-1,3,2- | |
| | dioxaborolan-2-yl)-1,2,3,4-tetrahydroisoquinoline | |
| Int. 422 | 2-Methyl-1,2,3,4-tetrahydroisoquinoline-6- | 922718-57-8 |
| | boronicacid pinacol ester | |
| Int. 423 | 4-[4-(4,4,5,5-tetramethyl-1,3,2-dioxaborolan-2- | 928631-04-3 |
| | yl)phenyl]morpholin-3- one | |
| Int. 424 | 2-methyl-6-(4-(4,4,5,5-tetramethyl-1,3,2- | |
| | dioxaborolan-2-yl)phenyl)-2,6- | |
| | diazaspiro[3.4]octane . . . | |
| Int. 425 | (S)-1,2-dimethyl-4-(4-(4,4,5,5-tetramethyl-1,3,2- | |
| | dioxaborolan-2-yl)phenyl)piperazine . . . | |
| Int. 426 | (S)-N,N-dimethyl-1-(4-(4-(4,4,5,5-tetramethyl- | |
| | 1,3,2-dioxaborolan-2-yl)phenyl)morpholin-2- | |
| | yl)methanamine | |
| Int. 427 | (R)-1-methyl-3-(4-(4,4,5,5-tetramethyl-1,3,2- | |
| | dioxaborolan-2-yl)phenyl)piperidine . . . | |
| Int. 428 | (S)-1-methyl-3-(4-(4,4,5,5-tetramethyl-1,3,2- | |
| | dioxaborolan-2-yl)phenyl)piperidine | |
| Int. 429 | 1-(2-fluoro-4-(4,4,5,5-tetramethyl-1,3,2- | |
| | dioxaborolan-2-yl)phenyl)-4-isopropylpiperazine . . . | |
| Int. 430 | (R)-2-methyl-4-(4-(4,4,5,5-tetramethyl-1,3,2- | |
| | dioxaborolan-2-yl)phenyl)morpholine | |
| Int. 431 | (S)-1-methyl-2-(4-(4,4,5,5-tetramethyl-1,3,2- | |
| | dioxaborolan-2-yl)phenyl)piperidine | |
| Int. 432 | 8-Bromo-6-fluoroquinoline | 22960-18-5 |
| Int. 433 | 4-bromo-6-chloro-1-methyl-1H-indazole | |
| Int. 434 | 1-Bromo-3-chloronaphthalene | 51671-06-8 |
| Int. 435 | 2-bromo-4-chlorothiophene | 32431-92-8 |
| Int. 436 | 5-Bromo-2-cyanopyridine | 97483-77-7 |
| Int. 437 | 1-Methylpiperazine | 109-01-3 |
| Int. 438 | (S)-3-(2-bromo-5-chlorophenyl)-2-((tert- | |
| | butoxycarbonyl)amino)propanoic acid | |

### Int. 6: Preparation of (S)-2-((tert-butoxycarbonyl)(methyl)amino)-3-(2,5-dichlorophenyl)propanoic acid

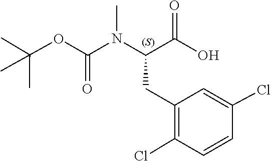

### Step 1: Methyl (2S)-2-[(tert-butoxycarbonyl)amino]-3-(2,5-dichlorophenyl)propanoate

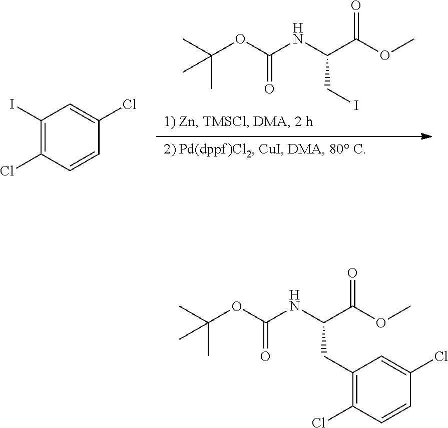

To a stirred mixture of Zn (29.95 g, 458.06 mmol, 2.5 eq) and TMSCl (1.33 g, 12.28 mmol, 0.067 eq) in DMA was added methyl (2R)-2-[(tert-butoxycarbonyl)amino]-3-iodopropanoate (1.03 g, 3.11 mmol, 1.7 eq) dropwise below 45° C. under nitrogen atmosphere. The resulting mixture was stirred for 16 h at rt under nitrogen atmosphere. The above mixture was added into a solution of 1,4-dichloro-2-iodo-benzene (50 g, 183.22 mmol, 1 eq), CuI (6.96 g, 36.64 mmol, 0.2 eq) and Pd(dppf)Cl₂·CH₂Cl₂ (14.93 g, 18.32 mmol, 0.10 eq) at rt under nitrogen. The resulting mixture was stirred for additional 16 h at 85° C. Desired product could be detected by LCMS. The reaction was quenched by the addition of water (1.5 L) at rt. The aqueous layer was extracted with EtOAc (3×500 mL). The combined organics were washed with (5×300 mL) of water and concentrated under reduced pressure. The residue was purified by silica gel column chromatography, eluted with PE/EA (25:1) to afford methyl (2S)-2-[(tert-butoxycarbonyl)amino]-3-(2,5-dichlorophenyl)propanoate (40 g, 63%) as a white solid. LCMS (ESI+): m/z 292.15 (M−56⁺). ¹H NMR (300 MHz, DMSO-d₆): δ 1.11-1.47 (m, 12H), 2.89 (dd, 1H), 3.22 (dd, 1H), 3.64 (d, 4H), 3.91-4.37 (m, 1H), 6.87-7.89 (m, 4H).

### Step 2: Methyl (2S)-2-[(tert-butoxycarbonyl)(methyl)amino]-3-(2,5-dichlorophenyl)propanoate

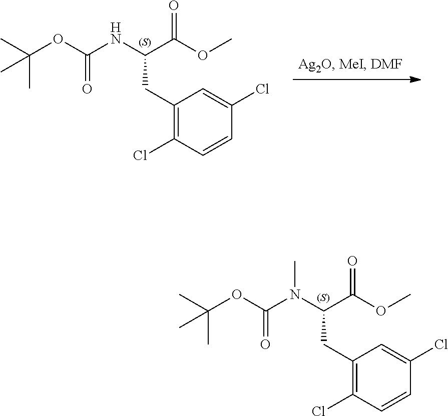

To a stirred solution of methyl (2S)-2-[(tert-butoxycarbonyl)amino]-3-(2,5-dichlorophenyl)propanoate (200 mg, 0.574 mmol, 1 eq) and methyl iodide (214.00 g, 1507.67 mmol, 15 eq) in DMF (350 mL) was added (argentiooxy)silver (93.17 g, 402.04 mmol, 4 eq) in portions at 25° C. under nitrogen atmosphere. The reaction mixture was stirred for 16 h at 25° C. Desired product could be detected by LCMS. The resulting mixture was filtered, the filter cake was washed with EtOAc (3×100 mL). The filtrate was diluted water (500 mL) and extracted with EtOAc (3×300 mL). The combined organic layers were washed with water (4×200 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.05% TFA), 10% to 50% gradient in 10 min; detector, UV 220 nm. This resulted in methyl (2S)-2-[(tert-butoxycarbonyl)(methyl)amino]-3-(2,5-dichlorophenyl)propanoate as a white solid. LCMS (ESI+): m/z 262.20 (M+Na⁺).

### Step 3: Int. 6

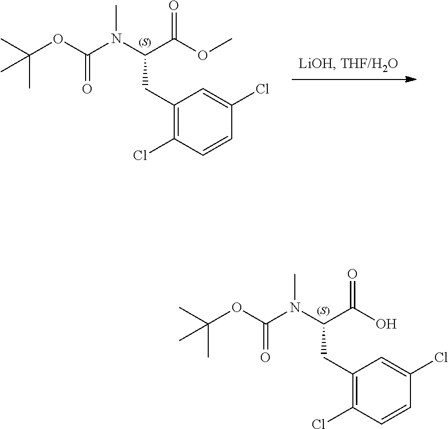

A solution of methyl (2S)-2-[(tert-butoxycarbonyl)(methyl)amino]-3-(2,5-dichlorophenyl)propanoate (35 g, 96.62 mmol, 1 eq) and LiOH (4.63 g, 193.24 mmol, 2 eq) in THF (500 mL)/H₂O (300 mg) was stirred for 16 h at 25° C. Desired product could be detected by LCMS. The mixture was acidified to pH=4 with diluted HCl (aq.). The aqueous layer was extracted with EtOAc (3×500 mL). The combined organics was concentrated under reduced pressure. The residue was purified by reversed-phase flash chromatography with the following conditions: column, C18 silica gel; mobile phase, MeCN in Water (0.1% FA), 10% to 50% gradient in 10 min; detector, UV 220 nm. This resulted in Int. 6 (25.26 g, 75.07%) as a light-yellow solid. LCMS (ESI+): m/z 346.00 (M−H⁻). ¹H NMR (400 MHz, DMSO-d₆): δ 1.23 (d, 9H), 2.66 (d, 3H), 3.06-3.39 (m, 2H), 7.24-7.56 (m, 3H), 13.00 (s, 1H).

### Int. 7: Preparation of (S)-2-((tert-butoxycarbonyl)(methyl)amino)-3-(5-chloro-2-cyclopropoxypyridin-3-yl)propanoic acid

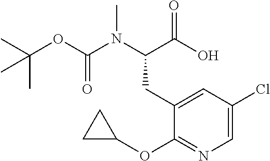 [SMILES: CN(C(=O)OC(C)(C)C)[C@@H](Cc1cc(Cl)cnc1OC1CC1)C(=O)O]

### Step 1: 3-Bromo-5-chloro-2-cyclopropoxypyridine

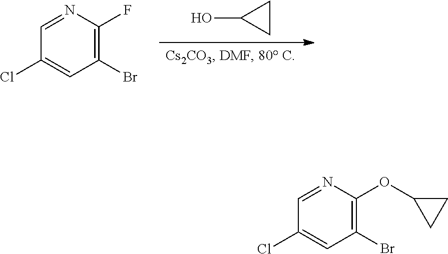

Into a 1000 mL round-bottom flask were added 3-bromo-5-chloro-2-fluoropyridine (50 g, 237.61 mmol, 1 eq) in DMF (400 mL), Cs₂CO₃ (232.97 g, 712.83 mmol, 3 eq) was added at rt under nitrogen atmosphere. The mixture was stirred at rt for 50 min. cyclopropanol (9.94 g, 171.08 mmol, 1.2 eq) was added dropwise over 2 min at rt. The resulting mixture was stirred at 80° C. for 16 h. The reaction was quenched with ice-water (700 mL) and extracted with EA (3×100 mL). The organic layer combined and washed with brine (300 mL), dried over anhydrous Na₂SO₄. After filtration, the filtrate was concentrated under reduced pressure. The residue was purified by silica gel column chromatography, eluted with PE/EA (0˜20%). This resulted in 3-bromo-5-chloro-2-cyclopropoxypyridine (40 g, 68%) as a colorless oil. LCMS (ESI+): m/z 249.95. ¹H NMR (400 MHz, DMSO-d₆) δ 0.76 (ddd, J=5.7, 4.5, 3.1 Hz, 2H), 0.96-0.78 (m, 2H), 4.32 (tt, J=6.4, 3.1 Hz, 1H), 8.17 (d, J=2.5 Hz, 1H), 8.26 (d, J=2.4 Hz, 1H).

### Step 2: Methyl (2S)-2-[(tert-butoxycarbonyl)amino]-3-(5-chloro-2-cyclopropoxypyridin-3-yl)propanoate

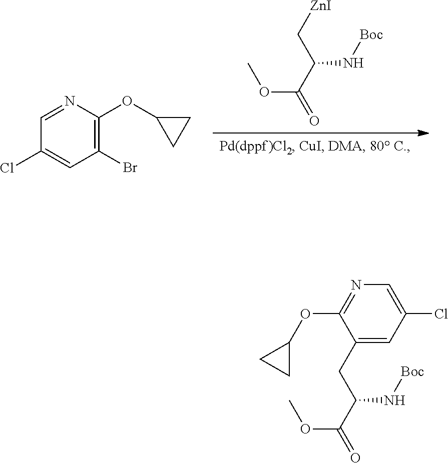

To a mixture of Zn (26.84 g, 410.46 mmol, 1.7 eq) in DMA (135 mL) was added 1,2-dibromoethane (6.80 g, 36.22 mmol, 0.15 eq) in one portion under N₂. Then chlorotrimethylsilane (2.62 g, 24.15 mmol, 0.1 eq) was added slowly and the mixture was stirred for 30 min at 25° C. A solution of methyl 2-[(tert-butoxycarbonyl)amino]-3-iodopropanoate (95.36 g, 289.74 mmol, 1.2 eq) in DMA (135 mL) was added dropwise slowly (60 min) to maintain temperature below 50° C., the resulting mixture was stirred at rt for 2 h and then added 1000 mL 3-necked round-bottom flask a cannula to a solution of 3-bromo-5-chloro-2-cyclopropoxypyridine (60 g, 241.45 mmol, 1 eq), Pd(dppf)Cl₂·CH₂Cl₂ (19.67 g, 24.14 mmol, 0.1 eq) and CuI (9.20 g, 48.29 mmol, 0.2 eq) in DMA (135 ml) under N₂, the color of the mixture turned brown, then the mixture was heated and stirred at 80° C. for 2 h under N₂. The mixture was quenched with ice-water (700 ml) and extracted with EA (3×500 ml). The organic layer was combined and washed with brine (300 ml), dried with anhydrous Na₂SO₄, filtered, and concentrated in vacuum to give the crude product. The crude product was purified by silica gel chromatography eluted with PE/EA (0-50%) to afford methyl (2S)-2-[(tert-butoxycarbonyl)amino]-3-(5-chloro-2-cyclopropoxypyridin-3-yl)propanoate (30 g, 34%) as a white solid. LCMS (ESI+): m/z 371.10.

### Step 3: Methyl (2S)-2-[(tert-butoxycarbonyl)(methyl)amino]-3-(5-chloro-2-cyclopropoxypyridin-3-yl)propanoate

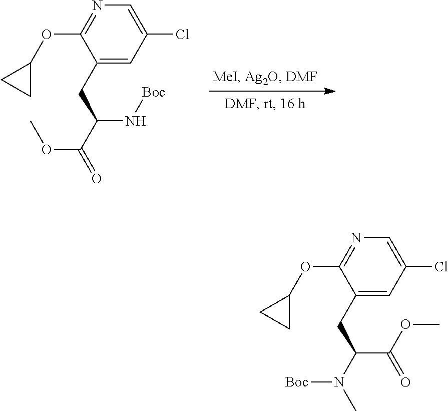

Into a 1000 mL round-bottom flask were added methyl (2S)-2-[(tert-butoxycarbonyl)amino]-3-(5-chloro-2-cyclopropoxypyridin-3-yl)propanoate (25 g, 67.42 mmol, 1 eq) in DMF (400 mL), Ag₂O (78.11 g, 337.08 mmol, 5 eq) was added at 0° C. under nitrogen atmosphere. The mixture was stirred at 0° C. for 30 min, CH₃I (95.69 g, 674.16 mmol, 10 eq) was added dropwise over 2 min at 0° C. The resulting mixture was stirred at rt for 16 h. The reaction was quenched with ice-water (500 mL) and extracted with EtOAc (3×500 mL). The organic layer was combined and washed with brine (300 mL) and dried over anhydrous Na₂SO₄. After filtration, the filtrate was concentrated under reduced pressure. The residue was purified by silica gel column chromatography, eluted with PE/EA (7:1) to afford methyl (2S)-2-[(tert-butoxycarbonyl)(methyl)amino]-3-(5-chloro-2-cyclopropoxypyridin-3-yl)propanoate (22 g, 85%) as a colorless oil. LCMS (ESI+): m/z 385.10. ¹H NMR (400 MHz, DMSO-d₆) δ 0.83-0.62 (m, 2H), 1.19 (t, J=7.1 Hz, 2H), 1.28 (s, 9H), 2.65 (d, J=3.3 Hz, 3H), 2.95 (dd, J=13.6, 10.4 Hz, 1H), 3.07 (ddd, J=14.2, 4.9, 1.9 Hz, 2H), 3.73-3.61 (m, 3H), 4.69 (dd, J=10.5, 4.6 Hz, 1H), 8.12 (dd, J=17.7, 2.6 Hz, 2H).

### Step 4: Int. 7

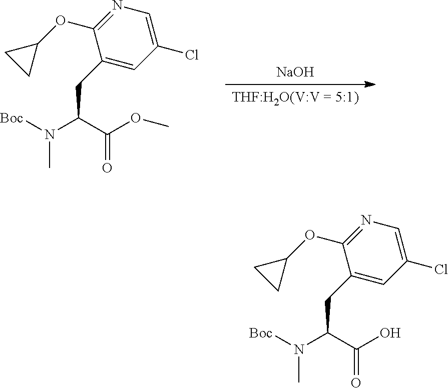

Into a 1000 mL round-bottom flask were added methyl (2S)-2-[(tert-butoxycarbonyl)(methyl)amino]-3-(5-chloro-2-cyclopropoxypyridin-3-yl)propanoate (20 g, 51.97 mmol, 1 eq) in THF (250 ml) and NaOH (10.39 g, 259.84 mmol, 5 eq) in water (50 ml) was added dropwise at 0° C. under nitrogen atmosphere. The resulting mixture was stirred at rt for 2 h. The solvent was removed by reduce pressure and the residue was purified by reverse flash chromatography with the following conditions: column, C18; mobile phase, ACN in water (0.5% FA), 0% to 100% gradient in 40 min; detector, UV 254 nm. This resulted in Int. 7 (13.95 g, 72%) as a yellow oil. LCMS (ESI+): m/z 371.00. ¹H NMR (400 MHz, DMSO-d₆) δ 0.79-0.61 (m, 4H), 1.21 (s, 9H), 2.95-2.84 (m, 2H), 3.00 (s, 3H), 3.09-3.01 (m, 1H), 4.86 (dd, J=11.3, 4.6 Hz, 1H), 7.67 (d, J=2.6 Hz, 1H), 8.11 (dd, J=18.4, 2.6 Hz, 1H).

### Int. 8: Preparation of (S)-2-((tert-butoxycarbonyl)(methyl)amino)-3-(5-chloro-2-(5-methyl-1,3,4-oxadiazol-2-yl)phenyl)propanoic acid

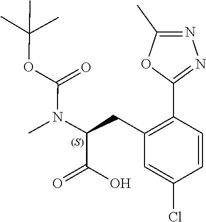

### Step 1: 4-chloro-2-iodobenzohydrazide

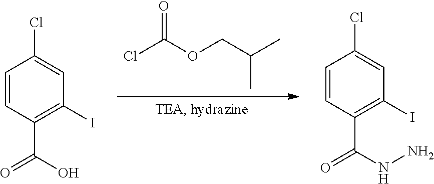

Into a stirred mixture of 4-chloro-2-iodobenzoic acid (10 g, 35.40 mmol, 1 eq) and 2-methylpropyl carbonochloridate (4.84 g, 35.40 mmol, 1 eq) in THF (100 mL) were added TEA (10.75 g, 106.21 mmol, 3 eq) and hydrazine (5.67 g, 177.02 mmol, 5 eq) dropwise at 0° C. under nitrogen atmosphere. The resulting mixture was stirred for an additional 4 h at r. Desired product could be detected by LCMS. The resulting mixture was diluted with water (100 mL). The resulting mixture was extracted with EtOAc (3×100 mL). The combined organic layers were washed with 0.5N HCl solution (3×50 mL). The combined aqueous layers were basified to pH 8 with NaOH (0.5N). The resulting mixture was extracted with EtOAc (3×50 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 5% to 100% gradient in 30 min; detector, UV 254 nm. This resulted in 4-chloro-2-iodobenzohydrazide (7.5 g, 72%) as a white solid. LCMS (ESI+): m/z 296.90 (M+H⁺).

### Step 2: 2-(4-chloro-2-iodophenyl)-5-methyl-1,3,4-oxadiazole

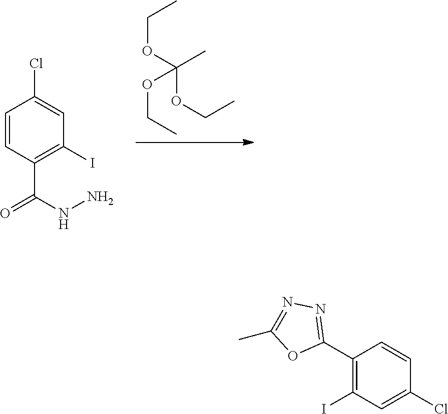

Into a 200 mL vial were added 4-chloro-2-iodobenzohydrazide (8 g, 26.98 mmol, 1 eq) and triethyl orthoacetate (80 mL) at rt. The resulting mixture was stirred overnight at 140° C. Desired product could be detected by LCMS. The residue was purified by reversed-phase flash chromatography with the following conditions: column, C18 silica gel; mobile phase, MeCN in water (0.1% TFA), 5% to 100% gradient in 30 min; detector, UV 254 nm. This resulted in 2-(4-chloro-2-iodophenyl)-5-methyl-1,3,4-oxadiazole (10.2 g, 118%) as a yellow solid. LCMS (ESI+): m/z 320.85 (M+H⁺).

### Step 3: methyl (2S)-2-[(tert-butoxycarbonyl)amino]-3-[5-chloro-2-(5-methyl-1,3,4-oxadiazol-2-yl)phenyl]propanoate

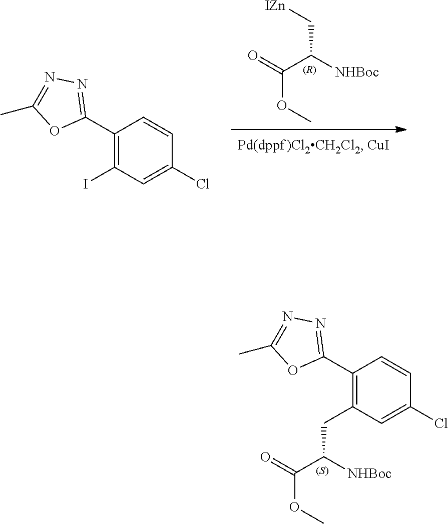

Into a stirred solution of in Zn (3.84 g, 58.73 mmol, 2 eq) DMA (30 mL) were added 1,2-dibromoethane (0.55 g, 2.937 mmol, 0.1 eq) and TMSCl (0.21 g, 1.967 mmol, 0.067 eq) dropwise at rt under nitrogen atmosphere. To the above mixture was added methyl (2R)-2-[(tert-butoxycarbonyl)amino]-3-iodopropanoate (9.66 g, 29.37 mmol, 1 eq) in DMA (30 mL) dropwise. The resulting mixture was stirred for an additional 3 h at rt. To a stirred mixture of 2-(4-chloro-2-iodophenyl)-1,3,4-oxadiazole (9 g, 29.37 mmol, 1 eq) in DMA (30 mL) were added Pd(dppf)Cl₂CH₂Cl₂ (2.39 g, 2.94 mmol, 0.10 eq) and CuI (1.12 g, 5.87 mmol, 0.2 eq) in portions at rt under nitrogen atmosphere. Then, two mixtures were added together. The resulting mixture was stirred for an additional 16 h at rt. The resulting mixture was stirred for additional 3 h at 80° C. Desired product could be detected by LCMS. The reaction was quenched with water 100 mL) at rt. The resulting mixture was filtered, and the filter cake was washed with EtOAc (3×150 mL). The filtrate was concentrated under reduced pressure. The resulting mixture was extracted with EtOAc (3×100 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 5% to 100% gradient in 30 min; detector, UV 254 nm. This resulted in methyl (2S)-2-[(tert-butoxycarbonyl)amino]-3-[5-chloro-2-(5-methyl-1,3,4-oxadiazol-2-yl)phenyl]propanoate (4.5 g, 39%) as a yellow solid. LCMS (ESI+): m/z 396.1 (M+H⁺).

### Step 4: (2S)-2-[(tert-butoxycarbonyl)amino]-3-[5-chloro-2-(5-methyl-1,3,4-oxadiazol-2-yl)phenyl]propanoic acid

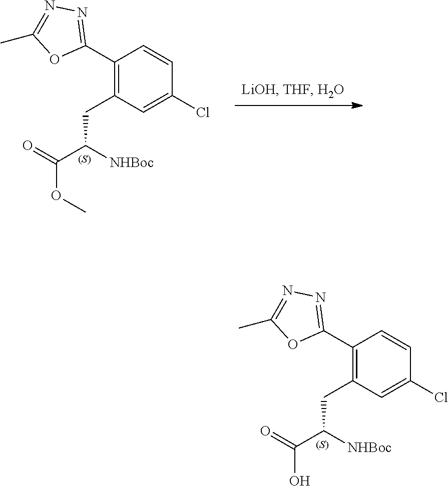

A solution of methyl (2S)-2-[(tert-butoxycarbonyl)amino]-3-[5-chloro-2-(5-methyl-1,3,4-oxadiazol-2-yl)phenyl]propanoate (5 g, 12.63 mmol, 1 eq) in THF (40 mL) and water (10 mL) was treated with LiOH (1.51 g, 63.16 mmol, 5 eq) for 4 h at rt. Desired product could be detected by LCMS. The mixture was diluted with water and EA. The water phase was acidified by diluted HCl (0.5N) and extracted with EA. The organic layer was dried over anhydrous Na2SO4 and concentrated in vacuo. This resulted in (2S)-2-[(tert-butoxycarbonyl) amino]-3-[5-chloro-2-(5-methyl-1,3,4-oxadiazol-2-yl)phenyl]propanoic acid (4.5 g) as a light-yellow solid. LCMS (ESI+): m/z 404.0 (M+Na⁺).

### Step 5: methyl (2S)-3-[5-chloro-2-(5-methyl-1,3,4-oxadiazol-2-yl)phenyl]-2-[(isopropoxycarbonyl)(methyl)amino]propanoate

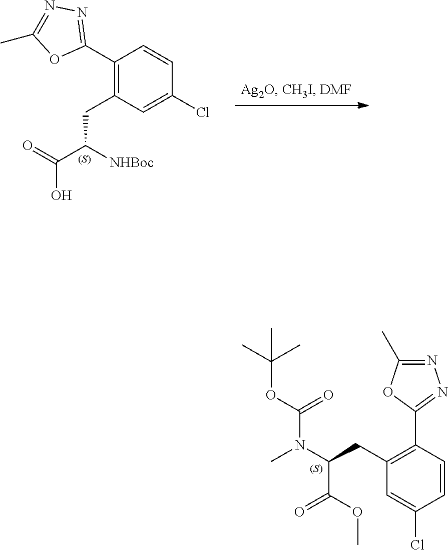

A solution of (2S)-2-[(tert-butoxycarbonyl)amino]-3-[5-chloro-2-(5-methyl-1,3,4-oxadiazol-2-yl)phenyl]propanoic acid (4.5 g, 11.79 mmol, 1 eq) in DMF (50 mL) was treated with Ag₂O (13.66 g, 58.93 mmol, 5 eq) for 20 min at rt under nitrogen atmosphere followed by the addition of CH₃I (25.09 g, 176.79 mmol, 15 eq) dropwise at rt. The resulting mixture was stirred for 4 h at rt. The resulting mixture was diluted with water (50 mL). The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% TFA), 5% to 100% gradient in 50 min; detector, UV 220 nm. This resulted in methyl (2S)-3-[5-chloro-2-(5-methyl-1,3,4-oxadiazol-2-yl)phenyl]-2-[(isopropoxycarbonyl)(methyl)amino]propanoate (3.7 g, 79%) as a yellow solid. LCMS (ESI+): m/z 410.1 (M+H⁺).

### Step 6: Int. 8

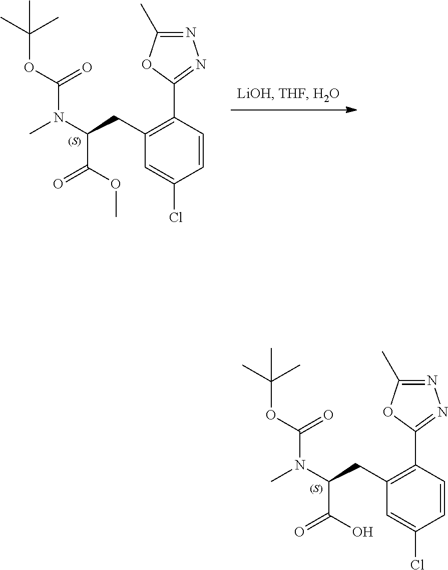

A solution of methyl (2S)-2-[(tert-butoxycarbonyl)(methyl)amino]-3-[5-chloro-2-(5-methyl-1,3,4-oxadiazol-2-yl)phenyl]propanoate (2.6 g, 6.34 mmol, 1 eq) in THF (40 mL) and H₂O (10 mL) was treated with LiOH (1.08 g, 45.14 mmol, 5 eq) for 4 h at rt under nitrogen atmosphere. Desired product could be detected by LCMS. The resulting mixture was diluted with water (20 mL) and acidified to pH 6 with HCl (0.5 mol/L). The mixture was concentrated under reduced pressure and extracted with EtOAc (3×50 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% TFA), 5% to 100% gradient in 30 min; detector, UV 254 nm. This resulted in Int. 8 (3.4289 g, 96%) as a yellow solid. LCMS (ESI+): m/z 390.10 (M+H⁺). ¹H NMR (300 MHz, DMSO-d₆): δ 1.26 (s, 4H), 1.39 (s, 5H) 2.67 (d, J=1.9 Hz, 3H), 2.84 (d, J=8.8 Hz, 3H), 3.33 (ddd, J=14.3, 10.9, 3.4 Hz, 1H), 3.91 (dt, J=14.6, 2.9 Hz, 1H), 5.06 (ddd, J=18.1, 10.8, 3.8 Hz, 1H), 7.34-7.26 (m, 1H), 7.40 (ddd, J=8.5, 6.1, 2.1 Hz, 1H), 7.90 (dd, J=8.4, 3.8 Hz, 1H), 10.00 (s, 1H).

### Int. 9: Preparation of (S)-2-((tert-butoxycarbonyl)(methyl)amino)-3-(2-cyclopropoxy-5-fluoropyridin-3-yl)propanoic acid

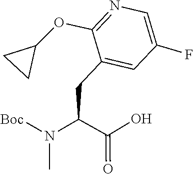 [SMILES: CN(C(=O)OC(C)(C)C)[C@@H](Cc1cc(F)cnc1OC1CC1)C(=O)O]

### Step 1: 3-bromo-2-cyclopropoxy-5-fluoropyridine

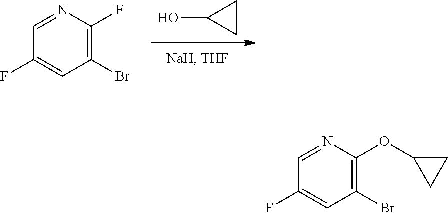

To a solution of cyclopropanol (15.55 g, 268.10 mmol, 1.3 eq) in THF (500 mL) was added sodium hydride (16.6 g, 691.67 mmol, 2 eq) portion wise at 0° C. under nitrogen atmosphere. The mixture was stirred at 0° C. for 1 h. 3-bromo-2,5-difluoropyridine (40 g, 206.21 mmol, 1 eq) was added dropwise over 30 min at 0° C. The resulting mixture was stirred at rt for 16 h. The reaction was quenched with ice-water (700 mL) and extracted with DCM (3×100 mL). The organic layer combined and washed with brine (300 mL), dried over anhydrous Na₂SO₄. After filtration, the filtrate was concentrated under reduced pressure to afford 3-bromo-2-cyclopropoxy-5-fluoropyridine (34 g, crude) as a yellow oil. The crude product was used in the next step directly without further purification. LCMS (ESI+): m/z 233.90 (M+H⁺).

### Step 2: Methyl (2S)-2-[(tert-butoxycarbonyl)amino]-3-(2-cyclopropoxy-5-fluoropyridin-3-yl)propanoate

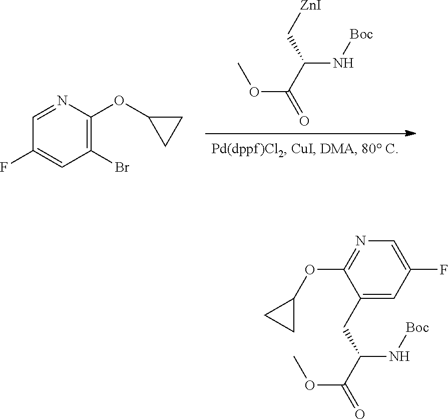

To a mixture of Zn (15.94 g, 249.06 mmol, 1.7 eq) in DMA (80 mL) was added 1,2-dibromoethane (4.13 g, 21.98 mmol, 0.15 eq) in one portion under N₂. Then chlorotrimethylsilane (1.58 g, 14.66 mmol, 0.1 eq) was added slowly and the mixture was stirred for 30 min at 25° C. A solution of methyl 2-[(tert-butoxycarbonyl)amino]-3-iodopropanoate (57.86 g, 175.86 mmol, 1.2 eq) in DMA (80 mL) was added dropwise slowly (60 min) to maintain temperature below 50° C., the resulting mixture was stirred at rt for 2 h and then added 250 mL 3-necked round-bottom flask a cannula to a solution of 3-bromo-2-cyclopropoxy-5-fluoropyridine (34 g, 146.55 mmol, 1 equiv), Pd(dppf)Cl₂·CH₂Cl₂ (23.94 g, 29.31 mmol, 0.2 eq) and CuI (5.57 g, 29.31 mmol, 0.2 eq) in DMA (80 mL) under N₂, the color of the mixture turned brown, then the mixture was heated and stirred at 80° C. for 2 h under N₂. The mixture was quenched with ice-water (400 ml) and extracted with EA (3×500 ml). The organic layer was combined and washed with brine (300 ml), dried with anhydrous Na₂SO₄, filtered, and concentrated in vacuum to give the crude product. The crude product was purified by silica gel chromatography eluted with PE/EtOAc=0-50% to afford methyl (2S)-2-[(tert-butoxycarbonyl)amino]-3-(2-cyclopropoxy-5-fluoropyridin-3-yl)propanoate (11.2 g, 23%) as a yellow oil. LCMS (ESI+): m/z 355.10 (M+H⁺).

### Step 3: Int. 9

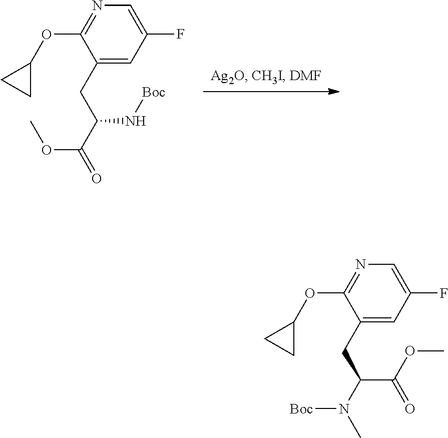 [SMILES: COC(=O)[C@H](Cc1cc(F)cnc1OC1CC1)NC(=O)OC(C)(C)C ; COC(=O)[C@H](Cc1cc(F)cnc1OC1CC1)N(C)C(=O)OC(C)(C)C]

Into a 1000 mL round-bottom flask were added methyl (2S)-2-[(tert-butoxycarbonyl)amino]-3-(2-cyclopropoxy-5-fluoropyridin-3-yl)propanoate (11 g, 31.07 mmol, 1 eq) in DMF (150 mL), Ag₂O (35.89 g, 155.37 mmol, 5 eq) was added at 0° C. under nitrogen atmosphere. The mixture was stirred at 0° C. for 30 min, methyl iodide (44.12 g, 310.73 mmol, 10 eq) was added dropwise over 2 min at 0° C. The resulting mixture was stirred at rt for 16 h. The reaction was quenched with ice-water (100 mL) and extracted with EtOAc (3×300 mL). The organic layer was combined and washed with brine (3×200 mL) and dried over anhydrous Na₂SO₄. After filtration, the filtrate was concentrated under reduced pressure. The residue was purified by silica gel column chromatography, eluted with PE/EA (7:1) to afford Int. 9 (10.8 g, 94%) as a yellow oil. LCMS (ESI+): m/z 369.15 (M+H⁺). ¹H NMR (400 MHz, DMSO-d₆): δ 8.06 (dd, J=18.7, 3.0 Hz, 1H), 7.55 (ddd, J=18.5, 8.7, 3.0 Hz, 1H), 4.68 (dd, J=10.6, 4.5 Hz, 1H), 4.24 (ddt, J=11.7, 8.6, 4.0 Hz, 1H), 3.68 (d, J=7.6 Hz, 3H), 3.13-2.84 (m, 2H), 2.63 (d, J=7.7 Hz, 3H), 1.24 (d, J=18.3 Hz, 9H), 0.80-0.63 (m, 4H).

### Int. 10: Preparation of (S)-2-((tert-butoxycarbonyl)(methyl)amino)-3-(5-chloro-2-isopropoxypyridin-3-yl)propanoic acid

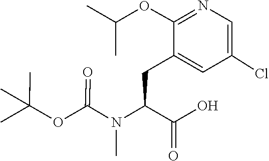 [SMILES: CC(C)Oc1ncc(Cl)cc1C[C@@H](C(=O)O)N(C)C(=O)OC(C)(C)C]

### Step 1: 3-bromo-5-chloro-2-isopropoxypyridine

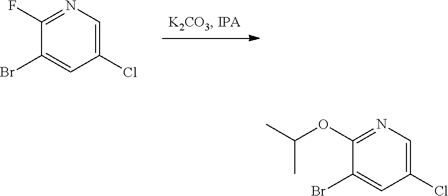

To a stirred mixture of 3-bromo-5-chloro-2-fluoropyridine (50 g, 237.61 mmol, 1 eq) and K₂CO₃ (98.52 g, 712.83 mmol, 3 eq) in IPA (500 mL, 5261.52 mmol, 22.14 eq) at 20° C. The resulting mixture was stirred for 48 h at 80° C. The resulting mixture was concentrated under vacuum and the mixture was dissolved in DCM (300 mL). The resulting mixture was filtered, and the filter cake was washed with DCM (3×50 mL). The filtrate was concentrated under reduced pressure. This resulted in 3-bromo-5-chloro-2-isopropoxypyridine (50 g, 84.0%) as a yellow oil. LCMS (ESI+): m/z 252.05 (M+H⁺).

### Step 2: Methyl (2S)-2-[(tert-butoxycarbonyl)amino]-3-(5-chloro-2-isopropoxypyridin-3-yl) propanoate

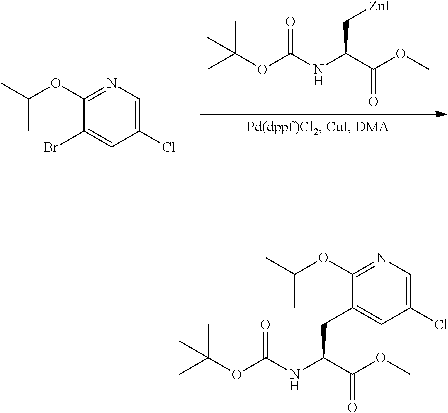

To a stirred mixture of 3-bromo-5-chloro-2-isopropoxypyridine (50 g, 199.59 mmol, 1 eq) and Pd(dppf)Cl₂ DCM adduct (16.26 g, 19.96 mmol, 0.1 eq), CuI (7.60 g, 39.92 mmol, 0.2 eq) in DMA (500 mL) was added methyl (2S)-2-[(tert-butoxycarbonyl)amino]-2-(iodozincio)acetate (162.6 mL, 199.59 mmol, 10 eq) dropwise at 20° C. under nitrogen atmosphere. The resulting mixture was stirred for 16 h at 80° C. under nitrogen atmosphere. The resulting mixture was filtered, the filter cake was washed with EtOAc (3×500 mL). The filtrate was extracted with EtOAc (3×800 mL). The combined organic layers were washed with H₂O (3×1000 mL) and dried over anhydrous Na₂SO₄. After filtration, the filtrate was concentrated under reduced pressure. The residue was purified by silica gel column chromatography, eluted with CH₂Cl₂/PE (5:1) to afford methyl (2S)-2-[(tert-butoxycarbonyl)amino]-3-(5-chloro-2-isopropoxypyridin-3-yl)propanoate (110 g) as a yellow oil. The residue was purified by reversed-phase flash chromatography with the following conditions: column, C18 silica gel; mobile phase, MeCN in Water (0.1% FA), 10% to 80% gradient in 35 min; detector, UV 280 nm. The resulting mixture was concentrated under vacuum. This resulted in methyl (2S)-2-[(tert-butoxycarbonyl)amino]-3-(5-chloro-2-isopropoxypyridin-3-yl) propanoate (53 g, 77%) as a white solid. LCMS (ESI+): m/z 373.25 (M+H⁺).

### Step 3: Methyl (2S)-2-[(tert-butoxycarbonyl)(methyl)amino]-3-(5-chloro-2-isopropoxypyridin-3-yl)propanoate

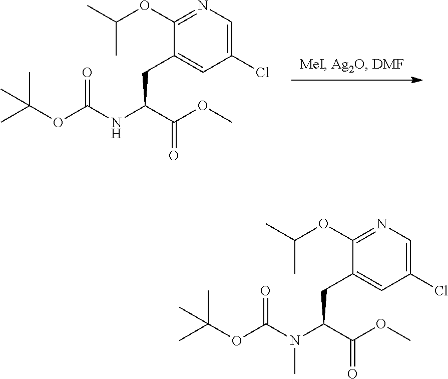

To a stirred mixture of methyl (2S)-2-[(tert-butoxycarbonyl)amino]-3-(5-chloro-2-isopropoxypyridin-3-yl)propanoate (20 g, 53.64 mmol, 1 eq) and Ag₂O (62.15 g, 268.21 mmol, 5 eq) in DMF (500 mL) was added MeI (77.66 g, 536.41 mmol, 10 eq) at 20° C. The resulting mixture was stirred for 16 h at 60° C. The resulting mixture was filtered, the filter cake was washed with EtOAc (3×500 mL). The filtrate was dissolved in H₂O (800 mL) and the mixture was extracted with EtOAc (3×1000 mL). The combined organic layers were washed with H₂O (4×1200 mL) and dried over anhydrous Na₂SO₄. After filtration, the filtrate was concentrated under reduced pressure to afford methyl (2S)-2-[(tert-butoxycarbonyl)(methyl)amino]-3-(5-chloro-2-isopropoxypyridin-3-yl)propanoate (20.5 g, 99%) as a yellow oil. LCMS (ESI+): m/z 387.15 (M+H⁺).

### Step 4: Int. 10

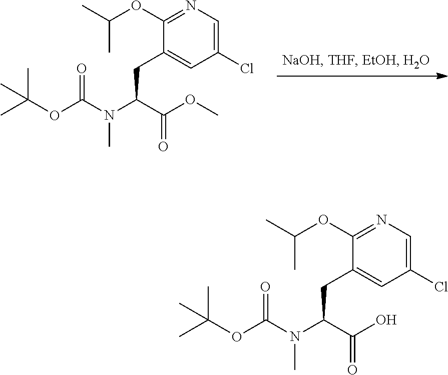

To a stirred mixture of methyl (2S)-2-[(tert-butoxycarbonyl)(methyl)amino]-3-(5-chloro-2-isopropoxypyridin-3-yl)propanoate (20 g, 51.70 mmol, 1 eq) in EtOH (30 mL) and THF (60 mL) were added NaOH (6.20 g, 155.09 mmol, 3 eq) in H₂O (120 mL) dropwise at 0° C. The resulting mixture was stirred for 16 h at 20° C. The resulting mixture was extracted with EtOAc (2×100 mL). The combined organic layers were washed with H₂O (1×150 mL). The combined water phase was acidified to pH=6 with diluted citric acid (1N). The resulting mixture was extracted with EtOAc (2×200 mL). The combined organic layers were washed with H₂O (1×100 mL) and dried over anhydrous Na₂SO₄. After filtration, the filtrate was concentrated under vacuum to afford Int. 10 (14.2 g, 74%) as a yellow oil. LCMS (ESI+): m/z 373.15 (M+H⁺).

Int. 11: Preparation of (S)-2-((tert-butoxycarbonyl)(methyl)amino)-3-(5-chloro-2-(pyrimidin-2-yloxy)phenyl)propanoic acid

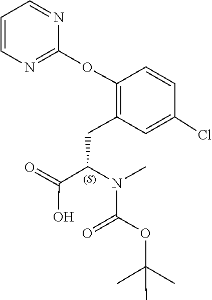

### Step 1: 5-(2-bromo-4-chlorophenyl)pyrimidine

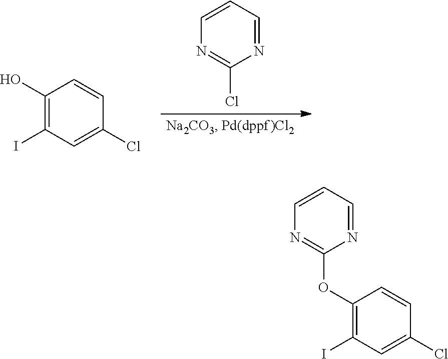

To a stirred solution of pyrimidin-5-ylboronic acid (10 g, 80.70 mmol, 1 eq) and 2-bromo-4-chloro-1-iodobenzene (25.61 g, 80.70 mmol, 1 eq) in 1,4-dioxane (80 mL) and H₂O (20 mL) were added Pd(dppf)Cl₂CH₂Cl₂ (6.57 g, 8.06 mmol, 0.10 eq) and Na₂CO₃ (25.66 g, 242.11 mmol, 3 eq) in portions at rt under nitrogen atmosphere. The resulting mixture was stirred for additional 4 h at 80° C. Desired product could be detected by LCMS. The resulting mixture was filtered, and the filter cake was washed with EtOAc (3×10 mL). The resulting mixture was diluted with water and extracted with EtOAc (3×50 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, MeCN in water (0.1% TFA), 5% to 100% gradient in 30 min; detector, UV 220 nm. This resulted in 5-(2-bromo-4-chlorophenyl)pyrimidine (9.6 g, 44%) as a brown oil. LCMS (ESI+): m/z 270.90 (M+H⁺).

### Step 2: Methyl (2S)-2-[(tert-butoxycarbonyl)amino]-3-[5-chloro-2-(pyrimidin-5-yl)phenyl]propanoate

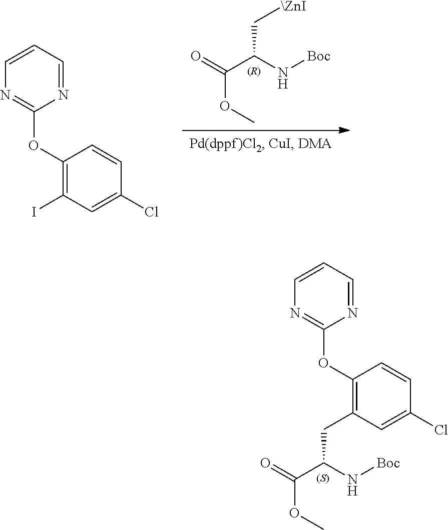

Into a stirred solution of Zn (6.31 g, 96.46 mmol, 2 eq) in DMA (30 mL) were added 1,2-dibromoethane (0.91 g, 4.82 mmol, 0.1 eq) and TMSCl (0.35 g, 3.23 mmol, 0.067 eq) dropwise at rt under nitrogen atmosphere. To the above mixture was added methyl (2R)-2-[(tert-butoxycarbonyl)amino]-3-iodopropanoate (15.87 g, 48.23 mmol, 1 eq) in DMA (30 mL) dropwise. The resulting mixture was stirred for additional 3 h at rt. To a stirred solution of 5-(2-bromo-4-chlorophenyl)pyrimidine (13 g, 48.23 mmol, 1 eq) in DMA (40 mL) were added Pd(dppf)Cl₂CH₂Cl₂ (3.93 g, 4.82 mmol, 0.1 eq) and CuI (1.84 g, 9.65 mmol, 0.2 eq) in portions at rt under nitrogen atmosphere. Then, the two mixtures were added together. The resulting mixture was stirred for additional 3 h at 80° C. Desired product could be detected by LCMS. The reaction was quenched by the addition of water (100 mL) at rt. The resulting mixture was filtered, and the filter cake was washed with EA (3×100 mL). The residue was diluted with water and EA. The organic phase was washed with brine (100 mL×3), dried over anhydrous sodium sulfate, and concentrated in vacuo. The residue was purified by reverse flash chromatography with the following conditions: column, C18 silica gel; mobile phase, MeCN in water (0.1% FA), 5% to 100% gradient in 30 min; detector, UV 220 nm. This resulted in methyl (2S)-2-[(tert-butoxycarbonyl)amino]-3-[5-chloro-2-(pyrimidin-5-yl)phenyl]propanoate (8 g, 42%) as a yellow solid. LCMS (ESI+): m/z 392.05 (M+H⁺).

### Step 3: Methyl(S)-2-((tert-butoxycarbonyl)(methyl)amino)-3-(5-chloro-2-(pyrimidin-2-yloxy)phenyl)propanoate

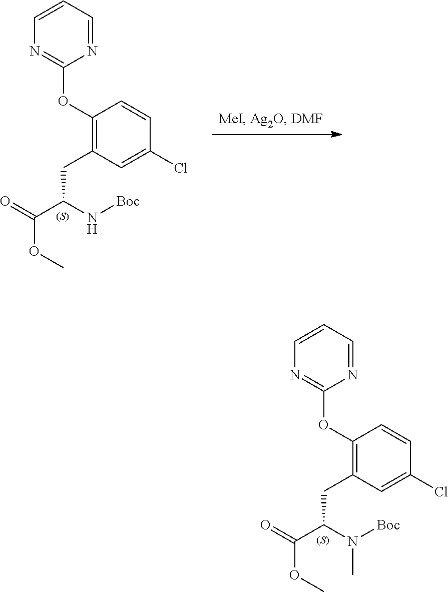

A solution of methyl (2S)-2-[(tert-butoxycarbonyl)amino]-3-[5-chloro-2-(pyrimidin-2-yloxy)phenyl]propanoate (1.3 g, 3.19 mmol, 1 eq) in DMF (15 mL) was treated with Ag₂O (3.69 g, 15.94 mmol, 5 eq) at rt under nitrogen atmosphere followed by the addition of CH₃I (6.79 g, 47.81 mmol, 15 eq) dropwise at rt. The resulting mixture was stirred for additional 3 h at rt. Desired product could be detected by LCMS. The resulting mixture was filtered, the filter cake was washed with DMF (30 mL). The filtrate was used in the next step directly without further purification. LCMS (ESI+): m/z 378.0 (M+H⁺).

### Step 4: Int. 11

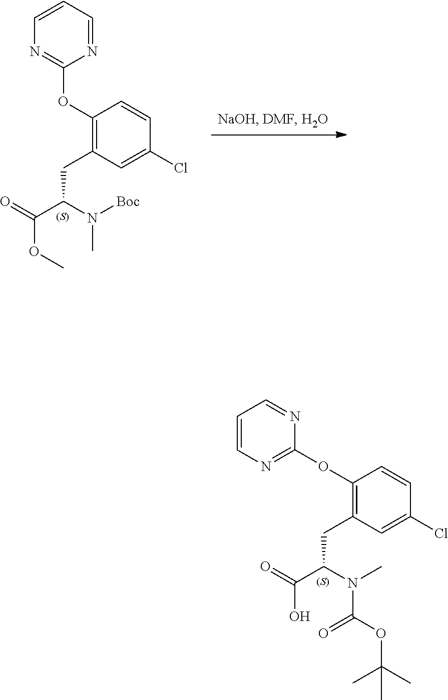

A solution of methyl (2S)-2-[(tert-butoxycarbonyl)(methyl)amino]-3-[5-chloro-2-(pyrimidin-2-yloxy)phenyl]propanoate (3 g, 7.111 mmol, 1 eq) in DMF (45 mL) and H₂O (10 mL) was treated with NaOH (0.85 g, 21.333 mmol, 3 eq) at rt. The resulting mixture was stirred for additional 16 h at rt. Desired product could be detected by LCMS. The resulting mixture was diluted with water (40 mL). The resulting mixture was extracted with EtOAc (3×50 mL). The combined organic layers were washed with brine (3×50 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 5% to 100% gradient in 10 min; detector, UV 220 nm. This resulted in Int. 11 (1.86 g, 62%) as a yellow solid. LCMS (ESI+): m/z 408.05 (M+H⁺).

### Int. 12: Preparation of (S)-2-((tert-butoxycarbonyl)(methyl)amino)-3-(5-chloro-2-cyclobutoxypyridin-3-yl)propanoic acid

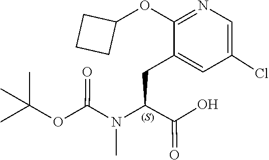

### Step 1: 3-Bromo-5-chloro-2-cyclobutoxypyridine

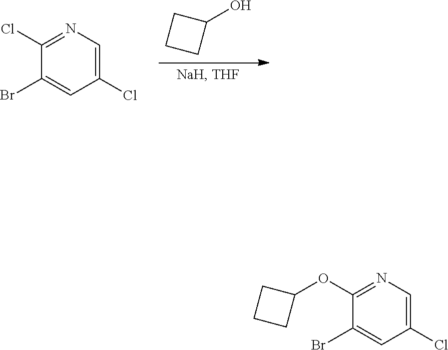

To a suspension of NaH (6.35 g, 158.67 mmol, 1.2 eq, 60% in oil) in THF (250 mL) was added cyclobutanol (10.49 g, 145.45 mmol, 1.1 eq) in THF (20 mL) at 0° C. dropwise. The resulting mixture was stirred for 1 h at 0° C. under nitrogen atmosphere. 3-bromo-2,5-dichloropyridine (30.0 g, 132.23 mmol, 1.0 eq) in THF (30 mL) was added and the mixture was stirred for 16 h at 60° C. under nitrogen atmosphere. The reaction was quenched by the addition of sat. NH₄Cl (aq.) (100 mL) at 0° C. The resulting mixture was diluted with water (500 mL) and then extracted with EtOAc (3×150 mL). The combined organic layers were washed with brine (1×300 mL), dried over anhydrous Na₂SO₄. After filtration, the filtrate was concentrated under reduced pressure. The residue was purified by silica gel column chromatography, eluted with PE/EA (40:1) to afford 3-bromo-5-chloro-2-cyclobutoxypyridine (31.0 g, 89%) as a colorless oil. LCMS (ESI+): m/z 262 (M+H⁺).

### Step 2: Methyl (2S)-2-[(tert-butoxycarbonyl)amino]-3-(5-chloro-2-cyclobutoxypyridin-3-yl) propanoate

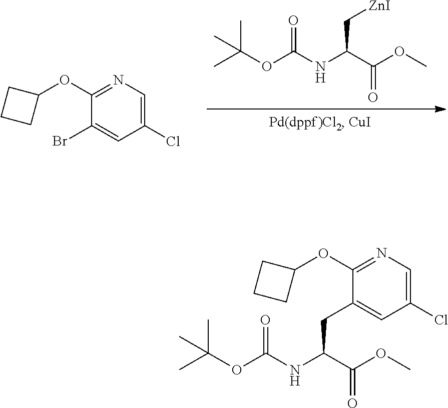

To a stirred mixture of 3-bromo-5-chloro-2-cyclobutoxypyridine (15 g, 57.14 mmol, 1.0 eq), Pd(dppf)Cl₂·CH₂Cl₂ (2.33 g, 2.86 mmol, 0.05 eq) and CuI (1.09 g, 5.71 mmol, 0.1 eq) in N, N-dimethylacetamide (300 mL) was added methyl (2R)-2-[(tert-butoxycarbonyl)amino]-3-(iodozincio) propanoate (171.41 mL, 171.41 mmol, 3.00 eq) in one portion at rt under nitrogen atmosphere. The resulting mixture was stirred for 16 h at 80° C. under nitrogen atmosphere. The resulting mixture was diluted with water (600 mL) and EtOAc (200 mL), and then filtered. The filter cake was washed with EtOAc (1×20 mL). The filtrate was separated, and the aqueous layer was extracted with EtOAc (2×200 mL). The combined organic layers were washed with sat. NH₄Cl (aq.) (2×300 mL), brine (1×300 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 10% to 80% gradient in 20 min; detector, UV 220 nm. This resulted in methyl (2S)-2-[(tert-butoxycarbonyl)amino]-3-(5-chloro-2-cyclobutoxypyridin-3-yl) propanoate (16.4 g, 75%) as a colorless oil. LCMS (ESI+): m/z 385 (M+H⁺).

### Step 3: Methyl (2S)-2-[(tert-butoxycarbonyl)(methyl)amino]-3-(5-chloro-2-cyclobutoxypyridin-3-yl) propanoate

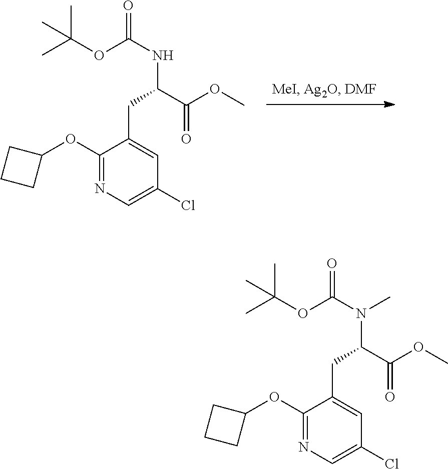

To a stirred mixture of methyl (2S)-2-[(tert-butoxycarbonyl)amino]-3-(5-chloro-2-cyclobutoxypyridin-3-yl) propanoate (16.4 g, 42.61 mmol, 1 eq) and Ag₂O (39.50 g, 170.45 mmol, 4.0 eq) in DMF (200 mL) was added CH₃I (96.8 g, 681.81 mmol, 16.0 eq) in one portion at rt under nitrogen atmosphere. The resulting mixture was stirred for 16 h at 50° C. under nitrogen atmosphere. The starting material was transformed completely. The resulting mixture was diluted with water (500 mL) and EtOAc (200 mL) and was then filtered. The filter cake was washed with EtOAc (1×20 mL). The resulting mixture was separated, and the aqueous layer was extracted with EtOAc (2×150 mL). The combined organic layers were washed with sat. NH₄Cl (aq.) (2×300 mL), brine (1×300 mL), dried over anhydrous Na₂SO₄. After filtration, the filtrate was concentrated under reduced pressure. The residue was purified by silica gel column chromatography, eluted with PE/EA (20:1) to afford methyl (2S)-2-[(tert-butoxycarbonyl)(methyl)amino]-3-(5-chloro-2-cyclobutoxypyridin-3-yl) propanoate (17.0 g, 95%) as a colorless oil.

### Step 4: Int. 12

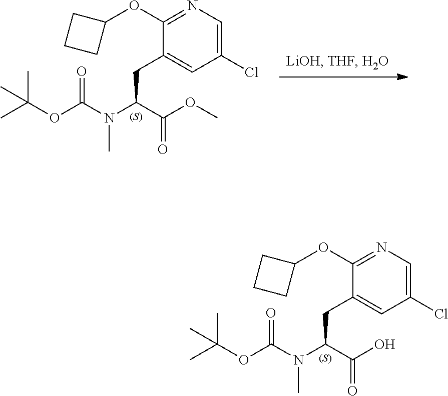

To a stirred solution of methyl (2S)-2-[(tert-butoxycarbonyl)(methyl)amino]-3-(5-chloro-2-cyclobutoxypyridin-3-yl) propanoate (17 g, 42.62 mmol, 1 eq) in THF (300 mL) and H₂O (100 mL) was added LiOH (3.06 g, 127.86 mmol, 3.00 eq) in H₂O (100 mL) dropwise at 0° C. The resulting mixture was stirred for 16 h at rt. The resulting mixture was diluted with water (300 mL) and then acidified to pH 2 with HCl (2 M aq.). The resulting mixture was extracted with EtOAc (3×150 mL). The combined organic layers were washed with brine (1×300 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 10% to 60% gradient in 25 min; detector, UV 220 nm. This resulted in Int. 12 (15.7 g, 95%) as a white solid. LCMS (ESI+): m/z 385 (M+H⁺). ¹H NMR (300 MHz, DMSO-d₆) δ 1.23 (d, J=21.1 Hz, 9H), 1.54-1.88 (m, 2H), 1.93-2.20 (m, 2H), 2.28-2.47 (m, 2H), 2.68 (d, J=1.4 Hz, 3H), 2.86-3.03 (m, 1H), 3.04-3.19 (m, 1H), 4.66-5.03 (m, 1H), 5.05-5.21 (m, 1H), 7.49-7.81 (m, 1H), 7.88-8.23 (m, 1H), 12.95 (s, 1H).

### Int. 13: Preparation of (S)-2-((tert-butoxycarbonyl)(methyl)amino)-3-(5-chloro-2-methoxypyridin-3-yl)propanoic acid

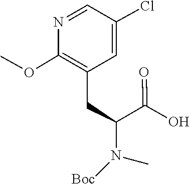 [SMILES: COc1ncc(Cl)cc1C[C@@H](C(=O)O)N(C)C(=O)OC(C)(C)C]

### Step 1: 5-Chloro-2-methoxy-3-iodopyridine

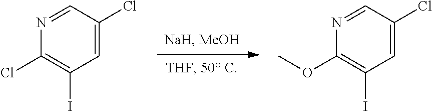

2,5-dichloro-3-iodopyridine (5.0 g, 1 eq, 18 mmol) solution in THF (26 mL) was brought to 0° C. and added NaH (1.5 g, 60% Wt, 2 eq, 37 mmol) as a solid. Then MeOH was added dropwise and stirred for 1 hour at 0° C. Then the mixture was stirred at 50° C. until LCMS analysis showed the complete consumption of starting material. The reaction was added into ice water 100 mL, then extracted with EtOAc (150 mL×2), the combined organic layers were washed with brine, dried over MgSO₄, and filtered. The filtrate was concentrated under reduced pressure to give crude product which was purified by column chromatography (SiO2, EtOAc:Hexanes; 0-60%) to yield 5-chloro-2-methoxy-3-iodopyridine (4.3 g, 16 mmol, 87% yield) as yellow solid.

### Step 2: Methyl(S)-2-((tert-butoxycarbonyl)amino)-3-(5-chloro-2-methoxypyridin-3-yl)propanoate

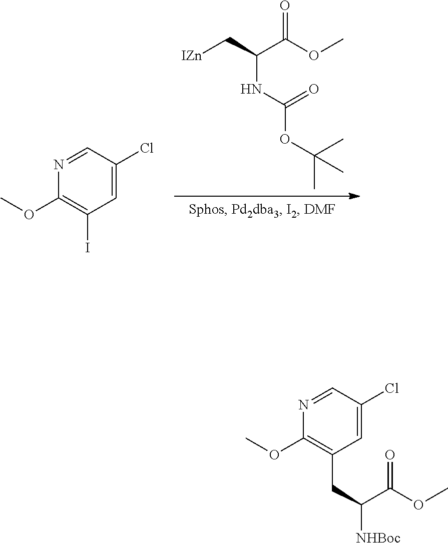

Zinc (2.4 g, 3 eq, 37 mmol) and DMF (15 mL) were added to a dry flask and degas with Ar. Then Boc-beta-iodo-Ala-OMe (4.0 g, 1 eq, 12 mmol) and iodine (0.47 g, 0.15 eq, 1.8 mmol) were added as solids. In a separate vial, 5-chloro-3-iodo-2-methoxypyridine (4.3 g, 1.3 eq, 16 mmol) and 2-Dicyclohexylphosphino-2,6-Dimethoxy-1,1-Biphenyl (0.13 g, 0.025 eq, 0.31 mmol) were dissolved in DMF (15 mL) and degassed by Ar. Then Pd₂dba₃ (0.14 g, 0.013 eq, 0.15 mmol) was added. Transfer the first solution into the second solution and stir at rt until LCMS analysis showed the complete consumption of starting material. The reaction was added into ice water 50 mL, then extracted with EtOAc (100 mL×2), the combined organic layers were washed with brine, dried over MgSO₄, and filtered. The filtrate was concentrated under reduced pressure to give crude product which was purified by column chromatography (SiO2, EtOAc:Hexanes; 0-60%) to yield methyl(S)-2-((tert-butoxycarbonyl)amino)-3-(5-chloro-2-methoxypyridin-3-yl)propanoate (1.8 g, 8.1 mmol, 66% yield) as white solid. LCMS (ESI+): m/z 345.3 (M+H⁺)

### Step 3: (S)-2-((tert-butoxycarbonyl)amino)-3-(5-chloro-2-methoxypyridin-3-yl)propanoic acid

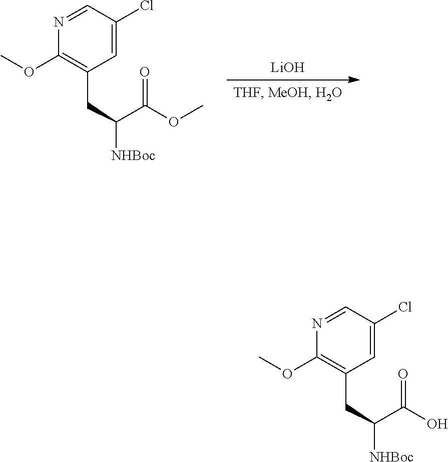

Methyl(S)-2-((tert-butoxycarbonyl)amino)-3-(5-chloro-2-methoxypyridin-3-yl)propanoate (2.8 g, 1 eq, 8.1 mmol) was dissolved into THF (20 mL), H₂O (14 mL) and MeOH (6.8 mL), then bring to 0° C. Lithium hydroxide monohydrate (1.0 g, 0.68 mL, 3 eq, 24 mmol) was added and the mixture was stirred at 25° C. until LCMS analysis showed the complete consumption of starting material. Then the 10% citric acid was added to the mixture at 0° C. until pH was acidic. The reaction mixture was concentrated via rotavapor and extracted with EtOAc (80 mL×2), the combined organic layers were washed with brine, dried over MgSO₄ and filtered. The filtrate was concentrated under reduced pressure to give crude product which was purified by column chromatography to give (S)-2-((tert-butoxycarbonyl)amino)-3-(5-chloro-2-methoxypyridin-3-yl)propanoic acid (2.3 g, 7.0 mmol, 86% yield). LCMS (ESI+): m/z 331.4 (M+H⁺)

### Step 4: Int. 13

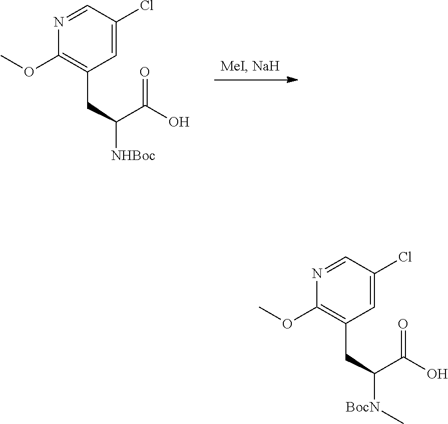

(S)-2-((tert-butoxycarbonyl)amino)-3-(5-chloro-2-methoxypyridin-3-yl)propanoic acid (2.3 g, 1 eq, 7.0 mmol) solid was dissolved in THF (35 mL) and brought to 0° C. under Ar. Then NaH (0.56 g, 60% Wt, 2 eq, 14 mmol) was added as solid, and MeI (5.9 g, 2.6 mL, 6 eq, 42 mmol) was added dropwise. The mixture was stirred for 1 hour at 0° C. Then the mixture was stirred at 25° C. until LCMS analysis showed the complete consumption of starting material. The reaction was quenched with ice water 100 mL and 10% citric acid was added to the mixture at 0° C. until pH was acidic. The reaction mixture was concentrated via rotavapor and extracted with EtOAc (80 mL×2), the combined organic layers were washed with brine, dried over MgSO₄, and filtered. The filtrate was concentrated under reduced pressure to give crude product which was purified by column chromatography to give Int. 13 (2.3 g, 6.7 mmol, 96% yield) as white solid. LCMS (ESI+): m/z 345.3 (M+H⁺).

### Int. 14: (S)-2-((tert-butoxycarbonyl)(methyl)amino)-3-(5-chloro-2-ethoxypyridin-3-yl)propanoic acid

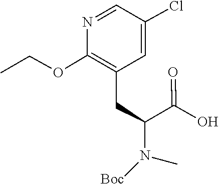 [SMILES: CCOc1ncc(Cl)cc1C[C@@H](C(=O)O)N(C)C(=O)OC(C)(C)C]

This material was synthesized by following the procedure for Int. 13 with ethanol as the starting alcohol. LCMS (ESI+): m/z 359.3 (M+H⁺)

### Int. 15: (S)-2-((tert-butoxycarbonyl)(methyl)amino)-3-(5-chloro-2-isobutoxypyridin-3-yl)propanoic acid

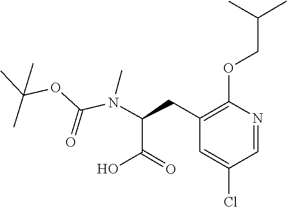 [SMILES: CC(C)COc1ncc(Cl)cc1C[C@@H](C(=O)O)N(C)C(=O)OC(C)(C)C]

This material was synthesized by following the procedure for Int. 13 with 2-methylpropan-1-ol as the starting alcohol. LCMS (ESI+): m/z 387.3 (M+H⁺)

### Int. 16: Preparation of (S)-2-((tert-butoxycarbonyl)(methyl)amino)-3-(5-chloro-2-(5-cyclopropyl-1,3,4-oxadiazol-2-yl)phenyl)propanoic acid

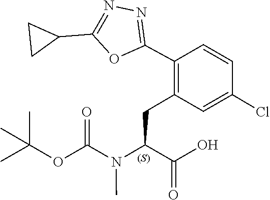

### Step 1: 4-Chloro-N′-cyclopropanecarbonyl-2-iodobenzohydrazide

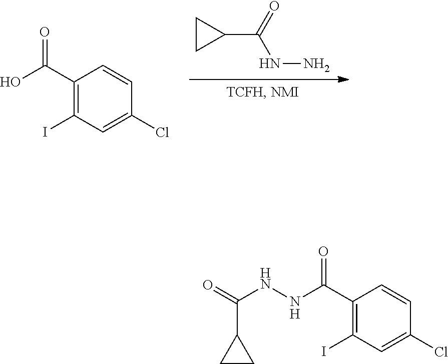

To a stirred solution of 4-chloro-2-iodobenzoic acid (1 g, 3.54 mmol, 1 eq) and NMI (0.87 g, 10.62 mmol, 3 eq) in ACN (40 mL) were added cyclopropanecarbohydrazide (354.45 mg, 3.540 mmol, 1 eq) and TCFH (1.19 g, 4.25 mmol, 1.2 eq) in portions at 0° C. The resulting mixture was stirred for 16 h at rt. The resulting mixture was diluted with water (20 mL) and extracted with EtOAc (3×50 mL). The combined organic layers were washed with brine (1×50 mL) and dried over anhydrous Na₂SO₄. After filtration, the filtrate was concentrated under vacuum. The residue was purified by reversed-phase flash chromatography with the following conditions: column, C18 silica gel; mobile phase, Acetonitrile in Water (0.1% FA), 0% to 60% gradient in 30 min; detector, UV 254 nm. This resulted in 4-chloro-N′-cyclopropanecarbonyl-2-iodobenzohydrazide (815 mg, 62%) as an off-white solid. LCMS (ESI+): m/z 364.80 (M+H⁺).

### Step 2: 2-(4-chloro-2-iodophenyl)-5-cyclopropyl-1,3,4-oxadiazole

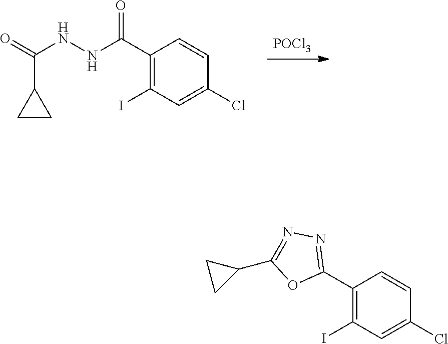 [SMILES: O=C(NNC(=O)C1CC1)c1ccc(Cl)cc1I ; "POCl3" ; Clc1ccc(-c2nnc(C3CC3)o2)c(I)c1]

A solution of 4-chloro-N′-cyclopropanecarbonyl-2-iodobenzohydrazide (5 g, 13.72 mmol, 1 eq) in POCl₃ (100 mL) was stirred for 2 h at 85° C. The resulting mixture was concentrated under vacuum and extracted with CH₂Cl₂ (3×200 mL). The combined organic layers were washed with NaHCO₃ (aq.) (1×200 mL) and dried over anhydrous Na₂SO₄. After filtration, the filtrate was concentrated under vacuum to afford 2-(4-chloro-2-iodophenyl)-5-cyclopropyl-1,3,4-oxadiazole (4.5 g, 82%) as a brown solid. The crude product was used in the next step directly without further purification. LCMS (ESI+): m/z 347.00 (M+H⁺).

### Step 3: Methyl (2S)-2-[(tert-butoxycarbonyl)amino]-3-[5-chloro-2-(5-cyclopropyl-1,3,4-oxadiazol-2-yl)phenyl]propanoate

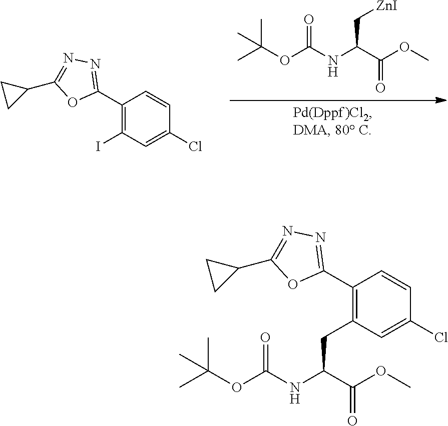

To a stirred solution of Zn (4.98 g, 76.18 mmol, 2.4 eq), 1,2-Dibromoethane (609.07 mg, 3.17 mmol, 0.1 eq) in DMA (20 mL) was added TMSCl (231.04 mg, 2.13 mmol, 0.067 eq) dropwise at 25° C. under nitrogen atmosphere. The resulting mixture was stirred for 30 min and methyl (2R)-2-[(tert-butoxycarbonyl)amino]-3-iodopropanoate (20.89 g, 63.48 mmol, 2.00 eq) in DMA (20 mL) was added to the above solution. The temperature risen and kept it under 50° C. After 30 min, the mixture was added to a solution of 2-(4-chloro-2-iodophenyl)-5-cyclopropyl-1,3,4-oxadiazole (11 g, 31.74 mmol, 1 eq), CuI (1.21 g, 6.35 mmol, 0.2 eq) and Pd(dppf)Cl₂ (2.32 g, 3.174 mmol, 0.1 equiv.) in DMA (200 mL) dropwise and stirred for 2 h at 80° C. The reaction was quenched with sat. NH₄Cl (aq.) at 0° C. The resulting mixture was filtered, the filter cake was washed with CH₂Cl₂ (500 mL) and the filtrate was extracted with CH₂Cl₂ (1000 mL). The combined organic layers were washed with brine (3×200 mL) and dried over anhydrous Na₂SO₄. After filtration, the filtrate was concentrated under vacuum. The residue was purified by reverse flash chromatography with the following conditions: column, C18 silica gel; mobile phase, Acetonitrile in Water (0.1% FA), 0% to 100% gradient in 40 min; detector, UV 254 nm. This resulted in methyl (2S)-2-[(tert-butoxycarbonyl)amino]-3-[5-chloro-2-(5-cyclopropyl-1,3,4-oxadiazol-2-yl)phenyl]propanoate (7.2 g, 197%) as a brown solid. LCMS (ESI+): m/z 422.20 (M+H⁺).

### Step 4: Methyl (2S)-2-[(tert-butoxycarbonyl)(methyl)amino]-3-[5-chloro-2-(5-cyclopropyl-1,3,4-oxadiazol-2-yl)phenyl]propanoate

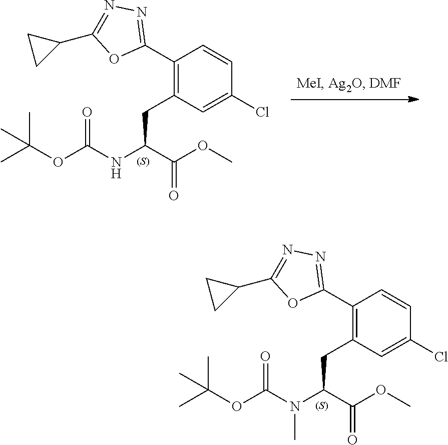

To a stirred solution of methyl (2S)-2-[(tert-butoxycarbonyl)amino]-3-[5-chloro-2-(5-cyclopropyl-1,3,4-oxadiazol-2-yl)phenyl]propanoate (7 g, 16.59 mmol, 1 eq) and Ag₂O (15.38 g, 66.37 mmol, 4 eq) in DMF (140 mL) was added CH₃I (23.55 g, 165.9 mmol, 10 eq) dropwise at 0° C. The resulting mixture was stirred for 72 h at rt. The resulting mixture was filtered, the filter cake was washed with EtOAc (3×100 mL) and the filtrate was extracted with EtOAc (3×100 mL). The combined organic layers were washed with brine (3×200 mL) and dried over anhydrous Na₂SO₄. After filtration, the filtrate was concentrated under vacuum. The crude product (7 g) was purified by Prep-HPLC with the following conditions (Column: XBridge C18 OBD Prep Column, 100 Å 10 μm, 19 mm×250 mm; Mobile Phase A: Water (10 mmol/L NH₄HCO₃), Mobile Phase B: ACN; Flow rate: 60 mL/min; Gradient: 60% B to 60% B in 24 min; Wave Length: 220 nm; RT1 (min): 18; Number Of Runs: 0) to afford methyl (2S)-2-[(tert-butoxycarbonyl)(methyl)amino]-3-[5-chloro-2-(5-cyclopropyl-1,3,4-oxadiazol-2-yl)phenyl]propanoate (3.6 g, 50%) as a brown oil. LCMS (ESI+): m/z 565.60 (M+H⁺).

### Step 5: Int. 16

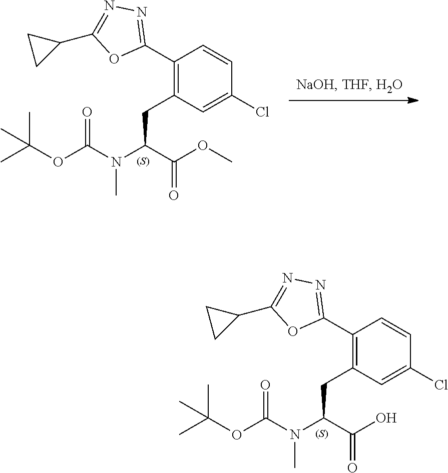

To a stirred solution of methyl (2S)-2-[(tert-butoxycarbonyl)(methyl)amino]-3-[5-chloro-2-(5-cyclopropyl-1,3,4-oxadiazol-2-yl)phenyl]propanoate (2.6 g, 5.97 mmol, 1 eq) in THF (24 mL)/H2O (8 mL) was added NaOH (715.69 mg, 17.90 mmol, 3.00 eq) in water (10 mL) dropwise at 0° C. The mixture was acidified to pH=5 with HCl (1N). The resulting mixture was extracted with EtOAc (3×200 mL). The combined organic layers were washed with brine (1×50 mL) and dried over anhydrous Na₂SO₄. After filtration, the filtrate was concentrated under vacuum. The residue was purified by reversed-phase flash chromatography with the following conditions: column, C18 silica gel; mobile phase, Acetonitrile in Water (0.1% FA), 5% to 100% gradient in 30 min; detector, UV 254 nm. This resulted in Int. 16 (2.73 g, 108%) as an off-white solid. LCMS (ESI+): m/z 421.95 (M+H⁺).

### Int. 17: Preparation of (S)-3-(2-((1-acetylpiperidin-4-yl)oxy)-5-chlorophenyl)-2-((tert-butoxycarbonyl)(methyl)amino)propanoic acid

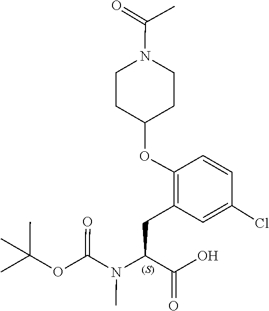

### Step 1: 1-(4-(4-chloro-2-iodophenoxy)piperidin-1-yl)ethan-1-one

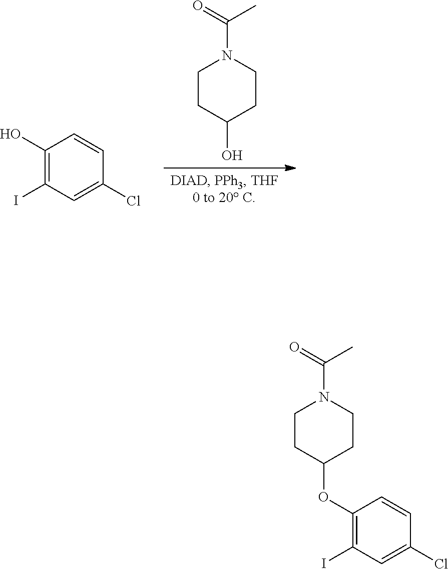

To a solution of 4-chloro-2-iodophenol (20 g, 78.60 mmol, 1 eq), 1-(4-hydroxy-1-piperidyl)ethanone (12.38 g, 86.46 mmol, 1.1 eq) and PPh₃ (24.74 g, 94.32 mmol, 1.2 eq) in THF (200 mL) was added DIAD (19.07 g, 94.32 mmol, 18.34 mL, 1.2 eq) dropwise at 0° C. under N₂ atmosphere. Then the reaction mixture was allowed to warm to 20° C. slowly and stirred at 20° C. for 12 h. LCMS showed starting material was consumed completely and desired mass was detected. The reaction mixture was quenched by water (200 mL), extracted with EtOAc (150 mL×2). The combined organics were washed with brine (150 mL), dried over Na₂SO₄, filtered, and concentrated under reduced pressure to give a residue. The residue was purified by flash silica gel chromatography (ISCO®; 330 g SepaFlash® Silica Flash Column, Eluent of 0-55% EtOAc/PE gradient@200 mL/min) to give 1-(4-(4-chloro-2-iodophenoxy)piperidin-1-yl)ethan-1-one (21.49 g, 39.55 mmol, 50% yield, 70% purity) as a light-yellow solid.

### Step 2: methyl(S)-3-(2-((1-acetylpiperidin-4-yl)oxy)-5-chlorophenyl)-2-((tert-butoxycarbonyl)amino)propanoate

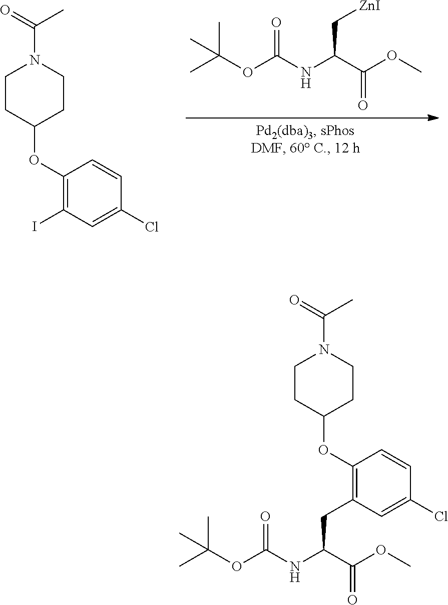

A mixture of Zn (6.95 g, 106.34 mmol, 3.5 eq) in DMF (50 mL) was degassed and purged with N₂ for 3 times, and the mixture was stirred at 120° C. for 10 min under N₂ atmosphere. Then I₂ (2.31 g, 9.11 mmol, 1.84 mL, 0.3 eq) in DMF (10 mL) was added dropwise at 20° C., methyl (R)-2-((tert-butoxycarbonyl)amino)-3-iodopropanoate (10 g, 30.38 mmol, 1 eq) in DMF (30 mL) was added dropwise, I₂ (2.31 g, 9.11 mmol, 1.84 mL, 0.3 eq) in DMF (10 mL) was added dropwise, the reaction mixture was stirred at 20° C. for 1 hr. TLC (PE:EtOAc=5:1, Rf=0.5) showed starting material was consumed completely and a new major spot was detected. The (R)-(2-((tert-butoxycarbonyl)amino)-3-methoxy-3-oxopropyl)zinc(II) iodide (11.99 g, crude) as a gray liquid was obtained, which was used into the next step without further purification.

To a solution of 1-(4-(4-chloro-2-iodophenoxy)piperidin-1-yl)ethan-1-one (5.58 g, 14.70 mmol, 1 eq), sPhos (603.43 mg, 1.47 mmol, 0.1 eq) and Pd₂(dba)₃ (673.00 mg, 734.95 umol, 0.05 eq) in DMF (50 mL) was degassed and purged with N₂ 3 times. Then (R)-(2-((tert-butoxycarbonyl)amino)-3-methoxy-3-oxopropyl)zinc(II) iodide (11.99 g, 30.39 mmol, 2.07 eq) in DMF was added dropwise. Then, the reaction mixture was stirred at 65° C. for 12 h. LCMS showed starting material was consumed completely and desired mass was detected. The reaction mixture was cooled to rt and quenched by water (150 mL), extracted with EtOAc (100 mL×2). The combined organics were washed with brine (150 mL), dried over Na₂SO₄, filtered, and concentrated under reduced pressure to give a residue. The residue was purified by prep-HPLC (column: 800 g Agela C18; mobile phase: [water-ACN]; B %: 20-50% 30 min; 50% 20 min) to give methyl(S)-3-(2-((1-acetylpiperidin-4-yl)oxy)-5-chlorophenyl)-2-((tert-butoxycarbonyl)amino)propanoate (3.62 g, 7.66 mmol, 52% yield, 96% purity) as a white solid.

### Step 3: (S)-3-(2-((1-acetylpiperidin-4-yl)oxy)-5-chlorophenyl)-2-((tert-butoxycarbonyl)amino)propanoic acid

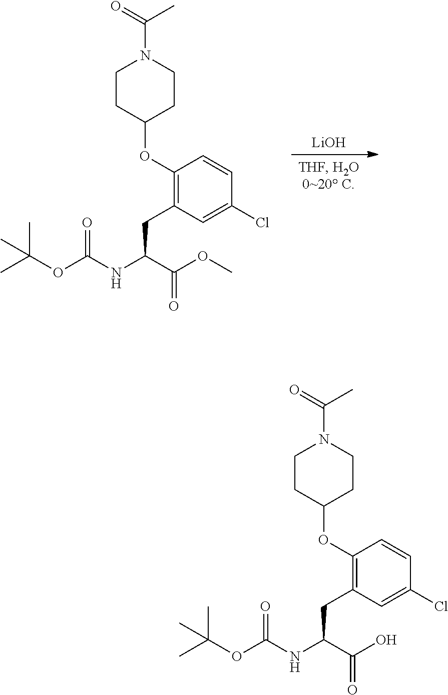

To a solution of methyl(S)-3-(2-((1-acetylpiperidin-4-yl)oxy)-5-chlorophenyl)-2-((tert-butoxycarbonyl)amino)propanoate (3.62 g, 7.96 mmol, 1 eq) in THF (40 mL) was added a solution of LiOH·H₂O (400.69 mg, 9.55 mmol, 1.2 eq) in H₂O (20 mL). Then the reaction mixture was stirred at 25° C. for 1 hr. LCMS showed starting material was consumed completely and desired mass was detected. The reaction mixture was concentrated under reduced pressure to remove THF. Then the mixture was acidified by 1N HCl to pH=2-3 at 0° C. and extracted with EtOAc (15 mL×2). The combined organics were washed with brine (20 mL), dried over Na₂SO₄, filtered, and concentrated under reduced pressure to give compound (S)-3-(2-((1-acetylpiperidin-4-yl)oxy)-5-chlorophenyl)-2-((tert-butoxycarbonyl)amino)propanoic acid (3.35 g, 7.60 mmol, 95% yield, 100% purity) as a white solid.

### Step 4: Int. 17

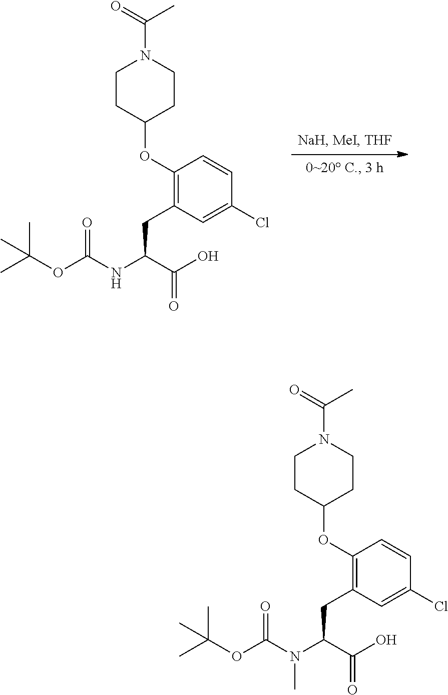

To a solution of (S)-3-(2-((1-acetylpiperidin-4-yl)oxy)-5-chlorophenyl)-2-((tert-butoxycarbonyl)amino)propanoic acid (3.35 g, 7.60 mmol, 1 eq) in THF (40 mL) was added NaH (759.71 mg, 18.99 mmol, 60% purity, 2.5 eq) at 0° C. under N₂ atmosphere. The mixture was stirred at 20° C. for 30 min. Then MeI (14.02 g, 98.77 mmol, 6.15 mL, 13 eq) was added at 0° C. and the reaction mixture was stirred at 20° C. for 12 hr. LCMS showed 38% of starting material remained and 26% of desired mass was detected. The reaction mixture was cooled to 0° C. and quenched by NH₄Cl (40 mL), extracted with EtOAc (30 mL×2). The combined organics were washed with brine (30 mL), dried over Na₂SO₄, filtered, and concentrated under reduced pressure to give a residue. The crude product was purified by reversed-phase HPLC (column: 800 g Agela C18; mobile phase: [water (FA)-ACN]; B %: 25-55% 30 min; 55% 20 min) to Int. 17 (3.02 g, 6.41 mmol, 97% purity) was obtained as a light-yellow solid. Spectrum: ¹H NMR (400 MHz, DMSO-d₆) δ=12.80 (br s, 1H), 7.28-6.99 (m, 3H), 4.90-4.60 (m, 2H), 3.73-3.54 (m, 2H), 3.39 (td, J=3.8, 13.3 Hz, 2H), 3.18 (br dd, J=3.8, 13.8 Hz, 1H), 2.95-2.82 (m, 1H), 2.66 (s, 3H), 2.01 (s, 3H), 1.97-1.80 (m, 2H), 1.74-1.50 (m, 2H), 1.30-1.15 (m, 9H)

### Int. 18: Preparation of (S)-2-((tert-butoxycarbonyl)(methyl)amino)-3-(5-fluoro-2-(1-methyl-1H-pyrazol-4-yl)pyridine-3-yl)propanoic acid

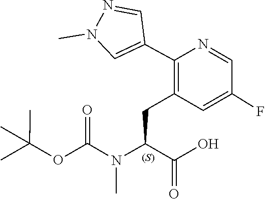

### Step 1: 5-Fluoro-2-(1-methyl-1H-pyrazol-4-yl)pyridine-3-amine

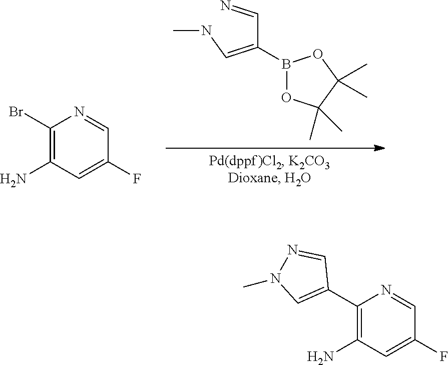

To a solution of 2-bromo-5-fluoropyridin-3-amine (12 g, 62.83 mmol, 1 eq) in dioxane (96 mL) and H₂O (18 mL) was added, 1-methyl-4-(4,4,5,5-tetramethyl-1,3,2-dioxaborolan-2-yl)-1H-pyrazole, Pd(dppf)Cl₂ (2.30 g, 3.14 mmol, 0.05 eq) and K₂CO₃ (17.37 g, 125.65 mmol, 2 eq), the mixture was stirred at 90° C. for 12 h under N₂. The reaction was monitored by LCMS, which showed 2-bromo-5-fluoropyridin-3-amine was consumed and one of peak with desired mass was detected. Five other reactions of the same size were set up and all six reactions were combined to work up. After cooling to rt, the mixture was filtered. The filtrate was diluted with water (500 mL), extracted with EtOAc (200 mL×3). The combined organics were washed with brine (300 mL), dried over Na₂SO₄, filtered, and concentrated under reduced pressure to give a residue. The residue was dissolved in DCM (200 mL), and 250 g of silica gel was added. The resulting mixture was evaporated under reduced pressure to give a dry flowing solid, and then it was loaded to Biotage® using a 400 g Agela flash silica gel column, eluted with 0% to 50% EtOAc in PE with the flower rate of 75 mL/min. The product fraction was combined and concentrated to give 5-fluoro-2-(1-methyl-1H-pyrazol-4-yl)pyridine-3-amine (63 g, crude) as a yellow solid.

### Step 2: 5-Fluoro-3-iodo-2-(1-methyl-1H-pyrazol-4-yl)pyridine

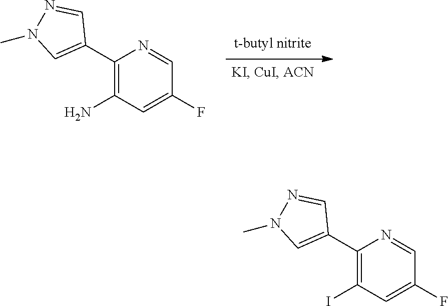

To a solution of 5-fluoro-2-(1-methyl-1H-pyrazol-4-yl)pyridine-3-amine (15.75 g, 81.95 mmol, 1 eq) in MeCN (450 mL) was added CuI (18.73 g, 98.34 mmol, 1.2 eq), KI (68.02 g, 409.75 mmol, 5 eq) and tert-butyl nitrite (42.25 g, 409.75 mmol, 48.73 mL, 5 eq), the mixture was stirred at 60° C. for 12 h under N₂. The reaction was monitored by LCMS, which showed 5-fluoro-2-(1-methyl-1H-pyrazol-4-yl)pyridine-3-amine as consumed and one of peak with desired mass was detected. Three other reactions of the same size were set up and all four reactions were combined and filtered, and the filter cake was rinsed with EtOAc (100 mL×3). The combined filtrate were concentrated under reduced pressure to give a residue. The filtrate was diluted with water (200 mL), extracted with dichloromethane (80 mL×3). The combined organics were washed with brine (100 mL), dried over Na₂SO₄, filtered, and concentrated under reduced pressure to give a residue. The residue was dissolved in DCM (100 mL), and 200 g of silica gel was added. The resulting mixture was evaporated under reduced pressure to give a dry flowing solid, and then it was loaded to Biotage® using a 300 g Agela flash silica gel column, eluted with 0% to 20% EtOAc in PE with the flower rate of 75 mL/min. The product fraction was combined and concentrated to give 5-fluoro-3-iodo-2-(1-methyl-1H-pyrazol-4-yl)pyridine (30 g, 98.99 mmol, 60% yield, 100% purity) as a yellow solid.

### Step 3: Methyl(S)-2-((tert-butoxycarbonyl)amino)-3-(5-fluoro-2-(1-methyl-1H-pyrazol-4-yl)pyridine-3-yl)propanoate

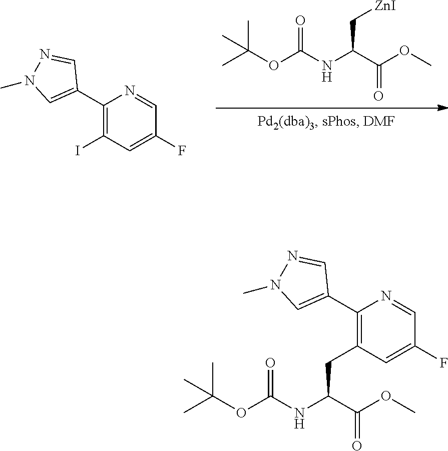

To a solution of 5-fluoro-3-iodo-2-(1-methyl-1H-pyrazol-4-yl)pyridine (30 g, 98.99 mmol, 1 eq), SPhos (4.06 g, 9.90 mmol, 0.1 eq) and Pd₂(dba)₃ (4.83 g, 5.28 mmol, 0.05 eq) in DMF (330 mL) was degassed and purged with N₂ 3 times. Then (R)-(2-((tert-butoxycarbonyl)amino)-3-methoxy-3-oxopropyl)zinc(II) iodide (78.10 g, 197.97 mmol, 2 eq) in DMF (200 mL) was added dropwise. Then the reaction mixture was stirred at 20° C. for 12 h. The reaction was monitored by LCMS, which showed 5-fluoro-3-iodo-2-(1-methyl-1H-pyrazol-4-yl)pyridine was consumed and one of peak with desired mass was detected. The mixture was filtered. The filtrate was diluted with water (200 mL), extracted with EtOAc (100 mL×3). The combined organics were washed with brine (200 mL), dried over Na₂SO₄, filtered, and concentrated under reduced pressure to give a residue. The residue was purified by preparative HPLC (column: Welch Xtimate C18 250×100 mm #10 um; mobile phase: [water (NH₄HCO₃)-ACN]; B %: 30%-60%, 36 min) the HPLC fractions were combined, lyophilized. The product fraction was combined and concentrated to give methyl(S)-2-((tert-butoxycarbonyl)amino)-3-(5-fluoro-2-(1-methyl-1H-pyrazol-4-yl)pyridine-3-yl)propanoate (4.2 g, 11.10 mmol, 11% yield, 100% purity) as a yellow solid.

### Step 4: (S)-2-((tert-butoxycarbonyl)amino)-3-(5-fluoro-2-(1-methyl-1H-pyrazol-4-yl)pyridine-3-yl)propanoic acid

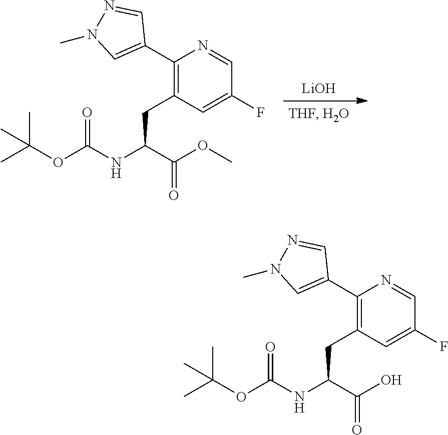

To a solution of methyl(S)-2-((tert-butoxycarbonyl)amino)-3-(5-fluoro-2-(1-methyl-1H-pyrazol-4-yl)pyridine-3-yl)propanoate (4.2 g, 11.10 mmol, 1 eq) in THF (60 mL) was added a solution of LiOH·H₂O (558.93 mg, 13.32 mmol, 1.2 eq) in H₂O (30 mL), the mixture was stirred at 20° C. for 1 h. The mixture was acidified by adding hydrochloric acid (0.1 M, 10 mL) dropwise at 20° C. to pH=5. The filtrate was diluted with water (30 mL), extracted with EtOAc (20 mL×3). The combined organics were washed with brine (20 mL), dried over Na₂SO₄, filtered, and concentrated under reduced pressure to give (S)-2-((tert-butoxycarbonyl)amino)-3-(5-fluoro-2-(1-methyl-1H-pyrazol-4-yl)pyridine-3-yl)propanoic acid (3.7 g, 10.15 mmol, 91% yield, 100% purity) as a yellow solid.

### Step 5: Int. 18

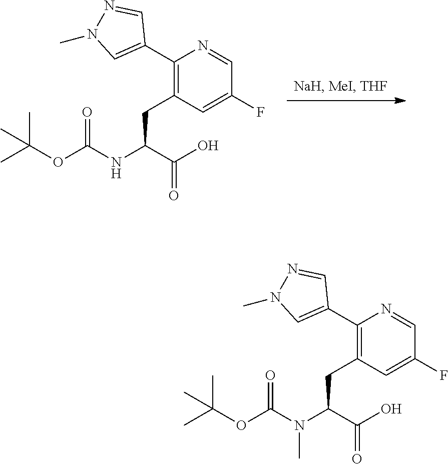

To a solution of (S)-2-((tert-butoxycarbonyl)amino)-3-(5-fluoro-2-(1-methyl-1H-pyrazol-4-yl)pyridine-3-yl)propanoic acid (1.85 g, 5.08 mmol, 1 eq) in THF (76 mL) was added NaH (1.02 g, 25.39 mmol, 60% purity, 5 eq) at 0° C., the mixture was stirred at 0° C. for 1 h under N₂. MeI (7.21 g, 50.77 mmol, 3.16 mL, 10 eq) was added to the reaction mixture, the mixture was stirred at 20° C. for 12 h under N₂. LCMS showed the (S)-2-((tert-butoxycarbonyl)(methyl)amino)-3-(5-fluoro-2-(1-methyl-1H-pyrazol-4-yl)pyridine-3-yl)propanoic acid was consumed and desired mass was detected. One other reaction of the same size was set up and all two reactions were combined to work up. The reaction was quenched by addition of saturated ammonium chloride aqueous solution (20 mL), acidified by adding hydrochloric acid (0.1 M, 10 mL) dropwise at 0° C. to pH=5, extracted with EtOAc (20 mL×3). The combined organics were washed with brine (30 mL), dried over Na₂SO₄, filtered, and concentrated under reduced pressure to give Int. 18 (2.5 g, 6.53 mmol, 64.3% yield, 99% purity) as a yellow solid. ¹H NMR (400 MHz, MeOD −d4) δ=8.34 (dd, J=2.4, 15.3 Hz, 1H), 8.02 (d, J=7.0 Hz, 1H), 7.84 (d, J=3.1 Hz, 1H), 7.52 (dt, J=2.6, 9.2 Hz, 1H), 4.74-4.62 (m, 1H), 4.02-3.92 (m, 3H), 3.64-3.54 (m, 1H), 3.28-3.12 (m, 1H), 2.66 (d, J=6.9 Hz, 3H), 1.42-1.18 (m, 9H).

### Int. 19: Preparation of (S)-2-((tert-butoxycarbonyl)(methyl)amino)-3-(5-chloro-2-(1-methyl-1H-pyrazol-4-yl)pyridine-3-yl)propanoic acid

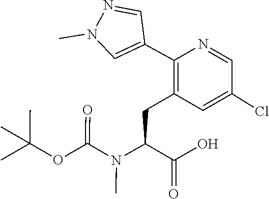 [SMILES: CN(C(=O)OC(C)(C)C)[C@@H](Cc1cc(Cl)cnc1-c1cnn(C)c1)C(=O)O]

### Step 1: 5-chloro-2-(1-methyl-1H-pyrazol-4-yl)pyridine-3-amine

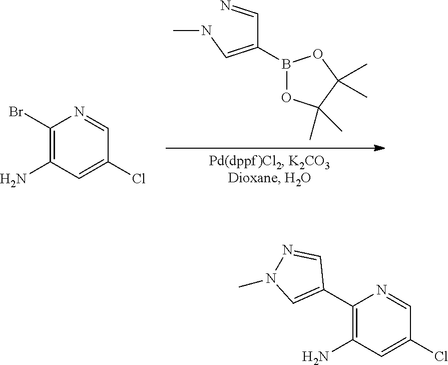

To a solution of 2-bromo-5-chloropyridin-3-amine (30 g, 144.61 mmol, 1 eq) and 1-methyl-4-(4,4,5,5-tetramethyl-1,3,2-dioxaborolan-2-yl)-1H-pyrazole (36.11 g, 173.53 mmol, 1.2 eq) in dioxane (720 mL) and H₂O (120 mL) was added K₂CO₃ (39.97 g, 289.22 mmol, 2 eq) and Pd(dppf)Cl₂ (3.17 g, 4.34 mmol, 0.03 eq), the mixture was stirred at 90° C. for 12 h under N₂. The reaction was monitored by LCMS, which showed 2-bromo-5-chloropyridin-3-amine was consumed and one of peak with desired mass was detected. The reaction mixture was filtered, and the filter cake was washed with EtOAc (50 mL×3) and diluted with water (10 mL), extracted with EtOAc (200 mL×3). The combined organics were washed with brine (50 mL), dried over Na₂SO₄, filtered, and concentrated under reduced pressure to give a residue. The residue was dissolved in DCM (80 mL), and 40 g of silica gel was added. The resulting mixture was evaporated under reduced pressure to give a dry flowing solid, and then it was loaded to Biotage® using a 180 g Agela flash silica gel column, eluted with 0% to 40% EtOAc in PE with the flower rate of 75 mL/min. The product fraction was combined and concentrated to give 5-chloro-2-(1-methyl-1H-pyrazol-4-yl)pyridine-3-amine (30 g, 142.35 mmol, 98% yield, 99% purity) as a brown solid.

### Step 2: 5-chloro-3-iodo-2-(1-methyl-1H-pyrazol-4-yl)pyridine

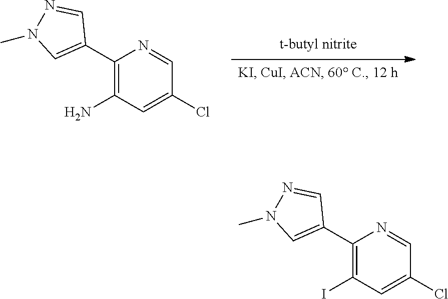

To a solution of 5-chloro-2-(1-methyl-1H-pyrazol-4-yl)pyridine-3-amine (9 g, 43.14 mmol, 1 eq) in MeCN (300 mL) was added CuI (9.86 g, 51.76 mmol, 1.2 eq), KI (35.80 g, 215.68 mmol, 5 eq) and tert-butyl nitrite (22.24 g, 215.68 mmol, 25.65 mL, 5 eq), the mixture was stirred at 60° C. for 12 h under N₂. The reaction was monitored by LCMS, which showed 5-chloro-2-(1-methyl-1H-pyrazol-4-yl)pyridine-3-amine was consumed and one of peak with desired mass was detected. After cooling to rt, the solid was collected by filtration, washed with EtOAc (100 mL×3) and dried under reduced pressure to give a brown solid. The solid was triturated with EtOAc at 20° C. for 30 min. Then the crude product was triturated with ammonia water at 20° C. for 30 min and filtered, the filter cake was concentrated to give 5-chloro-3-iodo-2-(1-methyl-1H-pyrazol-4-yl)pyridine (20.5 g, 50.04 mmol, 58% yield, 78% purity) brown solid.

### Step 3: Methyl(S)-2-((tert-butoxycarbonyl)amino)-3-(5-chloro-2-(1-methyl-1H-pyrazol-4-yl)pyridine-3-yl)propanoate

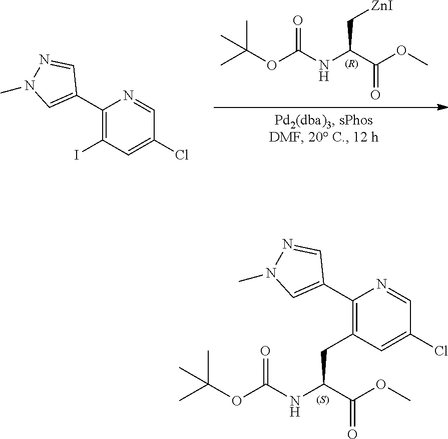

To a solution of 5-chloro-3-iodo-2-(1-methyl-1H-pyrazol-4-yl)pyridine (20.5 g, 64.16 mmol, 1 eq) in DMF (200 mL) was added (R)-(2-((tert-butoxycarbonyl)amino)-3-methoxy-3-oxopropyl)zinc(II) iodide (50.62 g, 128.31 mmol, 2 eq), Pd₂(dba)₃ (2.94 g, 3.21 mmol, 0.05 eq) and sPhos (2.63 g, 6.42 mmol, 0.1 eq), the mixture was stirred at 20° C. for 12 h under N₂. The reaction was monitored by LCMS, which showed 5-chloro-3-iodo-2-(1-methyl-1H-pyrazol-4-yl)pyridine was consumed and one of peak with desired mass was detected. The reaction was quenched by addition of saturated ammonium chloride aqueous solution (200 mL) and extracted with EtOAc (100 mL×3). The combined organics were washed with brine (100 mL×2), dried over Na₂SO₄, filtered, and concentrated under reduced pressure to give a residue. The residue was dissolved in DCM (50 mL), and 40 g of silica gel was added. The resulting mixture was evaporated under reduced pressure to give a dry flowing solid, and then it was loaded to Biotage® using a 120 g Agela flash silica gel column, eluted with 0% to 55% EtOAc in PE with the flower rate of 75 mL/min. The product fraction was combined and concentrated to give methyl(S)-2-((tert-butoxycarbonyl)amino)-3-(5-chloro-2-(1-methyl-1H-pyrazol-4-yl)pyridine-3-yl)propanoate (8 g, 19.86 mmol, 31% yield, 98% purity) as a yellow solid.

### Step 4: (S)-2-((tertbutoxycarbonyl)amino)-3-(5-chloro-2-(1-methyl-1H-pyrazol-4-yl)pyridine-3-yl)propanoic acid

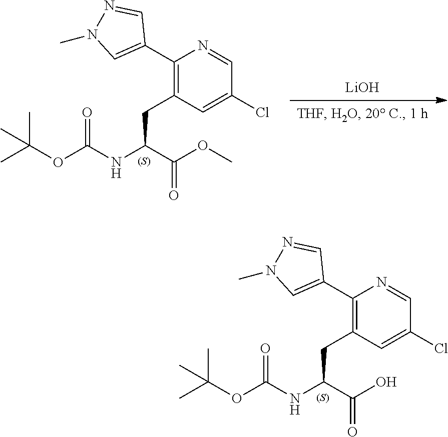

To a solution of methyl(S)-2-((tert-butoxycarbonyl)amino)-3-(5-chloro-2-(1-methyl-1H-pyrazol-4-yl)pyridine-3-yl)propanoate (8 g, 20.26 mmol, 1 eq) in THF (100 mL) was added a solution of LiOH·H₂O (1.02 g, 24.31 mmol, 1.2 eq) in H₂O (50 mL), the mixture was stirred at 20° C. for 1 h. The reaction was monitored by LCMS, which showed methyl(S)-2-((tert-butoxycarbonyl)amino)-3-(5-chloro-2-(1-methyl-1H-pyrazol-4-yl)pyridine-3-yl)propanoate was consumed and one of peak with desired mass was detected. The mixture was extracted with EtOAc (100 mL). The aqueous phase was acidified by added hydrochloric acid (1 M, 50 mL) dropwise at 0° C. to pH=4 and extracted with EtOAc (200 mL×3). The combined organics were washed with brine (50 mL), dried over Na₂SO₄, filtered, and concentrated to give (S)-2-((tertbutoxycarbonyl)amino)-3-(5-chloro-2-(1-methyl-1H-pyrazol-4-yl)pyridine-3-yl)propanoic acid (7 g, 18.01 mmol, 89% yield, 98% purity) as a yellow solid.

### Step 5: Int. 19

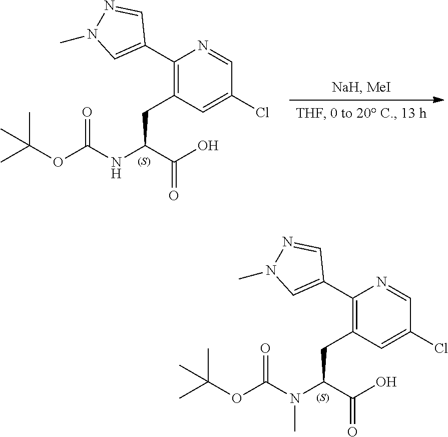

To a solution of (S)-2-((tertbutoxycarbonyl)amino)-3-(5-chloro-2-(1-methyl-1H-pyrazol-4-yl)pyridine-3-yl)propanoic acid (3.7 g, 9.72 mmol, 1 eq) in THF (100 mL) was added NaH (1.94 g, 48.58 mmol, 60% purity, 5 eq) at 0° C., the mixture was stirred at 0° C. for 1 h under N₂. MeI (13.79 g, 97.16 mmol, 6.05 mL, 10 eq) was added to the reaction mixture, the mixture was stirred at 20° C. for 12 h under N₂. The reaction was monitored by LCMS, which showed (S)-2-((tertbutoxycarbonyl)amino)-3-(5-chloro-2-(1-methyl-1H-pyrazol-4-yl)pyridine-3-yl)propanoic acid was consumed and one of peak with desired mass was detected. The reaction mixture was cooled to 0° C. and quenched by NH₄Cl (30 mL), extracted with EtOAc (50 mL×2). The combined organics were washed with brine (30 mL), dried over Na₂SO₄, filtered, and concentrated under reduced pressure to give Int. 19 (3 g, 7.51 mmol, 77% yield, 98% purity) as a yellow solid. ¹H NMR (400 MHz, MeOD-d4) ¹H NMR (400 MHz, METHANOL-d4) δ=8.48-8.32 (m, 1H), 8.08 (d, J=6.8 Hz, 1H), 7.92 (s, 1H), 7.76-7.64 (m, 1H), 4.80-4.66 (m, 1H), 3.98 (s, 3H), 3.64-3.56 (m, 1H), 3.28-3.16 (m, 1H), 2.66 (d, J=9.8 Hz, 3H), 1.24 (s, 9H)

### Int. 20: Preparation of (S)-2-((tertbutoxycarbonyl)(methyl)amino)-3-(5-chloro-2-(1-methylcyclopropoxy)phenyl)propanoic acid

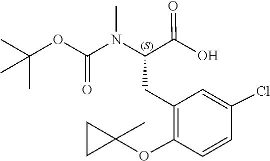

### Step 1: 4-chloro-1-(1-methylcyclopropoxy)-2-nitrobenzene

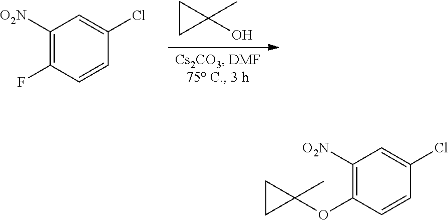

To a solution of 4-chloro-1-fluoro-2-nitrobenzene (21 g, 119.63 mmol, 1 eq) and 1-methylcyclopropanol (11.16 g, 131.59 mmol, 85% purity, 1.1 eq) in DMF (150 mL) was added Cs₂CO₃ (58.47 g, 179.44 mmol, 1.5 eq). Then, the reaction mixture was stirred at 75° C. for 3 h. HPLC showed starting material was consumed completely and a new major peak was detected. The reaction mixture was cooled to rt and quenched by water (200 mL), extracted with EtOAc (150 mL×2). The combined organics were washed with water (100 mL×3) and brine (150 mL), dried over Na₂SO₄, filtered, and concentrated under reduced pressure to give 4-chloro-1-(1-methylcyclopropoxy)-2-nitrobenzene (25 g, 104.54 mmol, 87% yield, 95.2% purity) as a brown oil.

### Step 2: 5-chloro-2-(1-methylcyclopropoxy)aniline

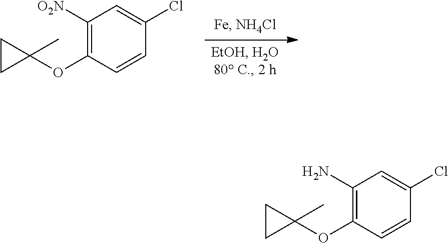

To a solution of 4-chloro-1-(1-methylcyclopropoxy)-2-nitrobenzene (25 g, 109.82 mmol, 1 eq) in EtOH (200 mL) and H₂O (100 mL) was added NH₄Cl (5.87 g, 109.82 mmol, 1 eq) and Fe (18.40 g, 329.46 mmol, 3 eq). Then, the reaction mixture was stirred at 80° C. for 2 h. LCMS showed starting material was consumed completely and desired mass was detected. The resultant mixture was filtered, and the filter cake was rinsed with EtOH (100 mL×3). Then the combined filtrates were concentrated under reduced pressure to remove EtOH. Then the aqueous phase was extracted with EtOAc (150 mL×2). The combined organics were washed with brine (100 mL), dried over Na₂SO₄, filtered, and concentrated under reduced pressure to give 5-chloro-2-(1-methylcyclopropoxy)aniline (20.9 g, 104.54 mmol, 95% yield, 99% purity) as a black, brown oil.

### Step 3: 4-chloro-2-iodo-1-(1-methylcyclopropoxy)benzene

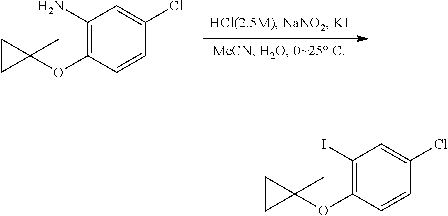

To a solution of 5-chloro-2-(1-methylcyclopropoxy)aniline (20.9 g, 105.74 mmol, 1 eq) in HCl (2.5 M, 209.00 mL, 4.94 eq) and ACN (100 mL) was added a solution of NaNO₂ (8.02 g, 116.31 mmol, 1.1 eq) in H₂O (100 mL) slowly at 0° C. Then, a solution of KI (43.88 g, 264.34 mmol, 2.5 eq) in H₂O (100 mL) was added slowly. Then the reaction mixture was allowed to warm to 25° C. and stirred at 25° C. for 12 h. LCMS showed starting material was consumed completely and a new major spot was detected. The reaction mixture was adjusted pH=7 by adding saturated NaHCO₃ aqueous solution slowly at 0° C., extracted with EtOAc (60 mL×2). The combined organics were washed with brine (80 mL), dried over Na₂SO₄, filtered, and concentrated under reduced pressure to give a residue. The residue was purified by flash silica gel chromatography (ISCO®; 220 g SepaFlash® Silica Flash Column, Eluent of 0˜0% EtOAc/PE gradient @ 150 mL/min) to give 4-chloro-2-iodo-1-(1-methylcyclopropoxy)benzene (23 g, 73.84 mmol, 70% yield, 99.1% purity) as a light-yellow oil.

### Step 4: Methyl(S)-2-((tert-butoxycarbonylamino)-3-(5-chloro-2-(1-methylcyclopropoxy)phenyl)propanoate

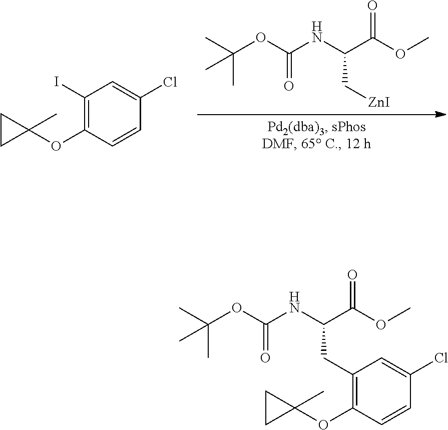

A mixture of 4-chloro-2-iodo-1-(1-methylcyclopropoxy)benzene (15 g, 48.62 mmol, 1 eq), sPhos (2.00 g, 4.86 mmol, 0.1 eq) and Pd₂(dba)₃ (2.23 g, 2.43 mmol, 0.05 eq) in DMF (50 mL) was degassed and purged with N₂ 3 times, then [(2R)-2-(tert-butoxycarbonylamino)-3-methoxy-3-oxo-propyl]-iodo-zinc (41.95 g, 106.34 mmol, 2.19 eq) was added to the mixture and then the mixture was stirred at 65° C. for 12 h under N₂ atmosphere. LCMS showed starting material was consumed completely and desired mass was detected. The reaction mixture was cooled to rt and quenched by water (300 mL), extracted with EtOAc (150 mL×2). The combined organics were washed with water (200 mL×2) and brine (200 mL), dried over Na₂SO₄, filtered, and concentrated under reduced pressure to give a residue. The residue was purified by prep-HPLC (column: Welch Xtimate C18 250*70 mm #10 um; mobile phase: [water (NH₄HCO₃)-ACN]; B %: 50%-80%, 20 min) to give methyl(S)-2-((tert-butoxycarbonylamino)-3-(5-chloro-2-(1-methylcyclopropoxy)phenyl)propanoate (15 g, 36.75 mmol, 76% yield, 94.0% purity) as a light-yellow solid.

### Step 5: (S)-2-((tert-butoxycarbonyl)amino)-3-(5-chloro-2-(1-methylcyclopropoxy)phenyl) propanoic acid

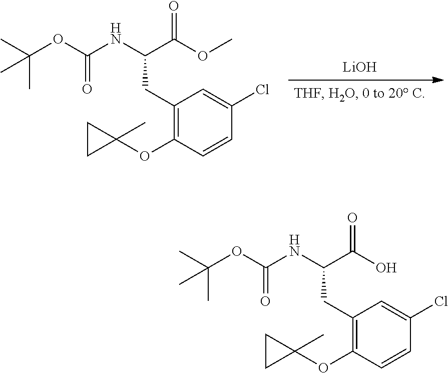 [SMILES: COC(=O)[C@H](Cc1cc(Cl)ccc1OC1(C)CC1)NC(=O)OC(C)(C)C ; "LiOH" ; "THF, H2O, 0 to 20° C." ; CC(C)(C)OC(=O)N[C@@H](Cc1cc(Cl)ccc1OC1(C)CC1)C(=O)O]

To a solution of methyl(S)-2-((tert-butoxycarbonylamino)-3-(5-chloro-2-(1-methylcyclopropoxy)phenyl)propanoate (15 g, 39.08 mmol, 1 eq) in THF (200 mL) was added a solution of LiOH·H₂O (2.46 g, 58.61 mmol, 1.5 eq) in H₂O (100 mL) at 0-10° C. Then the reaction mixture was stirred at 20° C. for 0.5 hr. LCMS showed starting material was consumed completely and desired mass was detected. The reaction mixture was concentrated under reduced pressure to remove THF and extracted with EtOAc (80 mL×2). The aqueous phase was acidified by adding hydrochloric acid (1N) dropwise at 0° C. to pH=2-3. The aqueous phase was extracted with EtOAc (100 mL×2). The combined organics were washed with brine (150 mL), dried over Na₂SO₄, filtered and concentrated under reduced pressure to give (S)-2-((tert-butoxycarbonyl)amino)-3-(5-chloro-2-(1-methylcyclopropoxy)phenyl) propanoic acid (12.7 g, 33.65 mmol, 86% yield, 98.1% purity) as a light-yellow oil.

### Step 6: Int. 20

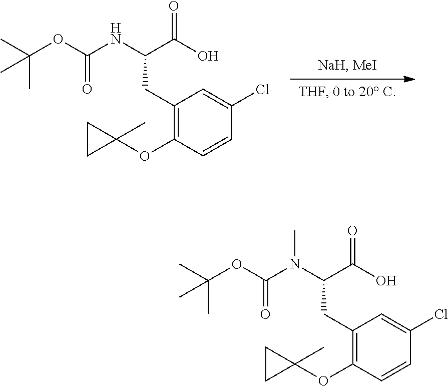

To a solution of (S)-2-((tert-butoxycarbonyl)amino)-3-(5-chloro-2-(1-methylcyclopropoxy)phenyl) propanoic acid (12.7 g, 34.34 mmol, 1 eq) in THF (400 mL) was added NaH (6.87 g, 171.70 mmol, 60% purity, 5 eq) at 0° C. under N₂ atmosphere. The mixture was stirred at 20° C. for 0.5 hr. Then MeI (48.74 g, 343.39 mmol, 21.38 mL, 10 eq) was added and the reaction mixture was stirred at 20° C. for 12 h. LCMS showed starting material was consumed completely and desired mass was detected. The reaction mixture was cooled to 0° C. and quenched by NH₄Cl (150 mL), acidified by adding hydrochloric acid (1 N) dropwise at 0° C. to pH=2-3 and extracted with EtOAc (200 mL×2). The combined organics were washed with brine (200 mL), dried over Na₂SO₄, filtered, and concentrated under reduced pressure to give Int. 20 (9.98 g, 26.00 mmol, 76% yield, 100% purity) as a light-yellow solid. ¹H NMR (400 MHz, DMSO-d₆) δ=7.28-7.04 (m, 3H), 4.83-4.39 (m, 1H), 3.15-2.97 (m, 1H), 2.89-2.71 (m, 1H), 2.61 (br d, J=3.3 Hz, 3H), 1.49 (d, J=11.1 Hz, 3H), 1.30-1.12 (m, 9H), 0.95-0.81 (m, 2H), 0.81-0.69 (m, 2H)

### Int. 22: Preparation of (S)-3-(1′-acetyl-5-chloro-1′,2′,3′,6′-tetrahydro-[2,4′-bipyridin]-3-yl)-2-((tert-butoxycarbonyl)(methyl)amino)propanoic acid

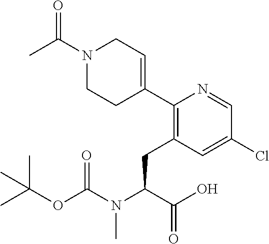 [SMILES: CC(=O)N1CC=C(c2ncc(Cl)cc2C[C@@H](C(=O)O)N(C)C(=O)OC(C)(C)C)CC1]

### Step 1: 2-bromo-3-(bromomethyl)-5-chloropyridine

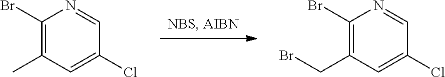

To a stirred mixture of 2-bromo-5-chloro-3-methylpyridine (25 g, 121.08 mmol, 1 eq.) and NBS (23706.05 mg, 133.19 mmol, 1.1 eq) in ACN (200 mL) was added AIBN (1988.33 mg, 12.11 mmol, 0.1 eq.) at rt under nitrogen atmosphere. The resulting mixture was stirred overnight at 80° C. The resulting mixture was concentrated in vacuo and diluted with water. The mixture was extracted with EtOAc (2×200 mL) and the combined organic layers were dried over anhydrous Na₂SO₄ and filtered. The filtrate was concentrated under vacuum. This resulted in 2-bromo-3-(bromomethyl)-5-chloropyridine (30 g, 87%) as a yellow oil. The crude product was used in the next step directly without further purification. LCMS (ESI+): m/z 282.4.

### Step 2: Tert-butyl (2S)-3-(2-bromo-5-chloropyridin-3-yl)-2-[(diphenylmethylidene)amino]propanoate

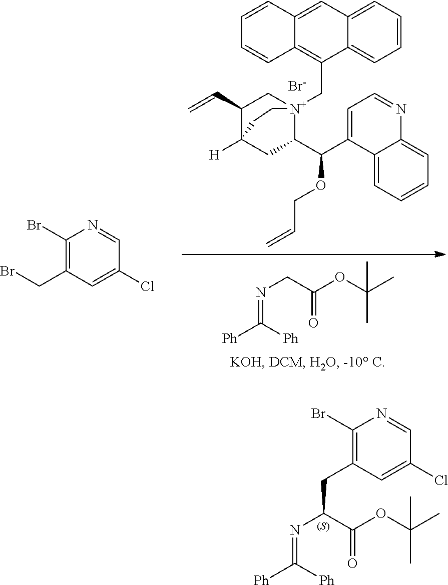

To a stirred mixture of 2-bromo-3-(bromomethyl)-5-chloropyridine (15 g, 52.57 mmol, 1 eq.), (2S,4S,5R)-1-[(anthracen-9-yl)methyl]-5-ethenyl-2-[(R)-(prop-2-en-1-yloxy)(quinolin-4-yl)methyl]-1-azabicyclo[2.2.2]octan-1-ium bromide (1591.69 mg, 2.63 mmol, 0.05 eq.) and isopropyl 2-[(diphenylmethylidene)amino]acetate (29578.95 mg, 105.13 mmol, 2 eq.) in DCM (100 mL) was added KOH (29.49 g, 525.65 mmol, 10 eq.) in H₂O (30 mL) in portions at −10° C. under air atmosphere. The resulting mixture was stirred for an additional 4 h at −10° C. The resulting mixture was extracted with EtOAc (2×200 mL), and the combined organic layers were dried over anhydrous Na₂SO₄ and filtered. The filtrate was concentrated under vacuum. The residue was purified by silica gel column chromatography, eluted with PE/EA (10:1) to afford tert-butyl (2S)-3-(2-bromo-5-chloropyridin-3-yl)-2-[(diphenylmethylidene)amino]propanoate (25 g, 95%) as a yellow oil. LCMS (ESI+): m/z 501.1.

### Step 3: (S)-2-amino-3-(2-bromo-5-chloropyridin-3-yl)propanoic acid

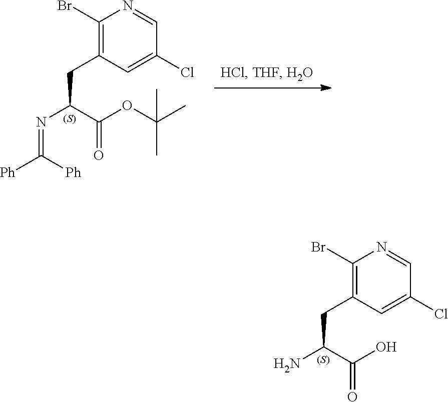

A mixture of tert-butyl (2S)-3-(2-bromo-5-chloropyridin-3-yl)-2-[(diphenylmethylidene)amino]propanoate (5 g, 10.0 mmol, 1 eq.) and HCl (6M) (30 mL) in THF (10 mL) was stirred overnight at 55° C. The resulting mixture was concentrated under vacuum. This resulted in (2S)-2-amino-3-(2-bromo-5-chloropyridin-3-yl)propanoic acid (2.6 g, 93%) as a yellow oil. The crude product was used in the next step directly without further purification. LCMS (ESI+): m/z 278.9.

### Step 4: (2S)-3-(2-bromo-5-chloropyridin-3-yl)-2-[(tert-butoxycarbonyl)amino]propanoic acid

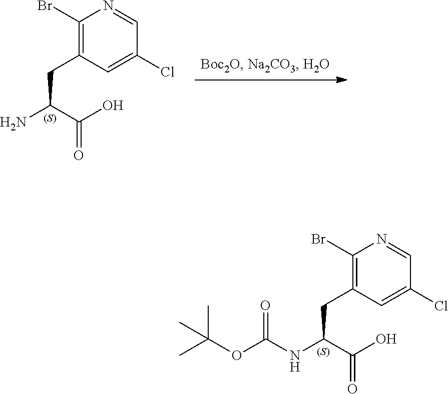

To a stirred mixture of (2S)-2-amino-3-(2-bromo-5-chloropyridin-3-yl)propanoic acid (9 g, 32.20 mmol, 1 eq.) and Na₂CO₃ (10.24 g, 96.59 mmol, 3 eq.) in H₂O (60 mL) was added Boc₂O (14.05 g, 64.40 mmol, 2 eq.) in portions at rt under air atmosphere. The resulting mixture was stirred overnight at rt. The mixture was acidified to pH=6 with diluted HCl (aq. 1N). The resulting mixture was extracted with EtOAc (2×100 mL), and the combined organic layers were dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% FA), 10% to 100% gradient in 30 min; detector, UV 210 nm. This resulted in (2S)-3-(2-bromo-5-chloropyridin-3-yl)-2-[(tert-butoxycarbonyl)amino]propanoic acid (12 g, 98%) as a white solid. LCMS (ESI+): m/z 380.90.

### Step 5: (2S)-3-(2-bromo-5-chloropyridin-3-yl)-2-[(tert-butoxycarbonyl)(methyl)amino]propanoic acid

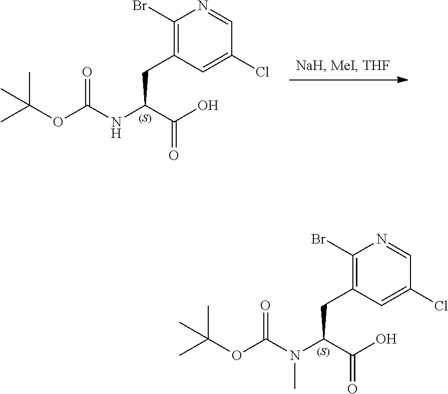

NaH (6.32 g, 263.41 mmol, 10 eq.) was added into a stirred mixture of (2S)-3-(2-bromo-5-chloropyridin-3-yl)-2-[(tert-butoxycarbonyl)amino]propanoic acid (10 g, 26.34 mmol, 1 eq.) in THF (100 mL) portion wise at 0° C. under nitrogen and the resulting mixture was stirred for 1 h at 0° C. MeI (56.08 g, 395.12 mmol, 15 eq.) was added dropwise at 0° C. under nitrogen atmosphere. The resulting mixture was stirred overnight at rt. The reaction was quenched with water at 0° C. The mixture was acidified to pH=6 with HCl (aq.). The resulting mixture was extracted with EtOAc (2×120 mL) and dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 100% gradient in 30 min; detector, UV 220 nm. This resulted in (2S)-3-(2-bromo-5-chloropyridin-3-yl)-2-[(tert-butoxycarbonyl)(methyl)amino]propanoic acid (7.3 g, 70%) as a yellow oil.

### Step 6: Int. 22

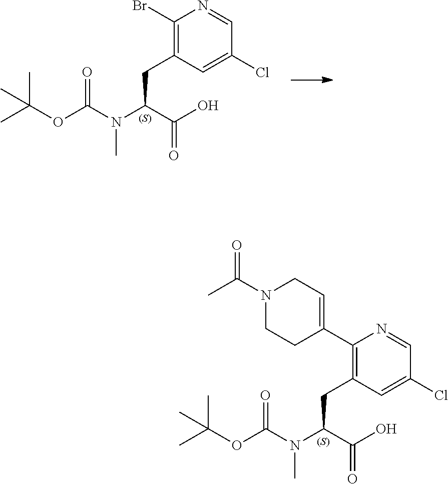

To a stirred mixture of (2S)-3-(2-bromo-5-chloropyridin-3-yl)-2-[(tert-butoxycarbonyl)(methyl)amino]propanoic acid (2.5 g, 6.35 mmol, 1 eq.) and 1-[4-(4,4,5,5-tetramethyl-1,3,2-dioxaborolan-2-yl)-3,6-dihydro-2H-pyridin-1-yl]ethanone (2392.26 mg, 9.53 mmol, 1.5 eq.) in dioxane (20 mL) was added Na₂CO₃ (2019.28 mg, 19.05 mmol, 3 eq.) and Pd(dppf)Cl₂·DCM Adduct (517.34 mg, 0.635 mmol, 0.1 eq) in portions at rt under nitrogen atmosphere. The resulting mixture was stirred for an additional 3 h at 80° C. The mixture was acidified to pH=6 with HCl (aq. 1N). The resulting mixture was extracted with EtOAc (2×60 mL), and the combined organic layers were dried over anhydrous Na₂SO₄ and filtered. The filtrate was concentrated under vacuum. The residue was purified by reversed-phase flash chromatography with the following conditions: column, C18 silica gel; mobile phase, MeCN in Water (0.1% FA), 10% to 100% gradient in 30 min; detector, UV 254 nm. This resulted in Int. 22 (1.94 g, 66%) as a brown solid. LCMS (ESI+): m/z 438.2. ¹H NMR (400 MHz, DMSO-d₆) δ 12.99 (s, 1H), 8.45 (dd, J=15.5, 2.4 Hz, 1H), 7.81 (dd, J=53.7, 2.4 Hz, 1H), 5.86 (d, J=33.1 Hz, 1H), 4.85-4.50 (m, 1H), 4.09 (tt, J=30.1, 13.7 Hz, 2H), 3.90-3.47 (m, 2H), 3.42-3.24 (m, 2H), 3.11 (dt, J=26.2, 13.1 Hz, 1H), 2.58-2.37 (m, 4H), 2.41-2.15 (m, 1H), 2.05 (dd, J=12.3, 2.5 Hz, 3H), 1.23 (dd, J=31.0, 3.0 Hz, 9H).

### Int. 23: Preparation of (S)-2-((tert-butoxycarbonyl)(methyl)amino)-3-(5-chloro-2-cyclopropoxyphenyl)propanoic acid

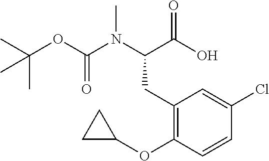 [SMILES: CN(C(=O)OC(C)(C)C)[C@@H](Cc1cc(Cl)ccc1OC1CC1)C(=O)O]

### Step 1: 4-chloro-1-cyclopropoxy-2-nitrobenzene

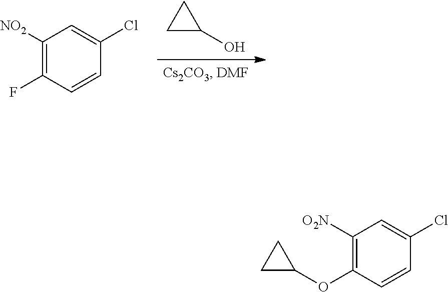

To a solution of 4-chloro-1-fluoro-2-nitrobenzene (40 g, 227.86 mmol, 1 eq) in DMF (400 mL) was added cyclopropanol (19.85 g, 341.79 mmol, 1.5 eq) and Cs₂CO₃ (111.36 g, 341.79 mmol, 1.5 eq), the mixture was stirred at 75° C. for 12 h under N₂. The reaction was monitored by LCMS, which showed 4-chloro-1-fluoro-2-nitrobenzene was consumed and one of peak with desired mass was detected. After cooling to rt, the filtrate was diluted with water (400 mL), extracted with EtOAc (200 mL×3). The combined organics were washed with brine (400 mL), dried over Na₂SO₄, filtered, and concentrated under reduced pressure to give a residue. 4-chloro-1-cyclopropoxy-2-nitrobenzene (35 g, crude) was obtained as a black, brown oil.

### Step 2: 5-chloro-2-cyclopropoxyaniline

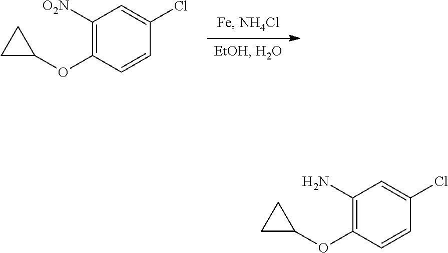

To a solution of 4-chloro-1-cyclopropoxy-2-nitrobenzene (35 g, 163.84 mmol, 1 eq) in H₂O (280 mL) and ethanol (280 mL) was added Fe (45.75 g, 819.22 mmol, 5 eq) and NH₄Cl (4.38 g, 81.92 mmol, 0.5 eq), the mixture was stirred at 80° C. for 1 h. The reaction was monitored by LCMS, which showed 4-chloro-1-cyclopropoxy-2-nitrobenzene was consumed and one of peak with desired mass was detected. After cooling to rt, the reaction mixture was filtered, and the filter cake was rinsed with EtOAc (100 mL×3). Then the combined filtrates were concentrated under reduced pressure to give a residue, 5-chloro-2-cyclopropoxyaniline (26 g, 115.93 mmol, 71% yield, 82% purity) was obtained as a yellow oil.

### Step 3: 4-chloro-1-cyclopropoxy-2-iodobenzene

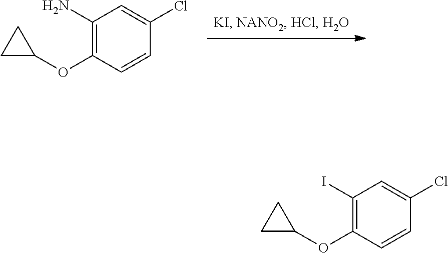

To a solution of 5-chloro-2-cyclopropoxyaniline (20 g, 108.91 mmol, 1 eq) in H₂O (216 mL) was added hydrogen chloride (12 M, 27.23 mL, 3 eq) dropwise at 0° C. adjusted pH to 2, the mixture was stirred at 0° C. for 0.5 hr, a solution of NaNO₂ (8.27 g, 119.80 mmol, 1.1 eq) in H₂O (36 mL) was added to the mixture dropwise at 0° C. and stirred for 1 hr. Then the reaction mixture was added to a solution of KI (54.24 g, 326.74 mmol, 3 eq) in H₂O (200 mL), the mixture was stirred at 20° C. for 12 hr. The reaction was monitored by LCMS, which showed 5-chloro-2-cyclopropoxyaniline was consumed and one of peak with desired mass was detected. The mixture was filtered, and filtrate was diluted with water (300 mL), extracted with EtOAc (150 mL×3). The combined organics were washed with brine (150 mL), dried over Na₂SO₄, filtered, and concentrated under reduced pressure to give residue. The residue was dissolved in DCM (100 mL), and 60 g of silica gel was added. The resulting mixture was evaporated under reduced pressure to give a dry flowing solid, and then it was loaded to Biotage® using a 120 g agela flash silica gel column, eluted with 0% to 12% EtOAc in PE with the flower rate of 75 mL/min. The product fraction was combined and evaporated. 4-chloro-1-cyclopropoxy-2-iodobenzene (20 g, 65.46 mmol, 60% yield, 96.4% purity) was obtained as a black, brown oil.

### Step 4: Methyl(S)-2-((tert-butoxycarbonyl)amino)-3-(5-chloro-2-cyclopropoxyphenyl)propanoate

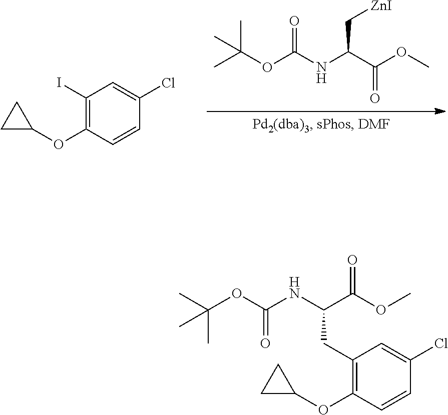

A mixture of Zn (34.77 g, 531.70 mmol, 3.5 eq) in DMF (210 mL) was degassed and purged with N₂ for 3 times, and the mixture was stirred at 120° C. for 10 min under N₂ atmosphere. Then 12 (11.57 g, 45.57 mmol, 9.18 mL, 0.3 eq) in DMF (120 mL) was added dropwise at 20° C., methyl (R)-2-((tert-butoxycarbonyl)amino)-3-iodopropanoate (50 g, 151.92 mmol, 1 eq) in DMF (40 mL) was added dropwise, I₂ (11.57 g, 45.57 mmol, 9.18 mL, 0.3 eq) in DMF (40 mL) was added dropwise, the reaction mixture was stirred at 20° C. for 1 hr. The reaction was monitored by TLC (PE:EtOAc=5:1, Rf=0.5) showed starting material was consumed completely and a new major spot was detected. The crude product was used into the next step without further purification. (R)-(2-((tert-butoxycarbonyl)amino)-3-methoxy-3-oxopropyl)zinc(II) iodide (59.93 g, crude) was obtained as a grey liquid. The zincate was used without further purification.

To a solution of 4-chloro-1-cyclopropoxy-2-iodobenzene (20 g, 67.91 mmol, 1 eq), sPhos (4.18 g, 10.19 mmol, 0.15 eq) and Pd₂(dba)₃ (6.22 g, 6.79 mmol, 0.1 eq) in DMF (400 mL) was degassed and purged with N₂ for 3 times. Then (R)-(2-((tert-butoxycarbonyl)amino)-3-methoxy-3-oxopropyl)zinc(II) iodide (53.58 g, 135.82 mmol, 2 eq) in DMF was added dropwise. Then the reaction mixture was stirred at 20° C. for 12 hr. The reaction was monitored by LCMS, which showed 4-chloro-1-cyclopropoxy-2-iodobenzene was consumed and one of peaks with desired mass was detected. The reaction was quenched by addition of saturated ammonium chloride aqueous solution (200 mL) and filtered, the filtrate was diluted with water (100 mL), extracted with EtOAc (100 mL×3). The combined organics were washed with brine (50 mL), dried over Na₂SO₄, filtered, and concentrated under reduced pressure to give a residue. The residue was purified by preparative HPLC (column: Waters Xbridge BEH C18 250×70 mm×10 um; mobile phase: [water (NH₄HCO₃)-ACN]; B %:50%-80%, 18 min) the HPLC fractions were combined, lyophilized. Methyl(S)-2-((tert-butoxycarbonyl)amino)-3-(5-chloro-2-cyclopropoxyphenyl)propanoate (8 g, 19.53 mmol, 29% yield, 98% purity) was obtained as a yellow oil.

### Step 5: (S)-2-((tert-butoxycarbonyl)amino)-3-(5-chloro-2-cyclopropoxyphenyl)propanoic acid

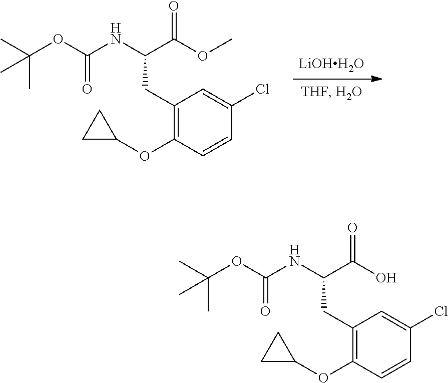

To a solution of methyl(S)-2-((tert-butoxycarbonyl)amino)-3-(5-chloro-2-cyclopropoxyphenyl)propanoate (8 g, 21.63 mmol, 1 eq) in THF (112 mL) was added LiOH·H₂O (1.09 g, 25.96 mmol, 1.2 eq) in H₂O (56 mL), the mixture was stirred at 20° C. for 1 hr. The reaction was monitored by LCMS, which showed methyl(S)-2-((tert-butoxycarbonyl)amino)-3-(5-chloro-2-cyclopropoxyphenyl)propanoate was consumed and one of peak with desired mass was detected. The mixture was acidified pH=5 by adding hydrochloric acid (0.1 M, 15 mL) dropwise at 0° C. The residue was diluted with water (100 mL), extracted with EtOAc (40 mL×3). The combined organics were washed with brine (50 mL), dried over Na₂SO₄, filtered, and concentrated under reduced pressure. (S)-2-((tert-butoxycarbonyl)amino)-3-(5-chloro-2-cyclopropoxyphenyl)propanoic acid (5 g, 13.77 mmol, 64% yield, 98% purity) was obtained as a yellow solid.

### Step 6: Int. 23

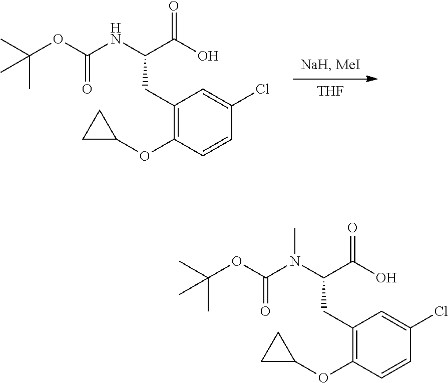

To a solution of (S)-2-((tert-butoxycarbonyl)amino)-3-(5-chloro-2-cyclopropoxyphenyl)propanoic acid (5 g, 14.05 mmol, 1 eq) in THF (50 mL) was added NaH (2.81 g, 70.26 mmol, 60% purity, 5 eq) at 0° C., the mixture was stirred at 0° C. for 1 h under N₂, MeI (19.95 g, 140.52 mmol, 8.75 mL, 10 eq) was added to the reaction mixture, the mixture was stirred at 20° C. for 12 h. The reaction was monitored by LCMS, which showed (S)-2-((tert-butoxycarbonyl)amino)-3-(5-chloro-2-cyclopropoxyphenyl)propanoic acid was consumed and one of peak with desired mass was detected. The reaction was quenched by addition of saturated ammonium chloride aqueous solution (20 mL), the mixture was acidified pH=5 by adding hydrochloric acid (0.1 M, 10 mL) dropwise at 0° C. The residue was extracted with EtOAc (20 mL×3). The combined organics were washed with brine (20 mL), dried over Na₂SO₄, filtered, and concentrated under reduced pressure to give a residue Int. 23 (3 g, 7.71 mmol, 54% yield, 95% purity) was obtained as a yellow oil. ¹H NMR (400 MHz, DMSO-d6) ¹H NMR (400 MHz, DMSO-d6) δ=7.31-7.08 (m, 3H), 4.85-4.52 (m, 1H), 3.91-3.80 (m, 1H), 3.12-2.99 (m, 1H), 2.94-2.80 (m, 1H), 2.65-2.55 (m, 3H), 1.24 (br d, J=19.6 Hz, 9H), 0.87-0.58 (m, 4H)

### Int. 24: Preparation of (2S)-2-[(tert-butoxycarbonyl)(methyl)amino]-3-[5-chloro-2-(morpholin-4-yl)phenyl]propanoic acid

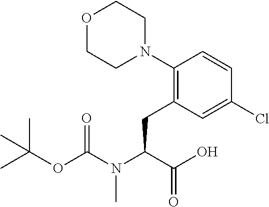 [SMILES: CN(C(=O)OC(C)(C)C)[C@@H](Cc1cc(Cl)ccc1N1CCOCC1)C(=O)O]

### Step 1: (2S)-2-[(tert-butoxycarbonyl)(methyl)amino]-3-[5-chloro-2-(morpholin-4-yl)phenyl]propanoate

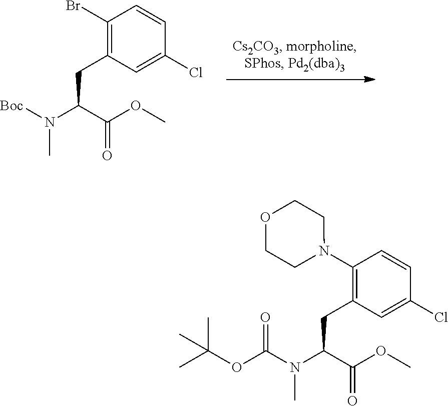 [SMILES: COC(=O)[C@H](Cc1cc(Cl)ccc1Br)N(C)C(=O)OC(C)(C)C ; "Cs2CO3, morpholine,SPhos, Pd2(dba)3" ; COC(=O)[C@H](Cc1cc(Cl)ccc1N1CCOCC1)N(C)C(=O)OC(C)(C)C]

A solution of methyl (2S)-3-(2-bromo-5-chlorophenyl)-2-[(tert-butoxycarbonyl)(methyl)amino]propanoate (3 g, 7.376 mmol, 1 equiv.), Cs2CO3 (7232.46 mg, 22.128 mmol, 3 equiv.), morpholine (771.18 mg, 8.851 mmol, 1.2 equiv.), SPhos (605.66 mg, 1.475 mmol, 0.2 equiv.) and Pd2(dba)3 (675.48 mg, 0.738 mmol, 0.1 equiv.) in dioxane (50 mL) was stirred for 3 h at 80° C. under nitrogen atmosphere. The residual Cs2CO3 was removed by filtration. The residue was purified by silica gel column chromatography, eluted with PE/EA (5:1) to afford methyl (2S)-2-[(tert-butoxycarbonyl)(methyl)amino]-3-[5-chloro-2-(morpholin-4-yl)phenyl]propanoate (1.15 g, 38%) as a brown oil. LCMS (ESI+): m/z 413 (M+H⁺).

### Step 2: Int. 24

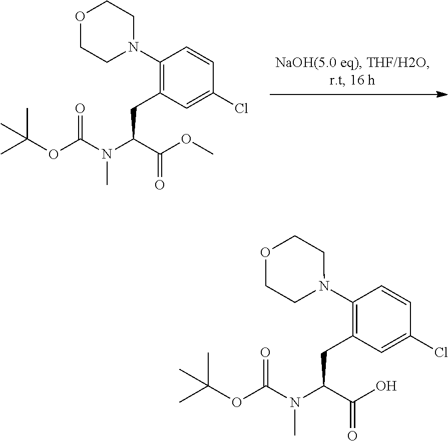 [SMILES: COC(=O)[C@H](Cc1cc(Cl)ccc1N1CCOCC1)N(C)C(=O)OC(C)(C)C ; "NaOH(5.0 eq), THF/H2O,r.t, 16 h" ; CN(C(=O)OC(C)(C)C)[C@@H](Cc1cc(Cl)ccc1N1CCOCC1)C(=O)O]

To a stirred solution of methyl (2S)-2-[(tert-butoxycarbonyl)(methyl)amino]-3-[5-chloro-2-(morpholin-4-yl)phenyl]propanoate (1.2 g, 2.906 mmol, 1 equiv.) in tetrahydrofuran (15 mL) was added caustic soda (0.58 g, 14.530 mmol, 5 equiv.) in water (5 mL) dropwise at 0° C. The resulting mixture was stirred for 16 h at rt. The resulting mixture was concentrated under reduced pressure and then diluted with water (20 mL). The resulting mixture was acidified to pH=3 with diluted citric acid (1N) and then extracted with EtOAc (3×100 mL). The combined organic layers were washed with brine (1×100 mL) and dried over anhydrous Na2SO₄. The residue was purified by reversed-phase flash chromatography with the following conditions: column, C18 silica gel; mobile phase, MeCN in Water (0.1% FA), 10% to 50% gradient in 10 min; detector, UV 220 nm. After filtration, the filtrate was concentrated under reduced pressure. This resulted in Int. 24 (0.81 g, 70%) as a Brown yellow solid. LCMS (ESI+): m/z 399 (M+H⁺).

### Int. 25: Preparation of (S)-2-((tert-butoxycarbonyl)(methyl)amino)-3-(5-chloro-2-morpholinopyridin-3-yl)propanoic acid

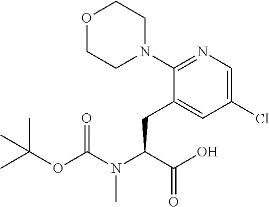 [SMILES: CN(C(=O)OC(C)(C)C)[C@@H](Cc1cc(Cl)cnc1N1CCOCC1)C(=O)O]

### Step 1: 4-(3-bromo-5-chloropyridin-2-yl)morpholine

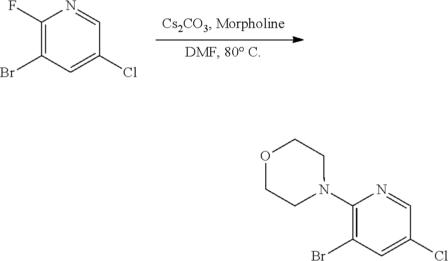 [SMILES: Fc1ncc(Cl)cc1Br ; "Cs2CO3, Morpholine" ; "DMF, 80° C." ; Clc1cnc(N2CCOCC2)c(Br)c1]

A solution of 3-bromo-5-chloro-2-fluoropyridine (10 g, 47.52 mmol, 1 eq) in DMF (600 mL) was treated with Cs₂CO₃ (46.45 g, 142.57 mmol, 3 eq) at rt under nitrogen atmosphere followed by the addition of morpholine (6.21 g, 71.28 mmol, 1.5 eq.) in portions at rt. The resulting mixture was stirred for 16 h at rt under nitrogen atmosphere. Desired product could be detected by LCMS. The resulting mixture was diluted with ice water (1 L) and extracted with EtOAc (2×1 L). The combined organic layers were washed with brine (500 mL×3) and dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 100% gradient in 40 min; detector, UV 200.210 nm. This resulted in 4-(3-bromo-5-chloropyridin-2-yl)morpholine (3 g, 23%) as a light-yellow solid. LCMS (ESI+): m/z 279.00.

### Step 2: Methyl (2S)-2-[(tert-butoxycarbonyl)amino]-3-[5-chloro-2-(morpholin-4-yl)pyridin-3-yl]propanoate

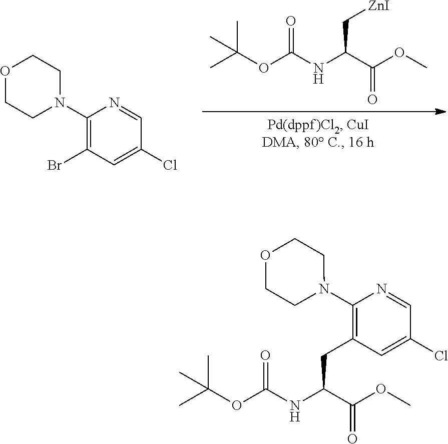

A solution of 4-(3-bromo-5-chloropyridin-2-yl)morpholine (3.3 g, 11.89 mmol, 1 eq.) in DMA (30 mL) was treated with CuI (0.45 g, 2.38 mmol, 0.2 eq.) and Pd(dppf)Cl₂·CH₂Cl₂ (0.87 g, 1.19 mmol. 0.1 eq.) at rt under nitrogen atmosphere followed by the addition of methyl (2R)-2-[(tert-butoxycarbonyl)amino]-3-(iodozincio)propanoate (33 mL) at rt. The resulting mixture was stirred for 16 h at 80° C. under nitrogen atmosphere. Desired product could be detected by LCMS. The reaction was quenched with water (200 mL) and diluted with EA (200 mL). The precipitated solids were removed by filtration and washed with EtOAc (2×200 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 100% gradient in 40 min; detector, UV 200.210 nm. The residue was purified by Prep-TLC (PE/EA 2:1) to afford methyl (2S)-2-[(tert-butoxycarbonyl)amino]-3-[5-chloro-2-(morpholin-4-yl)pyridin-3-yl]propanoate (1 g, 21%) as a light-yellow oil. LCMS (ESI+): m/z 400.15.

### Step 3: Methyl (2S)-2-[(tert-butoxycarbonyl)(methyl)amino]-3-[5-chloro-2-(morpholin-4-yl)pyridin-3-yl]propanoate

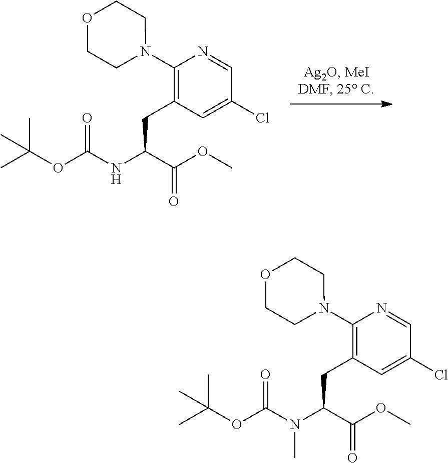 [SMILES: COC(=O)[C@H](Cc1cc(Cl)cnc1N1CCOCC1)NC(=O)OC(C)(C)C ; "Ag2O, MeIDMF, 25° C." ; COC(=O)[C@H](Cc1cc(Cl)cnc1N1CCOCC1)N(C)C(=O)OC(C)(C)C]

A solution of methyl (2S)-2-[(tert-butoxycarbonyl)amino]-3-[5-chloro-2-(morpholin-4-yl)pyridin-3-yl]propanoate (700 mg, 1.75 mmol, 1 eq.) in DMF (10 mL, 129.22 mmol) was treated with Ag₂O (2 g, 8.76 mmol, 5 eq.) at rt under nitrogen atmosphere followed by the addition of CH₃I (2.4 g, 17.51 mmol, 10 eq.) at rt. The resulting mixture was stirred for 16 h at rt under nitrogen atmosphere. Desired product could be detected by LCMS. The resulting mixture was extracted with EtOAc (2×100 mL). The combined organic layers were dried over anhydrous Na₂SO₄. After filtration, the filtrate was concentrated under reduced pressure. The residue was purified by Prep-TLC (PE/EA 2:1) to afford methyl (2S)-2-[(tert-butoxycarbonyl)(methyl)amino]-3-[5-chloro-2-(morpholin-4-yl)pyridin-3-yl]propanoate (500 mg, 69%) as a light-yellow oil. LCMS (ESI+): m/z 414.15. ¹H NMR (400 MHz, DMSO-d₆): δ1.15-1.34 (m, 8H), 2.61 (d, J=8.9 Hz, 2H), 2.94-3.13 (m, 4H), 3.23 (dd, J=14.7, 4.5 Hz, 1H), 3.66-3.75 (m, 5H), 7.69 (dd, J=34.4, 2.6 Hz, 1H), 8.22 (dd, J=13.9, 2.6 Hz, 1H).

### Step 4: Int. 25

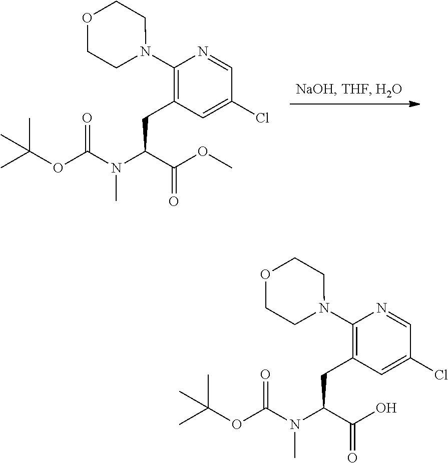

A solution of methyl (2S)-2-[(tert-butoxycarbonyl)(methyl)amino]-3-[5-chloro-2-(morpholin-4-yl)pyridin-3-yl]propanoate (660 mg, 1.60 mmol, 1 eq.) in THF (10 mL) was treated with NaOH (318.89 mg, 7.98 mmol, 5 eq.) in water (10 mL) at 0° C. The resulting mixture was stirred for 2 h at rt. Desired product could be detected by LCMS. The mixture neutralized to pH=2 with diluted HCl (1N). The resulting mixture was extracted with EtOAc (2×50 mL). The combined organic layers were dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (0.1% FA), 10% to 100% gradient in 40 min; detector, UV 200.210 nm. This resulted in Int. 25 (423 mg, 66%) as a white solid. LCMS (ESI+): m/z 400.15. ¹H NMR (400 MHz, DMSO-d₆): δ 1.26 (s, 9H), 2.61 (d, J=8.7 Hz, 3H), 3.00 (tq, J=13.5, 8.6, 7.2 Hz, 4H), 3.63-3.78 (m, 4H), 5.03 (ddd, J=148.8, 11.4, 4.2 Hz, 1H), 7.67 (dd, J=35.8, 2.6 Hz, 1H), 8.21 (dd, J=14.5, 2.6 Hz, 1H), 12.91 (s, 1H).

### Int. 26: Preparation of (S)-2-((tert-butoxycarbonyl)(methyl)amino)-3-(7-chloro-2,3-dihydrobenzo[b][1,4]dioxin-5-yl)propanoic acid

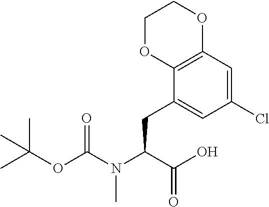 [SMILES: CN(C(=O)OC(C)(C)C)[C@@H](Cc1cc(Cl)cc2c1OCCO2)C(=O)O]

### Step 1: 5-chloro-2-hydroxy-3-nitrophenyl acetate

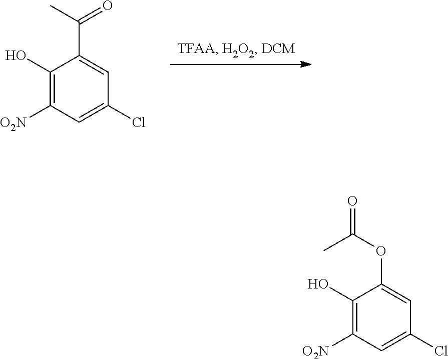

Add TFAA (409.17 g, 1.95 mol, 270.97 mL, 10 eq.) to a three-necked flask, add H₂O₂ (110.44 g, 974.07 mmol, 93.59 mL, 30% purity, 5 eq.) dropwise at −10° C. After 10 min, add a solution of 1-(5-chloro-2-hydroxy-3-nitrophenyl)ethan-1-one (42 g, 194.81 mmol, 1 eq) in DCM (250 mL) dropwise. The reaction mixture was stirred at 20° C. for 12 h. The reaction was monitored by LCMS, which showed starting material was consumed and one of peak with desired mass was detected. The reaction mixture was diluted with water (100 mL), extracted with DCM (300 mL×3). The combined organics were washed with sodium sulfite aqueous solution (100 mL, 1M) and brine (100 mL), dried over Na₂SO₄, filtered, and concentrated under reduced pressure to give 5-chloro-2-hydroxy-3-nitrophenyl acetate (30 g, 124.36 mmol, 64% yield, 96% purity) as a yellow solid. LCMS (ESI+): m/z 230.0 (M+H⁺).

### Step 2: 5-chloro-3-nitrobenzene-1,2-diol

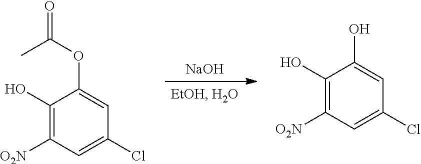

To a solution of 5-chloro-2-hydroxy-3-nitrophenyl acetate (30 g, 129.54 mmol, 1 eq) in EtOH (300 mL) was added NaOH (12.95 g, 129.54 mmol, 40 mL, 40% purity in H₂O, 1 eq), the mixture was stirred at 20° C. for 2 h. The reaction was monitored by LCMS, which showed starting material was consumed and one of peak with desired mass was detected. The reaction mixture was concentrated under reduced pressure to give a residue. The residue was acidified pH=4 by adding hydrochloric acid (1 M, 50 mL) dropwise at 0° C. The mixture was extracted with EtOAc (200 mL×3). The combined organics were washed with brine (50 mL), dried over Na₂SO₄, filtered and concentrated under reduced pressure to give 5-chloro-3-nitrobenzene-1,2-diol (22 g, 113.74 mmol, 87.80% yield, 98% purity) as a yellow solid. LCMS (ESI+): m/z 188.0 (M+H⁺).

### Step 3: 7-chloro-5-nitro-2,3,-dihydrobenzo[b][1,4]dioxine

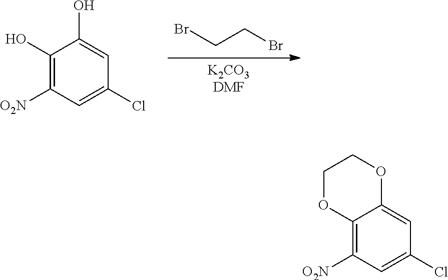

To a solution of 5-chloro-3-nitrobenzene-1,2-diol (10 g, 52.76 mmol, 1 eq) in DMF (100 mL) was added 1,2-dibromoethane (9.91 g, 52.76 mmol, 3.98 mL, 1 eq) and K₂CO₃ (14.58 g, 105.51 mmol, 2 eq) at 20° C. Then the mixture was stirred at 80° C. for 12 h. The reaction was monitored by LCMS, which showed starting material was consumed and one of main peak was detected. The reaction mixture was cooled to rt and diluted by water (100 mL), extracted with EtOAc (60 mL×3). The combined organics were washed with water (100 mL×2). Then the combined organics were washed with brine (200 mL), dried over Na₂SO₄, filtered and the reaction mixture was concentrated under reduced pressure to give 7-chloro-5-nitro-2,3,-dihydrobenzo[b][1,4]dioxine (11 g, 51.0 mmol, 97% yield) as a black, brown oil.

### Step 4: 7-chloro-2,3-dihydrobenzo[b][1,4]dioxin-5-amine

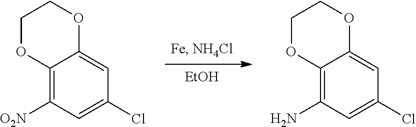

To a solution of 7-chloro-5-nitro-2,3,-dihydrobenzo[b][1,4]dioxine (16 g, 74.21 mmol, 1 eq) in EtOH (80 mL) and H₂O (80 mL) were added Fe (20.72 g, 371.07 mmol, 5 eq) and NH₄Cl (19.85 g, 371.07 mmol, 5 eq) at 20° C. The reaction was stirred at 80° C. for 2 h. The reaction was monitored by LCMS, which showed starting material was consumed and one of main peak with desired mass was detected. The reaction mixture was filtered through a pad of celite and the celite was rinsed with EtOAc (100 mL). The filtrate was diluted with water (100 mL), extracted with EtOAc (120 mL×3). The combined organics were washed with brine (200 mL), dried over Na₂SO₄, filtered, and concentrated under reduced pressure to give a residue. The residue was dissolved in DCM (200 mL), and 50 g of silica gel was added. The resulting mixture was evaporated under reduced pressure to give a dry flowing solid, and then it was loaded to Biotage® using an 80 g Agela flash silica gel column, eluted with 30% to 40% EtOAc in PE with the flower rate of 75 mL/min. The product fraction was combined and evaporated to give 7-chloro-2,3-dihydrobenzo[b][1,4]dioxin-5-amine (8.6 g, 43.84 mmol, 30% yield, 94% purity) as a brown oil. LCMS (ESI+): m/z 186.3 (M+H⁺).

### Step 5: 7-chloro-5-iodo-2,3-dihydrobenzo[b][1,4]dioxine

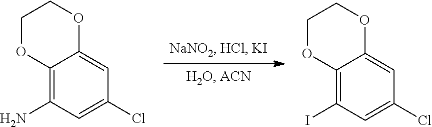

To a solution of 7-chloro-2,3-dihydrobenzo[b][1,4]dioxin-5-amine (4.6 g, 24.78 mmol, 1 eq) in MeCN (24 mL) was added hydrogen chloride (2.5 M, 49.57 mL, 5 eq) at 0° C. The mixture was stirred at 0° C. for 0.5 h. Then a solution of NaNO₂ (1.88 g, 27.26 mmol, 1.1 eq) in H₂O (20 mL) was added to the reaction mixture, the mixture was stirred at 0° C. for 0.5 h. Then a solution of KI (12.34 g, 74.35 mmol, 3 eq) in H₂O (100 mL) was added to the reaction mixture, the reaction mixture was stirred at 0° C. for 2 h. The reaction was monitored by LCMS, which showed starting material was consumed and one of main peak with desired mass was detected. The mixture was adjusted pH=7-8 by adding saturated NaHCO₃ aqueous solution slowly at 0° C. Then the mixture was extracted with EtOAc (50 mL×3). The combined organics were washed with brine (100 mL), dried over Na₂SO₄, filtered, and concentrated under reduced pressure to give a residue. The residue was dissolved in DCM (50 mL), and 20 g of silica gel was added. The resulting mixture was evaporated under reduced pressure to give a dry flowing solid, and then it was loaded to Biotage® using a 40 g Agela flash silica gel column, eluted with 15% to 20% EtOAc in PE with the flower rate of 75 mL/min. The product fraction was combined and evaporated to give 7-chloro-5-iodo-2,3-dihydrobenzo[b][1,4]dioxine (6.5 g, 21.71 mmol, 88% yield, 99% purity) as a black, purple solid. LCMS (ESI+): m/z 298.1 (M+H⁺).

### Step 6: methyl(S)-2-((tertbutoxycarbonyl)amino)-3-(7-chloro-2,3-dihydrobenzo[b][1,4]dioxin-5-yl)propanoate

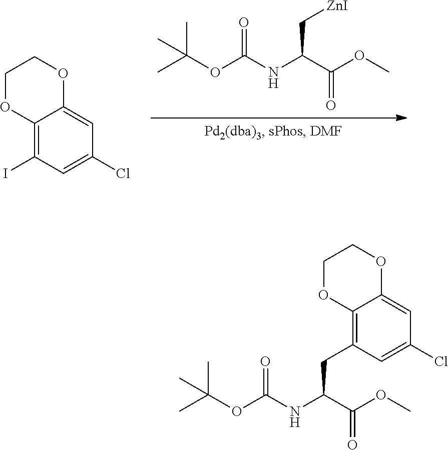

A mixture of Zn (13.91 g, 212.68 mmol, 3.5 eq) in DMF (100 mL) was degassed and purged with N₂ for 3 times, and the mixture was stirred at 120° C. for 10 min under N₂ atmosphere. Then I₂ (4.63 g, 18.23 mmol, 3.7 mL, 0.3 eq) in DMF (40 mL) was added dropwise at 20° C., methyl (2R)-2-(tert-butoxycarbonylamino)-3-iodo-propanoate (20 g, 60.77 mmol, 1 eq) in DMF (30 mL) was added dropwise, I₂ (4.63 g, 18.23 mmol, 3.67 mL, 0.3 eq) in DMF (30 mL) was added dropwise, the reaction mixture was stirred at 20° C. for 1 hr. The zincate was used into the next step without further purification. [(2R)-2-(tert-butoxycarbonylamino)-3-methoxy-3-oxo-propyl]-iodo-zinc (23.95 g, 60.71 mmol, 99.9% yield) was obtained as a gray liquid. To a solution of 7-chloro-5-iodo-2,3-dihydrobenzo[b][1,4]dioxine (9 g, 30.36 mmol, 1 eq) in DMF (90 mL) was added [(2R)-2-(tert-butoxycarbonylamino)-3-methoxy-3-oxo-propyl]-iodo-zinc (23.95 g, 60.71 mmol, 2 eq), Pd₂(dba)₃ (1.39 g, 1.52 mmol, 0.05 eq) and SPhos (1.25 g, 3.04 mmol, 0.1 eq) at 20° C. Then the mixture was stirred at 20° C. for 12 h. The reaction was monitored by LCMS, which showed starting material was consumed and one of main peak with desired mass was detected. The reaction mixture was diluted by water (90 mL), extracted with EtOAc (100 mL×3). The combined organics were washed with water (150 mL×2). Then the combined organics were washed with brine (200 mL), dried over Na₂SO₄, filtered and the reaction mixture was concentrated under reduced pressure to give a residue. The residue was dissolved in DCM (30 mL), and 18 g of silica gel was added. The resulting mixture was evaporated under reduced pressure to give a dry flowing solid, and then it was loaded to Biotage® using a 40 g Agela flash silica gel column, eluted with 0% to 20% EtOAc in PE with the flower rate of 75 mL/min. The product fraction was combined and evaporated to give methyl(S)-2-((tertbutoxycarbonyl)amino)-3-(7-chloro-2,3-dihydrobenzo[b][1,4]dioxin-5-yl)propanoate (6 g, 15.55 mmol, 51% yield, 96% purity) a yellow oil.

### Step 7: (S)-2-((tert-butoxycarbonyl)amino)-3-(7-chloro-2,3-dihydrobenzo[b][1,4]dioxin-5-yl)propanoic acid

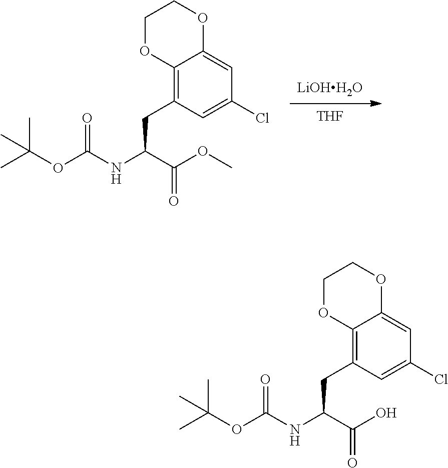 [SMILES: COC(=O)[C@H](Cc1cc(Cl)cc2c1OCCO2)NC(=O)OC(C)(C)C ; "LiOH·H2O" ; CC(C)(C)OC(=O)N[C@@H](Cc1cc(Cl)cc2c1OCCO2)C(=O)O ; "THF"]

To a solution of methyl(S)-2-((tertbutoxycarbonyl)amino)-3-(7-chloro-2,3-dihydrobenzo[b][1,4]dioxin-5-yl)propanoate (5.5 g, 14.79 mmol, 1 eq) in THF (36 mL) was added a solution of LiOH·H₂O (744.89 mg, 17.75 mmol, 1.2 eq) in H₂O (18 mL) at 0° C. The mixture was stirred at 20° C. for 2 h. The reaction was monitored by LCMS, which showed starting material was consumed and one of main peak with desired mass was detected. The mixture was acidified by adding hydrochloric acid (1 M) dropwise at 0° C. to pH=3. The mixture was extracted with EtOAc (50 mL×3). The combined organics were washed with brine (80 mL), dried over Na₂SO₄, filtered, and concentrated under reduced pressure to give a residue. The residue was dissolved in DCM (30 mL), and 10 g of silica gel was added. The resulting mixture was evaporated under reduced pressure to give a dry flowing solid, and then it was loaded to Biotage® using a 40 g Agela flash silica gel column, eluted with 0% to 35% EtOAc in PE with the flower rate of 75 mL/min. The product fraction was combined and evaporated to give (S)-2-((tert-butoxycarbonyl)amino)-3-(7-chloro-2,3-dihydrobenzo[b][1,4]dioxin-5-yl)propanoic acid (5.2 g, 14.53 mmol, 98% yield, 100% purity) as a yellow oil. LCMS (ESI+): m/z 380.2 (M+Na⁺).

### Step 8: Int. 26

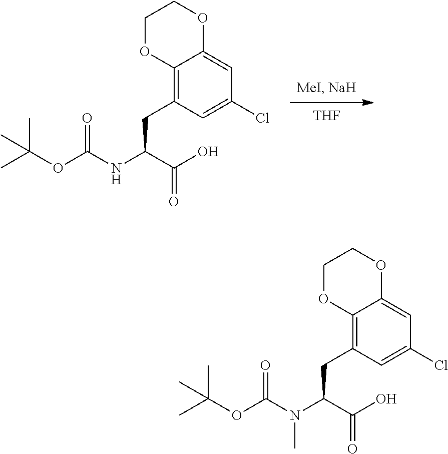

To a solution of (S)-2-((tert-butoxycarbonyl)amino)-3-(7-chloro-2,3-dihydrobenzo[b][1,4]dioxin-5-yl)propanoic acid (2.6 g, 7.27 mmol, 1 eq.) in THF (60 mL) was added NaH (1.45 g, 36.33 mmol, 60% purity, 5 eq) at 0° C., the mixture was stirred at 0° C. for 1 h under N₂. MeI (10.31 g, 72.67 mmol, 4.52 mL, 10 eq.) was added to the reaction mixture, the mixture was stirred at 20° C. for 12 h under N₂. (Batch 2) The reaction was monitored by LCMS, which showed starting material was consumed and one of main peaks with desired mass was detected. The reaction was quenched by saturated ammonium chloride (30 mL) at 0° C., extracted with EtOAc (60 mL×2). The combined organics were washed with brine (50 mL), dried over Na₂SO₄, filtered and the reaction mixture was concentrated under reduced pressure to give Int. 26 (3.9 g, 10.0 mmol, 69% yield, 96% purity) as a pale-yellow solid.). LCMS (ESI+): m/z 394.1 (M+Na⁺). ¹H NMR (400 MHz, CHLOROFORM-d). δ ppm 1.40 (d, J=10.8 Hz, 9H) 2.67-2.82 (m, 3H) 3.00-3.13 (m, 1H) 3.15-3.35 (m, 1H) 4.18-4.34 (m, 4H) 4.61-4.91 (m, 1H) 6.68 (d, J=11.6 Hz, 1H) 6.79 (d, J=2.0 Hz, 1H).

### Int. 27: Preparation of (S)-2-((tert-butoxycarbonyl)(methyl)amino)-3-(5-chloro-2-methylpyridin-3-yl)propanoic acid

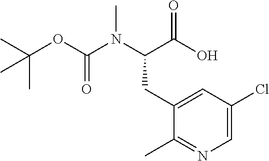 [SMILES: Cc1ncc(Cl)cc1C[C@@H](C(=O)O)N(C)C(=O)OC(C)(C)C]

### Step 1: (5-chloro-2-methylpyridin-3-yl)methanol

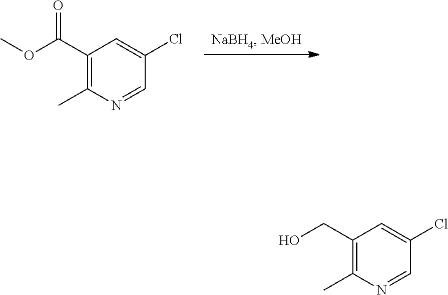

NaBH₄ (6.11 g, 161.63 mmol, 3 eq.) was added into a mixture of methyl 5-chloro-2-methylpyridine-3-carboxylate (10 g, 53.88 mmol, 1 eq.) in MeOH (200 mL) at 0° C. under air atmosphere. The mixture was stirred overnight at rt. Then the reaction was quenched with water (100 mL) and concentrated in vacuo. The residual water phase was extracted with EtOAc (2×200 mL). The combined organic layers were dried over anhydrous Na₂SO₄ and filtered. The filtrate was concentrated under vacuum. The residue was purified by silica gel column chromatography, eluted with PE/EA (10:1) to afford (5-chloro-2-methylpyridin-3-yl)methanol (6.1 g, 72%) as a yellow oil. LCMS (ESI+): m/z 158.0.

### Step 2: 3-(bromomethyl)-5-chloro-2-methylpyridine

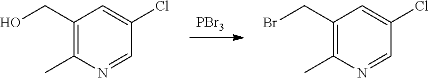

PBr₃ (31431.18 mg, 116.12 mmol, 3 eq.) was added into a mixture of (5-chloro-2-methylpyridin-3-yl)methanol (6.1 g, 38.71 mmol, 1 eq.) in DCM (50 mL) dropwise at 0° C. under nitrogen atmosphere. The mixture was stirred for 3 h at 0° C. The resulting mixture was quenched with water (50 mL) at 0° C. and extracted with EtOAc (2×100 mL). The combined organic layers were dried over anhydrous Na₂SO₄ and filtered. The filtrate was concentrated under vacuum. This resulted in 3-(bromomethyl)-5-chloro-2-methylpyridine (7 g, 82%) as a yellow oil. The crude product was used in the next step directly without further purification. LCMS (ESI+): m/z 222.1.

### Step 3: Tert-butyl (2S)-3-(5-chloro-2-methylpyridin-3-yl)-2-[(diphenylmethylidene)amino]propanoate

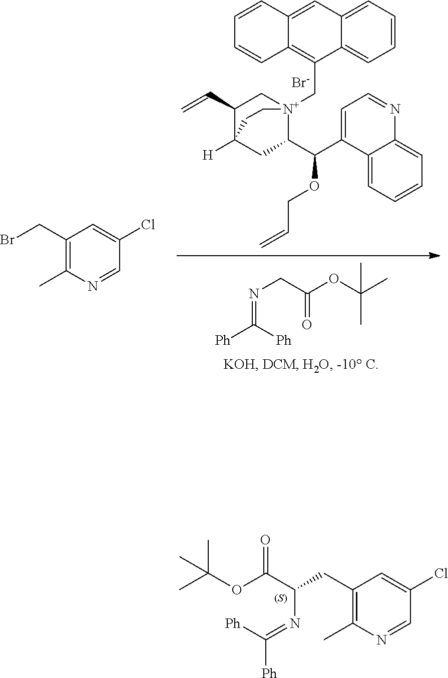

To a stirred mixture of 3-(bromomethyl)-5-chloro-2-methylpyridine (6.1 g, 27.67 mmol, 1 eq.) and tert-butyl 2-[(diphenylmethylidene)amino]acetate (12257.90 mg, 41.50 mmol, 1.5 eq.) in DCM (60 mL) was added KOH (15521.82 mg, 276.66 mmol, 10 eq.) in H₂O (15 mL) in portions at −10° C. under air atmosphere. The resulting mixture was stirred overnight at −10° C. The resulting mixture was extracted with DCM (2×100 mL) and the combined organic layers were dried over anhydrous Na₂SO₄ and filtered. The filtrate was concentrated under vacuum. The residue was purified by silica gel column chromatography, eluted with PE/EA (10:1) to afford tert-butyl (2S)-3-(5-chloro-2-methylpyridin-3-yl)-2-[(diphenylmethylidene)amino]propanoate (6.4 g, 53%) as a yellow oil. LCMS (ESI+): m/z 435.0.

### Step 4: (S)-2-amino-3-(5-chloro-2-methylpyridin-3-yl)propanoic acid

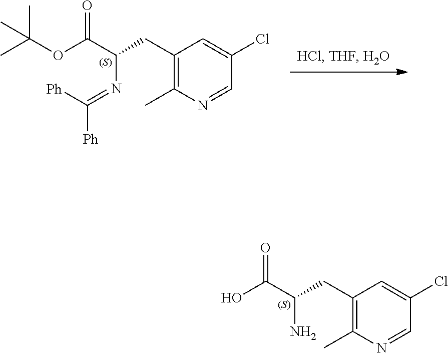

A mixture of tert-butyl (2S)-3-(5-chloro-2-methylpyridin-3-yl)-2-[(diphenylmethylidene)amino]propanoate (300 mg, 0.69 mmol, 1 eq.) and HCl (6M) (100.00 mL, 3291.28 mmol, 119.30 eq.) in THF (10 mL) was stirred overnight at 55° C. under air atmosphere. The resulting mixture was concentrated under vacuum. This resulted in (2S)-2-amino-3-(5-chloro-2-methylpyridin-3-yl) propanoic acid (5 g, 84.43%) as a yellow oil. The crude product was used in the next step directly without further purification. LCMS (ESI+): m/z 215.0.

### Step 5: (2S)-2-[(tert-butoxycarbonyl)amino]-3-(5-chloro-2-methylpyridin-3-yl)propanoic acid

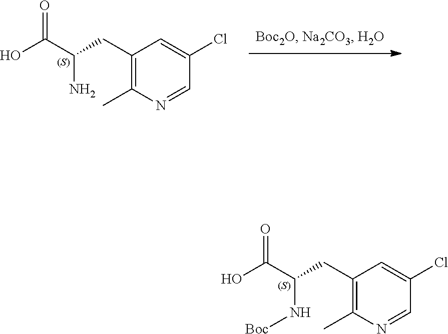

To a stirred mixture of (2S)-2-amino-3-(5-chloro-2-methylpyridin-3-yl)propanoic acid (4 g, 18.64 mmol, 1 eq.) and Boc₂O (12.20 g, 55.91 mmol, 3.00 eq.) in H₂O (100 mL) was added Na₂CO₃ (5.93 g, 55.91 mmol, 3 eq.) in portions at rt under air atmosphere. The resulting mixture was stirred overnight at rt. The mixture was acidified to pH=6 with diluted HCl (aq. 1N). The resulting mixture was stirred overnight at 0° C. The resulting mixture was extracted with EtOAc (2×100 mL). The combined organic layers were dried over anhydrous Na₂SO₄, filtered and concentrated in vacuo. The residue was purified by reversed-phase flash chromatography with the following conditions: column, C18 silica gel; mobile phase, MeCN in Water (0.1% TFA), 10% to 100% gradient in 35 min; detector, UV 220 nm. This resulted in (2S)-2-[(tert-butoxycarbonyl)amino]-3-(5-chloro-2-methylpyridin-3-yl)propanoic acid (5.6 g, 95%) as a white solid. LCMS (ESI+): m/z 315.10. ¹H NMR (400 MHz, DMSO-d₆): δ12.82 (s, 1H), 8.33 (d, J=2.4 Hz, 1H), 7.65 (d, J=2.5 Hz, 1H), 7.21 (d, J=8.8 Hz, 1H), 4.28-4.10 (m, 1H), 3.11 (dd, J=14.3, 4.4 Hz, 1H), 2.81 (dd, J=14.4, 11.0 Hz, 1H), 2.47 (s, 3H), 1.30 (s, 9H).

### Step 6: Int. 27

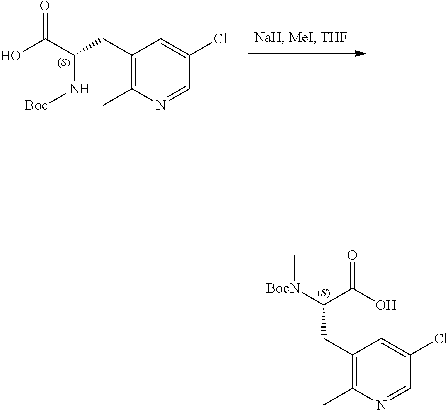

NaH (1.19 g, 49.56 mmol, 6 eq.) was added into a stirred mixture of (2S)-2-[(tert-butoxycarbonyl)amino]-3-(5-chloro-2-methylpyridin-3-yl)propanoic acid (2.6 g, 8.26 mmol, 1 eq.) in THF (25 mL) portion wise at 0° C. under nitrogen. Then the mixture was stirred for 1 h at 0° C. MeI (11.72 g, 82.60 mmol, 10 eq.) was added into the above solution at 0° C. under nitrogen atmosphere. The resulting mixture was stirred overnight at rt. The reaction was quenched by the addition of sat. NH₄Cl (aq.) (20 mL) at 0° C. The mixture was acidified to pH=6 with diluted HCl (aq. 1N). The resulting mixture was extracted with EtOAc (2×50 mL), and the combined organic layers were dried over anhydrous Na₂SO₄ and filtered. The residue was purified by reversed-phase flash chromatography with the following conditions: column, C18 silica gel; mobile phase, MeCN in Water (0.1% FA), 10% to 100% gradient in 35 min; detector, UV 210 nm. This resulted in Int. 27 (3.71 g, 133% mass recovery) as a yellow solid. LCMS (ESI+): m/z 329.10. ¹H NMR (400 MHz, DMSO-d₆): δ 12.98 (s, 1H), 8.34 (dd, J=16.6, 2.4 Hz, 1H), 7.59 (dd, J=21.9, 2.4 Hz, 1H), 4.72 (ddd, J=53.9, 11.2, 4.4 Hz, 1H), 3.27-2.92 (m, 2H), 2.65 (d, J=2.0 Hz, 3H), 2.50 (s, 3H), 1.25 (d, J=34.2 Hz, 9H).

### Int. 28: Preparation of (S)-2-((tert-butoxycarbonyl)(methyl)amino)-3-(5-chloro-2-((1-methyl-1H-pyrazol-4-yl)oxy)pyridine-3-yl)propanoic acid

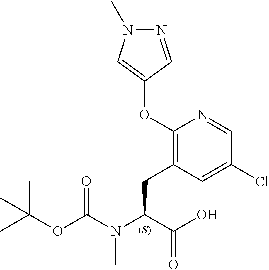

### Step 1: 5-chloro-3-iodo-2-((1-methyl-1H-pyrazol-4-yl)oxy)pyridine

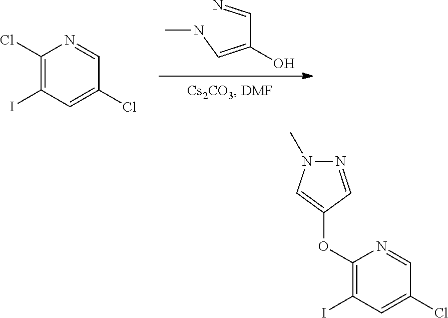

To a solution of 2,5-dichloro-3-iodopyridine (10 g, 36.51 mmol, 1 eq) in DMF (140 mL) was added Cs₂CO₃ (23.79 g, 73.02 mmol, 2 eq) and 1-methylpyrazol-4-ol (4.30 g, 43.81 mmol, 1.2 eq). The mixture was stirred at 80° C. for 1 hr. LCMS showed 2,5-dichloro-3-iodopyridine was consumed completely and desired mass was detected. After cooling to rt, the reaction mixture was diluted with water (200 mL), extracted with EtOAc (100 mL×3). The combined organic layers were washed with brine (100 mL), dried over Na₂SO₄, filtered, and concentrated under reduced pressure to give a residue. The resulting crude product was purified by flash chromatography on silica gel (0-22% EtOAc in PE). 5-chloro-3-iodo-2-((1-methyl-1H-pyrazol-4-yl)oxy)pyridine (10.9 g, 29.59 mmol, 81% yield, 91% purity) was obtained as a yellow solid.

### Step 2: methyl(S)-2-((tert-butoxycarbonyl)amino)-3-(5-chloro-2-((1-methyl-1H-pyrazol-4-yl)oxy)pyridine-3-yl)propanoate

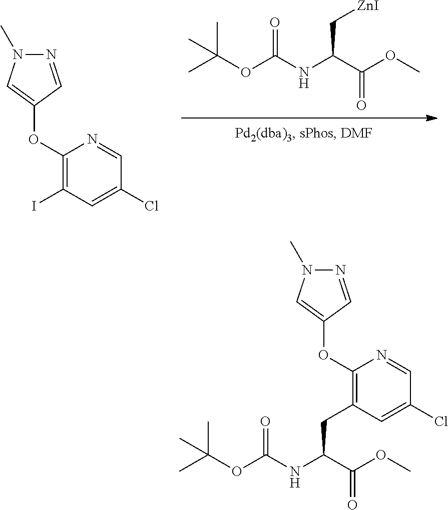

A mixture of Zn (18.08 g, 276.49 mmol, 3.5 eq) in DMF (150 mL) was degassed and purged with N₂ for 3 times, and the mixture was stirred at 120° C. for 10 min under N₂ atmosphere. Then I₂ (6.01 g, 23.70 mmol, 4.77 mL, 0.3 eq) in DMF (20 mL) was added dropwise at 20° C., methyl (R)-2-((tert-butoxycarbonyl)amino)-3-iodopropanoate (26 g, 79.00 mmol, 1 eq) in DMF (70 mL) was added dropwise, I₂ (6.01 g, 23.70 mmol, 4.77 mL, 0.3 eq) in DMF (20 mL) was added dropwise, the reaction mixture was stirred at 20° C. for 1 hr. The zincate was used directly.

To a solution of 5-chloro-3-iodo-2-((1-methyl-1H-pyrazol-4-yl)oxy)pyridine (9.9 g, 29.51 mmol, 1 eq) in DMF (260 mL) was added Pd₂(dba)₃ (1.35 g, 1.48 mmol, 0.05 eq) and sPhos (1.21 g, 2.95 mmol, 0.1 eq) under N₂ atmosphere, and then was added dropwise, (R)-(2-((tert-butoxycarbonyl)amino)-3-methoxy-3-oxopropyl)zinc(II) iodide (24.33 g in DMF, 61.67 mmol, 2.09 eq), under N₂ atmosphere and the reaction was stirred at 65° C. for 12 hr under N₂. LCMS showed 5-chloro-3-iodo-2-((1-methyl-1H-pyrazol-4-yl)oxy)pyridine was consumed completely and desired mass was detected. The mixture was quenched by NH₄Cl (200 mL) and extracted with EtOAc (200 mL×3). The combined organic layers were washed with brine (100 mL), dried over Na₂SO₄, filtered, and concentrated under reduced pressure to give a residue. The resulting crude product was purified by flash chromatography on silica gel (0-21% EtOAc in PE) to give methyl(S)-2-((tert-butoxycarbonyl)amino)-3-(5-chloro-2-((1-methyl-1H-pyrazol-4-yl)oxy)pyridine-3-yl)propanoate (5.6 g, 11.97 mmol, 41% yield, 87.8% purity) as a pale-yellow gum.

### Step 3: (S)-2-((tert-butoxycarbonyl)amino)-3-(5-chloro-2-((1-methyl-1H-pyrazol-4-yl)oxy)pyridine-3-yl)propanoic acid

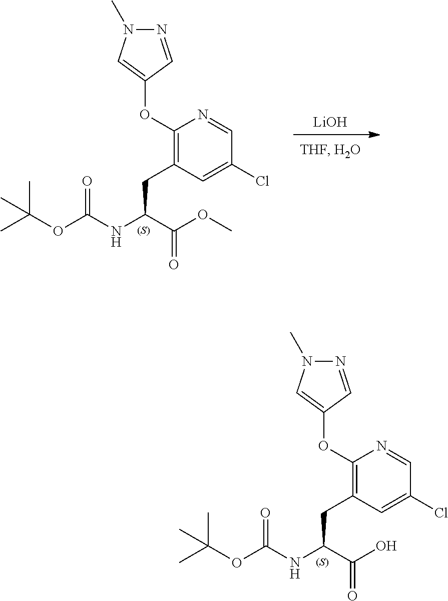

To a solution of methyl(S)-2-((tert-butoxycarbonyl)amino)-3-(5-chloro-2-((1-methyl-1H-pyrazol-4-yl)oxy)pyridine-3-yl)propanoate (4.6 g, 11.20 mmol, 1 eq) in THF (40 mL) was added LiOH·H₂O (704.75, 16.79 mol, 1499.99 eq) in H₂O (20 mL) at 0° C. and the reaction was stirred at 25° C. for 0.5 hr. LCMS showed methyl(S)-2-((tert-butoxycarbonyl)amino)-3-(5-chloro-2-((1-methyl-1H-pyrazol-4-yl)oxy)pyridine-3-yl)propanoate was consumed completely and desired mass was detected. The reaction mixture was washed with MTBE (10 mL×2), then the aqueous phase was adjusted pH to 3-4 with 1M HCl, extracted with EtOAc (30 mL×3). The combined organic layers were washed with brine (20 mL), dried over Na₂SO₄, filtered and concentrated under reduced pressure to give a residue, (S)-2-((tert-butoxycarbonyl)amino)-3-(5-chloro-2-((1-methyl-1H-pyrazol-4-yl)oxy)pyridine-3-yl)propanoic acid (4 g, 9.37 mmol, 84% yield, 93% purity) was obtained as a pale yellow gum without purification.

### Step 4: Int. 28

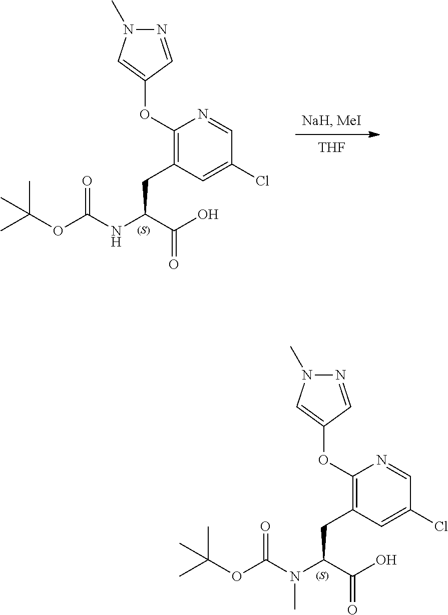

To a solution of (S)-2-((tert-butoxycarbonyl)amino)-3-(5-chloro-2-((1-methyl-1H-pyrazol-4-yl)oxy)pyridine-3-yl)propanoic acid (3 g, 7.56 mmol, 1 eq) in THF (90 mL) was added NaH (1.51 g, 37.80 mmol, 60% purity, 5 eq) at 0° C. under N₂ atmosphere, the mixture was stirred at 0° C. for 30 min then was added MeI (10.73 g, 75.60 mmol, 4.71 mL, 10 eq). The mixture was warmed to 25° C. and stirred at 25° C. for 12 hr. LCMS showed (S)-2-((tert-butoxycarbonyl)amino)-3-(5-chloro-2-((1-methyl-1H-pyrazol-4-yl)oxy)pyridine-3-yl)propanoic acid was consumed completely and desired mass was detected. The reaction mixture was quenched with NH₄Cl (100 mL), adjusted pH to 3-4 with 1M HCl, extracted with EtOAc (30 mL×4). The combined organics were washed with brine (50 mL), dried over Na₂SO₄, filtered, and concentrated under reduced pressure to give a residue. The residue was diluted with MeOH (50 mL) and washed with PE (20 mL×4). The residue was concentrated under reduced pressure to give pale yellow solid. Int. 28 (2.6 g, 6.10 mmol, 81% yield, 92.8% purity) was obtained as a pale-yellow solid without purification.

### Int. 29: (S)-3-(2-((1-acetylazetidin-3-yl)oxy)-5-chloropyridin-3-yl)-2-((tert-butoxycarbonyl)(methyl)amino)propanoic acid

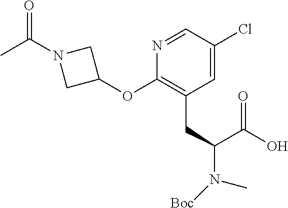 [SMILES: CC(=O)N1CC(Oc2ncc(Cl)cc2C[C@@H](C(=O)O)N(C)C(=O)OC(C)(C)C)C1]

This material was synthesized by following the procedure for Int. 13 with 1-(3-hydroxyazetidin-1-yl)ethan-1-one as the starting alcohol. LCMS (ESI+): m/z 428.1 (M+H⁺)

### Int. 30: Preparation of (S)-3-(2-bromo-5-chlorophenyl)-2-((tert-butoxycarbonyl)(methyl)amino)propanoic acid

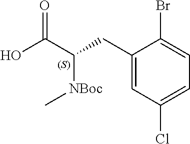

### Step 1: (2S,5R)-2-(2-bromo-5-chlorobenzyl)-3,6-diethoxy-5-isopropyl-2,5-dihydropyrazine

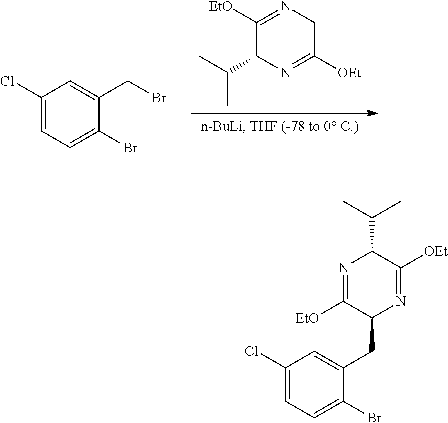 [SMILES: Clc1ccc(Br)c(CBr)c1 ; CCOC1=N[C@H](C(C)C)C(OCC)=NC1 ; "n-BuLi, THF (-78 to 0° C.)" ; CCOC1=N[C@H](C(C)C)C(OCC)=N[C@H]1Cc1cc(Cl)ccc1Br]

To a solution of (R)-3,6-diethoxy-2-isopropyl-2,5-dihydropyrazine (151 g, 711 mmol, 1.00 eq) in THF (1500 mL) was added n-BuLi (2.5 M, 285 mL, 1.00 eq) at −70° C. under N₂, then to the above solution was added 1-bromo-2-(bromomethyl)-4-chlorobenzene (222.5 g, 782 mmol, 1.10 eq) in THF (220 mL) at −70° C. The mixture was stirred at 25° C. for 12 hours. TLC indicated 1-bromo-2-(bromomethyl)-4-chlorobenzene was remained, and some new spots was detected. The reaction was poured into sat. NH₄Cl (aq.) 6000 mL, the reaction mixture was partitioned between water phase 6000 mL and THF 6000 mL. The organic phase was separated and dried over Na₂SO₄, filtered, and concentrated under reduced pressure to give a residue. The residue was purified by column chromatography (SiO2, PE/EtOAc=100/0 to 1/1) to give desired (2S,5R)-2-(2-bromo-5-chlorobenzyl)-3,6-diethoxy-5-isopropyl-2,5-dihydropyrazine (620 g, 1.48 mol, 104% yield, 99.3% purity) as a yellow gum. LCMS (ESI+): m/z 415/417 (M+H⁺)

### Step 2: Ethyl(S)-2-amino-3-(2-bromo-5-chlorophenyl)propanoate

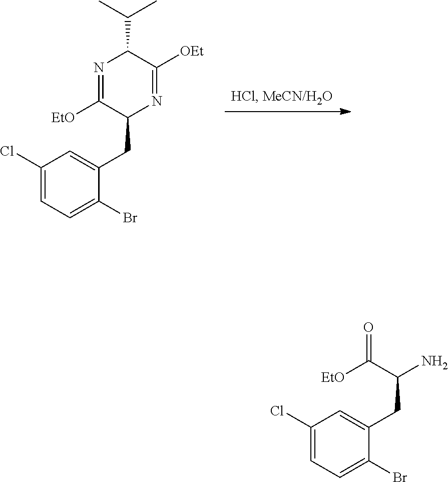 [SMILES: CCOC1=N[C@H](C(C)C)C(OCC)=N[C@H]1Cc1cc(Cl)ccc1Br ; "HCl, MeCN/H2O" ; CCOC(=O)[C@@H](N)Cc1cc(Cl)ccc1Br]

To a solution of (2S,5R)-2-(2-bromo-5-chlorobenzyl)-3,6-diethoxy-5-isopropyl-2,5-dihydropyrazine (100 g, 240 mmol, 1.00 eq) in ACN (1500 mL) was added HCl (0.20 M, 1.40 L, 1.16 eq) at 0° C., then was stirred at 25° C. for 48 hours. TLC indicated (2S,5R)-2-(2-bromo-5-chlorobenzyl)-3,6-diethoxy-5-isopropyl-2,5-dihydropyrazine was consumed completely. The reaction mixture was concentrated under reduced pressure to remove ACN and extracted with DCM 4000 mL (2000 mL×2), the organic layers was concentrated under reduced pressure to give a residue. Then the water phase was basified with sat. aq. NaHCO₃ to pH=7 and extracted with DCM 4.00 L (2.00 L×2). The combined organic layers were dried over Na₂SO₄, filtered, and concentrated under reduced pressure to give desired ethyl(S)-2-amino-3-(2-bromo-5-chlorophenyl)propanoate (143 g, 466 mmol, 49% yield) as a white solid and to give (2S,5R)-2-(2-bromo-5-chlorobenzyl)-3,6-diethoxy-5-isopropyl-2,5-dihydropyrazine (200 g, crude) was obtained as a yellow gum.

### Step 3: (S)-2-amino-3-(2-bromo-5-chlorophenyl)propanoic acid

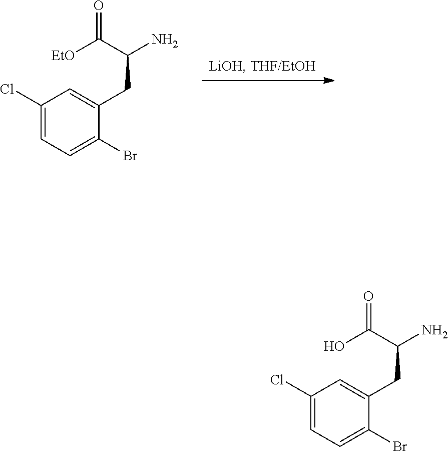 [SMILES: CCOC(=O)[C@@H](N)Cc1cc(Cl)ccc1Br ; "LiOH, THF/EtOH" ; N[C@@H](Cc1cc(Cl)ccc1Br)C(=O)O]

To a solution of ethyl(S)-2-amino-3-(2-bromo-5-chlorophenyl)propanoate (143 g, 466 mmol, 1.00 eq) in THF (715 mL) and EtOH (715 mL) was added LiOH·H2O (58.7 g, 1.40 mol, 3.00 eq) at 0° C., then the reaction was stirred at 25° C. for 12 hours. TLC indicated ethyl(S)-2-amino-3-(2-bromo-5-chlorophenyl)propanoate was consumed completely. The reaction mixture was concentrated under reduced pressure at 30° C. to remove half of solvent. It was added into a saturated solution of citric acid (1.50 L) at 5-10° C., to keep all the progress worked under acid condition. Some solid precipitated, then the suspension was filtered, the filter cake was washed with MTBE (1.00 L). The filter cake was collected and dried in vacuum to give desired (S)-2-amino-3-(2-bromo-5-chlorophenyl)propanoic acid (130 g, crude) as yellow solid which was confirmed by LCMS and SFC, then was used to the next step without further purification.

### Step 4: (S)-3-(2-bromo-5-chlorophenyl)-2-((tert-butoxycarbonyl)amino)propanoic acid

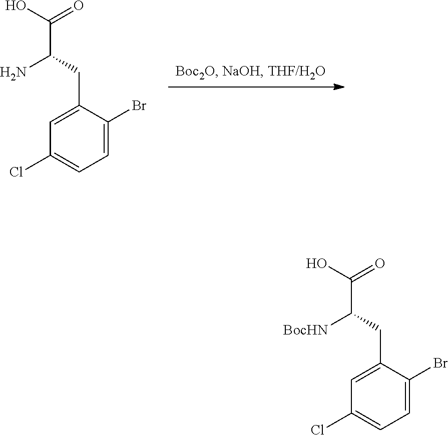 [SMILES: N[C@@H](Cc1cc(Cl)ccc1Br)C(=O)O ; "Boc2O, NaOH, THF/H2O" ; CC(C)(C)OC(=O)N[C@@H](Cc1cc(Cl)ccc1Br)C(=O)O]

To a mixture of (2S)-2-amino-3-(2-bromo-5-chloro-phenyl)propanoic acid (70.00 g, 251.32 mmol, 1 eq) in THF (750 mL) and NaOH (1 N, 750 mL) was added Boc₂O (109.70 g, 502.64 mmol, 115.47 mL, 2 eq) at 0° C., the mixture was stirred at 20° C. for 12 h. The LCMS showed that the reactant 1 was consumed completely, and the desired product was detected. The mixture was poured into sat. Citric acid (4 L) and extracted with EtOAc (1 L×3). The organic layer was washed with water (2 L) and brine (2 L), dried over Na₂SO₄, filtered, and concentrated. The mixture was suspended in PE (1 L) and stirred at 20° C. for 0.5 h. Then the mixture was filtered, and the white solid was collected and dried to dryness to give compound 2 (2S)-3-(2-bromo-5-chloro-phenyl)-2-(tert-butoxycarbonylamino)propanoic acid (124 g, 320.9 mmol, 64% yield, 98% purity) as a white solid. ¹H NMR: ET53616-98-P1Z (400 MHz, MeOD) δ 7.54 (d, J=8.4 Hz, 1H), 7.33 (d, J=2.4 Hz, 1H), 7.17-7.15 (m, 1H), 4.49 (dd, J=4.4, 10.4 Hz, 1H), 3.39 (br dd, J=4.4, 9.6 Hz, 1H), 2.94-2.88 (m, 1H), 1.36-1.28 (m, 9H). LCMS (ESI+): m/z 277.9/279.9 (M+H⁺)

### Step 5: Int. 30

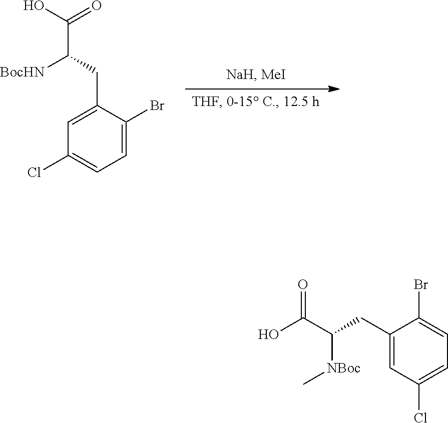

To a mixture of (2S)-3-(2-bromo-5-chloro-phenyl)-2-(tert-butoxycarbonylamino)propanoic acid (3.00 g×10, 7.92 mmol, 1 eq) in THF (90 mL×10) was added NaH (1.58 g×10, 39.61 mmol, 60% purity, 5 eq) at 0° C., the mixture was stirred at 0° C. for 0.5 h. Then MeI (11.25 g×10, 79.23 mmol, 4.93 mL, 10 eq) was added at 0° C., the mixture was stirred at 15° C. for 12 h. The LCMS showed that the (2S)-3-(2-bromo-5-chloro-phenyl)-2-(tert-butoxycarbonylamino)propanoic acid was consumed completely, and the desired product was detected. The mixture was quenched by sat. Citric acid (1.5 L), extracted with EtOAc (1.5 L×2). The organic layer was washed with brine (1.5 L), dried over Na₂SO₄, filtered, and concentrated. The residue was purified by column chromatography (SiO₂, PE/EtOAc/THF=20/1/0 to 6/1/1) to give crude product. The mixture was combined with ET53616-100,104. The residue was dissolved in DCM (1 L) and concentrated under reduced pressure to give the desired product Int. 30 (114.0 g, 285.70 mmol, 98% purity) as a yellow solid. ¹H NMR: ET53616-105-P1Z2 (400 MHz, CHLOROFORM-d) δ 11.44 (brs, 1H), 7.50-7.47 (m, 1H), 7.20-7.19 (m, 1H), 7.13-7.11 (m, 1H), 4.83-4.71 (m, 1H), 3.48-3.43 (m, 1H), 3.31-3.06 (m, 1H), 2.81-2.70 (m, 3H), 1.44-1.37 (m, 9H). LCMS (ESI+): m/z 291.9/293.9 (M+H⁺).

### Int. 31: (S)-3-(2-bromo-5-fluorophenyl)-2-((tert-butoxycarbonyl)(methyl)amino)propanoic acid

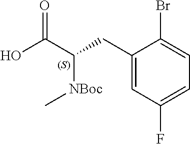

This material was synthesized by following the procedure for Int. 30 acid using 1-bromo-2-(bromomethyl)-4-fluorobenzene as the starting material.

### Int. 33: ((2S,4R)-4-fluoro-1-(1-(trifluoromethyl)cyclopropane-1-carbonyl)pyrrolidine-2-carbonyl)glycine

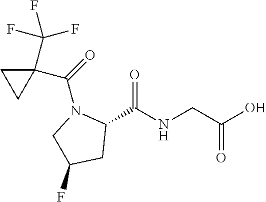 [SMILES: O=C(O)CNC(=O)[C@@H]1C[C@@H](F)CN1C(=O)C1(C(F)(F)F)CC1]

This material was synthesized in an identical way to Int. 34 using commercially available FMOC protected amino acids. LCMS (ESI+): m/z 327.1 (M+H⁺).

### Int. 34: Preparation of ((S)-2-(1-(trifluoromethyl)cyclopropane-1-carboxamido)butanoyl)-L-alanine

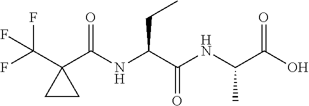 [SMILES: CC[C@H](NC(=O)C1(C(F)(F)F)CC1)C(=O)N[C@@H](C)C(=O)O]

### Steps 1-4: Int. 34

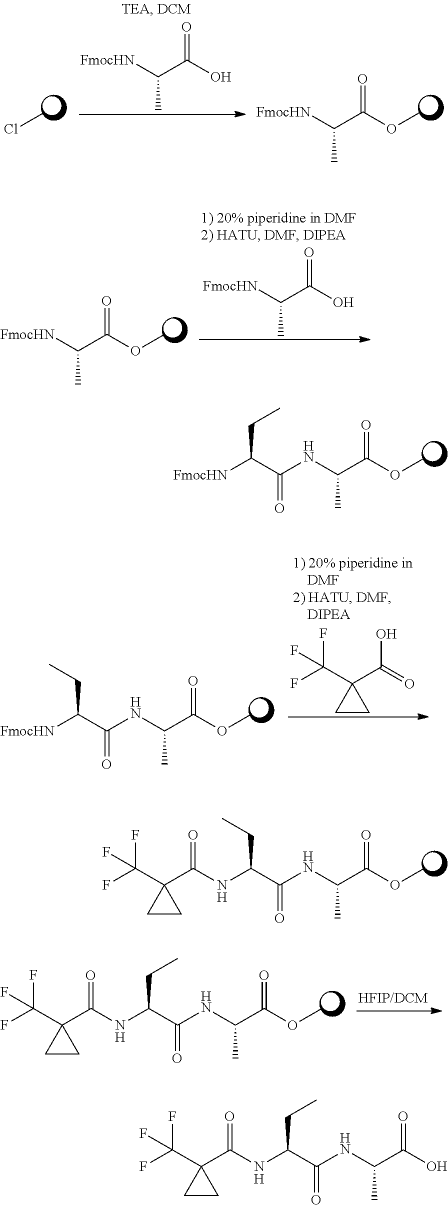

A mix of TEA (7.61 g, 10.5 mL, 6 Eq, 75.2 mmol) and (((9H-fluoren-9-yl)methoxy)carbonyl)-L-alanine (3.90 g, 1 Eq, 12.5 mmol) in DCM (50 mL) was added to a pre-swelled 2-Chlorotrityl chloride resin (5 g). The reaction was agitated for 1 h, then filtered. The loaded resin was washed with DMF (3×50 mL). Next, piperidine 20% in DMF (50 mL) was added to the resin and stirred for 1 h, then washed with DMF (3×50 mL). The freshly deprotected resin was next subjected to a pre-activated solution of Fmoc-protected amino acid consisting of (S)-2-((((9H-fluoren-9-yl)methoxy)carbonyl)amino)butanoic acid (4.08 g, 1 eq, 12.5 mmol), HATU (5.24 g, 1.1 Eq, 13.8 mmol) DIPEA (13.0 g, 17.5 mL, 8 Eq, 100 mmol) in DMF (50 mL). The Resin was agitated for 1 h, then washed with DMF (3×50 mL). The deprotection was repeated with the addition of piperidine 20% in DMF (50 mL). The resin was agitated for 1 h, then washed with DMF (3×50 mL). The final coupling was performed by adding another preactivated carboxylic acid solution, consisting of 1-(trifluoromethyl)cyclopropane-1-carboxylic acid (1.93 g, 1 Eq, 12.5 mmol), HATU (5.24 g, 1.1 Eq, 13.8 mmol), and DIPEA (13.0 g, 17.5 mL, 8 Eq, 100 mmol) in DMF (50 mL), to the resin. This was agitated for 1 h, then washed with DMF (3×50 mL), Methanol (3×50 mL), then DCM (3×50 mL). The tripeptide was then cleaved by agitating the resin with a HFIP/DCM mixture (1:1, 2×50 mL). The combined cleavage filtrate was concentrated and the crude residue was purified by FCC (SiO2, MeOH in DCM 0 to 20% over 10 min.). Int. 34 (500 mg, 80% based on 0.5 mmol/resin loading) was isolated as a white solid. LCMS (ESI+): m/z 311.1 (M+H⁺).

### Int. 35: (S)-2-((2S,4R)-4-fluoro-1-(1-trifluoromethyl)cyclopropane-1-carbonyl)pyrrolidine-2-carboxamido)butanoic acid

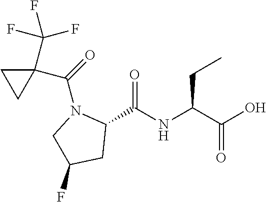 [SMILES: CC[C@H](NC(=O)[C@@H]1C[C@@H](F)CN1C(=O)C1(C(F)(F)F)CC1)C(=O)O]

This material was synthesized in an identical way to Int. 34 using commercially available FMOC protected amino acids. LCMS (ESI+): m/z 355.4 (M+H⁺).

### Int. 39: Preparation of (2S,4R)-4-fluoro-1-(2-(trifluoromethyl)tetrahydro-2H-pyran-2-carbonyl)pyrrolidine-2-carboxylic acid

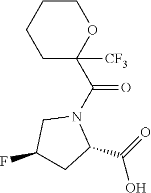 [SMILES: O=C(O)[C@@H]1C[C@@H](F)CN1C(=O)C1(C(F)(F)F)CCCCO1]

Int. 39 was synthesized in accordance with Int. 148 and 149, but without separating the diastereomers.

### Int. 40: Preparation of 1-(4-((4,4,5,5,-tetramethyl-1,3,2-dioxaborolan-2-yl)methylene)piperidin-1-yl)ethan-1-one

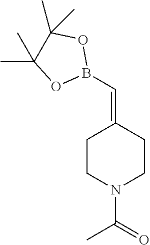 [SMILES: CC(=O)N1CCC(=CB2OC(C)(C)C(C)(C)O2)CC1]

### Step 1: Int. 40

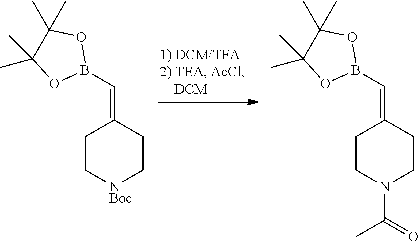 [SMILES: CC(C)(C)OC(=O)N1CCC(=CB2OC(C)(C)C(C)(C)O2)CC1 ; CC(=O)N1CCC(=CB2OC(C)(C)C(C)(C)O2)CC1]

tert-butyl 4-((4,4,5,5-tetramethyl-1,3,2-dioxaborolan-2-yl)methylene)piperidine-1-carboxylate (1.0 g, 1 Eq, 3.1 mmol) was dissolved in 10 mL of DCM/TFA (3:1) and was stirred until the consumption of starting material (monitored by LCMS). The volatile solvent was completely removed and residual TFA was removed via co-evaporation with toluene. The deprotected amine was used directly without further purification. The crude material from above was dissolved in DCM (20 mL) and cooled to 0° C. and TEA (0.94 g, 1.3 mL, 3 eq, 9.3 mmol) and AcCl (0.29 g, 0.26 mL, 1.2 eq, 3.7 mmol) were added subsequently and stirred at 0° C. Upon completion, the volatile solvent was removed, and the resulting solid was placed under high vacuum overnight. The resulting solid Int. 40 (750 mg, 2.83 mmol, 91%) was used in subsequent reactions with no additional purification. LCMS (ESI+): m/z 266.1 (M+H⁺).

### Int. 41: Preparation of (S)-2-((tert-butoxycarbonyl)(ethyl)amino)pent-4-enoic acid

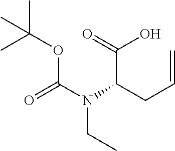 [SMILES: C=CC[C@@H](C(=O)O)N(CC)C(=O)OC(C)(C)C]

### Step 1: (2S)-2-(ethylamino) pent-4-enoic acid

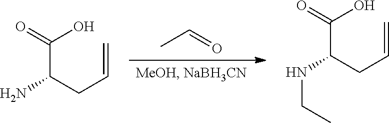

Into a stirred solution of (2S)-2-aminopent-4-enoic acid (5 g, 43.43 mmol, 1 eq) in MeOH (50 mL) was added NaBH₃CN (3.41 g, 54.29 mmol, 1.25 eq) at 0° C. under nitrogen atmosphere. The resulting solution was stirred for 10 min at 0° C. Acetaldehyde (10.97 mL, 195.43 mmol, 1.5 eq) was added and the resulting solution was stirred for 1 h at 0° C. After warming to rt, the mixture was stirred for 16 h at rt. The resulting mixture was quenched with water at 0° C. and extracted with EtOAc (2×10 mL). This resulted in (2S)-2-(ethylamino) pent-4-enoic acid (15 g, 76%) and the crude resulting mixture was used in the next step directly without further purification. LCMS (ESI+): m/z 144.09 (M+H⁺).

### Step 2: Int. 41

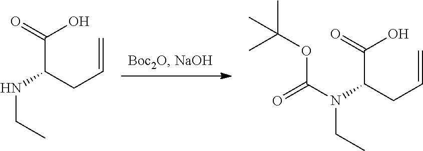

Into a stirred solution of (2S)-2-(ethylamino) pent-4-enoic acid (15 g, 104.76 mmol, 1 eq) and NaOH (29.33 g, 733.31 mmol, 7 eq) in dioxane (150 mL)/H2O (150 mL) was added Boc₂O (45.73 g, 209.52 mmol, 2 eq) at 0° C. under nitrogen atmosphere. The resulting solution was stirred for 16 h at rt. The reaction mixture was concentrated in vacuo to remove dioxane. The aqueous layer was acidified with 1 N HCl to pH=5. The aqueous layer was extracted with EtOAc (3×200 mL). The combined organic layers were washed with brine, dried over Na₂SO₄ and filtered. The filtrate was concentrated in vacuo and the residue was purified by reverse flash chromatography with the following conditions: column, C18 silica gel; mobile phase, MeCN in Water (0.1% FA), 0% to 100% gradient in 40 min; detector, UV 210 nm. This resulted in Int. 41 (11.9 g, 46%) as a white solid. LCMS (ESI+): m/z 244.15 (M+H⁺). ¹H NMR (400 MHz, DMSO-d6): δ 1.05 (dt, J=7.0, 7.0, 14.3 Hz, 3H), 1.38 (d, J=14.8 Hz, 9H), 2.51-2.69 (m, 2H), 3.03 (dq, J=6.8, 6.8, 6.8, 18.6 Hz, 1H), 3.26 (ddq, J=6.9, 7.0, 7.0, 14.0, 28.9 Hz, 1H), 4.18 (ddd, J=5.1, 9.9, 114.9 Hz, 1H), 4.97-5.18 (m, 2H), 5.64-5.90 (m, 1H), 12.52 (s, 1H).

### Int. 42: Preparation of (S)-2-((tert-butoxycarbonyl)(methyl)amino)hex-5-enoic acid

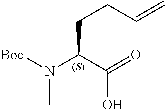

### Step 1: (S)-1-[(2R)-1-benzylpyrrolidine-2-carbonyl]-5-(but-3-en-1-yl)-7-phenyl-5H-benzo[d]1-oxa-3,7-diaza-2-nickelacyclononan-4-one

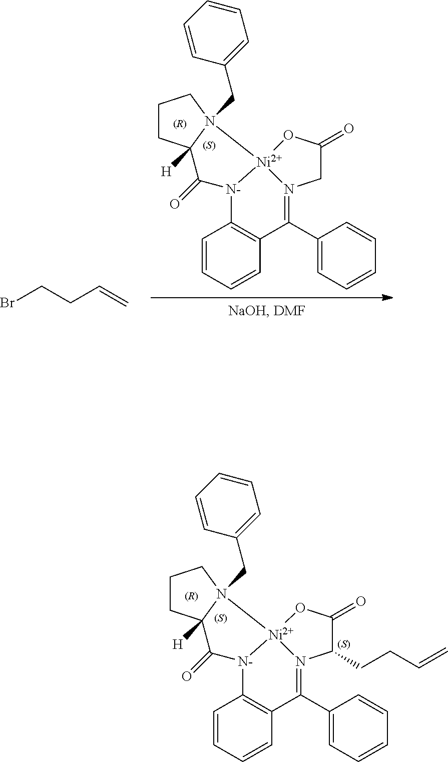

A solution of 1-[(2R)-1-benzylpyrrolidine-2-carbonyl]-7-phenyl-5H-benzo[d]1-oxa-3,7-diaza-2-nickelacyclononan-4-one (75.31 g, 151.17 mmol, 1 eq) and NaOH (60.46 g, 1511.67 mmol, 10 eq) in DMF (600 mL) was stirred for 15 min at rt under nitrogen atmosphere. 4-bromo-1-butene (20 g, 148.14 mmol, 0.98 eq) was added to the solution dropwise and stirred for 1.5 h at rt. The mixture was acidified to pH=6 with HOAc (5%) and extracted with EtOAc (3×1 L). The combined organic layers were washed with NH₄Cl (aq.) (3×1 L) and dried over anhydrous Na₂SO₄. After filtration, the filtrate was concentrated under vacuum and purified by silica gel column chromatography, eluted with CH₂Cl₂/MeOH (20:1) to afford crude product (5S)-1-[(2R)-1-benzylpyrrolidine-2-carbonyl]-5-(but-3-en-1-yl)-7-phenyl-5H-benzo[d]1-oxa-3,7-diaza-2-nickelacyclononan-4-one (80 g, 86%) as a red solid. LCMS (ESI+): m/z 552.3 (M+H⁺). ¹H NMR (400 MHz, DMSO-d₆): δ 1.19 (t, J=7.1 Hz, 7H), 1.59 (dtd, J=13.3, 6.6, 3.3 Hz, 1H), 1.99 (s, 5H), 2.07 (s, 6H), 2.02-2.25 (m, 3H), 2.48 (s, 1H), 3.33 (s, 4H), 3.34-3.48 (m, 1H), 3.48-3.62 (m, 2H), 3.64 (td, J=10.6, 9.7, 3.7 Hz, 1H), 3.99-4.13 (m, 5H), 4.82 (dd, J=10.2, 1.9 Hz, 1H), 4.85-4.97 (m, 1H), 5.49 (s, 1H), 5.95 (s, 1H), 6.55 (dd, J=8.2, 1.7 Hz, 1H), 6.61-6.74 (m, 1H), 7.03-7.16 (m, 2H), 7.13-7.27 (m, 1H), 7.36 (t, J=7.7 Hz, 2H), 7.39-7.49 (m, 1H), 7.45-7.53 (m, 1H), 7.50-7.64 (m, 3H), 7.98 (dd, J=8.7, 1.2 Hz, 1H), 8.32-8.43 (m, 2H).

### Step 2: (2S)-2-aminohex-5-enoic acid

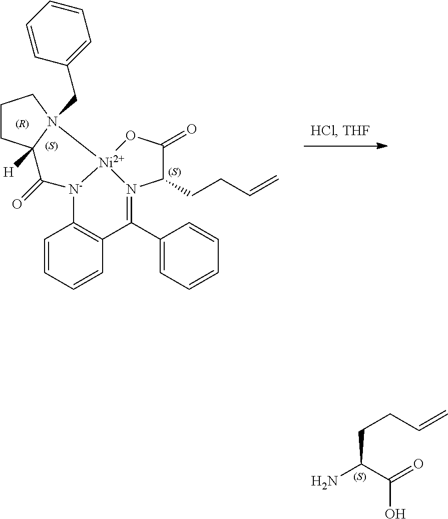

A solution of (5S)-1-[(2R)-1-benzylpyrrolidine-2-carbonyl]-5-(but-3-en-1-yl)-7-phenyl-5H-benzo[d]1-oxa-3,7-diaza-2-nickelacyclononan-4-one (100 g, 181.06 mmol, 1 eq) and HCl (600 mL, 3N) in THF (600 mL) was stirred for 2 h at 80° C. The mixture was basified to pH=7 with NaOH/Na₂CO₃. The resulting mixture was extracted with CH₂Cl₂ (3×500 mL). (2S)-2-aminohex-5-enoic acid (25 g, 427%) was in water layer was used in the next step directly without further purification. LCMS (ESI+): m/z 130.15 (M+H⁺).

### Step 3: (2S)-2-[(tert-butoxycarbonyl)amino]hex-5-enoic acid

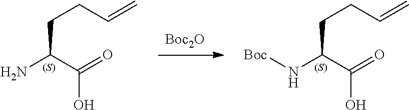

To the above water solution (1.5 L) including (2S)-2-aminohex-5-enoic acid (25 g, 193.56 mmol, 1 eq) was added Boc₂O (84.50 g, 387.12 mmol, 2 eq) was stirred for 16 h at rt. The mixture was acidified to pH=6 with citric acid and extracted with CH₂Cl₂ (3×500 mL). The combined organic layers were washed with brine (1×200 mL) and dried over anhydrous Na₂SO₄. After filtration, the filtrate was concentrated under vacuum. The residue was purified by reversed-phase flash chromatography with the following conditions: column, C18 silica gel; mobile phase, Acetonitrile in Water, 5% to 100% gradient in 30 min; detector, UV 210 nm. This resulted in (2S)-2-[(tert-butoxycarbonyl)amino]hex-5-enoic acid (12 g, 135%) as a light-yellow oil. LCMS (ESI+): m/z 130.15 (M+H⁺). ¹H NMR (400 MHz, DMSO-d₆): δ 1.18 (t, J=7.1 Hz, 1H), 1.35 (s, 1H), 1.39 (s, 8H), 1.57-1.69 (m, 1H), 1.66-1.78 (m, 1H), 1.91 (s, 2H), 1.99 (s, 1H), 2.06 (dq, J=13.9, 7.2 Hz, 2H), 3.88 (ddd, J=9.5, 8.1, 4.7 Hz, 1H), 4.03 (q, J=7.1 Hz, 1H), 4.94-5.06 (m, 2H), 5.78 (ddt, J=16.9, 10.2, 6.6 Hz, 1H), 7.08 (d, J=8.1 Hz, 1H), 12.26 (s, 2H).

### Step 4: methyl(S)-2-((tert-butoxycarbonyl)(methyl)amino)hex-5-enoate

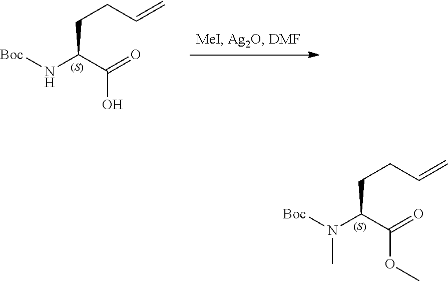

To a stirred solution of (2S)-2-[(tert-butoxycarbonyl)amino]hex-5-enoic acid (6 g, 26.17 mmol, 1 eq) and Ag₂O (24.26 g, 104.68 mmol, 4 eq) in DMF (90 mL) was added CH₃I (55.72 g, 392.54 mmol, 15 eq) dropwise at 0° C. The resulting mixture was stirred overnight at rt. The resulting mixture was filtered, the filter cake was washed with DMF (3×50 mL). The filtrate was used in the next step directly without further purification. LCMS (ESI+): m/z 258.20 (M+H⁺).

### Step 5: Int. 42

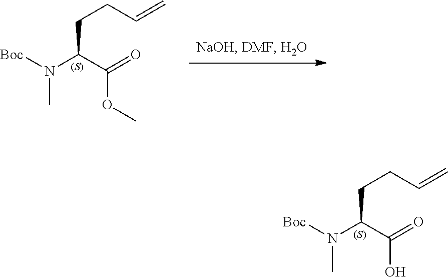

To a stirred solution of methyl (2S)-2-[(tert-butoxycarbonyl)(methyl)amino]hex-5-enoate (20 g, 77.72 mmol, 1 eq) in H₂O (500 mL)/DMF (500 mL) was added and NaOH (18.65 g, 466.33 mmol, 6 eq) in portions at 0° C. The resulting mixture was stirred for 16 h at rt and acidified to pH 6 with HCl (3N). The mixture was extracted with CH₂Cl₂ (3×0.5 L). The combined organic layers were washed with brine (3×300 mL) and dried over anhydrous Na₂SO₄. After filtration, the filtrate was concentrated under vacuum and purified by reversed-phase flash chromatography with the following conditions: column, C18 silica gel; mobile phase, Acetonitrile in Water, 5% to 50% gradient in 30 min; detector, UV 200 nm. This resulted in racemic product (20 g) which was purified by Prep-CHIRAL-HPLC with the following conditions (Column: CHIRALPAK IC-3, 4.6×50 mm, 3 m; Mobile Phase A: Hex (0.1% TFA): IPA=98:2; Flow rate: 1 mL/min; Gradient: 0% B to 0% B; Injection Volume: 5 ul mL) to afford Int. 42 (15.78 g, 83%) as a light yellow oil. LCMS (ESI+): m/z 242.10 (M+H⁺). ¹H NMR (300 MHz, DMSO-d₆) δ 1.38 (d, J=13.4 Hz, 17H), 1.71-1.86 (m, 1H), 1.89 (s, 1H), 1.94 (d, J=4.9 Hz, 1H), 1.94-2.12 (m, 3H), 2.72 (s, 6H), 4.26 (dd, J=10.4, 4.6 Hz, 1H), 4.50 (dd, J=10.8, 4.4 Hz, 1H), 4.93-5.05 (m, 3H), 5.02-5.10 (m, 1H), 5.81 (ddd, J=16.9, 10.2, 3.6 Hz, 1H), 12.73 (s, 2H).

### Int. 43: Preparation of (S)-2-((tert-butoxycarbonyl)(methyl)amino)hept-6-enoic acid

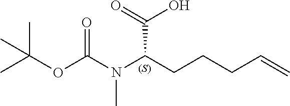

### Step 1: (5S)-1-[(2S)-1-benzylpyrrolidine-2-carbonyl]-5-(pent-4-en-1-yl)-7-phenyl-5H-benzo[d]1-oxa-3,7-diaza-2-nickelacyclononan-4-one

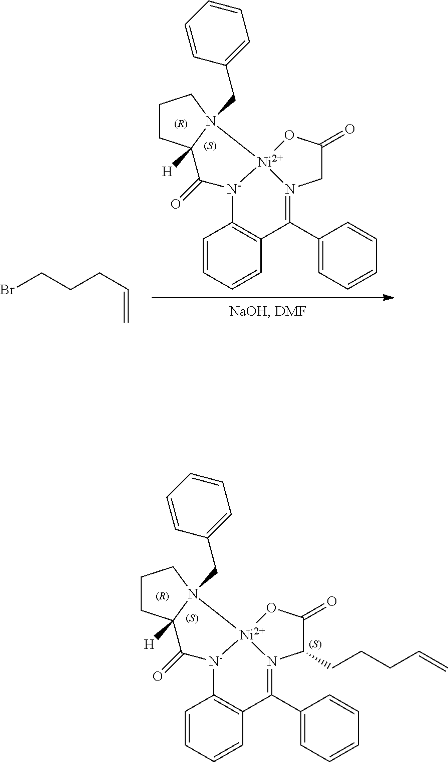

To a solution of NaOH (80.51 g, 2013.0 mmol, 10 eq.) in DMF (270 mL), 1-[(2S)-1-benzylpyrrolidine-2-carbonyl]-7-phenyl-1H,4H,5H-benzo[d]1-oxa-3,7-diaza-2-nickelacyclononan-4-one (102.29 g, 205.33 mmol, 1.02 eq) was added in at 25° C. under nitrogen atmosphere. The reaction was stirred for 15 min at 25° C. 5-bromopent-1-ene (30 g, 201.30 mmol, 1 eq) was dropwise below 25° C. The reaction was stirred for 5 hours at 25° C. The reaction was purified together with Page EB2141275-183 and EB2141275-184. The mixture was acidified to pH=3 with acetic acid (5%), The resulting mixture was extracted with EtOAc (2×250 mL). The combined organic layers were dried over anhydrous Na₂SO₄. After filtration, the filtrate was concentrated under reduced pressure. The residue was purified by silica gel column chromatography, eluted with CH2Cl2/MeOH (20:1) to afford (5S)-1-[(2S)-1-benzylpyrrolidine-2-carbonyl]-5-(pent-4-en-1-yl)-7-phenyl-5H-benzo[d]1-oxa-3,7-diaza-2-nickelacyclononan-4-one (88 g, 77%) as a yellow oil. LCMS (ESI+): m/z 566 (M+H⁺). ¹H NMR (400 MHz, DMSO-d₆): δ 1.54 (dddd, 2H), 1.76-2.04 (m, 3H), 2.12-2.34 (m, 3H), 2.45 (t, 2H), 3.32 (d, 2H), 3.50-3.71 (m, 3H), 4.07 (d, 1H), 4.85-5.09 (m, 2H), 5.76 (ddt, 1H), 6.53 (dd, 1H), 6.66 (ddd, 1H), 7.01-7.24 (m, 3H), 7.36 (t, 2H), 7.45-7.66 (m, 4H), 7.91-8.09 (m, 1H), 8.27-8.46 (m, 2H).

### Step 2: (S)-2-aminohept-6-enoic acid

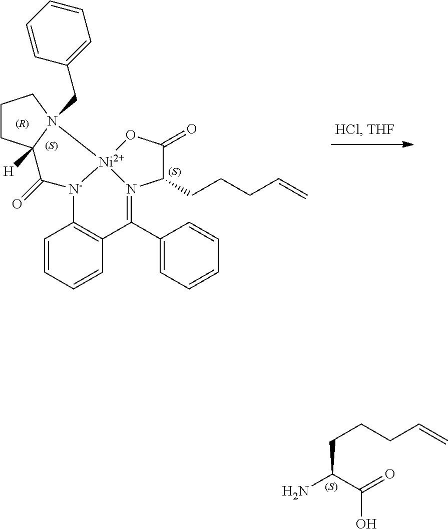

To a solution of (5S)-1-[(2S)-1-benzylpyrrolidine-2-carbonyl]-5-(pent-4-en-1-yl)-7-phenyl-5H-benzo[d]1-oxa-3,7-diaza-2-nickelacyclononan-4-one (88 g, 155.39 mmol, 1 eq.) in THF (880.0 g, 12204.1 mmol, 78.54 eq) was added HCl (517.96 mL, 1553.87 mmol, 10 eq) at 25° C. The reaction was stirred for 2 hours at 60° C. The resulting mixture was diluted with water (1500 mL). The mixture was basified to pH=8 with NaOH (aq., 2N). The resulting mixture was extracted with EtOAc (2×500 mL). The aqueous phase (including about 22 g product) was used in next step without further purification. LCMS (ESI+): m/z 144 (M+H⁺).

### Step 3: (2S)-2-[(tert-butoxycarbonyl)amino]hept-6-enoic acid

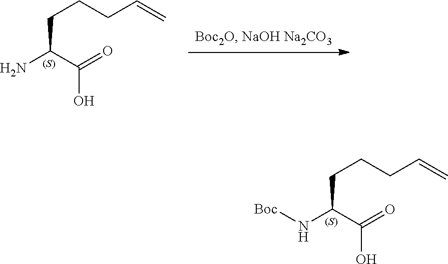

To a solution of (2S)-2-aminohept-6-enoic acid (22 g, 153.65 mmol, 1 eq), Na₂CO₃ (97.71 g, 921.88 mmol, 6 eq) in water (2000 mL) was added Boc₂O (100.60 g, 460.94 mmol, 3 eq) at 25° C. The reaction was stirred for 6 hours at 25° C. The mixture was acidified to pH=6 with diluted citric acid. The resulting mixture was extracted with EtOAc (3×150 mL). The combined organic layers were dried over anhydrous Na₂SO₄. After filtration, the filtrate was concentrated under reduced pressure. The residue was purified by silica gel column chromatography, eluted with CH₂Cl₂/MeOH (20:1) to afford (2S)-2-[(tert-butoxycarbonyl)amino]hept-6-enoic acid (30 g, 80%) as a yellow oil. LCMS (ESI+): m/z 266 (M+Na⁺). ¹H NMR (400 MHz, DMSO-d₆): δ 1.38 (s, 11H), 1.50-1.59 (m, 1H), 1.66 (dddd, 1H), 2.74 (s, 1H), 2.90 (s, 1H), 3.86 (ddd, 1H), 4.95 (ddt, 1H), 5.00 (dt, 1H), 5.68-5.88 (m, 1H), 7.07 (d, 1H), 12.38 (s, 1H).

### Step 4: Methyl(S)-2-((tert-butoxycarbonyl)(methyl)amino)hept-6-enoate

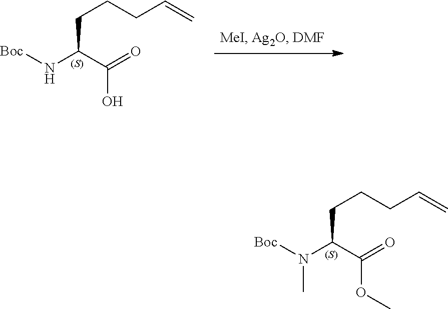

MeI (175.02 g, 1233.03 mmol, 10 eq) was added into a solution of (2S)-2-[(tert-butoxycarbonyl)amino]hept-6-enoic acid (30 g, 123.30 mmol, 1 eq) and Ag₂O (142.87 g, 616.52 mmol, 5 eq) in DMF (200 mL). The reaction was stirred for 6 hours at 25° C. Desired product could be detected by LCMS. The resulting mixture was filtered, the filter cake was washed with DMF (2×5 mL). The filtrate was used in next step without further purification (about 35 g). LCMS (ESI+): m/z 172 (M+H−Boc⁺).

### Step 5: Int. 43

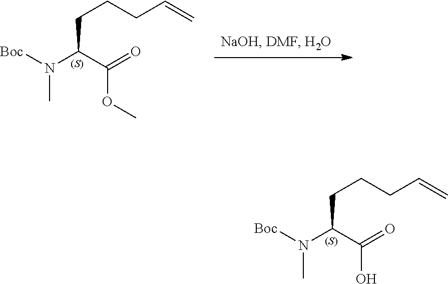

NaOH (25.79 g, 644.91 mmol, 5 eq) in water (350.00 mL, 19428.41 mmol, 150.63 eq) was added into a solution of methyl (2S)-2-[(tert-butoxycarbonyl)(methyl)amino]hept-6-enoate (35 g, 128.98 mmol, 1 eq) in DMF (20 mL) at 25° C. The reaction was stirred for 5 hours at 25° C. Desired product could be detected by LCMS. The reaction was purified with Page EB2206983-001 and EB2206983-002 and EB2206983-003 and EB2206983-004. The resulting mixture was diluted with water (500 mL). The mixture was acidified to pH=6 with HCl (aq. 2N). The resulting mixture was extracted with EtOAc (3×1000 mL). The combined organic layers were washed with saturated ammonium chloride (2×500 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 10% to 60% gradient in 20 min; detector, UV 254 nm to afford (2S)-2-[(tert-butoxycarbonyl)(methyl)amino]hept-6-enoic acid (31 g, 93.40%) as a light yellow oil. The compound was further purified by PREP_CHIRAL_HPLC with the following conditions: Column: CHIRAL ART Cellulose-SC, 2*25 cm, 5 m; Mobile Phase A: Hex (0.1% FA)—HPLC, Mobile Phase B: IPA—HPLC; Flow rate: 20 mL/min; Gradient: 2% B to 2% B in 17 min; Wave Length: 220/254 nm; RT1 (min): 6.939; RT2 (min): 12.248; Sample Solvent: EtOH—HPLC to afford Int. 43 (21.4 g, 66%) as a light yellow oil. LCMS (ESI+): m/z 280 (M+Na⁺). ¹H NMR (400 MHz, DMSO-d₆): δ 1.39 (d, 11H), 1.56-1.88 (m, 2H), 2.05 (qp, 2H), 2.70 (s, 3H), 4.41 (ddd, 1H), 4.92-5.09 (m, 2H), 5.71-5.88 (m, 1H), 12.65 (s, 1H).

### Int. 44: 1-(methylsulfonyl)-3-(4,4,5,5-tetramethyl-1,3,2-dioxaborolan-2-yl)-2,5-dihydro-1H-pyrrole

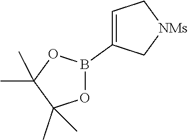

This material was synthesized according to the procedure for Int. 40 with tert-butyl 3-(4,4,5,5-tetramethyl-1,3,2-dioxaborolan-2-yl)-2,5-dihydro-1H-pyrrole-1-carboxylate and methanesulfonyl chloride as starting materials. LCMS (ESI+): m/z 274.2 (M+H⁺).

### Int. 45: 1-(3-(4,4,5,5-tetramethyl-1,3,2-dioxaborolan-2-yl)-8-azabicyclo[3.2.1]oct-2-en-8-yl)ethan-1-one

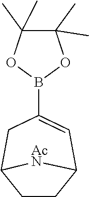

This material was synthesized according to the procedure for Int. 40 with tert-butyl 3-(4,4,5,5-tetramethyl-1,3,2-dioxaborolan-2-yl)-8-azabicyclo[3.2.1]oct-2-ene-8-carboxylate as the starting material LCMS (ESI+): m/z 278.2 (M+H⁺).

### Int. 122: 1-(3,3-dimethyl-4-(4,4,5,5-tetramethyl-1,3,2-dioxaborolan-2-yl)-3,6-dihydropyridin-1 (2H)-yl)ethan-1-one

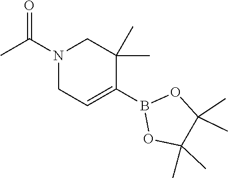 [SMILES: CC(=O)N1CC=C(B2OC(C)(C)C(C)(C)O2)C(C)(C)C1]

Int. 122 was synthesized according to the procedure Int. 40 with tert-butyl 3,3-dimethyl-4-(4,4,5,5-tetramethyl-1,3,2-dioxaborolan-2-yl)-3,6-dihydropyridine-1 (2H)-carboxylate as starting material. LCMS (ESI+): m/z 280.5 (M+H⁺).

### Int. 127: (S)-2-cyclopropyl-2-((2S,4R)-1-(3,3-difluoro-1-(trifluoromethyl)cyclobutene-1-carbonyl)-4-fluoropyrrolidine-2-carboxamido)acetic acid

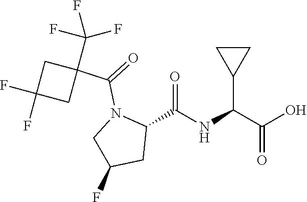 [SMILES: O=C(O)[C@@H](NC(=O)[C@@H]1C[C@@H](F)CN1C(=O)C1(C(F)(F)F)CC(F)(F)C1)C1CC1]

This material was synthesized in an identical way to Int. 34 using commercially available FMOC protected amino acids. LCMS (ESI+): m/z 417.1 (M+H⁺).

### Int. 129: (S)-2-cyclopropyl-2-((2S,4R)-4-fluoro-1-(1-(trifluoromethyl)cyclopropane-1-carbonyl)pyrrolidine-2-carboxamido)acetic acid

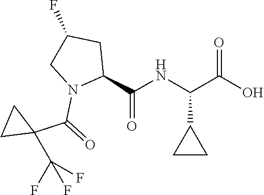 [SMILES: O=C(O)[C@@H](NC(=O)[C@@H]1C[C@@H](F)CN1C(=O)C1(C(F)(F)F)CC1)C1CC1]

This material was synthesized in an identical way to Int. 34 using commercially available FMOC protected amino acids. LCMS (ESI+): m/z 367.4 (M+H⁺).

### Int. 140: Preparation of (2S,4R)-4-fluoro-1-(1-(trifluoromethyl)cyclopropane-1-carbonyl)pyrrolidine-2-carboxylic acid

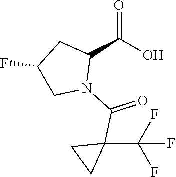

This compound was prepared following the general synthetic sequence described for the preparation of Int. 141 With 1-(trifluoromethyl)cyclopropane-1-carboxylic acid as the starting material. LCMS (ESI+): m/z 417.99 (M+H⁺).

### Int. 141: Preparation of (2S,4R)-1-(3,3-difluoro-1-(trifluoromethyl)cyclobutane-1-carbonyl)-4-fluoropyrrolidine-2-carboxylic acid

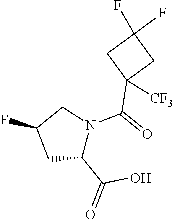 [SMILES: O=C(O)[C@@H]1C[C@@H](F)CN1C(=O)C1(C(F)(F)F)CC(F)(F)C1]

### Step 1: Methyl (2S,4R)-4-fluoropyrrolidine-2-carboxylate

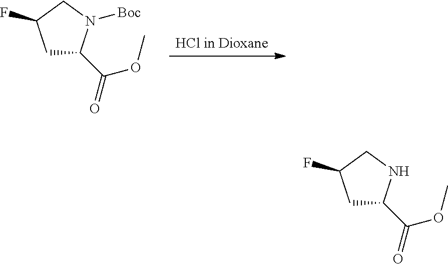

A solution of 1-tert-butyl 2-methyl (2S,4R)-4-fluoropyrrolidine-1,2-dicarboxylate (150 g, 101.1 mmol, 1 eq) and HCl_{(gas)} in 1,4-dioxane (758.29 mL, 505.53 mmol, 5 eq) was stirred for 0.5 h at rt and then concentrated under vacuum to afford methyl (2S,4R)-4-fluoropyrrolidine-2-carboxylate (110 g, yield, 99%, purity 80%) as a white solid. The crude product was used in the next step directly without further purification. LCMS (ESI+): m/z 148.15 (M+H⁺).

### Step 2: Methyl (2S,4R)-1-[3,3-difluoro-1-(trifluoromethyl)cyclobutanecarbonyl]-4-fluoropyrrolidine-2-carboxylate

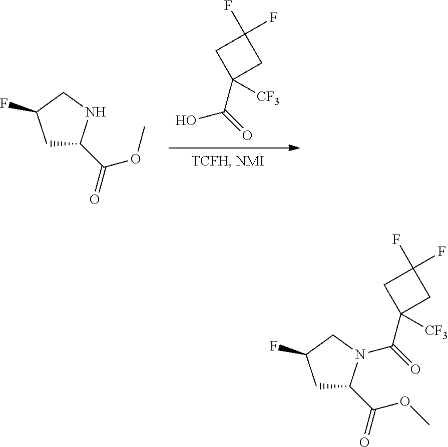

To a stirred solution of methyl (2S,4R)-4-fluoropyrrolidine-2-carboxylate (110 g, 103.3 mmol, 1 eq, 80%), 3,3-difluoro-1-(trifluoromethyl)cyclobutane-1-carboxylic acid (122.06 g, 103.3 mmol, 1 eq) and TCFH (251.69 g, 154.95 mmol, 1.5 eq) in ACN (1100 mL) was added NMI (245.51 g, 516.49 mmol, 5 eq) dropwise in 45 min at 0° C. The mixture was slowly warmed up to rt and stirred overnight at rt. The mixture was concentrated under vacuum at 28° C. and diluted with EtOAc (900 mL) and washed with HCl (0.5N, 1400 mL×1). The aqueous layer was extracted again with EtOAc (1×500 mL). The combined organic layers were washed with saturated NaHCO₃ (1000 mL×1). The aqueous layer was extracted with EtOAc (300 mL×1) again. The combined organic layers were washed with brine and dried over anhydrous Na₂SO₄. After filtration, the filtrate was concentrated under vacuum to afford methyl (2S,4R)-1-[3,3-difluoro-1-(trifluoromethyl)cyclobutanecarbonyl]-4-fluoropyrrolidine-2-carboxylate 180 g, 81%) as a light-yellow solid. The crude product was used in the next step directly without further purification. LCMS (ESI+): m/z 334.15 (M+H⁺).

### Step 3: Int. 141

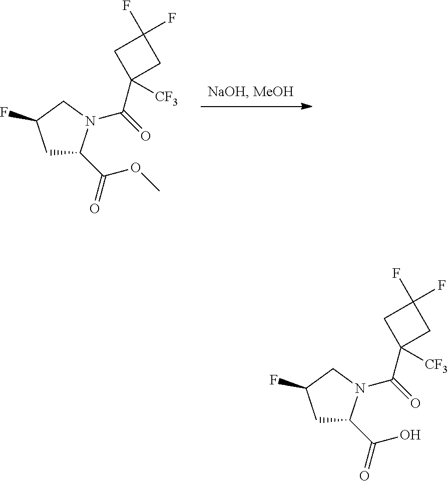

To a stirred solution of methyl (2S,4R)-1-[3,3-difluoro-1-(trifluoromethyl)cyclobutanecarbonyl]-4-fluoropyrrolidine-2-carboxylate (180 g, 82.15 mmol, 1 eq, 90%) in MeOH (1400 mL) was added dropwise NaOH (58.33 g, 246.45 mmol, 3 eq) in H₂O (400 mL) in 30 min at 0-20° C. The mixture was stirred for 2 h at rt. MeOH was evaporated out under vacuum. The residue was diluted with water (2500 mL) and acidified with HCl (3N, 400 mL) at 0-20° C. Then the precipitated solids were collected by filtration and washed with water (3×200 mL). The filtrate was added HCl (3N, 100 mL). The precipitated solids were collected by filtration and washed with water (3×100 mL). The combined solids were dried in an oven for 16 h at 40° C. This resulted in afford Int. 40 (150.7 g, 97%) as a white solid. LCMS (ESI+): m/z 319.95 (M+H⁺).

### Int. 142: Preparation of (2S,4R)-4-fluoro-1-((R)-3,3,3-trifluoro-2-hydroxy-2-methylpropanoyl)pyrrolidine-2-carboxylic acid

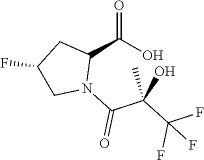

This compound was prepared following the general synthetic sequence described for the preparation of Int. 141. With (R)-3,3,3-trifluoro-2-hydroxy-2-methylpropanoic acid as the starting material. LCMS (ESI+): m/z 421.08

### Int. 143: Preparation of (2S,4R)-4-fluoro-1-(2-(trifluoromethyl)spiro[3.3]heptane-2-carbonyl)pyrrolidine-2-carboxylic acid

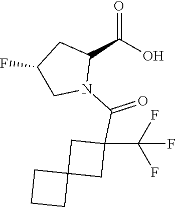

This compound was prepared following the general synthetic sequence described for the preparation of Int. 141 with 2-(trifluoromethyl)spiro[3.3]heptane-2-carboxylic acid as the starting material. LCMS (ESI+): m/z 323.11 (M+H⁺).

### Int. 144: Preparation of (2S,4R)-4-fluoro-1-(1-(trifluoromethyl)cyclobutane-1-carbonyl)pyrrolidine-2-carboxylic acid

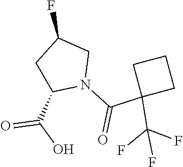 [SMILES: O=C(O)[C@@H]1C[C@@H](F)CN1C(=O)C1(C(F)(F)F)CCC1]

This compound was prepared following the general synthetic sequence described for the preparation of Int. 141 with 1-(trifluoromethyl)cyclobutane-1-carboxylic acid as the starting material. LCMS (ESI+): m/z 283.08 (M+H⁺).

### Int. 145: Preparation of (1-(trifluoromethyl)cyclobutane-1-carbonyl)-L-proline

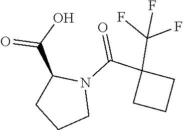 [SMILES: O=C(O)[C@@H]1CCCN1C(=O)C1(C(F)(F)F)CCC1]

This compound was prepared following the general synthetic sequence described for the preparation of Int. 141 with (tert-butoxycarbonyl)-L-proline and 1-(trifluoromethyl)cyclobutane-1-carboxylic acid as the starting materials. LCMS (ESI+): m/z 266.1 (M+H⁺).

### Int. 146: (3,3-difluoro-1-(trifluoromethyl)cyclobutane-1-carbonyl)-L-proline

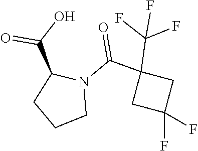 [SMILES: O=C(O)[C@@H]1CCCN1C(=O)C1(C(F)(F)F)CC(F)(F)C1]

This compound was prepared following the general synthetic sequence described for the preparation of Int. 141 with (tert-butoxycarbonyl)-L-proline as the starting material. LCMS (ESI+): m/z 302.2 (M+H⁺).

### Int. 147: Preparation of (2S,4R)-1-(1-cyanocyclobutane-1-carbonyl)-4-fluoropyrrolidine-2-carboxylic acid

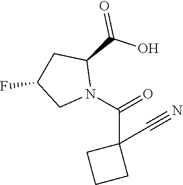 [SMILES: N#CC1(C(=O)N2C[C@H](F)C[C@H]2C(=O)O)CCC1]

This compound was prepared following the general synthetic sequence described for the preparation of Int. 141 with 1-cyanocyclobutane-1-carboxylic acid as the starting material. LCMS (ESI+): m/z 240.09 (M+H⁺).

### Int. 148 and 149: Preparation of (2S,4R)-4-fluoro-1-((R)-2-(trifluoromethyl)tetrahydro-2H-pyran-2-carbonyl)pyrrolidine-2-carboxylic acid and (2S,4R)-4-fluoro-1-((S)-2-(trifluoromethyl)tetrahydro-2H-pyran-2-carbonyl)pyrrolidine-2-carboxylic acid

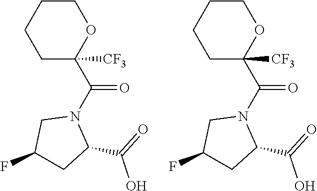

### Step 1: Methyl (2S,4R)-4-fluoropyrrolidine-2-carboxylate

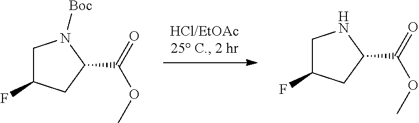

A mixture of 1-(tert-butyl) 2-methyl (2S,4R)-4-fluoropyrrolidine-1,2-dicarboxylate (20 g, 80.9 mmol, 1 eq) in HCl/EtOAc (200 mL) (4 M) was stirred at 25° C. for 2 h. LC-MS showed starting material was consumed and one main peak with desired mass was detected. The reaction mixture was concentrated to give methyl (2S,4R)-4-fluoropyrrolidine-2-carboxylate (15 g, crude, HCl) as a light-yellow solid. This was taken on to the next reaction without further purification.

### Step 2: (2S,4R)-4-fluoro-1-((R)-2-(trifluoromethyl)tetrahydro-2H-pyran-2-carbonyl)pyrrolidine-2-carboxylate and (2S,4R)-4-fluoro-1-((S)-2-(trifluoromethyl)tetrahydro-2H-pyran-2-carbonyl)pyrrolidine-2-carboxylate

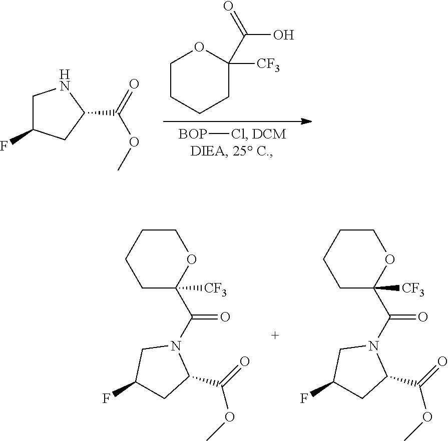

To a solution of 2-(trifluoromethyl)tetrahydro-2H-pyran-2-carboxylic acid (13 g, 65.61 mmol, 1 eq) in DCM (200 mL) was added DIEA (8.48 g, 65.61 mmol, 11.43 mL, 1 eq), and the mixture was stirred for 10 min, then BOP—Cl (18.37 g, 72.17 mmol, 1.1 eq), methyl (2S,4R)-4-fluoropyrrolidine-2-carboxylate, and DIEA (16.96 g, 131.22 mmol, 22.86 mL, 2 eq) were added. The mixture was stirred at 25° C. for 12 h. LC-MS showed one main peak with desired mass was detected. The reaction mixture was concentrated under reduced pressure to remove DCM, and then EtOAc (30 mL) was added. The solution was washed three times with 5% NaHCO₃ solution (15 mL) and once consecutively with water (15 mL), 2M HCl solution (15 mL), water (15 mL) and saturated brine (15 mL). The organic layer was dried over anhydrous Na₂SO₄, filtered and the filtrate was concentrated under reduced pressure to give a residue. The residue was purified by column chromatography (SiO₂, PE:EtOAc=10:1 to 2:1) to give methyl (2S,4R)-4-fluoro-1-((R)-2-(trifluoromethyl)tetrahydro-2H-pyran-2-carbonyl)pyrrolidine-2-carboxylate (10.7 g, 31.7 mmol, 48% yield, 96.8% purity) as a light yellow oil and to give methyl (2S,4R)-4-fluoro-1-((S)-2-(trifluoromethyl)tetrahydro-2H-pyran-2-carbonyl)pyrrolidine-2-carboxylate (11 g, 32.0 mmol, 49% yield, 95.2% purity) as a light yellow oil.

### Step 3: (2S,4R)-4-fluoro-1-((R)-2-(trifluoromethyl)tetrahydro-2H-pyran-2-carbonyl)pyrrolidine-2-carboxylic acid

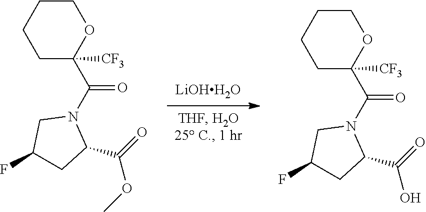 [SMILES: COC(=O)[C@@H]1C[C@@H](F)CN1C(=O)[C@@]1(C(F)(F)F)CCCCO1 ; "LiOH·H2O" ; "THF, H2O25° C., 1 hr" ; O=C(O)[C@@H]1C[C@@H](F)CN1C(=O)[C@@]1(C(F)(F)F)CCCCO1]

To a solution of methyl (2S,4R)-4-fluoro-1-((R)-2-(trifluoromethyl)tetrahydro-2H-pyran-2-carbonyl)pyrrolidine-2-carboxylate (10.7 g, 31.66 mmol, 96.83% purity, 1 eq) in THF (100 mL) and H₂O (100 mL) was added LiOH·H₂O (2.66 g, 63.32 mmol, 2 eq). Then the mixture was stirred at 25° C. for 1 hr. LC-MS showed methyl (2S,4R)-4-fluoro-1-((R)-2-(trifluoromethyl)tetrahydro-2H-pyran-2-carbonyl)pyrrolidine-2-carboxylate was consumed and one main peak with desired mass was detected. The mixture was acidified at 0° C. with HCl (1 N) until pH=2-3. Then the mixture was extracted with EtOAc 300 mL (100 mL×3). The combined organic layers were dried over Na₂SO₄, filtered, and concentrated under reduced pressure to give a residue. The residue was purified by column chromatography (SiO₂, PE:EtOAc=10:1 to 2:1) to give (2S,4R)-4-fluoro-1-((R)-2-(trifluoromethyl)tetrahydro-2H-pyran-2-carbonyl)pyrrolidine-2-carboxylic acid (8.9 g, 28.33 mmol, 89% yield, 99% purity) as a white solid. LCMS (ESI+): m/z 314.0 (M+H⁺).

### Step 4: Int. 149

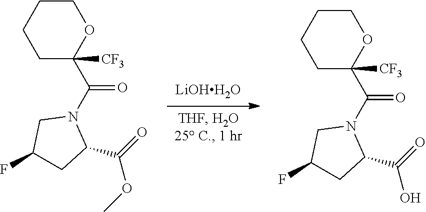 [SMILES: COC(=O)[C@@H]1C[C@@H](F)CN1C(=O)[C@]1(C(F)(F)F)CCCCO1 ; "LiOH·H2O" ; "THF, H2O25° C., 1 hr" ; O=C(O)[C@@H]1C[C@@H](F)CN1C(=O)[C@]1(C(F)(F)F)CCCCO1]

The same hydrolysis procedure was performed on methyl (2S,4R)-4-fluoro-1-((S)-2-(trifluoromethyl)tetrahydro-2H-pyran-2-carbonyl)pyrrolidine-2-carboxylate to provide the desired product Int. 149 (9.1 g, 28.97 mmol, 90.5% yield, 99.7% purity) as a white solid. LCMS (ESI+): m/z 314.0 (M+H⁺).

### Int. 150: Preparation of (2S,4R)-4-fluoro-1-(3,3,3-trifluoro-2,2-dimethylpropanoyl)pyrrolidine-2-carboxylic acid

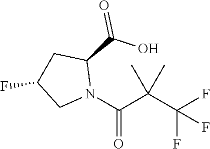 [SMILES: CC(C)(C(=O)N1C[C@H](F)C[C@H]1C(=O)O)C(F)(F)F]

Int. 150 was prepared following the general synthetic sequence described for the preparation of Int. 141. LCMS (ESI+): m/z 271.08 (M+H⁺).

### Int. 154: Preparation of tert-butyl(S)-2-((((9H-fluoren-9-yl)methoxy)carbonyl)(methyl)amino)pent-4-enoate

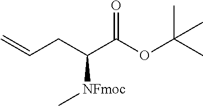

### Step 1: Int. 154

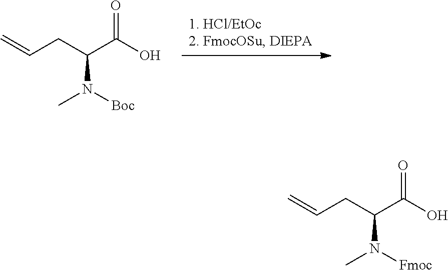

To a solution of Int. 152 (250 g, 1.09 mol, 1 eq) in HC/EtOAc (2500 mL). The mixture was stirred at 25° C. for 12 hour. The reaction mixture was concentrated under reduced pressure to remove solvent to give the deprotected HCl salt (180.5 g, crude, HCl) as a white solid. ¹H NMR (400 MHz, DMSO-d₆) δ 14.33-13.58 (m, 1H), 10.10-8.94 (m, 2H), 5.84-5.69 (m, 1H), 5.31-5.11 (m, 2H), 3.99 (br t, J=5.2 Hz, 1H), 2.68 (br t, J=6.4 Hz, 2H), 2.60-2.52 (m, 3H).

To a solution of the crude HCl salt (170 g, 1.03 mol, 1 eq) in H₂O (860 mL) and dioxane (1140 mL) was added FmocOSu (346 g, 1.03 mol, 1 eq) and DIEA (398 g, 3.08 mol, 536 mL, 3 eq). The mixture was stirred until the LCMS showed consumption of starting material. The reaction was quenched with citric acid solution (800 mL) at 25° C., and then extracted with EtOAc (500 mL×4). The combined organic layers were washed with brine (500 mL×2), dried over anhydrous Na₂SO₄, filtered and concentrated under reduced pressure to give a residue. The residue was purified by flash silica gel chromatography (Eluent of 0-40% EtOAc/PE) to give (S)-2-((((9H-fluoren-9-yl)methoxy)carbonyl)(methyl)amino)pent-4-enoic acid (290 g, 815 mmol, 80% yield, 99% purity) as a white solid. ¹H NMR (400 MHz, DMSO-d₆) δ 13.13-12.72 (m, 1H), 7.89 (dd, J=3.7, 7.2 Hz, 2H), 7.63 (br d, J=7.6 Hz, 2H), 7.45-7.27 (m, 4H), 5.73-5.46 (m, 1H), 5.17-4.95 (m, 2H), 4.67-4.44 (m, 1H), 4.40-4.21 (m, 3H), 2.80-2.70 (m, 3H), 2.53 (br s, 1H), 2.48-2.32 (m, 1H).

### Step 2: Int. 154

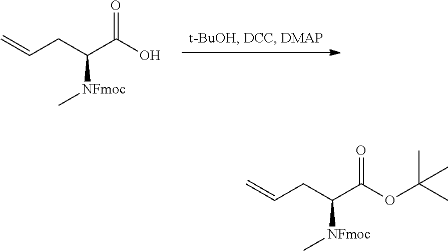

Dissolve (S)-2-((((9H-fluoren-9-yl)methoxy)carbonyl)(methyl)amino)pent-4-enoic acid (10 g, 1 Eq, 28 mmol) in DCM (20 mL), bring to 0° C. under argon. Add DCC (6.5 g, 1.1 Eq, 31 mmol) and DMAP (0.35 g, 0.1 Eq, 2.8 mmol) as solids. Add tert-butanol (8.4 g, 11 mL, 4 Eq, 0.11 mol) by syringe. Stir at rt overnight. Dilute in water and DCM, extract 3×DCM. Wash combined organic layers with water and brine, dry over MgSO₄, filter, concentrate. Purify by normal phase chromatography (0 to 100% EtOAc in hexanes) to yield Int. 154 (9.8 g, 24 mmol, 85%) as a pale oil.

### Int. 162: Preparation of (S)-1-(3,3-difluoro-1-(trifluoromethyl)cyclobutane-1-carbonyl)-4,4-difluoropyrrolidine-2-carboxylic acid

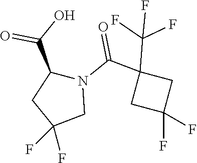 [SMILES: O=C(O)[C@@H]1CC(F)(F)CN1C(=O)C1(C(F)(F)F)CC(F)(F)C1]

### Step 1: 2-benzyl 1-(tert-butyl) (S)-4,4-difluoropyrrolidine-1,2-dicarboxylate

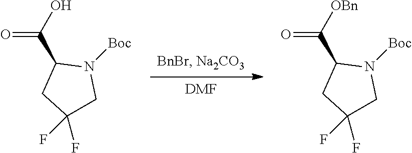

To a solution of (S)-1-(tert-butoxycarbonyl)-4,4-difluoropyrrolidine-2-carboxylic acid (2.50 g, 9.95 mmol, 1.0 Equiv.) in DMF (50 mL) was added Na₂CO₃ (2.11 g, 19.9 mmol, 2.0 Equiv.) and stirred at room temperature for 0.5 hrs. The reaction mixture was then cooled to 0° C. and BnBr (1.87 g, 10.9 mmol, 1.30 mL, 1.1 Equiv.) was added. The mixture was stirred at 0° C. for another 2.5 hrs. Water (80 mL) was added, and the aqueous phase was extracted with EtOAc (30 mL×3), the combined organic layer was washed by brine (30 mL), dried over Na₂SO₄, filtered and the filtrate was concentrated to give a residue. The residue was purified by silica gel column chromatography (Pet. Ether/EtOAc, 30:1) to afford 2-benzyl 1-(tert-butyl) (S)-4,4-difluoropyrrolidine-1,2-dicarboxylate (2.80 g, 8.20 mmol, 82.4% yield) as a clear oil. ¹H NMR (400 MHz, DMSO-d₆): δ7.37 (d, J=4.8 Hz, 5H), 5.23-5.10 (m, 2H), 4.59-4.50 (m, 1H), 3.83-3.70 (m, 2H), 3.02-2.87 (m, 1H), 2.59-2.50 (m, 1H), 1.41-1.25 (m, 9H).

### Step 2: benzyl(S)-4,4-difluoropyrrolidine-2-carboxylate

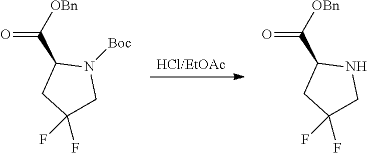 [SMILES: CC(C)(C)OC(=O)N1CC(F)(F)C[C@H]1C(=O)OCc1ccccc1 ; "HCl/EtOAc" ; O=C(OCc1ccccc1)[C@@H]1CC(F)(F)CN1]

To a solution of 2-benzyl 1-(tert-butyl) (S)-4,4-difluoropyrrolidine-1,2-dicarboxylate (2.00 g, 5.86 mmol, 1.0 Equiv.) in EtOAc (10 mL) was added HCl/EtOAc (4 M, 40 mL, 27.3 Equiv.) and stirred at 20° C. for 1 hr. The reaction mixture was concentrated to afford benzyl(S)-4,4-difluoropyrrolidine-2-carboxylate (2.14 g, crude) as a white solid. ¹H NMR (400 MHz, DMSO-d₆): δ10.78-10.59 (m, 1H), 7.48-7.33 (m, 5H), 5.30-5.22 (m, 2H), 4.86 (t, J=8.8 Hz, 1H), 3.83-3.64 (m, 2H), 2.97-2.85 (m, 1H), 2.75 (dq, J=8.8, 14.8 Hz, 1H).

### Step 3: benzyl(S)-1-(3,3-difluoro-1-(trifluoromethyl)cyclobutane-1-carbonyl)-4,4-difluoropyrrolidine-2-carboxylate

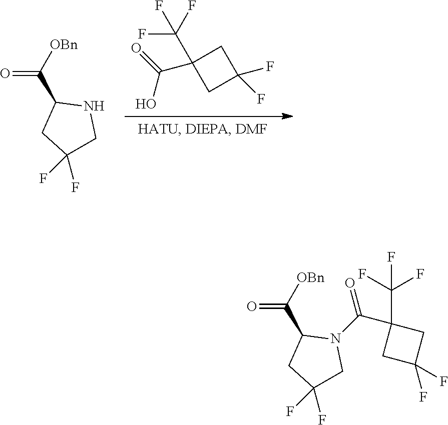

To a solution of benzyl(S)-4,4-difluoropyrrolidine-2-carboxylate (1.90 g, 7.88 mmol, 1.0 Equiv.) in DMF (30 mL) was added DIPEA (3.05 g, 23.6 mmol, 4.12 mL, 3.0 Equiv.), 3,3-difluoro-1-(trifluoromethyl)cyclobutanecarboxylic acid (2.41 g, 11.8 mmol, 1.5 Equiv.) and HATU (4.49 g, 11.8 mmol, 1.5 Equiv.), the mixture was stirred at 20° C. for 2 hrs. Water (60 mL) was added and the aqueous phase was extracted with EtOAc (20 mL×3). Then the organic layers was washed with brine (30 mL), dried over Na₂SO₄, filtered and the filtrate was concentrated to give a residue. The residue was purified by silica gel column chromatography, eluted with Pet. Ether/EtOAc (10:1 to 3:1) to afford benzyl(S)-1-(3,3-difluoro-1-(trifluoromethyl)cyclobutane-1-carbonyl)-4,4-difluoropyrrolidine-2-carboxylate (2.30 g, 5.38 mmol, 68.3% yield) as a yellow oil. ¹H NMR (400 MHz, DMSO-d₆): δ7.41-7.32 (m, 5H), 5.15 (s, 2H), 4.83 (dd, J=4.4, 10.0 Hz, 1H), 4.13-4.00 (m, 2H), 3.34-3.20 (m, 4H), 2.99-2.83 (m, 1H), 2.59-2.51 (m, 1H).

### Step 4: Int. 162

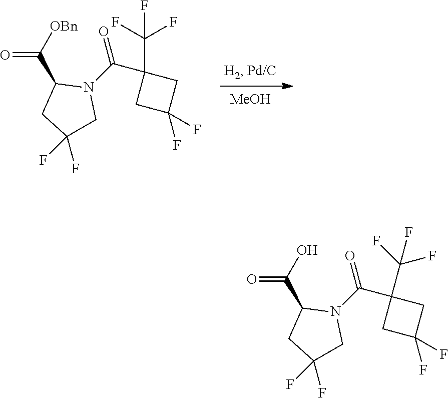 [SMILES: "MeOH" ; O=C(OCc1ccccc1)[C@@H]1CC(F)(F)CN1C(=O)C1(C(F)(F)F)CC(F)(F)C1 ; "H2, Pd/C" ; O=C(O)[C@@H]1CC(F)(F)CN1C(=O)C1(C(F)(F)F)CC(F)(F)C1]

To a solution of benzyl(S)-1-(3,3-difluoro-1-(trifluoromethyl)cyclobutane-1-carbonyl)-4,4-difluoropyrrolidine-2-carboxylate (2.30 g, 5.38 mmol, 1.0 Equiv.) in MeOH (30 mL) was added 10% Pd/C (572 mg, 538 μmol, 0.1 Equiv.) under Ar atmosphere. The suspension was degassed and purged with H₂. The mixture was stirred under H₂ (15 Psi) at 20° C. for 2 hrs. The reaction is filtered with diatomaceous earth and the filter cake was rinsed with MeOH (10 mL×3). Then the combined filtrates were concentrated under reduced pressure. The resulting residue was purified by reversed-phase flash chromatography (C18, MeCN in Water (0.2% formic acid), 35%-65% gradient over 10 min) to give Int. 162 (1.08 g, 3.03 mmol, 56.2% yield, 94.5% purity) as a white solid after lyophilization. LCMS: m/z 336.0 (M−H⁺). ¹H NMR (400 MHz, DMSO-d₆): δ13.03 (s, 1H), 4.68-4.61 (m, 1H), 3.99 (t, J=11.6 Hz, 2H), 3.33-3.23 (m, 4H), 2.94-2.80 (m, 1H), 2.50-2.40 (m, 1H). 17 (s, 12H), 1.12 (s, 6H).

### Int. 172: Preparation of 1-methyl-5′-(4,4,5,5-tetramethyl-1,3,2-dioxaborolan-2-yl)-3′H-spiro[azetidine-3,1′-isobenzofuran]

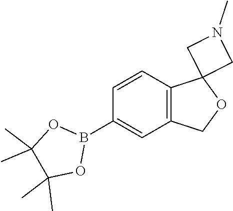 [SMILES: CN1CC2(C1)OCc1cc(B3OC(C)(C)C(C)(C)O3)ccc12]

### Step 1: 5′-bromo-3′H-spiro[azetidine-3,1′-isobenzofuran]

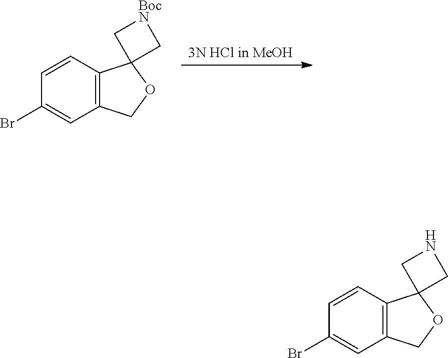 [SMILES: CC(C)(C)OC(=O)N1CC2(C1)OCc1cc(Br)ccc12 ; "3N HCl in MeOH" ; Brc1ccc2c(c1)COC21CNC1]

Tert-butyl 5′-bromo-3′h-spiro[azetidine-3,1′-isobenzofuran]-1-carboxylate (300 mg, 1 Equiv., 882 μmol) was dissolved in 3 mL 3N HCl in MeOH and stirred at 50° C. until the deprotection was complete (monitored via LCMS). The volatile solvent was completely removed to afford the crude deprotection product as the HCl salt. The material can be used without any further purification in the next reaction. LCMS (ESI+): m/z 240.37 (M+H).

### Step 2: 5′-bromo-1-methyl-3′H-spiro[azetidine-3,1′-isobenzofuran]

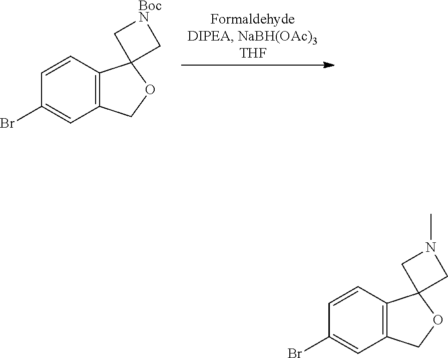 [SMILES: CC(C)(C)OC(=O)N1CC2(C1)OCc1cc(Br)ccc12 ; "FormaldehydeDIPEA, NaBH(OAc)3THF" ; CN1CC2(C1)OCc1cc(Br)ccc12]

Crude 5′-bromo-3′H-spiro[azetidine-3,1′-isobenzofuran]hydrochloride, formaldehyde (98.2 mg, 90.1 μL, 37% Wt, 1.5 Equiv., 1.21 mmol) and DIPEA (313 mg, 421 μL, 3 Equiv., 2.42 mmol) were dissolved in THE (8.67 mL) stirred at 25° C. for 15 mins. Then, NaBH(OAc)₃ (305 mg, 2 Equiv., 1.61 mmol) was added portion-wise to the reaction mixture. The reaction was stirred at room temperature until the starting material was completely consumed (monitored by LCMS). The reaction mixture was diluted with water (20 mL) and extracted with EtOAc (2×20 mL). The combined organic layer was washed with brine, dried over MgSO₄, filtered, and concentrated. The crude mixture was purified by silica gel column chromatography, eluted with DCM/MeOH (95:5). The solvent was removed to afford 5′-bromo-1-methyl-3′H-spiro[azetidine-3,1′-isobenzofuran] (185 mg, 728 mol, 90.3%) as white solid. LCMS (ESI+): m/z 253.96 (M+H).

### Step 3: Int. 172

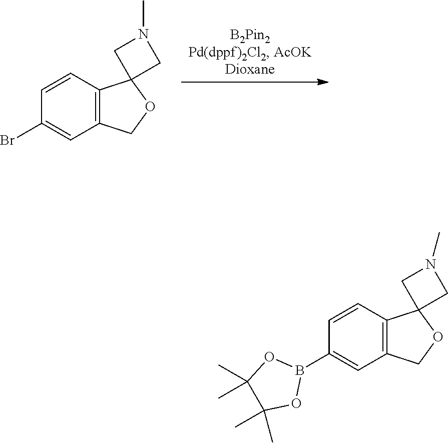 [SMILES: CN1CC2(C1)OCc1cc(Br)ccc12 ; "B2Pin2Pd(dppf)2Cl2, AcOKDioxane" ; CN1CC2(C1)OCc1cc(B3OC(C)(C)C(C)(C)O3)ccc12]

In a sealed tube were placed 5′-bromo-1-methyl-3′H-spiro[azetidine-3,1′-isobenzofuran] (185 mg, 1 Equiv., 544 μmol), Potassium acetate (213 mg, 136 μL, 4 Equiv., 2.18 mmol) and Bis(pinacolato)diborane (414 mg, 3 Equiv., 1.63 mmol) in Dioxane (2.72 mL). The reaction was degassed for 15 min with Ar and then [1,1′-Bis(diphenylphosphino)ferrocene]dichloropalladium(II) Dichloro (22.2 mg, 0.05 Equiv., 27.2 mol) was added. The reaction was sealed and heated to 80° C. until the complete consumption of starting material (monitored by LCMS). Then the reaction was cooled to room temperature, quenched with water, and the organic layers were extracted with EtOAc (3×10 mL). The combined organic layers were washed with water (2×10 mL) and brine (10 mL), dried (Na₂SO₄), concentrated, and purified by flash column chromatography, (DCM:MeOH, 95:5) and concentrated. This resulted in Int. 172 (149 mg, 495 μmol, 68.0%) as brown solid. LCMS (ESI+): m/z 387.52 (M+H).

### Int. 174: (2S,4R)-1-(1-(difluoromethyl)-3,3-difluorocyclobutane-1-carbonyl)-4-fluoropyrrolidine-2-carboxylic acid

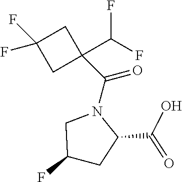 [SMILES: O=C(O)[C@@H]1C[C@@H](F)CN1C(=O)C1(C(F)F)CC(F)(F)C1]

### Step 1: Isopropyl 1-formyl-3,3-dimethoxycyclobutane-1-carboxylate

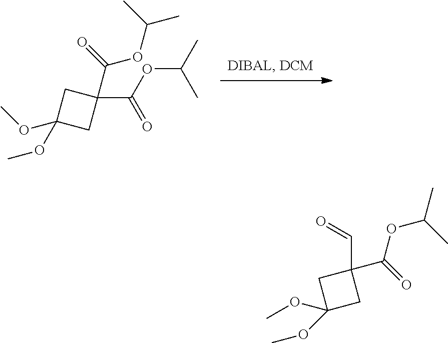 [SMILES: COC1(OC)CC(C(=O)OC(C)C)(C(=O)OC(C)C)C1 ; "DIBAL, DCM" ; COC1(OC)CC(C=O)(C(=O)OC(C)C)C1]

To a stirred solution of 1,1-diisopropyl 3,3-dimethoxycyclobutane-1,1-dicarboxylate (10 g, 34.681 mmol, 1 Equiv.) in DCM (100 mL) was added DIBAL (69.36 mL, 69.362 mmol, 2 Equiv., 1M in DCM) dropwise at −78° C. under argon atmosphere. The resulting mixture was stirred for 4 hr at −78° C. under argon atmosphere. The desired product could be detected by GCMS. The reaction was quenched with 2 N HCl (aq.) at 0° C. The aqueous layer was extracted with CH₂Cl₂ (50 mL×2). The combined organics were dried over anhydrous Na₂SO₄ and concentrated under reduced pressure. The residue was purified by silica gel column chromatography, eluted with Pet. Ether/EtOAc (5:1) to afford isopropyl 1-formyl-3,3-dimethoxycyclobutane-1-carboxylate (2.1 g, 25.0%) as a colorless oil. GCMS: (EIC): m/z 230.00 (M).

### Step 2: Isopropyl 1-formyl-3-oxocyclobutane-1-carboxylate

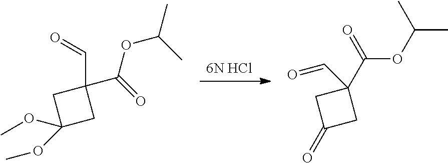

A mixture of isopropyl 1-formyl-3,3-dimethoxycyclobutane-1-carboxylate (2.1 g, 9.120 mmol, 1 Equiv.) in 6N HCl (25 mL) was stirred overnight at room temperature. Desired product could be detected by GCMS. The aqueous layer was extracted with CH₂Cl₂ (50 mL). The organic layer was washed with brine, dried over anhydrous Na₂SO₄ and concentrated under reduced pressure. This resulted in isopropyl 1-formyl-3-oxocyclobutane-1-carboxylate (1.0 g, 53.6%) as a colorless oil which was used in the next reaction without further purification. GCMS (EIC): m/z 184.00 (M).

### Step 3: Isopropyl 1-(difluoromethyl)-3,3-difluorocyclobutane-1-carboxylate

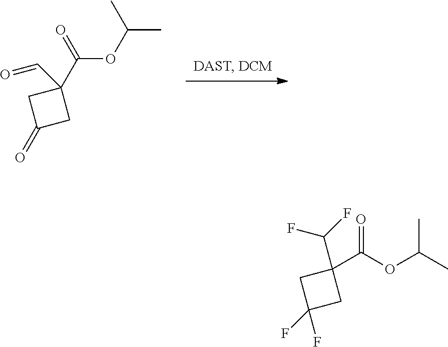

To a stirred solution of isopropyl 1-formyl-3-oxocyclobutane-1-carboxylate (1 g, 5.429 mmol, 1 Equiv.) in DCM (20 mL) was added DAST (4.81 g, 29.860 mmol, 5.5 Equiv.) dropwise at 0° C. under atmosphere of argon. The resulting mixture was stirred overnight at room temperature. Desired product could be detected by GCMS. The reaction was quenched by the addition of sat. NaHCO₃ (aq.) (100 mL) at 0° C. The organics were dried over anhydrous Na₂SO₄ and concentrated under reduced pressure. The residue was purified by silica gel column chromatography, eluted with CH₂Cl₂ to afford isopropyl 1-(difluoromethyl)-3,3-difluorocyclobutane-1-carboxylate (1 g, 72.65%) as a colorless oil. GCMS (EIC): m/z 228.00 (M).

### Step 4: 1-(difluoromethyl)-3,3-difluorocyclobutane-1-carboxylic acid

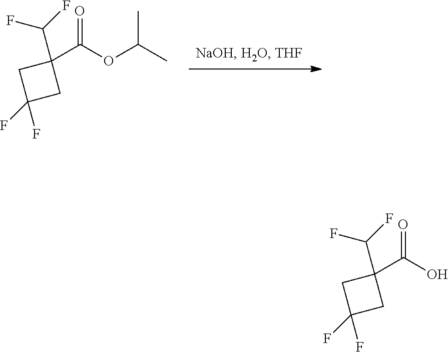

To a stirred solution of isopropyl 1-(difluoromethyl)-3,3-difluorocyclobutane-1-carboxylate (1.5 g, 6.574 mmol, 1 Equiv.) in THF (20 mL) was added dropwise NaOH (0.79 g, 19.722 mmol, 3 Equiv.) in H₂O (20 mL) at 0° C. The resulting mixture was stirred overnight at room temperature. Desired product could be detected by LCMS. The resulting mixture was diluted with water (20 mL) and acidified with HCl (aq.) to pH ˜4. The aqueous layer was extracted with EtOAc (2×30 mL). The combined organics were dried over anhydrous Na₂SO₄ and concentrated under reduced pressure. This resulted in 1-(difluoromethyl)-3,3-difluorocyclobutane-1-carboxylic acid (730 mg, 56.69%) as a colorless oil. The material was used directly in the next step without further purification. LCMS (ESI): m/z 185.00 (M−H⁻).

### Step 5: Methyl (2S,4R)-1-(1-(difluoromethyl)-3,3-difluorocyclobutane-1-carbonyl)-4-fluoropyrrolidine-2-carboxylate

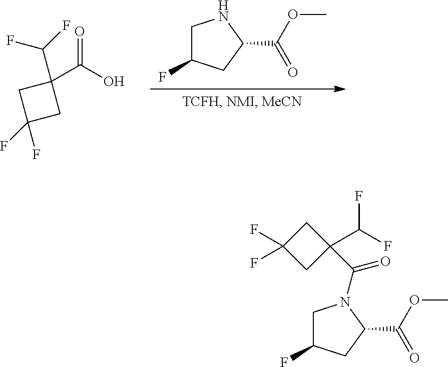

Into a solution of 1-(difluoromethyl)-3,3-difluorocyclobutane-1-carboxylic acid (1 g, 5.373 mmol, 1 Equiv.), TCFH (2.26 g, 8.06 mmol, 1.5 Equiv.) and methyl (2S,4R)-4-fluoropyrrolidine-2-carboxylate (0.87 g, 5.910 mmol, 1.1 Equiv.) in MeCN (20 mL) was added NMI (3.31 g, 40.297 mmol, 7.5 Equiv.) dropwise at 0° C. under nitrogen atmosphere. The mixture was stirred for 16 h at room temperature. The reaction solution was concentrated in vacuo. The residue was purified by reverse \chromatography (20 g C18; MeCN in Water +0.1% formic acid; 10% to 50% gradient in 40 min). This resulted in methyl (2S,4R)-1-[1-(difluoromethyl)-3,3-difluorocyclobutanecarbonyl]-4-fluoropyrrolidine-2-carboxylate (300 mg, 15.94%) as a brown solid upon removal of solvents. LCMS (ESI): m/z 315.24 (M+H⁺). ¹H NMR (400 MHz, DMSO-d₆): δ 2.11 (dddd, J=4.0, 8.9, 14.7, 39.8 Hz, 1H), 2.51-2.67 (m, 2H), 2.85-3.24 (m, 4H), 3.31-3.46 (m, 1H), 3.66-3.99 (m, 3H), 4.55 (t, J=8.7, 8.7 Hz, 1H), 5.41 (dt, J=3.3, 3.3, 52.6 Hz, 1H), 6.47 (t, J=55.2, 55.2 Hz, 1H).

### Step 6: Int 174

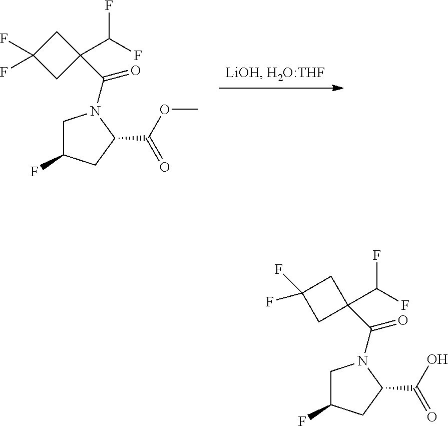

Into a solution of methyl (2S,4R)-1-[1-(difluoromethyl)-3,3-difluorocyclobutanecarbonyl]-4-fluoropyrrolidine-2-carboxylate (600 mg, 1.903 mmol, 1 Equiv.) in THF (10 mL) was added LiOH (136.75 mg, 5.709 mmol, 3 Equiv.) in H₂O (10 mL) at 0° C. The resulting solution was stirred for 16 hr at room temperature. The reaction mixture was concentrated in vacuo to remove THF. The aqueous layer was acidified with 1 N HCl to pH ˜4 and extracted with EtOAc (3×20 mL). The combined organic layers were washed with brine, dried over Na₂SO₄ and concentrated in vacuo. This resulted in Int. 174 (0.496 g, 84.8%) as a white solid. LCMS (ESI): m/z 302.21 (M+H⁺). ¹H NMR (400 MHz, CD₃OD): δ2.07-2.27 (m, 1H), 2.59-2.75 (m, 1H), 2.91-3.16 (m, 3H), 3.20-3.31 (m, 1H), 3.73 (ddd, J=2.7, 12.6, 36.6 Hz, 1H), 3.95 (ddd, J=2.3, 12.6, 17.7 Hz, 1H), 4.63 (t, J=8.8, 8.8 Hz, 1H), 5.35 (dd, J=3.4, 52.1 Hz, 1H), 6.24 (t, J=55.6, 55.6 Hz, 1H).

### Int. 176: 2-((1S,6R)-3-oxabicyclo[4.1.0]heptan-6-yl)-5-(4,4,5,5-tetraethyl-1,3,2-dioxaborolan-2-yl)pyridine

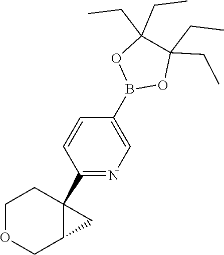 [SMILES: CCC1(CC)OB(c2ccc([C@]34CCOC[C@H]3C4)nc2)OC1(CC)CC]

### Step 1: 5-bromo-2-(3,6-dihydro-2H-pyran-4-yl)pyridine

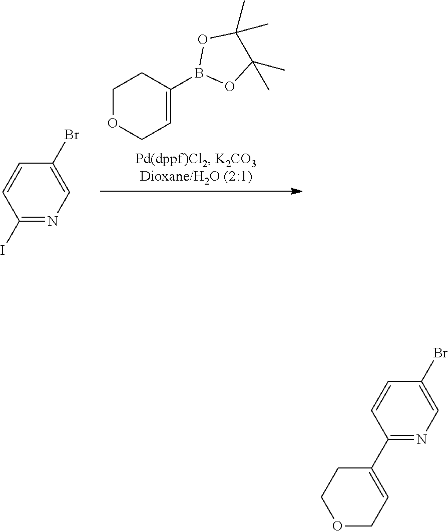

To a stirred solution of 5-bromo-2-iodopyridine (5 g, 17.612 mmol, 1 Equiv.) and 2-(3,6-dihydro-2H-pyran-4-yl)-4,4,5,5-tetramethyl-1,3,2-dioxaborolane (4.44 g, 21.134 mmol, 1.2 Equiv.) in dioxane (100 mL) and H₂O (50 mL) were added PdCl₂(Dppf)·CH₂Cl₂ (1.43 g, 1.761 mmol, 0.1 Equiv.) and K₂CO₃ (4.87 g, 35.224 mmol, 2 Equiv.) at room temperature under nitrogen atmosphere. The resulting mixture was stirred for 6 hr at 80° C. under nitrogen atmosphere. The resulting mixture was diluted with water (50 mL). The aqueous layer was extracted with EtOAc (3×100 mL). The combined organics were dried over anhydrous Na₂SO₄ and concentrated under vacuum. The residue was purified by flash chromatography (PE/EtOAc 3:1) to afford 5-bromo-2-(3,6-dihydro-2H-pyran-4-yl)pyridine (2.8 g, 66.2%) as a light yellow solid. LCMS: (ESI): m/z 240.10 (M+H⁺). ¹H NMR (400 MHz, DMSO-d₆): δ 2.61 (dddq, J=5.6, 3.9, 2.8, 1.4 Hz, 2H), 3.94 (t, J=5.5 Hz, 2H), 4.36 (q, J=2.8 Hz, 2H), 6.70 (tt, J=3.1, 1.6 Hz, 1H), 7.28 (d, J=8.4 Hz, 1H), 7.78 (dd, J=8.5, 2.4 Hz, 1H), 8.61 (d, J=2.4 Hz, 1H).

### Step 2: 2-(3-oxabicyclo[4.1.0]heptan-6-yl)-5-bromopyridine

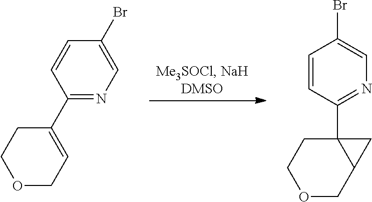

To a stirred mixture of trimethylsulfoxonium chloride Th(3.00 g, 23.324 mmol, 2.00 Equiv.) and NaH (0.93 g, 23.324 mmol, 2.00 Equiv., 60%) in DMSO (30 mL) was added 5-bromo-2-(3,6-dihydro-2H-pyran-4-yl)pyridine (2.8 g, 11.662 mmol, 1 Equiv.) at room temperature under nitrogen atmosphere. The resulting mixture was stirred for overnight at 65° C. under nitrogen atmosphere. The reaction was quenched with sat. NH₄Cl (aq.) at 0° C. and extracted with EtOAc (3×100 mL). The combined organics were dried over anhydrous Na₂SO₄ and concentrated in vacuo. The residue was purified by reversed-phase flash chromatography (C18 silica gel; mobile phase, MeCN in Water with 10 mmol/L NH₄HCO₃, 5% to 95) This resulted in 2-(3-oxabicyclo[4.1.0]heptan-6-yl)-5-bromopyridine (1.1 g, 37.12%) as a light yellow oil. LCMS (ESI): m/z 254.0 (M+H⁺). ¹H NMR (400 MHz, Chloroform-d): δ 1.08 (dd, J=6.3, 4.2 Hz, 1H), 1.27 (dd, J=9.1, 4.2 Hz, 1H), 1.69 (dddd, J=9.1, 6.1, 4.3, 1.5 Hz, 1H), 2.11 (ddd, J=14.2, 8.9, 5.6 Hz, 1H), 2.44 (dt, J=13.9, 4.9 Hz, 1H), 3.46 (ddd, J=11.6, 8.9, 5.1 Hz, 1H), 3.64 (dt, J=11.6, 5.2 Hz, 1H), 3.91-4.09 (m, 2H), 7.12 (d, J=8.3 Hz, 1H), 7.71 (dd, J=8.5, 2.4 Hz, 1H), 8.53 (d, J=2.4 Hz, 1H).

### Step 3: 2-((1S,6R)-3-oxabicyclo[4.1.0]heptan-6-yl)-5-bromopyridine

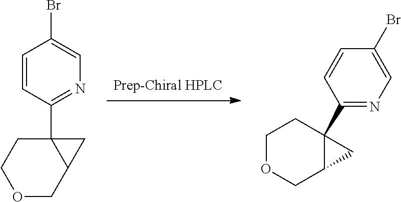 [SMILES: Brc1ccc(C23CCOCC2C3)nc1 ; "Prep-Chiral HPLC" ; Brc1ccc([C@]23CCOC[C@H]2C3)nc1]

The 2-(3-oxabicyclo[4.1.0]heptan-6-yl)-5-bromopyridine (800 mg, 3.148 mmol, 1 Equiv.) was purified by Prep-Chiral-HPLC with the following conditions (Column: CHIRALPAK IG, 2×25 cm, 5 m; Mobile Phase A: Hex(0.5% 2M NH₃-MeOH), Mobile Phase B: MeOH:DCM=1: 1—HPLC; Flow rate: 20 mL/min; Gradient: 10% B) to afford 2-((1S,6R)-3-oxabicyclo[4.1.0]heptan-6-yl)-5-bromopyridine (250 mg, 31.25%) as a colorless oil. LCMS (ESI): m/z 254.00 (M+H⁺).

### Step 4: Int. 176

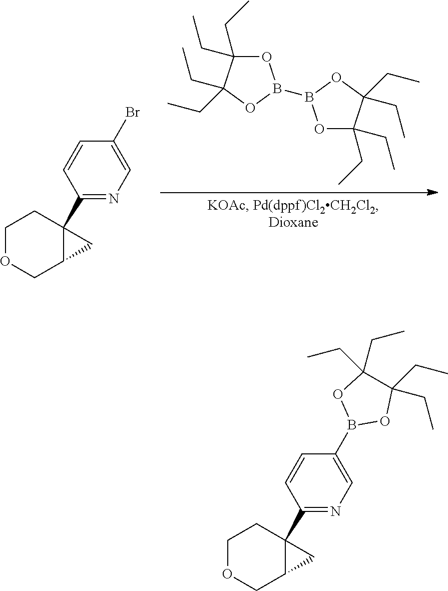

To a stirred solution of 2-((1S,6R)-3-oxabicyclo[4.1.0]heptan-6-yl)-5-bromopyridine (240 mg, 0.944 mmol, 1 Equiv.), KOAc (278.06 mg, 2.83 mmol, 3 Equiv.) and 4,4,5,5-tetraethyl-2-(4,4,5,5-tetraethyl-1,3,2-dioxaborolan-2-yl)-1,3,2-dioxaborolane (414.97 mg, 1.133 mmol, 1.2 Equiv.) in dioxane (10 mL) was added Pd(dppf)Cl₂·CH₂Cl₂ (76.93 mg, 0.094 mmol, 0.1 Equiv.) at room temperature under nitrogen atmosphere. The resulting mixture was stirred for 2 h at 80° C. under nitrogen atmosphere. The resulting mixture was concentrated under vacuum. The residue was purified by Prep-TLC (Pet. Ether/EtOAc 7:1) to afford Int. 176 as a colorless oil. LCMS (ESI): m/z 358.15 (M+H⁺). ¹H NMR (400 MHz, DMSO-d₆): δ 0.91 (t, J=7.4 Hz, 12H), 1.03 (dd, J=6.3, 3.9 Hz, 1H), 1.27 (dd, J=9.0, 3.8 Hz, 1H), 1.58 (q, J=7.5 Hz, 2H), 1.63-1.78 (m, 9H), 1.98 (ddd, J=13.8, 8.6, 5.4 Hz, 1H), 2.53-2.62 (m, 1H), 3.35-3.44 (m, 1H), 3.53 (dt, J=11.1, 5.3 Hz, 1H), 3.81 (d, J=11.3 Hz, 1H), 3.92 (dd, J=11.3, 4.6 Hz, 1H), 7.35 (d, J=8.0 Hz, 1H), 7.92 (dd, J=8.1, 1.8 Hz, 1H), 8.66 (s, 1H).

### Int. 178: Preparation of 8-methyl-3-(4-(4,4,5,5-tetramethyl-1,3,2-dioxaborolan-2-yl)phenyl)-3,8-diazabicyclo[3.2.1]octane

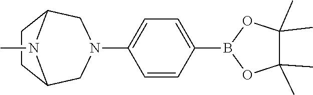

### Step 1: tert-butyl 3-phenyl-3,8-diazabicyclo[3.2.1]octane-8-carboxylate

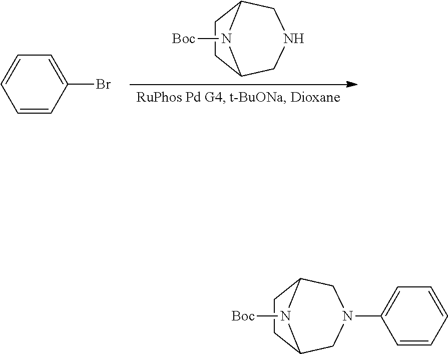

To a solution of bromobenzene (5 g, 31.85 mmol, 3.35 mL, 1 Equiv.) in dioxane (25 mL) was added t-BuONa in dioxane (2 M, 31.85 mL, 2), tert-butyl 3,8-diazabicyclo[3.2.1]octane-8-carboxylate (7.44 g, 35.03 mmol, 1.1 Equiv.) and RuPhos Pd G4 (2.71 g, 3.18 mmol, 0.1 Equiv.) under N₂. The mixture was stirred at 90° C. for 12 hr under N₂. LCMS showed starting material was consumed desired mass was detected. The reaction mixture was cooled to room temperature and diluted with water (50 mL), extracted with EtOAc (2×50 mL). The combined organics were washed with brine (50 mL), dried over Na₂SO₄, filtered, and concentrated under reduced. The resulting residue was purified by flash silica gel chromatography (Pet. Ether/EtOAc, 5:1) to afford tert-butyl 3-phenyl-3,8-diazabicyclo[3.2.1]octane-8-carboxylate (9 g, 31.2 mmol, 97%) as a white solid. LCMS (ESI+): m/z 289.3 (M+H⁺). ¹H NMR (400 MHz, CDCl₃): δ 7.32-7.28 (m, 2H), 6.90-6.81 (m, 3H), 4.49-4.30 (m, 2H), 3.44 (dd, J=1.9, 11.1 Hz, 2H), 3.02 (br s, 2H), 2.02-1.85 (m, 4H), 1.51 (s, 9H).

### Step 2: tert-butyl 3-(4-bromophenyl)-3,8-diazabicyclo[3.2.1]octane-8-carboxylate

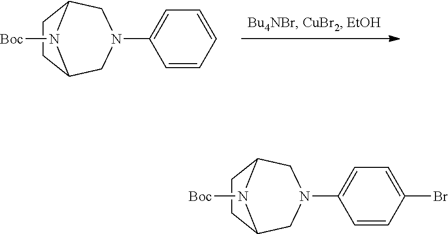

To a solution of tert-butyl 3-phenyl-3,8-diazabicyclo[3.2.1]octane-8-carboxylate (9 g, 31.21 mmol, 1 Equiv.) ethanol (540 mL) was added tetrabutylammonium; bromide (10.06 g, 31.21 mmol, 1 Equiv.) and dibromocopper (10.46 g, 46.81 mmol, 2.19 mL, 1.5 Equiv.) under N₂. The mixture was stirred at 25° C. for 12 hrs. LCMS showed starting material was consumed and desired mass was detected. The reaction mixture was filtered, and the filter cake was rinsed with EtOAc (3×20 mL). Then the combined filtrates were concentrated under reduced pressure. The resulting residue was purified by flash silica gel chromatography (Pet. Ether/EtOAc, 5:1) to afford tert-butyl 3-(4-bromophenyl)-3,8-diazabicyclo[3.2.1]octane-8-carboxylate (5.0 g, 13.6 mmol, 44%) as a white solid. LCMS (ESI+): m/z 367.1 (M+H⁺). ¹H NMR (400 MHz, CDCl₃): δ 7.33 (d, J=9.0 Hz, 2H), 6.70 (br d, J=9.0 Hz, 2H), 4.36 (br s, 2H), 3.40-3.30 (m, 2H), 2.97 (br s, 2H), 2.02-1.79 (m, 4H), 1.48 (s, 9H).

### Step 3: tert-butyl 3-(4-(4,4,5,5-tetramethyl-1,3,2-dioxaborolan-2-yl)phenyl)-3,8-diazabicyclo[3.2.1]octane-8-carboxylate

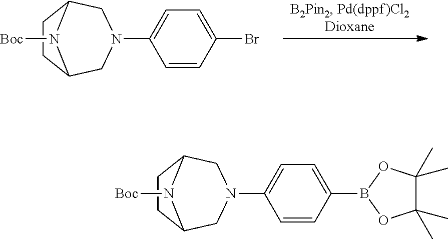 [SMILES: CC(C)(C)OC(=O)N1C2CCC1CN(c1ccc(Br)cc1)C2 ; "B2Pin2, Pd(dppf)Cl2Dioxane" ; CC(C)(C)OC(=O)N1C2CCC1CN(c1ccc(B3OC(C)(C)C(C)(C)O3)cc1)C2]

To a solution of tert-butyl 3-(4-bromophenyl)-3,8-diazabicyclo[3.2.1]octane-8-carboxylate (4 g, 10.89 mmol, 1 Equiv.) dioxane (40 mL) was added bis(pinacolato)diboron (4.15 g, 16.34 mmol, 1.5 Equiv.), potassium acetate (3.21 g, 32.67 mmol, 3 Equiv.) and Pd(dppf)Cl₂ (796.89 mg, 1.09 mmol, 0.1 Equiv.) under N₂. The mixture was stirred at 80° C. for 12 hrs under N₂. LCMS showed starting material was consumed and desired mass was detected. The reaction mixture was filtered, and the filter cake was rinsed with EtOAc (45 mL). Then the combined filtrates were concentrated under reduced pressure. The resulting residue was purified by flash silica gel chromatography, eluted with Pet. Ether/EtOAc (4:1) to afford a yellow solid. The yellow solid was triturated with Pet. Ether (30 mL) at 25° C. for 30 min, filtered. The filter cake was concentrated to give tert-butyl 3-(4-(4,4,5,5-tetramethyl-1,3,2-dioxaborolan-2-yl)phenyl)-3,8-diazabicyclo[3.2.1]octane-8-carboxylate (3.4 g, 8.21 mmol, 75%) as a white solid. LCMS (ESI+): m/z 415.2 (M+H⁺). ¹H NMR (400 MHz, CDCl₃): δ 7.71 (d, J=8.5 Hz, 2H), 6.82 (d, J=8.5 Hz, 2H), 4.48-4.27 (m, 2H), 3.49 (dd, J=1.8, 11.3 Hz, 2H), 3.04 (s, 2H), 2.00-1.80 (m, 4H), 1.48 (s, 9H), 1.38-1.30 (m, 12H).

### Step 4: 3-(4-(4,4,5,5-tetramethyl-1,3,2-dioxaborolan-2-yl)phenyl)-3,8-diazabicyclo[3.2.1]octane

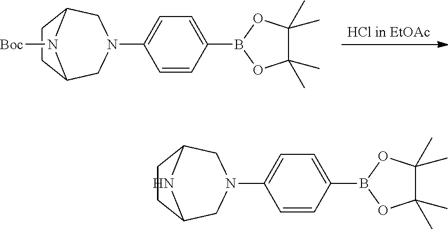 [SMILES: CC(C)(C)OC(=O)N1C2CCC1CN(c1ccc(B3OC(C)(C)C(C)(C)O3)cc1)C2 ; "HCl in EtOAc" ; CC1(C)OB(c2ccc(N3CC4CCC(C3)N4)cc2)OC1(C)C]

To a solution of tert-butyl 3-(4-(4,4,5,5-tetramethyl-1,3,2-dioxaborolan-2-yl)phenyl)-3,8-diazabicyclo[3.2.1]octane-8-carboxylate (4.1 g, 9.90 mmol, 1 Equiv.) in EtOAc (20 mL) was added HCl/EtOAc (4 M, 40 mL, 16.17 Equiv.) dropwise. The mixture was stirred at 25° C. untill LCMS showed the consumption of starting material. The mixture was filtered with EtOAc (30 mL) and the filter cake was concentrated under reduced pressure to give 3-(4-(4,4,5,5-tetramethyl-1,3,2-dioxaborolan-2-yl)phenyl)-3,8-diazabicyclo[3.2.1]octane (2.6 g, 7.4 mmol, 75%, HCl Salt) as a white solid. LCMS (ESI+): m/z 315.2 (M+H⁺). ¹H NMR (400 MHz, MeOD-d6): δ7.65 (d, J=8.8 Hz, 2H), 6.93 (d, J=8.8 Hz, 2H), 4.19 (s, 2H), 3.80 (dd, J=2.4, 13.1 Hz, 2H), 3.13 (d, J=12.1 Hz, 2H), 2.13 (s, 4H), 1.33 (s, 12H)

### Step 5: Int. 178

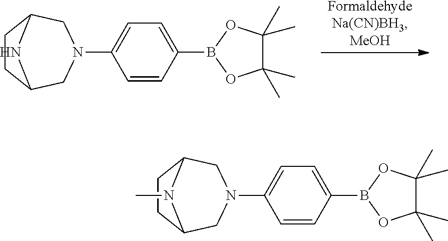

To a solution of 3-(4-(4,4,5,5-tetramethyl-1,3,2-dioxaborolan-2-yl)phenyl)-3,8-diazabicyclo[3.2.1]octane (2.4 g, 6.84 mmol, 1 Equiv., HCl) in methanol (24 mL) was added formaldehyde (2.78 g, 34.22 mmol, 2.55 mL, 37% purity, 5 Equiv.). The mixture was stirred for 30 min under N₂. Then Na(CN)BH₃ (860.14 mg, 13.69 mmol, 2 Equiv.) was added to the reaction. The mixture was stirred at 25° C. until the consumption of starting material was observed (LCMS). The reaction mixture was diluted by water (50 mL), extracted with EtOAc (2×50 mL). The combined organics were washed with brine (50 mL), dried over Na₂SO₄, filtered, and concentrated under reduced pressure. The resulting residue was triturated with methyl tert-butyl ether (20 mL), then filtered. The filter cake was collected to give Int. 178 (1.07 g, 3.26 mmol, 48%) as a white solid. LCMS (ESI+): m/z 329.2 (M+H⁺). ¹H NMR (400 MHz, MeOD-d6): δ7.58 (d, J=8.6 Hz, 2H), 6.80 (d, J=8.6 Hz, 2H), 3.53-3.48 (m, 2H), 3.35-3.32 (m, 2H), 2.99 (br d, J=10.0 Hz, 2H), 2.36 (s, 3H), 2.14-2.06 (m, 2H), 1.81-1.74 (m, 2H), 1.32 (s, 12H)

### Int. 179: Preparation of (1S,4S)-2-methyl-5-(4-(4,4,5,5-tetramethyl-1,3,2-dioxaborolan-2-yl)phenyl)-2,5-diazabicyclo[2.2.1]heptane

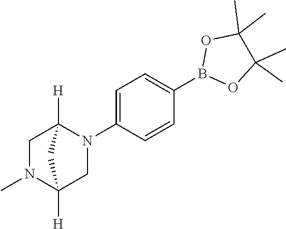

### Step 1: (1S,4S)-2-(4-bromophenyl)-5-methyl-2,5-diazabicyclo[2.2.1]heptane

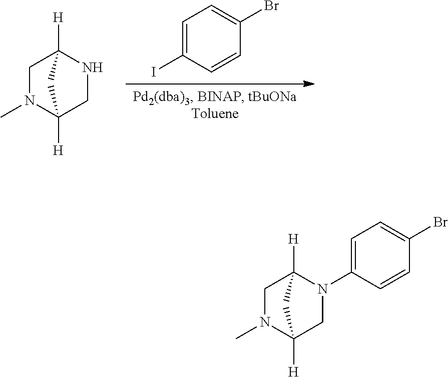

A mixture of (1S,4S)-2-methyl-2,5-diazabicyclo[2.2.1]heptane; dihydrobromide (3.90 g, 14.2 mmol, 1.0 Equiv.), 1-bromo-4-iodo-benzene (4.03 g, 14.2 mmol, 1.0 Equiv.), Pd₂(dba)₃ (391 mg, 427 μmol, 0.03 Equiv.), t-BuONa (3.42 g, 35.6 mmol, 2.5 Equiv.) and [1-(2-diphenylphosphanyl-1-naphthyl)-2-naphthyl]-diphenyl-phosphane (88.63 mg, 142.34 μmol, 0.01 Equiv.) in toluene (160 mL) was degassed and purged with N₂, then the mixture was stirred at 110° C. for 12 hr under N₂ atmosphere. After cooling to room temperature, the reaction mixture was concentrated to remove solvent. Water (50 mL) was added and the aqueous phase was extracted with EtOAc (30 mL×3), the combined organic phase was washed with brine (30 mL×3), dried over Na₂SO₄, filtered, and concentrated. The resulting residue was dissolved in water (50 mL) and acidified to pH of 5-6 with HCl (1 M), the aqueous phase was extracted with MTBE (30 mL×3). Then the aqueous phase was alkalized to pH of 7-8 with saturated NaHCO₃ and extracted with ethyl acetate (50 mL×3), the combined organic layers were dried over Na₂SO₄, filtered and the filtrate was concentrated to give (1S,4S)-2-(4-bromophenyl)-5-methyl-2,5-diazabicyclo[2.2.1]heptane (1.70 g, 6.12 mmol, 43.01% yield) as a yellow oil. LCMS (ESI+): m/z 267.2 (M+H⁺).

### Step 2: Int. 179

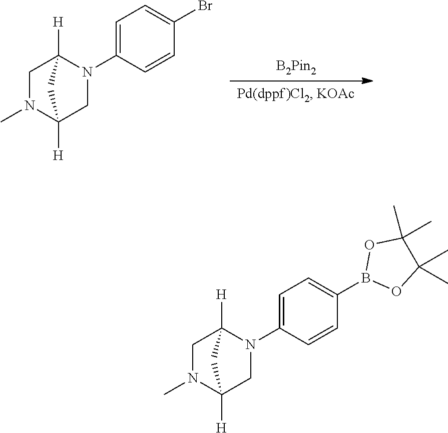

A mixture of (1S,4S)-2-(4-bromophenyl)-5-methyl-2,5-diazabicyclo[2.2.1]heptane (1.70 g, 6.36 mmol, 1.0 Equiv.), 4,4,5,5-tetramethyl-2-(4,4,5,5-tetramethyl-1,3,2-dioxaborolan-2-yl)-1,3,2-dioxaborolane (2.75 g, 10.8 mmol, 1.7 Equiv.), KOAc (1.56 g, 15.9 mmol, 2.5 Equiv.), Pd(dppf)Cl₂ (232 mg, 318 μmol, 0.05 Equiv.) in dioxane (20 mL) was degassed and purged with N₂. The mixture was stirred at 100° C. for 12 hr under N₂ atmosphere. After cooling to room temperature, the reaction mixture was concentrated. The resulting residue was diluted with water (30 mL) and EtOAc (20 mL), the resultant mixture was filtered, and the filter cake was rinsed with EtOAc (20 mL×3), the resulting biphasic solution was separated, then the aqueous phase was extracted with EtOAc (30 mL×3), the combined organic layers were dried over Na₂SO₄, filtered and the filtrate was concentrated. The resulting residue was purified by silica gel column chromatography (Pet. Ether/EtOAc, 1:0 to 20:1) to afford Int. 179 (650 mg, 2.02 mmol, 31.74% yield) as a brown solid. LCMS (ESI+): m/z 315.2 (M+H⁺). ¹H NMR (400 MHz, DMSO-d6): δ7.44 (d, J=8.0 Hz, 2H), 6.53 (d, J=8.0 Hz, 2H), 4.32 (s, 1H), 3.30 (d, J=9.2 Hz, 2H), 3.17 (d, J=9.2 Hz, 1H), 2.78 (d, J=8.8 Hz, 1H), 2.42 (d, J=9.2 Hz, 1H), 2.24 (s, 3H), 1.84-1.91 (m, 1H), 1.75 (d, J=8.8 Hz, 1H), 1.25 (s, 12H).

### Int. 180: (2S,4R)-1-(3,3-difluoro-1-methylcyclobutane-1-carbonyl)-4-fluoropyrrolidine-2-carboxylic acid

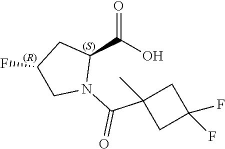

### Step 1: methyl (2S,4R)-4-fluoropyrrolidine-2-carboxylate hydrochloride

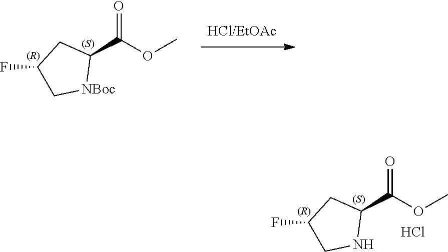

To a solution of 1-(tert-butyl) 2-methyl (2S,4R)-4-fluoropyrrolidine-1,2-dicarboxylate (5 g, 20.22 mmol, 1 Equiv.) in EtOAc (10 mL) was added dropwise HCl/EtOAc (4 M, 200 mL, 39.56 Equiv.). The mixture was stirred until the complete consumption of starting material (LCMS). The reaction was concentrated under reduced pressure to afford methyl (2S,4R)-4-fluoropyrrolidine-2-carboxylate hydrochloride (3.5 g, 17.16 mmol, 84.84% yield, HCl salt) as a white solid. ¹H NMR (400 MHz, DMSO-d₆): δ 10.25 (br d, J=1.1 Hz, 1H), 5.57-5.32 (m, 1H), 4.55 (dd, J=7.3, 11.3 Hz, 1H), 3.66-3.34 (m, 3H), 2.79-2.51 (m, 3H), 2.43-2.21 (m, 1H).

### Step 2: methyl (2S,4R)-4-fluoropyrrolidine-2-carboxylate hydrochloride

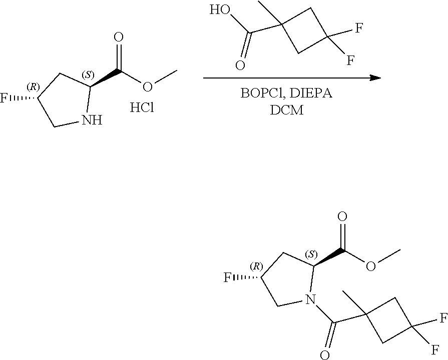

To a solution of 3,3-difluoro-1-methyl-cyclobutanecarboxylic acid (2.70 g, 17.97 mmol, 1.1 Equiv.) in DCM (30 mL) was added DIEPA (2.11 g, 16.34 mmol, 2.85 mL, 1 Equiv.). The mixture was stirred at 20° C. for 10 min. 3-[chloro-(2-oxooxazolidin-3-yl)phosphoryl]oxazolidin-2-one (4.58 g, 17.97 mmol, 1.1 Equiv.) was added to the mixture. Methyl (2S,4R)-4-fluoropyrrolidine-2-carboxylate (3 g, 16.34 mmol, 1 Equiv., HCl) and DIEPA (4.22 g, 32.68 mmol, 5.69 mL, 2 Equiv.) were added to the mixture. The mixture was stirred at room temperature until the complete consumption of staring material (LCMS). The reaction mixture was quenched by water (30 mL), extracted with DCM (2×30 mL). The combined organics were washed with NaHCO₃ (2×20 mL), brine (20 mL), dried over Na₂SO₄, filtered, and concentrated under reduced pressure to afford methyl(2S,4R)-1-(3,3-difluoro-1-methyl-cyclobutanecarbonyl)-4-fluoro-pyrrolidine-2-carboxylate (4.5 g, 14.82 mmol, 90.69%) as a yellow gum. LCMS (ESI+): m/z 280.2 (M+H⁺).

### Step 3: Int. 180

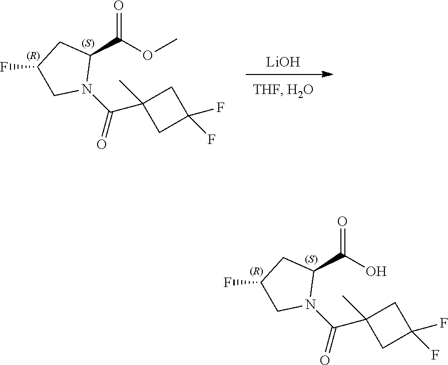

To a solution of methyl (2S,4R)-1-(3,3-difluoro-1-methyl-cyclobutanecarbonyl)-4-fluoro-pyrrolidine-2-carboxylate (4.5 g, 16.11 mmol, 1 Equiv.) in THF (20 mL) was added LiOH·H₂O (2 M, 12.09 mL, 1.5 Equiv.). The reaction was stirred at 20° C. until the complete consumption of starting material was observed (LCMS). The reaction mixture was concentrated under reduced pressure to remove THF, extracted with MTBE (2×20 mL) and acidified by 1N HCl to pH=1˜2 at 0° C. The aqueous phase was extracted with EtOAc (2×20 mL). The combined organics were washed with brine (20 mL), dried over Na₂SO₄, filtered, and concentrated under reduced pressure to afford Int. 180 (3.85 g, 13.58 mmol, 84.28%) as a white solid. LCMS (ESI+): m/z 266.00 (M+H⁺). ¹H NMR (400 MHz, CDCl₃): 5.40-5.20 (m, 1H), 4.79 (t, J=8.6 Hz, 1H), 3.77 (ddd, J=1.9, 12.6, 17.9 Hz, 1H), 3.66-3.47 (m, 1H), 3.23-2.95 (m, 2H), 2.70-2.37 (m, 4H), 1.55 (s, 3H).

### Int. 181: 2,2-dimethyl-4-(4-(4,4,5,5-tetramethyl-1,3,2-dioxaborolan-2-yl)phenyl)morpholine

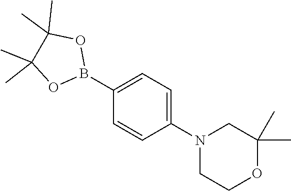

### Step 1: 4-(4-bromophenyl)-2,2-dimethylmorpholine

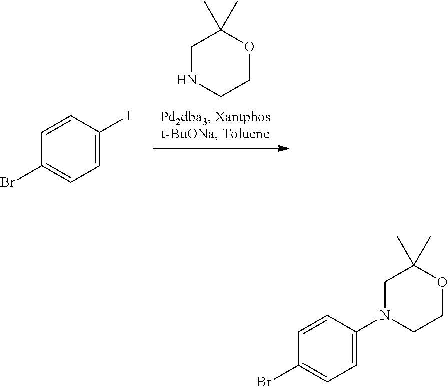

A solution of 4-bromoiodobenzene (2 g, 7.069 mmol, 1 Equiv.), 2,2-dimethylmorpholine (1.63 g, 14.14 mmol, 2 Equiv.), XantPhos (409.06 mg, 0.707 mmol, 0.1 Equiv.), t-BuONa (4.24 mL, 8.483 mmol, 1.20 Equiv.) and Pd₂dba₃ (323.69 mg, 0.353 mmol, 0.05 Equiv.) in Toluene (20 mL) was stirred for 16 hr at 100° C. under N₂ atmosphere. The resulting mixture was diluted with water (50 mL) and then extracted with EtOAc (3×50 mL). The combined organic layers were washed with brine (2×20 mL) and dried over anhydrous Na₂SO₄. After filtration, the filtrate was concentrated under vacuum. The residue was purified by reversed-phase flash chromatography with the following conditions: C18; mobile phase, MeCN in Water (0.1% FA), 0% to 100% gradient in 20 min. This resulted in 4-(4-bromophenyl)-2,2-dimethylmorpholine (1.4 g, 73.3%) as a brown oil. LCMS: (ESI): m/z 270.01 (M+H⁺). ¹H NMR (400 MHz, DMSO-d₆): δ 1.21 (s, 6H), 2.93 (s, 2H), 2.98-3.08 (m, 2H), 3.67-3.77 (m, 2H), 6.84-6.94 (m, 2H), 7.29-7.38 (m, 2H).

### Step 2: Int. 181

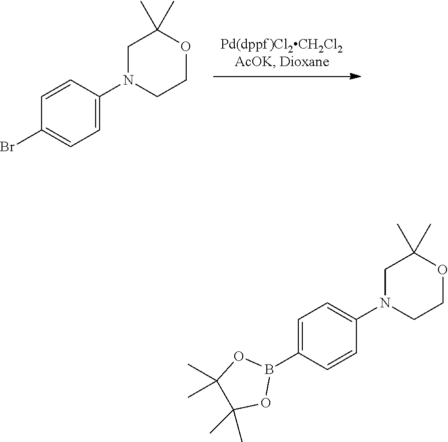 [SMILES: CC1(C)CN(c2ccc(Br)cc2)CCO1 ; "Pd(dppf)Cl2·CH2Cl2AcOK, Dioxane" ; CC1(C)CN(c2ccc(B3OC(C)(C)C(C)(C)O3)cc2)CCO1]

A solution of 4-(4-bromophenyl)-2,2-dimethylmorpholine (1.41 g, 5.219 mmol, 1 Equiv.), bis(pinacolato)diboron (1.46 g, 5.741 mmol, 1.1 Equiv.), AcOK (1.54 g, 15.657 mmol, 3 Equiv.) and Pd(dppf)Cl₂CH₂Cl₂ (425.14 mg, 0.522 mmol, 0.1 Equiv.) in 1,4-dioxane (15 mL) was stirred for 16 h at 80° C. under N₂ atmosphere. The resulting mixture was diluted with EtOAc (100 mL) and then filtered. The filter cake was washed with EtOAc (2×50 mL). The combined filtrates were concentrated under vacuum. The residue was purified FCC (PE/EtOAc 4:1) to afford Int. 181 (1.45 g, 87.3%) as an off-white solid. LCMS (ESI): m/z 318.20 (M+H⁺). ¹H NMR (400 MHz, DMSO-d₆): δ 1.23 (d, J=20.8 Hz, 18H), 3.03 (s, 2H), 3.08-3.16 (m, 2H), 3.68-3.76 (m, 2H), 6.85-6.92 (m, 2H), 7.47-7.53 (m, 2H).

### Int. 182: 4-(2-methoxy-4-(4,4,5,5-tetramethyl-1,3,2-dioxaborolan-2-yl)phenyl)morpholine

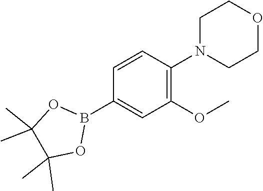 [SMILES: COc1cc(B2OC(C)(C)C(C)(C)O2)ccc1N1CCOCC1]

### Step 1: 4-(4-bromo-2-methoxyphenyl)morpholine

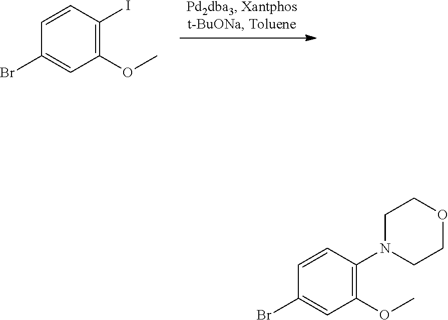

A solution of morpholine (1.11 g, 12.8 mmol, 2 Equiv.), 4-bromo-1-iodo-2-methoxybenzene (2 g, 6.391 mmol, 1 Equiv.), XantPhos (369.81 mg, 0.639 mmol, 0.1 Equiv.), t-BuONa (3.83 mL, 7.669 mmol, 1.2 Equiv.) and Pd₂dba₃ (292.63 mg, 0.32 mmol, 0.05 Equiv.) in toluene (20 mL) was stirred for 16 hr at 100° C. under N₂ atmosphere. The resulting mixture was diluted with water (50 mL) and then extracted with EtOAc (3×50 mL). The combined organic layers were washed with brine (3×50 mL) and dried over anhydrous Na₂SO₄. After filtration, the filtrate was concentrated under vacuum. The residue was purified by reversed-phase flash chromatography with the following conditions: C18; mobile phase, MeCN in Water (0.1% FA), 0% to 100%. This resulted in 4-(4-bromo-2-methoxyphenyl)morpholine (1.1 g, 63.2%) as a light yellow solid upon the removal of solvent. LCMS: (ESI): m/z 271.95 (M+H⁺). ¹H NMR (400 MHz, DMSO-d₆): δ 2.88-2.96 (m, 4H), 3.70 (d, J=9.3 Hz, 4H), 3.80 (s, 3H), 6.82 (d, J=8.4 Hz, 1H), 7.03-7.10 (m, 2H).

### Step 2: Int. 182

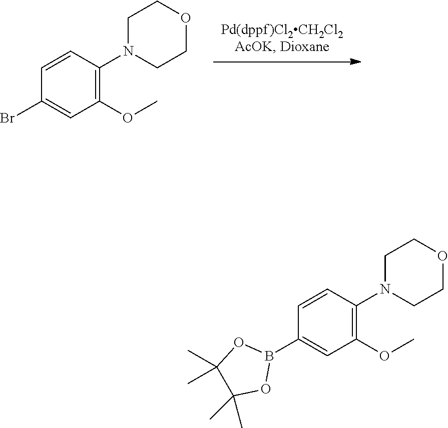 [SMILES: COc1cc(Br)ccc1N1CCOCC1 ; "Pd(dppf)Cl2·CH2Cl2AcOK, Dioxane" ; COc1cc(B2OC(C)(C)C(C)(C)O2)ccc1N1CCOCC1]

A solution of 4-(4-bromo-2-methoxyphenyl)morpholine (1.4 g, 5.1 mmol, 1 Equiv.), bis(pinacolato)diboron (1.44 g, 5.7 mmol, 1.1 Equiv.), AcOK (1.51 g, 15.4 mmol, 3 Equiv.) and Pd(dppf)Cl₂·CH₂Cl₂ (419.07 mg, 0.514 mmol, 0.1 Equiv.) in 1,4-dioxane (20 mL) was stirred for 16 hr at 80° C. under N₂ atmosphere. The resulting mixture was diluted with water (50 mL) and then extracted with EtOAc (3×50 mL). The combined organic layers were washed with brine (50 mL) and dried over anhydrous Na₂SO₄. After filtration, the filtrate was concentrated under vacuum. The residue was purified by FCC (PE/EtOAc 3:1) to afford Int. 182 (1.28 g, 78.1%) as an off-white solid. LCMS (ESI): m/z 320.20 (M+H⁺). ¹H NMR (400 MHz, DMSO-d₆): δ 1.28 (s, 12H), 2.95-3.03 (m, 4H), 3.72 (t, J=4.6 Hz, 4H), 3.79 (s, 3H), 6.87 (d, J=7.8 Hz, 1H), 7.12 (d, J=1.3 Hz, 1H), 7.24 (dd, J=7.8, 1.3 Hz, 1H).

### Int. 183: 1-(pyridin-2-ylmethyl)-4-(4-(4,4,5,5-tetramethyl-1,3,2-dioxaborolan-2-yl)phenyl)piperazine

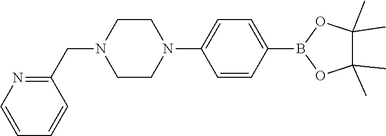 [SMILES: CC1(C)OB(c2ccc(N3CCN(Cc4ccccn4)CC3)cc2)OC1(C)C]

### Step 1: Int. 183

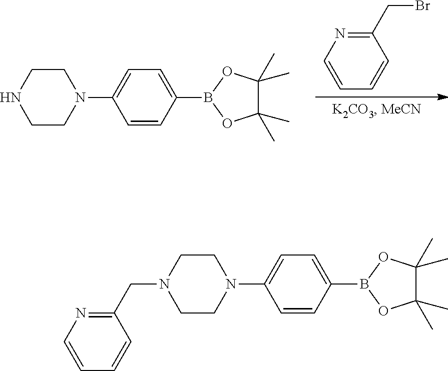

To a solution of 1-[4-(4,4,5,5-tetramethyl-1,3,2-dioxaborolan-2-yl)phenyl]piperazine (8.7 g, 30.19 mmol, 1 Equiv.) in MeCN (200 mL) was added 2-(bromomethyl)pyridine (5.71 g, 33.21 mmol, 1.1 Equiv.) and K₂CO₃ (16.69 g, 120.75 mmol, 4 eq). The mixture was stirred at 60° C. for 12 hrs. LCMS showed starting material was consumed and desired mass was detected. The reaction mixture was concentrated under reduced pressure to afford a crude residue. The residue was triturated with Pet. Ether (20 mL), then filtered, the filter cake was dried and collected to afford Int. 183 (8.3 g, 21.9 mmol, 73%) as a yellow solid. LCMS (ESI+): m/z 380.20 (M+H⁺). ¹H NMR (400 MHz, CDCl₃): δ 8.65-8.55 (m, 1H), 7.78-7.62 (m, 3H), 7.46 (br d, 1H), 7.19 (dd, 1H), 6.89 (d, 2H), 3.74 (s, 2H), 3.31 (br d, 4H), 2.69 (br s, 4H), 1.33 (s, 12H).

### Int. 184: Preparation of (S)-3-(methoxymethyl)-4-(4-(4,4,5,5-tetramethyl-1,3,2-dioxaborolan-2-yl)phenyl)morpholine

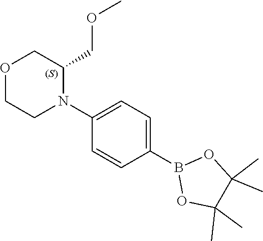

### Step 1: (S)-4-(4-bromophenyl)-3-(methoxymethyl)morpholine

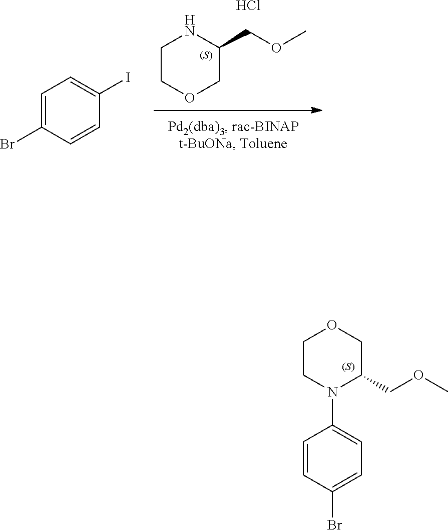

A mixture of 1-bromo-4-iodo-benzene (3.80 g, 13.4 mmol, 1.0 Equiv.), (3S)-3-(methoxymethyl)morpholine (2.03 g, 12.1 mmol, 0.9 Equiv., HCl), Pd₂(dba)₃ (123 mg, 134 mol, 0.01 Equiv.), BINAP (83.6 mg, 134 μmol, 0.01 Equiv.) and t-BuONa (3.87 g, 40.3 mmol, 3.0 Equiv.) in toluene (50 mL) was degassed and purged with N₂. The mixture was stirred at 80° C. for 10 hr under N₂ atmosphere. The toluene was removed in vacuum, then water (50 mL) and saturated NH₄Cl (20 mL) were added, and the aqueous phase was extracted with EtOAc (50 mL×3), the combined organic phase was dried with anhydrous Na₂SO₄, filtered, and concentrated in vacuum. The resulting residue was purified by silica gel column chromatography (Pet. Ether/EtOAc, 1:0 to 10:1) to afford (S)-4-(4-bromophenyl)-3-(methoxymethyl)morpholine (2.80 g, 9.78 mmol, 72.8%) as brown oil. LCMS (ESI+): m/z 286.3/288.3 (M+H⁺).

### Step 2: Int. 184

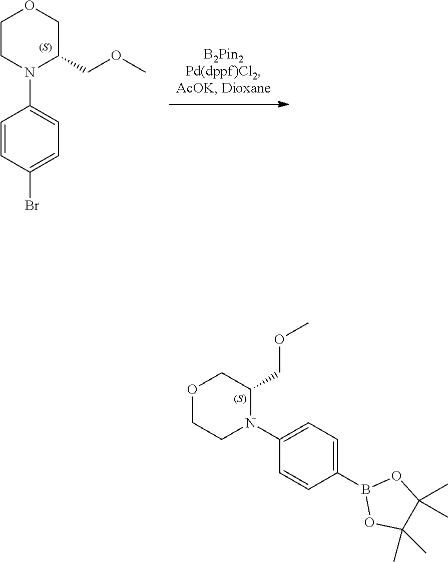

A mixture of (3S)-4-(4-bromophenyl)-3-(methoxymethyl)morpholine (2.60 g, 9.09 mmol, 1.0 Equiv.), Pd(dppf)Cl₂ (332 mg, 454 μmol, 0.05 Equiv.), 4,4,5,5-tetramethyl-2-(4,4,5,5-tetramethyl-1,3,2-dioxaborolan-2-yl)-1,3,2-dioxaborolane (2.54 g, 9.99 mmol, 1.1 Equiv.) and KOAc (2.23 g, 22.7 mmol, 2.5 Equiv.) in dioxane (30 mL) was degassed and purged with N₂. The reaction mixture was stirred at 100° C. for 5 hr under N₂ atmosphere. The dioxane was removed in vacuum, water (30 mL) was added and the aqueous phase was extracted with EtOAc (30 mL×3), the combined organic phase was dried with anhydrous Na₂SO₄, filtered and concentrated in vacuum. The residue was subsequently purified by silica gel column chromatography (Pet. Ether/EtOAc, 1:0 to 10:1) to afford Int. 18 (1.30 g, 3.90 mmol, 42.9%) as white solid. LCMS (ESI+): m/z 334.2 (M+H⁺). ¹H NMR (400 MHz, CDCl₃): δ7.79-7.68 (m, 2H), 6.85 (d, J=8.4 Hz, 2H), 4.15 (d, J=11.6 Hz, 1H), 4.05-3.96 (m, 1H), 3.84 (d, J=10.0 Hz, 1H), 3.77-3.68 (m, 3H), 3.32 (s, 3H), 3.30 (s, 2H), 3.19-3.09 (m, 1H), 1.34 (s, 12H).

### Int. 186: 4-fluoro-1-methyl-4-(4-(4,4,5,5-tetramethyl-1,3,2-dioxaborolan-2-yl)phenyl)piperidine

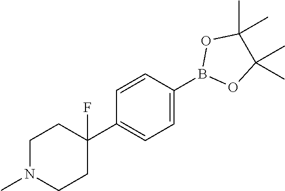

### Step 1: 4-(4-bromophenyl)-1-methylpiperidin-4-ol

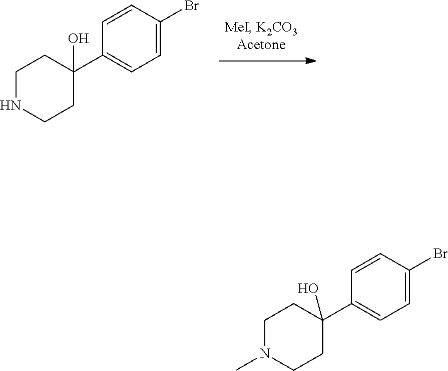

To a solution of 4-(4-bromophenyl)piperidin-4-ol (5 g, 19.520 mmol, 1 Equiv.) in acetone (20 mL) was added MeI (3.05 g, 21.5 mmol, 1.1 Equiv.) and K₂CO₃ (4.05 g, 29.3 mmol, 1.5 Equiv.). The resulting mixture was stirred for 1 h at room temperature under nitrogen atmosphere. The product was detected by LCMS. The reaction was filtered, and the filter cake was washed with EtOAc. The combined organics were dried over anhydrous Na₂SO₄ and concentrated in vacuo. The residue was purified by reversed-phase flash chromatography (MeCN in Water with 0.1% FA 5% to 100%) to afford 4-(4-bromophenyl)-1-methylpiperidin-4-ol (1 g, 18.96%) as a white solid after removal of solvents. LCMS: (ESI): m/z 270.00 (M+H⁺).

### Step 2: 4-(4-bromophenyl)-4-fluoro-1-methylpiperidine

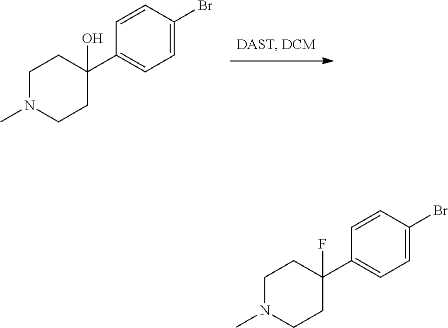

To a solution of 4-(4-bromophenyl)-1-methylpiperidin-4-ol (2 g, 7.403 mmol, 1 Equiv.) in DCM (30 mL) was added dropwise DAST (2.39 g, 14.806 mmol, 2 Equiv.) in DCM (30 mL) at −78° C. under nitrogen atmosphere. The resulting mixture was stirred warmed to RT and stirred overnight. The desired product could be detected by LCMS. The reaction mixture was quenched by the addition of saturated NaHCO₃ at −10° C. and extracted with DCM. The combined organics were dried over anhydrous Na₂SO₄ and concentrated in vacuo. The resulting crude product was purified by Prep-HPLC (Water with 10 mmol/L NH₄HCO₃+0.05% NH₃H₂O and MeOHt 58% to 80% over 10 min) to afford 4-(4-bromophenyl)-4-fluoro-1-methylpiperidine (700 mg, 34.74%) as a white solid. LCMS (ESI): m/z 272.00 (M+H⁺).

### Step 3: Int. 186

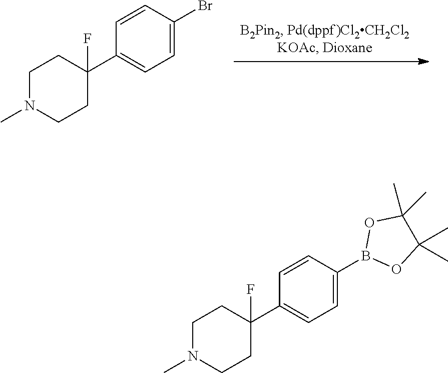

To a mixture of 4-(4-bromophenyl)-4-fluoro-1-methylpiperidine (700 mg, 2.57 mmol, 1 Equiv.) and bis(pinacolato)diboron (979.70 mg, 3.86 mmol, 1.5 Equiv.) in dioxane (20 mL) were added Pd(dppf)Cl₂·CH₂Cl₂ (209.52 mg, 0.257 mmol, 0.1 Equiv.) and KOAc (757.27 mg, 7.72 mmol, 3 Equiv.) at room temperature under nitrogen atmosphere. The resulting mixture was stirred overnight at 80° C. under nitrogen atmosphere. The reaction mixture was diluted with water and extracted with EtOAc. The organic layer was dried over anhydrous Na₂SO₄ and concentrated in vacuo. The residue was purified FCC (DCM/MeOH 10:1) to afford Int. 186 (211 mg, 25.70%) as a light yellow solid after the removal of solvent. LCMS (ESI): m/z 320.25 (M+H⁺). ¹H NMR (400 MHz, DMSO-d₆): δ 1.29 (s, 11H), 1.88-2.01 (m, 3H), 2.09-2.32 (m, 2H), 2.42 (s, 3H), 2.51 (s, 2H), 2.93 (s, 2H), 7.40 (dd, J=21.2, 7.9 Hz, 2H), 7.76 (dd, J=42.1, 7.8 Hz, 2H).

### Int. 187: Preparation of 2-morpholino-5-(4,4,5,5-tetramethyl-1,3,2-dioxaborolan-2-yl)benzonitrile

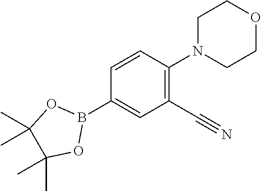 [SMILES: CC1(C)OB(c2ccc(N3CCOCC3)c(C#N)c2)OC1(C)C]

2-morpholino-5-(4,4,5,5-tetramethyl-1,3,2-dioxaborolan-2-yl)benzonitrile (Int. 187): Int. 187 was prepared according to the procedure for step 3 of Int. 172 using 5-Bromo-2-morpholinobenzonitrile as starting material. LCMS (ESI+): m/z 313.85 (M+H).

### Int. 189: Preparation of (2S,4R)-4-fluoro-1-(2,3, 3,3-tetrafluoro-2-methylpropanoyl) pyrrolidine-2-carboxylic acid

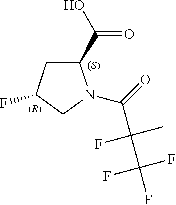

### Step 1: benzyl (2S,4R)-4-fluoro-1-(3, 3,3-trifluoro-2-hydroxy-2-methylpropanoyl) pyrrolidine-2-carboxylate

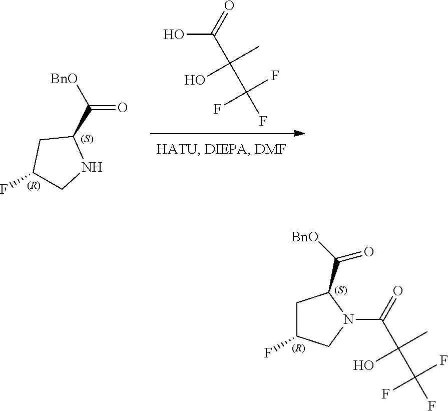

To a solution of benzyl (2S,4R)-4-fluoropyrrolidine-2-carboxylate (4.24 g, 18.9 mmol, 1.0 Equiv.) in DMF (70 mL) was added DIEPA (7.36 g, 56.9 mmol, 9.92 mL, 3.0 Equiv.), then 3,3,3-trifluoro-2-hydroxy-2-methyl-propanoic acid (3.00 g, 18.9 mmol, 1.0 Equiv.) and HATU (10.8 g, 28.4 mmol, 1.5 Equiv.) were added at 0° C. and stirred at 25° C. for 2 h. The reaction mixture was quenched with water (50 mL) and the aqueous phase was extracted with EtOAc (80 ml×3), the combined organic phase was washed with brine (30 mL×2), dried with anhydrous Na₂SO₄, filtered, and concentrated in vacuum. The residue was purified by silica gel column chromatography (Pet. Ether/EtOAc, 1:0 to 3:2) to afford benzyl (2S,4R)-4-fluoro-1-(3, 3,3-trifluoro-2-hydroxy-2-methyl-propanoyl) pyrrolidine-2-carboxylate (6.14 g, 16.9 mmol, 89.0%) as a yellow oil. LCMS (ESI+): m/z 364.3 (M+H⁺). ¹H NMR (400 MHz, CDCl₃-d): 67.39-7.30 (m, 5H), 5.39-5.22 (m, 1H), 5.21-5.10 (m, 2H), 4.76 (dt, J=2.4, 8.8 Hz, 1H), 4.54-4.30 (m, 1H), 3.92-3.70 (m, 1H), 2.84-2.49 (m, 1H), 2.00-1.75 (m, 1H), 1.65 (d, J=5.2 Hz, 3H).

### Step 2: benzyl (2S,4R)-4-fluoro-1-(2,3, 3,3-tetrafluoro-2-methylpropanoyl) pyrrolidine-2-carboxylate

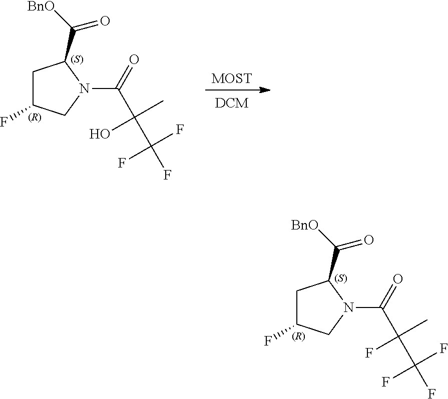

To a solution of benzyl (2S,4R)-4-fluoro-1-(3, 3,3-trifluoro-2-hydroxy-2-methylpropanoyl) pyrrolidine-2-carboxylate (2.57 g, 7.07 mmol, 1.0 Equiv.) in DCM (25 mL) was added trifluoro-(morpholino)-sulfane (4.96 g, 28.3 mmol, 3.45 mL, 4.0 Equiv.) and stirred at 50° C. for 12 hr under N₂. The reaction mixture was poured into ice (50 ml) and the aqueous phase was extracted with DCM (30 ml×3), the combined organic phase was washed with brine (20 mL×2), dried with anhydrous Na₂SO₄, filtered, and concentrated in vacuum. The residue was purified by silica gel column chromatography (Pet. Ether/EtOAc, 1:0 to 3:2) to afford benzyl (2S,4R)-4-fluoro-1-(2,3, 3,3-tetrafluoro-2-methylpropanoyl) pyrrolidine-2-carboxylate (2.40 g, 6.57 mmol, 46.4%) as a white solid. LCMS (ESI+): m/z 366.3 (M+H⁺). ¹H NMR (400 MHz, CDCl₃): δ7.41-7.32 (m, 5H), 5.37-5.26 (m, 1H), 5.23-5.10 (m, 2H), 4.83-4.71 (m, 1H), 4.37-4.22 (m, 1H), 3.99-3.79 (m, 1H), 2.88-2.54 (m, 1H), 2.28-2.06 (m, 1H), 2.04-1.92 (m, 1H), 1.89-1.72 (m, 3H).

### Step 3: Int. 189

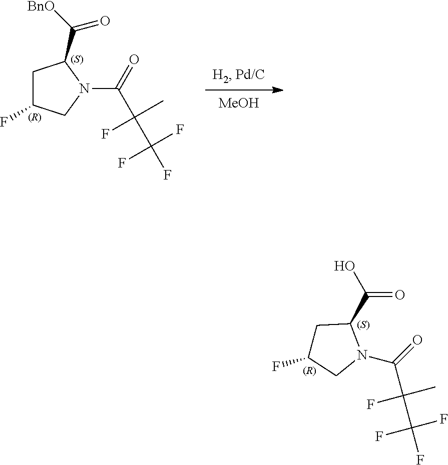

To a solution of benzyl (2S,4R)-4-fluoro-1-(2,3, 3,3-tetrafluoro-2-methylpropanoyl) pyrrolidine-2-carboxylate (2.40 g, 6.57 mmol, 1.0 Equiv.) in MeOH (25 mL) was added 10% Pd/C (500 mg, 469 μmol) and purged with H₂. The mixture was stirred at 25° C. for 2 h under H₂ (25 psi) atmosphere. The reaction solution was filtered over Diatomaceous earth, and the filtrate was concentrated under reduced pressure to afford Int. 189 (1.60 g, 5.81 mmol, 88.5%) as a colorless oil. LCMS (ESI+): m/z 276.3 (M+H⁺). ¹H NMR (400 MHz, CDCl₃): δ8.25 (d, J=1.2 Hz, 1H), 5.45-5.14 (m, 1H), 4.79-4.62 (m, 1H), 4.30 (ddd, J=6.4, 13.2, 20.0 Hz, 1H), 4.00-3.79 (m, 1H), 2.94-2.57 (m, 1H), 2.32-2.04 (m, 1H), 1.90-1.73 (m, 3H).

### Int. 190: 3-(4-(4,4,5,5-tetramethyl-1,3,2-dioxaborolan-2-yl)phenyl)-6,7-dihydro-4H-pyrazolo[5,1-cl[1,4]oxazine

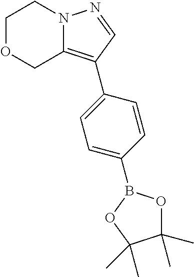 [SMILES: CC1(C)OB(c2ccc(-c3cnn4c3COCC4)cc2)OC1(C)C]

### Step 1: 3-(4-bromophenyl)-6,7-dihydro-4H-pyrazolo[5,1-c][1,4]oxazine

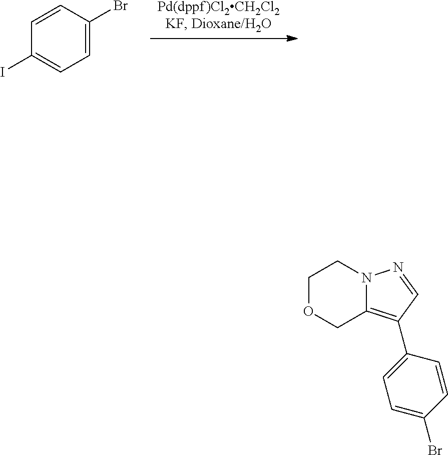 [SMILES: Brc1ccc(I)cc1 ; "Pd(dppf)Cl2·CH2Cl2KF, Dioxane/H2O" ; Brc1ccc(-c2cnn3c2COCC3)cc1]

A solution of 4-bromoiodobenzene (1 g, 3.535 mmol, 1 Equiv.), 3-(4,4,5,5-tetramethyl-1,3,2-dioxaborolan-2-yl)-4H,6H,7H-pyrazolo[3,2-c][1,4]oxazine (0.97 g, 3.889 mmol, 1.1 Equiv.), KF (0.41 g, 7.07 mmol, 2 Equiv.) and Pd(dppf)Cl₂·CH₂Cl₂ (0.23 g, 0.283 mmol, 0.08 Equiv.) in dioxane (10 mL) and water (4 mL) was stirred overnight at 80° C. under nitrogen atmosphere. The residue was diluted with water (20 mL) and extracted with EtOAc (3×50 mL). The combined organics were dried over Na₂SO₄ and concentrated in vacuo. The residue was purified by silica gel column chromatography, eluted with Pet. Ether/EtOAc (50%) to afford 3-(4-bromophenyl)-4H,6H,7H-pyrazolo[3,2-c][1,4]oxazine (750 mg, 76.01%) as a yellow solid. LCMS: (ESI): m/z 279.05 (M+H⁺).

### Step 2: Int. 190

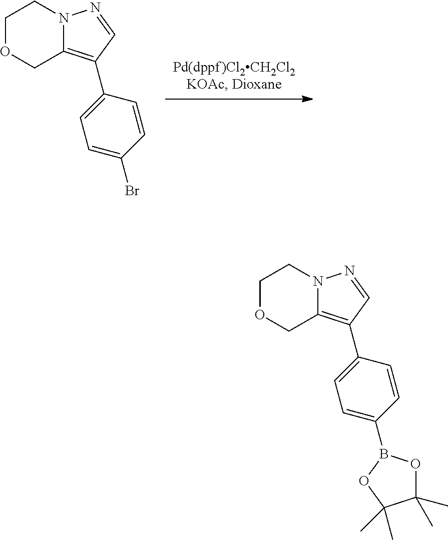 [SMILES: Brc1ccc(-c2cnn3c2COCC3)cc1 ; "Pd(dppf)Cl2·CH2Cl2KOAc, Dioxane" ; CC1(C)OB(c2ccc(-c3cnn4c3COCC4)cc2)OC1(C)C]

A solution of 3-(4-bromophenyl)-4H,6H,7H-pyrazolo[3,2-c][1,4]oxazine (750 mg, 2.687 mmol, 1 Equiv.), bis(pinacolato)diboron (750.53 mg, 2.96 mmol, 1.1 Equiv.), KOAc (527.39 mg, 5.37 mmol, 2 Equiv.) and Pd(dppf)Cl₂·CH₂Cl₂ (175.10 mg, 0.215 mmol, 0.08 Equiv.) in dioxane (10 mL) was stirred for 3 h at 100° C. under nitrogen atmosphere. The residue was purified by Prep-TLC (PE/EtOAc, 50%) to afford Int. 190 (0.7230 g, 82.49%) as a yellow solid. LCMS (ESI): m/z 327.20 (M+H⁺). ¹H NMR (400 MHz, DMSO-d₆): δ 1.07 (s, 3H), 1.16 (s, 4H), 1.30 (s, 10H), 4.21-4.08 (m, 4H), 4.21-4.08 (m, 4H), 7.41-7.33 (m, 2H), 5.03 (s, 2H), 7.71-7.63 (m, 2H), 7.93 (d, J=20.4 Hz, 1H).

### Int. 191: Preparation of 1-methyl-4-(4-(4-(4,4,5,5-tetramethyl-1,3,2-dioxaborolan-2-yl)phenyl)-1H-pyrazol-1-yl)piperidine

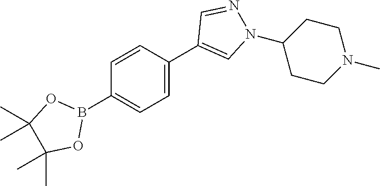

### Step 1: 4-(4-(4-bromophenyl)-1H-pyrazol-1-yl)-1-methylpiperidine

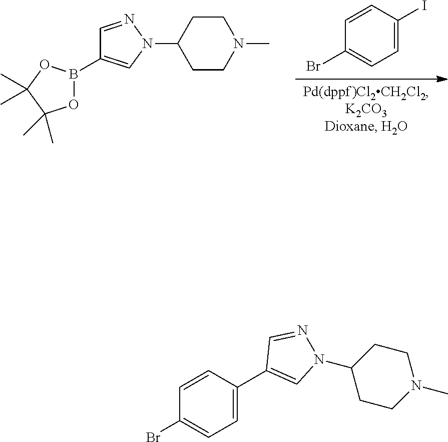

To a solution of 1-bromo-4-iodobenzene (5.13 g, 18.13 mmol, 1.1 Equiv.) in dioxane (60 mL) was added 1-methyl-4-(4-(4,4,5,5-tetramethyl-1,3,2-dioxaborolan-2-yl)-1H-pyrazol-1-yl)piperidine (4.8 g, 16.48 mmol, 1 Equiv.), K₂CO₃ (4.56 g, 32.97 mmol, 2 Equiv.) in H₂O (15 mL) and Pd(dppf)Cl₂·CH₂Cl₂ (1.35 g, 1.65 mmol, 0.1 Equiv.) under N₂. The reaction stirred at 90° C. for 16 hrs under N₂. LCMS showed starting material was consumed completely and desired mass was detected. The reaction mixture was cooled to room temperature and quenched by water (100 mL), extracted with EtOAc (2×60 mL). The combined organics were washed with brine (100 mL), dried over Na₂SO₄, filtered, and concentrated under reduced pressure to give a crude residue. The residue was purified by column chromatography (DCM/methanol, 20:1) to afford crude product. The product was triturated with MTBE (50 mL) and filtered. The filter cake was dried and collected to give 4-(4-(4-bromophenyl)-1H-pyrazol-1-yl)-1-methylpiperidine (2.36 g, 4.79 mmol, 29.1%) as a light yellow solid. LCMS (ESI+): m/z 320.2 (M+H⁺). ¹H NMR (400 MHz, MeOD-d₄) δ=8.09 (s, 1H), 7.83 (s, 1H), 7.48 (s, 4H), 4.19 (dt, J=5.5, 10.6 Hz, 1H), 3.01 (br d, J=11.9 Hz, 2H), 2.34 (s, 3H), 2.26-2.21 (m, 2H), 2.15-2.08 (m, 4H)

### Step 2: Int. 191

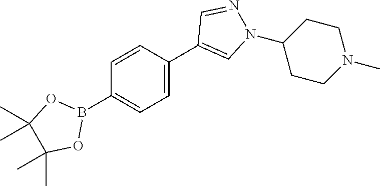

The boronic ester, Int. 191, was synthesized using the procedure outlined in Step 2 of the synthesis of Int. 179 using 4-(4-(4-bromophenyl)-1H-pyrazol-1-yl)-1-methylpiperidine as starting material. LCMS (ESI+): m/z 368.3 (M+H⁺). ¹H NMR (400 MHz, CDCl₃) δ=7.86-7.76 (m, 3H), 7.74 (s, 1H), 7.49 (d, J=8.1 Hz, 2H), 4.26-4.13 (m, 1H), 3.08 (br d, J=11.9 Hz, 2H), 2.39 (s, 3H), 2.32-2.10 (m, 6H), 1.36 (s, 12H)

### Int. 192: Preparation of (2R,6R)-2,6-dimethyl-4-(4-(4,4,5,5-tetramethyl-1,3,2-dioxaborolan-2-yl)phenyl)morpholine

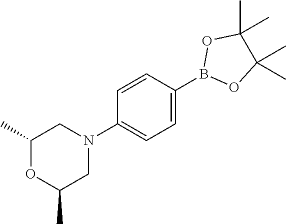

### Step 1: (2R,6R)-2,6-dimethyl-4-phenylmorpholine

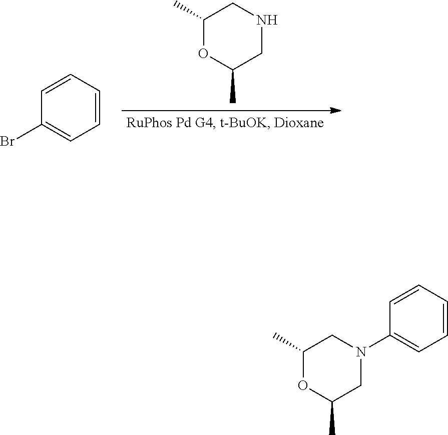

To a solution of bromobenzene (2 g, 12.74 mmol, 1.34 mL, 1 Equiv.) in dioxane (30 mL) was added (2R,6R)-2,6-dimethylmorpholine (1.61 g, 14.01 mmol, 1.1 Equiv.), t-BuONa (2.45 g, 25.48 mmol, 2 Equiv.) and RuPhos Pd G4 (1.08 g, 1.27 mmol, 0.1 Equiv.) under N₂. The mixture was stirred at 90° C. for 2 hrs under N₂. LCMS showed starting material was consumed and desired mass was detected. The residue was diluted with H₂O (50 mL) and extracted with EtOAc (3×50 mL). The combined organic layers were washed with brine (50 mL), dried over anhydrous Na₂SO₄, then filtered and concentrated under reduced pressure to give a crude residue. The residue was purified by column chromatography (Pet. Ether/EtOAc, 10:1) to afford (2R,6R)-2,6-dimethyl-4-phenyl-morpholine (1.7 g, 8.90 mmol, 64%) as a yellow oil. LCMS (ESI+): m/z 192.10 (M+H⁺). ¹H NMR (400 MHz, CDCl₃): δ 7.26-7.13 (m, 2H), 6.89-6.71 (m, 3H), 4.09 (d, 2H), 3.14 (dd, 2H), 2.82 (dd, 2H), 1.25 (d, 6H)).

### Step 2: (2R,6R)-4-(4-bromophenyl)-2,6-dimethylmorpholine

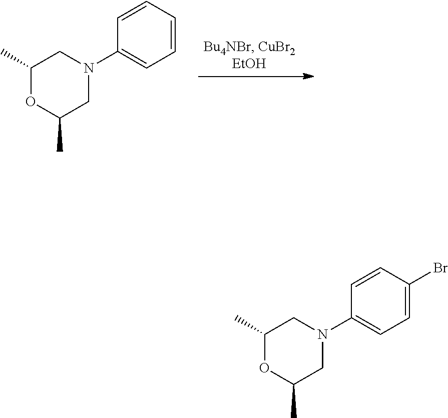

To a solution of (2R,6R)-2,6-dimethyl-4-phenyl-morpholine (1.5 g, 7.84 mmol, 1 Equiv.) in EtOH (30 mL) was added tetrabutylammoniumbromide (2.53 g, 7.84 mmol, 1 Equiv.) and CuBr₂ (2.63 g, 11.76 mmol, 1.5 Equiv.) under N₂. The mixture was stirred at 25° C. for 12 hr. LCMS showed starting material was consumed and desired mass was detected. The resulting mixture was filtered and washed with EtOAc (100 mL). The filtrate was concentrated under reduced pressure to remove EtOH, then diluted with H₂O (50 mL) and extracted with EtOAc (3×50 mL). The combined organic layers were washed with brine (50 mL), dried over anhydrous Na₂SO₄, then filtered and concentrated under reduced pressure to give a crude residue. The residue was purified by column chromatography (Pet. Ether/EtOAc, 10:1) to afford (2R,6R)-4-(4-bromophenyl)-2,6-dimethylmorpholine (1.5 g, 5.6 mmol, 71%) as a pale-yellow solid. LCMS (ESI+): m/z 270.0 (M+H⁺). ¹H NMR (400 MHz, CDCl₃): δ 7.45-7.29 (m, 2H), 6.87-6.62 (m, 2H), 4.16 (d, 2H), 3.18 (dd, 2H), 2.86 (dd, 2H), 1.31 (d, 6H)

### Step 3: Int. 192

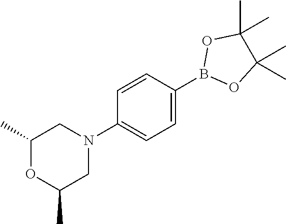

The boronic ester, Int. 192, was synthesized using the procedure outlined in Step 2 of the synthesis of Int. 179 using (2R,6R)-4-(4-bromophenyl)-2,6-dimethylmorpholine as the starting material. LCMS (ESI+): m/z 318.20 (M+H⁺). ¹H NMR (400 MHz, CDCl₃): δ 7.72 (d, 2H), 6.85 (d, 2H), 4.26-4.03 (m, 2H), 3.30 (dd, 2H), 2.98 (dd, 2H), 1.37-1.28 (m, 18H)

### Int. 193: Preparation of tert-butyl 3-(4-(4-(4,4,5,5-tetramethyl-1,3,2-dioxaborolan-2-yl)phenyl)piperazin-1-yl)azetidine-1-carboxylate

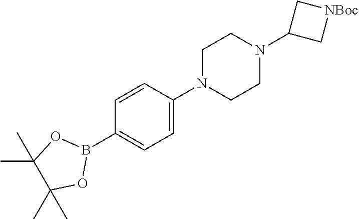 [SMILES: CC(C)(C)OC(=O)N1CC(N2CCN(c3ccc(B4OC(C)(C)C(C)(C)O4)cc3)CC2)C1]

Int. 193 was synthesized following the procedure for Int. 172 (step 2) using 1-(4-(4,4,5,5-tetramethyl-1,3,2-dioxaborolan-2-yl)phenyl)piperazine and tert-butyl-3-oxoazetidine-1-carboxylate as starting materials. LCMS (ESI+): m/z 444.53 (M+H).

### Int. 195: N-ethylhex-5-en-1-amine

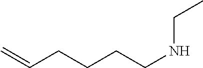

### Step 1: Isopropyl 1-formyl-3,3-dimethoxycyclobutane-1-carboxylate

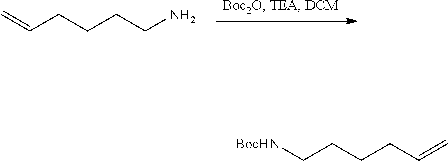

Into a solution of hex-5-en-1-amine (500 mg, 5.041 mmol, 1 Equiv.) and TEA (1.53 g, 15.123 mmol, 3 Equiv.) in DCM (70 mL) was added Boc₂O (1.32 g, 6.049 mmol, 1.2 Equiv.) dropwise at 0° C. under nitrogen atmosphere. The resulting solution was stirred for 16 hr at room temperature. The mixture was concentrated in vacuo. The residue was purified by silica gel column chromatography, eluted with Pet. Ether/EtOAc (50:1) to afford tert-butyl N-(hex-5-en-1-yl)carbamate (176.5 mg, 17.42%) as a colorless oil. LCMS: (ESI): m/z 200.16 (M+H⁺). ¹H NMR (400 MHz, DMSO-d₆): δ 1.37 (s, 13H), 1.94-2.17 (m, 2H), 2.90 (q, J=6.4, 6.4, 6.5 Hz, 2H), 4.81-5.11 (m, 2H), 5.78 (ddt, J=6.6, 6.6, 10.2, 16.9 Hz, 1H), 6.78 (t, J=5.6, 5.6 Hz, 1H).

### Step 2: Tert-butyl ethyl(hex-5-en-1-yl)carbamate

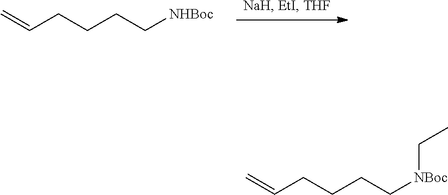

Into a solution of tert-butyl N-(hex-5-en-1-yl)carbamate (4.2 g, 21.07 mmol, 1 Equiv.) in THF (40 mL) was added portion wise NaH (1.01 g, 25.29 mmol, 1.2 Equiv., 60%) at 0° C. under nitrogen atmosphere. The mixture was stirred for 30 min at 0° C. Iodoethane (4.93 g, 31.61 mmol, 1.5 Equiv.) was added dropwise. The mixture was stirred for 16 hr at room temperature. The reaction was quenched by the addition of sat. NH₄Cl (aq.) (100 mL) at 0° C. and extracted with EtOAc (3×50 mL). The combined organic layers were washed with brine (2×50 mL) and dried over anhydrous MgSO₄. After filtration, the filtrate was concentrated under reduced pressure. The residue was purified by silica gel column chromatography, eluted with Pet. Ether/EtOAc (50:1) to afford tert-butyl ethyl(hex-5-en-1-yl)carbamate (3.04 g, 11.35 mmol, 53.9%) as a yellow oil. LCMS (ESI): m/z 228.35 (M+H⁺). ¹H NMR (400 MHz, DMSO-d₆): δ 1.01 (t, J=7.0, 7.0 Hz, 3H), 1.26-1.34 (m, 2H), 1.38 (s, 9H), 1.45 (t, J=7.4, 7.4 Hz, 2H), 2.02 (p, J=6.7, 6.7, 6.9, 6.9 Hz, 2H), 3.06-3.18 (m, 4H), 4.85-5.09 (m, 2H), 5.67-5.94 (m, 1H).

### Step 3: Int 195

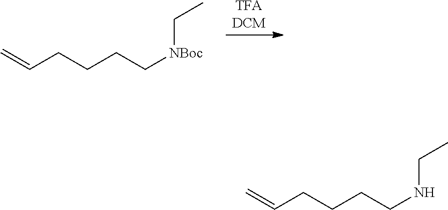

Tert-butyl ethyl(hex-5-en-1-yl)carbamate was dissolved in TFA:DCM (1:1, 0.1M) and stirred at RT until the complete consumption of starting material was observed by LCMS. The solvent was removed and the residue was co-evaporated with DCE twice. The crude deprotected amine was used in the next reaction without further purification. LCMS (ESI): m/z 128.35 (M+H⁺).

### Int. 196: Preparation of (2R,6S)-1,2,6-trimethyl-4-(4-(4,4,5,5-tetramethyl-1,3,2-dioxaborolan-2-yl)phenyl)piperazine

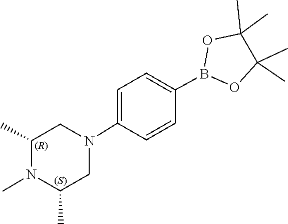

### Step 1: (2R,6S)-4-(4-bromophenyl)-1,2,6-trimethylpiperazine

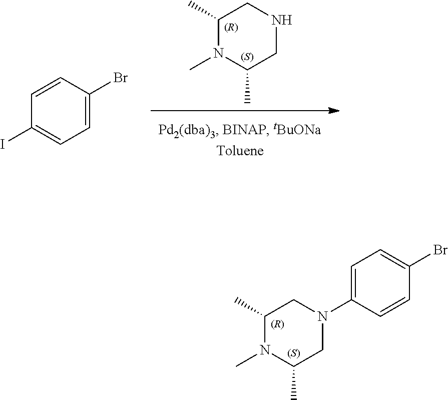

A mixture of 1-bromo-4-iodo-benzene (5.00 g, 17.6 mmol, 1.0 Equiv.), (2S,6R)-1,2,6-trimethylpiperazine (2.27 g, 17.6 mmol, 1.0 Equiv.), ^{t}BuONa (5.10 g, 53.0 mmol, 3.0 Equiv.), BINAP (110 mg, 176 μmol, 0.01 Equiv.) and Pd₂(dba)₃ (161 mg, 176 μmol, 0.01 Equiv.) in toluene (50 mL) was degassed and purged with N₂ for 3 times, and then the mixture was stirred at 110° C. for 6 hr under N₂ atmosphere. The reaction mixture was concentrated to remove toluene, then water (50 mL) was added and the aqueous phase was extracted with EtOAc (50 mL×3), the combined organic layers was washed with brine (50 mL), dried over Na₂SO₄, filtered and the filtrate was concentrated to give a residue. The residue was purified by prep-HPLC (TFA condition; column: Phenomenex luna C18 (250×70 mm, 15 um); mobile phase: [H₂O (0.1% TFA)-ACN]; gradient: 15%-45% B over 20.0 min) to give (2S,6R)-4-(4-bromophenyl)-1,2,6-trimethyl-piperazine (1.8 g, 6.29 mmol, 35.6%) as a brown solid. LCMS (ESI+): m/z 283.2/285.2 (M+H⁺).

### Step 2: Int. 196

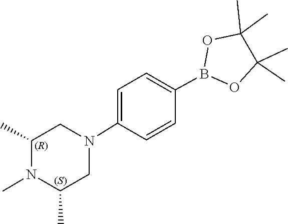

The boronic ester, Int. 196, was synthesized using the procedure outlined in Step 2 of the synthesis of Int. 179 using (2S,6R)-4-(4-bromophenyl)-1,2,6-trimethyl-piperazine as starting material LCMS (ESI+): m/z 331.4 (M+H⁺). ¹H NMR (400 MHz, DMSO): δ7.50 (d, J=8.0 Hz, 1H), 6.91 (d, J=8.0 Hz, 1H), 3.71-3.69 (m, 1H), 2.34-2.32 (m, 4H), 2.27 (s, 1H), 1.17 (s, 12H), 1.12 (s, 6H).

### Int. 199: Preparation of 1-(oxetan-3-yl)-4-(4-(4,4,5,5-tetramethyl-1,3,2-dioxaborolan-2-yl)phenyl)piperazine

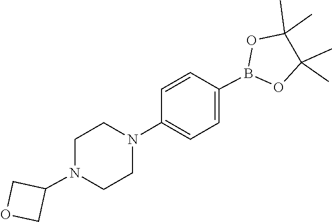 [SMILES: CC1(C)OB(c2ccc(N3CCN(C4COC4)CC3)cc2)OC1(C)C]

1-(oxetan-3-yl)-4-(4-(4,4,5,5-tetramethyl-1,3,2-dioxaborolan-2-yl)phenyl)piperazine (Int. 199) was prepared according to the procedure for Int. 193 using Oxentan-3-on as starting material. LCMS (ESI+): m/z 344.39 (M+H).

### Int. 400: Preparation of (3aR,6aS)-5-(4-(4,4,5,5-tetramethyl-1,3,2-dioxaborolan-2-yl)phenyl)hexahydro-1H-furo[3,4-c]pyrrole

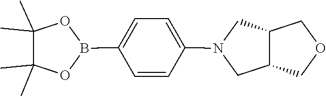 [SMILES: CC1(C)OB(c2ccc(N3C[C@H]4COC[C@H]4C3)cc2)OC1(C)C]

### Step 1: (3aR,6aS)-5-(4-bromophenyl)hexahydro-1H-furo[3,4-c]pyrrole

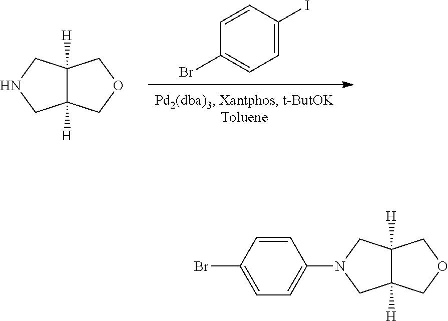 [SMILES: C1NC[C@H]2COC[C@@H]12 ; Brc1ccc(I)cc1 ; Brc1ccc(N2C[C@H]3COC[C@H]3C2)cc1]

In a sealed tube were placed (3aR,6aS)-hexahydro-1H-furo[3,4-c]pyrrole (109 mg, 1 Equiv., 964 μmol), 1-bromo-4-iodobenzene (300 mg, 1.1 Equiv., 1.06 mmol) and t-BuOK (216 mg, 2 Equiv., 1.93 mmol). Toluene (10.6 mL) was added, and the reaction was degassed for 15 min with Ar. Then, 4,5-Bis(diphenylphosphino)-9,9-dimethyl xanthene (83.7 mg, 0.15 Equiv., 145 μmol) and Tris(dibezylideneacetone)dipalladium (44.1 mg, 0.05 Equiv., 48.2 mol) were added. The reaction was heated to 45° C. until the complete consumption of starting material (monitored by LCMS). Then it was cooled to room temperature, quenched with water and the organic layers were extracted with EtOAc (3×10 mL). The combined organic layers were washed with water (2×10 mL) and brine (10 mL), dried (Na₂SO₄), concentrated, and purified by flash column chromatography (Hexane:EtOAc 2:3) and concentrated. This resulted in (3aR,6aS)-5-(4-bromophenyl)hexahydro-1H-furo[3,4-c]pyrrole (243 mg, 906 μmol, 94.0%) as yellow solid. LCMS (ESI+): m/z 269.35 (M+H).

### Step 2: Int. 400

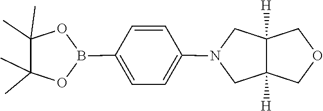 [SMILES: CC1(C)OB(c2ccc(N3C[C@H]4COC[C@H]4C3)cc2)OC1(C)C]

(3aR,6aS)-5-(4-(4,4,5,5-tetramethyl-1,3,2-dioxaborolan-2-yl)phenyl)hexahydro-1H-furo[3,4-c]pyrrole (Int. 400) was prepared according to the procedure for step 3 of synthesis of Int. 172 using (3aR,6aS)-5-(4-bromophenyl)hexahydro-1H-furo[3,4-c]pyrrole as starting material. LCMS (ESI+): m/z 315.34 (M+H).

### Int. 401: Preparation of (R)-8-(4-(4,4,5,5-tetramethyl-1,3,2-dioxaborolan-2-yl)phenyl)octahydropyrazino[2,1-c][1,4]oxazine

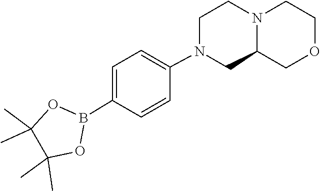 [SMILES: CC1(C)OB(c2ccc(N3CCN4CCOC[C@H]4C3)cc2)OC1(C)C]

### Step 1: (R)-8-(4-bromophenyl)octahydropyrazino[2,1-c][1,4]oxazine

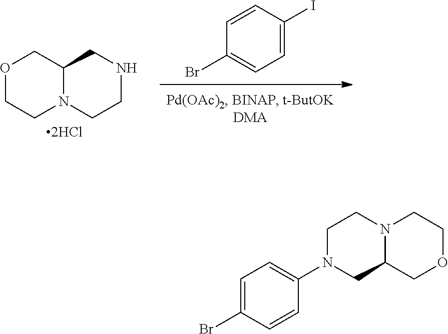 [SMILES: C1CN2CCOC[C@H]2CN1 ; Brc1ccc(I)cc1 ; Brc1ccc(N2CCN3CCOC[C@H]3C2)cc1]

In a sealed tube were placed (R)-octahydropyrazino[2,1-c][1,4]oxazine dihydrochloride (351 mg, 1 Equiv., 1.63 mmol), 1-bromo-4-iodobenzene (600 mg, 1.3 Equiv., 2.12 mmol) t-BuOK (915 mg, 1.03 mL, 5 Equiv., 8.16 mmol) in DMA (10.6 mL). The reaction was degassed for 15 min with Ar and then Pd(OAc)₂ (18.3 mg, 0.05 Equiv., 81.6 mol) and BINAP (102 mg, 0.1 Equiv., 163 μmol) were added. The reaction was heated to 70° C. and stirred until the complete consumption of starting material (monitored by LCMS). The reaction was cooled to room temperature, quenched with water and extracted with EtOAc (3×10 mL) The combined organic layers were washed with water (2×10 mL) and brine (10 mL), dried with Na₂SO₄, concentrated, and purified by flash column chromatography (DCM/MeOH, 9:1) and concentrated. This resulted in (R)-8-(4-bromophenyl)octahydropyrazino[2,1-c][1,4]oxazine (50 mg, 0.17 mmol, 10%) as yellow solid. LCMS (ESI+): m/z 297.08 (M+H).

### Step 2: Int. 401

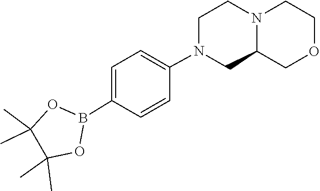 [SMILES: CC1(C)OB(c2ccc(N3CCN4CCOC[C@H]4C3)cc2)OC1(C)C]

(R)-8-(4-(4,4,5,5-tetramethyl-1,3,2-dioxaborolan-2-yl)phenyl)octahydropyrazino[2,1-c][1,4]oxazine (Int. 401) was prepared according to the procedure for step 3 of Int. 172 using (R)-8-(4-bromophenyl)octahydropyrazino[2,1-c][1,4]oxazine as starting material. LCMS (ESI+): m/z 344.23 (M+H).

### Int. 403: Preparation of tert-butyl 3-(4-(4-(4,4,5,5-tetramethyl-1,3,2-dioxaborolan-2-yl)phenyl)piperazin-1-yl)azetidine-1-carboxylate

### Step 1: Int. 403

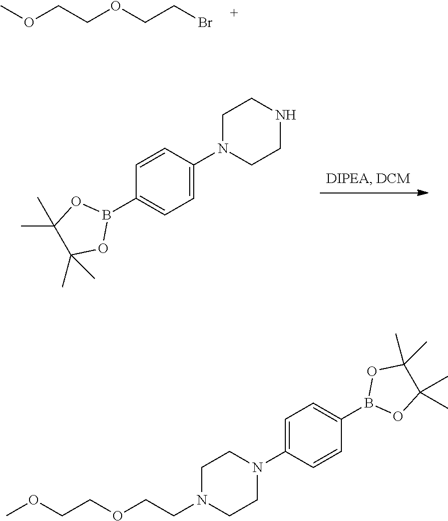

1-(4-(4,4,5,5-tetramethyl-1,3,2-dioxaborolan-2-yl)phenyl)piperazine (200 mg, 1 Equiv., 694 μmol), DIPEA (179 mg, 242 μL, 2 Equiv., 1.39 mmol), and 2-(2-Methoxyethoxy)ethylbromide (254 mg, 188 μL, 2 Equiv., 1.39 mmol) were dissolved in DCM (2.0 mL) and stirred until the starting material was completely consumed (monitored by LCMS). The reaction mixture was diluted with water (10 mL) and extracted with EtOAc (2×10 mL). The combined organic layer was washed with brine, dried over MgSO₄, filtered, and concentrated. The crude mixture was purified by silica gel column chromatography (DCM/MeOH, 95:5) and concentrated. This afforded Int. 403 (230 mg, 589 μmol, 84.9%) as yellow solid. LCMS (ESI+): m/z 390.67 (M+H).

### Int. 404: Preparation of 1-(2,2-difluoroethyl)-4-(4-(4,4,5,5-tetramethyl-1,3,2-dioxaborolan-2-yl)phenyl)piperazine

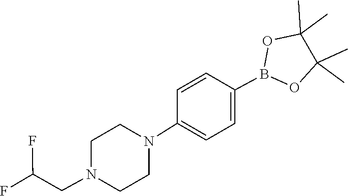 [SMILES: CC1(C)OB(c2ccc(N3CCN(CC(F)F)CC3)cc2)OC1(C)C]

1-(2,2-difluoroethyl)-4-(4-(4,4,5,5-tetramethyl-1,3,2-dioxaborolan-2-yl)phenyl)piperazine (Int. 402) was prepared according to the procedure for Int. 403 using 2,2-difluoroethyl trifluoromethanesulfonate as starting material. LCMS (ESI+): m/z 352.02 (M+H).

### Int. 405: Preparation of 1-((1-methyl-1H-pyrazol-4-yl)methyl)-4-(4-(4,4,5,5-tetramethyl-1,3,2-dioxaborolan-2-yl)phenyl)piperazine

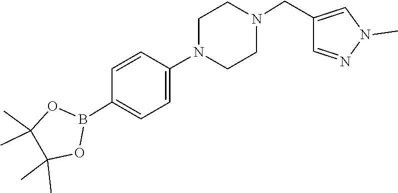

1-((1-methyl-1H-pyrazol-4-yl)methyl)-4-(4-(4,4,5,5-tetramethyl-1,3,2-dioxaborolan-2-yl)phenyl)piperazine (Int. 405) was prepared according to the procedure for Int. 193 using 1-methyl-1H-pyrazole-4-carbaldehyde as starting material. LCMS (ESI+): m/z 383.16 (M+H).

### Int. 406: Preparation of (S)-2-((tert-butoxycarbonyl)(methyl)amino)-4-methoxy-4-oxobutanoic acid

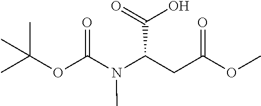 [SMILES: COC(=O)C[C@@H](C(=O)O)N(C)C(=O)OC(C)(C)C]

### Step 1: (9H-fluoren-9-yl)methyl(S)-4-(2-methoxy-2-oxoethyl)-5-oxooxazolidine-3-carboxylate

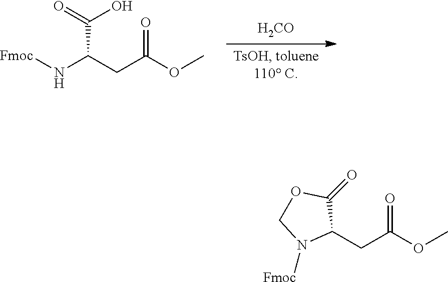

A mixture of (S)-2-((((9H-fluoren-9-yl)methoxy)carbonyl)amino)-4-methoxy-4-oxobutanoic acid (100 g, 270.74 mmol, 1 eq), 37% formaldehyde (43 g, 1432.1 mmol, 39.44 mL, 5.29 eq), TsOH (2.34 g, 13.54 mmol, 0.05 eq) in toluene (1800 mL) was degassed for several minutes, and then the mixture was stirred at 110° C. for 12 h under N₂ atmosphere. The resultant mixture was filtered over a pad of diatomaceous earth and the filter cake was washed with EtOAc (200 mL×3). The combined filtrates were concentrated under reduced pressure to give a crude residue. The residue was adjusted to pH ˜9 with a saturated Na₂CO₃ solution. The aqueous phase was diluted with additional H₂O (600 mL) and extracted with EtOAc (300 mL×3). The combined organic layers were dried over Na₂SO₄, filtered and concentrated to give (9H-fluoren-9-yl)methyl(S)-4-(2-methoxy-2-oxoethyl)-5-oxooxazolidine-3-carboxylate (90 g, 207.20 mmol, 76.53%) as a yellow oil. The product was used with no further purification. LCMS (ESI+): m/z 382.2 (M+H⁺), ¹H NMR (400 MHz, DMSO-d₆): δ 7.89 (d, J=7.2 Hz, 2H), 7.68-7.61 (m, 2H), 7.46-7.39 (m, 2H), 7.37-7.31 (m, 2H), 5.34 (br d, J=2.0 Hz, 1H), 5.24-5.01 (m, 1H), 4.75-4.18 (m, 4H), 3.60 (s, 3H), 3.19-2.51 (m, 2H).

### Step 2: (S)-2-((((9H-fluoren-9-yl)methoxy)carbonyl)(methyl)amino)-4-methoxy-4-oxobutanoic acid

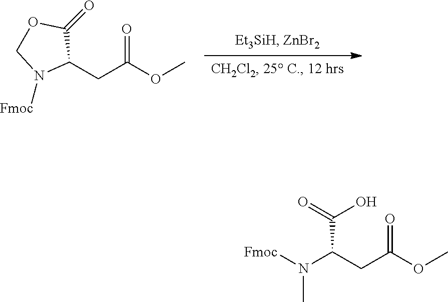

To a solution of (9H-fluoren-9-yl)methyl(S)-4-(2-methoxy-2-oxoethyl)-5-oxooxazolidine-3-carboxylate (85 g, 195.68 mmol, 1 eq) in CH₂Cl₂ (1700 mL) was added ZnBr₂ (66.10 g, 293.52 mmol, 14.68 mL, 1.5 eq) and Et₃SiH (68.26 g, 587.06 mmol, 93.76 mL, 3 eq). The mixture was stirred at 25° C. for 12 h. The resulting mixture was filtered over a pad of diatomaceous earth and the filter cake was washed with additional CH₂Cl₂ (2×200 mL). The filtrate was diluted with H₂O (500 mL) and extracted with EtOAc (200 mL×3). The combined organic layers were dried over Na₂SO₄, filtered and concentrated to give (S)-2-((((9H-fluoren-9-yl)methoxy)carbonyl)(methyl)amino)-4-methoxy-4-oxobutanoic acid (100 g, 194.58 mmol, 99.43%) as a yellow oil. The product was used with no further purification. LCMS (ESI+): m/z 384.1 (M+H⁺), ¹H NMR (400 MHz, DMSO-d₆): δ 13.42 (s, 1H), 7.93-7.85 (m, 2H), 7.69-7.60 (m, 2H), 7.45-7.38 (m, 2H), 7.37-7.28 (m, 2H), 4.93-4.70 (m, 1H), 4.39-4.21 (m, 3H), 3.65-3.55 (m, 3H), 3.00-2.88 (m, 1H), 2.85-2.76 (m, 3H), 2.76-2.57 (m, 1H).

### Step 3: 1-benzyl 4-methyl N-(((9H-fluoren-9-yl)methoxy)carbonyl)-N-methyl-L-aspartate

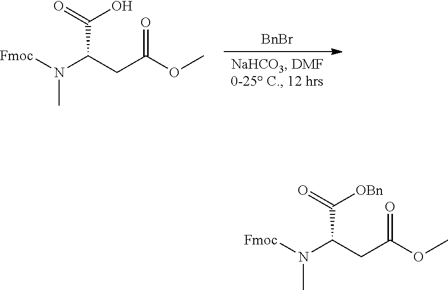

To a solution of (S)-2-((((9H-fluoren-9-yl)methoxy)carbonyl)(methyl)amino)-4-methoxy-4-oxobutanoic acid (80 g, 208.66 mmol, 1 eq) in DMF (700 mL) was added NaHCO₃ (35.06 g, 417.32 mmol, 16.24 mL, 2 eq) at 25° C. for 30 min and BnBr (107.06 g, 625.98 mmol, 74.36 mL, 3 eq) was added at 0° C. The mixture was stirred at 0° C. for 12 h. Then, the mixture was quenched by the addition of H₂O (1500 mL) and extracted with EtOAc (500 mL×3). The combined organic layers were washed with brine (200 mL×3) and dried over Na₂SO₄ and filtered. The filtrate was concentrated and the crude product was purified by column chromatography (SiO₂, pet. ether/EtOAc, 7:1 to 3:1) to give 1-benzyl 4-methyl N-(((9H-fluoren-9-yl)methoxy)carbonyl)-N-methyl-L-aspartate (75 g, 142.55 mmol, 68.32%) as a yellow oil. LCMS (ESI+): m/z 474.4 (M+H⁺), ¹H NMR (400 MHz, DMSO-d₆): δ 7.92-7.83 (m, 2H), 7.64-7.56 (m, 2H), 7.45-7.37 (m, 2H), 7.37-7.31 (m, 2H), 7.31-7.22 (m, 5H), 5.20-4.96 (m, 2H), 4.89-4.75 (m, 1H), 4.46-4.16 (m, 3H), 3.58 (s, 3H), 3.01 (dd, J=6.8, 16.4 Hz, 1H), 2.87-2.73 (m, 4H)

### Step 4: 1-benzyl 4-methyl methyl-L-aspartate

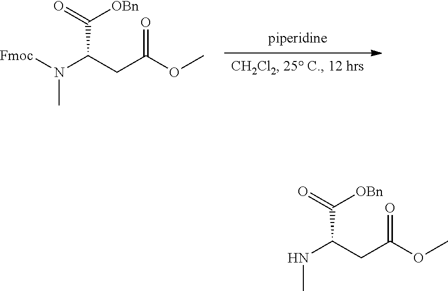

To a solution of 1-benzyl 4-methyl N-(((9H-fluoren-9-yl)methoxy)carbonyl)-N-methyl-L-aspartate (75 g, 158.38 mmol, 1 eq) in CH₂Cl₂ (750 mL) was added piperidine (40.46 g, 547.16 mmol, 46.92 mL, 3 eq) at 0° C. The mixture was stirred at 25° C. for 12 h. The reaction mixture was concentrated. H₂O (1000 mL) was added to the crude product and the aqueous layer was extracted with EtOAc (500 mL×3). The combined organic layers were dried over Na₂SO₄, filtered, and the solution was concentrated to give a crude residue. 1-benzyl 4-methyl methyl-L-aspartate (32 g, 113.98 mmol, 71.96%) was isolated as a yellow oil after column chromatography (SiO₂, pet. ether/EtOAc, 20:1 to 3:1). LCMS (ESI+): m/z 252.3 (M+H⁺), ¹H NMR (400 MHz, DMSO-d₆): δ 7.40-7.31 (m, 5H), 5.19 (s, 2H), 3.61-3.52 (s, 3H), 3.52-3.29 (m, 1H), 2.83-2.51 (m, 2H), 2.28-2.15 (s, 3H).

### Step 5: 1-benzyl 4-methyl N-(tert-butoxycarbonyl)-N-methyl-L-aspartate

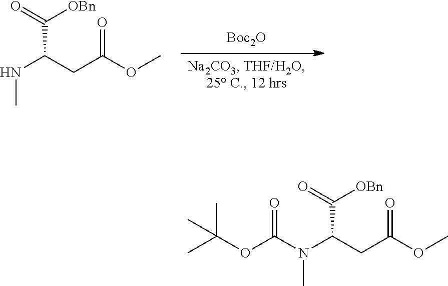 [SMILES: "Boc2O" ; CN[C@@H](CC(=O)OC)C(=O)OCc1ccccc1 ; "Na2CO3, THF/H2O,25° C., 12 hrs" ; COC(=O)C[C@@H](C(=O)OCc1ccccc1)N(C)C(=O)OC(C)(C)C]

To a solution of 1-benzyl 4-methyl methyl-L-aspartate (32 g, 127.34 mmol, 1 eq) in THF (320 mL) and H₂O (320 mL) was added Na₂CO₃ (27 g, 254.7 mmol, 2 eq) and Boc₂O (111.18 g, 509.4 mmol, 117.02 mL, 4 eq). The mixture was stirred at 25° C. for 12 h. The reaction mix was then extracted with EtOAc (300 mL×3). The combined organic layers were washed with brine, dried over Na₂SO₄, filtered, and the filtrate was concentrated to give a crude residue. The residue was purified by column chromatography (SiO₂, Pet. ether/EtOAc, 20:1 to 3:1). The product was then purified by prep-HPLC to give 1-benzyl 4-methyl N-(tert-butoxycarbonyl)-N-methyl-L-aspartate (27 g, 74.92 mmol, 58.83%) as a yellow oil. LCMS (ESI+): m/z 374.3 (M+Na⁺), ¹H NMR (400 MHz, DMSO-d₆): δ 7.36 (d, J=5.6 Hz, 5H), 5.18-5.07 (m, 2H), 4.82-4.59 (m, 1H), 3.59 (d, J=6.0 Hz, 3H), 3.05-2.97 (m, 1H), 2.86-2.76 (m, 4H), 1.38-1.27 (m, 9H).

### Step 6: Int. 406

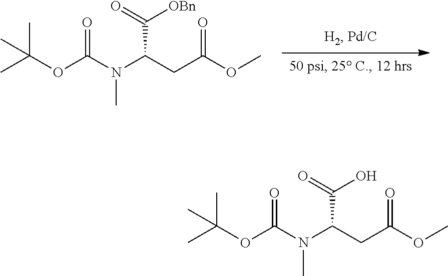 [SMILES: COC(=O)C[C@@H](C(=O)OCc1ccccc1)N(C)C(=O)OC(C)(C)C ; "50 psi, 25° C., 12 hrs" ; "H2, Pd/C" ; COC(=O)C[C@@H](C(=O)O)N(C)C(=O)OC(C)(C)C]

To a solution of 1-benzyl 4-methyl N-(tert-butoxycarbonyl)-N-methyl-L-aspartate (27 g, 76.84 mmol, 1 eq) in MeOH (400 mL) was added Pd/C (8.18 g, 7.68 mmol, 10 Wt.%, 0.1 eq) under N₂ atmosphere. The suspension was degassed and purged with H₂ (×3). The mixture was stirred under H₂ (50 Psi) at 25° C. for 12 h. Upon completion, the reaction was filtered over a pad of diatomaceous earth and the filter cake was washed with EtOAc (100 mL×2). Int. 406 (19.4 g, 67.50 mmol, 87.84%) was isolated as white solid after the removal of solvent. LCMS (ESI+): m/z 284 (M+Na⁺). ¹H NMR (400 MHz, DMSO-d₆): δ 12.88 (br s, 1H), 4.75-4.39 (m, 1H), 3.60 (d, J=6.8 Hz, 3H), 2.98-2.88 (m, 1H), 2.80-2.66 (m, 4H), 1.37 (d, J=15.2 Hz, 9H).

### Int. 408: Preparation of (S)-2-((tert-butoxycarbonyl)(methyl-d3)amino)pent-4-enoic acid

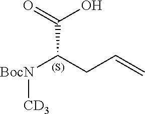

### Step 1: Int. 408

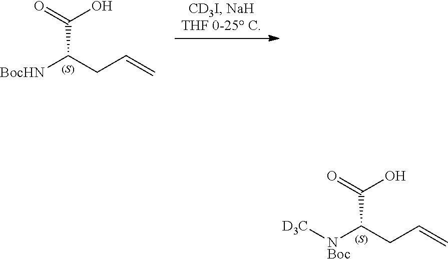

To a solution of(S)-2-((tert-butoxycarbonyl)amino) pent-4-enoic acid (17.5 g, 81.30 mmol, 1 eq) in THF (800 mL) was added 60% NaH (9.76 g, 243.91 mmol, 3 eq) in portions at 0° C. under a nitrogen atmosphere, and the mixture was stirred at 0° C. for 30 min. Then iodomethane-d₃ (82.50 g, 569.12 mmol, 35.42 mL, 7 eq) was added at 0° C., then the reaction was warmed to 25° C. and stirred for 4 h The reaction mixture was cooled to 0° C., quenched with saturated NH₄Cl (300 mL). The reaction mixture was extracted with EtOAc (3×200 mL). The combined organic layers were washed with brine (300 mL), dried over Na₂SO₄, filtered, and concentrated under reduced pressure to give a thick oil. The residue was purified by column chromatography (SiO₂, pet. ether/EtOAc. 10-25%) to give a pale yellow solid. The pale-yellow solid was triturated with pet. ether (45 mL) for 30 min, and the mixture was filtered to give Int. 408 (28 g, 117.22 mmol, 72.09%) as a white solid. LCMS (ESI+): m/z 231.1 (M−H⁻). ¹H NMR (400 MHZ, DMSO-d₆): δ12.73 (s, 1H), 5.81-5.62 (m, 1H), 5.20-4.93 (m, 2H), 4.62-4.30 (m, 1H), 2.63-2.53 (m, 1H), 2.48-2.39 (m, 1H), 1.37 (d, J=11.4 Hz, 9H).

### Int. 411: Preparation of 7-bromo-5-chloro-1-methyl-1H-indole

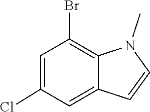

### Step 1: Int. 411

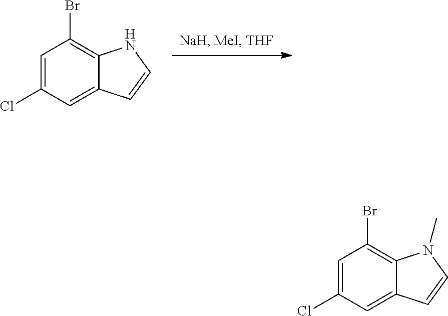

7-bromo-5-chloro-1H-indole (500 mg, 1 Eq, 2.17 mmol) and THF (10 mL) were cooled to 0° C. and 60% NaH in mineral oil (174 mg, 2 Eq, 4.34 mmol) was added in portions. MeI (924 mg, 407 μL, 3 Eq, 6.51 mmol) was added after 15 min. The reaction was warmed to RT and continued until the complete consumption of starting material (monitored by LCMS). The reaction was carefully quenched with water and was partitioned between water (20 mL) and EtOAc (30 mL). the organic layer was washed with brine, dried with MgSO₄, concentrated, and purified by flash column chromatography (SiO₂, 0 to 30% EtOAc in Hexanes). 7-bromo-5-chloro-1-methyl-1H-indole (394 mg, 1.61 mmol, 74.3%) was isolated as a white solid. LCMS (ESI+): m/z 245.5 (M+H⁺).

### Int. 412: Preparation 4-bromo-6-chloro-1-methyl-1H-indole

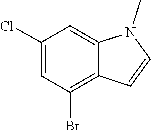

Int. 412 was synthesized using the procedure outlined in Int. 411 using 4-bromo-6-chloro-1-methyl-1H-indole as starting material. LCMS (ESI+): m/z 245.5 (M+H⁺).

### Int. 414: Preparation of 2-((1-methylpiperidin-4-yl)oxy)-5-(4,4,5,5-tetramethyl-1,3,2-dioxaborolan-2-yl)pyridine

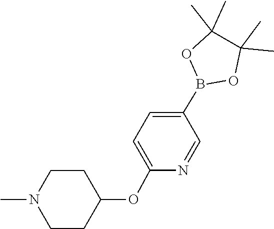

### Step 1: 5-bromo-2-((1-methylpiperidin-4-yl)oxy)pyridine

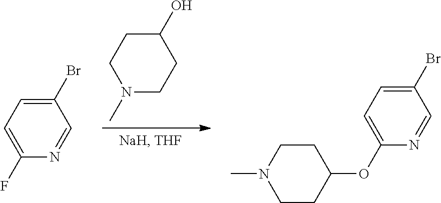

To a solution of 1-methylpiperidin-4-ol (3.31 g, 28.7 mmol, 3.36 mL, 1.1 eq) in THF (50 mL) was added 60% NaH (2.09 g, 52.2 mmol, 2.0 eq) at 0° C. The reaction was stirred at this temperature for 30 min, then 5-bromo-2-fluoropyridine (4.6 g, 26.1 mmol, 2.69 mL, 1.0 eq) was added and the reaction vessel was warmed to 25° C. and stirred for 2.5 h The reaction mixture was quenched with saturated NH₄Cl (50 mL), and the aqueous phase was extracted with EtOAc (80 mL×3). The combined organic layers were dried over Na₂SO₄, filtered, and concentrated to give 5-bromo-2-((1-methylpiperidin-4-yl)oxy)pyridine (7.8 g, crude) as a yellow oil. The product was used without further purification. LCMS: (ESI+): m/z 271.0 (M+H⁺).

### Step Int. 414

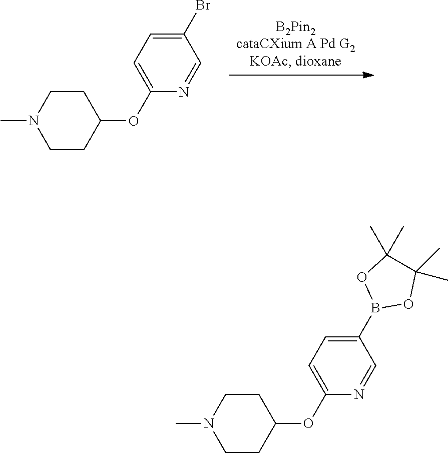

A mixture of 5-bromo-2-((1-methylpiperidin-4-yl)oxy)pyridine (7.4 g, 27.3 mmol, 1.0 eq), B₂Pin₂ (8.32 g, 32.7 mmol, 1.2 eq), KOAc (6.70 g, 68.23 mmol, 2.5 eq) and [2-(2-aminophenyl)phenyl]-chloro-palladium; bis(1-adamantyl)-butyl-phosphane (1.82 g, 2.73 mmol, 0.1 eq) in dioxane (74 mL) was degassed with N₂ for 15 min. The mixture was stirred at 120° C. for 12 hrs under N₂ atmosphere. After cooling to ambient temperature, the reaction mixture was concentrated and the resultant mixture was dissolved in EtOAc (100 mL), filtered, and concentrated under reduced pressure. The crude residue was purified by flash silica gel chromatography (SiO₂, 0-2% Methanol/EtOAc) to give Int. 414 (1.8 g, 5.32 mmol, 19.49%) as a yellow solid. LCMS: (ESI+): m/z 319.1 (M+H⁺). ¹H NMR (400 MHz, DMSO-d₆): δ 8.36 (s, 1H), 7.85 (dd, J=8.4, 1.6 Hz, 1H), 6.75 (d, J=8.4 Hz, 1H), 5.03 (dt, J=8.4, 4.0 Hz, 1H), 2.58-2.64 (m, 2H), 2.17 (s, 3H), 2.13 (s, 2H), 1.94 (d, J=9.2 Hz, 2H), 1.63-1.69 (m, 2H), 1.28 (s, 12H).

### Int. 415: Preparation of (2S,4R)-1-(1-chlorocyclopropane-1-carbonyl)-4-fluoropyrrolidine-2-carboxylic acid

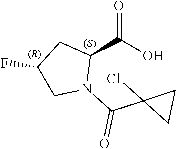

### Step 1: (cyclopropylsulfinyl)benzene

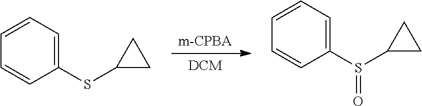

To a stirred mixture of cyclopropyl(phenyl)sulfane (4.8 g, 31.95 mmol, 4.59 mL, 1 eq) in DCM (50 mL) was added m-CPBA (7.13 g, 35.14 mmol, 1.1 eq, 85 wt. %) at 0° C. under N₂ atmosphere. The resulting mixture was stirred at 0° C. for 0.5 hr under nitrogen atmosphere until the complete consumption of starting material (monitored by LCMS). The reaction mixture was quenched with a Na₂SO₃ solution (10%, 50 mL), extracted with DCM (2×100 mL). The combined organics were washed with NaOH (5%, 50 mL) and brine (50 mL), dried over Na₂SO₄, filtered and concentrated under reduced pressure to give (cyclopropylsulfinyl)benzene (5.2 g, crude) as a light-yellow oil. LCMS (ESI+): m/z 167.2 (M+H⁺). ¹H NMR (400 MHz, CDCl₃-d): δ7.61-7.59 (m, 2H), 7.45-7.44 (m, 3H), 2.22-2.18 (m, 1H), 1.18-0.99 (m, 1H), 0.97-0.87 (m, 3H)

### Step 2: ((1-chlorocyclopropyl)sulfinyl)benzene

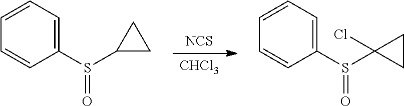

To a stirred mixture of (cyclopropylsulfinyl)benzene (5.2 g, 31.28 mmol, 1 eq) in CHCl₃ (50 mL) was added 1-chloropyrrolidine-2,5-dione (5.01 g, 37.54 mmol, 1.2 eq) at 20° C. under nitrogen atmosphere until the complete consumption of starting material (monitored by LCMS). The residue was diluted with H₂O (50 mL) and extracted with EtOAc (3×50 mL). The combined organic layers were washed with brine (50 mL), dried over anhydrous Na₂SO₄, filtered, and concentrated under reduced pressure. The resulting residue was purified by column chromatography (SiO₂, 0-50% EtOAc in Pet. ether). ((1-chlorocyclopropyl)sulfinyl)benzene (5.4 g, 23.09 mmol, 73.8%) was isolated as a clear oil after concentration. LCMS (ESI+): m/z 201.1 (M+H⁺). ¹H NMR (400 MHz, CDCl₃-d) δ7.71-7.59 (m, 2H), 7.54-7.43 (m, 3H), 1.66-1.55 (m, 2H), 1.34-1.25 (m, 1H), 1.23-1.13 (m, 1H)

### Step 3: 1-chlorocyclopropane-1-carboxylic acid

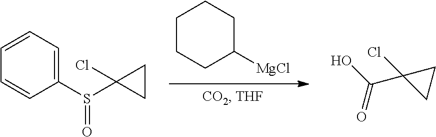

To a stirred mixture of ((1-chlorocyclopropyl)sulfinyl)benzene in THF (60 mL) was added cyclohexylmagnesium chloride (1.3 M, 16.87 mL, 1 eq) and purged with Ar −78° C. After 60 min, gaseous carbon dioxide was bubbled into the reaction for 2 h Upon completion (monitored by TLC), the residue was diluted with a saturated solution of NH₄Cl (100 mL), and the reaction mixture was concentrated under reduced pressure to remove THF. Then, the pH was adjusted with saturated NaHCO₃ (50 mL) and extracted with MTBE (3×100 mL). Next, the aqueous phase was acidified with 1N HCl (200 mL). When the pH was ˜2-3 the aqueous layer was extracted with EtOAc (3×100 mL). The combined organic layers were dried over anhydrous Na₂SO₄, filtered, and concentrated under reduced pressure to give 1-chlorocyclopropane-1-carboxylic acid (1.2 g, crude) as a white solid. The product was used with no further purification. ¹H NMR (400 MHz, CDCl₃-d): δ1.78-1.73 (m, 2H), 1.51-1.45 (m, 2H), 7.99 (s, 1H).

### Step 4: benzyl (2S,4R)-1-(1-chlorocyclopropane-1-carbonyl)-4-fluoropyrrolidine-2-carboxylate

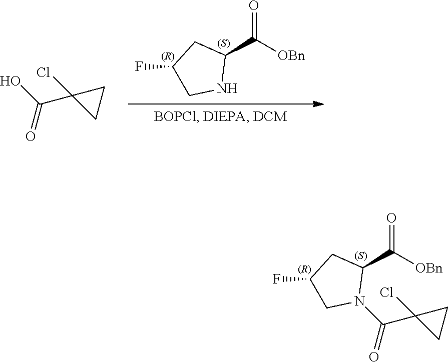

To a stirred mixture of 1-chlorocyclopropane-1-carboxylic acid (1.2 g, 9.96 mmol, 1 eq) in DCM (30 mL) was added DIPEA (1.29 g, 9.96 mmol, 1.73 mL, 1 eq) and the reaction was stirred for 30 min at ambient temperature. Then, BOPCl (2.79 g, 10.95 mmol, 1.1 eq), benzyl (2S,4R)-4-fluoropyrrolidine-2-carboxylate (2.84 g, 10.95 mmol, 1.1 eq, HCl), DIPEA (2.57 g, 19.91 mmol, 3.47 mL, 2 eq) were added sequentially. The resulting mixture was stirred at ambient temperature until the consumption of starting material was observed (monitored by LCMS), the reaction was diluted with H₂O (30 mL) and extracted with DCM (3×30 mL). The combined organic layers were washed with brine (50 mL), dried over anhydrous Na₂SO₄, then filtered and concentrated. The residue was purified by column chromatography (SiO₂, 0-50% EtOAc in pet. ether). The resulting product was further purified by trituration with petroleum ether (15 mL) to give benzyl (2S,4R)-1-(1-chlorocyclopropane-1-carbonyl)-4-fluoropyrrolidine-2-carboxylate (2.1 g, 6.45 mmol, 64.75%) as a white solid. LCMS (ESI+): m/z 326.1 (M+H⁺). ¹H NMR (400 MHz, CDCl₃-d): δ7.39-7.31 (m, 5H), 5.39-5.10 (m, 3H), 4.70 (t, J=8.6 Hz, 1H), 4.41-4.21 (m, 1H), 4.09-3.85 (m, 1H), 2.75-2.51 (m, 1H), 2.25-1.96 (m, 1H), 1.70-1.49 (m, 2H), 1.39-1.26 (m, 2H)

### Step 3: Int. 415

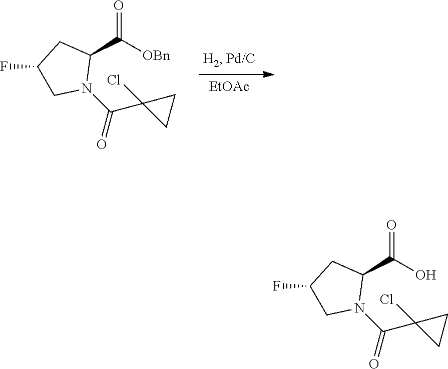 [SMILES: "EtOAc" ; O=C(OCc1ccccc1)[C@@H]1C[C@@H](F)CN1C(=O)C1(Cl)CC1 ; "H2, Pd/C" ; O=C(O)[C@@H]1C[C@@H](F)CN1C(=O)C1(Cl)CC1]

To a stirred mixture of 10% Pd/C (620.69 mg, 583.25 μmol, 0.1 eq) in EtOAc (50 mL) was added benzyl (2S,4R)-1-(1-chlorocyclopropane-1-carbonyl)-4-fluoropyrrolidine-2-carboxylate (1.9 g, 5.83 mmol, 1 eq) at 20° C. under N₂ atmosphere. The atmosphere was exchanged with H₂, and the resulting mixture was stirred under H₂ (50 Psi) at 20° C. until the complete consumption of starting material (monitored by LCMS). The reaction mixture was filtered over celite, and the filter cake rinsed with EtOAc (150 mL). The filtrate was concentrated under reduced pressure to give Int. 415 (1.25 g, 5.27 mmol, 90.39%) as a pale yellow solid. LCMS (ESI+): m/z 236.0 (M+H⁺). ¹H NMR (400 MHz, CDCl₃-d): δ8.84 (s, 1H), 5.40-5.27 (d, J=52 Hz 1H), 4.69 (t, J=8.6 Hz, 1H), 4.37 (ddd, J=1.9, 13.1, 19.6 Hz, 1H), 4.04-3.82 (m, 1H), 2.74-2.53 (m, 1H), 2.48-2.19 (m, 1H), 1.70-1.55 (m, 1H), 1.39-1.18 (m, 3H)

### Int. 416 and 417: Preparation of (2S,4R)-1-((R)-2,2-difluoro-1-methylcyclopropane-1-carbonyl)-4-fluoropyrrolidine-2-carboxylic acid and (2S,4R)-1-((S)-2,2-difluoro-1-methylcyclopropane-1-carbonyl)-4-fluoropyrrolidine-2-carboxylic acid

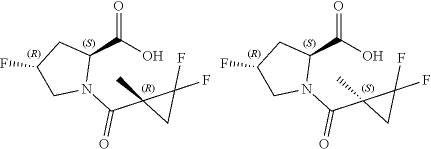

### Step 1: benzyl (2S,4R)-1-(2,2-difluoro-1-methylcyclopropane-1-carbonyl)-4-fluoropyrrolidine-2-carboxylate

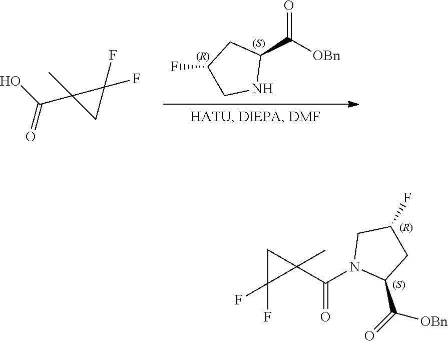

To a solution of 2,2-difluoro-1-methylcyclopropane-1-carboxylic acid (3 g, 22.04 mmol, 1 eq) in DMF (80 mL) was added DIEPA (8.55 g, 66.13 mmol, 11.52 mL, 3 eq), benzyl (2S,4R)-4-fluoropyrrolidine-2-carboxylate (6.30 g, 24.25 mmol, 1.1 eq, HCl) and HATU (16.76 g, 44.09 mmol, 2 eq) sequentially at 0° C. under a N₂ atmosphere. The mixture was stirred at 25° C. for 12 h. Water (30 mL) was added, and the aqueous phase was extracted with EtOAc (100 mL×3). The combined organic layers were dried with anhydrous Na₂SO₄ and concentrated. The resulting residue was purified by flash chromatography (SiO₂, 0-30% EtOAc in Pet. ether). This resulted in benzyl (2S,4R)-1-(2,2-difluoro-1-methylcyclopropane-1-carbonyl)-4-fluoropyrrolidine-2-carboxylate (7 g, 20.51 mmol, 93.04%) as a yellow oil. LCMS (ESI+): m/z 342.3 (M+H⁺). ¹H NMR (400 MHz, CDCl₃-d): δ7.40-7.29 (m, 5H), 5.37-5.09 (m, 3H), 4.78-4.64 (m, 1H), 4.06-3.73 (m, 2H), 2.73-2.56 (m, 1H), 2.22-2.05 (m, 1H), 2.02-1.77 (m, 1H), 1.52-1.41 (m, 3H), 1.47-1.45 (m, 1H).

### Step 2: (2S,4R)-1-(2,2-difluoro-1-methylcyclopropane-1-carbonyl)-4-fluoropyrrolidine-2-carboxylic acid

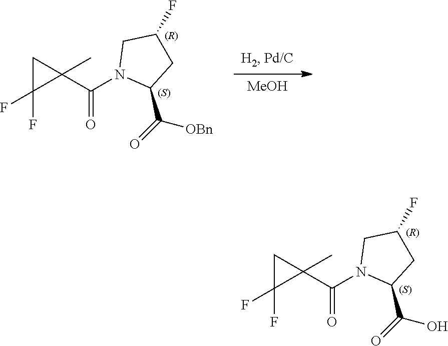

To a solution of benzyl (2S,4R)-1-(2,2-difluoro-1-methylcyclopropane-1-carbonyl)-4-fluoropyrrolidine-2-carboxylate (3 g, 8.79 mmol, 1 eq) in MeOH (50 mL) was added Pd/C (1.8 g, 1.69 mmol, 10% wt. %, 0.19 eq). The mixture was stirred under H₂ (50 psi) for 2 h The reaction solution was filtered over celite and concentrated under reduced pressure. (2S,4R)-1-(2,2-difluoro-1-methylcyclopropane-1-carbonyl)-4-fluoropyrrolidine-2-carboxylic acid (2.2 g, 8.50 mmol, 96.65%) was obtained as a white solid. ¹H NMR (400 MHz, CDCl₃-d): δ6.53 (s, 1H), 5.38-5.25 (d, J=52 Hz, 1H), 4.69 (q, J=8.8 Hz, 1H), 4.11-3.92 (m, 1H), 3.90-3.69 (m, 1H), 2.76-2.57 (m, 1H), 2.54-2.19 (m, 1H), 2.07-1.80 (m, 1H), 1.49 (d, J=1.1 Hz, 3H), 1.39-1.24 (m, 1H).

### Step 3: Int. 416, 417

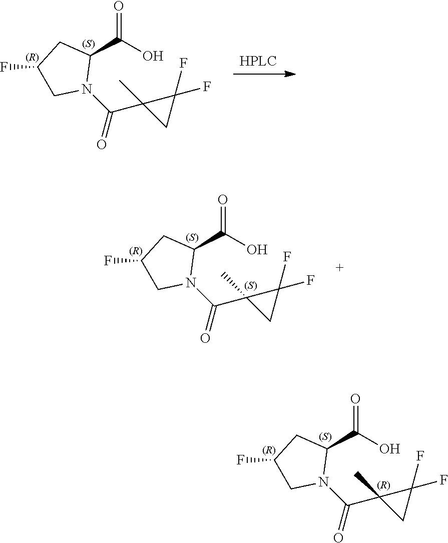

The (2S,4R)-1-(2,2-difluoro-1-methylcyclopropane-1-carbonyl)-4-fluoropyrrolidine-2-carboxylic acid was purified by prep-HPLC (Agela DuraShell C18 250×70 mm×10 um; mobile phase: [H₂O (10 mM NH₄HCO₃)/MeCN]; gradient: 0%-10% B over 17.0 min to afford Int. 416 and Int. 417.

Int. 417 (1.5 g, 5.97 mmol, 50.95%) as a white solid. LCMS (ESI+): m/z 250.1 (M−H⁺) ¹H NMR (400 MHz, DMSO-d₆): δ 12.69 (br s, 1H), 5.52-5.31 (d, J=52 Hz, 1H), 4.33 (t, J=8.4 Hz, 1H), 4.02-3.88 (m, 1H), 3.78-3.60 (m, 1H), 2.64-2.53 (m, 1H), 2.25-2.04 (m, 1H), 1.70-1.57 (m, 2H), 1.37 (s, 3H).

Int. 416 (1.3 g, 5.18 mmol, 44.16%) as a white solid. LCMS (ESI+): m/z 250.1 (M−H⁺), ¹H NMR (400 MHz, DMSO-d₆): δ 5.44-5.31 (d, J=52 Hz, 1H), 4.34 (t, J=8.4 Hz, 1H), 3.92-3.69 (m, 2H), 2.66-2.53 (m, 1H), 2.21-2.01 (m, 1H), 1.90-1.79 (m, 1H), 1.63-1.52 (m, 1H), 1.40 (s, 3H)

### Int. 419: Preparation of 2-(3-fluoro-3-methylazetidin-1-yl)-5-(4,4,5,5-tetramethyl-1,3,2-dioxaborolan-2-yl)pyridine

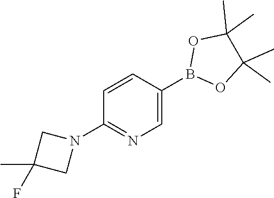

### Step 1: Int. 419

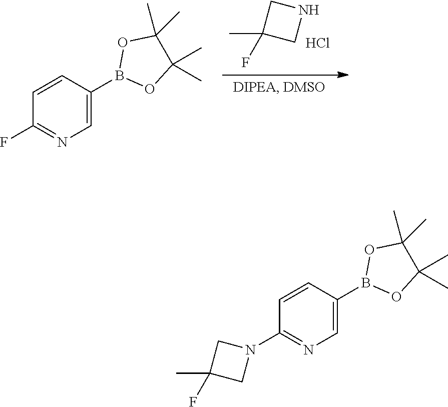

A mixture of 2-fluoro-5-(4,4,5,5-tetramethyl-1,3,2-dioxaborolan-2-yl)pyridine (3 g, 13.45 mmol, 1 eq), 3-fluoro-3-methylazetidine hydrochloride (1.69 g, 13.45 mmol, 1 eq), DIPEA (5.21 g, 40.35 mmol, 7.03 mL, 3 eq) in DMSO (50 mL), was heated to 100° C. under an atmosphere of nitrogen. The reaction continued until the complete consumption of starting material (monitored by LCMS). The reaction mixture was cooled to room temperature and diluted with water (50 mL), extracted with EtOAc (2×50 mL). The combined organics were washed with brine (50 mL), dried over Na₂SO₄, filtered, and concentrated. A second batch of this reaction (using the above conditions and scale) was added at this point. The combined residues were triturated with MTBE (40 mL) and filtered. The filter cake was dried to give Int. 419 (2.29 g, 7.84 mmol, 29.14%) as a yellow solid. LCMS (ESI+): m/z 211.1 (M−80+H⁺). ¹H NMR (400 MHz, MeOD-d₄): δ8.33 (s, 1H), 7.82 (dd, J=1.7, 8.4 Hz, 1H), 6.43 (d, J=8.4 Hz, 1H), 4.24-4.00 (m, 4H), 1.76-1.60 (d, J=21 Hz, 3H), 1.33 (s, 12H)

### Int. 421: Preparation of 2-methyl-7-(4,4,5,5-tetramethyl-1,3,2-dioxaborolan-2-yl)-1,2,3,4-tetrahydroisoquinoline

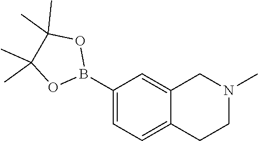

### Step 1: 7-bromo-2-methyl-1,2,3,4-tetrahydroisoquinoline

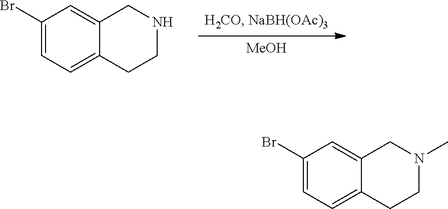

To a solution of 7-bromo-1,2,3,4-tetrahydroisoquinoline (2.5 g, 11.79 mmol, 1 eq) in MeOH (50 mL) was added H₂CO (37% in H₂O, 4.78 g, 58.94 mmol, 4.39 mL, 5 eq) and the suspension was stirred at 25° C. for 1 h. Then, the reaction was cooled to 0° C. and NaBH(OAc)₃ (3.00 g, 14.15 mmol, 1.2 eq) was added. The reaction was warmed to ambient temperature and continued until the complete consumption of starting material (monitored by LCMS). The reaction mixture was concentrated under reduced pressure to remove MeOH, and diluted with water (20 mL), extracted with MTBE (25 mL). The aqueous phase was then adjusted to pH 7 with saturated Na₂HCO₃, extracted with EtOAc (3×20 mL), the combined organic layers were washed with brine (50 mL), dried over Na₂SO₄, filtered, and concentrated under reduced pressure to give 7-bromo-2-methyl-1,2,3,4-tetrahydroisoquinoline (4.5 g, 18.51 mmol, 78.51%) as a colorless liquid without purification. The product was used with no further purification. LCMS (ESI+): m/z 226.2 (M+H⁺). ¹H NMR (400 MHz, CDCl₃-d): δ7.30-7.32 (m, 1H), 7.20 (s, 1H), 7.01 (d, J=8.2 Hz, 1H), 3.58 (s, 2H), 2.93-2.87 (t, 2H), 2.76-2.69 (t, 2H), 2.49 (s, 3H)

### Step 2: Int. 421

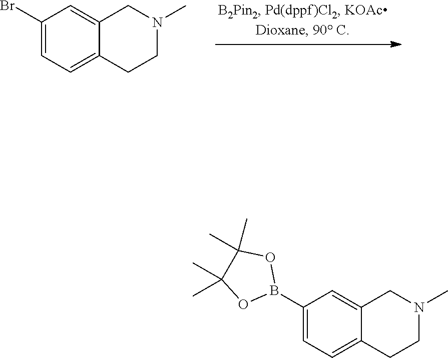

To a solution of 7-bromo-2-methyl-1,2,3,4-tetrahydroisoquinoline (4.5 g, 19.90 mmol, 1 eq) in dioxane (50 mL) was added B₂Pin₂ (7.58 g, 29.85 mmol, 1.5 eq) and KOAc (4.88 g, 49.75 mmol, 2.5 eq) under nitrogen atmosphere. The suspension was degassed with N₂ (15 min) and Pd(dppf)Cl₂·CH₂Cl₂ (1.63 g, 1.99 mmol, 0.1 eq) was added. The reaction mixture was heated to 90° C. until the complete consumption of starting material (monitored by LCMS). The reaction mixture was cooled to ambient temperature and concentrated. The crude residue was diluted with sat. NaHCO₃ (30 mL), extracted with EtOAc (3×30 mL). The combined organic layers were washed with brine (50 mL), dried over Na₂SO₄, filtered, and concentrated under reduced pressure. The resulting crude product was purified by flash chromatography (SiO₂, 0-6% MeOH in DCM) to give Int. 421 (2.1 g, 7.10 mmol, 35.69%) was obtained as a black solid. LCMS (ESI+): m/z 274.1 (M+H⁺). ¹H NMR (400 MHz, CDCl₃-d): δ7.57 (d, J=7.6 Hz, 1H), 7.48 (s, 1H), 7.12 (d, J=7.5 Hz, 1H), 3.60 (s, 2H), 2.94 (t, J=5.8 Hz, 2H), 2.73-2.66 (m, 2H), 2.45 (d, J=1.1 Hz, 3H), 1.34 (s, 12H).

### Int. 424: Preparation of 2-methyl-6-(4-(4,4,5,5-tetramethyl-1,3,2-dioxaborolan-2-yl)phenyl)-2,6-diazaspiro[3.4]octane

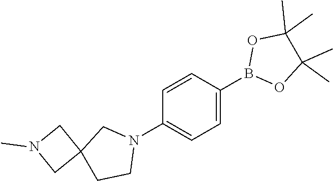

### Step 1: tert-butyl 6-(4-chlorophenyl)-2,6-diazaspiro[3.4]octane-2-carboxylate

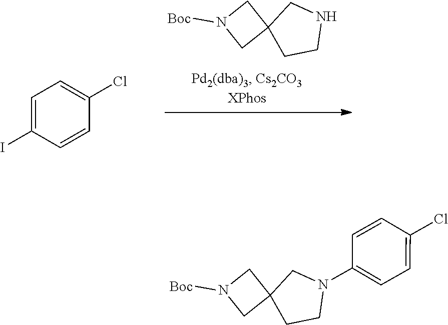 [SMILES: CC(C)(C)OC(=O)N1CC2(CCNC2)C1 ; Clc1ccc(I)cc1 ; CC(C)(C)OC(=O)N1CC2(CCN(c3ccc(Cl)cc3)C2)C1]

To a stirred mixture of 4-chloroiodobenzene (5 g, 20.969 mmol, 1 eq) and tert-butyl 2,6-diazaspiro[3.4]octane-2-carboxylate (6.68 g, 31.454 mmol, 1.5 eq) in dioxane (50 mL) were added Pd₂(dba)₃ (2.17 g, 2.097 mmol, 0.1 eq), XPhos (2.00 g, 4.194 mmol, 0.2 eq) and Cs₂CO₃ (20.50 g, 62.907 mmol, 3 eq) in portions at room temperature under nitrogen atmosphere. The resulting mixture was stirred for additional overnight at 80° C. The resulting mixture was extracted with EtOAc (2×100 mL). The combined organic layers were dried over anhydrous Na₂SO₄. After filtration, the filtrate was concentrated under reduced pressure. The residue was purified RP-HPLC (C18, 10-100% MeCN in water with 0.1% FA). Tert-butyl 6-(4-chlorophenyl)-2,6-diazaspiro[3.4]octane-2-carboxylate (4.6 g, 67.95%) was recovered as a brown solid. LCMS (ESI+): m/z 323.1 (M+H⁺).

### Step 2-3 6-(4-chlorophenyl)-2-methyl-2,6-diazaspiro[3.4]octane

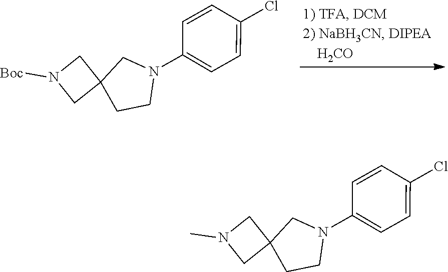 [SMILES: CC(C)(C)OC(=O)N1CC2(CCN(c3ccc(Cl)cc3)C2)C1 ; CN1CC2(CCN(c3ccc(Cl)cc3)C2)C1]

A mixture of tert-butyl 6-(4-chlorophenyl)-2,6-diazaspiro[3.4]octane-2-carboxylate (1 g, 3.098 mmol, 1 eq) and TFA (3 mL) in DCM (15 mL) was stirred overnight at room temperature. The resulting mixture was concentrated to afford resulted 6-(4-chlorophenyl)-2,6-diazaspiro[3.4]octane (530 mg, 76.82%) as a brown oil. The product was used in the next step without further purification. LCMS (ESI+): m/z 223.15 (M+H⁺).

To a stirred mixture of 6-(4-chlorophenyl)-2,6-diazaspiro[3.4]octane (700 mg, 3.143 mmol, 1 eq) and DIEA (812.44 mg, 6.29 mmol, 2 eq) in 1:1 MeOH:THF (4 mL) were added H₂CO (0.86 mL, 9.42 mmol, 3 eq, 40% in water) and NaBH₃CN (592.5 mg, 9.43 mmol, 3 eq) in portions at room temperature under an atmosphere of nitrogen. The resulting mixture was stirred for an additional 2 h at room temperature. The reaction was directly purified by RP-HPLC (C18, 10-100% MeCN in water with 0.1% FA) to afford 6-(4-chlorophenyl)-2-methyl-2,6-diazaspiro[3.4]octane (150 mg, 20.16%) an oil. LCMS (ESI+): m/z 237.15 (M+H⁺).

### Step 4: Int. 424

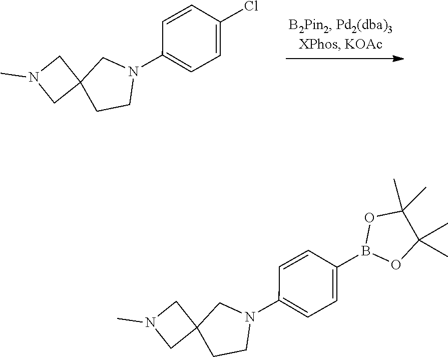

To a stirred mixture of 6-(4-chlorophenyl)-2-methyl-2,6-diazaspiro[3.4]octane (1.2 g, 5.069 mmol, 1 eq) and bis(pinacolato)diboron (1.93 g, 7.604 mmol, 1.5 eq) in dioxane (20 mL) were added Pd₂(dba)₃ (0.52 g, 0.507 mmol, 0.1 eq), XPhos (0.48 g, 1.014 mmol, 0.2 eq) and KOAc (1.49 g, 15.207 mmol, 3 eq) under nitrogen. The reaction was heated to 100° C. until consumption of starting material was observed (LCMS). The resulting mixture was extracted with EtOAc (2×100 mL). The combined organic layers were dried over anhydrous Na₂SO₄. After filtration, the filtrate was concentrated under reduced pressure. The residue was purified by Preparative TLC (CH₂Cl₂/MeOH 10:1) to afford Int. 424 (0.2993 g, 15.00%) as a yellow green solid. LCMS (ESI+): m/z 329.15 (M+H⁺). ¹H NMR (400 MHz, DMSO-d6) δ 7.46 (d, J=8.1 Hz, 1H), 7.23-7.05 (m, 1H), 6.48 (t, J=8.9 Hz, 2H), 3.35 (s, 2H), 3.28-3.09 (m, 6H), 2.27 (d, J=2.5 Hz, 3H), 1.16 (d, J=71.8 Hz, 12H).

### Int. 425: Preparation of (S)-1,2-dimethyl-4-(4-(4,4,5,5-tetramethyl-1,3,2-dioxaborolan-2-yl)phenyl)piperazine

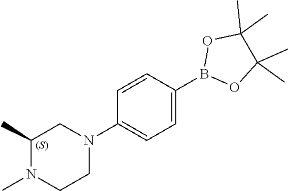

### Step 1: (S)-4-(4-bromophenyl)-1,2-dimethylpiperazine

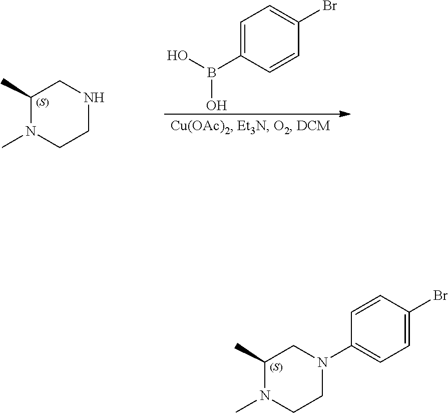

To a solution of (S)-1,2-dimethylpiperazine (3 g, 26.27 mmol, 1 eq) and (4-bromophenyl)boronic acid (7.91 g, 39.41 mmol, 1.5 eq) in CH₂Cl₂ (100 mL) was added Cu(OAc)₂ (954.39 mg, 5.25 mmol, 0.2 eq), Et₃N (10.63 g, 105.09 mmol, 14.63 mL, 4 eq), and 4 Å molecular sieves (1 g). The mixture was (C18, 10-100% MeCN in water with 0.1% FA). SiO2, 0-100% EtOAc in pet. ether (C18, 10-100% MeCN in water with 0.1% FA). SiO₂, 0-100% EtOAc in pet. ether (C18, 10-100% MeCN in water with 0.1% FA). stirred at ambient temperature under an atmosphere of oxygen (15 psi). The reaction continued until the consumption of starting materials (monitored by LCMS). The resulting suspension was filtered through a pad of silica gel and the filter cake was washed with EtOAc (20 mL×3). The combined filtrates were concentrated to dryness and the residue was purified by flash silica gel chromatography (SiO₂, 0-100% EtOAc in pet. ether). (S)-4-(4-bromophenyl)-1,2-dimethylpiperazine (3 g, 11.14 mmol, 42.42%) was obtained as a gray solid. LCMS: (ESI+): m/z 269.3 (M+H⁺). ¹H NMR (400 MHz, CDCl₃-d): δ7.33 (d, J=8.8 Hz, 2H), 6.77 (d, J=8.8 Hz, 2H), 3.51-3.36 (m, 2H), 3.01-2.87 (m, 2H), 2.62-2.43 (m, 2H), 2.42-2.29 (m, 4H), 1.17 (d, J=6.4 Hz, 3H).

### Step 2: Int. 425

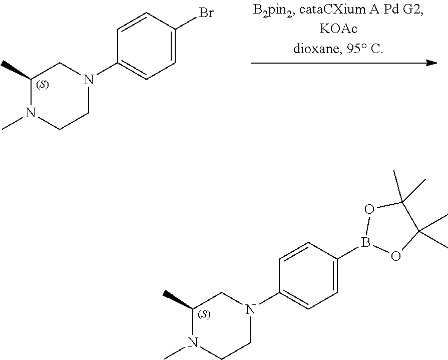

A mixture of (S)-4-(4-bromophenyl)-1,2-dimethylpiperazine (2.9 g, 10.77 mmol, 1 eq), B₂Pin₂ (3.28 g, 12.93 mmol, 1.2 eq), KOAc (2.64 g, 26.93 mmol, 2.5 eq) and [2-(2-aminophenyl)phenyl]-chloro-palladium; bis(1-adamantyl)-butyl-phosphane (720.34 mg, 1.08 mmol, 0.1 eq) in dioxane (50 mL) was degassed with N₂ for 20 min. The reaction was heated to 95° C. for 4 h. The suspension was filtered through a pad of silica gel and the filter cake was washed with EtOAc (20 mL×3). The combined filtrates were concentrated to dryness and the residue was purified by flash silica gel chromatography (SiO₂, 0-100% EtOAc in Pet. ether). Int. 425 (2 g, 6.32 mmol, 58.70%) was obtained as a gray solid. LCMS: (ESI+): m/z 317.2 (M+H⁺), ¹H NMR (400 MHz, CDCl₃-d) δ7.71 (d, J=8.4 Hz, 2H), 6.89 (d, J=8.4 Hz, 2H), 3.69-3.50 (m, 2H), 3.02-2.87 (m, 2H), 2.65-2.55 (m, 1H), 2.41 (dt, J=3.2, 11.4 Hz, 1H), 2.37-2.32 (m, 3H), 2.27 (ddd, J=3.2, 6.4, 9.7 Hz, 1H), 1.33 (s, 12H), 1.15 (d, J=6.4 Hz, 3H).

### Int. 426: Preparation of (S)—N,N-dimethyl-1-(4-(4-(4,4,5,5-tetramethyl-1,3,2-dioxaborolan-2-yl)phenyl)morpholin-2-yl)methanamine

### Step 1: (R)-(4-(4-bromophenyl)morpholin-2-yl)methanol

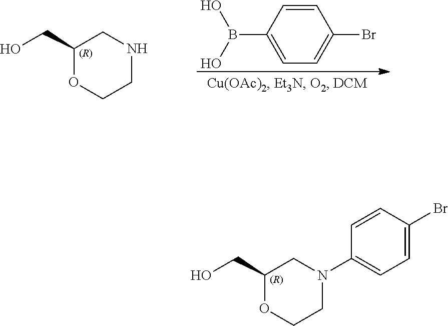

Step 1 was performed using the procedure outline in step 1 for the synthesis of Int. 425 using (R)-morpholin-2-ylmethanol as starting material. LCMS (ESI+): m/z 272.0 (M+H⁺). ¹H NMR (400 MHz, CDCl₃-d): δ7.42-7.34 (m, 2H), 6.83 (d, J=8.6 Hz, 2H), 4.12-4.03 (m, 1H), 3.95-3.63 (m, 4H), 3.46-3.34 (m, 2H), 2.86 (dt, J=3.4, 11.9 Hz, 1H), 2.69 (t, J=11.1 Hz, 1H), 1.93 (s, 0.5H), 1.51 (s, 0.5H)

### Step 2: (R)-(4-(4-(4,4,5,5-tetramethyl-1,3,2-dioxaborolan-2-yl)phenyl)morpholin-2-yl)methanol

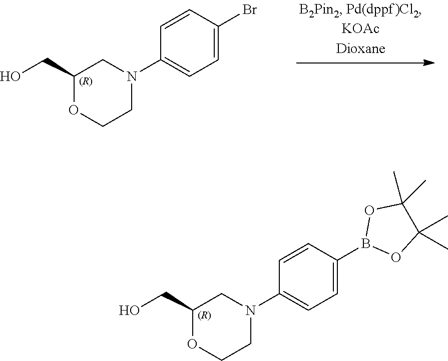

To a solution of (R)-(4-(4-bromophenyl)morpholin-2-yl)methanol (2.5 g, 9.19 mmol, 1 eq), bis(pinacolato)diboron (3.50 g, 13.78 mmol, 1.5 eq), KOAc (2.70 g, 27.56 mmol, 3 eq) in dioxane (25 mL) was degassed with N₂ for 10 min. Pd(dppf)Cl₂ (750.21 mg, 918.65 μmol, 0.1 eq) was added to the reaction. The mixture was heated to 100° C. until the complete consumption of starting material (monitored by LCMS). The reaction mixture was diluted with water (50 mL), extracted with EtOAc (2×50 mL). The combined organics were washed with brine (50 mL), dried over Na₂SO₄, filtered, and concentrated under reduced pressure. The residue was purified by flash silica gel chromatography (SiO₂, 0-40% EtOAc in pet. ether) to give (R)-(4-(4-(4,4,5,5-tetramethyl-1,3,2-dioxaborolan-2-yl)phenyl)morpholin-2-yl)methanol (1.7 g, 5.06 mmol, 55.08%) as a yellow solid. LCMS (ESI+): m/z 320.2 (M+H⁺). ¹H NMR (400 MHz, CDCl₃-d): δ7.74 (d, J=8.6 Hz, 2H), 6.94 (d, J=7.1 Hz, 2H), 4.11-4.04 (m, 1H), 3.95-3.65 (m, 4H), 3.60-3.50 (m, 2H), 2.99-2.69 (m, 2H), 2.04-1.91 (m, 1H), 1.34 (s, 12H).

### Step 3: (R)-(4-(4-(4,4,5,5-tetramethyl-1,3,2-dioxaborolan-2-yl)phenyl)morpholin-2-yl)methyl methanesulfonate

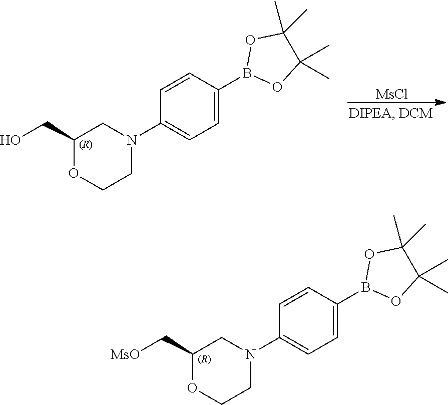

To a solution of (R)-(4-(4-(4,4,5,5-tetramethyl-1,3,2-dioxaborolan-2-yl)phenyl)morpholin-2-yl)methanol (1.6 g, 5.01 mmol, 1 eq) in dichloromethane (80 mL) was added DIPEA (3.24 g, 25.06 mmol, 4.37 mL, 5 eq) at 0° C. Then MsCl (918.70 mg, 8.02 mmol, 620.74 μL, 1.6 eq) was added to the reaction dropwise under a N₂ atmosphere at 0° C. The reaction was warmed to ambient temperature and stirred until the consumption of starting material (monitored by LCMS). The reaction mixture was cooled, diluted with water (50 mL), and extracted with EtOAc (2×50 mL). The combined organic layers were washed with brine (50 mL), dried over Na₂SO₄, filtered, and concentrated under reduced pressure to give (R)-(4-(4-(4,4,5,5-tetramethyl-1,3,2-dioxaborolan-2-yl)phenyl)morpholin-2-yl)methyl methanesulfonate (2 g, 4.53 mmol, 90.39%) as a yellow oil. LCMS (ESI+): m/z 398.2 (M+H⁺). ¹H NMR (400 MHz, CDCl₃-d): δ7.74 (d, J=8.6 Hz, 2H), 6.89 (d, J=8.6 Hz, 2H), 4.37-4.30 (m, 2H), 4.10-4.04 (m, 1H), 4.01-3.91 (m, 1H), 3.81 (dt, J=2.7, 11.5 Hz, 1H), 3.64-3.48 (m, 2H), 3.10 (s, 3H), 2.91 (dt, J=3.4, 11.9 Hz, 1H), 2.77-2.68 (m, 1H), 1.34 (s, 12H)

### Step 4: Int. 426

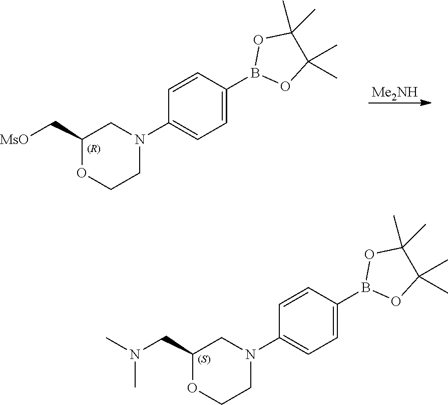

A solution of (R)-(4-(4-(4,4,5,5-tetramethyl-1,3,2-dioxaborolan-2-yl)phenyl)morpholin-2-yl)methyl methanesulfonate (2 g, 5.03 mmol, 1 eq) in dimethylamine (2 M in THF, 42.79 mL, 17 eq) was added to 100 mL Teflon tank. The reaction was stirred at 75° C. for 12 h. The reaction mixture was diluted with water (50 mL), extracted with EtOAc (2×50 mL). The combined organic layers were washed with brine (50 mL), dried over Na₂SO₄, filtered, and concentrated under reduced pressure to give a residue. The residue was purified by prep-HPLC (heptane/EtOH with 0.1% iso-propylamine) to give Int. 426 (0.85 g, 2.30 mmol, 45.68%) as a white solid. LCMS (ESI+): m/z 347.2 (M+H⁺). ¹H NMR (400 MHz, MeOD-d₄): δ7.62 (d, J=8.6 Hz, 2H), 6.93 (d, J=8.8 Hz, 2H), 4.01 (td, J=1.8, 9.8 Hz, 1H), 3.81-3.68 (m, 2H), 3.67-3.52 (m, 2H), 2.79 (dt, J=3.4, 11.9 Hz, 1H), 2.59-2.40 (m, 3H), 2.31 (s, 6H), 1.32 (s, 12H).

### Int. 427 and Int. 428: Preparation of (R)-1-methyl-3-(4-(4,4,5,5-tetramethyl-1,3,2-dioxaborolan-2-yl)phenyl)piperidine and (S)-1-methyl-3-(4-(4,4,5,5-tetramethyl-1,3,2-dioxaborolan-2-yl)phenyl)piperidine

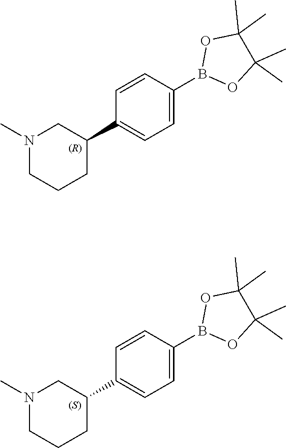

### Step 1: tert-butyl 5-(4-bromophenyl)-3,6-dihydropyridine-1 (2H)-carboxylate

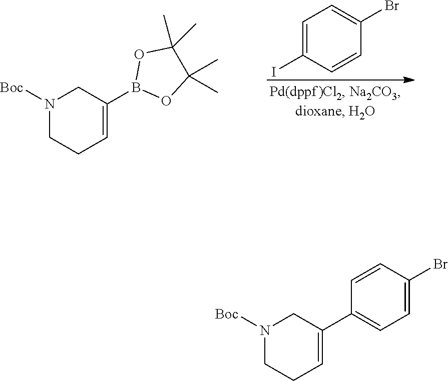 [SMILES: CC(C)(C)OC(=O)N1CCC=C(B2OC(C)(C)C(C)(C)O2)C1 ; Brc1ccc(I)cc1 ; CC(C)(C)OC(=O)N1CCC=C(c2ccc(Br)cc2)C1]

To a solution of tert-butyl 5-(4,4,5,5-tetramethyl-1,3,2-dioxaborolan-2-yl)-3,6-dihydropyridine-1 (2H)-carboxylate (10.0 g, 32.34 mmol, 1 eq) in dioxane (200 mL) and H₂O (35 mL) was added Pd(dppf)Cl₂ (236.64 mg, 323.41 μmol, 0.01 eq) and Na₂CO₃ (6.86 g, 64.68 mmol, 2 eq) and 1-bromo-4-iodo-benzene (9.15 g, 32.34 mmol, 1 eq). The mixture was stirred at 80° C. for 1 h. The reaction mixture was diluted with water (100 mL) and extracted with EtOAc (80 mL×3). The combined organic layers were dried over Na₂SO₄, filtered, and concentrated under reduced pressure to give a residue. The residue was purified by column chromatography (SiO₂, Pet. ether/EtOAc 0-15%). tert-butyl 5-(4-bromophenyl)-3,6-dihydropyridine-1 (2H)-carboxylate (14 g, 41.39 mmol, 63.99%) was obtained as a clear oil. LCMS (ESI+): m/z 338.1 (M+H⁺). ¹H NMR (400 MHz, CDCl₃-d): δ7.46 (d, J=8.4 Hz, 2H), 7.24 (d, J=8.4 Hz, 2H), 6.20 (td, J=2.0, 4.0 Hz, 1H), 4.23 (s, 2H), 3.55 (t, J=6.0 Hz, 2H), 2.31 (d, J=3.6 Hz, 2H), 1.50 (s, 9H).

### Step 2: tert-butyl 3-(4-bromophenyl)piperidine-1-carboxylate

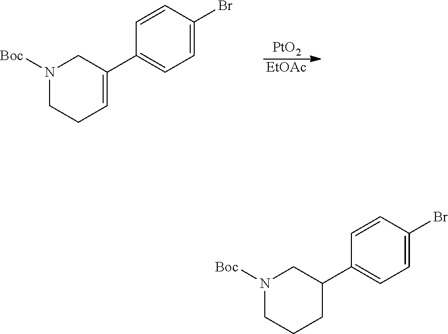

To a suspension of PtO₂ (0.5 g) of in EtOAc (80 mL) was added and tert-butyl 5-(4-bromophenyl)-3,6-dihydropyridine-1 (2H)-carboxylate (5 g, 14.78 mmol, 1 eq) and the mixture was stirred at 20° C. for 2 h under H₂ atmosphere. The reaction mixture was filtered through a pad of silica gel and the filter cake was washed with EtOAc (50 mL×3). The combined filtrates were concentrated to dryness and the residue was purified by column chromatography (SiO₂, EtOAc:PE 0-50%) to afford tert-butyl 3-(4-bromophenyl)piperidine-1-carboxylate (5 g, 14.69 mmol, 99.41%) as a white oil. LCMS (ESI+): m/z 340.0 (M+H⁺) ¹H NMR (400 MHz, CDCl₃-d): δ7.44 (d, J=8.4 Hz, 2H), 7.11 (d, J=8.4 Hz, 2H), 4.20-4.12 (m, 2H), 2.73 (d, J=11.2 Hz, 2H), 2.00 (d, J=9.2 Hz, 1H), 1.80-1.71 (m, 1H), 1.66-1.54 (m, 3H), 1.47 (s, 9H).

### Step 3: tert-butyl 3-(4-(4,4,5,5-tetramethyl-1,3,2-dioxaborolan-2-yl)phenyl)piperidine-1-carboxylate

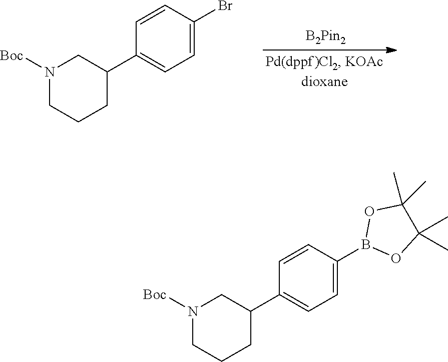 [SMILES: CC(C)(C)OC(=O)N1CCCC(c2ccc(Br)cc2)C1 ; CC(C)(C)OC(=O)N1CCCC(c2ccc(B3OC(C)(C)C(C)(C)O3)cc2)C1]

To a solution of tert-butyl 3-(4-bromophenyl)piperidine-1-carboxylate (2 g, 5.88 mmol, 1 eq) in dioxane (40 mL) was added B₂Pin₂ (2.24 g, 8.82 mmol, 1.5 eq), KOAc (1.15 g, 11.76 mmol, 2 eq) and Pd(dppf)Cl₂ (430.10 mg, 587.80 μmol, 0.1 eq). The solution was degassed with a stream of N₂ and heated to 100° C. for 1 h. Upon the consumption of the starting material (monitored by LCMS) the reaction mixture was diluted with water (30 mL) and extracted with EtOAc (30 mL×3), the combined organic layers were dried over Na₂SO₄, filtered, and concentrated. The resulting residue was purified by column chromatography (SiO₂, 0 to 10% EtOAc in pet. ether) to afford tert-butyl 3-(4-(4,4,5,5-tetramethyl-1,3,2-dioxaborolan-2-yl)phenyl)piperidine-1-carboxylate (2.2 g, 5.68 mmol, 96.63%) as a clear oil. LCMS (ESI+): m/z 388.2 (M+H⁺). ¹H NMR (400 MHz, CDCl₃-d): δ7.77 (d, J=7.6 Hz, 2H), 7.25 (d, J=8.0 Hz, 2H), 4.24-4.09 (m, 2H), 2.81-2.64 (m, 3H), 1.79-1.55 (m, 4H), 1.34 (s, 9H), 1.27 (s, 12H).

### Step 4: 3-(4-(4,4,5,5-tetramethyl-1,3,2-dioxaborolan-2-yl)phenyl)piperidine

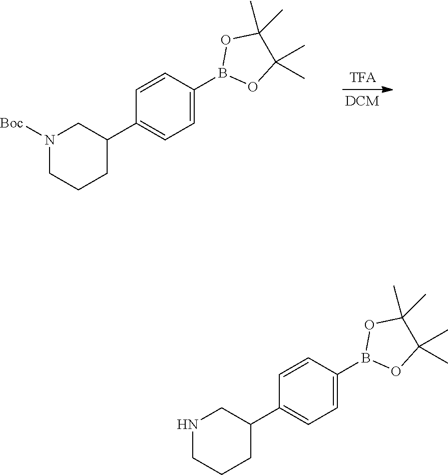 [SMILES: CC(C)(C)OC(=O)N1CCCC(c2ccc(B3OC(C)(C)C(C)(C)O3)cc2)C1 ; CC1(C)OB(c2ccc(C3CCCNC3)cc2)OC1(C)C]

To a solution of tert-butyl 3-(4-(4,4,5,5-tetramethyl-1,3,2-dioxaborolan-2-yl)phenyl)piperidine-1-carboxylate (1.05 g, 2.71 mmol, 1 eq) in DCM (20 mL) was added TFA (4.2 mL). The mixture was stirred at 25° C. for 1 h The solution was basified to pH=7 with NaHCO₃ (1 mol/L), and extracted with EtOAc (10 mL×3), dried over Na₂SO₄, filtered, and concentrated under reduced pressure to give 3-(4-(4,4,5,5-tetramethyl-1,3,2-dioxaborolan-2-yl)phenyl)piperidine (1.7 g, crude) as a white solid. The material was used directly in the next reaction. LCMS (ESI+): m/z 288.2 (M+H⁺). ¹H NMR (400 MHz, CDCl₃-d): δ7.78 (d, J=8.0 Hz, 2H), 7.20 (d, J=8.0 Hz, 2H), 3.43 (d, J=12.4 Hz, 2H), 3.16-3.04 (m, 1H), 2.96-2.82 (m, 2H), 2.14-1.95 (m, 3H), 1.76-1.62 (m, 1H), 1.34 (s, 12H).

### Step 5: 1-methyl-3-(4-(4,4,5,5-tetramethyl-1,3,2-dioxaborolan-2-yl)phenyl)piperidine

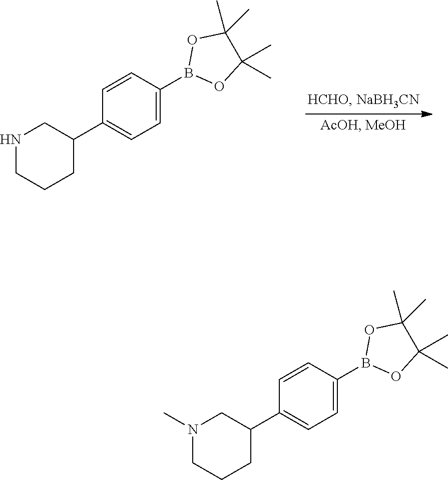

To a solution of 3-(4-(4,4,5,5-tetramethyl-1,3,2-dioxaborolan-2-yl)phenyl)piperidine (1 g, 3.48 mmol, 1 eq) in MeOH (10 mL) was added a 37% formaldehyde solution (1.41 g, 17.41 mmol, 1.30 mL, 5 eq) and AcOH (20.91 mg, 348.18 mol, 19.93 μL, 0.1 eq) at 0° C. The reaction was stirred for 30 minutes, then, NaBH₃CN (656.42 mg, 10.45 mmol, 3 eq) was added. The reaction continued for an additional 30 min. The reaction mixture was then diluted with water (20 mL) and extracted with EtOAc (15 mL×3), the combined organic layers were dried over Na₂SO₄, filtered, and concentrated. The residue was triturated with MTBE (10 mL) to give 1-methyl-3-(4-(4,4,5,5-tetramethyl-1,3,2-dioxaborolan-2-yl)phenyl)piperidine (0.2 g, 663 mol, 19.0%) as a white solid. LCMS (ESI+): m/z 302.4 (M+H⁺). ¹H NMR (400 MHz, CDCl₃-d): δ7.79 (d, J=8.0 Hz, 2H), 7.25 (d, J=8.0 Hz, 2H), 3.56-3.39 (m, 2H), 3.30-3.16 (m, 1H), 2.89 (t, J=12.4 Hz, 2H), 2.84 (s, 3H), 2.20-2.02 (m, 3H), 1.78-1.64 (m, 1H), 1.33 (s, 12H).

### Step 6: Int. 427, Int. 428

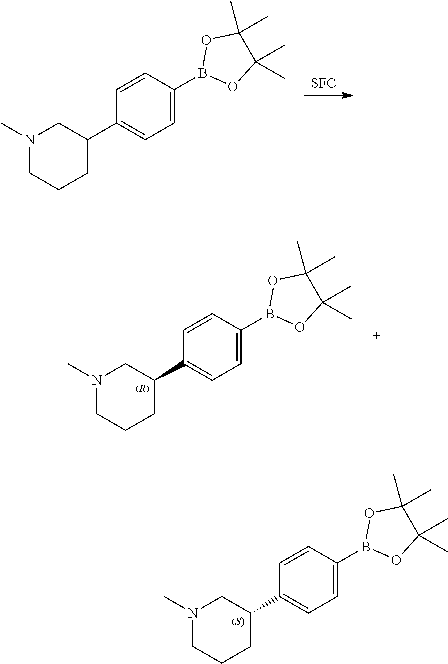

The 1-methyl-3-(4-(4,4,5,5-tetramethyl-1,3,2-dioxaborolan-2-yl)phenyl)piperidine was further separated by SFC (condition: column: Daicel Chiralcel OZ 250×50 mm; 10 m; mobile phase: [CO2-MeOH (0.1% IPAm)]; B %: 25%, isocratic elution) to afford (R)-stereochemistry Int. 428 (0.5 g, 1.56 mmol, 23.50%), LCMS (ESI+): m/z 302.4 (M+H⁺) ¹H NMR (400 MHz, CDCl₃-d): 67.76 (d, J=7.6 Hz, 2H), 7.25 (d, J=7.6 Hz, 2H), 3.01-2.81 (m, 3H), 2.31 (s, 3H), 2.02-1.89 (m, 3H), 1.83-1.68 (m, 2H), 1.43 (dd, J=4.4, 12.4 Hz, 1H), 1.34 (s, 12H), and (S)-stereochemistry Int. 427 (0.46 g, 1.39 mmol, 20.93%) as white solid. LCMS (ESI+): m/z 302.4 (M+H⁺) ¹H NMR (400 MHz, CDCl₃-d): δ7.77 (d, J=7.6 Hz, 2H), 7.24 (d, J=7.6 Hz, 2H), 3.15 (d, J=11.6 Hz, 2H), 3.03 (tt, J=3.4, 12.0 Hz, 1H), 2.49 (s, 3H), 2.32-2.20 (m, 2H), 2.02-1.87 (m, 3H), 1.57-1.48 (m, 1H), 1.34 (s, 12H).

### Int. 429: Preparation of 1-(2-fluoro-4-(4,4,5,5-tetramethyl-1,3,2-dioxaborolan-2-yl)phenyl)-4-isopropylpiperazine

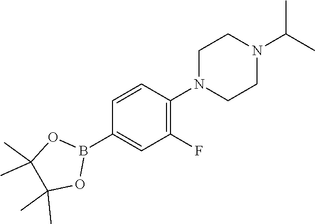 [SMILES: CC(C)N1CCN(c2ccc(B3OC(C)(C)C(C)(C)O3)cc2F)CC1]

### Step 1: 1-(4-bromo-2-fluorophenyl)-4-isopropylpiperazine

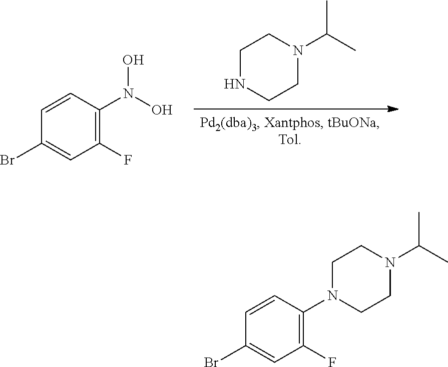

A mixture of (4-bromo-2-fluorophenyl)boronic acid (4 g, 13.29 mmol, 1 eq), 1-isopropylpiperazine (1.70 g, 13.29 mmol, 1.90 mL, 1 eq), Pd₂(dba)₃ (304.33 mg, 332.34 mol, 0.025 eq), Xantphos (769.20 mg, 1.33 mmol, 0.1 eq) and NaOt-Bu (2.56 g, 26.59 mmol, 2 eq) in Tol. (80 mL) was degassed and purged with N₂ (20 min). The mixture was stirred at 80° C. for 1 h. The reaction mixture was concentrated to give a residue and the residue was purified by flash chromatography (SiO₂, 30-35% EtOAc in pet. ether) to give 1-(4-bromo-2-fluorophenyl)-4-isopropylpiperazine (2.8 g, 7.41 mmol, 55.74%) as a brown oil. LCMS (ESI+): m/z 301.0 (M+H⁺), ¹H NMR (CDCl₃-d, 400 MHz): δ 7.15-7.22 (m, 2H), 6.82 (t, J=8.8 Hz, 1H), 3.07-3.12 (m, 4H), 2.73-2.78 (m, 1H), 2.69-2.72 (m, 4H), 1.10 (d, J=6.4 Hz, 6H).

### Step 2: Int. 429

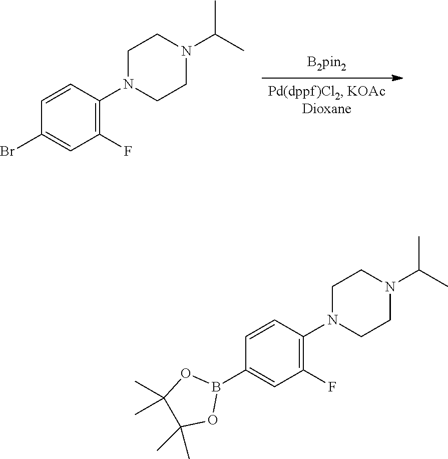 [SMILES: CC(C)N1CCN(c2ccc(Br)cc2F)CC1 ; "B2pin2" ; "Pd(dppf)Cl2, KOAcDioxane" ; CC(C)N1CCN(c2ccc(B3OC(C)(C)C(C)(C)O3)cc2F)CC1]

A mixture of 1-(4-bromo-2-fluorophenyl)-4-isopropylpiperazine (2.7 g, 8.96 mmol, 1 eq), 4,4,5,5-tetramethyl-2-(4,4,5,5-tetramethyl-1,3,2-dioxaborolan-2-yl)-1,3,2-dioxaborolane (3.41 g, 13.45 mmol, 1.5 eq), KOAc (2.20 g, 22.41 mmol, 2.5 eq), Pd(dppf)Cl₂ (327.96 mg, 448.21 μmol, 0.05 eq) in dioxane (50 mL) was degassed with N₂ for 20 min. The mixture was heated to 100° C. for 5 h. The reaction mixture was concentrated and the resulting residue was purified by flash chromatography (SiO₂, 0-5% MeOH in EtOAc) to afford Int. 429. The product further purified by salt formation with HCl in EtOAc. Int. 429 (2.3 g, 5.86 mmol, 65.32%, HCl) as a white solid. LCMS: (ESI+): m/z 349.2 (M+H⁺), ¹H NMR (400 MHz, DMSO-d₆): δ 7.44 (dd, J=7.6, 1.2 Hz, 1H), 7.31 (dd, J=13.6, 1.2 Hz, 1H), 7.09 (t, J=8.4 Hz, 1H), 3.58 (br d, J=12.8 Hz, 2H), 3.47 (d, J=11.2 Hz, 3H), 3.32 (t, J=11.6 Hz, 2H), 3.10-3.23 (m, 2H), 1.32 (d, J=6.4 Hz, 6H), 1.27 (s, 12H).

### Int. 430: Preparation of ((R)-2-methyl-4-(4-(4,4,5,5-tetramethyl-1,3,2-dioxaborolan-2-yl)phenyl)morpholine

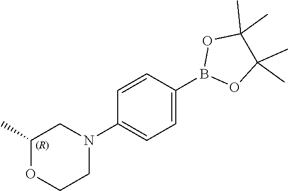

### Step 1: (R)-4-(4-bromophenyl)-2-methylmorpholine

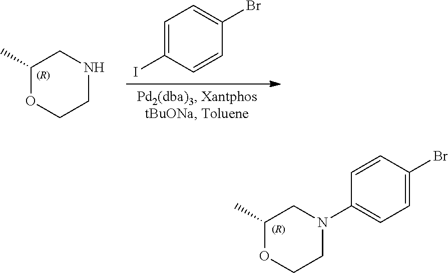

A mixture of (R)-2-methylmorpholine (3 g, 29.66 mmol, 1 eq), 1-bromo-4-iodo-benzene (10.91 g, 38.56 mmol, 1.3 eq), NaOtBu (8.55 g, 88.98 mmol, 3 eq), Xantphos (2.15 g, 3.71 mmol, 0.125 eq) and Pd₂(dba)₃ (1.36 g, 1.48 mmol, 0.05 eq) in toluene (50 mL) was degassed with N₂ for 10 min, then heated to 85° C. for 1 h. After cooling to room temperature, the reaction mixture was concentrated. The resulting residue was diluted with water (50 mL) and extracted with EtOAc (80 mL×3). The combined organic layers were dried over Na₂SO₄, filtered, and concentrated. The residue was purified by flash chromatography (SiO₂, 0-5% EtOAc in Pet. ether) to give (R)-4-(4-bromophenyl)-2-methylmorpholine (7 g, 23.27 mmol, 78.44%) as a yellow solid. LCMS: (ESI+): m/z 256.0 (M+H⁺), ¹H NMR (400 MHz, DMSO-d₆): δ 7.35 (d, J=9.2 Hz, 2H), 6.89 (d, J=9.2 Hz, 2H), 3.89 (dd, J=11.6, 2.4 Hz, 1H), 3.54-3.64 (m, 3H), 3.46 (br d, J=11.6 Hz, 1H), 2.61 (td, J=11.6, 3.6 Hz, 1H), 2.29 (t, J=10.8 Hz, 1H), 1.14 (d, J=6.4 Hz, 3H).

### Step 2: Int. 430

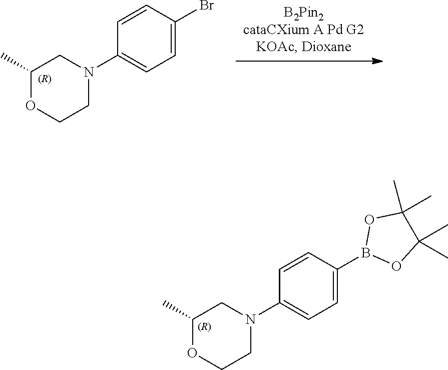

A mixture of (R)-4-(4-bromophenyl)-2-methylmorpholine (4 g, 15.62 mmol, 1 eq), 4,4,5,5-tetramethyl-2-(4,4,5,5-tetramethyl-1,3,2-dioxaborolan-2-yl)-1,3,2-dioxaborolane (7.93 g, 31.23 mmol, 2 eq), KOAc (3.83 g, 39.04 mmol, 2.5 eq), cataCXium A Pd G2 (1.04 g, 1.56 mmol, 0.1 eq) in dioxane (50 mL) was degassed with N₂ for 15 min. The reaction was heated to 90° C. for 2 h. After cooling to room temperature, the reaction mixture was concentrated. EtOAc (30 mL) was added, solids were filtered off, and the filtrate was concentrated under reduced pressure. The residue was purified by flash chromatography (SiO₂, 0-5% EtOAc in Pet. ether) to give Int. 430 (2.06 g, 6.20 mmol, 39.68%) as a gray solid. LCMS: (ESI+): m/z 304.2 (M+H⁺), ¹H NMR (400 MHz, DMSO-d₆): δ 7.52 (d, J=8.4 Hz, 2H), 6.90 (d, J=8.4 Hz, 2H), 3.90 (d, J=10.8 Hz, 1H), 3.54-3.69 (m, 4H), 2.66 (td, J=12.0, 3.2 Hz, 1H), 2.34 (t, J=10.8 Hz, 1H), 1.26 (s, 12H), 1.15 (d, J=6.4 Hz, 3H).

### Int. 431: Preparation of (S)-1-methyl-2-(4-(4,4,5,5-tetramethyl-1,3,2-dioxaborolan-2-yl)phenyl)piperidine

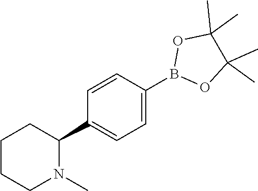 [SMILES: CN1CCCC[C@H]1c1ccc(B2OC(C)(C)C(C)(C)O2)cc1]

### Step 1: 2-(4-bromophenyl)-1-methylpiperidine

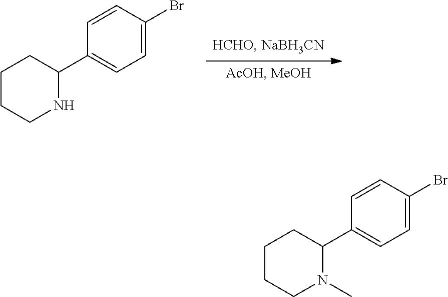

To a solution of 2-(4-bromophenyl)piperidine (2 g, 8.32 mmol, 1.0 eq) in MeOH (40 mL) was added 37% formaldehyde (3.38 g, 41.64 mmol, 3.10 mL, 5 eq) and AcOH (50.02 mg, 832.86 μmol, 47.68 μL, 0.1 eq) and stirred at 0° C. for 30 min. Then, NaBH₃CN (1.57 g, 24.98 mmol, 3 eq) was added and stirred at 0° C. for 30 min. Upon the consumption of starting material, the reaction mixture was diluted with water (50 mL) and extracted with EtOAc (40 mL×3). The combined organic layers were dried over Na₂SO₄, filtered, and concentrated under reduced pressure to give 2-(4-bromophenyl)-1-methylpiperidine (2.2 g, crude) as a yellow oil. The product was used without further purification. LCMS (ESI+): m/z 254.2 (M+H⁺). ¹H NMR (400 MHz, CDCl₃-d): δ7.48 (d, J=8.4 Hz, 2H), 7.22 (d, J=8.4 Hz, 2H), 3.20-3.10 (m, 1H), 2.92 (dd, J=2.8, 11.2 Hz, 1H), 2.32-2.22 (m, 1H), 2.08 (s, 3H), 1.91-1.84 (m, 1H), 1.80-1.76 (m, 2H), 1.75-1.70 (m, 1H), 1.70-1.60 (m, 1H), 1.48-1.35 (m, 1H).

### Step 2: 1-methyl-2-(4-(4,4,5,5-tetramethyl-1,3,2-dioxaborolan-2-yl)phenyl)piperidine

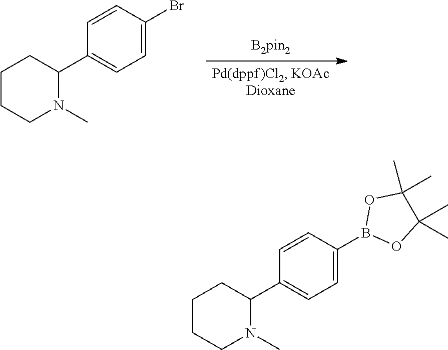 [SMILES: CN1CCCCC1c1ccc(Br)cc1 ; "B2pin2" ; "Pd(dppf)Cl2, KOAcDioxane" ; CN1CCCCC1c1ccc(B2OC(C)(C)C(C)(C)O2)cc1]

To a solution of 2-(4-bromophenyl)-1-methylpiperidine (1.1 g, 4.33 mmol, 1 eq) in dioxane (40 mL) was added B₂Pin₂ (1.65 g, 6.49 mmol, 1.5 eq), Pd(dppf)Cl₂ (316.67 mg, 432.79 μmol, 0.1 eq) and KOAc (849.50 mg, 8.66 mmol, 2 eq). The mixture was degassed with N₂ then stirred at 100° C. for 1 h under N₂. Then, the reaction mixture was diluted with water (50 mL) and extracted with EtOAc (40 mL×3). The combined organic layers were dried over Na₂SO₄, filtered, and concentrated under reduced pressure. The residue was purified by prep-HPLC (C18, H₂O+10 mM NH₄HCO₃ and MeCN) to afford 1-methyl-2-(4-(4,4,5,5-tetramethyl-1,3,2-dioxaborolan-2-yl)phenyl)piperidine (117 mg, 388.41 μmol, 8.97%) as a white solid. LCMS (ESI+): m/z 302.1 (M+H⁺). ¹H NMR (400 MHz, DMSO-d₆) δ 7.62 (d, J=7.6 Hz, 2H), 7.31 (d, J=7.6 Hz, 2H), 2.93 (d, J=11.2 Hz, 1H), 2.76 (d, J=9.2 Hz, 1H), 2.02 (t, J=10.4 Hz, 1H), 1.87 (s, 3H), 1.72 (d, J=12.4 Hz, 1H), 1.67-1.52 (m, 3H), 1.45-1.30 (m, 2H), 1.28 (s, 9H).

### Step 3: Int. 431

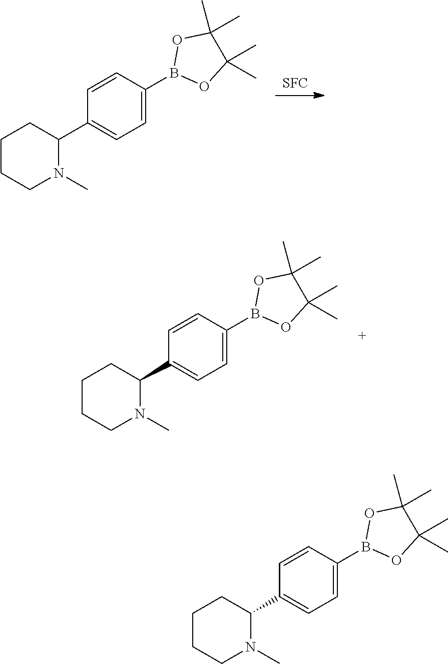

1-methyl-2-(4-(4,4,5,5-tetramethyl-1,3,2-dioxaborolan-2-yl)phenyl)piperidine was further separated by SFC (DAICEL CHIRALPAK AD, CO₂/MeOH+0.1% IPAm 10% isocratic) to afford Int. 431 (0.1 g, 331.97). LCMS (ESI+): m/z 302.1 (M+H⁺). ¹H NMR (400 MHz, CDCl₃-d) δ7.77 (d, J=8.0 Hz, 2H), 7.34 (d, J=7.6 Hz, 2H), 3.04 (br d, J=11.6 Hz, 1H), 2.78 (dd, J=2.4, 11.2 Hz, 1H), 2.17-2.06 (m, 1H), 2.00 (s, 3H), 1.83-1.68 (m, 4H), 1.64-1.52 (m, 1H), 1.38 (br d, J=4.4 Hz, 1H), 1.34 (s, 12H).

### Int. 433: Preparation of 4-bromo-6-chloro-1-methyl-1H-indazole

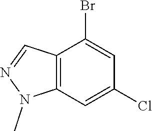

Int. 433 was synthesized using the procedure outlined in Int. 411. LCMS (ESI+): m/z 293.5 (M+H⁺).

### Int. 438: Preparation of (S)-3-(2-bromo-5-chlorophenyl)-2-((tert-butoxycarbonyl)amino)propanoic acid

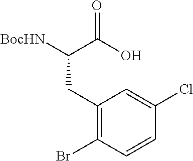 [SMILES: CC(C)(C)OC(=O)N[C@@H](Cc1cc(Cl)ccc1Br)C(=O)O]

### Step 1: methyl(S)-3-(2-bromo-5-chlorophenyl)-2-((tert-butoxycarbonyl)amino)propanoate

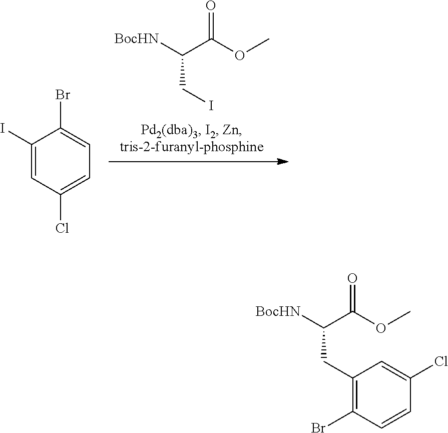

To a vial was added zinc (4.8 g, 3 Eq, 73 mmol). The vial was evacuated and backfilled with argon. DMA (10 mL) was added by syringe. Iodine (0.37 g, 0.06 Eq, 1.5 mmol) was added as a solid, and the mixture was stirred until exotherm and color change had subsided. Separately, methyl (R)-2-((tert-butoxycarbonyl)amino)-3-iodopropanoate (18 g, 2.3 Eq, 55 mmol) was dissolved in 10 mL DMA, and this solution was added by syringe to zinc/iodine mixture while stirring, forming the zincate reagent in situ.

Separately, 1-bromo-4-chloro-2-iodobenzene (7.7 g, 1 Eq, 24 mmol) was added to a flask, and the flask was evacuated and backfilled with argon. DMA (10 mL) was added to this flask by syringe. To this solution was added Pd₂(dba)₃ (0.67 g, 0.03 Eq, 0.73 mmol) and tris-2-furanyl-phosphine (1.0 g, 0.18 Eq, 4.4 mmol) as solids, and the mixture was stirred to dissolve. The zincate solution was added to the reaction flask by syringe while stirring. The reaction was heated to 55° C. and stirred overnight. The reaction was allowed to cool to room temperature, diluted with water, and extracted three times with EtOAc. The combined organics were washed with brine, dried over MgSO₄, filtered, and concentrated by rotary evaporation. The crude product was purified by silica gel chromatography (0 to 100% EtOAc in hexanes) to yield methyl(S)-3-(2-bromo-5-chlorophenyl)-2-((tert-butoxycarbonyl)amino)propanoate (3.93 g, 10.02 mmol, 41%). LCMS (ESI+): m/z 338.1 (M−55+H⁺).

### Step 2: Int. 438

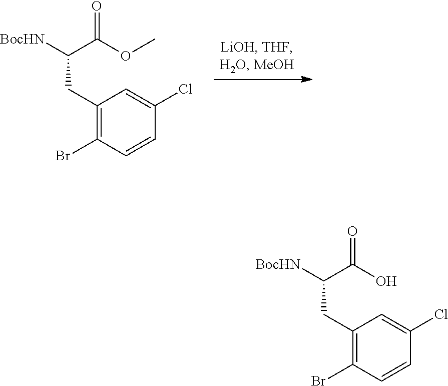

Methyl(S)-3-(2-bromo-5-chlorophenyl)-2-((tert-butoxycarbonyl)amino)propanoate (3.93 g, 1 Eq, 10 mmol) was dissolved in THF (4 mL), water (2 mL), and MeOH (1 mL). Lithium hydroxide monohydrate (480 mg, 2 Eq, 20 mmol) was added and the reaction mixture was stirred for 1 hour. The reaction was stirred for 1 hour, acidified with 1M HCl, and extracted three times with EtOAc. The combined organics were washed with brine, dried over MgSO₄, filtered, and concentrated by rotary evaporation. Int. 438 (3.78 g, 10 mmol, quant.) was used without further purification. LCMS (ESI+): m/z 378.2 (M−55+H⁺).

### A. Representative Procedures for Preparing Compounds of Formula (I)

The following subsections describe procedures for preparing compounds of Formula (I). It is understood that compounds of Formula (I) can be prepared using variety of synthetic routes. The following procedures are meant to be illustrative, not limiting. Generally, compounds of Formula (I) are prepared by synthesizing a macrocyclic portion of the compound and completing final synthetic steps to obtain compounds of Formula (I). The final synthetic steps include optional modifications (e.g., reduction, Phe Suzuki coupling, acylation) and tail addition (comprising the R³, R^{4a/b/c}, and sometimes R^{5a/b/c} variable positions of Formula (I)). The macrocyclic synthesis procedures described herein include Methods A, B, C, and D. The final synthesis procedures described herein include Methods 1-14.

### 1. Macrocycle Synthesis Procedures

a. Method A

### Generic Procedure for Method A

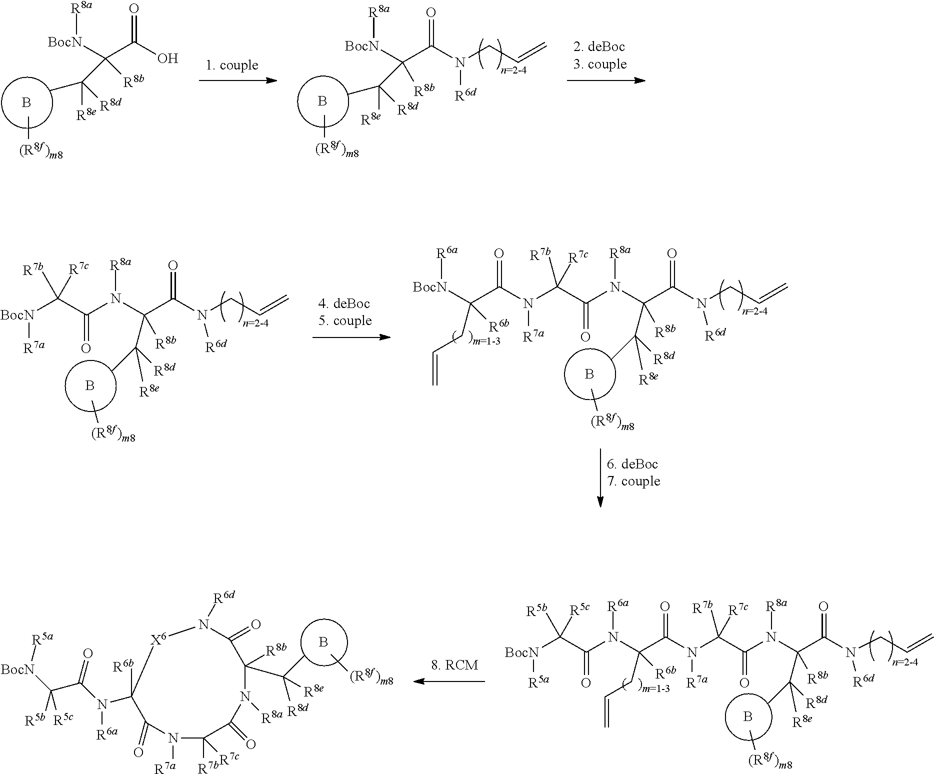

### Representative Procedure for Method A

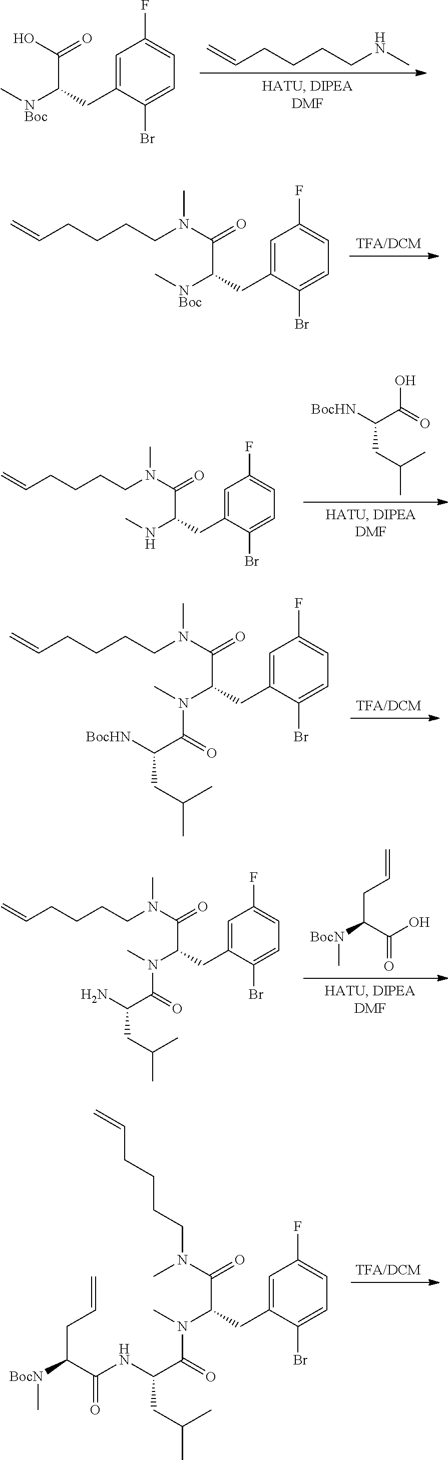

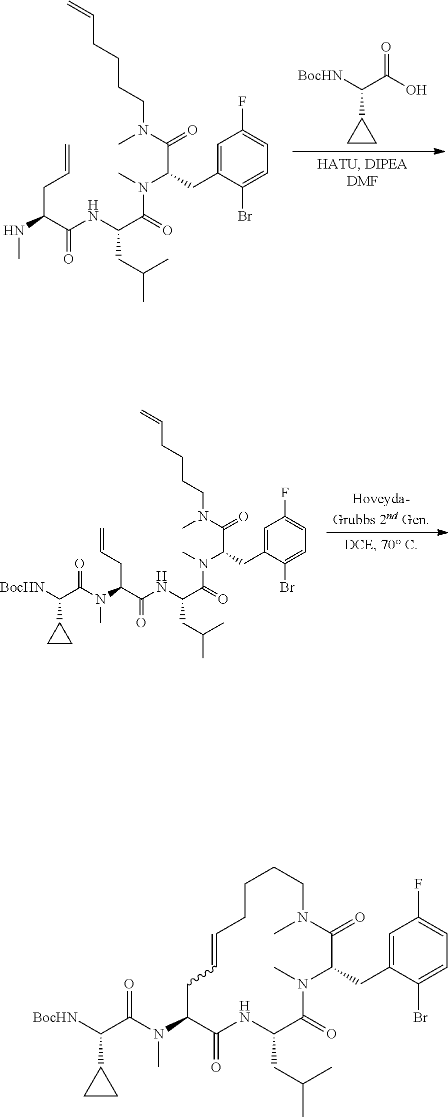

### Representative Experimental for Method A:

### Step 1: (S)-3-(2-bromo-5-fluorophenyl)-2-((tert-butoxycarbonyl)(methyl)amino)propanoic acid (201)

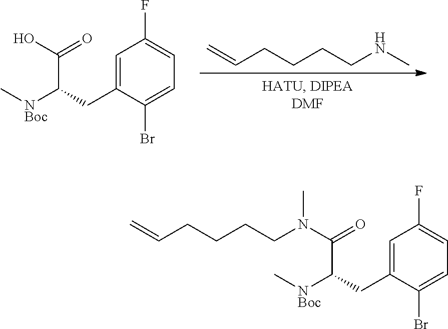

To a solution of Int. 31 (32 g, 85 mmol, 1 eq) and Int. 3 (11.5 g, 112 mmol, 1.3 eq) in DMF (1000 mL) was added HATU (48.5 g, 127.5 mmol, 1.5 eq) and DIPEA (55.0 g, 425.3 mmol, 74.1 mL, 5 eq) at 0° C. The mixture was removed from the cooling bath and stirred at 25° C. until LCMS analysis showed the complete consumption of starting material. The reaction was added into ice water 2000 mL, then extracted with EtOAc (2000 mL×2), the combined organic layers were washed with brine, dried over MgSO₄ and filtered. The filtrate was concentrated under reduced pressure to give crude product which was purified by column chromatography (SiO₂, EtOAc:Hexanes; 0-60%) to yield 201: (37 g, 78.5 mmol, 92.3% yield) as a yellow oil. LCMS (ESI+): m/z 493.1 (M+Na⁺)

### Step 2: (S)-3-(2-bromo-5-fluorophenyl)-N-(hex-5-en-1-yl)-N-methyl-2-(methylamino)propenamide TFA salt (202)

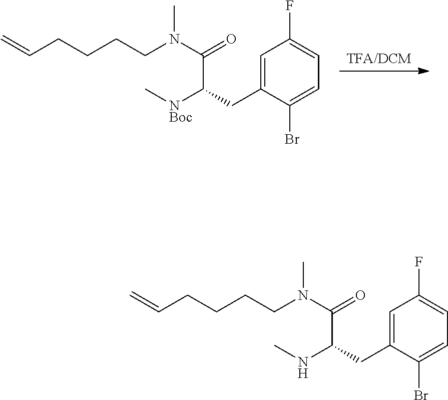 [SMILES: C=CCCCCN(C)C(=O)[C@H](Cc1cc(F)ccc1Br)N(C)C(=O)OC(C)(C)C ; "TFA/DCM" ; C=CCCCCN(C)C(=O)[C@H](Cc1cc(F)ccc1Br)NC]

To a solution of 201 (37 g, 78.5 mmol, 1 eq) in DCM (400 mL) was added TFA (80 mL) at 0° C. The mixture was removed from the cooling bath and stirred at 25° C. until LCMS analysis showed the complete consumption of starting material. The reaction mixture was concentrated under reduced pressure to give 202 (38 g, crude, TFA) as a light-yellow oil. LCMS (ESI+): m/z 371.1 (M+H⁺)

### Step 3: Tert-butyl ((S)-1-(((S)-3-(2-bromo-5-fluorophenyl)-1-(hex-5-en-1-yl(methyl)amino)-1-oxopropan-2-yl)(methyl)amino)-4-methyl-1-oxopentan-2-yl)carbamate (203)

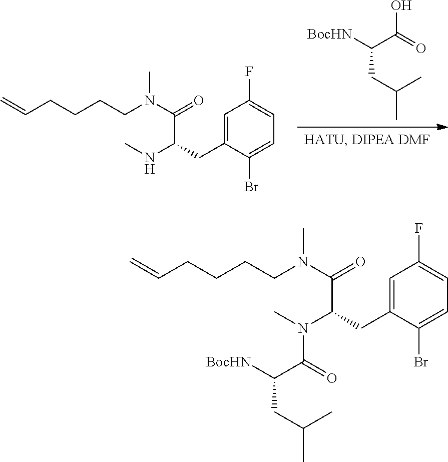

To a solution of 202 (38 g, 78.3 mmol, 1 eq, TFA salt) and Int. 151 (19.9 g, 86. mmol, 1.1 eq) in DMF (1000 mL) was added HATU (44.7 g, 117.5 mmol, 1.5 eq) and DIEPA (50.6 g, 391.5 mmol, 68.2 mL, 5 eq) at 0° C. The mixture was removed from the cooling bath and stirred at 25° C. until LCMS analysis showed the complete consumption of starting material. The reaction was added into ice water 2000 mL, then extracted with EtOAc (2000 mL×2), the combined organic layers were washed with brine, dried over MgSO₄ and filtered. The filtrate was concentrated under reduced pressure to give crude product which was purified by column chromatography (SiO₂, EtOAc:Hexanes 0-50%) to give 203 (45 g) as a yellow oil. LCMS (ESI+): m/z 606.2 (M+Na⁺)

### Step 4: (S)-2-amino-N—((S)-3-(2-bromo-5-fluorophenyl)-1-(hex-5-en-1-yl(methyl)amino)-1-oxopropan-2-yl)-N,4-dimethylpentanamide TFA salt (204)

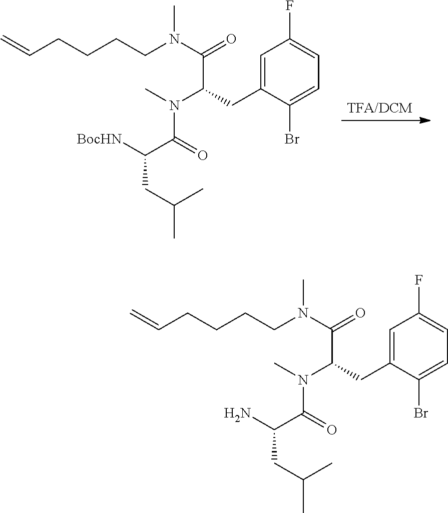 [SMILES: C=CCCCCN(C)C(=O)[C@H](Cc1cc(F)ccc1Br)N(C)C(=O)[C@H](CC(C)C)NC(=O)OC(C)(C)C ; "TFA/DCM" ; C=CCCCCN(C)C(=O)[C@H](Cc1cc(F)ccc1Br)N(C)C(=O)[C@@H](N)CC(C)C]

To a solution of 203 (45 g, 76.9 mmol, 1 eq) in DCM (500 mL) was added TFA (100 mL) at 0° C. The mixture was removed from the cooling bath and stirred at 25° C. until LCMS analysis showed the complete consumption of starting material. The reaction mixture was concentrated under reduced pressure to give 204 (46 g, crude) as a light-yellow oil. LCMS (ESI+): m/z 484.1 (M+H⁺)

### Step 5: Tert-butyl ((S)-1-(((S)-1-(((S)-3-(2-bromo-5-fluorophenyl)-1-(hex-5-en-1-yl(methyl)amino)-1-oxopropan-2-yl)(methyl)amino)-4-methyl-1-oxopentan-2-yl)amino)-1-oxopent-4-en-2-yl)(methyl)carbamate (205)

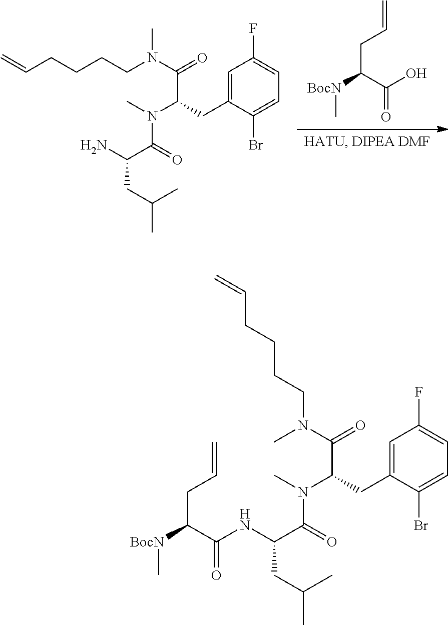

To a solution of 204 (46 g, 76.9 mmol, 1 eq, TFA) and Int. 152 (19.38 g, 84.6 mmol, 1.1 eq) in DMF (1300 mL) was added HATU (43.8 g, 115.3 mmol, 1.5 eq) and DIEPA (49.7 g, 384.32 mmol, 66.94 mL, 5 eq) at 0° C. The mixture was removed from the cooling bath and stirred at 25° C. until LCMS analysis showed the complete consumption of starting material. The reaction was added into ice water 2000 mL, then extracted with EtOAc (2000 mL×2), the combined organic layers were washed with brine, dried over MgSO₄ and filtered. The filtrate was concentrated under reduced pressure to give crude product which was purified by column chromatography (SiO₂, EtOAc:Hexanes; 0-60%) to give 205 (46 g, 66.1 mmol, 78% yield) as a yellow oil. LCMS (ESI+): m/z 717.4 (M+Na⁺)

### Step 6: (S)—N—((S)-1-(((S)-3-(2-bromo-5-fluorophenyl)-1-(hex-5-en-1-yl(methyl)amino)-1-oxopropan-2-yl)(methyl)amino)-4-methyl-1-oxopentan-2-yl)-2-(methylamino)pent-4-enamide TFA salt (206)

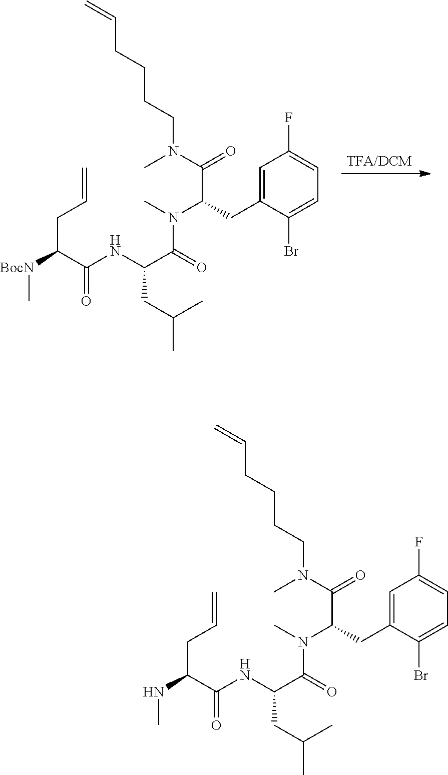 [SMILES: C=CCCCCN(C)C(=O)[C@H](Cc1cc(F)ccc1Br)N(C)C(=O)[C@H](CC(C)C)NC(=O)[C@H](CC=C)N(C)C(=O)OC(C)(C)C ; "TFA/DCM" ; C=CCCCCN(C)C(=O)[C@H](Cc1cc(F)ccc1Br)N(C)C(=O)[C@H](CC(C)C)NC(=O)[C@H](CC=C)NC]

To a solution of 205 (46 g, 66.1 mmol, 1 eq) in DCM (500 mL) was added TFA (100 mL) at 0° C. The mixture was removed from the cooling bath and stirred at 25° C. until LCMS analysis showed the complete consumption of starting material. The reaction mixture was concentrated under reduced pressure to give 206 (47 g) as a yellow oil. LCMS (ESI+): m/z 595.1 (M+H⁺) Step 7: Tert-butyl((1S,4S,7S,10S)-4-allyl-10-(2-bromo-5-fluorobenzyl)-1-cyclopropyl-7-isobutyl-3,9,12-trimethyl-2,5,8,11-tetraoxo-3,6,9,12-tetraazaoctadec-17-en-1-yl)carbamate (207)

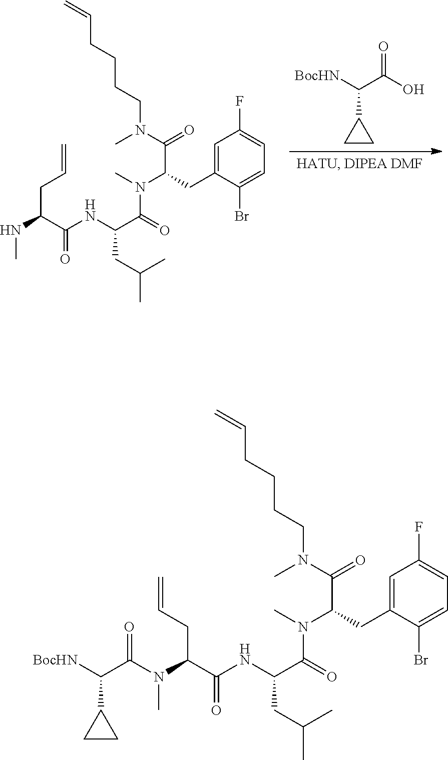

To a solution 206 (47 g, 66.2 mmol, 1 eq, TFA) and Int. 153 (16.4 g, 76.2 mmol, 1.15 eq) in DMF (1300 mL) was added HATU (37.8 g, 99.4 mmol, 1.5 eq) and DIEPA (85.6 g, 662.3 mmol, 115.4 mL, 10 eq) at 0° C. The mixture was removed from the cooling bath and stirred at 25° C. until LCMS analysis showed the complete consumption of starting material. The reaction was added into ice water 2000 mL, then extracted with EtOAc (2000 mL×2), the combined organic layers were washed with brine, dried over MgSO₄ and filtered. The filtrate was concentrated under reduced pressure to give crude product which was purified by column chromatography (SiO₂, EtOAc:Hexanes; 0-60%) to give 207 (44 g, 55.5 mmol, 70% yield) as a yellow oil. LCMS (ESI+): m/z 814.5 (M+Na⁺). ¹H NMR (400 MHz, DMSO-d₆) δ 0.34 (br s, 2H) 0.42 (br d, J=7.87 Hz, 2H) 0.85 (br d, J=6.32 Hz, 6H) 1.11 (br d, J=7.15 Hz, 1H) 1.29 (br d, J=6.44 Hz, 3H) 1.38 (s, 9H) 1.45 (br s, 3H) 1.52-1.63 (m, 1H) 2.02 (br d, J=6.68 Hz, 2H) 2.30-2.40 (m, 1H) 2.83 (s, 3H) 2.94 (br s, 2H) 3.00 (s, 3H) 3.03 (br d, J=9.06 Hz, 1H) 3.06 (s, 3H) 3.09-3.24 (m, 2H) 3.26-3.47 (m, 1H) 4.14 (br s, 1H) 4.66-4.76 (m, 1H) 4.81-5.23 (m, 5H) 5.67 (br s, 1H) 5.70-5.85 (m, 2H) 6.21-6.69 (m, 1H) 6.92-7.01 (m, 1H) 7.03-7.15 (m, 1H) 7.54 (br dd, J=8.52, 5.54 Hz, 1H)

### Step 8: Tert-butyl ((S)-2-(((3S,6S,9S)-3-(2-bromo-5-fluorobenzyl)-6-isobutyl-1,4-dimethyl-2,5,8-trioxo-1,4,7-triazacyclohexadec-11-en-9-yl)(methyl)amino)-1-cyclopropyl-2-oxoethyl)carbamate (208)

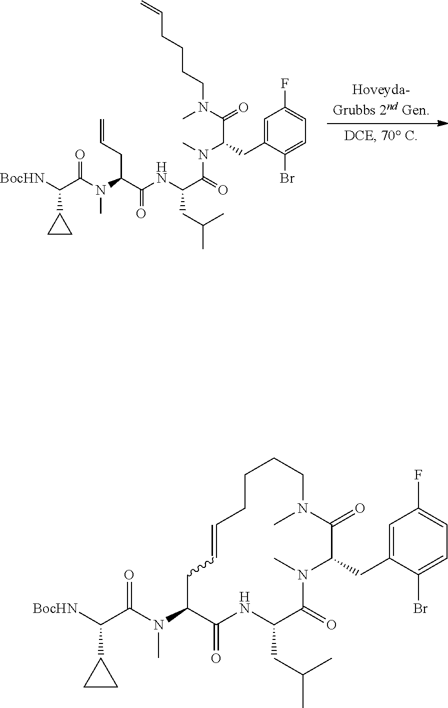 [SMILES: C=CCCCCN(C)C(=O)[C@H](Cc1cc(F)ccc1Br)N(C)C(=O)[C@H](CC(C)C)NC(=O)[C@H](CC=C)N(C)C(=O)[C@@H](NC(=O)OC(C)(C)C)C1CC1 ; "DCE, 70° C." ; "Hoveyda-Grubbs 2nd Gen." ; CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(F)ccc2Br)N(C)C1=O]

A mixture the pre-cyclized peptide 207 (8 g, 10.1 mmol, 1 eq), [1,3-bis(2,4,6-trimethylphenyl)imidazolidin-2-ylidene]-dichloro-[(2-isopropoxyphenyl)methylene]ruthenium (695.5 mg, 1.11 mmol, 0.11 eq) in DCE (1600 mL) was degassed and purged with N₂ for 0.5 hr. The mixture was capped and stirred at 70° C. until LCMS analysis showed the complete consumption of starting material. The reaction mixture was concentrated under reduced pressure to give a thick residue. The crude product was purified by reversed phase HPLC flash C18 gel chromatography (ISCO; 330 g SepaFlash C18 Flash Column, eluent of 50-100% MeCN in H₂O buffered with 0.1% TFA) 208 (5.41 g, 6.8 mmol, 67% yield) as an off white solid. Compound was isolated as a mix of cis/trans isomers LCMS (ESI+): m/z 764.4 (M+H). ¹H NMR of major isomer (400 MHz, DMSO-d₆) δ 0.25-0.47 (m, 4H) 0.70-0.86 (m, 6H) 1.05-1.13 (m, 1H) 1.29 (br s, 3H) 1.40 (br s, 9H) 1.43-1.62 (m, 3H) 1.78-2.16 (m, 3H) 2.47-2.49 (m, 1H) 2.51-2.56 (m, 1H) 2.60-3.03 (m, 11H) 3.09-3.29 (m, 2H) 4.10-4.32 (m, 2H) 4.53-5.07 (m, 2H) 5.19-5.40 (m, 1H) 5.41-5.58 (m, 1H) 6.25-6.60 (m, 1H) 6.95-7.06 (m, 1H) 7.07-7.16 (m, 1H) 7.53-7.63 (m, 1H) 7.74-7.98 (m, 1H)

b. Method B

### Generic Procedure for Method B: Z-Selective Macrocycle Formation

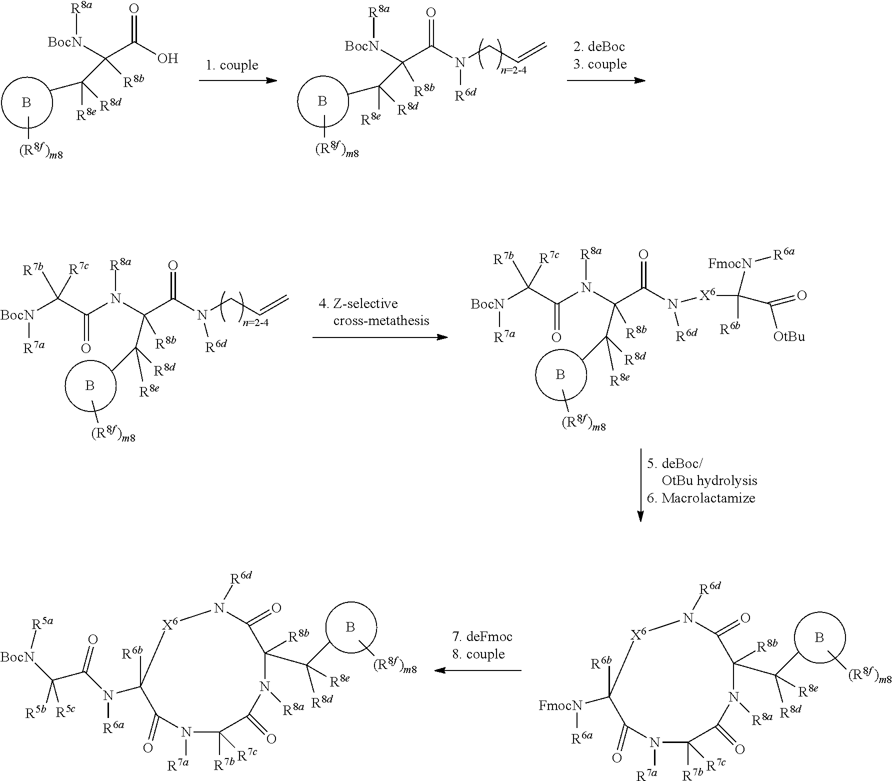

### Representative Procedure for Method B

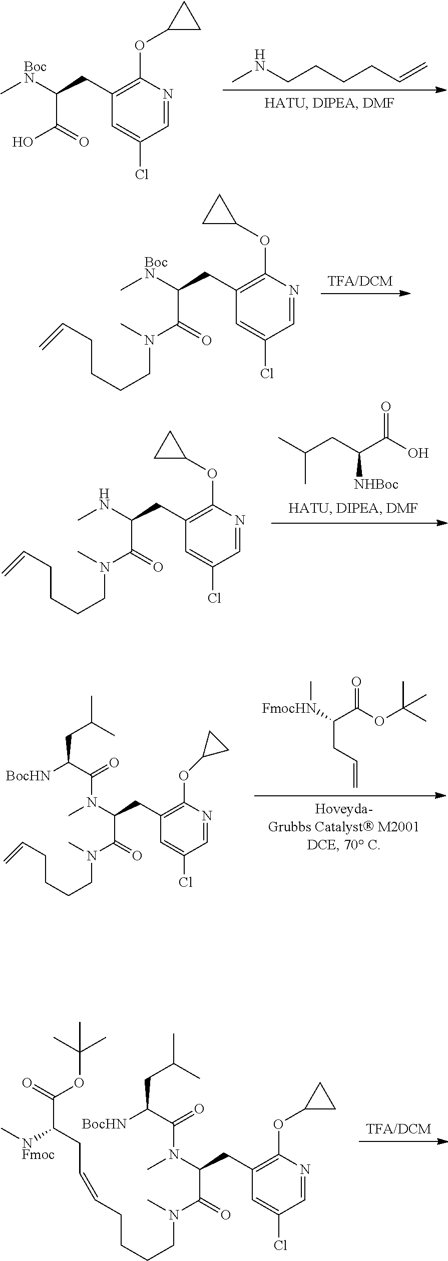

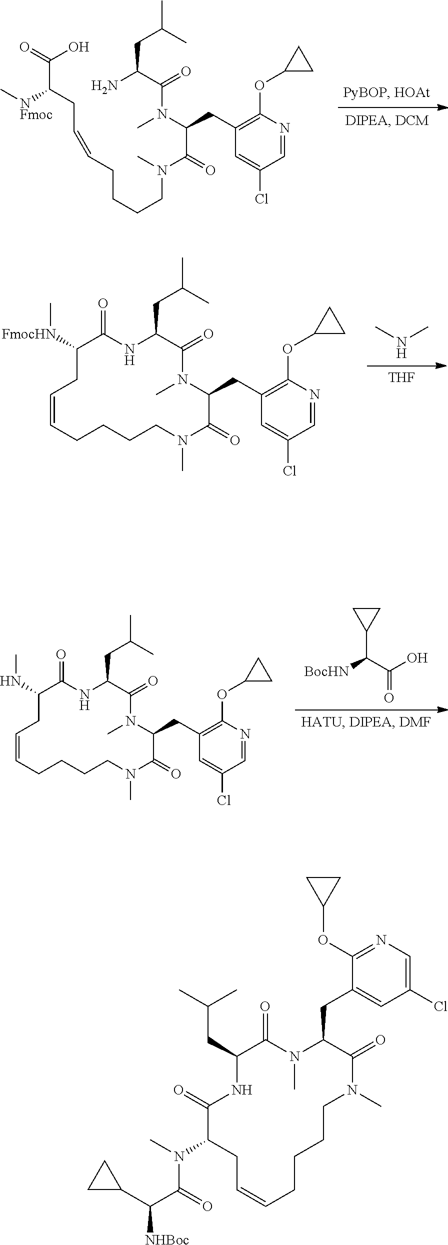

### Representative Experimental for Method B:

### Step 1: Tert-butyl (S)-(3-(5-chloro-2-cyclopropoxypyridin-3-yl)-1-(hex-5-en-1-yl(methyl)amino)-1-oxopropan-2-yl)(methyl)carbamate (209)

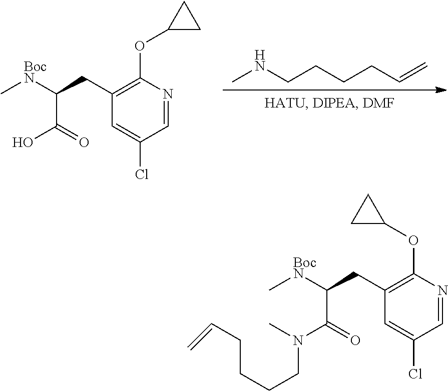

To a solution of Int. 7 (7.0 g, 1 Eq, 19 mmol) and Int. 3 (2.8 g, 1.3 Eq, 25 mmol) in DMF (94 mL) was added HATU (9.3 g, 1.3 Eq, 25 mmol) and DIPEA (9.8 g, 13 mL, 4 Eq, 76 mmol) at 0° C. The mixture was removed from the cooling bath and stirred at 25° C. until LCMS analysis showed the complete consumption of starting material. The reaction was added into ice water 400 mL, then extracted with EtOAc (300 mL×2), the combined organic layers were washed with brine, dried over MgSO₄ and filtered. The filtrate was concentrated under reduced pressure to give crude product which was purified by column chromatography (SiO2, EtOAc:Hexanes; 0-60%) to yield 209 (8.0 g, 17 mmol, 91% yield) as a yellow oil. LCMS (ESI+): m/z 466.4 (M+H⁺)

### Step 2: (S)-3-(5-chloro-2-cyclopropoxypyridin-3-yl)-N-(hex-5-en-1-yl)-N-methyl-2-(methylamino)propanamide TFA salt (210)

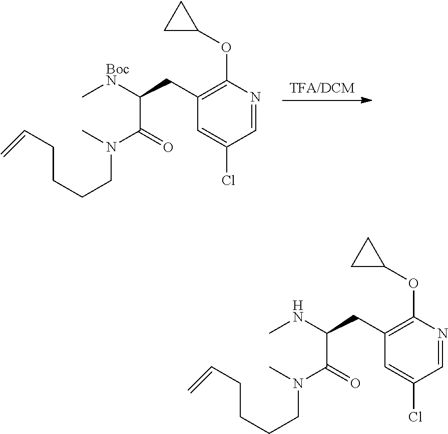 [SMILES: C=CCCCCN(C)C(=O)[C@H](Cc1cc(Cl)cnc1OC1CC1)N(C)C(=O)OC(C)(C)C ; "TFA/DCM" ; C=CCCCCN(C)C(=O)[C@H](Cc1cc(Cl)cnc1OC1CC1)NC]

To a solution of 209 (8.0 g, 17 mmol, 1 eq) in DCM (80 mL) was added TFA (16 mL) at 0° C. The mixture was removed from the cooling bath and stirred at 25° C. until LCMS analysis showed the complete consumption of starting material. The reaction mixture was concentrated under reduced pressure to give 210 (8.8 g, crude, TFA) as a light-yellow oil. LCMS (ESI+): m/z 366.2 (M+H⁺)

### Step 3: Tert-butyl ((S)-1-(((S)-3-(5-chloro-2-cyclopropoxypyridin-3-yl)-1-(hex-5-en-1-yl(methyl)amino)-1-oxopropan-2-yl)(methyl)amino)-4-methyl-1-oxopentan-2-yl)carbamate (211)

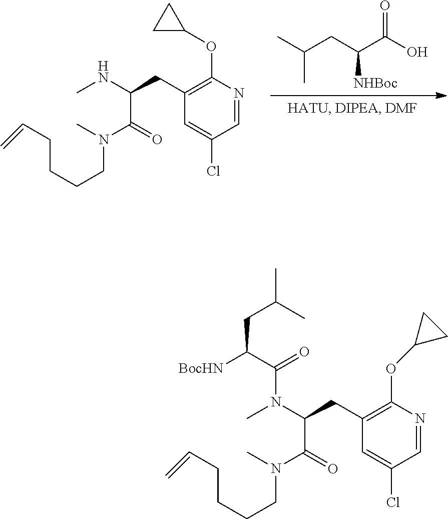

To a solution of 210 (8.0 g, 17 mmol, 1 eq, TFA) and Int. 151 (5.1 g, 1.2 Eq, 21 mmol) in DMF (86 mL) was added DIPEA (8.9 g, 12 mL, 4 Eq, 69 mmol) and HATU (7.8 g, 1.2 Eq, 21 mmol) at 0° C. The mixture was removed from the cooling bath and stirred at 25° C. until LCMS analysis showed the complete consumption of starting material. The reaction was added into ice water 400 mL, then extracted with EtOAc (300 mL×2), the combined organic layers were washed with brine, dried over MgSO₄ and filtered. The filtrate was concentrated under reduced pressure to give crude product which was purified by column chromatography (SiO₂, EtOAc:Hexanes; 0-100%) to yield 211 (9.2 g, 16 mmol, 93% yield) as a yellow oil. LCMS (ESI+): m/z 579.7 (M+H⁺)

### Step 4: Tert-butyl (6S,9S,19S,Z)-19-((((9H-fluoren-9-yl)methoxy)carbonyl)(methyl)amino)-9-((5-chloro-2-cyclopropoxypyridin-3-yl)methyl)-6-isobutyl-2,2,8,11-tetramethyl-4,7,10-trioxo-3-oxa-5,8,11-triazaicos-16-en-20-oate (212)

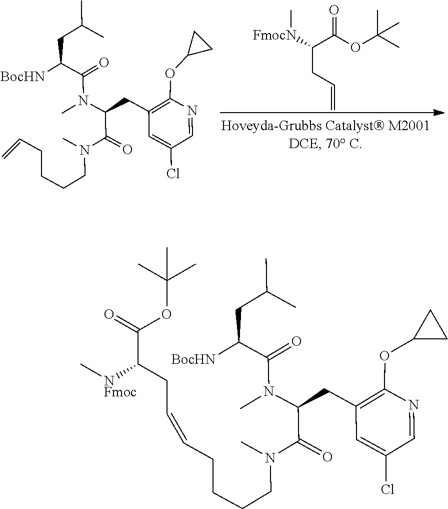

A mixture of Int. 154 (2.5 g, 1 Eq, 4.3 mmol) 211 (5.3 g, 3 Eq, 13 mmol) in DCE (22 mL) was degassed and purged with Ar for 0.5 hr, and Hoveyda-Grubbs Catalyst® M2001 (0.41 g, 0.15 Eq, 0.65 mmol) was added to the mixture. The mixture was stirred at 70° C. until LCMS analysis showed the complete consumption of starting material. The reaction mixture was concentrated under reduced pressure to give a thick residue. The crude product was purified by column chromatography (SiO₂, EtOAc:Hexanes; 0-100%) to give 212 (1.8 g, 1.9 mmol, 44% yield, crude, Trans/Cis=1:4) as brown syrup. LCMS (ESI+): m/z 958.8 (M+H).

### Step 5: (S,Z)-2-((((9H-fluoren-9-yl)methoxy)carbonyl)(methyl)amino)-9-((S)-2-((S)-2-(12-azaneyl)-N,4-dimethylpentanamido)-3-(5-chloro-2-cyclopropoxypyridin-3-yl)-N-methylpropanamido)non-4-enoic acid TFA salt (213)

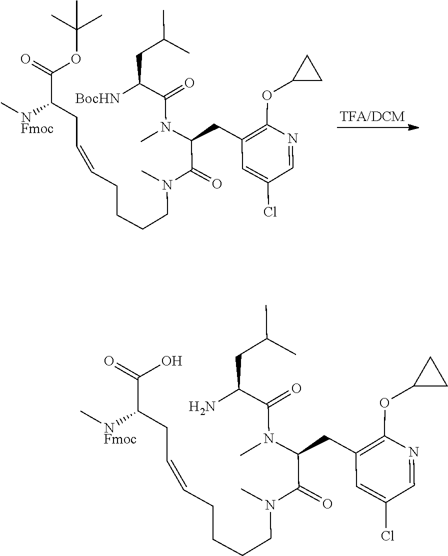

To a solution of peptide 212 (1.8 g, 1.9 mmol, 1 eq) in DCM (15 mL) was added TFA (7 mL) at 0° C. The mixture was removed from the cooling bath and stirred at 25° C. until LCMS analysis showed the complete consumption of starting material. The reaction mixture was concentrated under reduced pressure to give 213 (2 g, crude, TFA) as a brown oil. LCMS (ESI+): m/z 858.6 (M+H⁺)

### Step 6: (9H-fluoren-9-yl)methyl((3S,6S,9S,Z)-3-((5-chloro-2-cyclopropoxypyridin-3-yl)methyl)-6-isobutyl-1,4-dimethyl-2,5,8-trioxo-1,4,7-triazacyclohexadec-11-en-9-yl)(methyl)carbamate (214)

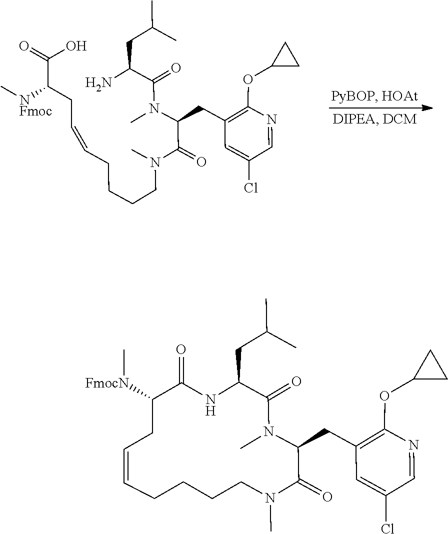

To a solution of the deprotected peptide 213 (1.8 g, 1.9 mmol, 1 eq, TFA) in DCM (0.37 L), add DIEPA (0.87 g, 1.2 mL, 3 Eq, 6.7 mmol). Separately, dissolve 1-Hydroxy-7-azabenzotriazole (0.37 g, 0.38 mL, 1.2 Eq, 2.7 mmol) and PyBOP (1.4 g, 1.2 Eq, 2.7 mmol) in DCM (0.37 L) and add DIEPA (0.87 g, 1.2 mL, 3 Eq, 6.7 mmol). Add the second solution into the first solution dropwise by additional funnel. The mixture was stirred at 25° C. until LCMS analysis showed the complete consumption of starting material. The reaction mixture was concentrated under reduced pressure to give a thick residue. The crude product was purified by column chromatography (SiO₂, EtOAc:Hexanes; 0-100%) to give 214 (0.5 g, 0.6 mmol, 30% yield, 99% purity) as brown solid. LCMS (ESI+): m/z 784.5 (M+H⁺)

### Step 7: (3S,6S,9S,Z)-3-((5-chloro-2-cyclopropoxypyridin-3-yl)methyl)-6-isobutyl-1,4-dimethyl-9-(methylamino)-1,4,7-triazacyclohexadec-11-ene-2,5,8-trione (215)

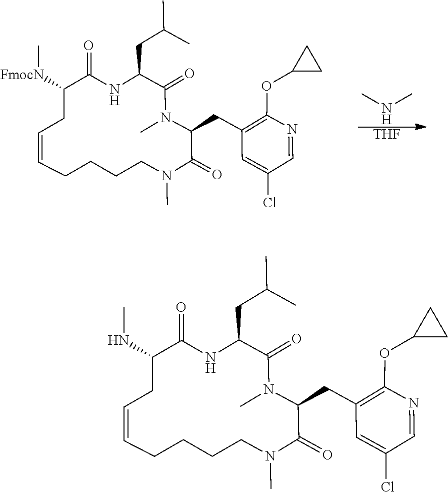

To peptide 214 (0.5 g, 0.6 mmol, 1 eq) was added dimethylamine in THF (6.76 g, 10.4 mL, 2.0 molar, 32.6 Eq, 20.8 mmol) at 0° C. The mixture was removed from the cooling bath and stirred at 25° C. until LCMS analysis showed the complete consumption of starting material. The reaction mixture was concentrated under reduced pressure to give a thick residue. The crude product was purified by reversed phase HPLC flash C18 gel chromatography (ISCO; 12 g C18 Flash Column, eluent of 20-100% MeCN in H₂O buffered with 0.1% TFA) to give 215 (0.35 g, 0.64 mmol, 100% yield) LCMS (ESI+): m/z 562.5 (M+H⁺)

### Step 8: Tert-butyl ((S)-2-(((3S,6S,9S,Z)-3-((5-chloro-2-cyclopropoxypyridin-3-yl)methyl)-6-isobutyl-1,4-dimethyl-2,5,8-trioxo-1,4,7-triazacyclohexadec-11-en-9-yl)(methyl)amino)-1-cyclopropyl-2-oxoethyl)carbamate (216)

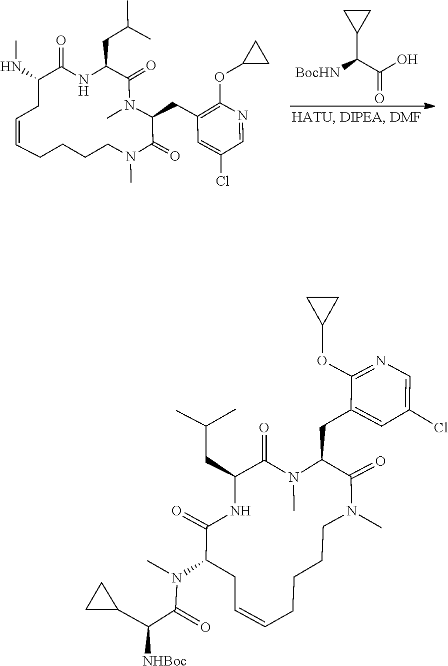 [SMILES: CN[C@H]1C/C=C\CCCCN(C)C(=O)[C@H](Cc2cc(Cl)cnc2OC2CC2)N(C)C(=O)[C@H](CC(C)C)NC1=O ; CC(C)(C)OC(=O)N[C@H](C(=O)O)C1CC1 ; CC(C)C[C@@H]1NC(=O)[C@@H](N(C)C(=O)[C@@H](NC(=O)OC(C)(C)C)C2CC2)C/C=C\CCCCN(C)C(=O)[C@H](Cc2cc(Cl)cnc2OC2CC2)N(C)C1=O]

To a solution of deprotected starting material 215 (350 mg, 0.64 mmol, 1 eq) and Int. 153 (164 mg, 1.2 Eq, 764 μmol) in DMF (3.18 mL) was added DIPEA (494 mg, 666 μL, 6 Eq, 3.82 mmol) and HATU (291 mg, 1.2 Eq, 764 μmol) at 0° C. The mixture was removed from the cooling bath and stirred at 25° C. until LCMS analysis showed the complete consumption of starting material. The mixture was neutralized to pH˜7 with 1N HCl and directly purified by RP-HPLC (C18, 50% to 80% MeCN/H₂O with 0.5% formic acid) to yield 216 (120 mg, 158 μmol, 25%) as off-white solid. LCMS (ESI+): m/z 759.5 (M+H⁺)

c. Method C

### Generic Procedure for Method C

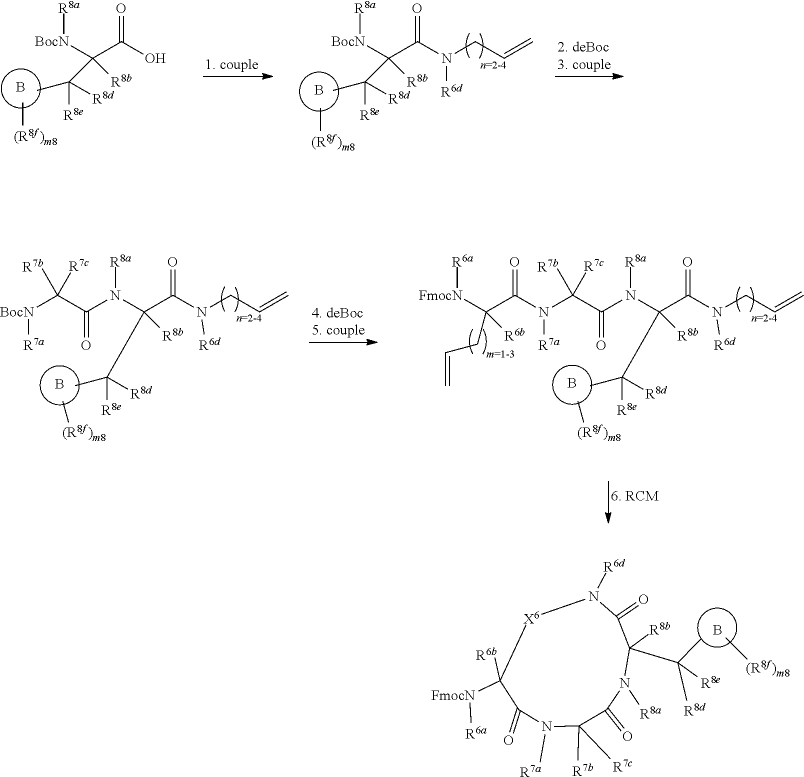

As seen when comparing Method A and Method C, Steps 1-5 follow similar synthetic procedures.

### Representative Experimental for Method C:

### (9H-fluoren-9-yl)methyl((S)-1-(((S)-1-(((S)-3-(1,4-dichloro-115-chlorinin-3-yl)-1-(hex-5-en-1-ylamino)-1-oxopropan-2-yl)(methyl)amino)-4-methyl-1-oxopentan-2-yl)amino)-1-oxopent-4-en-2-yl)carbamate (217)

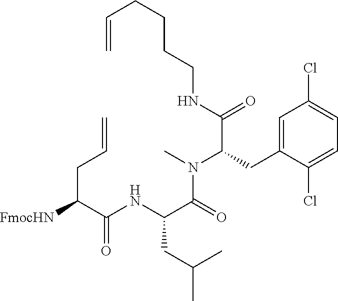

Compound 217 was prepared according to Steps 1-5 of Method A with starting materials Int. 1, 6, 151 and 21. LCMS (ESI+): m/z 761.1 (M+H).

### Step 6: (9H-fluoren-9-yl)methyl((3S,6S,9S)-3-(2,5-dichlorobenzyl)-6-isobutyl-4-methyl-2,5,8-trioxo-1,4,7-triazacyclohexadec-11-en-9-yl)carbamate (218)

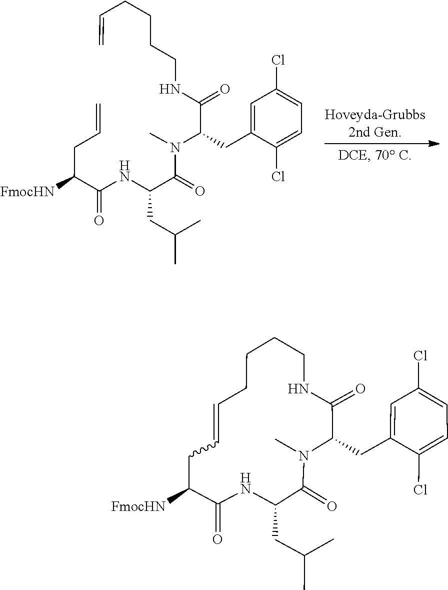

217 (200 mg, 1 Eq, 263 μmol) was dissolved in DCE (80 mL) and degassed for 30 min with a stream of nitrogen. Dichloro(1,3-dimesityl-2-imidazolidinylidene)(2-isopropoxybenzylidene)ruthenium (32 mg, 0.19 Eq, 51 μmol) was added rapidly and the reaction was degassed for an additional 10 min. reaction was then heated to 50° C. and continued until the complete consumption of starting material (monitored by LCMS). The reaction was cooled, concentrated, and purified by normal phase purification Methanol/DCM (0 to 15%, over 10 min.). 218 (121 mg, 165 μmol, 63%) was isolated as a brown solid. It was used as a mixture of E/Z isomers (˜7:3). LCMS (ESI+): m/z 733.4 (M+H).

d. Method D

### Generic Procedure for Method D

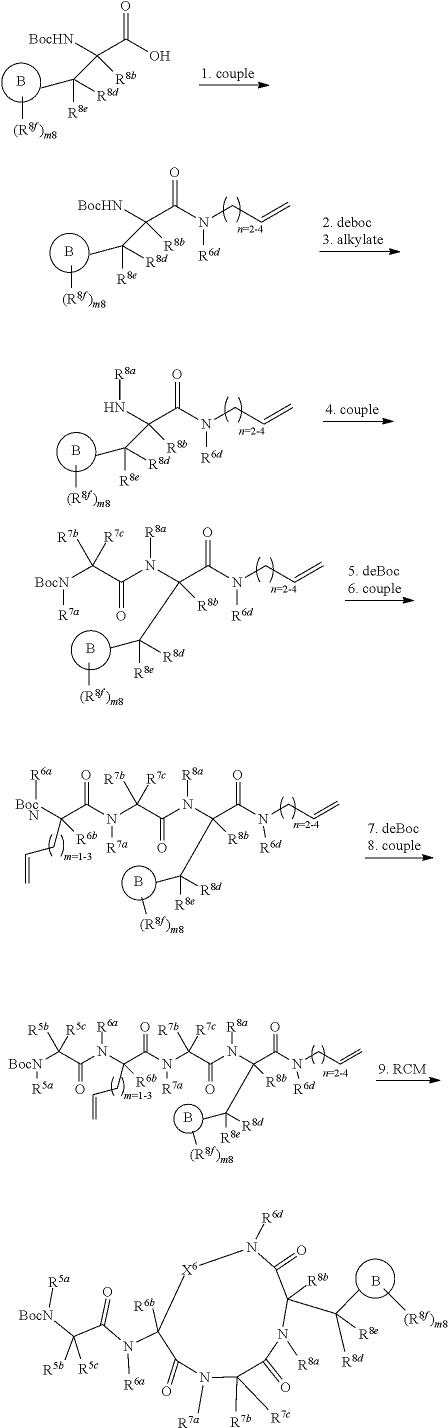

### Representative Procedure for Method D

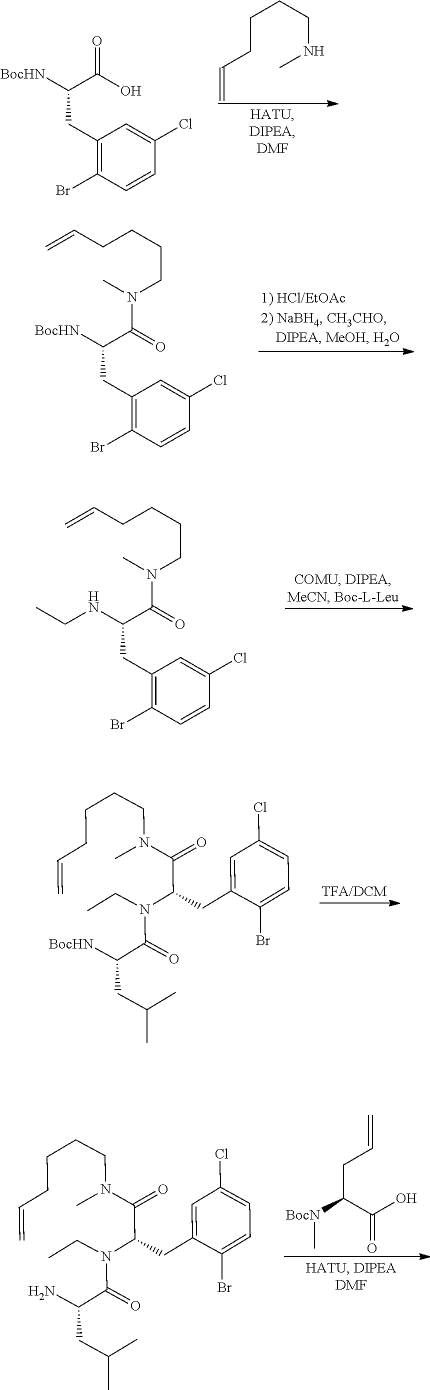

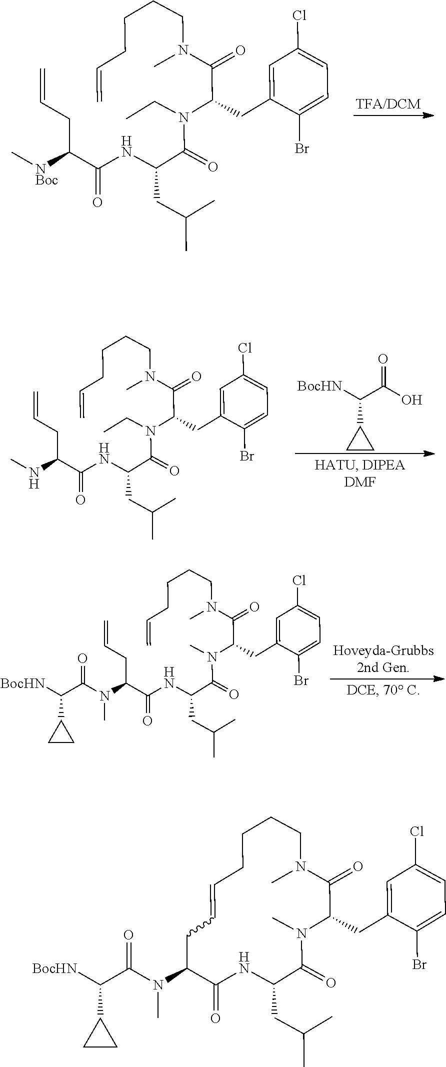 [SMILES: C=CCCCCN(C)C(=O)[C@H](Cc1cc(Cl)ccc1Br)N(CC)C(=O)[C@H](CC(C)C)NC(=O)[C@H](CC=C)N(C)C(=O)OC(C)(C)C ; C=CCCCCN(C)C(=O)[C@H](Cc1cc(Cl)ccc1Br)N(CC)C(=O)[C@H](CC(C)C)NC(=O)[C@H](CC=C)NC ; CC(C)(C)OC(=O)N[C@H](C(=O)O)C1CC1 ; C=CCCCCN(C)C(=O)[C@H](Cc1cc(Cl)ccc1Br)N(C)C(=O)[C@H](CC(C)C)NC(=O)[C@H](CC=C)N(C)C(=O)[C@@H](NC(=O)OC(C)(C)C)C1CC1 ; CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(Cl)ccc2Br)N(C)C1=O]

### Representative Experimental for Method D:

### Step 1: tert-butyl (S)-(3-(2-bromo-5-chlorophenyl)-1-(hex-5-en-1-yl(methyl)amino)-1-oxopropan-2-yl)carbamate (249)

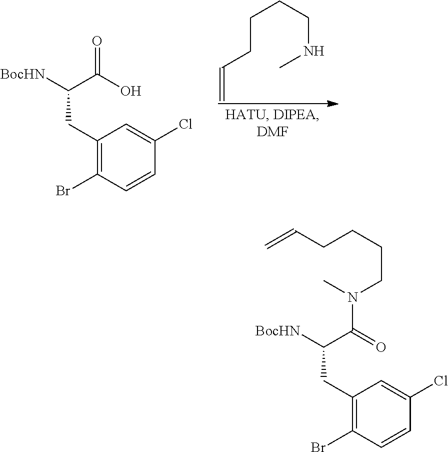

Int. 238 (3.73 g, 1 Eq, 10.02 mmol) was dissolved in DMF (10 mL) along with Int. 3 (1.36 g, 1.8 mL, 1.2 Eq, 12.03 mmol), HATU (4.5 g, 1.2 Eq, 12.03 mmol), and DIPEA (4.53 g, 6.11 mL, 3.5 Eq, 35.08 mmol). The pH was checked by indicator paper and DIPEA was added until the pH was confirmed to be basic. The reaction was stirred for 1 hour, diluted with water, and extracted with EtOAc (3×100 mL). The combined organics were washed with brine, dried over MgSO₄, filtered, and concentrated by rotary evaporation. The crude product was purified by silica gel chromatography (0 to 100% ethyl acetate in hexanes) to yield 249 (1.195 g, 2.52 mmol, 25% yield). LCMS (ESI+): m/z 473.2 (M+H⁺).

### Steps 2-3: (S)-3-(2-bromo-5-chlorophenyl)-2-(ethylamino)-N-(hex-5-en-1-yl)-N-methylpropanamide (250)

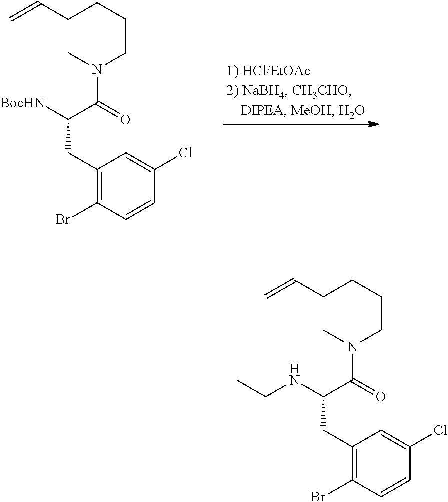 [SMILES: C=CCCCCN(C)C(=O)[C@H](Cc1cc(Cl)ccc1Br)NC(=O)OC(C)(C)C ; C=CCCCCN(C)C(=O)[C@H](Cc1cc(Cl)ccc1Br)NCC]

249 (1.195 g, 1 Eq, 2.522 mmol) was dissolved in 4M HCl/EtOAc and stirred for 1 hour. The reaction was diluted in saturated sodium bicarbonate EtOAc (3×30 mL). The combined organics were washed with brine, dried over MgSO₄, filtered, and concentrated by rotary evaporation. The residue was carried on without further purification. LCMS (ESI+): m/z 373.5 (M+H⁺).

The residue in was dissolved in MeOH (20 mL) and Water (2.0 mL). To this solution was added DIPEA (2.1 g, 2.8 mL, 6.4 Eq, 16 mmol), followed by a solution of acetaldehyde (433 mg, 550 μL, 3.90 Eq, 9.84 mmol) in ethanol. Sodium borohydride (400 mg, 4.19 Eq, 10.6 mmol) was added and the reaction was stirred overnight. Additional DIPEA (2.1 g, 2.8 mL, 6.4 Eq, 16 mmol), solution of acetaldehyde (433 mg, 550 μL, 3.90 Eq, 9.84 mmol) in ethanol, and sodium borohydride (400 mg, 4.19 Eq, 10.6 mmol) were added and the reaction was stirred for 2 hr. The reaction was concentrated by rotary evaporation and the crude product, 250 (1.01 g, 2.522 mmol, quant.), was used without further purification. LCMS (ESI+): m/z 401.2 (M+H⁺).

### Step 4: tert-butyl ((S)-1-(((S)-3-(2-bromo-5-chlorophenyl)-1-(hex-5-en-1-yl(methyl)amino)-1-oxopropan-2-yl)(ethyl)amino)-4-methyl-1-oxopentan-2-yl)carbamate (251)

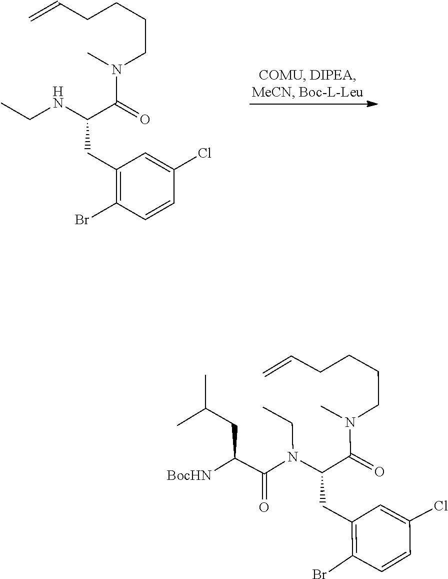 [SMILES: C=CCCCCN(C)C(=O)[C@H](Cc1cc(Cl)ccc1Br)NCC ; C=CCCCCN(C)C(=O)[C@H](Cc1cc(Cl)ccc1Br)N(CC)C(=O)[C@H](CC(C)C)NC(=O)OC(C)(C)C]

250 (1.01 g, 1 Eq, 2.51 mmol) was dissolved in MeCN (25 mL) along with COMU (8 g, 7.4 Eq, 19 mmol), Boc-L-Leucine monohydrate (4.5 g, 7.2 Eq, 18 mmol), and DIPEA (5.6 g, 7.5 mL, 17 Eq, 43 mmol). The reaction was refluxed at 85° C. for 24 hours and allowed to cool to room temperature before being concentrated by rotary evaporation. The residue was partitioned between water (20 mL) and EtOAc (20 mL), and the aqueous layer was extracted with EtOAc (3×20 mL). The combined organics were washed with brine, dried over MgSO₄, filtered, and concentrated by rotary evaporation. The crude product was purified by silica gel chromatography (0 to 100% EtOAc in hexanes) to yield 251 (440 mg, 2.51 mmol, 25%). LCMS (ESI+): m/z 617.8 (M+H⁺).

### Steps 5-9: tert-butyl ((S)-2-(((3S,6S,9S)-3-(2-bromo-5-chlorobenzyl)-6-isobutyl-1,4-dimethyl-2,5,8-trioxo-1,4,7-triazacyclohexadec-11-en-9-yl)(methyl)amino)-1-cyclopropyl-2-oxoethyl)carbamate (252)

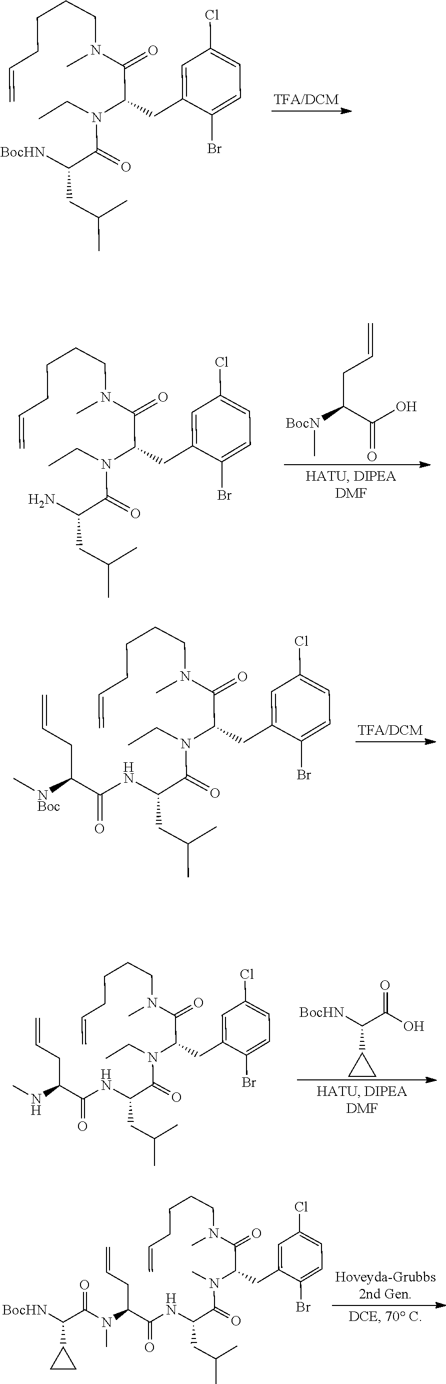

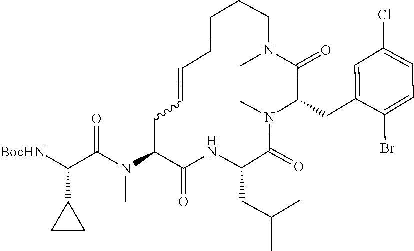 [SMILES: CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(Cl)ccc2Br)N(C)C1=O]

Steps 5-9 for the synthesis of 252 were carried out using reaction sequences and procedures outlined in Method A, Steps 4-8. LCMS (ESI): m/z 780.4 (M+H⁺).

### 2. Final Synthesis Procedures to Prepare Compounds of Formula (I)

a. Method 1

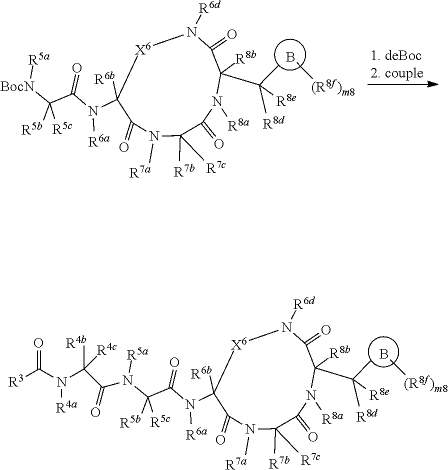

### Representative Procedure for Method 1: Example 118

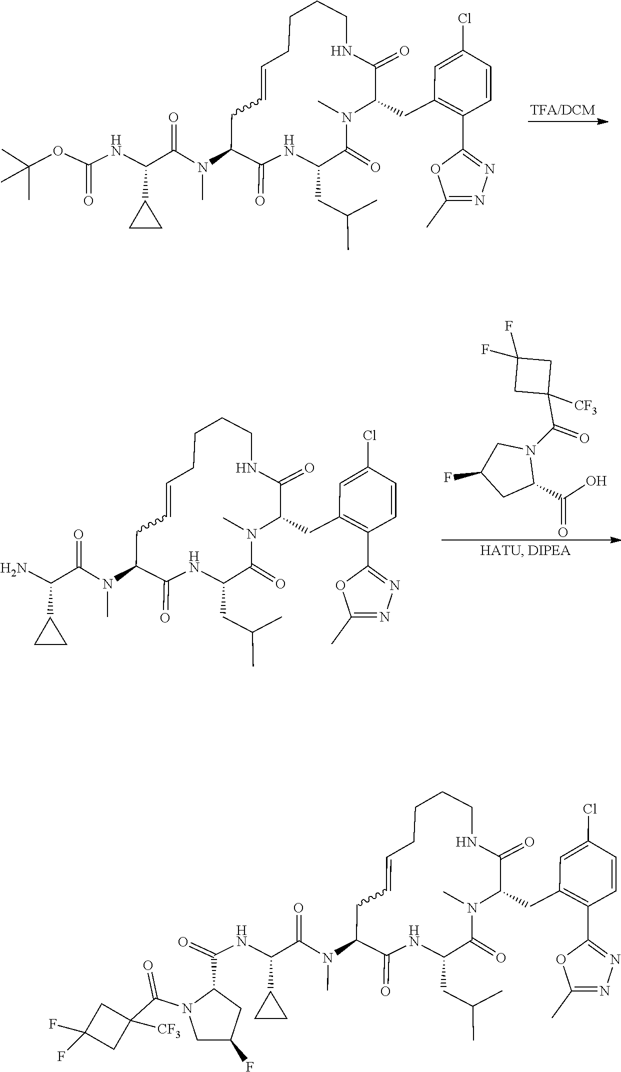 [SMILES: Cc1nnc(-c2ccc(Cl)cc2C[C@H]2C(=O)NCCCCC=CC[C@H](N(C)C(=O)[C@@H](NC(=O)OC(C)(C)C)C3CC3)C(=O)N[C@@H](CC(C)C)C(=O)N2C)o1 ; Cc1nnc(-c2ccc(Cl)cc2C[C@H]2C(=O)NCCCCC=CC[C@H](N(C)C(=O)[C@@H](N)C3CC3)C(=O)N[C@@H](CC(C)C)C(=O)N2C)o1 ; O=C(O)[C@@H]1C[C@@H](F)CN1C(=O)C1(C(F)(F)F)CC(F)(F)C1 ; Cc1nnc(-c2ccc(Cl)cc2C[C@H]2C(=O)NCCCCC=CC[C@H](N(C)C(=O)[C@@H](NC(=O)[C@@H]3C[C@@H](F)CN3C(=O)C3(C(F)(F)F)CC(F)(F)C3)C3CC3)C(=O)N[C@@H](CC(C)C)C(=O)N2C)o1]

### Representative Experimental for Method 1: Example 118

### Tert-butyl ((S)-2-(((3S,6S,9S)-3-(5-chloro-2-(5-methyl-1,3,4-oxadiazol-2-yl)benzyl)-6-isobutyl-4-methyl-2,5,8-trioxo-1,4,7-triazacyclohexadec-11-en-9-yl)(methyl)amino)-1-cyclopropyl-2-oxoethyl)carbamate (219)

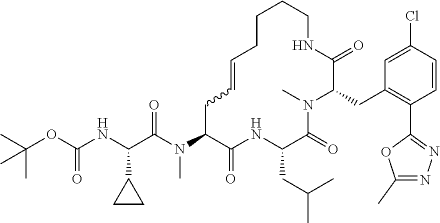 [SMILES: Cc1nnc(-c2ccc(Cl)cc2C[C@H]2C(=O)NCCCCC=CC[C@H](N(C)C(=O)[C@@H](NC(=O)OC(C)(C)C)C3CC3)C(=O)N[C@@H](CC(C)C)C(=O)N2C)o1]

The macrocyclic starting material for Method 1 was synthesized Using Method A with Int. 1, and 8. It was used as a mixture of E/Z isomers. LCMS (ESI+): m/z 770.4 (M+H).

### Step 1: (S)-2-amino-N-((3S,6S,9S)-3-(5-chloro-2-(5-methyl-1,3,4-oxadiazol-2-yl)benzyl)-6-isobutyl-4-methyl-2,5,8-trioxo-1,4,7-triazacyclohexadec-11-en-9-yl)-2-cyclopropyl-N-methylacetamide (220)

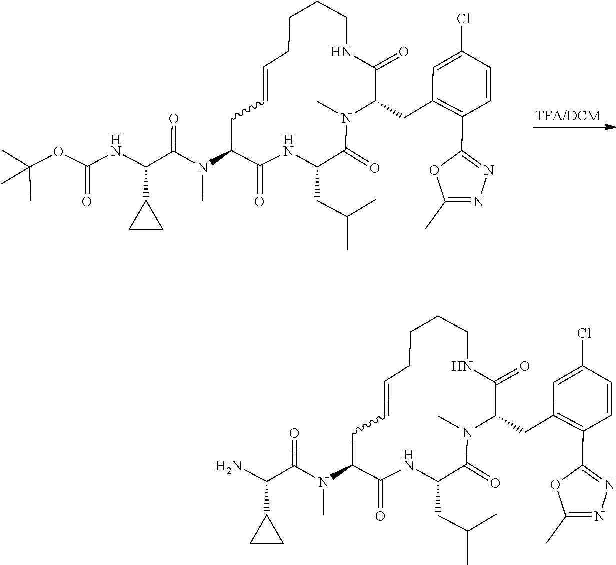 [SMILES: Cc1nnc(-c2ccc(Cl)cc2C[C@H]2C(=O)NCCCCC=CC[C@H](N(C)C(=O)[C@@H](NC(=O)OC(C)(C)C)C3CC3)C(=O)N[C@@H](CC(C)C)C(=O)N2C)o1 ; Cc1nnc(-c2ccc(Cl)cc2C[C@H]2C(=O)NCCCCC=CC[C@H](N(C)C(=O)[C@@H](N)C3CC3)C(=O)N[C@@H](CC(C)C)C(=O)N2C)o1]

219 (112 mg, 1 Eq, 145 μmol) was dissolved in DCM/TFA (3:1) and stirred until the deprotection was complete (monitored via LCMS). The volatile solvent was completely removed to afford a quantitative yield of 220 (97.4 mg, 145 μmol, 100%) as the TFA salt. LCMS (ESI+): m/z 732.4 (M+H).

### Step 2: (2S,4R)—N—((S)-2-(((3S,6S,9S)-3-(5-chloro-2-(5-methyl-1,3,4-oxadiazol-2-yl)benzyl)-6-isobutyl-4-methyl-2,5,8-trioxo-1,4,7-triazacyclohexadec-11-en-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 118)

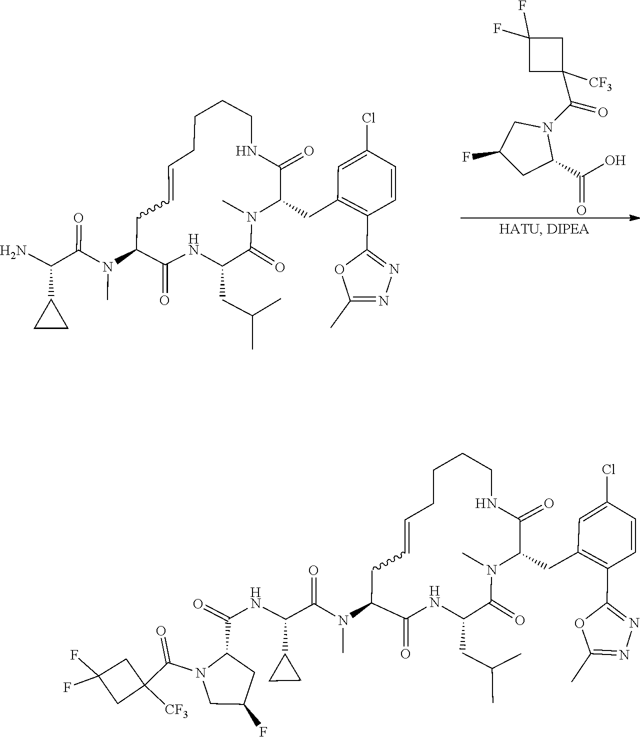 [SMILES: Cc1nnc(-c2ccc(Cl)cc2C[C@H]2C(=O)NCCCCC=CC[C@H](N(C)C(=O)[C@@H](N)C3CC3)C(=O)N[C@@H](CC(C)C)C(=O)N2C)o1 ; O=C(O)[C@@H]1C[C@@H](F)CN1C(=O)C1(C(F)(F)F)CC(F)(F)C1 ; Cc1nnc(-c2ccc(Cl)cc2C[C@H]2C(=O)NCCCCC=CC[C@H](N(C)C(=O)[C@@H](NC(=O)[C@@H]3C[C@@H](F)CN3C(=O)C3(C(F)(F)F)CC(F)(F)C3)C3CC3)C(=O)N[C@@H](CC(C)C)C(=O)N2C)o1]

220 (48.7 mg, 1 Eq, 72.7 μmol) and Int. 141 (27.8 mg, 1.2 Eq, 87.2 μmol) were dissolved in DMF (2.0 mL). DIPEA (56.3 mg, 75.9 μL, 6 equiv., 436 μmol) and HATU (33.2 mg, 1.2 Eq, 87.2 μmol) were added sequentially and the reaction was continued until the consumption of starting material (monitored via LCMS). The reaction mixture was neutralized to pH ˜7 with 1N HCl and directly purified by RP-HPLC (C18, 40% to 75% MeCN/H₂O with 0.5% formic acid). Example 118 was isolated as two peaks. The first eluting (28.5 mg, 29.3 μmol, 40.4%) and the second eluting peak (18.2 mg, 18.7 μmol, 25.7%) were isolated as a white solid after lyophilization. Stereochemistry of isolated peaks was not assigned. LCMS data was identical for both peaks. LCMS (ESI+): m/z 971.3 (M+H).

b. Method 2

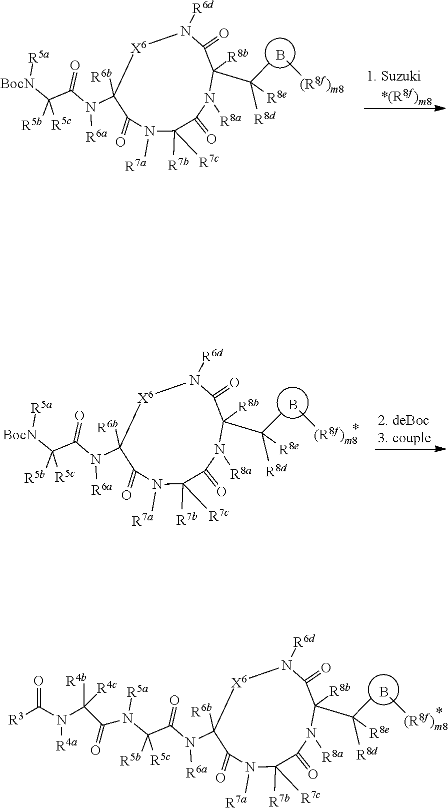

### Representative Procedure for Method 2: Example 273

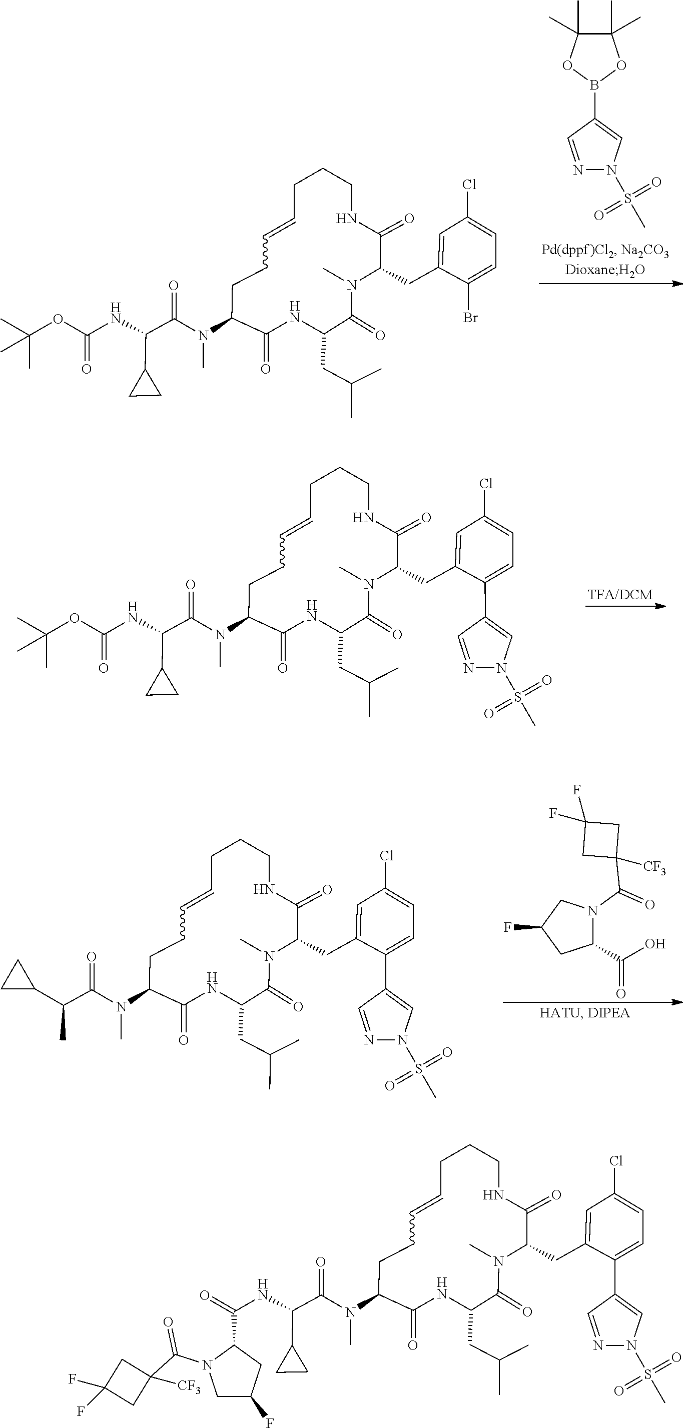

### Representative Experimental for Method 2: Example 273

### Tert-butyl ((S)-2-(((3S,6S,9S)-3-(2-bromo-5-chlorobenzyl)-6-isobutyl-4-methyl-2,5,8-trioxo-1,4,7-triazacyclohexadec-12-en-9-yl)(methyl)amino)-1-cyclopropyl-2-oxoethyl)carbamate (221)

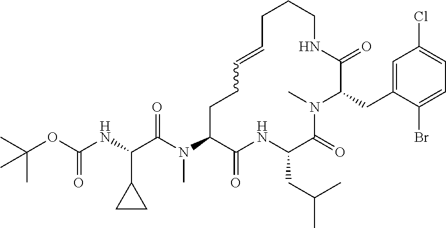 [SMILES: CC(C)C[C@@H]1NC(=O)[C@@H](N(C)C(=O)[C@@H](NC(=O)OC(C)(C)C)C2CC2)CCC=CCCCNC(=O)[C@H](Cc2cc(Cl)ccc2Br)N(C)C1=O]

The macrocyclic Starting material for Method 2 was synthesized Using Method A with Int. 2, 30, and 42. It was used as a mixture of E/Z isomers. LCMS (ESI+): m/z 766.2 (M+H).

### Step 1: tert-butyl ((S)-2-(((3S,6S,9S)-3-(5-chloro-2-(1-(methylsulfonyl)-1H-pyrazol-4-yl)benzyl)-6-isobutyl-4-methyl-2,5,8-trioxo-1,4,7-triazacyclohexadec-12-en-9-yl)(methyl)amino)-1-cyclopropyl-2-oxoethyl)carbamate (222)

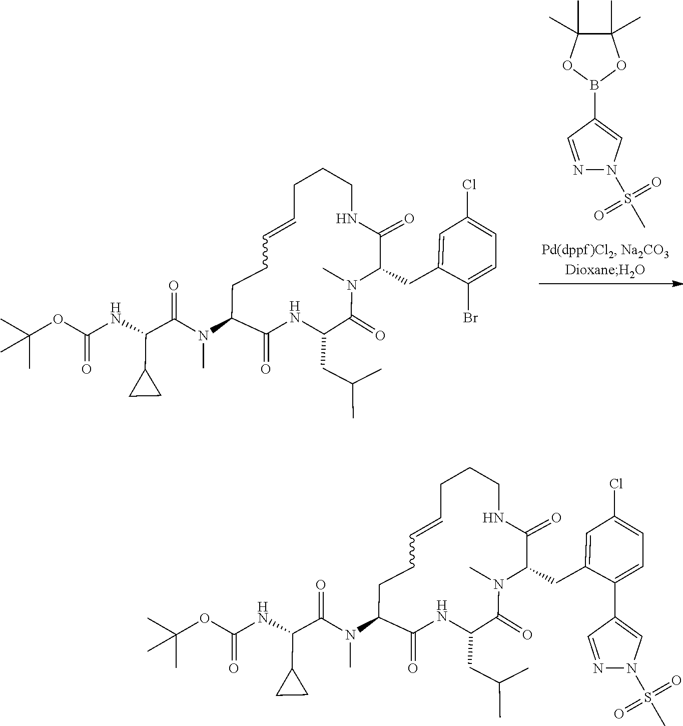 [SMILES: CC(C)C[C@@H]1NC(=O)[C@@H](N(C)C(=O)[C@@H](NC(=O)OC(C)(C)C)C2CC2)CCC=CCCCNC(=O)[C@H](Cc2cc(Cl)ccc2Br)N(C)C1=O ; CC1(C)OB(c2cnn(S(C)(=O)=O)c2)OC1(C)C ; CC(C)C[C@@H]1NC(=O)[C@@H](N(C)C(=O)[C@@H](NC(=O)OC(C)(C)C)C2CC2)CCC=CCCCNC(=O)[C@H](Cc2cc(Cl)ccc2-c2cnn(S(C)(=O)=O)c2)N(C)C1=O]

221 (124 mg, 2 Eq, 456 μmol), PdCl2(dppf)-DCM adduct (186 mg, 1 Eq, 228 mol), Int. 64, and sodium carbonate (96.7 mg, 4 equiv., 912 μmol) were dissolved in 4:1 Dioxane/Water (6.3 mL). The reaction was degassed for 10 min with Ar and then heated to 80° C. until the complete consumption of starting material monitored by LCMS. The vial was then cooled and adjusted to pH ˜7 with 1N HCl. The solution was filtered and directly purified by RP-HPLC (C18, 50% to 80% MeCN/H₂O with 0.5% formic acid) to obtain 222 (150 mg, 180 μmol, 79%) as a white solid after lyophilization. LCMS (ESI+): m/z 832.4 (M+H).

### Step 2: (S)-2-amino-N-((3S,6S,9S)-3-(5-chloro-2-(1-(methylsulfonyl)-1H-pyrazol-4-yl)benzyl)-6-isobutyl-4-methyl-2,5,8-trioxo-1,4,7-triazacyclohexadec-12-en-9-yl)-2-cyclopropyl-N-methylacetamide (223)

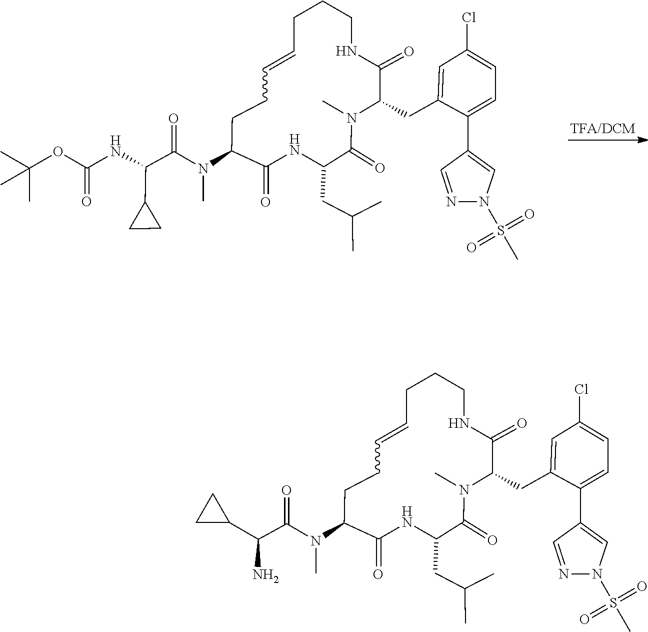 [SMILES: CC(C)C[C@@H]1NC(=O)[C@@H](N(C)C(=O)[C@@H](NC(=O)OC(C)(C)C)C2CC2)CCC=CCCCNC(=O)[C@H](Cc2cc(Cl)ccc2-c2cnn(S(C)(=O)=O)c2)N(C)C1=O ; CC(C)C[C@@H]1NC(=O)[C@@H](N(C)C(=O)[C@@H](N)C2CC2)CCC=CCCCNC(=O)[C@H](Cc2cc(Cl)ccc2-c2cnn(S(C)(=O)=O)c2)N(C)C1=O]

222 (126.6 mg, 1 Eq, 152.1 μmol) was dissolved in DCM/TFA (3:1) and stirred until the deprotection was complete (monitored via LCMS). The volatile solvent was completely removed to afford a quantitative yield of 223 (111.4 mg, 152.1 μmol, 100.0%) as the TFA salt. LCMS (ESI+): m/z 732.4 (M+H).

### Step 3: (2S,4R)—N—((S)-2-(((3S,6S,9S)-3-(5-chloro-2-(1-(methylsulfonyl)-1H-pyrazol-4-yl)benzyl)-6-isobutyl-4-methyl-2,5,8-trioxo-1,4,7-triazacyclohexadec-12-en-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 273)

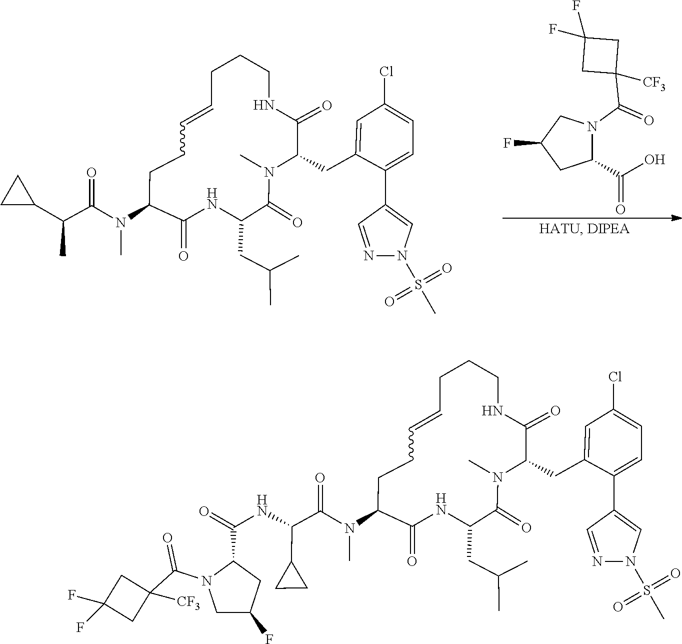 [SMILES: CC(C)C[C@@H]1NC(=O)[C@@H](N(C)C(=O)[C@@H](C)C2CC2)CCC=CCCCNC(=O)[C@H](Cc2cc(Cl)ccc2-c2cnn(S(C)(=O)=O)c2)N(C)C1=O ; O=C(O)[C@@H]1C[C@@H](F)CN1C(=O)C1(C(F)(F)F)CC(F)(F)C1 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCC=CCCCNC(=O)[C@H](Cc2cc(Cl)ccc2-c2cnn(S(C)(=O)=O)c2)N(C)C1=O]

223 as the TFA salt (111.4 mg, 1 Eq, 152.1 μmol) and Int. 141 (58.27 mg, 1.2 Eq, 182.5 μmol) were dissolved in DMF (5.0 mL). DIPEA (118.0 mg, 159 μL, 6 Eq, 912.7 μmol) and HATU (69.41 mg, 1.2 Eq, 182.5 μmol) was added sequentially and the reaction was continued until consumption of starting material (monitored by LCMS). The reaction mixture was neutralized to pH ˜7 with 1N HCl and directly purified by RP-HPLC (C18, 40% to 70% MeCN/H₂O with 0.5% formic acid). Example 273 (31.72 mg, 30.69 μmol, 20.18%) was obtained as a white solid after lyophilization. LCMS (ESI+): m/z 1033.4 (M+H).

c. Method 3

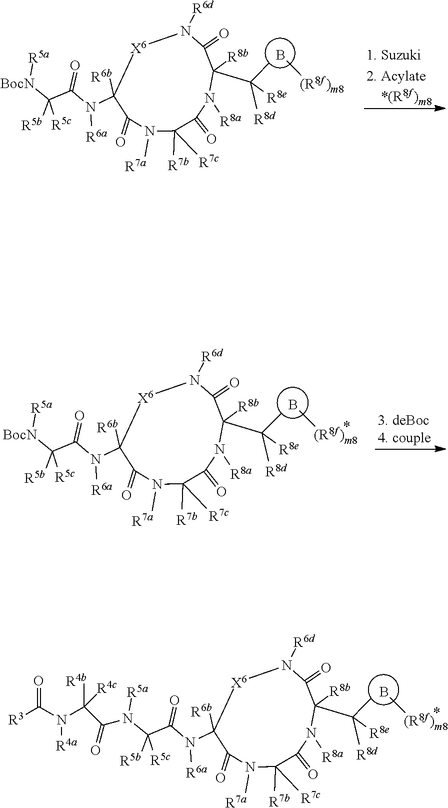

### Representative Procedure for Method 3: Example 53

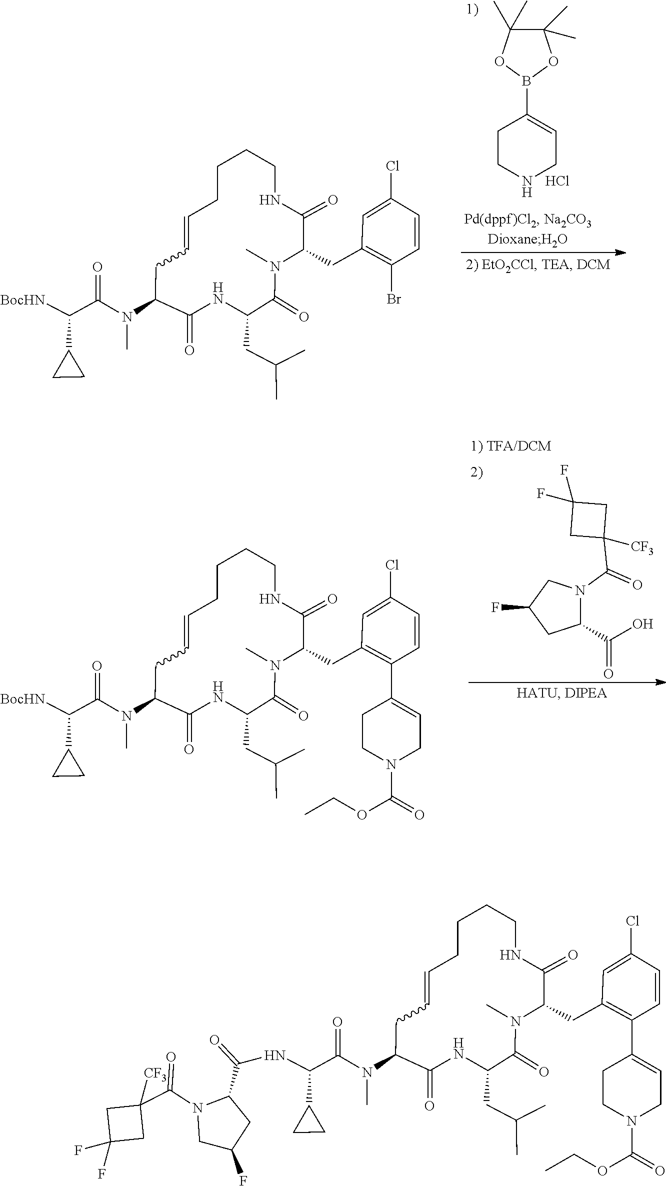

### Representative Experimental for Method 3: Example 53

### Tert-butyl ((S)-2-(((3S,6S,16S)-16-(2-bromo-5-chlorobenzyl)-3-isobutyl-1-methyl-2,5,15-trioxo-113-chlora-4,14-diazacyclohexadec-8-en-6-yl)(methyl)amino)-1-cyclopropyl-2-oxoethyl)carbamate (224)

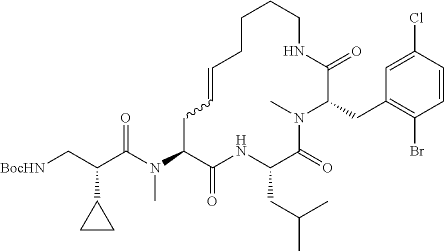 [SMILES: CC(C)C[C@@H]1NC(=O)[C@@H](N(C)C(=O)[C@@H](CNC(=O)OC(C)(C)C)C2CC2)CC=CCCCCNC(=O)[C@H](Cc2cc(Cl)ccc2Br)N(C)C1=O]

The starting material for Method 3, 224, was synthesized according to method A using Int. 1 and 30. LCMS (ESI+): 766.2 m/z (M+H).

### Steps 1-2: Ethyl 4-(2-(((3S,6S,9S)-9-((S)-2-((tert-butoxycarbonyl)amino)-2-cyclopropyl-N-methylacetamido)-6-isobutyl-4-methyl-2,5,8-trioxo-1,4,7-triazacyclohexadec-11-en-3-yl)methyl)-4-chlorophenyl)-3,6-dihydropyridine-1 (2H)-carboxylat (225)

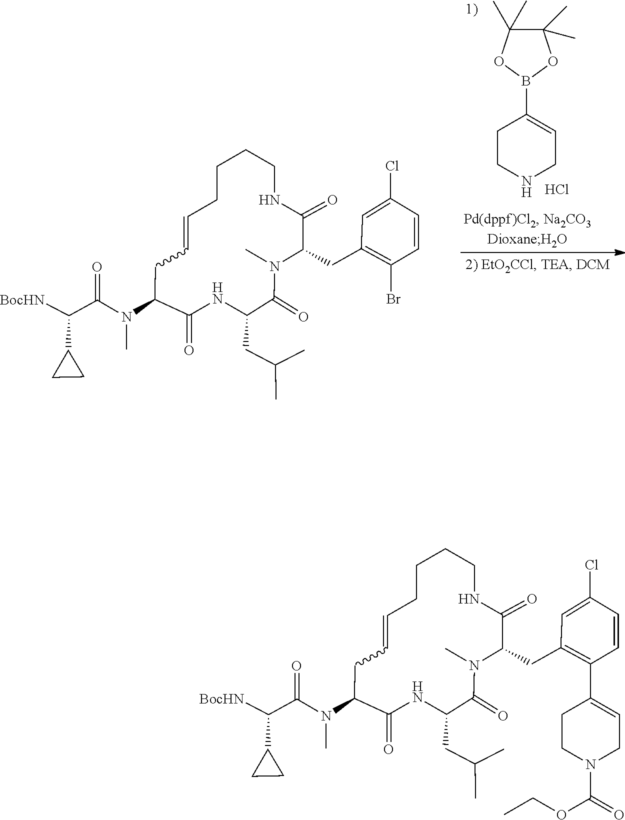

224 (100 mg, 1 Eq, 127 μmol), Int. 67 (31.1 mg, 1 Eq, 127 μmol), Na₂CO₃ (40.3 mg, 3 Eq, 380 μmol) and PdCl₂(dppf) (10.4 mg, 0.1 Eq, 12.7 μmol) were dissolved in 4:1 Dioxane/Water (4 mL). The reaction was degassed for 10 min with Ar and then heated to 80° C. The reaction was stopped by the addition of 5 mL water upon the consumption of starting material (monitored by LCMS). The aqueous layer was extracted EtOAc (2×10 mL). The combined organic layer was washed with brine, dried over MgSO₄, filtered, and concentrated. The material could either be purified by RP-HPLC (C18, 10% to 50% MeCN/H₂O with 0.5% formic acid) or used directly in the next reaction.

The crude reaction product was dissolved in DCM (2 mL), cooled in an ice bath before the addition of TEA (64.2 mg, 88.4 μL, 5 Eq, 634 μmol). ethyl chloroformate (20.6 mg, 18.3 μL, 1.5 Eq, 190 μmol) was added slowly to the cooled solution. The reaction was stirred at 0° C. until the complete consumption of starting material. Once complete the volatile solvents were completely removed and the crude residue was purified directly by RP-HPLC (C18, 50% to 80% MeCN/H₂O with 0.5% formic acid) which afforded 225 (54 mg, 64 μmol, 51%) as an off-white solid. LCMS (ESI+): 841.3 m/z (M+H).

### Steps 3-4: Ethyl 4-(4-chloro-2-(((3S,6S,9S)-9-((S)-2-cyclopropyl-2-((2S,4R)-1-(3,3-difluoro-1-(trifluoromethyl)cyclobutane-1-carbonyl)-4-fluoropyrrolidine-2-carboxamido)-N-methylacetamido)-6-isobutyl-4-methyl-2,5,8-trioxo-1,4,7-triazacyclohexadec-11-en-3-yl)methyl)phenyl)-3,6-dihydropyridine-1 (2H)-carboxylate (Example 53)

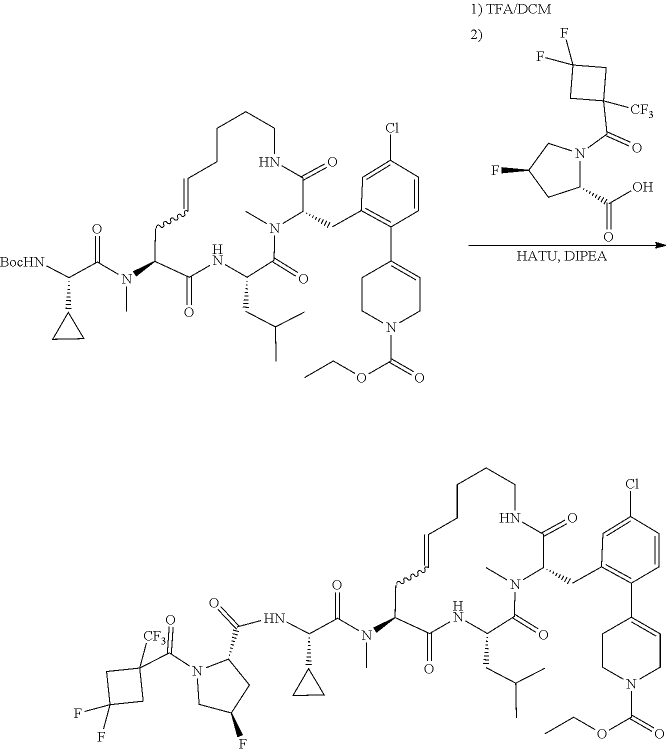 [SMILES: CCOC(=O)N1CC=C(c2ccc(Cl)cc2C[C@H]2C(=O)NCCCCC=CC[C@H](N(C)C(=O)[C@@H](NC(=O)OC(C)(C)C)C3CC3)C(=O)N[C@@H](CC(C)C)C(=O)N2C)CC1 ; O=C(O)[C@@H]1C[C@@H](F)CN1C(=O)C1(C(F)(F)F)CC(F)(F)C1 ; CCOC(=O)N1CC=C(c2ccc(Cl)cc2C[C@H]2C(=O)NCCCCC=CC[C@H](N(C)C(=O)[C@@H](NC(=O)[C@@H]3C[C@@H](F)CN3C(=O)C3(C(F)(F)F)CC(F)(F)C3)C3CC3)C(=O)N[C@@H](CC(C)C)C(=O)N2C)CC1]

225 (107 mg, 1 Eq, 127 μmol) was dissolved in DCM/TFA (3:1) and stirred until the deprotection was complete (ministered by LCMS). The volatile solvent was completely removed to afford a quantitative yield of the free amine.

The deprotected amine and Int. 141 (44.8 mg, 1.1 Eq, 140 μmol) were dissolved in DMF (1 mL). DIPEA (98.9 mg, 133 μL, 6 Eq, 765 μmol) and HATU (53.3 mg, 1.1 Eq, 140 mol) were added sequentially. and the reaction continued until the consumption of my starting material by LCMS. The reaction mixture was neutralized to pH ˜7 with 1 N HCl and directly purified by RP-HPLC(C18, 40% to 70% MeCN/H₂O with 0.5% formic acid). Example 53 (48.8 mg, 46.8 μmol, 37%) was obtained as a white solid after lyophilization. LCMS (ESI+): m/z 1043.6 (M+H).

d. Method 4

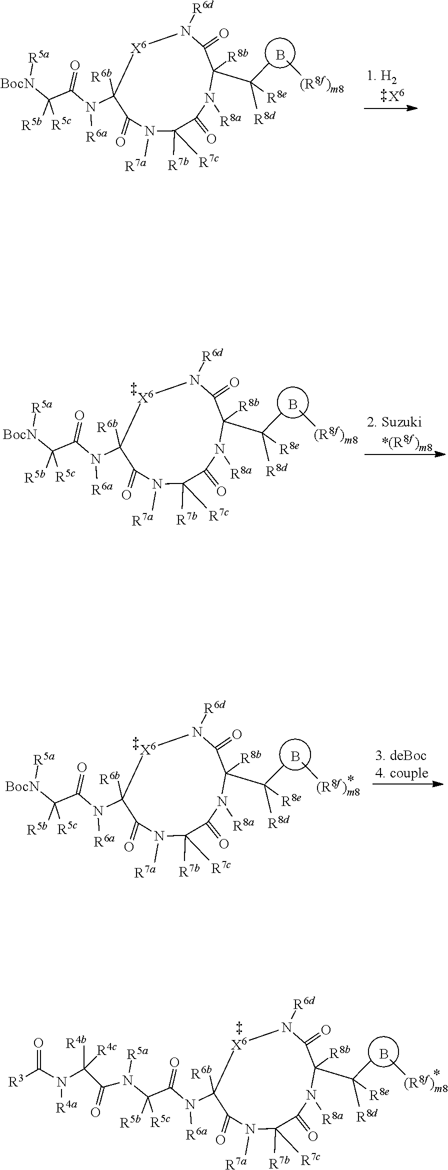

### Representative Procedure for Method 4: Example 50

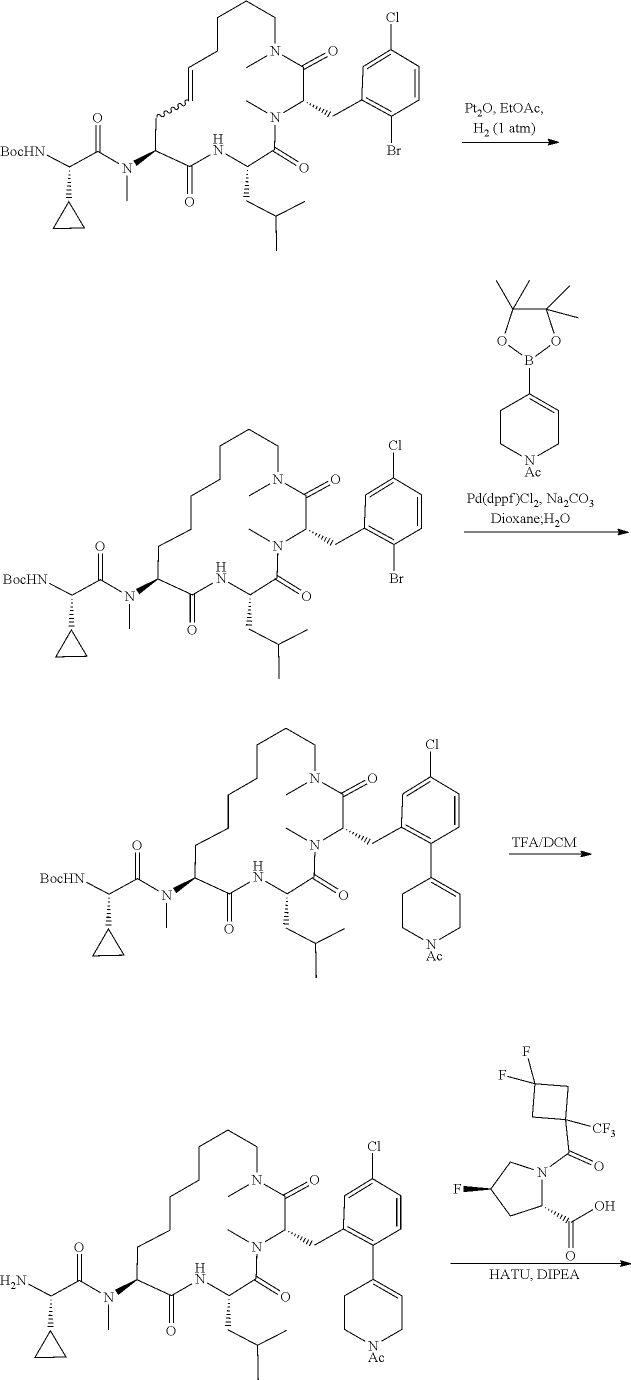

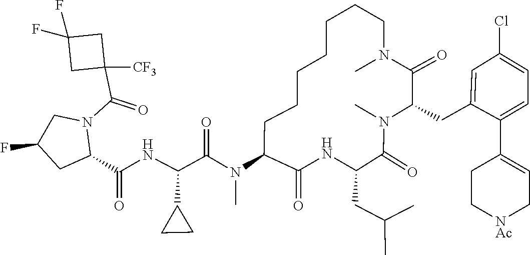

### Representative Experimental for Method 4: Example 50

### Tert-butyl ((S)-2-(((3S,6S,9S)-3-(2-bromo-5-chlorobenzyl)-6-isobutyl-1,4-dimethyl-2,5,8-trioxo-1,4,7-triazacyclohexadec-11-en-9-yl)(methyl)amino)-1-cyclopropyl-2-oxoethyl)carbamate (226)

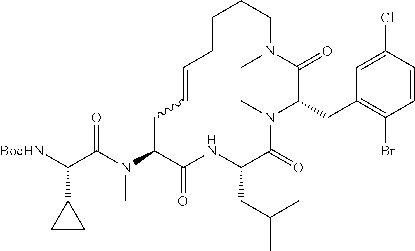 [SMILES: CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(Cl)ccc2Br)N(C)C1=O]

The macrocycle starting material for method 4 (226), was synthesized using Method A with Int. 30. It was used as a mixture of E/Z isomers (˜9:1) LCMS: m/z [M+H]. LCMS (ESI+): 780.3 m/z (M+H).

### Step 1: ((S)-2-(((3S,6S,9S)-3-(2-bromo-5-chlorobenzyl)-6-isobutyl-4-methyl-2,5,8-trioxo-1,4,7-triazacyclohexadecan-9-yl)(methyl)amino)-1-cyclopropyl-2-oxoethyl)carbamate (227)

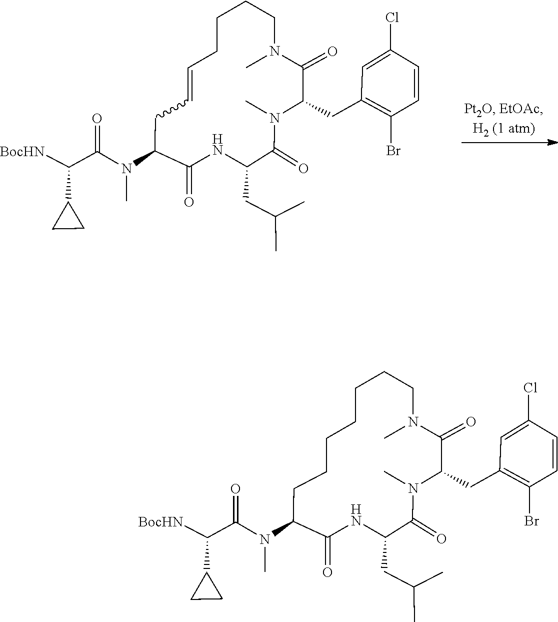 [SMILES: CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(Cl)ccc2Br)N(C)C1=O ; CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2Br)N(C)C1=O]

226 (12.0 g, 1 Eq, 15.6 mmol) was dissolved in EtOAc (80 ml) by stirring at ambient temperature. platinum(IV) oxide (284 mg, 0.08 Eq, 1.25 mmol) was added to the stirring reaction mixture. The reaction was then placed under an atmosphere of hydrogen and stirred vigorously until LCMS analysis showed complete consumption of starting material. The reaction mixture was filtered over a pad of Celite. The Filter was washed with EtOAc (2×50 mL). The organic solvent was concentrated to afford 227 (11.8 g, 15.3 mmol, 98%) as an off white solid. The reaction was used without further purification. LCMS: m/z 782.2 [M+H]⁺

### Step 2: tert-butyl ((S)-2-(((3S,6S,9S)-3-(2-(1-acetyl-1,2,3,6-tetrahydropyridin-4-yl)-5-chlorobenzyl)-6-isobutyl-1,4-dimethyl-2,5,8-trioxo-1,4,7-triazacyclohexadecan-9-yl)(methyl)amino)-1-cyclopropyl-2-oxoethyl)carbamate (228)

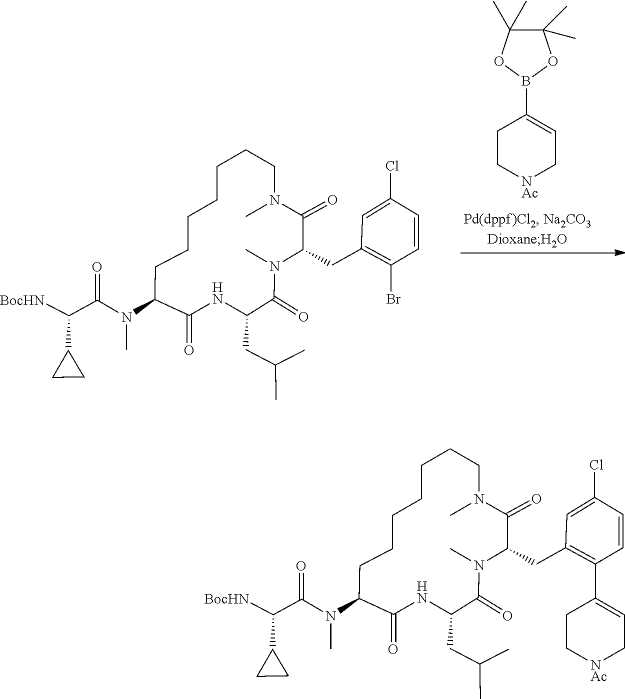

227 (34.5 mg, 1.0 Eq, 44.0 μmol), Na₂CO₃ (14.0 mg, 3 Eq, 132 μmol), Int. 47 (22.1 mg, 2.0 Eq, 88.1 μmol) and PdCl₂(dppf)-CH₂Cl₂ adduct (3.60 mg, 0.1 Eq, 4.40 μmol) were dissolved in 4:1 Dioxane/Water (4 mL). The reaction was degassed for 10 min with Ar and then heated to 80° C. until the complete consumption of starting material. The vial was then cooled and adjusted to pH ˜7 with 1 N HCl. The homogeneous solution was directly purified by RP-HPLC (C18, 50% to 85% MeCN/H₂O with 0.5% formic acid) to obtain 228 (31.6 mg, 44.0 μmol, 86.7%) as a white solid after lyophilization. LCMS (ESI+): m/z 827.4 (M+H).

### Step 3: (S)—N-((3S,6S,9S)-3-(2-(1-acetyl-1,2,3,6-tetrahydropyridin-4-yl)-5-chlorobenzyl)-6-isobutyl-1,4-dimethyl-2,5,8-trioxo-1,4,7-triazacyclohexadecan-9-yl)-2-amino-2-cyclopropyl-N-methylacetamide TFA salt (229)

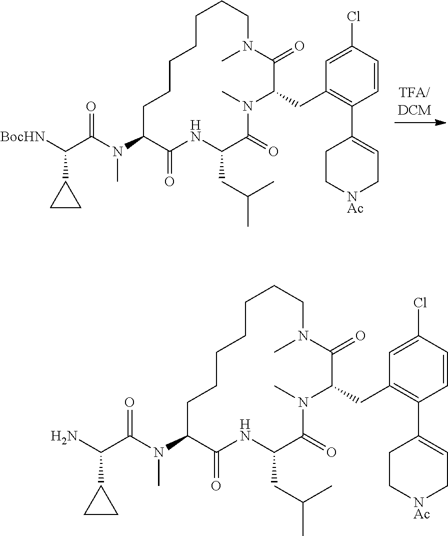

228 (31.6 mg, 1 Eq, 38.2 μmol) was dissolved in 6 mL DCM/TFA (3:1) and stirred until the deprotection was complete (monitored by LCMS). The volatile solvent was completely removed to afford a quantitative yield of 229. LCMS (ESI+): m/z 727.4 (M+H).

### Step 4: (2S,4R)—N—((S)-2-(((3S,6S,9S)-3-(2-(1-acetyl-1,2,3,6-tetrahydropyridin-4-yl)-5-chlorobenzyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 50)

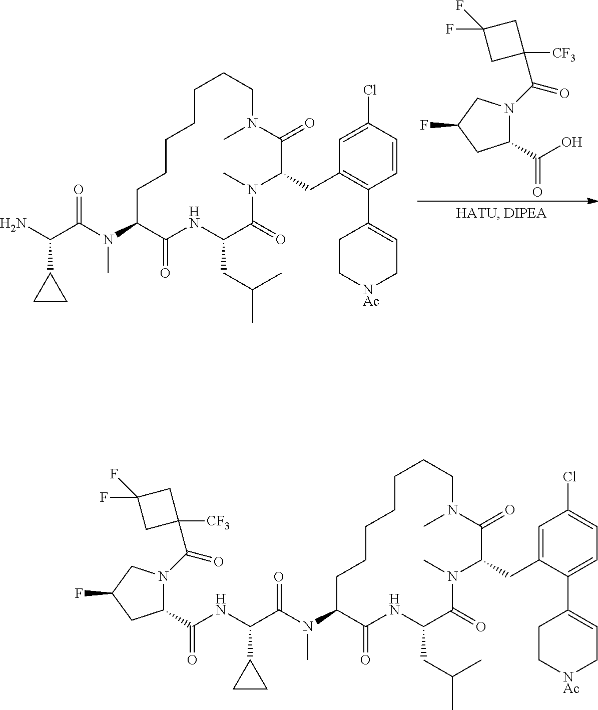

The deprotected amine and Int. 141 (13.4 mg, 1.1 Eq, 42.1 μmol) were dissolved in DMF (1 mL). DIPEA (29.7 mg, 40.0 μL, 6 Eq, 229 μmol) and HATU (16.0 mg, 1.1 Eq, 42.1 mol) were added sequentially and the reaction was continued until the consumption of starting material (monitored by LCMS). The reaction mixture was neutralized to pH ˜7 with 1N HCl and directly purified by RP-HPLC (C18, 45% to 75% MeCN/H₂O with 0.5% formic acid). Example 50 (25.0 mg, 24.3 μmol, 64%) was obtained as a white solid after lyophilization. LCMS (ESI+): m/z 1028.6.4 (M+H).

e. Method 5

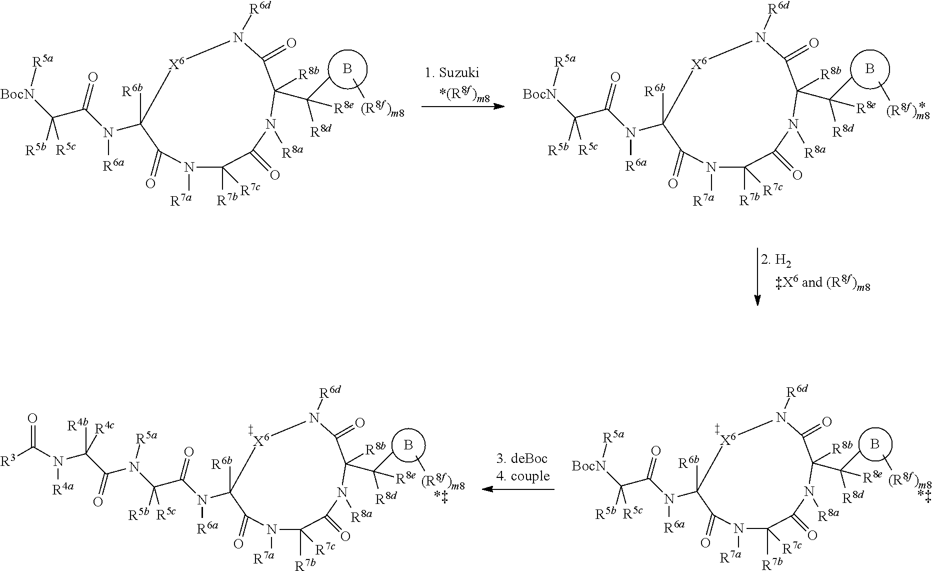

### Representative Procedure for Method 5: Example 45

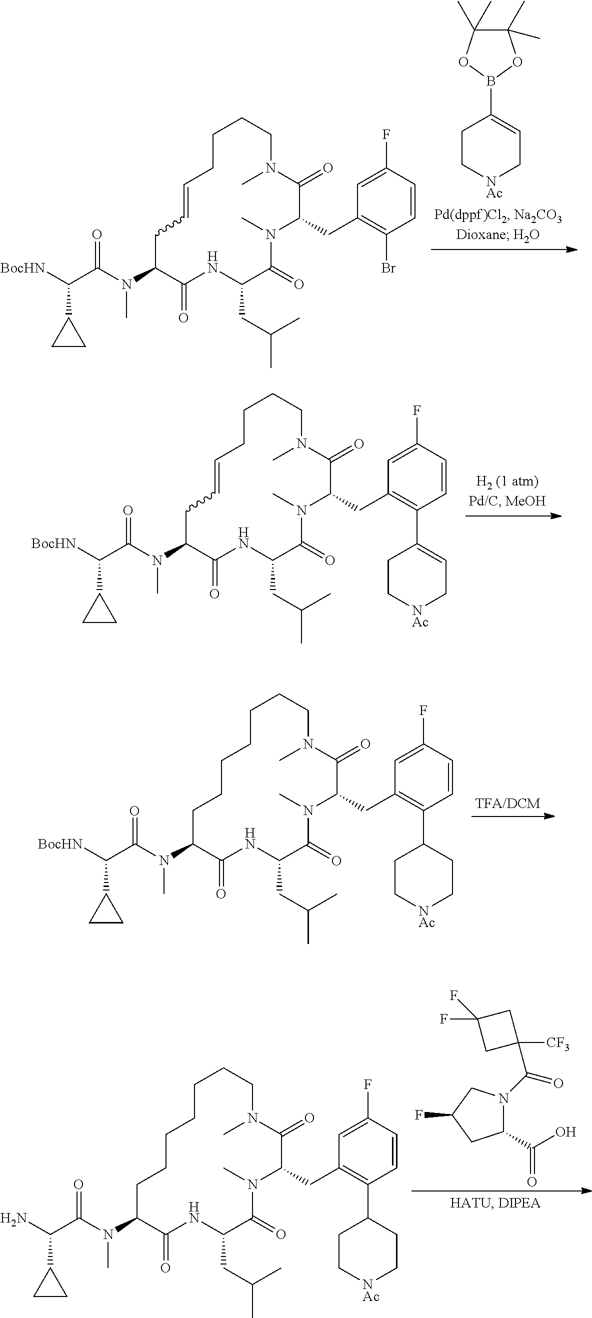

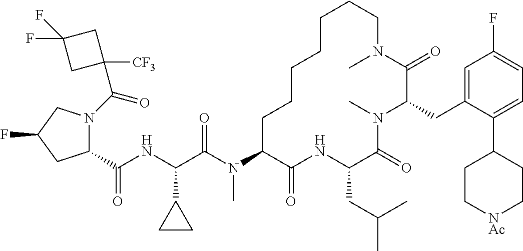

### Representative Experimental for Method 5: Example 45

### Step 1: Tert-butyl ((S)-2-(((3S,6S,9S)-3-(2-(1-acetyl-1,2,3,6-tetrahydropyridin-4-yl)-5-fluorobenzyl)-6-isobutyl-1,4-dimethyl-2,5,8-trioxo-1,4,7-triazacyclohexadec-11-en-9-yl)(methyl)amino)-1-cyclopropyl-2-oxoethyl)carbamate (230)

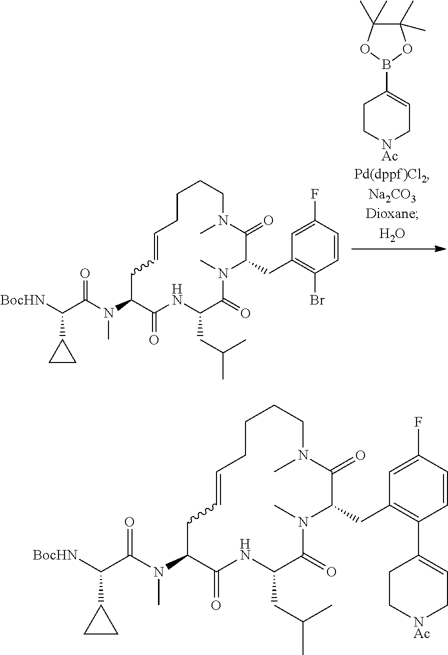

208 (300 mg, 1 Eq, 392 μmol), Int. 47 (128 mg, 1.3 Eq, 510 μmol), Na₂CO₃ (125 mg, 3 Eq, 1.18 mmol) and PdCl₂(dppf)-DCM adduct (32.0 mg, 0.1 Eq, 39.2 μmol) were dissolved in 4:1 Dioxane/Water (10 mL). The reaction was degassed for 10 min with Ar and then heated to 80° C. until the complete consumption of starting material monitored by LCMS. The vial was then cooled and adjusted to pH ˜7 with 1N HCl. The solution was concentrated to ˜4 mL, filtered, then directly purified by RP-HPLC (C18, 50% to 80% MeCN/H₂O with 0.5% formic acid) to obtain Int. 430 (200 mg, 247 μmol, 63%). LCMS: m/z 809.4 [M+H].

### Step 2: Tert-butyl ((S)-2-(((3S,6S,9S)-3-(2-(1-acetyl-1,2,3,6-tetrahydropyridin-4-yl)-5-fluorobenzyl)-6-isobutyl-1,4-dimethyl-2,5,8-trioxo-1,4,7-triazacyclohexadecan-9-yl)(methyl)amino)-1-cyclopropyl-2-oxoethyl)carbamate (231)

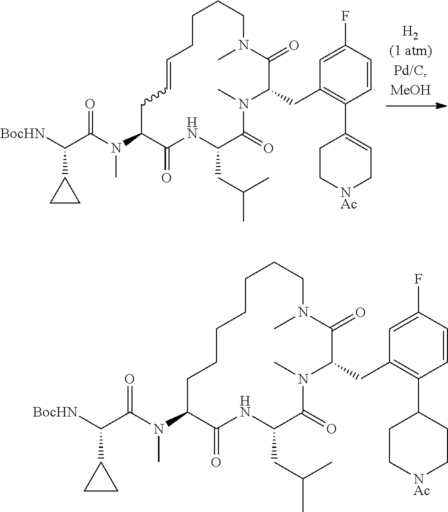

230 (100 mg, 1 Eq, 124 μmol) was dissolved in MeOH (2 mL). Pd/C (100 mg) was carefully added, and the reaction was stirred under an atmosphere of H₂. Upon the consumption of starting material (monitored by LCMS), the reaction was filtered over a pad of celite. The filter cake was washed with EtOAc (2×20 mL). the filtrate was concentrated to afford 231 (85.7 mg, 105 μmol, 85%) d as an off-white solid. It was used without any further purification. LCMS: m/z 813.5 [M+H].

### Step 3: (S)—N-((3S,6S,9S)-3-(2-(1-acetylpiperidin-4-yl)-5-fluorobenzyl)-6-isobutyl-1,4-dimethyl-2,5,8-trioxo-1,4,7-triazacyclohexadecan-9-yl)-2-amino-2-cyclopropyl-N-methylacetamide (232)

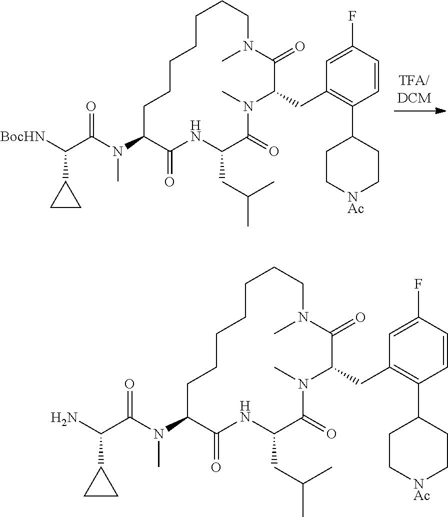

231 (40.0 mg, 1 Eq, 49.2 μmol) was dissolved in 2 ml DCM/TFA (3:1) and stirred until the deprotection was complete (monitored by LCMS). The volatile solvent was completely removed to afford a quantitative yield of 232 as the TFA salt. LCMS: m/z 713.4 [M+H].

### Step 4: (2S,4R)—N—((S)-2-(((3S,6S,9S)-3-(2-(1-acetylpiperidin-4-yl)-5-fluorobenzyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 45)

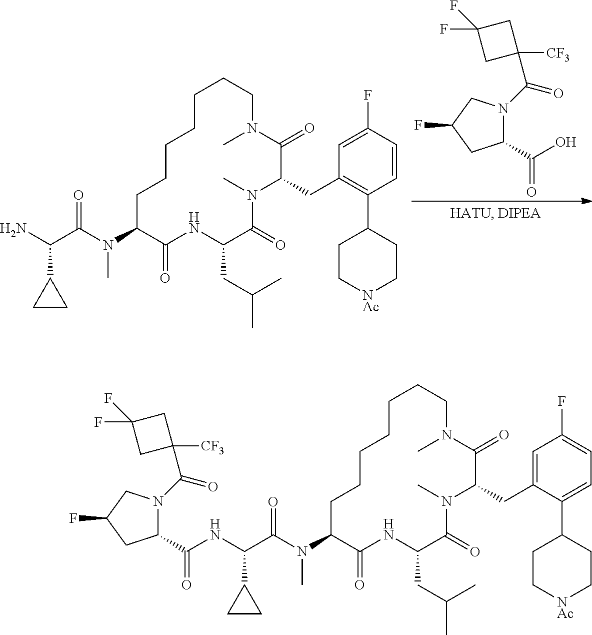

232 as the TFA salt (40.0 mg), Int. 141 (17.3 mg, 1.1 Eq, 54.1 μmol) and HATU (20.6 mg, 1.1 Eq, 54.1 μmol) were dissolved in DMF (2 mL). The reaction was stirred and DIPEA (38.2 mg, 51.4 μL, 6 Eq, 295 μmol) was added slowly. The reaction continued until the consumption of starting material (monitored by LCMS). The reaction mixture was neutralized to pH ˜7 with 1N HCl and directly purified by RP-HPLC (C18, 40% to 70% MeCN/H₂O with 0.5% formic acid). Example 45 (22.0 mg, 21.7 μmol, 44%) was isolated as a white solid upon lyophilization. LCMS: m/z 1015.5[M+H].

f. Method 6

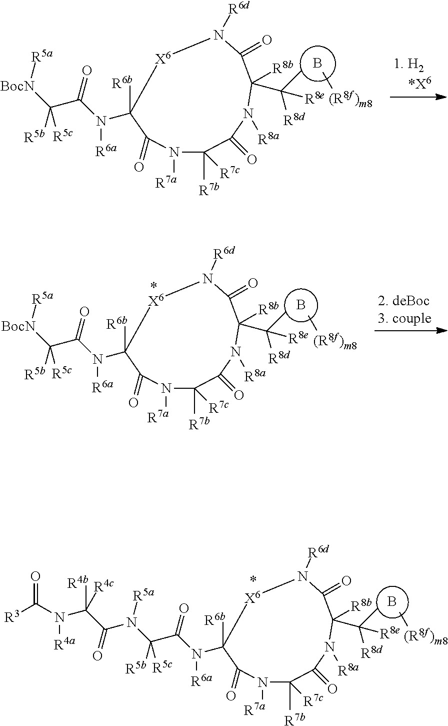

### Representative Procedure for Method 6: Example 331

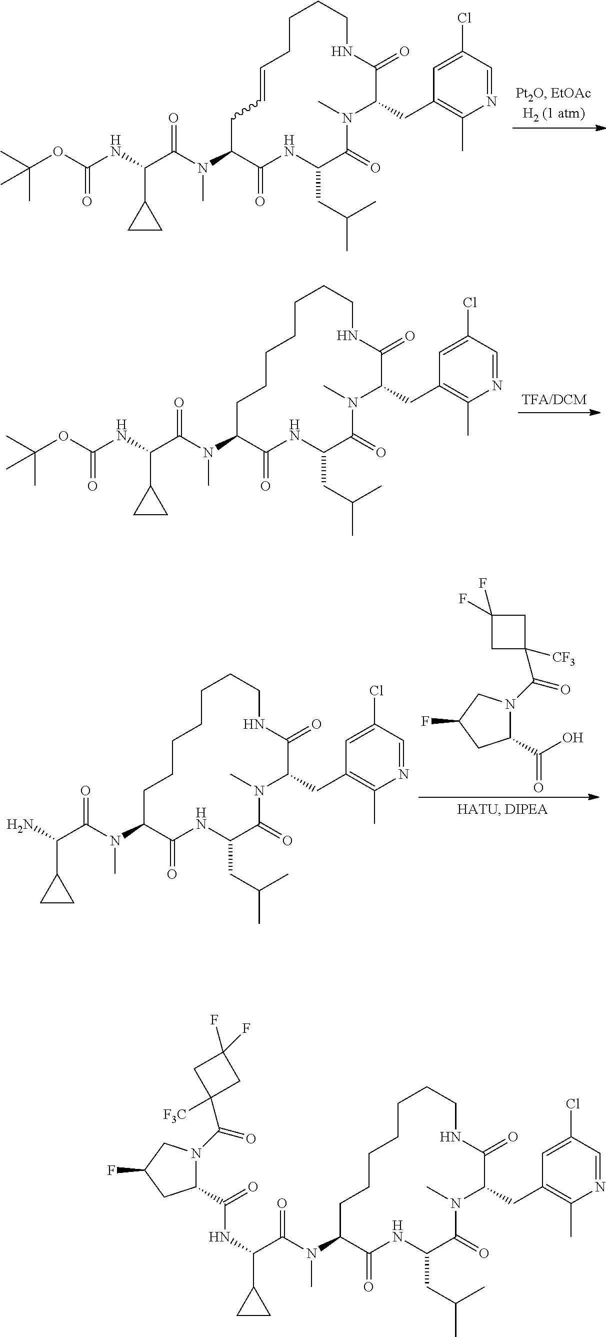

### Representative Experimental For Method 6: Example 331

### Tert-butyl ((S)-2-(((3S,6S,9S)-3-((5-chloro-2-methylpyridin-3-yl)methyl)-6-isobutyl-4-methyl-2,5,8-trioxo-1,4,7-triazacyclohexadec-11-en-9-yl)(methyl)amino)-1-cyclopropyl-2-oxoethyl)carbamate (233)

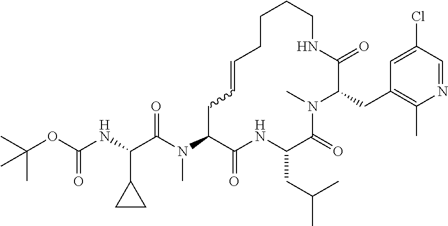 [SMILES: Cc1ncc(Cl)cc1C[C@H]1C(=O)NCCCCC=CC[C@H](N(C)C(=O)[C@@H](NC(=O)OC(C)(C)C)C2CC2)C(=O)N[C@@H](CC(C)C)C(=O)N1C]

The macrocycle starting material for Method 6, 233, was synthesized using Method A with Int. 1 and 27. It was used as a mixture of E/Z isomers. LCMS (ESI+): 703.3 m/z (M+H).

### Step 1: tert-butyl ((S)-2-(((3S,6S,9S)-3-((5-chloro-2-methylpyridin-3-yl)methyl)-6-isobutyl-4-methyl-2,5,8-trioxo-1,4,7-triazacyclohexadecan-9-yl)(methyl)amino)-1-cyclopropyl-2-oxoethyl)carbamate (234)

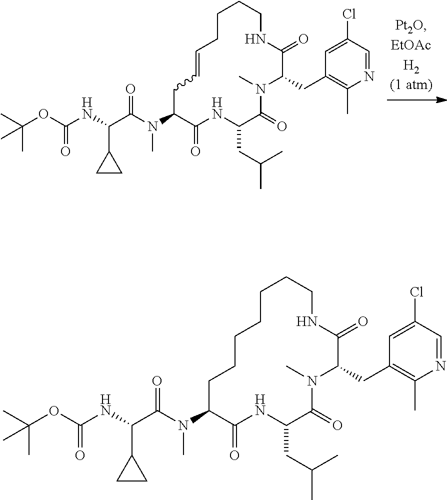 [SMILES: Cc1ncc(Cl)cc1C[C@H]1C(=O)NCCCCC=CC[C@H](N(C)C(=O)[C@@H](NC(=O)OC(C)(C)C)C2CC2)C(=O)N[C@@H](CC(C)C)C(=O)N1C ; Cc1ncc(Cl)cc1C[C@H]1C(=O)NCCCCCCC[C@H](N(C)C(=O)[C@@H](NC(=O)OC(C)(C)C)C2CC2)C(=O)N[C@@H](CC(C)C)C(=O)N1C]

233 (230.6 mg, 1 Eq, 327.9 μmol) was dissolved in EtOAc (5.0 mL) by stirring at ambient temperature. Platinum(IV) oxide (11.17 mg, 0.15 equiv., 49.18 μmol) was added to the stirring reaction mixture. The reaction was then placed under an atmosphere of hydrogen and stirred vigorously until LCMS analysis showed complete consumption of starting material. The reaction mixture was filtered over a pad of Celite. The filter was washed with EtOAc (2×50 mL). The organic solvent was concentrated to afford 234 (220 mg, 312 μmol, 95%) as an off white solid. The reaction was used without further purification. LCMS: m/z 705.3 [M+H].

### Step 2: (S)-2-amino-N-((3S,6S,9S)-3-((5-chloro-2-methylpyridin-3-yl)methyl)-6-isobutyl-4-methyl-2,5,8-trioxo-1,4,7-triazacyclohexadecan-9-yl)-2-cyclopropyl-N-methylacetamide (235)

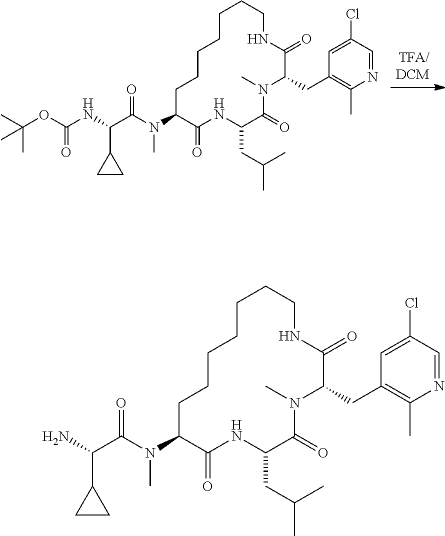 [SMILES: Cc1ncc(Cl)cc1C[C@H]1C(=O)NCCCCCCC[C@H](N(C)C(=O)[C@@H](NC(=O)OC(C)(C)C)C2CC2)C(=O)N[C@@H](CC(C)C)C(=O)N1C ; Cc1ncc(Cl)cc1C[C@H]1C(=O)NCCCCCCC[C@H](N(C)C(=O)[C@@H](N)C2CC2)C(=O)N[C@@H](CC(C)C)C(=O)N1C]

234 (231.3 mg, 1 Eq, 327.9 μmol) was dissolved in 6 mL DCM/TFA (3:1) and stirred until the deprotection was complete (monitored via LCMS). The volatile solvent was completely removed to afford a quantitative yield of 235 as the TFA salt. LCMS (ESI+): m/z 605.7 (M+H).

### Step 3: (2S,4R)—N—((S)-2-(((3S,6S,9S)-3-((5-chloro-2-methylpyridin-3-yl)methyl)-6-isobutyl-4-methyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 331)

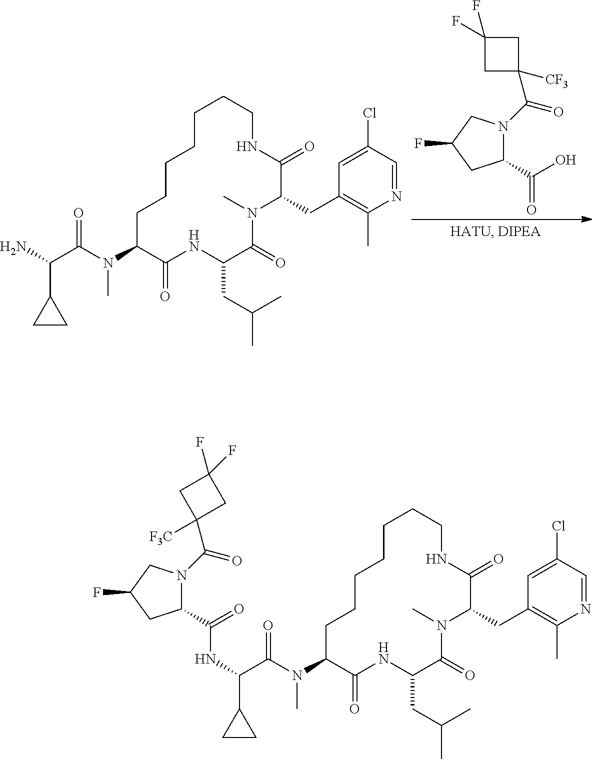

235 as the TFA salt (198.5 mg, 1 Equiv., 328.0 μmol) and Int. 141 (125.6 mg, 1.2 Equiv., 393.6 μmol) were dissolved in DMF (2.0 mL). DIPEA (254.3 mg, 343 μL, 6 Equiv., 1.968 mmol) and HATU (149.7 mg, 1.2 Equiv., 393.6 μmol) were added sequentially and the reaction was continued until the consumption of starting material (monitored via LCMS). The reaction mixture was neutralized to pH ˜7 with 1N HCl and directly purified by RP-HPLC (C18, 40% to 80% MeCN/H₂O with 0.5% formic acid). Example 331 (83.25 mg, 91.85 μmol, 28%)) was obtained as a white solid after lyophilization. LCMS (ESI+): m/z 906.6 (M+H).

g. Method 7

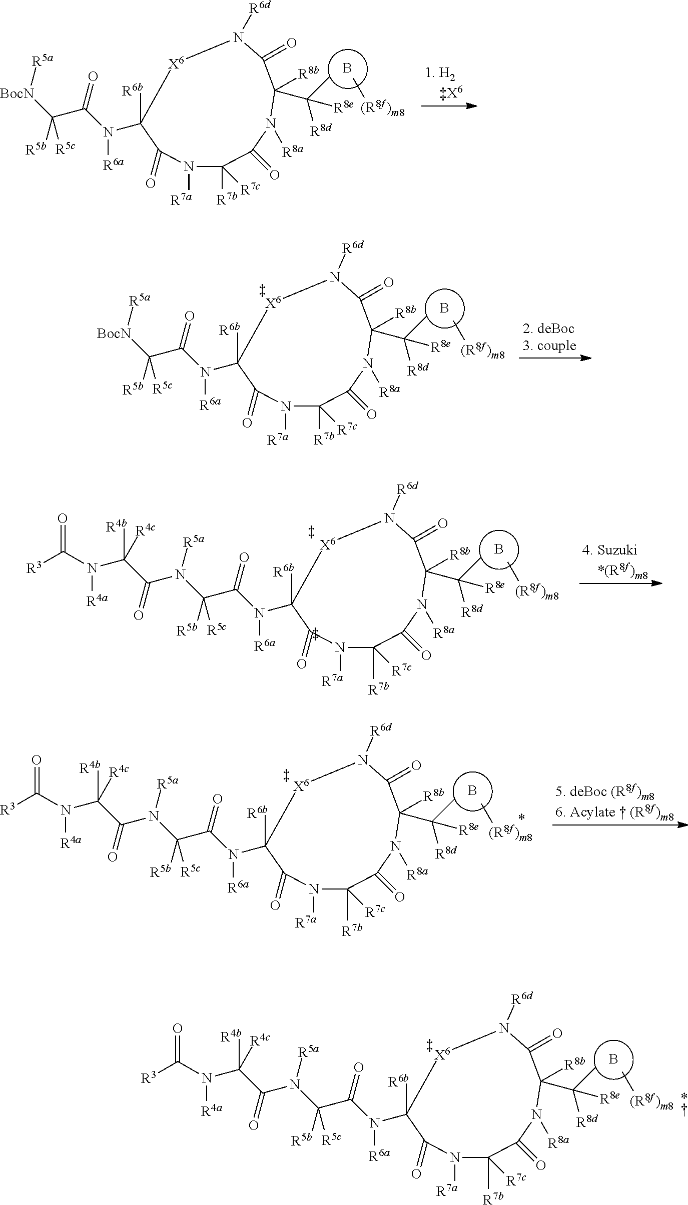

### Representative Procedure for Method 7: Example 165 and Example 58

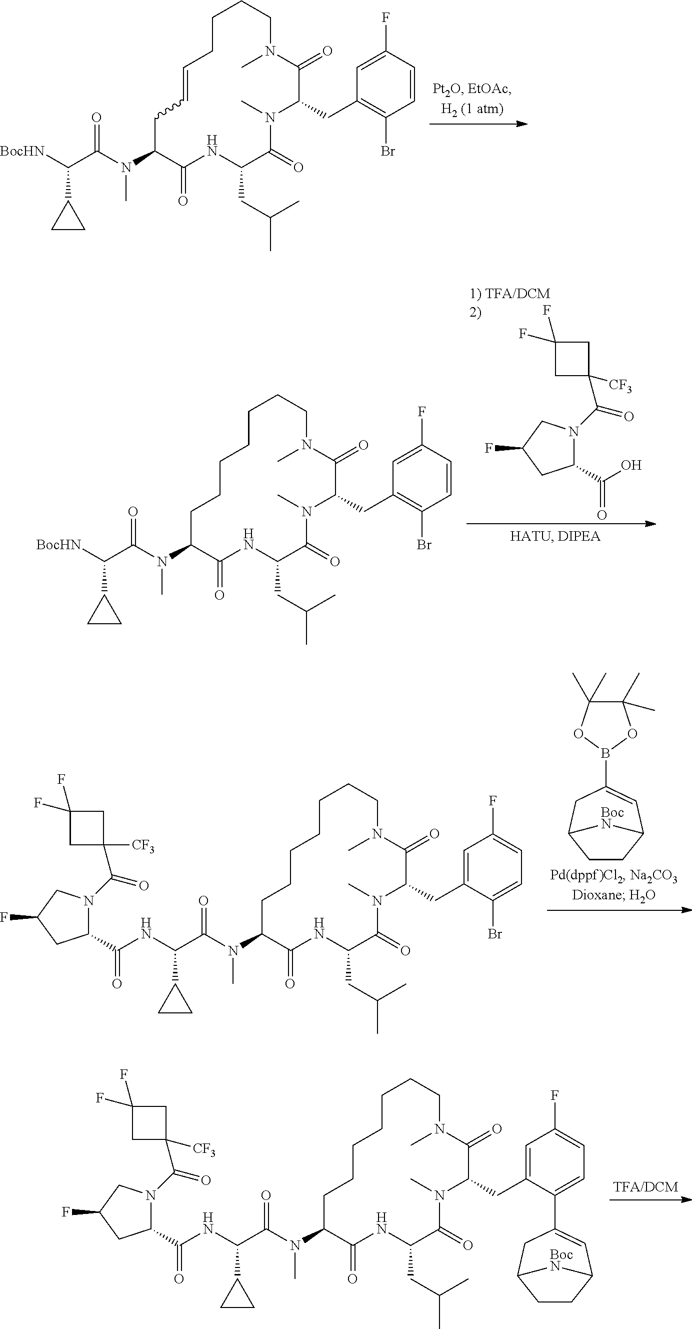

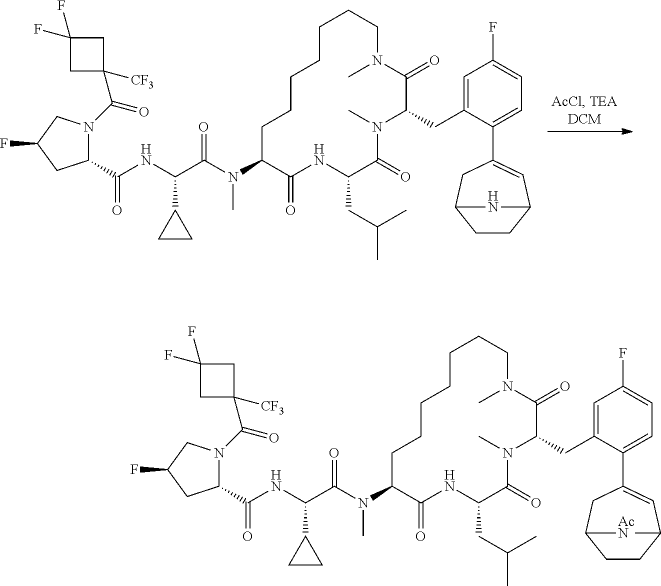

### Representative Experimental for Method 7: Example 165 and Example 58

### Step 1: tert-butyl((1S)-2-(((3S,6S,9S)-4-acetyl-3-(2-bromo-5-fluorobenzyl)-6-isobutyl-1,4-dimethyl-2,5,8-trioxo-1,414,7-triazacyclohexadecan-9-yl)(methyl)amino)-1-cyclopropyl-2-oxoethyl)carbamate (236)

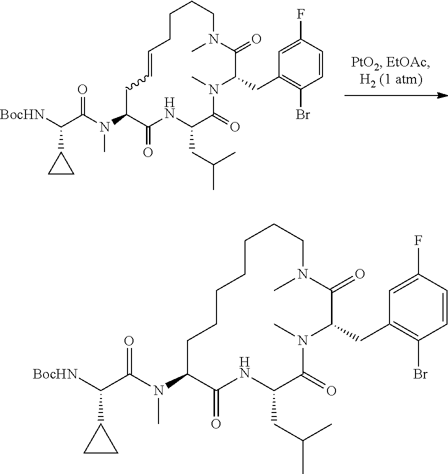 [SMILES: CC(C)C[C@@H]1NC(=O)[C@@H](N(C)C(=O)[C@@H](NC(=O)OC(C)(C)C)C2CC2)CC=CCCCCN(C)C(=O)[C@H](Cc2cc(F)ccc2Br)N(C)C1=O ; CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(F)ccc2Br)N(C)C1=O]

208 (14 g, 1 Equiv., 18 mmol) was dissolved in EtOAc (180 mL) and platinum (IV) oxide (0.33 g, 0.08 Equiv., 1.5 mmol) was added. The reaction was placed under a hydrogen atmosphere and vigorously stirred. The reaction was run rt overnight. The reaction was filtered over a bed of celite and the solvent was reduced and 236 was isolated as an off-white solid (Quant. 14.0 g). The reaction was used in the next step without any further purification. LCMS (ESI+): m/z 766.4 (M+H).

### Step 2-3: (2S,4R)—N—((S)-2-(((3S,6S,9S)-3-(2-bromo-5-fluorobenzyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (237)

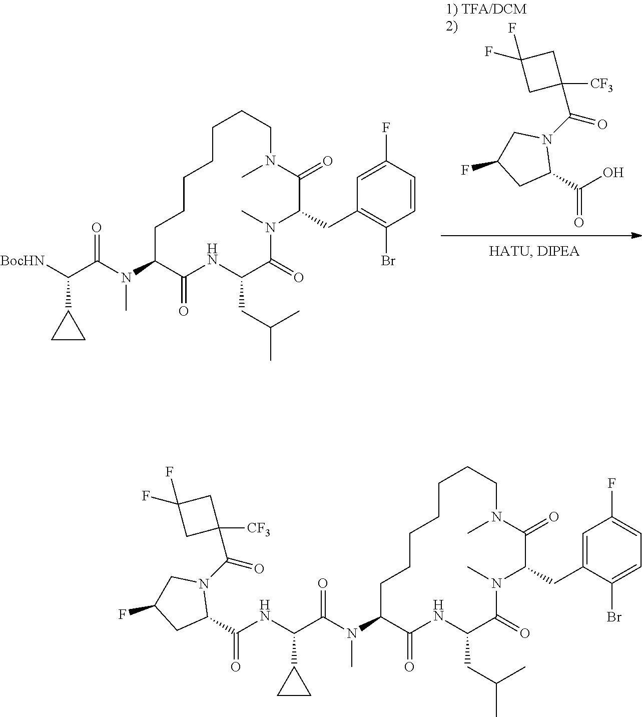 [SMILES: CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(F)ccc2Br)N(C)C1=O ; O=C(O)[C@@H]1C[C@@H](F)CN1C(=O)C1(C(F)(F)F)CC(F)(F)C1 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(F)ccc2Br)N(C)C1=O]

236 (300 mg, 1 Equiv., 391 μmol) was dissolved in 6 mL DCM/TFA (3:1) and stirred until the deprotection was complete (monitored via LCMS). The volatile solvent was completely removed to afford a quantitative yield of free amine. The crude deprotected amine was used in the next step without any further purification.

The deprotected amine, Int. 141 (137 mg, 1.1 Equiv., 430 μmol) and HATU (164 mg, 1.1 Equiv., 430 μmol) were dissolved in DMF (4 mL) and DIPEA (253 mg, 341 μL, 5 Equiv., 1.96 mmol) was added slowly. The reaction was stopped by the addition of 15 mL water upon the consumption of starting material (monitored by LCMS). The aqueous layer was extracted with EtOAc (2×10 mL). The combined organic layer was washed with brine, dried with MgSO₄, filtered, and concentrated. 237 (300 mg, 310 μmol, 79%) was isolated as an off-white solid and used without further purification. LCMS (ESI+): m/z 967.3 (M+H).

### Step 4: Tert-butyl 3-(2-(((3S,6S,9S)-9-((S)-2-cyclopropyl-2-((2S,4R)-1-(3,3-difluoro-1-(trifluoromethyl)cyclobutane-1-carbonyl)-4-fluoropyrrolidine-2-carboxamido)-N-methylacetamido)-6-isobutyl-1,4-dimethyl-2,5,8-trioxo-1,4,7-triazacyclohexadecan-3-yl)methyl)-4-fluorophenyl)-8-azabicyclo[3.2.1]oct-2-ene-8-carboxylate (238)

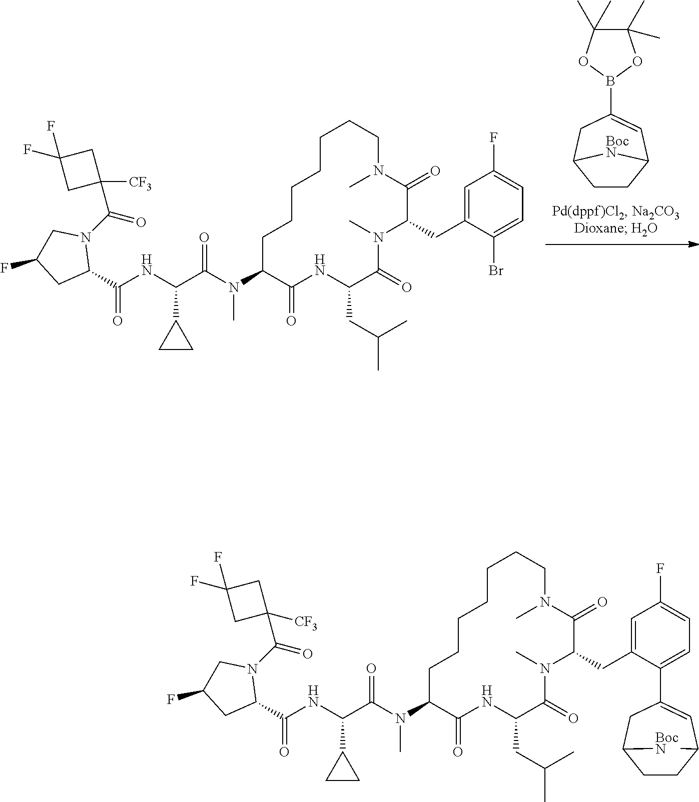 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(F)ccc2Br)N(C)C1=O ; CC(C)(C)OC(=O)N1C2C=C(B3OC(C)(C)C(C)(C)O3)CC1CC2 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(F)ccc2C2=CC3CCC(C2)N3C(=O)OC(C)(C)C)N(C)C1=O]

237 (54.0 mg, 1.2 Equiv., 161 μmol), Na₂CO₃ (47.0 mg, 222 p L, 2.0 molar, 3.3 Equiv., 443 μmol), Int. 134 (31.2 mg, 2 Equiv., 93.0 μmol) and PdCl₂(dppf)-DCM adduct (16.5 mg, 0.15 Equiv., 20.1 μmol) were dissolved in 4:1 Dioxane/Water (4 mL). The reaction was degassed for 10 min with Ar and then heated to 80° C. until the complete consumption of starting material (monitored by LCMS). The reaction mixture was diluted with water (5 mL) and extracted with EtOAc (2×10 mL). The combined organic layer was washed with brine, dried over MgSO₄, filtered, and concentrated. 208 (as a mixture of diastereomers) was used in the next reaction without any additional purification. LCMS (ESI+): m/z 1097.5 (M+H).

### Step 5: (2S,4R)—N-((1S)-2-(((3S,6S,9S)-3-(2-(8-azabicyclo[3.2.1]oct-2-en-3-yl)-5-fluorobenzyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Exampl 165)

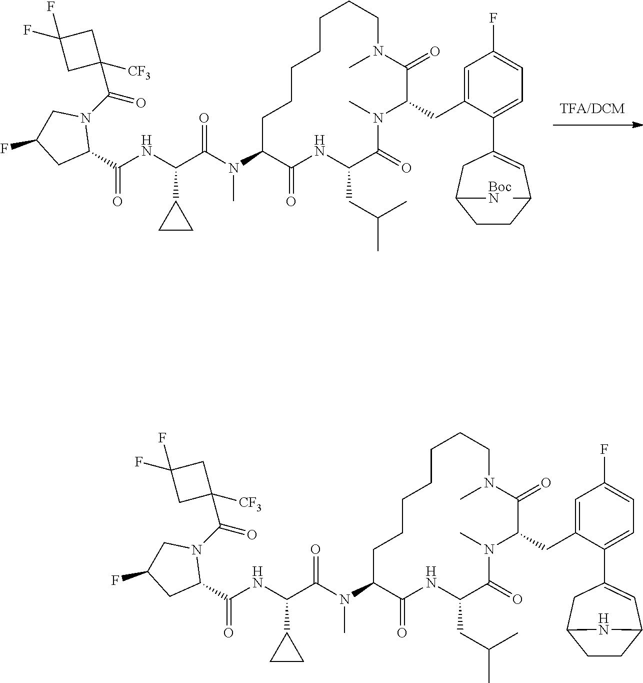 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(F)ccc2C2=CC3CCC(C2)N3C(=O)OC(C)(C)C)N(C)C1=O ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(F)ccc2C2=CC3CCC(C2)N3)N(C)C1=O]

The crude Suzuki product was dissolved in 2 mL DCM/TFA (3:1) and stirred until the deprotection was complete (monitored via LCMS). The volatile solvent was completely removed to afford the crude deprotection product. The remaining TFA was removed by co-evaporation with toluene. The material can be used without any further purification in the next reaction. A portion of the crude reaction mixture was isolated via RP-HPLC (C18, 15% to 50% MeCN/H₂O with 0.5% formic acid) to afford Example 165 (as a mixture of diastereomers, Formic Acid Salt) as a white powder after lyophilization. The remaining unpurified material was used in the next reaction directly. LCMS (ESI+): m/z 996.47 (M+H).

### Step 6: (2S,4R)—N-((1S)-2-(((3S,6S,9S)-3-(2-(8-azabicyclo[3.2.1]oct-2-en-3-yl)-5-fluorobenzyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 58)

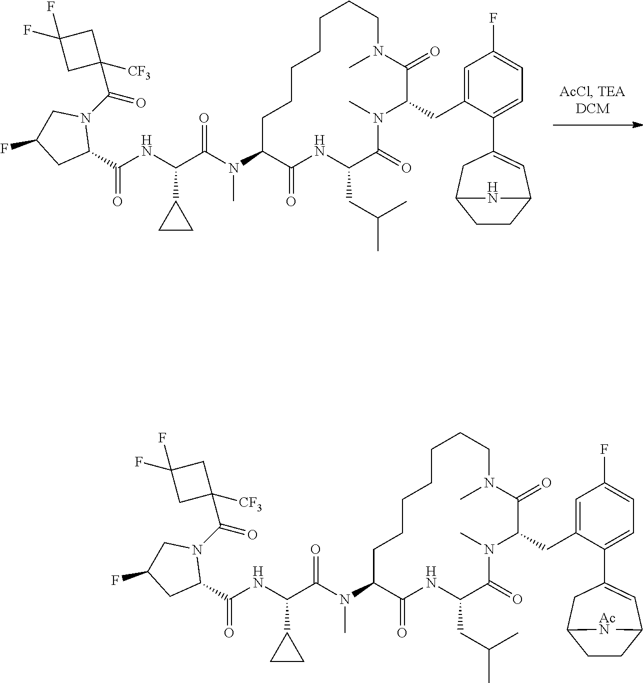

Example 165 was dissolved in DCM (4 mL), TEA was added to adjust the pH to ˜8.0 (˜4 equiv.). Next, the reaction was cooled with an ice bath and AcCl (2.4 mg, 2.1 μL, 1 Equiv., 30 μmol) was added. Upon the complete consumption of starting material, the reaction was concentrated and directly purified by RP-HPLC (C18, 50% to 80% MeCN/H₂O with 0.5% formic acid) to afford Example 58 as a mixture of diastereomers (13 mg, 12 μmol, 40%) as a white solid after lyophilization. LCMS (ESI+): m/z 1038.59 (M+H).

h. Method 8

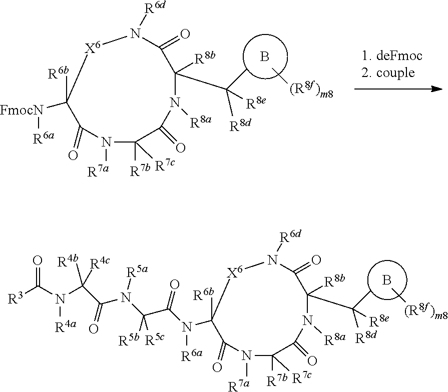

### Representative Experimental for Method 8: Example 1

### Step 1: N—((S)-1-(((S)-1-(((3S,6S,9S)-3-(2,5-dichlorobenzyl)-6-isobutyl-4-methyl-2,5,8-trioxo-1,4,7-triazacyclohexadec-11-en-9-yl)amino)-1-oxopropan-2-yl)amino)-1-oxobutan-2-yl)-1-(trifluoromethyl)cyclopropane-1-carboxamide (Example 1)

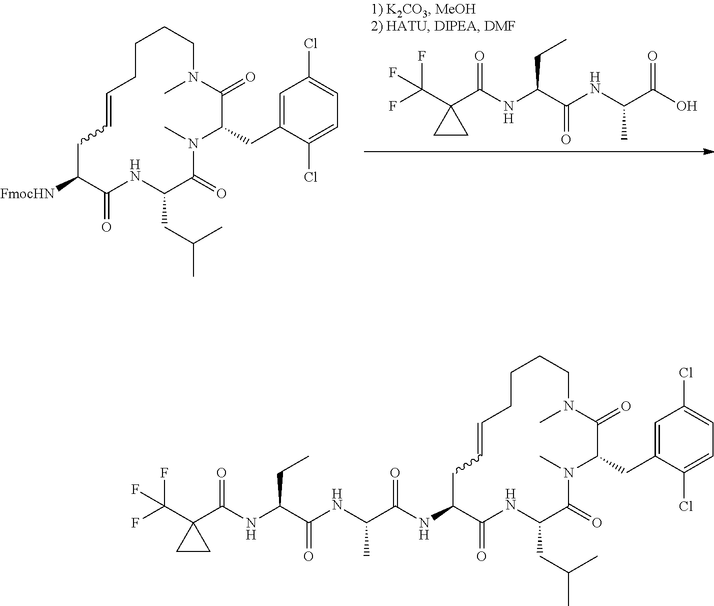

218 (30 mg, 1 Equiv., 41 μmol) was dissolved in MeOH (2 ml), and K₂CO₃ (7.0 mg, 1.2 Equiv., 51 μmol) was added while stirring. The reaction continued until the complete removal of the FMOC protecting group (monitored by LCMS). Upon completion, the reaction was concentrated and DCE (2×4 mL) was used to dissolve the deprotected amine. The DCE was filtered and concentrated. The crude amine was used directly in the next reaction.

The deprotected amine from above, Int. 34 (15 mg, 1.2 Equiv., 48 μmol), and HATU (16 mg, 1 Equiv., 41 μmol) were dissolved in DMF (2 mL). DIPEA (35.5 μL, 1 equiv., 205 μmol) was added and the reaction was continued until the complete consumption of starting material (monitored by LCMS). Upon completion, the reaction mixture was neutralized with minimal 1N HCl and directly purified by RP-HPLC (C18, 40% to 70% MeCN/H₂O with 0.5% formic acid). Example 1 (4.5 mg, 5.6 μmol, 14%) was isolated after lyophilization as a white solid. LCMS (ESI+): m/z 803.4 (M+H).

i. Method 9

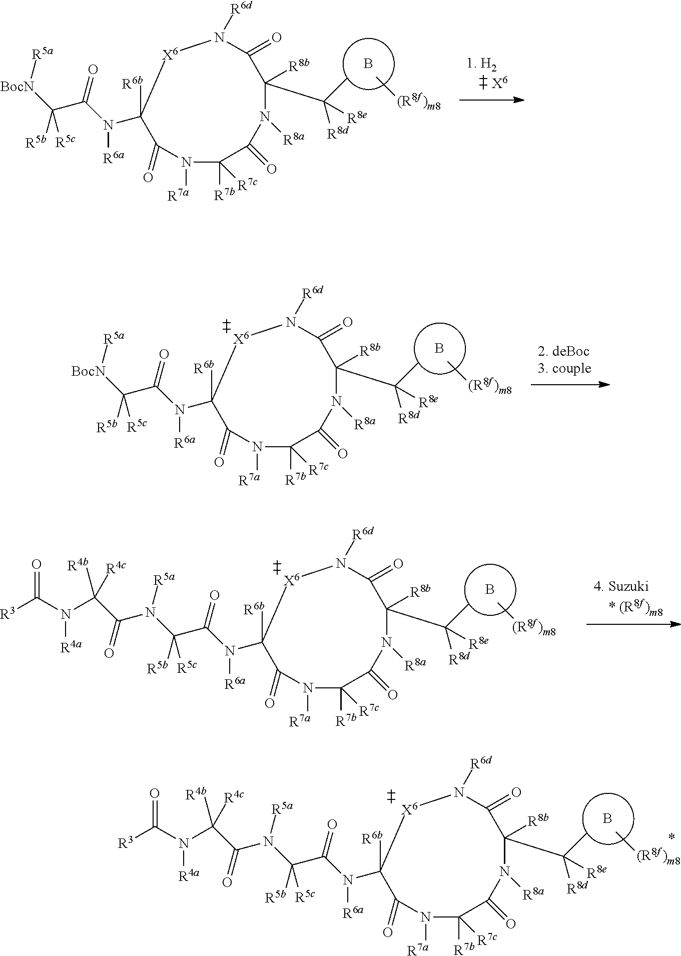

### Representative Procedure for Method 9: Example 359

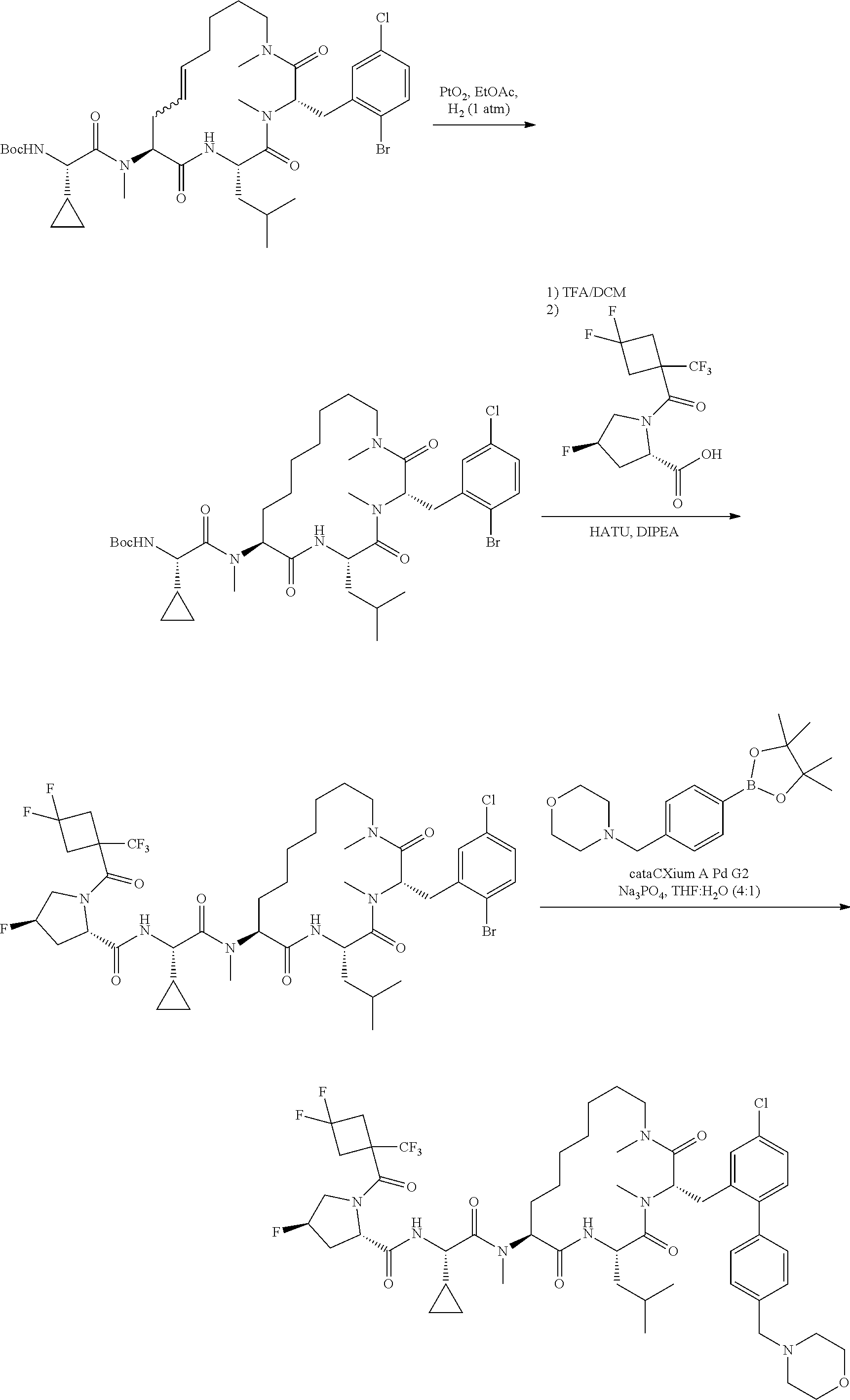

### Representative Expiremental for Method 9: Example 359

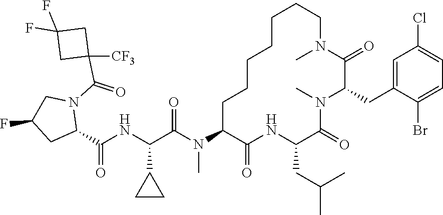 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2Br)N(C)C1=O]

The starting material for Method 9, 239, was synthesized using identical procedures for steps 1-3 of Method 7 using 226 and Int. 141 LCMS (ESI): m/z 985.5 [M+H].

### Step 4: (2S,4R)—N—((S)-2-(((3S,6S,9S)-3-((4-chloro-4′-(morpholinomethyl)-[1,1′-biphenyl]-2-yl)methyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 359)

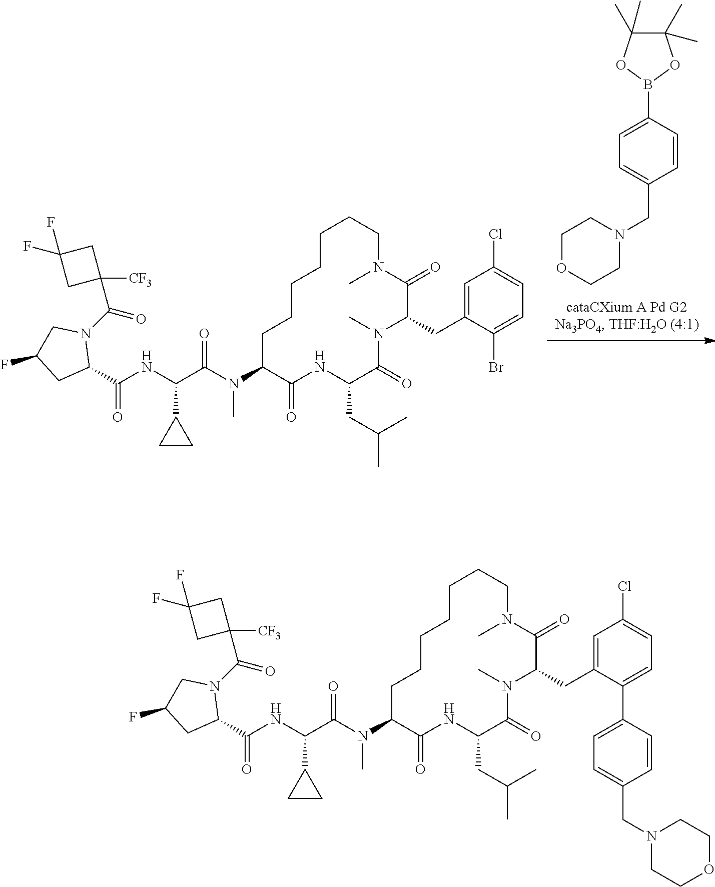

239 (125 mg, 1 Equiv. 127 mol), 4-(4-(4,4,5,5-tetramethyl-1,3,2-dioxaborolan-2-yl)benzyl)morpholine (38.5 mg, 1 Equiv. 127 μmol), and Chloro[(di(1-adamantyl)-N-butylphosphine)-2-(2-aminobiphenyl)]palladium(II) (8.49 mg, 0.1 Equiv. 12.7 μmol) were dissolved in 1.0 mL THF and Na₃PO₄ (267 μL, 1.0 molar, 2.1 Equiv. 267 μmol). The mixture was degassed with a stream of Argon, then sealed and stirred at 70° C. until the complete consumption of starting material (monitored by LCMS). The reaction was then cooled to room temperature and adjusted to pH ˜7 with 1N HCl. The reaction mixture was filtered and directly purified by RP-HPLC (C18, 30% to 60% MeCN/H₂O with 0.5% formic acid). Example 359 (48.8 mg, 45.2 μmol, 35.6%) was isolated as a white solid after lyophilization. LCMS (ESI): m/z 1080.3 [M+H].

j. Method 10

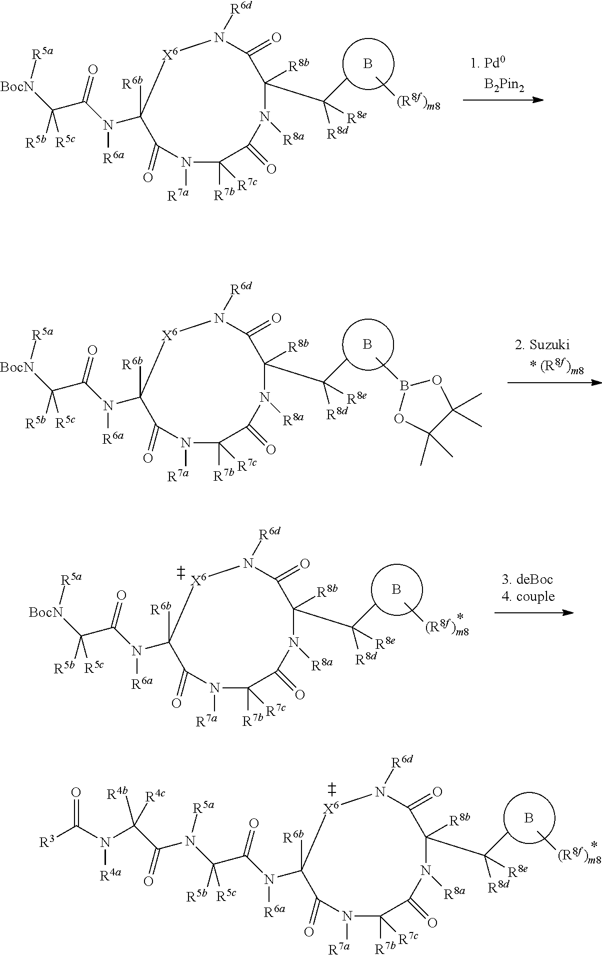

### Representative Procedure for Method 10: Example 382

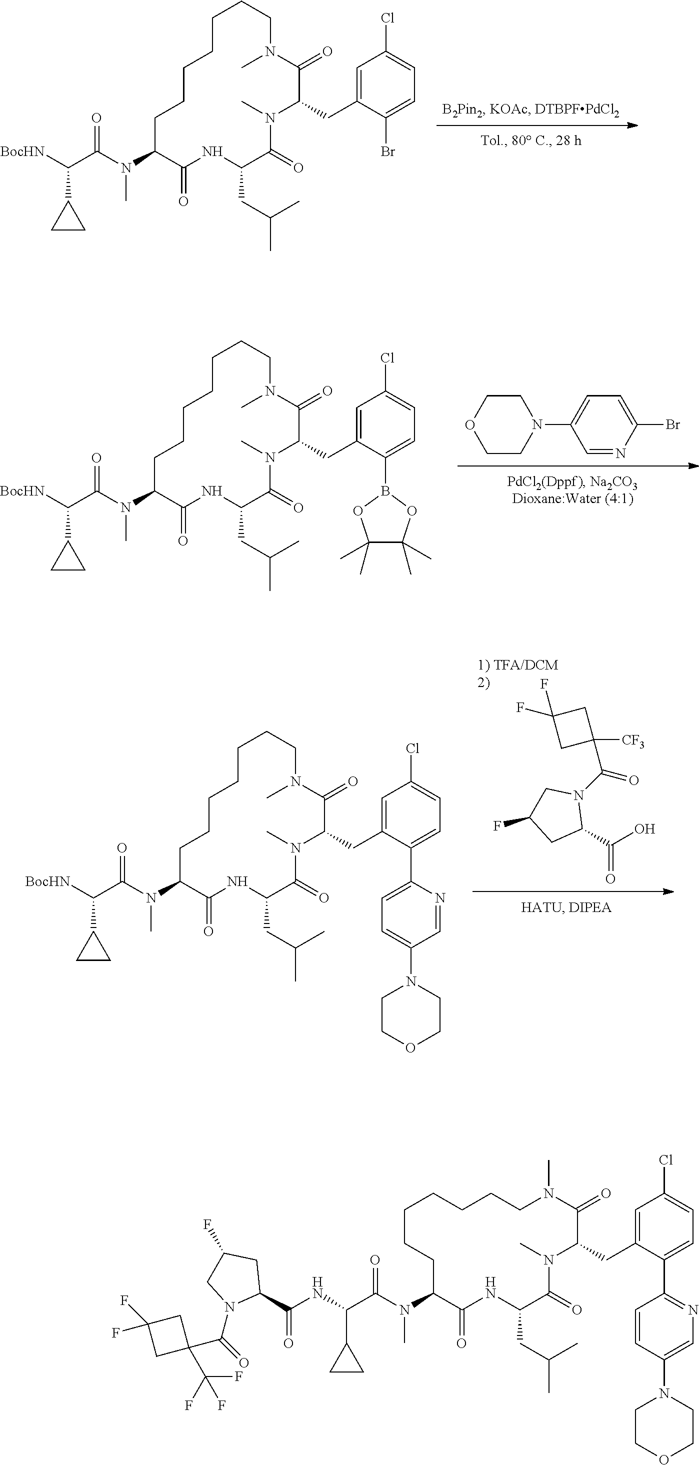

### Step 1: tert-butyl ((S)-2-(((3S,6S,9S)-3-(5-chloro-2-(4,4,5,5-tetramethyl-1,3,2-dioxaborolan-2-yl)benzyl)-6-isobutyl-1,4-dimethyl-2,5,8-trioxo-1,4,7-triazacyclohexadecan-9-yl)(methyl)amino)-1-cyclopropyl-2-oxoethyl)carbamate (240)

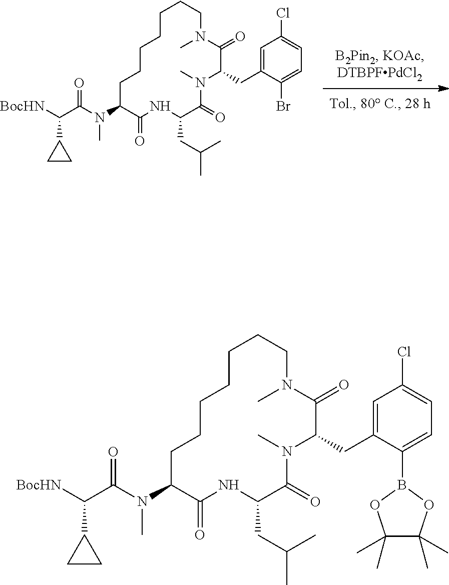 [SMILES: CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2Br)N(C)C1=O ; CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2B2OC(C)(C)C(C)(C)O2)N(C)C1=O]

To a solution of macrocycle 227 (300 mg, 383.03 μmol, 1 eq) and B₂Pin₂ (194.53 mg, 766.06 μmol, 2 eq) in Toluene (5 mL) was added KOAc (75.18 mg, 766.06 μmol, 2 eq) and ditert-butyl(cyclopentyl)phosphane; dichloropalladium; iron (24.96 mg, 38.30 μmol, 0.1 eq) under N₂ atmosphere. The reaction mixture was heated to 80° C. and stirred for 28 hr. The reaction mixture was cooled to room temperature and quenched by water (10 mL), extracted with EtOAc (10 mL×2). The combined organic layer was washed with brine (15 mL), dried over Na₂SO₄, filtered, and concentrated under reduced pressure to give a residue. The residue was first purified by flash column chromatography (SiO₂, 0 to 50% EtOAc/Pet. Ether). Then by prep-HPLC (C18, using H₂O (10 mM NH₄HCO₃) with MeCN 50 to 80% over 8.0 min). The product was lyophilized to give 240 (20 mg, 5.78 μmol, 1.51% yield) as a white solid. LCMS (ESI): 730.6 (M−Boc)

### Step 2: tert-butyl ((S)-2-(((3S,6S,9S)-3-(5-chloro-2-(5-morpholinopyridin-2-yl)benzyl)-6-isobutyl-1,4-dimethyl-2,5,8-trioxo-1,4,7-triazacyclohexadecan-9-yl)(methyl)amino)-1-cyclopropyl-2-oxoethyl)carbamate (241)

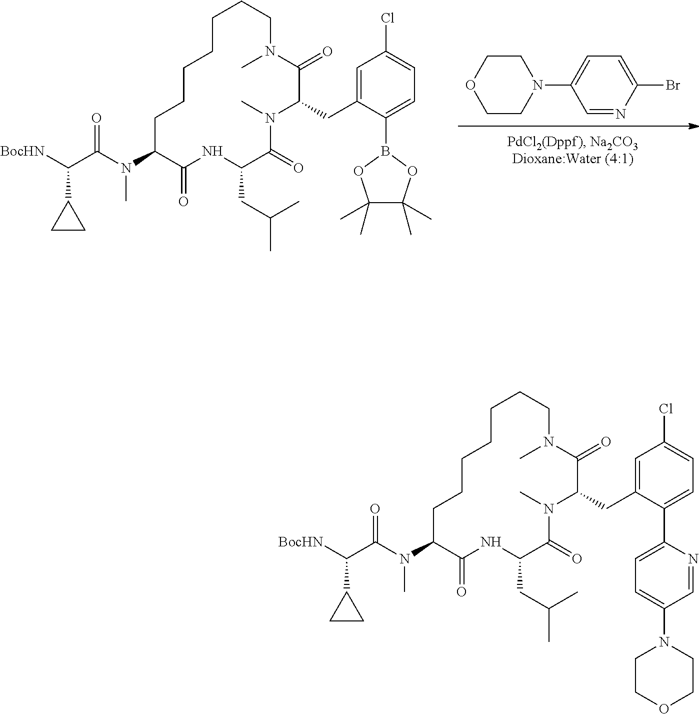 [SMILES: CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2B2OC(C)(C)C(C)(C)O2)N(C)C1=O ; Brc1ccc(N2CCOCC2)cn1 ; CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2-c2ccc(N3CCOCC3)cn2)N(C)C1=O]

240 200 mg, 1 Equiv. 241 μmol), 4-(6-bromopyridin-3-yl)morpholine (82.0 mg, 1.4 Equiv. 337 μmol), and PdCl2(dppf) (19.7 mg, 0.1 Equiv. 24.1 μmol) were dissolved in mixture of 1,4-Dioxane (1.3 mL) and 2M Na₂CO₃ (58.7 mg, 277 μL, 2.3 Equiv. 554 μmol). The reaction was degassed with a stream of Ar then heated to 100° C. for 6 hr. The reaction was then cooled and adjusted to pH˜7 with Formic Acid. The crude reaction mixture was then diluted in MeOH (2 mL), filtered, and directly purified by RP-HPLC (C18, 50% to 100% MeCN/H₂O with 0.5% formic acid) to obtain 241 (95 mg, 0.11 mmol, 46%) as an off-white powder after lyophilization. LCMS (ESI): m/z 966.3 [M+H].

### Steps 3-4: Example 382

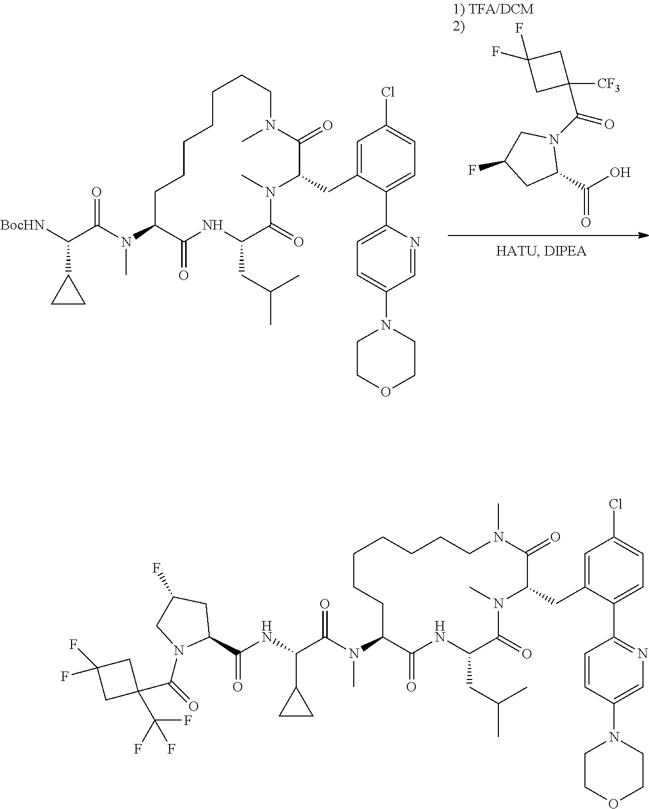 [SMILES: CC(C)C[C@@H]1NC(=O)[C@@H](N(C)C(=O)[C@@H](NC(=O)OC(C)(C)C)C2CC2)CCCCCCCN(C)C(=O)[C@H](Cc2cc(Cl)ccc2-c2ccc(N3CCOCC3)cn2)N(C)C1=O ; O=C(O)[C@@H]1C[C@@H](F)CN1C(=O)C1(C(F)(F)F)CC(F)(F)C1 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCOCC3)cn2)N(C)C1=O]

Example 382 was synthesized using identical procedures for Method 4 (Steps 3-4) using 241 and Int. 141. LCMS (ESI): m/z 1068.2 [M+H].

k. Method 11

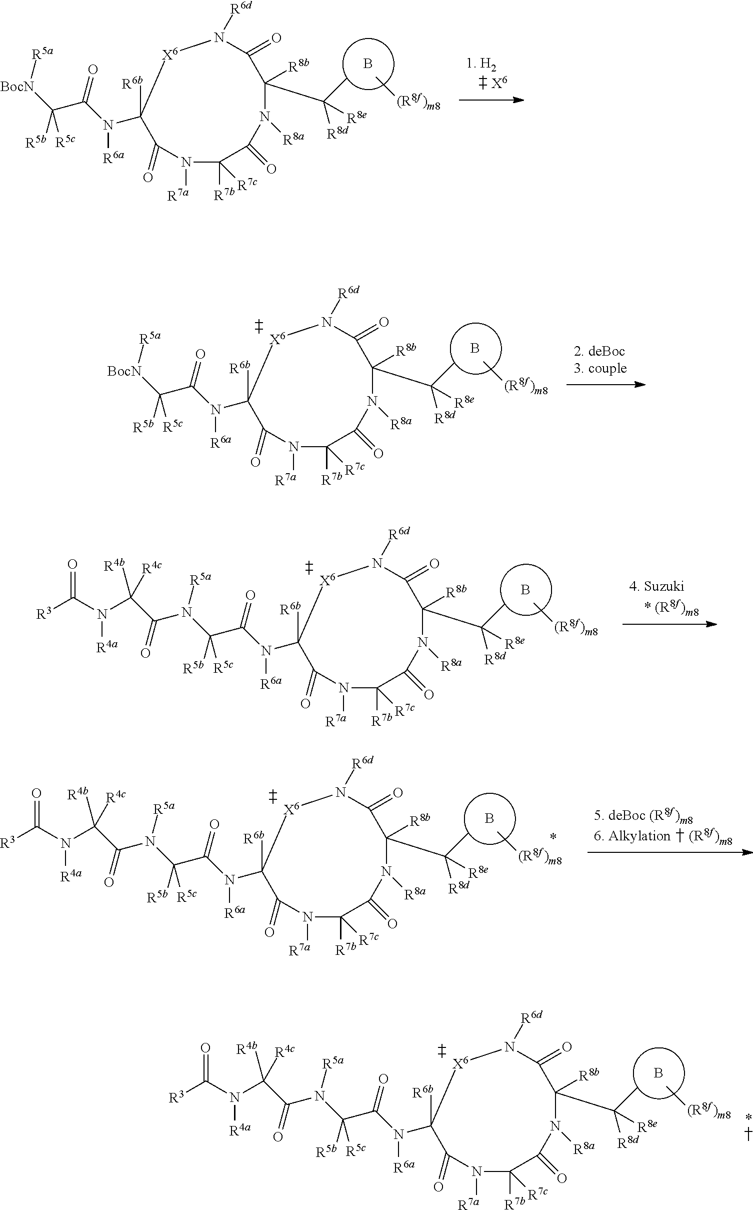

### Representative Procedure for Method 11: Example 383

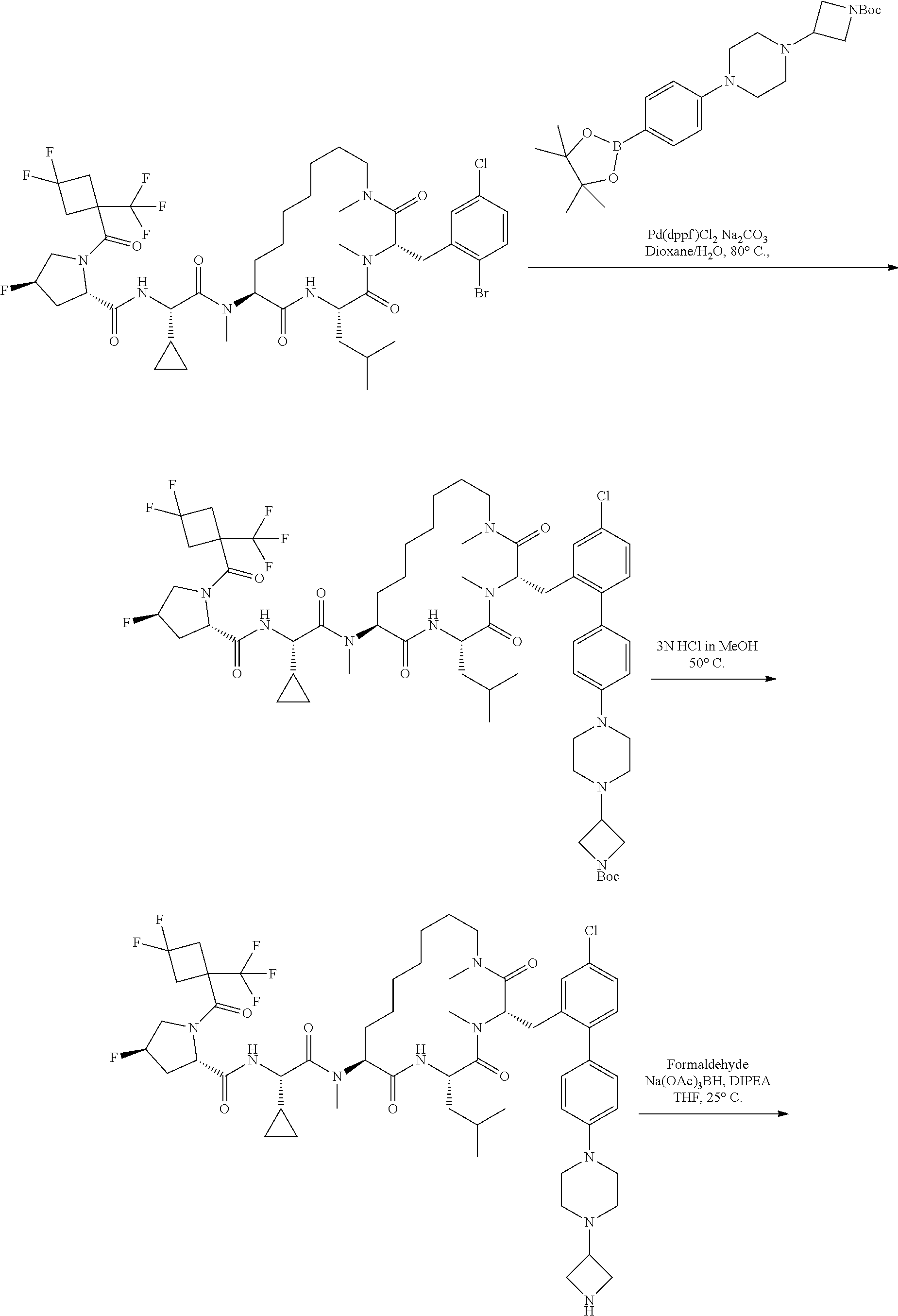

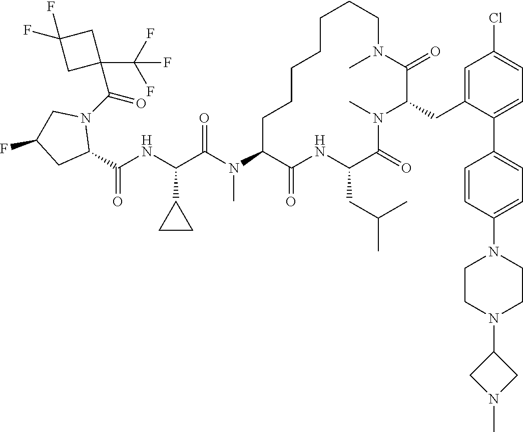 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CN(C)C4)CC3)cc2)N(C)C1=O]

### Step 1: tert-butyl 3-(4-(4′-chloro-2′-(((3S,6S,9S)-9-((S)-2-cyclopropyl-2-((2S,4R)-1-(3,3-difluoro-1-(trifluoromethyl)cyclobutane-1-carbonyl)-4-fluoropyrrolidine-2-carboxamido)-N-methylacetamido)-6-isobutyl-1,4-dimethyl-2,5,8-trioxo-1,4,7-triazacyclohexadecan-3-yl)methyl)-[1,1′-biphenyl]-4-yl)piperazin-1-yl)azetidine-1-carboxylate (242)

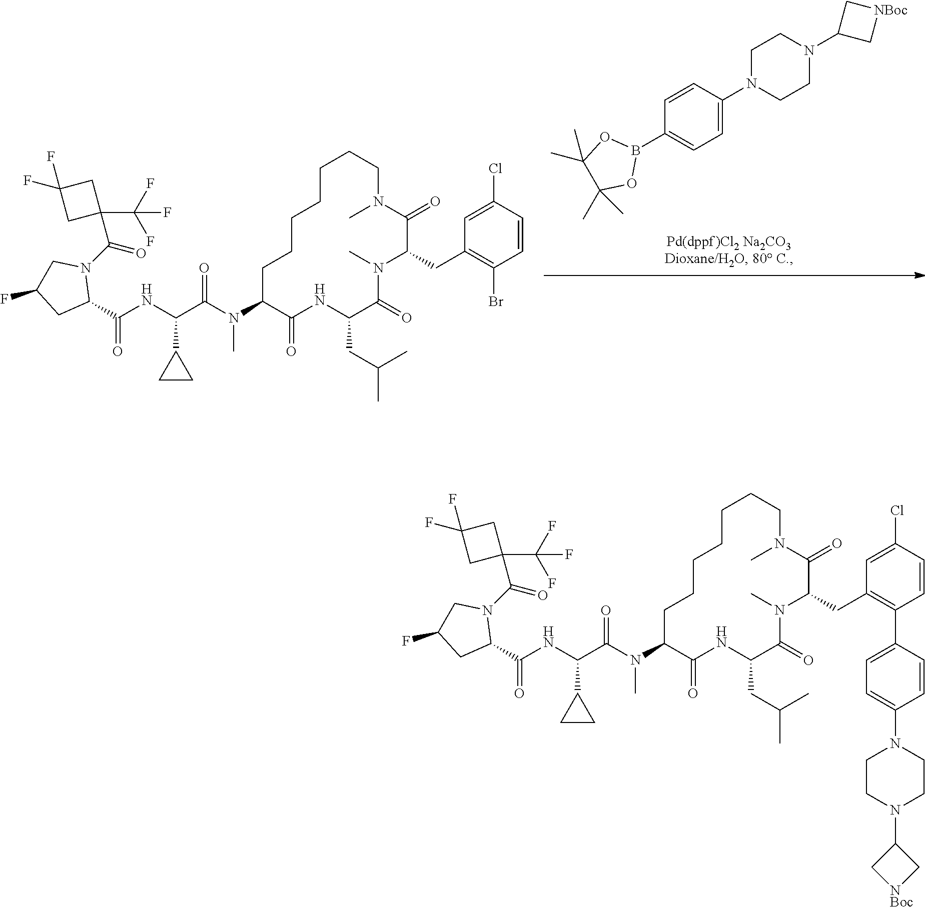 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2Br)N(C)C1=O ; CC(C)(C)OC(=O)N1CC(N2CCN(c3ccc(B4OC(C)(C)C(C)(C)O4)cc3)CC2)C1 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CN(C(=O)OC(C)(C)C)C4)CC3)cc2)N(C)C1=O]

239 (550 mg, 1 Equiv. 559 μmol), Int. 193 (496 mg, 2 Equiv. 1.12 mmol), Na₂CO₃ (237 mg, 4 Equiv. 2.24 mmol) and PdCl₂(dppf) (45.6 mg, 0.1 Equiv. 55.9 μmol) were dissolved in Dioxane:Water (4:1) (5.59 mL). The reaction was degassed for 10 min with Ar and then heated to 80° C. until the complete consumption of starting material (monitored by LCMS). The reaction mixture was diluted with water (10 mL) and extracted with EtOAc (2×10 mL). The combined organic layer was washed with brine, dried over MgSO₄, filtered, and concentrated. The crude reaction mixture was isolated via RP-HPLC (C18, 30% to 80% MeCN/H₂O with 0.5% formic acid) to afford 242 (400 mg, 328 μmol, 58.6%) as a white powder after lyophilization. LCMS (ESI+): m/z 1220.7 (M+H).

### Step 2: (2S,4R)—N—((S)-2-(((3S,6S,9S)-3-((4′-(4-(azetidin-3-yl)piperazin-1-yl)-4-chloro-[1,1′-biphenyl]-2-yl)methyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (243)

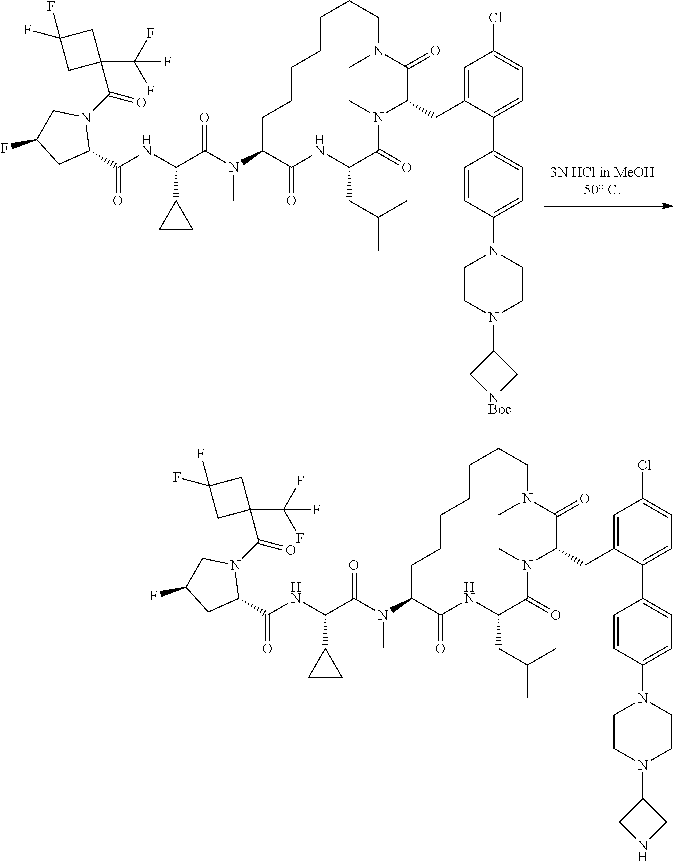 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CN(C(=O)OC(C)(C)C)C4)CC3)cc2)N(C)C1=O ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CNC4)CC3)cc2)N(C)C1=O]

242 (400 mg, 328 μmol, 1 Eq) was dissolved in 2 mL 3N HCl in MeOH and stirred at 50° C. until the deprotection was complete (monitored via LCMS). The volatile solvent was completely removed to afford the crude deprotection product, 243, as the HCl salt. The material can be used without any further purification in the next reaction. LCMS 1120.7 (ESI+): m/z (M+H).

### Step 3: (2S,4R)—N—((S)-2-(((3S,6S,9S)-3-((4-chloro-4′-(4-(1-methylazetidin-3-yl)piperazin-1-yl)-[1,1′-biphenyl]-2-yl)methyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide Example 383

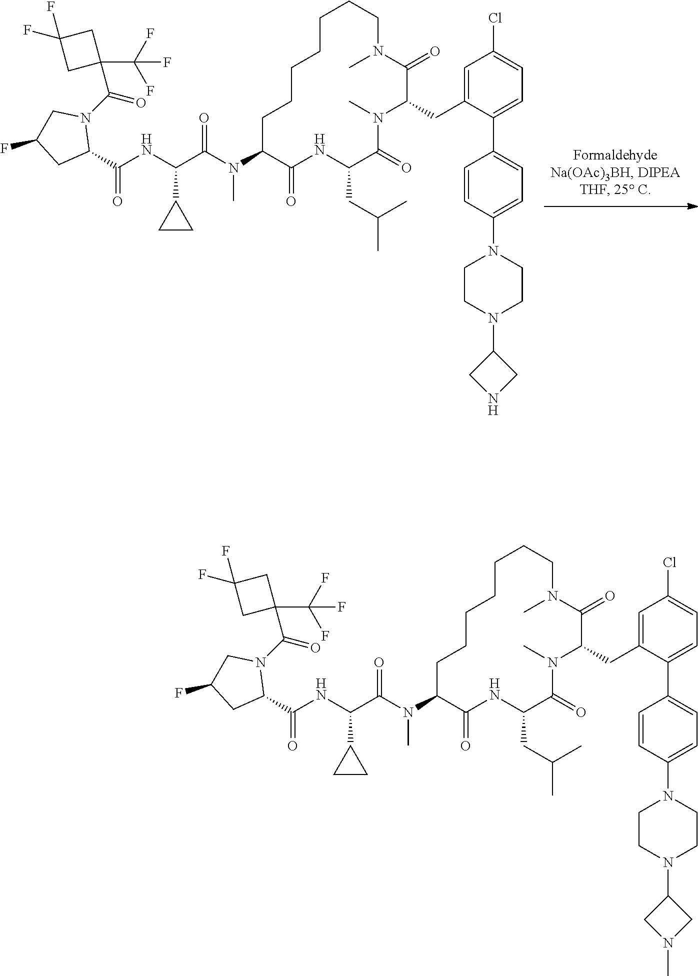 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CNC4)CC3)cc2)N(C)C1=O ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)ccc2-c2ccc(N3CCN(C4CN(C)C4)CC3)cc2)N(C)C1=O]

243 (300 mg, 1 Equiv. 268 μmol), formaldehyde (43.5 mg, 39.9 μL, 37% Wt, 2 Equiv. 535 μmol) and DIPEA (138 mg, 187 μL, 4 Equiv. 1.07 mmol) was dissolved in THF (2.68 mL) stirred at 25° C. for 15 mins. Then, sodium triacetoxyborohydride (113 mg, 2 Equiv. 535 μmol) was added portion-wise to the reaction mixture. The reaction was carried on at 25° C. for 1 hour. The starting material was completely consumed (monitored by LCMS). The reaction mixture was diluted with water (15 mL) and extracted with EtOAc (2×15 mL). The combined organic layer was washed with brine, dried over MgSO₄, filtered, and concentrated. The crude reaction mixture was purified via RP-HPLC (C18, 30% to 70% MeCN/H₂O with 0.5% formic acid) to afford Example 383 (150 mg, 132 μmol, 49.4%) as a white powder after lyophilization. LCMS (ESI+): m/z 1135.25 (M+H).

l. Method 12

### Generic Procedure for Method 12

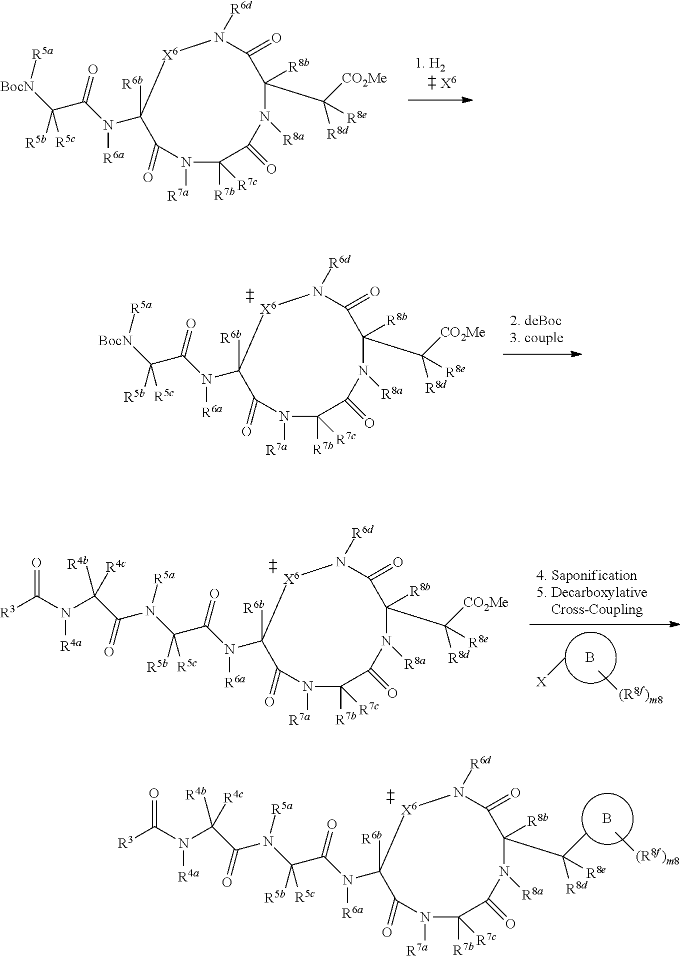

### Representative Procedure for Method 12: Example 392

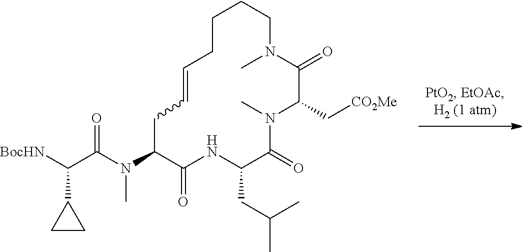 [SMILES: COC(=O)C[C@H]1C(=O)N(C)CCCCC=CC[C@H](N(C)C(=O)[C@@H](NC(=O)OC(C)(C)C)C2CC2)C(=O)N[C@@H](CC(C)C)C(=O)N1C]

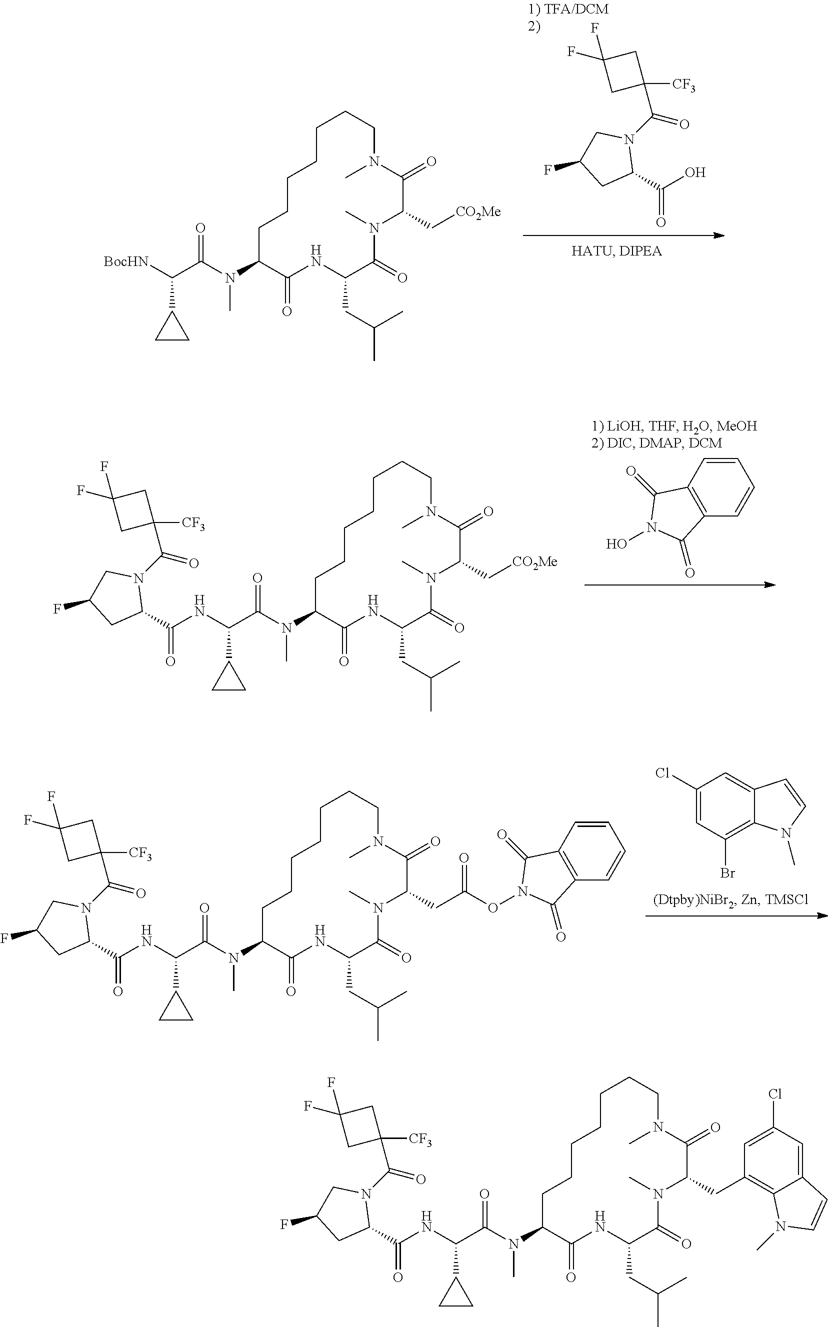

### Representative Experimental for Method 12: Example 392

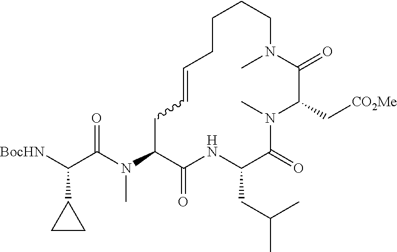 [SMILES: COC(=O)C[C@H]1C(=O)N(C)CCCCC=CC[C@H](N(C)C(=O)[C@@H](NC(=O)OC(C)(C)C)C2CC2)C(=O)N[C@@H](CC(C)C)C(=O)N1C]

The starting material for Method 12, 244, was synthesized using Method A with int. 206. LCMS (ESI): m/z 652.4 (M+H⁺).

### Steps 1-3: Methyl 2-((3S,6S,9S)-9-((S)-2-cyclopropyl-2-((2S,4R)-1-(3,3-difluoro-1-(trifluoromethyl)cyclobutane-1-carbonyl)-4-fluoropyrrolidine-2-carboxamido)-N-methylacetamido)-6-isobutyl-1,4-dimethyl-2,5,8-trioxo-1,4,7-triazacyclohexadecan-3-yl)acetate (245)

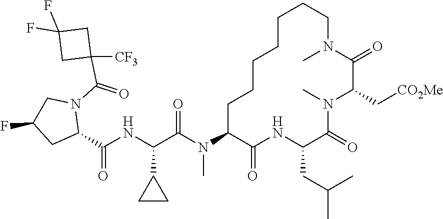 [SMILES: COC(=O)C[C@H]1C(=O)N(C)CCCCCCC[C@H](N(C)C(=O)[C@@H](NC(=O)[C@@H]2C[C@@H](F)CN2C(=O)C2(C(F)(F)F)CC(F)(F)C2)C2CC2)C(=O)N[C@@H](CC(C)C)C(=O)N1C]

245 was synthesized using identical procedures for steps 1-3 of Method 7 using 244 and Int. 141. LCMS (ESI): m/z 854.1 (M+H⁺).

### Steps 4-5: 1,3-Dioxoisoindolin-2-yl 2-((3S,6S,9S)-9-((S)-2-cyclopropyl-2-((2S,4R)-1-(3,3-difluoro-1-(trifluoromethyl)cyclobutane-1-carbonyl)-4-fluoropyrrolidine-2-carboxamido)-N-methylacetamido)-6-isobutyl-1,4-dimethyl-2,5,8-trioxo-1,4,7-triazacyclohexadecan-3-yl)acetate (246)

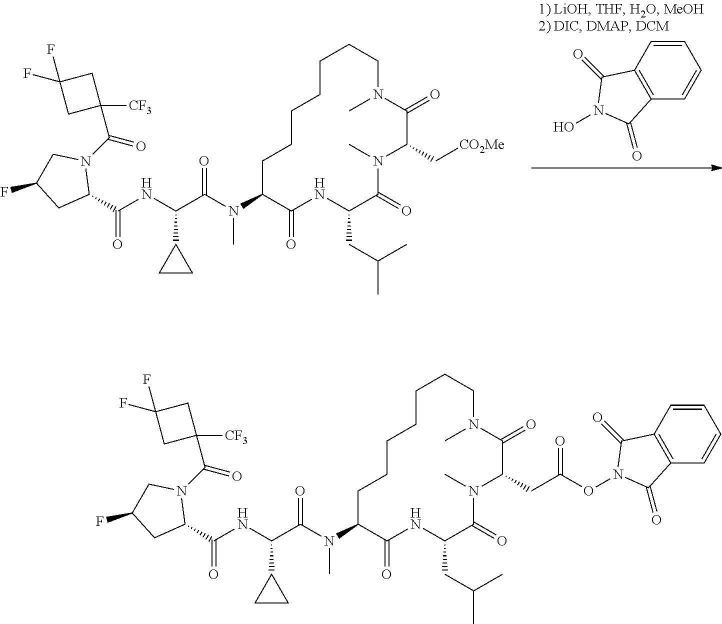 [SMILES: COC(=O)C[C@H]1C(=O)N(C)CCCCCCC[C@H](N(C)C(=O)[C@@H](NC(=O)[C@@H]2C[C@@H](F)CN2C(=O)C2(C(F)(F)F)CC(F)(F)C2)C2CC2)C(=O)N[C@@H](CC(C)C)C(=O)N1C ; O=C1c2ccccc2C(=O)N1O ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](CC(=O)ON2C(=O)c3ccccc3C2=O)N(C)C1=O]

245 (2.0 g, 1 Eq, 2.345 mmol), was dissolved in THF (16.4 mL). In a separate vial, LiOH (112.3 mg, 2 Eq, 4.690 mmol) was dissolved in Water (10.9 mL) and the LiOH solution was added to the stirring THF solution. MeOH (˜1 mL) was added until the solution became homogeneous. The reaction was stirred at 25° C. until the complete consumption of starting material (monitored by LCMS). upon completion, the solution was acidified to pH˜3 with 1N HCl. The acidified mixture was diluted with water (50 mL) and extracted with EtOAc (2×50 mL). The combined organic layer was washed with brine, dried over MgSO₄, filtered, and concentrated. The crude carboxylic acid product was used directly in the next reaction. LCMS (ESI): m/z 839.3 (M+H⁺).

The crude carboxylic acid (590 mg, 1 Eq, 703 μmol), 2-hydroxyisoindoline-1,3-dione (126 mg, 1.1 Eq, 774 μmol) and DMAP (8.59 mg, 0.1 Eq, 70.3 mo) were dissolved in DCM (4.0 mL) and DIC (97.6 mg, 121 μL, 1.1 Eq, 774 μmol) was added. the reaction was stirred until the consumption of starting material was observed (TLC 50% EtOAc in hexane). The reaction was filtered over a pad of SiO₂ which was washed with EtOAc (2×10 mL). The organic solvent was removed and the product, 246 (692 mg, 703 μmol, quant.), was used without further purification. Product decomposed to the carboxylic acid on LCMS.

### Step 6: (2S,4R)—N—((S)-2-(((3S,6S,9S)-3-((5-chloro-1-methyl-1H-indol-7-yl)methyl)-6-isobutyl-1,4-dimethyl-2,5,8-trioxo-1,4,7-triazacyclohexadecan-9-yl)(methyl)amino)-1-cyclopropyl-2-oxoethyl)-1-(3,3-difluoro-1-(trifluoromethyl)cyclobutane-1-carbonyl)-4-fluoropyrrolidine-2-carboxamide (Example 392)

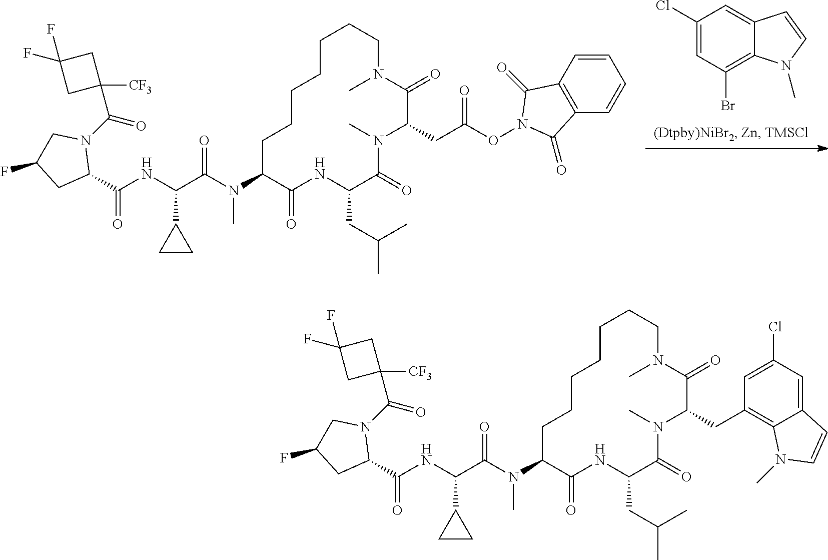 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](CC(=O)ON2C(=O)c3ccccc3C2=O)N(C)C1=O ; Cn1ccc2cc(Cl)cc(Br)c21 ; CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCN(C)C(=O)[C@H](Cc2cc(Cl)cc3ccn(C)c23)N(C)C1=O]

DMA (2.0 mL) was added to a vial containing 246 (175 mg, 1 Eq, 178 μmol), 7-bromo-5-chloro-1-methyl-1H-indole (87.0 mg, 2 Eq, 356 μmol). Then, TMS-C₁ (4.3 mg, 5.0 μL, 0.22 Eq, 39 μmol) was added and the mixture was degassed with a stream of Ar for 5 min. (Dtpby)NiBr₂ (21.6 mg, 0.25 Eq, 44.5 μmol) was added, while degassing was continued for an additional 5 min. Next, the vial was quickly capped, and the mixture was stirred overnight at ambient temperature. LCMS showed the consumption of starting material, and the reaction was filtered and directly purified by RP-HPLC. Example 392 was isolated as a fluffy white solid (15.7 mg, 16.4 μmol, 9.21%). LCMS (ESI): m/z 958.48 (M+H⁺).

m. Method 13

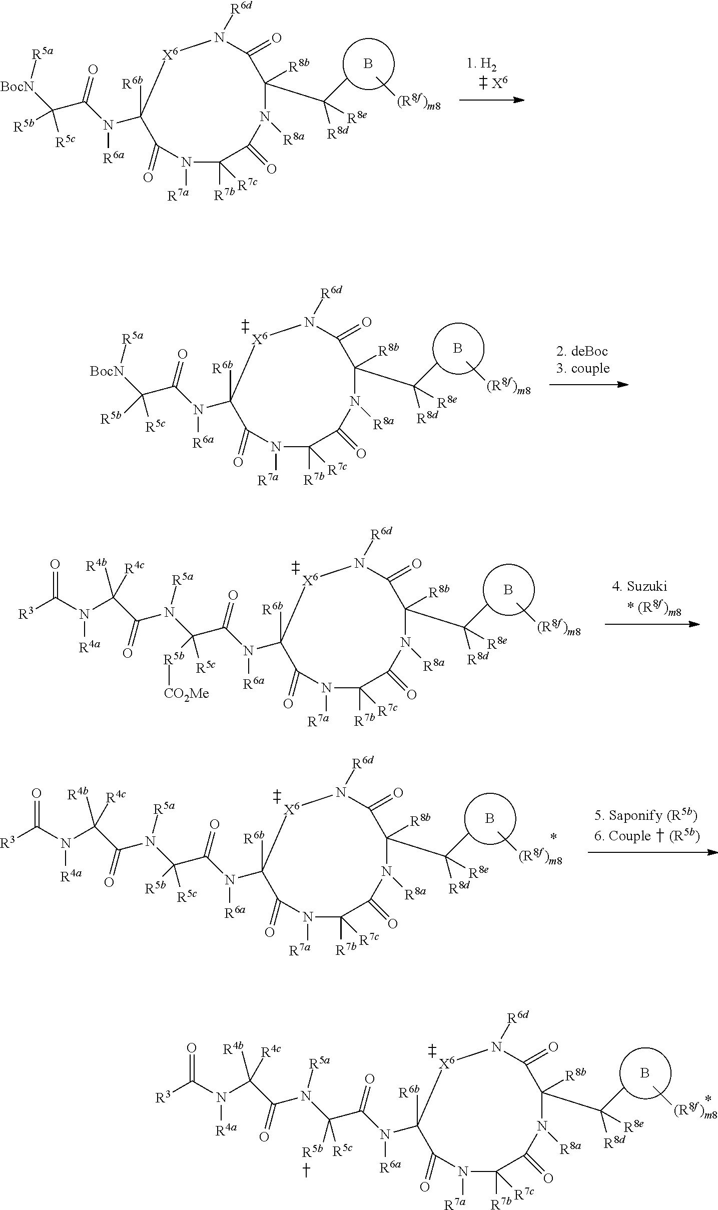

### Representative Procedure for Method 13: Example 419

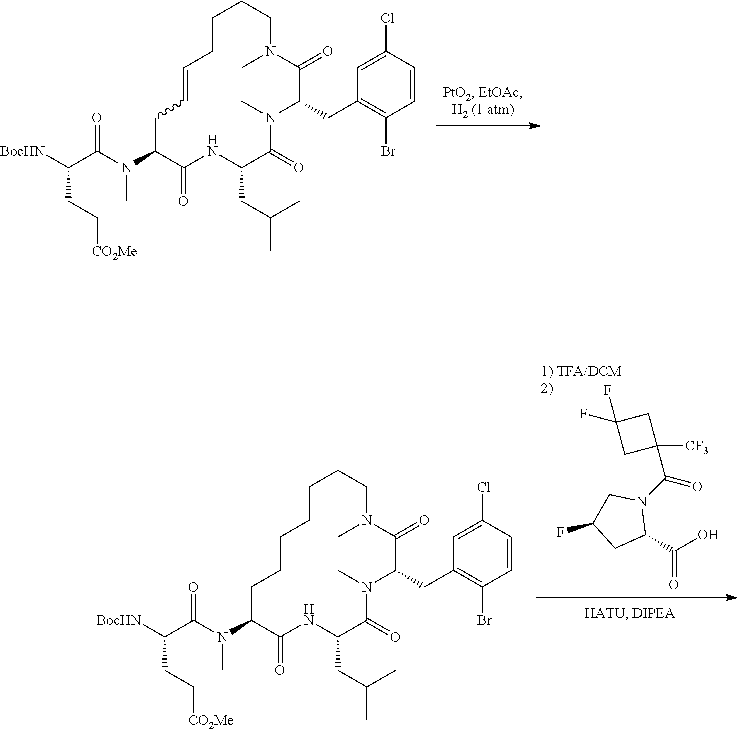 [SMILES: COC(=O)CC[C@H](NC(=O)OC(C)(C)C)C(=O)N(C)[C@H]1CC=CCCCCN(C)C(=O)[C@H](Cc2cc(Cl)ccc2Br)N(C)C(=O)[C@H](CC(C)C)NC1=O ; COC(=O)CC[C@H](NC(=O)OC(C)(C)C)C(=O)N(C)[C@H]1CCCCCCCN(C)C(=O)[C@H](Cc2cc(Cl)ccc2Br)N(C)C(=O)[C@H](CC(C)C)NC1=O ; O=C(O)[C@@H]1C[C@@H](F)CN1C(=O)C1(C(F)(F)F)CC(F)(F)C1]

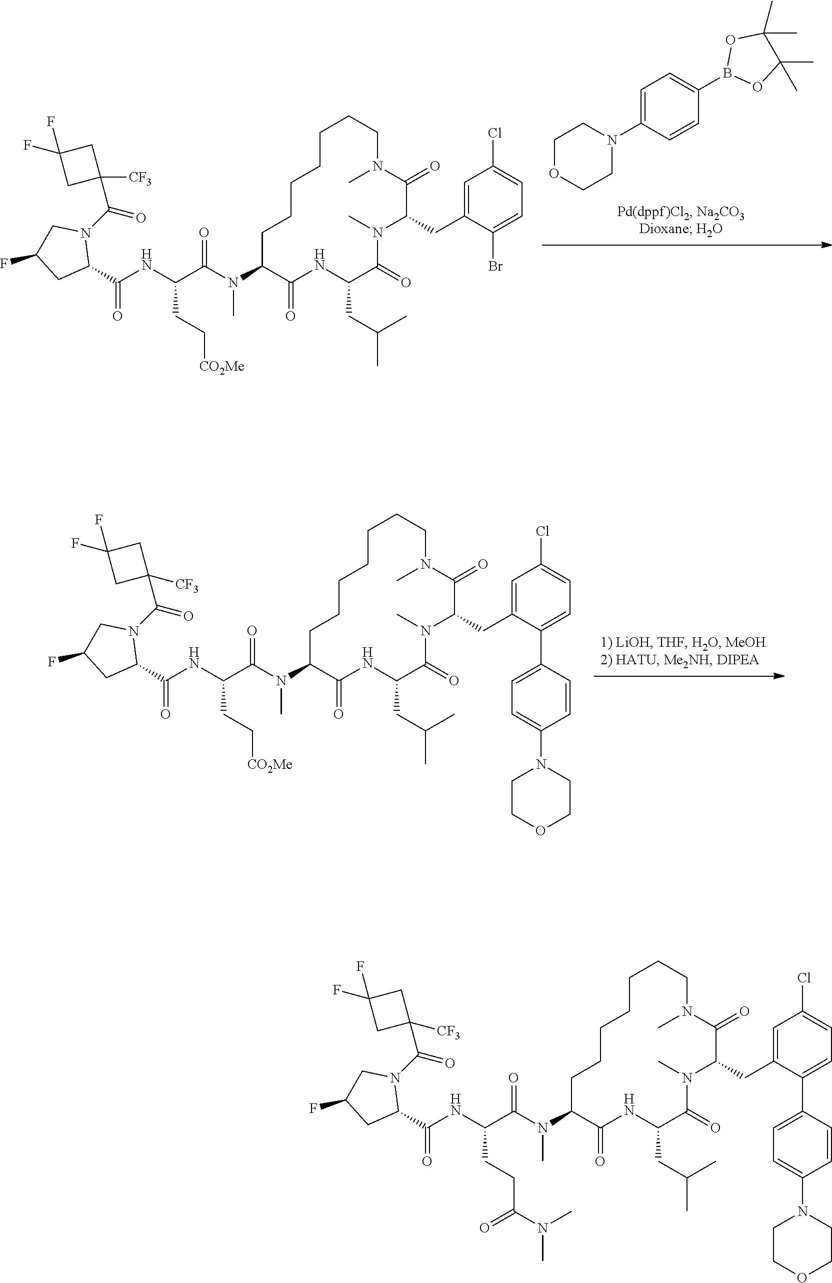

### Representative Experimental for Method 13: Example 419

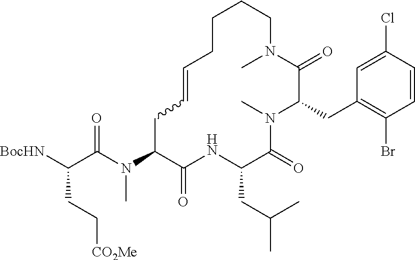 [SMILES: COC(=O)CC[C@H](NC(=O)OC(C)(C)C)C(=O)N(C)[C@H]1CC=CCCCCN(C)C(=O)[C@H](Cc2cc(Cl)ccc2Br)N(C)C(=O)[C@H](CC(C)C)NC1=O]

The starting material for Method 13, 247, Method A with Int. 30, 207. LCMS (ESI): m/z 826.4 (M+H⁺).

### Steps 1-4: ethyl(S)-5-(((3S,6S,9S)-3-((4-chloro-4′-morpholino-[1,1′-biphenyl]-2-yl)methyl)-6-isobutyl-1,4-dimethyl-2,5,8-trioxo-1,4,7-triazacyclohexadecan-9-yl)(methyl)amino)-4-((2S,4R)-1-(3,3-difluoro-1-(trifluoromethyl)cyclobutane-1-carbonyl)-4-fluoropyrrolidine-2-carboxamido)-5-oxopentanoate (248)

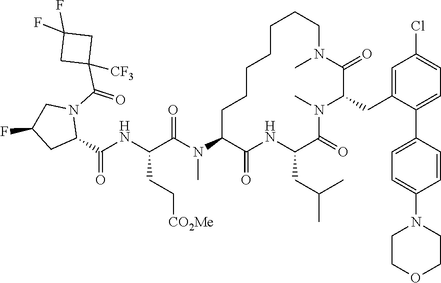 [SMILES: COC(=O)CC[C@H](NC(=O)[C@@H]1C[C@@H](F)CN1C(=O)C1(C(F)(F)F)CC(F)(F)C1)C(=O)N(C)[C@H]1CCCCCCCN(C)C(=O)[C@H](Cc2cc(Cl)ccc2-c2ccc(N3CCOCC3)cc2)N(C)C(=O)[C@H](CC(C)C)NC1=O]

Example 415 was synthesized using identical procedures from Method 7 Steps 1-4 using 247, Int. 117 and 141 as starting materials. LCMS (ESI): m/z 1113.11 (M+H⁺).

### Steps 5-6: (S)—N1-((3S,6S,9S)-3-((4-chloro-4′-morpholino-[1,1′-biphenyl]-2-yl)methyl)-6-isobutyl-1,4-dimethyl-2,5,8-trioxo-1,4,7-triazacyclohexadecan-9-yl)-2-((2S,4R)-1-(3,3-difluoro-1-(trifluoromethyl)cyclobutane-1-carbonyl)-4-fluoropyrrolidine-2-carboxamido)-N1,N5,N5-trimethylpentanediamide (Example 419)

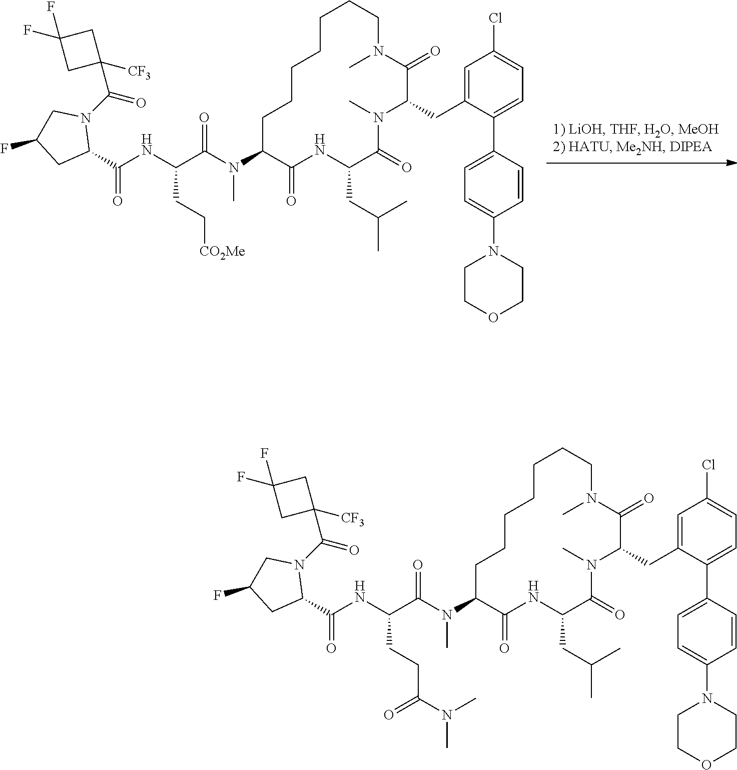 [SMILES: COC(=O)CC[C@H](NC(=O)[C@@H]1C[C@@H](F)CN1C(=O)C1(C(F)(F)F)CC(F)(F)C1)C(=O)N(C)[C@H]1CCCCCCCN(C)C(=O)[C@H](Cc2cc(Cl)ccc2-c2ccc(N3CCOCC3)cc2)N(C)C(=O)[C@H](CC(C)C)NC1=O ; CC(C)C[C@@H]1NC(=O)[C@@H](N(C)C(=O)[C@H](CCC(=O)N(C)C)NC(=O)[C@@H]2C[C@@H](F)CN2C(=O)C2(C(F)(F)F)CC(F)(F)C2)CCCCCCCN(C)C(=O)[C@H](Cc2cc(Cl)ccc2-c2ccc(N3CCOCC3)cc2)N(C)C1=O]

Example 415 (300 mg, 1 Eq, 270 μmol) was dissolved in THE (4.0 mL). In a separate vial, LiOH·water (23.8 mg, 2.1 Eq, 566 μmol) was dissolved in Water (1.0 mL). The LiOH solution was added into the stirring THF solution. MeOH (˜0.1 mL) was added dropwise to ensure a homogeneous reaction mixture. The reaction was quenched with 1N HCl (4 mL) upon the consumption of starting material (monitored by LCMS). The reaction was extracted with EtOAc (10 mL×2). The combined organic layers were washed with brine, dried with MgSO₄, filtered, and concentrated to afford Example 418 (296 mg, 270 mol, 100%) as a white solid. The reaction was carried on without further purification. A small portion (˜15 mg) was purified by RP-HPLC for biological evaluation. LCMS (ESI): m/z 1090.02 (M+H⁺).

Example 418 (35 mg, 1 Eq, 32 μmol) was dissolved in DMF (1.0 mL). HATU (13 mg, 1.1 Eq, 35 μmol) and DIPEA (25 mg, 33 μL, 6 Eq, 0.19 mmol) were subsequently added. The reaction was stirred for 5 min. Then, 1M dimethylamine in THF (14 mg, 0.32 mL, 10 Eq, 0.32 mmol) was added. The reaction was continued until the complete consumption of starting material (monitored by LCMS). The reaction mixture was neutralized to pH ˜4, with 1N HCl filtered, and directly purified by RP-HPLC. Example 419 (20.9 mg, 18.6 μmol, 58%) was isolated as white solid after lyophilization. LCMS (ESI): m/z 1126.12 (M+H⁺).

n. Method 14

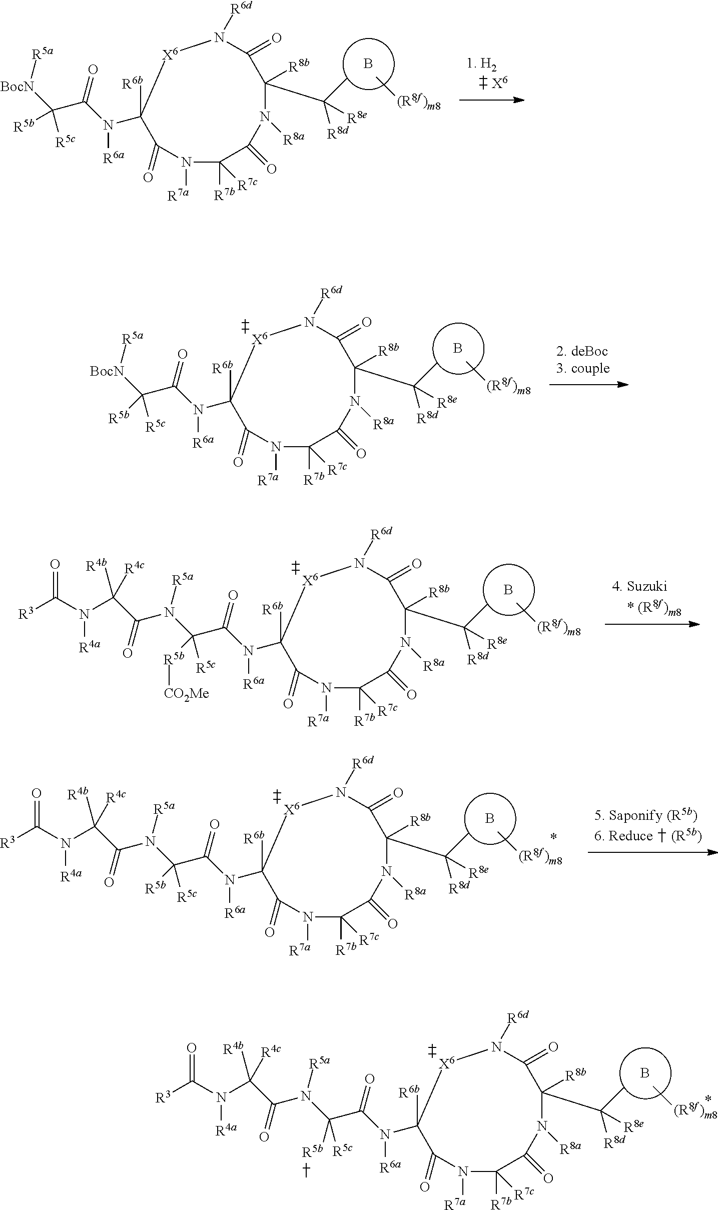

### Representative Procedure for Method 14: Example 420

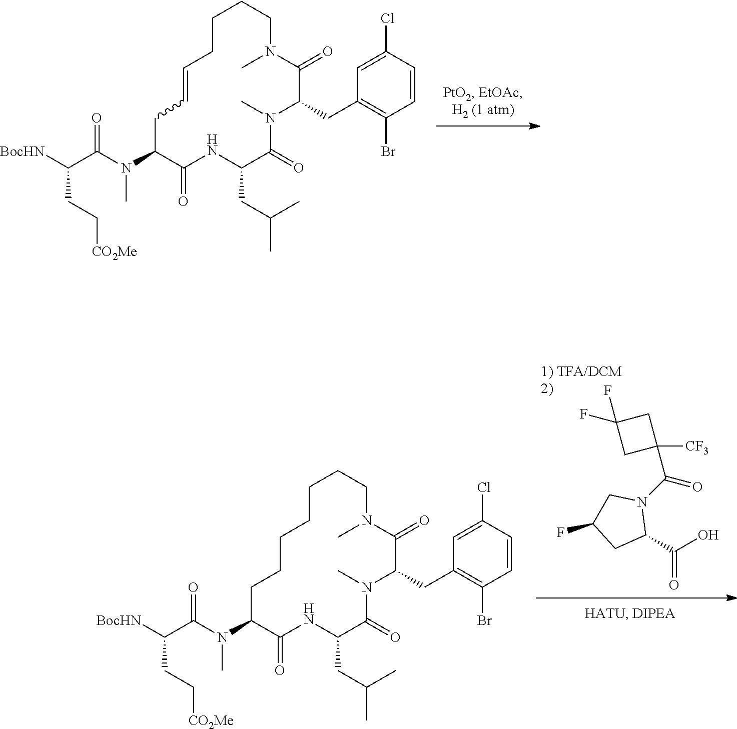 [SMILES: COC(=O)CC[C@H](NC(=O)OC(C)(C)C)C(=O)N(C)[C@H]1CC=CCCCCN(C)C(=O)[C@H](Cc2cc(Cl)ccc2Br)N(C)C(=O)[C@H](CC(C)C)NC1=O ; COC(=O)CC[C@H](NC(=O)OC(C)(C)C)C(=O)N(C)[C@H]1CCCCCCCN(C)C(=O)[C@H](Cc2cc(Cl)ccc2Br)N(C)C(=O)[C@H](CC(C)C)NC1=O ; O=C(O)[C@@H]1C[C@@H](F)CN1C(=O)C1(C(F)(F)F)CC(F)(F)C1]

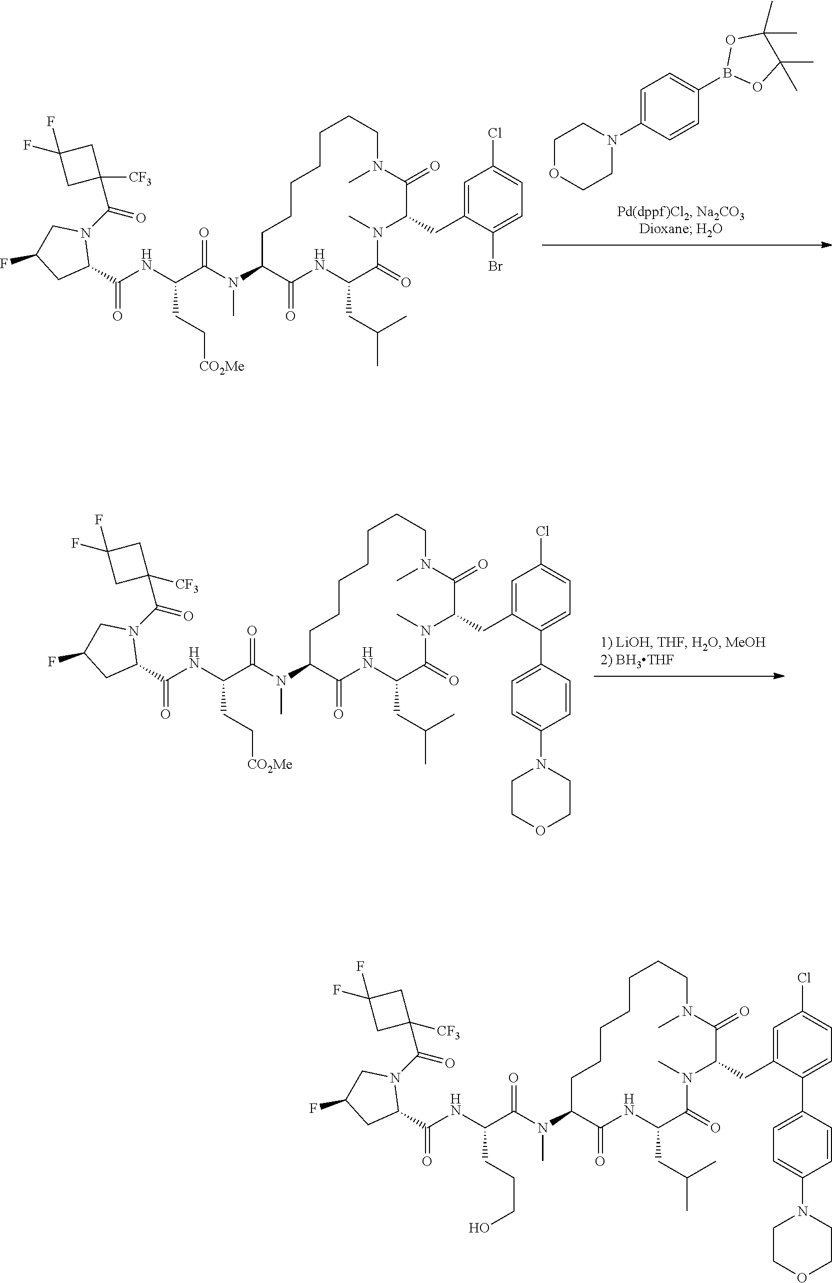

### Representative Experimental for Method 14: Example 420

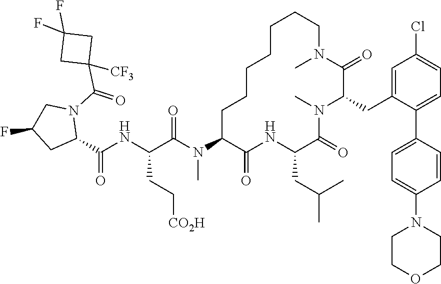 [SMILES: CC(C)C[C@@H]1NC(=O)[C@@H](N(C)C(=O)[C@H](CCC(=O)O)NC(=O)[C@@H]2C[C@@H](F)CN2C(=O)C2(C(F)(F)F)CC(F)(F)C2)CCCCCCCN(C)C(=O)[C@H](Cc2cc(Cl)ccc2-c2ccc(N3CCOCC3)cc2)N(C)C1=O]

The intermediate for Method 14, Example 418, was synthesized using Method 13 steps 1-5.

### Step 6: (2S,4R)—N—((S)-1-(((3S,6S,9S)-3-((4-chloro-4′-morpholino-[1,1′-biphenyl]-2-yl)methyl)-6-isobutyl-1,4-dimethyl-2,5,8-trioxo-1,4,7-triazacyclohexadecan-9-yl)(methyl)amino)-5-hydroxy-1-oxopentan-2-yl)-1-(3,3-difluoro-1-(trifluoromethyl)cyclobutane-1-carbonyl)-4-fluoropyrrolidine-2-carboxamide (Example 420)

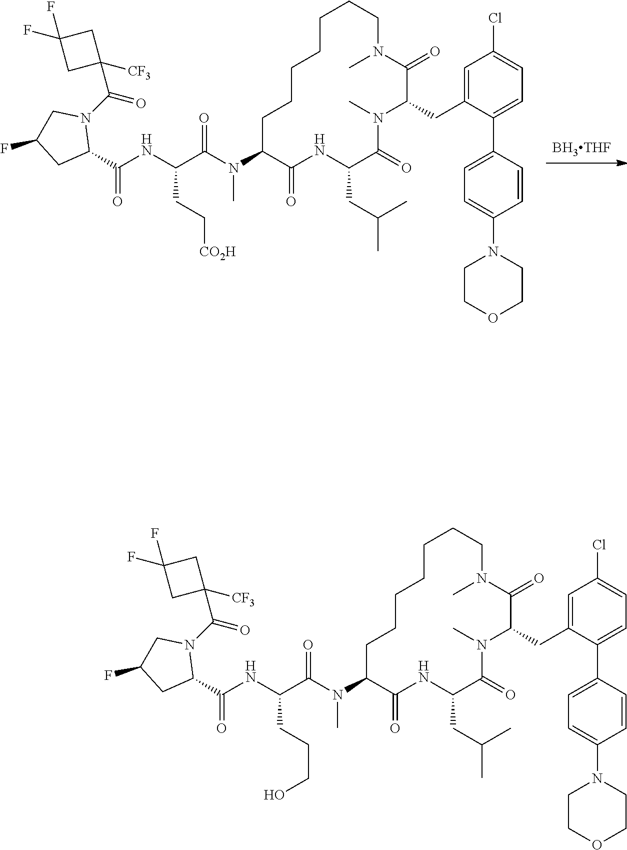

Example 418 (35 mg, 1 Eq, 32 μmol) was dissolved in THF 2 mL and was cooled to 0° C. BH₃·THF (8.2 mg, 96 μL, 1.0 molar in THF, 3 Eq, 96 μmol) was added. The reaction was slowly warmed to RT and stirred overnight. The reaction mixture was quenched with the addition of sat. NaHCO₃, filtered, and directly purified by RP-HPLC. Example 420 (7.8 mg, 7.2 μmol, 23%) was isolated as a white solid after lyophilization. LCMS (ESI): m/z 1085.25 (M+H⁺).

### 3. Exemplary Compounds Preparation Summary Tables

Table 2, below, provides information on the methods used to prepare the exemplified compounds in the current application. This table includes a column listing the Example Number, Macrocyclic Synthesis procedure, and Final Synthesis procedure. Chemical structures for the exemplified compounds are shown in Table 4, while analytical data for these compounds are shown in Table 3.

Certain examples in the following tables include a P1 or a P2 after the example number. This indicates that two isomers of an exemplary compound were isolated using HPLC purification, but the absolute stereochemistry for each isolated isomer was not determined. P1 refers to the first eluting peak, and P2 refers to the second eluting peak.

TABLE 2
Summary Table Procedures for Compound Preparation

| Example | | |
|---|---|---|
| Number | Macrocycle Synthesis | Final Synthesis |
| 1 | Method C | Method 8 with Int. 34 |
| 2 | Method C | Method 8 with Int. 35 |
| 3 | Method C | Method 8 with Int. 33 |
| 4 | Method C | Method 8 with Int. 127 |
| 5-P1 | Method A with Int. 1, Int. 6 | Method 1 with Int. 140 |
| 5-P2 | Method A with Int. 1, Int. 6 | Method 1 with Int. 140 |
| 6-P1 | Method A with Int. 1, Int. 6 | Method 1 |
| 6-P2 | Method A with Int. 1, Int. 6 | Method 1 |
| 7 | Method A with Int. 1, Int. 6 | Method 6 |
| 8 | Method C | Method 8 with Int. 127 |
| 9 | Method A with Int. 1, Int. 6 | Method 6 with Int. 140 |
| 10 | Method A with Int. 1, Int. 6 | Method 6 with Int. 142 |
| 11 | Method A with Int. 6 | Method 1 with Int. 140 |
| 12 | Method C with Int. 3 | Method 8 with Int. 127 |
| 13 | Method C with Int. 3 | Method 8 with Int. 35 |
| 14 | Method A with Int. 6 | Method 1 |
| 15 | Method A with Int. 6 | Method 1 with Int. 143 |
| 16-P1 | Method A with Int. 1, Int. 6 | Method 1 with Int. 143 |
| 16-P2 | Method A with Int. 1, Int. 6 | Method 1 with Int. 143 |
| 17 | Method A with Int. 1, Int. 30 | Method 2 with Int. 46 |
| 18 | Method A with Int. 1, Int. 30 | Method 2 with Int. 47 |
| 19 | Method A with Int. 1, Int. 30 | Method 2 with Int. 48 |
| 20 | Method A with Int. 1, Int. 30 | Method 4 |
| 21 | Method A with Int. 1, Int. 30 | Method 4 with Int. 46 |
| 22 | Method A with Int. 1, Int. 30 | Method 2 with Int. 49 |
| 23 | Method A with Int. 1, Int. 30 | Method 2 with Int. 50 |
| 24 | Method A with Int. 1, Int. 30 | Method 2 with Int. 51 |
| 25 | Method A with Int. 6 | Method 6 |
| 26 | Method A with Int. 1, Int. 30 | Method 2 with Int. 52 |
| 27 | Method A with Int. 1, Int. 30 | Method 2 with Int. 53 |
| 28 | Method A with Int. 1, Int. 30 | Method 2 with Int. 54 |
| 29 | Method A with Int. 30 | Method 2 with Int. 52 |
| 30 | Method A with Int. 30 | Method 2 with Int. 55 |
| 31 | Method A with Int. 30 | Method 2 with Int. 47 |
| 32 | Method A with Int. 1, Int. 30 | Method 2 with Int. 144, Int. 53 |
| 33 | Method A with Int. 1, Int. 30 | Method 2 with Int. 144, Int. 54 |
| 34 | Method A with Int. 30 | Method 2 with Int. 144, Int. 52 |
| 35 | Method A with Int. 30 | Method 2 with Int. 144, Int. 55 |
| 36 | Method A with Int. 30 | Method 2 with Int. 144, Int. 47 |
| 37 | Method A | Method 2 with Int. 52 |
| 38 | Method A | Method 2 with Int. 55 |
| 39 | Method A | Method 2 with Int. 47 |
| 40 | Method A | Method 2 With Int. 144, Int. 52 |
| 41 | Method A | Method 2 with Int. 144, Int. 55 |
| 42 | Method A | Method 2 with Int. 144, Int. 47 |
| 43 | Method A with Int. 1, Int. 30 | Method 2 with Int. 56 |
| 44 | Method A with Int. 30 | Method 2 with Int. 56 |
| 45 | Method A | Method 5 |
| 46 | Method A | Method 2 with Int. 57 |
| 47 | Method A | Method 2 with Int. 51 |
| 48 | Method A with Int. 6 | Method 1 with Int. 144 |
| 49 | Method A with Int. 30 | Method 4 with Int. 52 |
| 50 | Method A with Int. 30 | Method 4 |
| 51 | Method A with Int. 30 | Method 4 with Int. 46 |
| 52 | Method A with Int. 30 | Method 5 |
| 53 | Method A with Int. 1, Int. 30 | Method 3 with Int. 67, Int. 36 |
| 54 | Method A with Int. 1, Int. 6, Int. 32 | Method 1 |
| 55 | Method A with Int. 1, Int. 6, Int. 32 | Method 6 |
| 56-P1 | Method A with Int. 1, Int. 30 | Method 2 with Int. 58 |
| 56-P2 | Method A with Int. 1, Int. 30 | Method 2 with Int. 58 |
| 57-P1 | Method A with Int. 30 | Method 7 with Int. 134 |
| 57-P2 | Method A with Int. 30 | Method 7 with Int. 134 |
| 58 | Method A | Method 7 with Int. 134 |
| 59-P1 | Method A | Method 5 With Int. 45 |
| 59-P2 | Method A | Method 5 With Int. 45 |
| 60 | Method A with Int. 30 | Method 7 with Int. 137 |
| 61 | Method A with Int. 1, Int. 30 | Method 4 with Int. 68 |
| 62 | Method A with Int. 1, Int. 30 | Method 4 with Int. 69 |
| 63 | Method A with Int. 1, Int. 30 | Method 5 with Int. 68 |
| 64 | Method A with Int. 1, Int. 30 | Method 5 with Int. 69 |
| 65 | Method A | Method 4 with Int. 52 |
| 66 | Method A with Int. 1, Int. 30 | Method 4 with Int. 52 |
| 67 | Method A with Int. 1, Int. 30 | Method 4 with Int. 59 |
| 68 | Method A | Method 4 with Int. 59 |
| 69 | Method A | Method 4 with Int. 144, Int. 52 |
| 70 | Method A with Int. 1, Int. 30 | Method 4 with Int. 144, Int. 52 |
| 71 | Method A with Int. 4, Int. 30 | Method 4 with Int. 46 |
| 72 | Method A with Int. 4, Int. 30 | Method 4 with Int. 52 |
| 73 | Method A with Int. 4, Int. 30 | Method 4 with Int. 59 |
| 74 | Method A with Int. 4, Int. 30 | Method 4 |
| 75 | Method A with Int. 30 | Method 4 with Int. 70 |
| 76 | Method A with Int. 30 | Method 4 with Int. 145, Int. 52 |
| 77 | Method A with Int. 30 | Method 4 with Int. 146, Int. 52 |
| 78 | Method A with Int. 30 | Method 2 with Int. 59 |
| 79 | Method A with Int. 30 | Method 2 with Int. 60 |
| 80 | Method A with Int. 30 | Method 2 with Int. 144, Int. 60 |
| 81 | Method A with Int. 30 | Method 2 with Int. 144, Int. 59 |
| 82 | Method A with Int. 30 | Method 4 with Int. 147, Int. 52 |
| 83 | Method A | Method 2 with Int. 144, Int. 61 |
| 84 | Method A | Method 2 with Int. 62 |
| 85 | Method A | Method 2 with Int. 63 |
| 86 | Method A | Method 2 with Int. 59 |
| 87 | Method A | Method 2 with Int. 64 |
| 88 | Method A | Method 2 with Int. 65 |
| 89 | Method A with Int. 1, Int. 30 | Method 2 with Int. 66 |
| 90 | Method A with Int. 1, Int. 30 | Method 3 with Int. 67, Int. 37 |
| 91 | Method A with Int. 1, Int. 30 | Method 3 with Int. 67, Int. 38 |
| 92 | Method A | Method 4 with Int. 144, Int. 61 |
| 93 | Method A | Method 4 with Int. 62 |
| 94 | Method A | Method 4 with Int. 63 |
| 95 | Method A | Method 4 with Int. 64 |
| 96 | Method A | Method 4 with Int. 65 |
| 97 | Method A with Int. 1, Int. 30 | Method 4 with Int. 66 |
| 98 | Method A with Int. 1, Int. 30 | Method 7 with Int. 138 |
| 99 | Method A with Int. 1, Int. 30 | Method 7 with Int. 138 |
| 100 | Method A | Method 4 with Int. 50 |
| 101 | Method A | Method 5 with Int. 66 |
| 102-P1 | Method A with Int. 1, Int. 7 | Method 1 |
| 102-P2 | Method A with Int. 1, Int. 7 | Method 1 |
| 103 | Method A with Int. 1, Int. 7 | Method 6 |
| 104 | Method A with Int. 7 | Method 1 |
| 105 | Method A with Int. 7 | Method 6 |
| 106 | Method A with Int. 7 | Method 1 with Int. 144 |
| 107-P1 | Method A with Int. 1, Int. 7 | Method 1 with Int. 144 |
| 107-P2 | Method A with Int. 1, Int. 7 | Method 1 with Int. 144 |
| 108 | Method A with Int. 1, Int. 7 | Method 6 with Int. 144 |
| 109 | Method A with Int. 7 | Method 6 with Int. 144 |
| 110 | Method A with Int. 30 | Method 4 with Int. 59 |
| 111 | Method A with Int. 30 | Method 4 with Int. 71 |
| 112 | Method A with Int. 30 | Method 4 with Int. 65 |
| 113 | Method A with Int. 30 | Method 4 with Int. 63 |
| 114 | Method A with Int. 30 | Method 4 with Int. 62 |
| 115 | Method A with Int. 8 | Method 1 with Int. 144 |
| 116 | Method A with Int. 8 | Method 1 |
| 117-P1 | Method A with Int. 1, Int. 8 | Method 1 with Int. 144 |
| 117-P2 | Method A with Int. 1, Int. 8 | Method 1 with Int. 144 |
| 118-P1 | Method A with Int. 1, Int. 8 | Method 1 |
| 118-P2 | Method A with Int. 1, Int. 8 | Method 1 |
| 119 | Method A with Int. 10 | Method 6 with Int. 144 |
| 120 | Method A with Int. 10 | Method 6 |
| 121 | Method A | Method 4 with Int. 72 |
| 122 | Method A | Method 4 with Int. 147, Int. 59 |
| 123 | Method A | Method 4 with Int. 130 |
| 124 | Method A | Method 7 with Int. 135 |
| 125 | Method A with Int. 1, Int. 9 | Method 1 |
| 126 | Method A with Int. 9 | Method 1 |
| 127 | Method A with Int. 1, Int. 9 | Method 6 |
| 128 | Method A with Int. 9 | Method 6 |
| 129 | Method A with Int. 1, Int. 10 | Method 1 |
| 130 | Method A with Int. 10 | Method 1 |
| 131 | Method A with Int. 1, Int. 10 | Method 6 |
| 132 | Method A with Int. 1, Int. 9 | Method 1 with Int. 144 |
| 133 | Method A with Int. 9 | Method 1 with Int. 144 |
| 134 | Method A with Int. 1, Int. 9 | Method 6 with Int. 144 |
| 135 | Method A with Int. 9 | Method 6 with Int. 144 |
| 136-P1 | Method A with Int. 1, Int. 10 | Method 1 with Int. 144 |
| 136-P2 | Method A with Int. 1, Int. 10 | Method 1 with Int. 144 |
| 137 | Method A with Int. 10 | Method 1 with Int. 144 |
| 138 | Method A with Int. 1, Int. 10 | Method 6 with Int. 144 |
| 139 | Method A with Int. 12 | Method 6 with Int. 144 |
| 140 | Method A with Int. 12 | Method 6 |
| 141 | Method A with Int. 1, Int. 12 | Method 6 with Int. 144 |
| 142 | Method A with Int. 1, Int. 12 | Method 6 |
| 143 | Method A with Int. 30 | Method 7 with Int. 138, Int. 37 |
| 144 | Method A with Int. 30 | Method 7 with Int. 138, Int. 38 |
| 145 | Method A with Int. 30 | Method 7 with Int. 138, Int. 36 |
| 146 | Method A | Method 4 with Int. 148, Int. 59 |
| 147 | Method A | Method 4 with Int. 149, Int. 59 |
| 148 | Method A | Method 4 with Int. 73 |
| 149 | Method A | Method 7 with Int. 134 |
| 150 | Method A | Method 7 with Int. 134 |
| 151 | Method A | Method 7 with Int. 135 |
| 152 | Method A with Int. 1, Int. 11 | Method 1 |
| 153 | Method A with Int. 11 | Method 1 |
| 154 | Method A with Int. 1, Int. 11 | Method 6 |
| 155 | Method A with Int. 11 | Method 6 |
| 156-P1 | Method A with Int. 1, Int. 11 | Method 1 with Int. 144 |
| 156-P2 | Method A with Int. 1, Int. 11 | Method 1 with Int. 144 |
| 157 | Method A with Int. 11 | Method 1 with Int. 144 |
| 158 | Method A with Int. 1, Int. 11 | Method 6 with Int. 144 |
| 159 | Method A with Int. 11 | Method 6 with Int. 144 |
| 160 | Method A with Int. 1, Int. 8 | Method 6 |
| 161 | Method A with Int. 1, Int. 8 | Method 6 with Int. 144 |
| 162 | Method A with Int. 8 | Method 6 |
| 163 | Method A with Int. 8 | Method 6 with Int. 144 |
| 164 | Method A | Method 4 with Int. 74 |
| 165 | Method A | Method 7 |
| 166 | Method A | Method 7 with Int. 135 |
| 167 | Method A with Int. 30 | Method 4 with Int. 75 |
| 168 | Method B with Int. 30 | Method 2 with Int. 52 |
| 169 | Method A with Int. 30 | Method 4 with Int. 131 |
| 170 | Method A with Int. 30 | Method 4 with Int. 76 |
| 171 | Method A with Int. 30 | Method 4 with Int. 77 |
| 172 | Method A with Int. 30 | Method 4 with Int. 78 |
| 173 | Method B with Int. 30 | Method 2 with Int. 144, Int. 52 |
| 174 | Method B with Int. 30 | Method 2 with Int. 149, Int. 52 |
| 175 | Method B with Int. 30 | Method 2 with Int. 59 |
| 176 | Method B with Int. 30 | Method 2 with Int. 144, Int. 59 |
| 177 | Method A with Int. 30 | Method 4 with Int. 79 |
| 178 | Method A with Int. 30 | Method 4 with Int. 80 |
| 179 | Method A with Int. 30 | Method 4 with Int. 81 |
| 180 | Method A with Int. 30 | Method 4 with Int. 132 |
| 181 | Method A with Int. 30 | Method 4 with Int. 82 |
| 182 | Method A with Int. 30 | Method 4 with Int. 83 |
| 183 | Method A with Int. 30 | Method 4 with Int. 148, Int. 52 |
| 184 | Method A with Int. 30 | Method 4 with Int. 149, Int. 52 |
| 185 | Method A with Int. 30 | Method 4 with Int. 144, Int. 52 |
| 186 | Method A | Method 4 with Int. 144, Int. 59 |
| 187 | Method A with Int. 30 | Method 4 with Int. 84 |
| 188 | Method A with Int. 1, Int. 30 | Method 4 with Int. 144, Int. 59 |
| 189 | Method A with Int. 1, Int. 30 | Method 2 with Int. 59 |
| 190 | Method A with Int. 30 | Method 4 with Int. 73 |
| 191 | Method A with Int. 30 | Method 4 with Int. 85 |
| 192 | Method A with Int. 30 | Method 4 with Int. 86 |
| 193 | Method A with Int. 1, Int. 30 | Method 7 with Int. 134 |
| 194 | Method A with Int. 1, Int. 30 | Method 7 with Int. 135 |
| 195 | Method A with Int. 1 | Method 2 with Int. 52 |
| 196 | Method A with Int. 30 | Method 4 with Int. 121 |
| 197 | Method A with Int. 30 | Method 4 with Int. 87 |
| 198 | Method A with Int. 30 | Method 4 with Int. 88 |
| 199 | Method A with Int. 30 | Method 4 with Int. 87 |
| 200 | Method A with Int. 30 | Method 4 with Int. 90 |
| 201 | Method A with Int. 30 | Method 4 with Int. 91 |
| 202 | Method A with Int. 30 | Method 4 with Int. 92 |
| 203 | Method A with Int. 30 | Method 4 with Int. 143, Int. 52 |
| 204 | Method A with Int. 30 | Method 4 with Int. 150, Int. 52 |
| 205 | Method A with Int. 30 | Method 4 with Int. 93 |
| 206 | Method A with Int. 30 | Method 4 with Int. 94 |
| 207 | Method A with Int. 30 | Method 4 with Int. 95 |
| 208 | Method A with Int. 30 | Method 4 with Int. 96 |
| 209 | Method A with Int. 30 | Method 4 with Int. 97 |
| 210 | Method A with Int. 30 | Method 4 with Int. 98 |
| 211 | Method A with Int. 30 | Method 4 with Int. 99 |
| 212 | Method A with Int. 30 | Method 4 with Int. 100 |
| 213 | Method A with Int. 1 | Method 4 with Int. 59 |
| 214 | Method A with Int. 1 | Method 4 with Int. 73 |
| 215 | Method A with Int. 1 | Method 4 with Int. 52 |
| 216 | Method A with Int. 30 | Method 4 with Int. 101 |
| 217 | Method A with Int. 1 | Method 4 |
| 218 | Method A with Int. 30 | Method 4 with Int. 52 |
| 219 | Method A with Int. 7, Int. 41 | Method 6 |
| 220 | Method A with Int. 7, Int. 41 | Method 6 with Int. 144 |
| 221 | Method A with Int. 7, Int. 41 | Method 6 with Int. 149 |
| 222 | Method A with Int. 13 | Method 6 |
| 223 | Method A with Int. 13 | Method 6 with Int. 149 |
| 224 | Method A with Int. 13 | Method 6 with Int. 144 |
| 225 | Method A with Int. 14 | Method 6 |
| 226 | Method A with Int. 14 | Method 6 with Int. 149 |
| 227 | Method A with Int. 14 | Method 6 with Int. 144 |
| 228 | Method A with Int. 15 | Method 6 |
| 229 | Method A with Int. 15 | Method 6 with Int. 149 |
| 230 | Method A with Int. 15 | Method 6 with Int. 144 |
| 231 | Method A with Int. 30 | Method 4 with Int. 102 |
| 232 | Method A with Int. 30 | Method 4 with Int. 103 |
| 233 | Method A with Int. 30 | Method 4 with Int. 85 |
| 234 | Method A with Int. 30 | Method 4 with Int. 104 |
| 235 | Method A with Int. 30 | Method 4 with Int. 105 |
| 236 | Method A with Int. 30 | Method 4 with Int. 106 |
| 237 | Method A with Int. 30 | Method 4 with Int. 107 |
| 238 | Method A with Int. 30 | Method 4 with Int. 64 |
| 239 | Method A with Int. 30 | Method 4 with Int. 108 |
| 240 | Method A with Int. 16 | Method 6 |
| 241 | Method A with Int. 1 | Method 7 with Int. 135 |
| 242 | Method A with Int. 30 | Method 4 with Int. 74 |
| 243 | Method A with Int. 17 | Method 6 |
| 244 | Method A with Int. 18 | Method 6 |
| 245 | Method A with Int. 18 | Method 6 with Int. 144 |
| 246 | Method A with Int. 19 | Method 6 |
| 247 | Method A with Int. 19 | Method 6 with Int. 144 |
| 248 | Method A with Int. 19 | Method 6 with Int. 148 |
| 249 | Method A with Int. 30 | Method 4 with Int. 109 |
| 250 | Method A with Int. 30 | Method 4 with Int. 110 |
| 251 | Method A with Int. 30 | Method 4 with Int. 111 |
| 252 | Method A | Method 4 with Int. 112 |
| 253 | Method A with Int. 30 | Method 4 with Int. 113 |
| 254 | Method A with Int. 30 | Method 4 with Int. 112 |
| 255 | Method A with Int. 30 | Method 4 with Int. 114 |
| 256 | Method A with Int. 30 | Method 4 with Int. 115 |
| 257 | Method A with Int. 30 | Method 4 with Int. 116 |
| 258 | Method B | Method 1 |
| 259 | Method A with Int. 20 | Method 6 |
| 260 | Method A with Int. 20 | Method 6 with Int. 148 |
| 261 | Method A with Int. 30 | Method 4 with Int. 117 |
| 262 | Method A with Int. 30 | Method 4 with Int. 118 |
| 263 | Method A with Int. 30 | Method 4 with Int. 130 |
| 264 | Method A with Int. 1 | Method 4 with Int. 130 |
| 265 | Method A with Int. 30 | Method 4 with Int. 119 |
| 266 | Method A with Int. 30 | Method 4 with Int. 120 |
| 267 | Method A with Int. 2, Int. 30, Int. 42 | Method 2 with Int. 47 |
| 268 | Method A with Int. 13 | Method 6 with Int. 150 |
| 269 | Method A with Int. 14 | Method 6 with Int. 150 |
| 270 | Method A with Int. 1, Int. 30 | Method 4 with Int.130 |
| 27 | Method A | Method 4 with Int. 121 |
| 272 | Method A with Int. 30 | Method 4 with Int. 89 |
| 273 | Method A with Int. 2, Int. 30, Int. 42 | Method 2 with Int. 64 |
| 274 | Method A with Int. 1, Int. 13 | Method 6 with Int. 148 |
| 275 | Method A with Int. 1, Int. 13 | Method 6 |
| 276 | Method A with Int. 1, Int. 30 | Method 4 with Int. 133 |
| 277 | Method A with Int. 1, Int. 30 | Method 4 with Int. 122 |
| 278 | Method B with Int. 31 | Method 2 with Int. 64 |
| 279 | Method A | Method 4 with Int. 123 |
| 280 | Method A with Int. 1 | Method 4 with Int. 133 |
| 281 | Method A with Int. 1 | Method 4 with Int. 112 |
| 282 | Method A | Method 4 with Int. 112, Int. 39 |
| 283 | Method A with Int. 30 | Method 4 with Int. 112, Int. 39 |
| 284 | Method A with Int. 1, Int. 30 | Method 4 with Int. 50 |
| 285 | Method A with Int. 5, Int. 30, Int. 43 | Method 2 with Int. 52 |
| 286 | Method A with Int. 5, Int. 30, Int. 43 | Method 2 with Int. 47 |
| 287 | Method A with Int. 5, Int. 30, Int. 43 | Method 2 with Int. 59 |
| 288 | Method A with Int. 1, Int. 30 | Method 4 with Int. 40 |
| 289 | Method A with Int. 30 | Method 4 with Int. 40 |
| 290 | Method A with Int. 1 | Method 4 with Int. 123 |
| 291 | Method A with Int. 1, Int. 30 | Method 4 with Int. 123 |
| 292 | Method A with Int. 30 | Method 4 with Int. 123 |
| 293 | Method A | Method 4 with Int. 124 |
| 294 | Method A with Int. 1 | Method 4 with Int. 124 |
| 295 | Method A with Int. 1, Int. 30 | Method 4 with Int. 124 |
| 296 | Method A with Int. 30 | Method 4 with Int. 124 |
| 297 | Method A with Int. 30 | Method 4 with Int. 125 |
| 298 | Method B with Int. 30 | Method 2 with Int. 64 |
| 299 | Method A with Int. 30 | Method 4 with Int. 148, Int. 64 |
| 300 | Method A with Int. 1, Int. 30 | Method 4 with Int. 112 |
| 301 | Method A with Int. 30 | Method 4 with Int. 150, Int. 64 |
| 302 | Method A with Int. 1, Int. 22 | Method 6 |
| 303 | Method A | Method 4 with Int. 148, Int. 64 |
| 304 | Method A | Method 4 with Int. 150, Int. 64 |
| 305 | Method A with Int. 30 | Method 4 with Int. 126 |
| 306 | Method A | Method 4 with Int. 64 |
| 307 | Method A with Int. 30 | Method 4 with Int. 64 |
| 308 | Method A with Int. 30 | Method 7 with Int. 128 |
| 309 | Method A with Int. 30 | Method 4 with Int. 44 |
| 310 | Method A | Method 4 with Int. 126 |
| 311 | Method A with Int. 1, Int. 23 | Method 6 |
| 312 | Method A with Int. 1, Int. 30 | Method 7 with Int. 134 |
| 313 | Method A with Int. 1, Int. 30 | Method 7 with Int. 134 |
| 314 | Method A with Int. 23 | Method 6 |
| 315 | Method A with Int. 1 | Method 7 with Int. 134 |
| 316 | Method A with Int. 1 | Method 7 with Int. 134 |
| 317 | Method A | Method 7 with Int. 134 |
| 318 | Method A | Method 7 with Int. 134 |
| 319 | Method A with Int. 24 | Method 6 |
| 320 | Method A with Int. 25 | Method 6 |
| 321 | Method A with Int. 26 | Method 6 |
| 322 | Method A with Int. 27 | Method 6 |
| 323 | Method A with Int. 28 | Method 6 |
| 324 | Method A with Int. 29 | Method 6 |
| 325 | Method A with Int. 1 | Method 7 with Int. 135 |
| 326 | Method A | Method 7 with Int. 135 |
| 327 | Method A with Int. 1, Int. 30 | Method 7 with Int. 135 |
| 328 | Method A | Method 7 with Int. 135 |
| 329 | Method A | Method 7 with Int. 135 |
| 330 | Method A with Int. 1, Int. 26 | Method 6 |
| 331 | Method A with Int. 1, Int. 27 | Method 6 |
| 332 | Method A with Int. 28 | Method 6 with Int. 148 |
| 333 | Method A with Int. 1, Int. 30 | Method 4 with Int. 117 |
| 334 | Method A with Int. 1 | Method 4 with Int. 117 |
| 335 | Method A with Int. 30 | Method 4 with Int. 117, Int. 150 |
| 336 | Method A with Int. 30 | Method 4 with Int. 157 |
| 337 | Method A | Method 9 with Int. 117 |
| 338 | Method A with Int. 30 | Method 7 (Steps 1-4) with Int. 161 |
| 339 | Method A with Int. 30 | Method 9 with Int. 164 |
| 340 | Method A with Int. 30 | Method 7 (Steps 1-4) with Int. 167 |
| 341 | Method A with Int. 30 | Method 4 with Int. 168 |
| 342 | Method A with Int. 30 | Method 9 with Int. 165 |
| 343 | Method A with Int. 30 | Method 4 with Int. 52, Int. 162 |
| 344 | Method A with Int. 30 | Method 9 with Int. 163 |
| 345 | Method A with Int. 30 | Method 9 with Int. 166 |
| 346 | Method A with Int. 30 | Method 7 (Steps 1-4) with Int. 199 |
| 347 | Method A with Int. 30 | Method 7 (Steps 1-4) with Int. 169 |
| 348 | Method A with Int. 30 | Method 7 (Steps 1-4) with Int. 171 |
| 349 | Method A with Int. 30 | Method 9 with Int. 173 |
| 350 | Method A with Int. 30 | Method 4 with Int. 117, Int. 174 |
| 351 | Method A with Int. 30 | Method 9 with Int. 177 |
| 352 | Method A with Int. 30 | Method 7 with Int. 404 |
| 353 | Method A | Method 9 with Int. 159 |
| 354 | Method A with Int. 30 | Method 9 with Int. 160 |
| 355 | Method A with Int. 30 | Method 7 (Steps 1-4) with Int. 172 |
| 356 | Method A with Int. 30 | Method 9 with Int. 403 |
| 357 | Method A with Int. 30 | Method 9 with Int. 179 |
| 358 | Method A with Int. 30 | Method 9 with Int. 170 |
| 359 | Method A with Int. 30 | Method 9 with Int. 155 |
| 360 | Method A with Int. 30 | Method 9 (Steps 1-4) with Int. 156 |
| 361 | Method A with Int. 30 | Method 9 with Int. 402 |
| 362 | Method A with Int. 30 | Method 9 with Int. 178 |
| 363 | Method A with Int. 30 | Method 7 (Steps 1-4) with Int. 400 |
| 364 | Method A with Int. 30 | Method 7 (Steps 1-4) with Int. 401 |
| 365 | Method A with Int. 30 | Method 4 with Int. 117, Int. 180 |
| 366 | Method A with Int. 30 | Method 9 with Int. 181 |
| 367 | Method A with Int. 30 | Method 9 with Int. 182 |
| 368 | Method A with Int. 30 | Method 7 (Steps 1-4) with Int. 183 |
| 369 | Method A with Int. 30 | Method 7 (Steps 1-4) with Int. 176 |
| 370 | Method A with Int. 30 | Method 7 (Steps 1-4) with Int. 186 |
| 371 | Method A with Int. 30 | Method 7 (Steps 1-4) with Int. 175 |
| 372 | Method A with Int. 30 | Method 7 with Int. 405 |
| 373 | Method A with Int. 30 | Method 7 (Steps 1-4) with Int. 184 |
| 374 | Method A with Int. 30 | Method 7 (Steps 1-4) with Int. 185 |
| 375 | Method A with Int. 30 | Method 7 (Steps 1-4) with Int. 158 |
| 376 | Method A with Int. 30 | Method 7 (Steps 1-4) with Int. 188 |
| 377 | Method A with Int. 30 | Method 4 with Int. 117, Int. 189 |
| 378 | Method A with Int. 30 | Method 7 (Steps 1-4) with Int. 190 |
| 379 | Method A with Int. 30 | Method 7 (Steps 1-4) with Int. 187 |
| 380 | Method A with Int. 30 | Method 7 (Steps 1-4) with Int. 191 |
| 381 | Method A with Int. 30 | Method 7 (Steps 1-4) with Int. 194 |
| 382 | Method A with Int. 30 | Method 10 |
| 383 | Method A with Int. 30 | Method 11 |
| 384 | Method A with Int. 30 | Method 7 (Steps 1-4) with Int. 196 |
| 385 | Method A with Int. 30 | Method 7 (Steps 1-4) with Int. 197 |
| 386 | Method A with Int. 30 | Method 7 (Steps 1-4) with Int. 198 |
| 387 | Method A with Int. 30, Int. 195 | Method 7 (Steps 1-4) with Int. 195, Int. 157 |
| 388 | Method A with Int. 30, Int. 41 | Method 7 (Steps 1-4) with Int. 157 |
| 389 | Method A with Int. 30 | Method 7 (Steps 1-4) with Int. 192 |
| 390 | Method A with Int. 30 | Method 9 with Int. 209 |
| 391 | Method A | Method 9 with Int. 210 |
| 392 | Method A with int. 206 | Method 12 with Int. 211 |
| 393 | Method A with int. 206 | Method 12 with Int. 212 |
| 394 | Method A with int. 206 | Method 12 with Int. 213 |
| 395 | Method A with Int. 1, 30 | Method 9 with Int. 172 |
| 396 | Method A with Int. 30 | Method 9 with Int. 214 |
| 397 | Method B with Int. 3, 30 | Method 9 with Int. 159 |
| 398 | Method A with Int. 30 | Method 4 with Int. 117, Int. 215 |
| 399 | Method A with Int. 30 | Method 4 with Int. 117, Int. 215 |
| 400 | Method A with Int. 30 | Method 4 with Int. 117, Int. 217 |
| 401 | Method A | Method 9 with Int. 218 |
| 402 | Method A with Int. 30 | Method 9 with Int. 219 |
| 403 | Method A with Int. 30 | Method 9 With Int. 220 |
| 404 | Method A with Int. 30, 195 | Method 4 with Int. 117 |
| 405 | Method D | Method 4 with Int. 159 |
| 406 | Method A with Int. 30 | Method 9 with Int. 221 |
| 407 | Method A with Int. 30 | Method 9 with Int. 222 |
| 408 | Method A with Int. 30 | Method 9 with Int. 223 |
| 409 | Method A with Int. 30 | Method 9 with Int. 225 |
| 410 | Method A with Int. 30 | Method 9 with Int. 226 |
| 411 | Method A with Int. 30 | Method 9 with Int. 227 |
| 412 | Method A with Int. 30 | Method 9 with Int. 228 |
| 413 | Method A with Int. 30 | Method 9 with Int. 229 |
| 414 | Method A with Int. 30 | Method 9 with int. 230 |
| 415 | Method A with Int. 30, 207 | Method 4 with int. 117 |
| 416 | Method A with Int. 30, 207 | Method 12 with Int. 236 |
| 417 | Method A with Int. 30 | Method 4 with Int. 231 |
| 418 | Method A with Int. 30, 207 | Method 13 steps 1-5 |
| 419 | Method A with Int. 30, 207 | Method 13 |
| 420 | Method A with Int. 30, 207 | Method 13 with Int. 237 |
| 421 | Method A with Int. 30, 207 | Method 14 |
| 422 | Method A with Int. 30, 208 | Method 4 with Int. 159 |
| 423 | Method A with int. 206 | Method 12 with Int. 232 |
| 424 | Method A with int. 206 | Method 12 with Int. 233 |
| 425 | Method A with int. 206 | Method 12 with Int. 234 |
| 426 | Method A with int. 206 | Method 12 with Int. 235 |

Table 3, below, provides the expected (Exact Mass) and observed molecular weight for each exemplary compound listed in Table 2.

TABLE 3
Analytical Data for Exemplary Compounds of Table 2

| Ex. Number | Exact Mass | Observed m/z |
|---|---|---|
| 1 | 802.32 | 803.33 |
| 2 | 846.33 | 847.49 |
| 3 | 818.29 | 819.44 |
| 4 | 908.32 | 909.43 |
| 5-P1 | 872.34 | 873.46 |
| 5-P2 | 872.34 | 873.46 |
| 6-P1 | 922.34 | 923.42 |
| 6-P2 | 922.34 | 923.42 |
| 7 | 924.35 | 925.48 |
| 8 | 910.34 | 911.44 |
| 9 | 874.36 | 875.5 |
| 10 | 878.35 | 879.48 |
| 11 | 872.34 | 873.37 |
| 12 | 922.34 | 923.38 |
| 13 | 860.34 | 861.36 |
| 14 | 936.35 | 937.43 |
| 15 | 940.4 | 941.43 |
| 16-P1 | 926.39 | 927.45 |
| 16-P2 | 926.39 | 927.45 |
| 17 | 970.42 | 971.42 |
| 18 | 1011.45 | 1012.45 |
| 19 | 998.37 | 999.34 |
| 20 | 1013.46 | 1014.51 |
| 21 | 972.44 | 973.49 |
| 22 | 1004.42 | 1005.41 |
| 23 | 1047.41 | 1048.43 |
| 24 | 984.44 | 985.42 |
| 25 | 938.37 | 939.5 |
| 26 | 968.42 | 969.45 |
| 27 | 1010.43 | 1011.48 |
| 28 | 998.43 | 999.48 |
| 29 | 982.43 | 983.49 |
| 30 | 980.42 | 981.49 |
| 31 | 1025.46 | 1026.52 |
| 32 | 974.44 | 975.51 |
| 33 | 962.44 | 963.5 |
| 34 | 946.45 | 947.5 |
| 35 | 944.43 | 945.49 |
| 36 | 989.48 | 990.53 |
| 37 | 966.46 | 967.49 |
| 38 | 964.44 | 965.51 |
| 39 | 1009.49 | 1010.54 |
| 40 | 930.48 | 931.53 |
| 41 | 928.46 | 929.51 |
| 42 | 973.51 | 974.54 |
| 43 | 1012.44 | 1013.55 |
| 44 | 1026.46 | 1027.61 |
| 45 | 1013.52 | 1014.56 |
| 46 | 952.47 | 953.52 |
| 47 | 982.48 | 983.51 |
| 48 | 900.37 | 901.46 |
| 49 | 984.45 | 985.5 |
| 50 | 1027.48 | 1028.55 |
| 51 | 986.45 | 987.49 |
| 52 | 1029.49 | 1030.56 |
| 53 | 1041.46 | 1042.57 |
| 54 | 896.32 | 897.42 |
| 55 | 898.34 | 897.42 |
| 56-P1 | 983.45 | 984.51 |
| 56-P2 | 983.45 | 984.51 |
| 57-P1 | 1053.49 | 1054.58 |
| 57-P2 | 1053.49 | 1054.58 |
| 58 | 1037.52 | 1038.59 |
| 59-P1 | 1039.54 | 1040.62 |
| 59-P2 | 1039.54 | 1040.62 |
| 60 | 1041.49 | 1042.6 |
| 61 | 930.42 | 931.48 |
| 62 | 944.44 | 945.52 |
| 63 | 932.44 | 933.48 |
| 64 | 946.46 | 947.5 |
| 65 | 968.48 | 969.51 |
| 66 | 970.43 | 971.46 |
| 67 | 996.45 | 997.49 |
| 68 | 994.49 | 995.53 |
| 69 | 932.49 | 933.54 |
| 70 | 934.45 | 935.51 |
| 71 | 972.44 | 973.49 |
| 72 | 970.43 | 971.47 |
| 73 | 996.45 | 997.53 |
| 74 | 1013.46 | 1014.55 |
| 75 | 1001.41 | 1002.47 |
| 76 | 930.47 | 931.53 |
| 77 | 966.46 | 967.51 |
| 78 | 1008.45 | 1009.51 |
| 79 | 1012.44 | 1013.52 |
| 80 | 976.46 | 977.55 |
| 81 | 972.47 | 973.51 |
| 82 | 905.47 | 906.55 |
| 83 | 980.48 | 981.69 |
| 84 | 1036.5 | 1037.7 |
| 85 | 1022.49 | 1023.64 |
| 86 | 992.48 | 993.68 |
| 87 | 1030.42 | 1031.58 |
| 88 | 1006.49 | 1007.67 |
| 89 | 1037.46 | 1038.64 |
| 90 | 1039.48 | 1040.68 |
| 91 | 1053.49 | 1054.71 |
| 92 | 982.49 | 983.67 |
| 93 | 1038.52 | 1039.71 |
| 94 | 1024.5 | 1025.71 |
| 95 | 1032.44 | 1033.62 |
| 96 | 1008.51 | 1009.68 |
| 97 | 1039.48 | 1040.69 |
| 98 | 1041.49 | 1042.71 |
| 99 | 1055.51 | 1056.73 |
| 100 | 1047.47 | 1048.67 |
| 101 | 1039.54 | 1038.7 |
| 102-P1 | 945.4 | 946.3 |
| 102-P2 | 945.4 | 946.3 |
| 103 | 947.41 | 948.31 |
| 104 | 959.41 | 960.33 |
| 105 | 961.43 | 962.36 |
| 106 | 923.43 | 924.36 |
| 107-P1 | 909.42 | 910.34 |
| 107-P2 | 909.42 | 910.34 |
| 108 | 911.43 | 912.34 |
| 109 | 925.45 | 926.37 |
| 110 | 1010.46 | 1011.38 |
| 111 | 1012.48 | 1013.4 |
| 112 | 1024.48 | 1025.39 |
| 113 | 1040.47 | 1041.4 |
| 114 | 1054.49 | 1055.43 |
| 115 | 948.43 | 949.36 |
| 116 | 984.41 | 985.34 |
| 117-P1 | 934.41 | 935.32 |
| 117-P2 | 934.41 | 935.32 |
| 118-P1 | 970.39 | 971.3 |
| 118-P2 | 970.39 | 971.3 |
| 119 | 927.46 | 928.44 |
| 120 | 963.45 | 964.44 |
| 121 | 928.47 | 929.45 |
| 122 | 915.52 | 916.48 |
| 123 | 902.45 | 903.41 |
| 124 | 1053.52 | 1054.52 |
| 125 | 929.43 | 930.4 |
| 126 | 943.44 | 944.45 |
| 127 | 931.44 | 932.43 |
| 128 | 945.46 | 946.46 |
| 129 | 947.41 | 948.39 |
| 130 | 961.43 | 962.44 |
| 131 | 949.43 | 950.41 |
| 132 | 893.45 | 894.43 |
| 133 | 907.46 | 908.45 |
| 134 | 895.46 | 895.45 |
| 135 | 909.48 | 910.48 |
| 136-P1 | 911.43 | 912.39 |
| 136-P2 | 911.43 | 912.39 |
| 137 | 925.45 | 926.44 |
| 138 | 913.45 | 914.41 |
| 139 | 939.46 | 940.43 |
| 140 | 975.45 | 976.44 |
| 141 | 925.45 | 926.42 |
| 142 | 961.43 | 962.4 |
| 143 | 1055.51 | 1056.51 |
| 144 | 1069.52 | 1070.54 |
| 145 | 1057.49 | 1058.49 |
| 146 | 988.52 | 989.52 |
| 147 | 988.52 | 989.52 |
| 148 | 994.49 | 995.49 |
| 149 | 1037.52 | 1038.54 |
| 150 | 1065.55 | 1066.59 |
| 151 | 1081.55 | 1082.58 |
| 152 | 982.39 | 983.32 |
| 153 | 996.41 | 997.36 |
| 154 | 984.41 | 985.34 |
| 155 | 998.43 | 999.39 |
| 156-P1 | 946.41 | 947.34 |
| 156-P2 | 946.41 | 947.34 |
| 157 | 960.43 | 961.36 |
| 158 | 948.43 | 949.36 |
| 159 | 962.44 | 963.37 |
| 160 | 972.41 | 973.35 |
| 161 | 936.43 | 937.36 |
| 162 | 986.43 | 987.36 |
| 163 | 950.44 | 951.38 |
| 164 | 968.48 | 969.41 |
| 165 | 995.51 | 996.47 |
| 166 | 1011.51 | 1012.46 |
| 167 | 970.43 | 971.46 |
| 168 | 982.43 | 983.43 |
| 169 | 1037.41 | 1038.43 |
| 170 | 1022.43 | 1023.45 |
| 171 | 1023.42 | 1024.45 |
| 172 | 1021.44 | 1022.45 |
| 173 | 946.45 | 947.45 |
| 174 | 976.46 | 977.47 |
| 175 | 1008.45 | 1009.47 |
| 176 | 972.47 | 973.48 |
| 177 | 1035.46 | 1036.5 |
| 178 | 1021.44 | 1022.46 |
| 179 | 1021.44 | 1022.47 |
| 180 | 997.43 | 998.45 |
| 181 | 1041.44 | 1042.46 |
| 182 | 1025.46 | 1026.49 |
| 183 | 978.48 | 979.49 |
| 184 | 978.48 | 979.5 |
| 185 | 948.47 | 949.94 |
| 186 | 958.51 | 959.67 |
| 187 | 1026.49 | 1027.66 |
| 188 | 960.47 | 960.82 |
| 189 | 994.43 | 995.56 |
| 190 | 1010.46 | 1010.9 |
| 191 | 1026.46 | 1027.3 |
| 192 | 981.44 | 982.16 |
| 193 | 1067.51 | 1068.73 |
| 194 | 1083.5 | 1084.68 |
| 195 | 952.44 | 953.22 |
| 196 | 981.44 | 982.42 |
| 197 | 1011.45 | 1012.45 |
| 198 | 999.43 | 1000.43 |
| 199 | 1011.45 | 1012.46 |
| 200 | 1020.43 | 1021.43 |
| 201 | 1059.41 | 1060.43 |
| 202 | 1027.42 | 1028.45 |
| 203 | 988.5 | 989.5 |
| 204 | 936.47 | 937.45 |
| 205 | 984.45 | 985.48 |
| 206 | 985.43 | 986.46 |
| 207 | 1015.42 | 1016.46 |
| 208 | 997.43 | 998.46 |
| 209 | 972.44 | 973.42 |
| 210 | 999.45 | 1000.47 |
| 211 | 984.45 | 985.49 |
| 212 | 985.43 | 986.42 |
| 213 | 980.48 | 981.51 |
| 214 | 980.48 | 981.46 |
| 215 | 954.46 | 955.45 |
| 216 | 1009.44 | 1010.45 |
| 217 | 997.49 | 998.5 |
| 218 | 970.43 | 971.45 |
| 219 | 975.45 | 976.46 |
| 220 | 939.46 | 940.48 |
| 221 | 969.48 | 970.49 |
| 222 | 935.41 | 936.46 |
| 223 | 929.44 | 930.49 |
| 224 | 899.43 | 900.48 |
| 225 | 949.43 | 950.47 |
| 226 | 943.46 | 944.49 |
| 227 | 913.45 | 914.48 |
| 228 | 977.46 | 978.51 |
| 229 | 971.49 | 972.52 |
| 230 | 941.48 | 942.52 |
| 231 | 1094.5 | 1095.53 |
| 232 | 1021.44 | 1022.47 |
| 233 | 1026.46 | 1027.45 |
| 234 | 998.46 | 999.5 |
| 235 | 998.46 | 999.51 |
| 236 | 1020.45 | 1021.47 |
| 237 | 1013.46 | 1014.47 |
| 238 | 1048.41 | 1049.41 |
| 239 | 987.47 | 988.48 |
| 240 | 1012.44 | 1013.41 |
| 241 | 1067.53 | 1068.56 |
| 242 | 984.45 | 985.47 |
| 243 | 1045.49 | 1046.53 |
| 244 | 969.47 | 970.49 |
| 245 | 933.49 | 934.52 |
| 246 | 985.44 | 986.46 |
| 247 | 949.46 | 950.49 |
| 248 | 979.47 | 980.49 |
| 249 | 1035.46 | 1036.48 |
| 250 | 1035.46 | 1036.49 |
| 251 | 1034.46 | 1035.3 |
| 252 | 1058.45 | 1059.44 |
| 253 | 1088.44 | 1089.44 |
| 254 | 1074.42 | 1075.44 |
| 255 | 1074.42 | 1075.44 |
| 256 | 1083.51 | 1084.52 |
| 257 | 1076.44 | 1077.46 |
| 258 | 959.41 | 960.41 |
| 259 | 974.45 | 975.46 |
| 260 | 968.48 | 969.48 |
| 261 | 1065.49 | 1066.4 |
| 262 | 1058.42 | 1059.31 |
| 263 | 918.42 | 919.136 |
| 264 | 888.44 | 889.37 |
| 265 | 1038.46 | 1039.37 |
| 266 | 1113.46 | 1114.36 |
| 267 | 1011.45 | 1012.36 |
| 268 | 887.43 | 888.33 |
| 269 | 901.45 | 902.4 |
| 270 | 904.41 | 905.52 |
| 271 | 965.46 | 966.5 |
| 272 | 1036.44 | 1037.31 |
| 273 | 1032.38 | 1033.3 |
| 274 | 915.43 | 916.35 |
| 275 | 921.4 | 922.31 |
| 276 | 967.42 | 968.34 |
| 277 | 1041.49 | 1042.43 |
| 278 | 1030.42 | 1031.28 |
| 279 | 966.46 | 967.32 |
| 280 | 951.45 | 952.33 |
| 281 | 1044.44 | 1045.27 |
| 282 | 1052.48 | 1053.32 |
| 283 | 1068.45 | 1069.28 |
| 284 | 1049.43 | 1050.25 |
| 285 | 968.42 | 969.28 |
| 286 | 1011.45 | 1012.29 |
| 287 | 994.43 | 995.29 |
| 288 | 1027.48 | 1028.2 |
| 289 | 1041.49 | 1042.2 |
| 290 | 952.44 | 953.32 |
| 291 | 968.42 | 969.27 |
| 292 | 982.43 | 983.32 |
| 293 | 980.48 | 981.35 |
| 294 | 966.46 | 967.38 |
| 295 | 982.43 | 983.31 |
| 296 | 996.45 | 997.31 |
| 297 | 1021.44 | 1022.28 |
| 298 | 1046.39 | 1047.25 |
| 299 | 1042.44 | 1043.25 |
| 300 | 1060.41 | 1061.21 |
| 301 | 1000.43 | 1001.2 |
| 302 | 1014.46 | 1015.12 |
| 303 | 1026.47 | 1027.23 |
| 304 | 984.46 | 985.22 |
| 305 | 1021.44 | 1022.17 |
| 306 | 1031.44 | 1032.23 |
| 307 | 1047.41 | 1048.15 |
| 308 | 1013.46 | 1014.2 |
| 309 | 1049.43 | 1050.16 |
| 310 | 1005.47 | 1006.21 |
| 311 | 946.42 | 947.27 |
| 312 | 1067.51 | 1068.31 |
| 313 | 1067.51 | 1068.3 |
| 314 | 960.44 | 961.29 |
| 315 | 1051.54 | 1052.35 |
| 316 | 1051.54 | 1052.33 |
| 317 | 1065.55 | 1066.34 |
| 318 | 1065.55 | 1066.37 |
| 319 | 989.46 | 990.28 |
| 320 | 990.46 | 991.27 |
| 321 | 962.41 | 963.25 |
| 322 | 919.42 | 920.26 |
| 323 | 1001.44 | 1002.29 |
| 324 | 1018.45 | 1019.27 |
| 325 | 1067.53 | 1068.37 |
| 326 | 1067.53 | 1068.36 |
| 327 | 1083.5 | 1084.35 |
| 328 | 1081.55 | 1082.39 |
| 329 | 1081.55 | 1082.38 |
| 330 | 948.4 | 949.22 |
| 331 | 905.4 | 906.26 |
| 332 | 995.47 | 996.3 |
| 333 | 1051.48 | 1052.32 |
| 334 | 1035.51 | 1036.36 |
| 335 | 1017.51 | 1018.20 |
| 336 | 1078.52 | 1079.30 |
| 337 | 1049.52 | 1050.14 |
| 338 | 1060.48 | 1062.12 |
| 339 | 1109.52 | 1110.17 |
| 340 | 1080.49 | 1081.14 |
| 341 | 1064.51 | 1065.18 |
| 342 | 1051.48 | 1052.17 |
| 343 | 1002.44 | 1003.14 |
| 344 | 1079.51 | 1080.13 |
| 345 | 1099.45 | 1100.08 |
| 346 | 1120.54 | 1121.16 |
| 347 | 1106.56 | 1107.18 |
| 348 | 1083.48 | 1084.13 |
| 349 | 1084.48 | 1085.14 |
| 350 | 1047.5 | 1048.12 |
| 351 | 1075.51 | 1076.21 |
| 352 | 1128.52 | 1129.12 |
| 353 | 1062.55 | 1063.26 |
| 354 | 1129.45 | 1130.17 |
| 355 | 1077.49 | 1079.03 |
| 356 | 1166.58 | 1167.33 |
| 357 | 1090.52 | 1091.26 |
| 358 | 1077.53 | 1078.23 |
| 359 | 1079.51 | 1080.26 |
| 360 | 1092.54 | 1093.70 |
| 361 | 1095.5 | 1096.21 |
| 362 | 1104.54 | 1105.27 |
| 363 | 1091.51 | 1093.21 |
| 364 | 1120.54 | 1121.90 |
| 365 | 1011.52 | 1012.93 |
| 366 | 1093.52 | 1095.00 |
| 367 | 1095.5 | 1096.98 |
| 368 | 1155.55 | 1157.08 |
| 369 | 1077.49 | 1078.99 |
| 370 | 1095.52 | 1097.03 |
| 371 | 1093.52 | 1095.01 |
| 372 | 1158.56 | 1160.05 |
| 373 | 1109.52 | 1110.93 |
| 374 | 1092.54 | 1093.94 |
| 375 | 1057.47 | 1059.09 |
| 376 | 1082.56 | 1084.03 |
| 377 | 1021.49 | 1022.82 |
| 378 | 1102.49 | 1103.88 |
| 379 | 1090.49 | 1092.09 |
| 380 | 1143.55 | 1145.14 |
| 381 | 1118.56 | 1120.01 |
| 382 | 1066.49 | 1068.17 |
| 383 | 1133.57 | 1135.25 |
| 384 | 1106.56 | 1108.21 |
| 385 | 1073.46 | 1074.99 |
| 386 | 1101.47 | 1103.21 |
| 387 | 1092.54 | 1094.09 |
| 388 | 1092.54 | 1094.08 |
| 389 | 1093.52 | 1095.13 |
| 390 | 1106.52 | 1107.98 |
| 391 | 1063.55 | 1064.89 |
| 392 | 957.44 | 958.48 |
| 393 | 957.44 | 958.38 |
| 394 | 944.4 | 945.46 |
| 395 | 1063.48 | 1064.97 |
| 396 | 1094.52 | 1096 |
| 397 | 1076.51 | 1077.72 |
| 398 | 981.47 | 982.73 |
| 399 | 997.51 | 998.91 |
| 400 | 997.51 | 998.92 |
| 401 | 1080.54 | 1081.93 |
| 402 | 1068.48 | 1069.98 |
| 403 | 1093.52 | 1094.98 |
| 404 | 1079.51 | 1080.7 |
| 405 | 1092.54 | 1093.73 |
| 406 | 1049.5 | 1050.95 |
| 407 | 1049.5 | 1050.95 |
| 408 | 1079.47 | 1080.68 |
| 409 | 1092.54 | 1093.52 |
| 410 | 1122.55 | 1123.67 |
| 411 | 1077.53 | 1078.7 |
| 412 | 1077.53 | 1078.67 |
| 413 | 1124.55 | 1125.74 |
| 414 | 1079.51 | 1080.56 |
| 415 | 1111.5 | 1112.66 |
| 416 | 1155.51 | 1156.35 |
| 417 | 1077.53 | 1078.7 |
| 418 | 1097.48 | 1098.73 |
| 419 | 1124.53 | 1125.69 |
| 420 | 1179.57 | 1180.49 |
| 421 | 1083.5 | 1084.68 |
| 422 | 1081.54 | 1082.82 |
| 423 | 939.45 | 940.41 |
| 424 | 958.43 | 959.47 |
| 425 | 954.42 | 955.42 |
| 426 | 910.37 | 911.44 |

Table 4, below, provides the full chemical structure for each exemplified compound in Table 2.

TABLE 4
Chemical Structure for Exemplary
Compounds Described in Table 2

| Ex. | |
|---|---|
| No. | Chemical Structure |
| 1 | 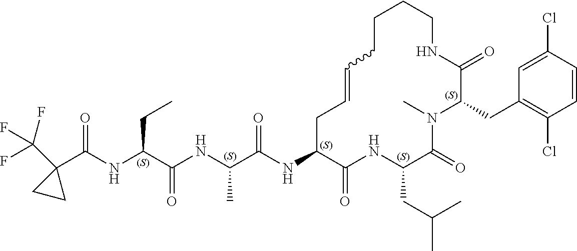 |
| 2 | 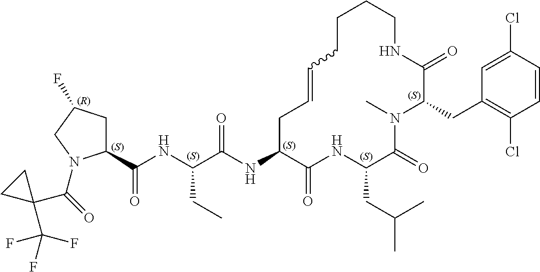 |
| 3 | 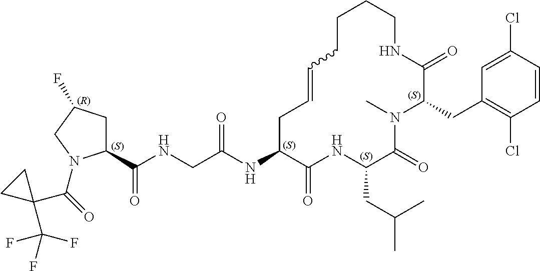 |
| 4 | 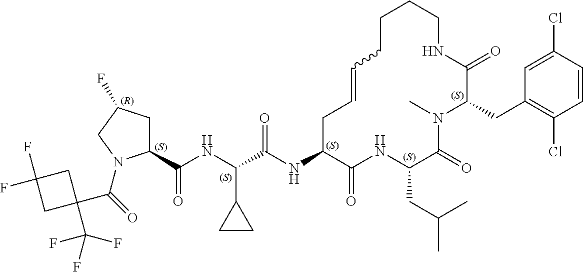 |
| 5- P1/ 5- P2 | 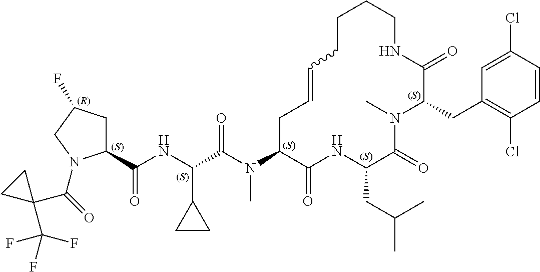 |
| 6- P1/ 6- P2 | 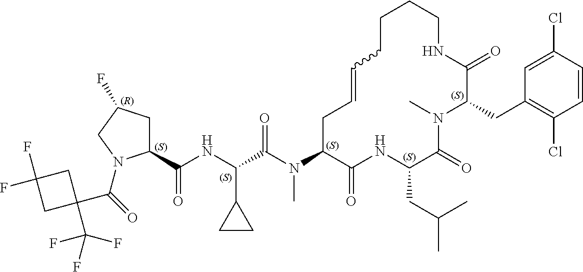 |
| 7 | 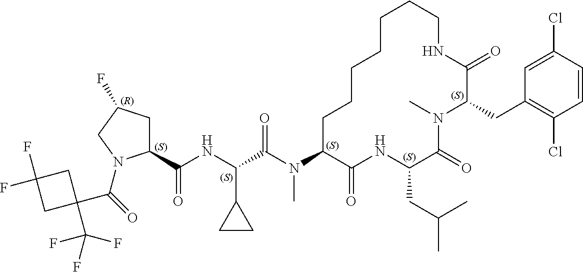 |
| 8 | 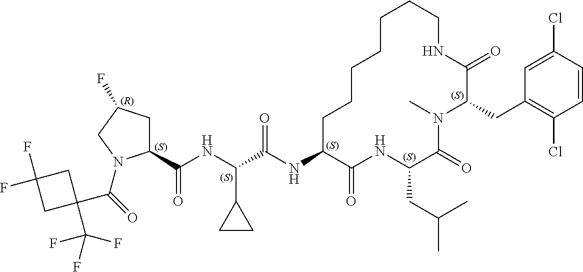 |
| 9 | 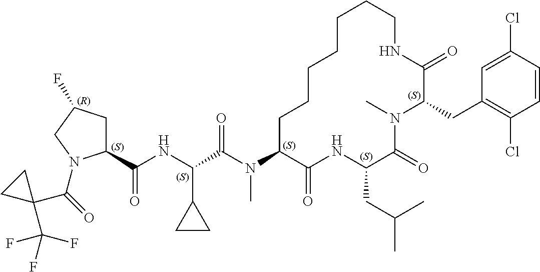 |
| 10 | 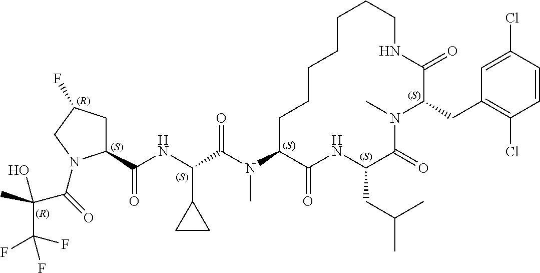 |
| 11 | 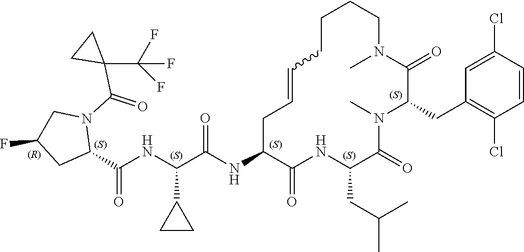 |
| 12 | 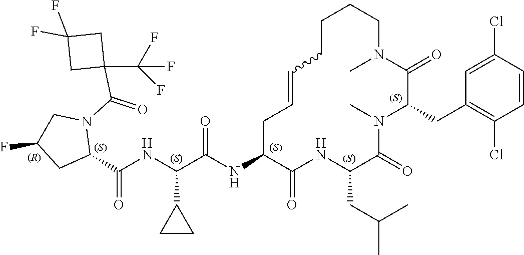 |
| 13 | 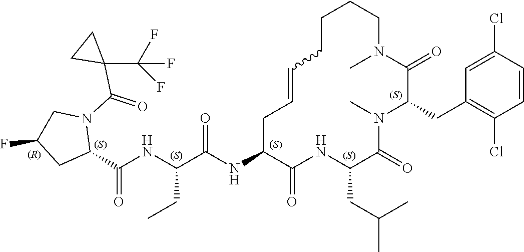 |
| 14 | 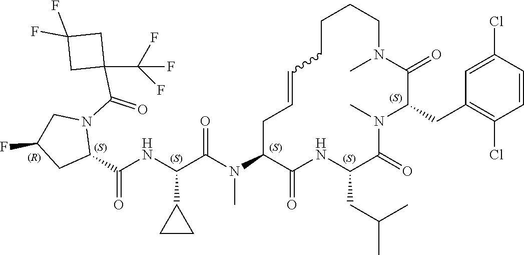 |
| 15 | 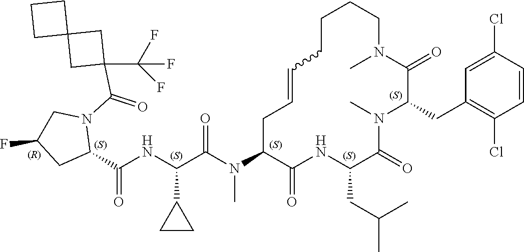 |
| 16- P1/ 16- P2 | 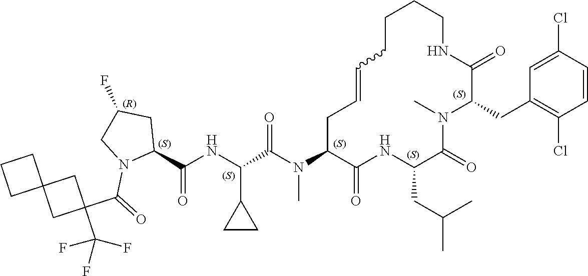 |
| 17 | 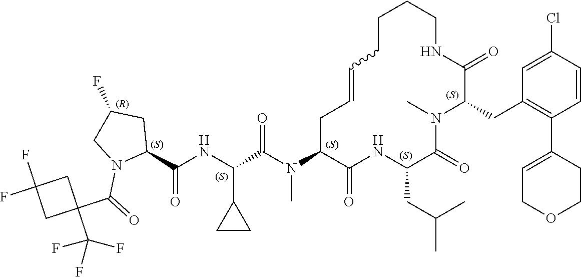 |
| 18 | 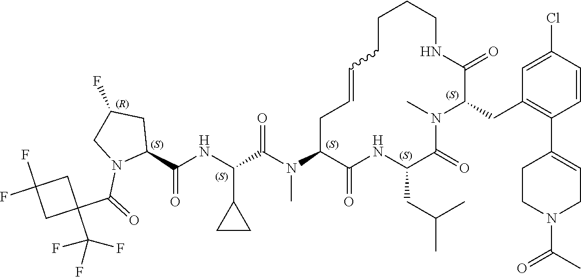 |
| 19 | 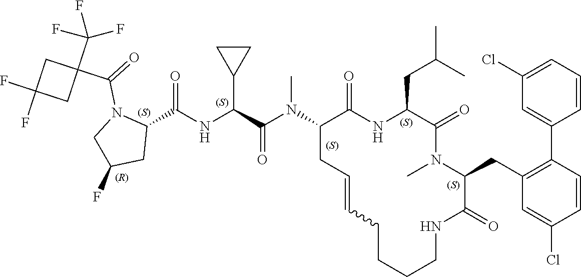 |
| 20 | 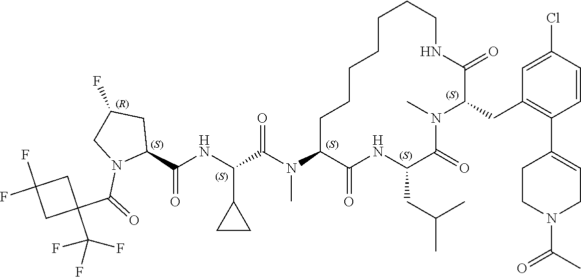 |
| 21 | 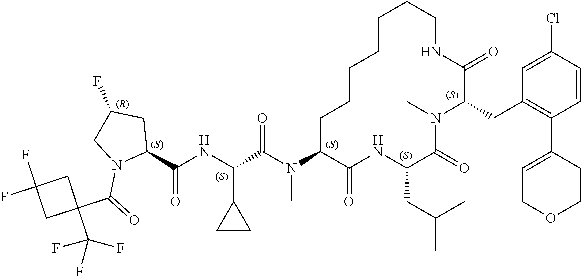 |
| 22 | 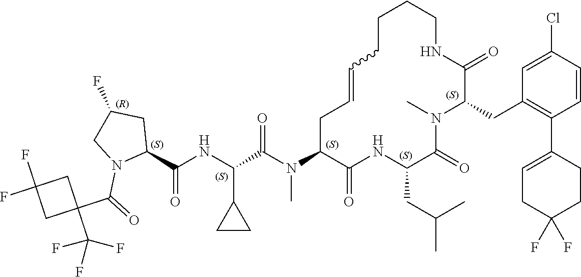 |
| 23 | 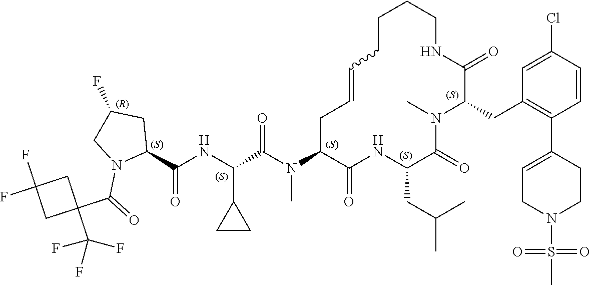 |
| 24 | 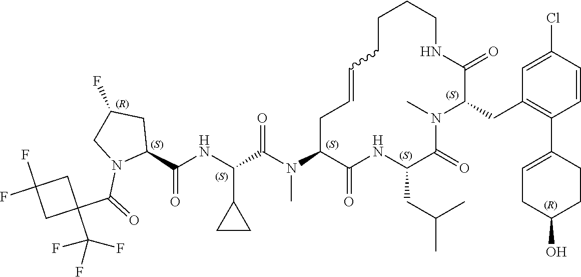 |
| 25 | 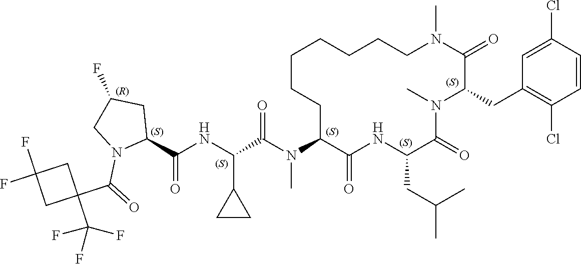 |
| 26 | 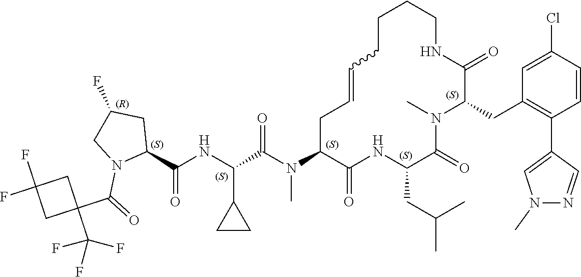 |
| 27 | 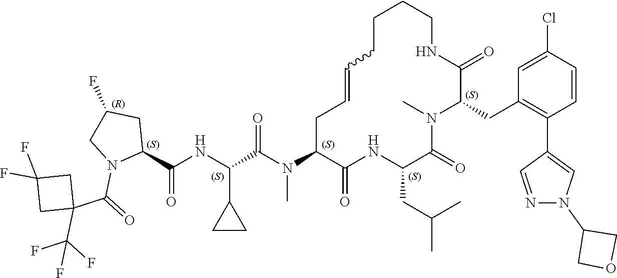 |
| 28 | 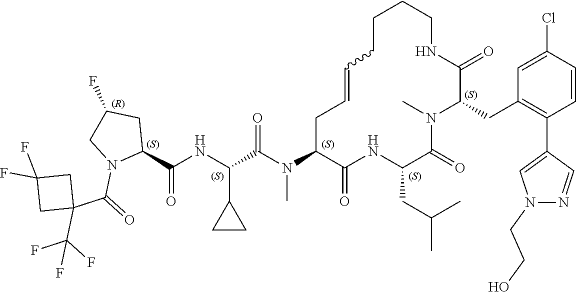 |
| 29 | 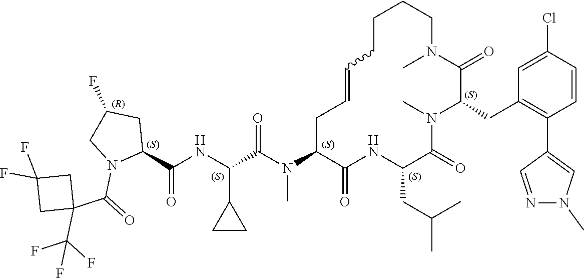 |
| 30 | 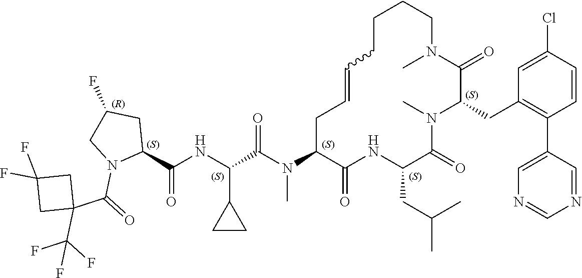 |
| 31 | 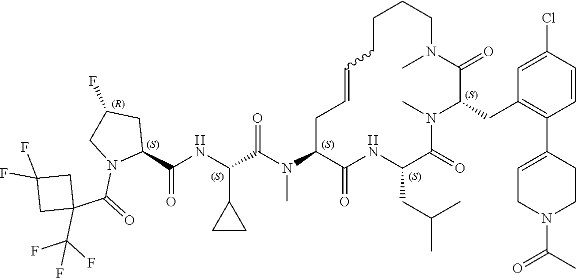 |
| 32 | 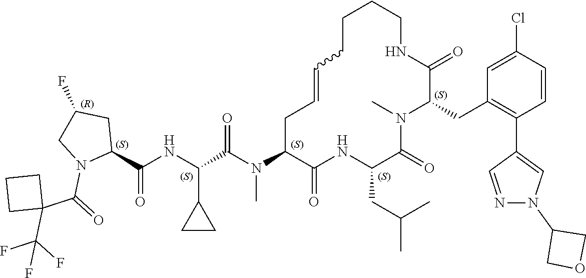 |
| 33 | 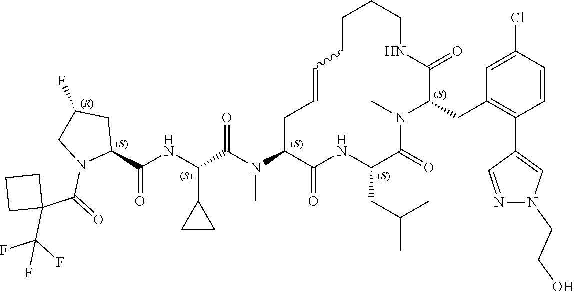 |
| 34 | 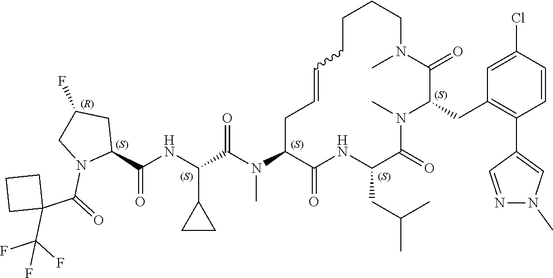 |
| 35 | 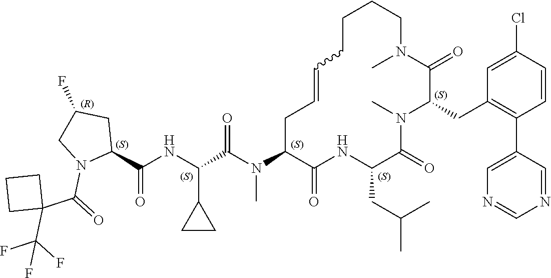 |
| 36 | 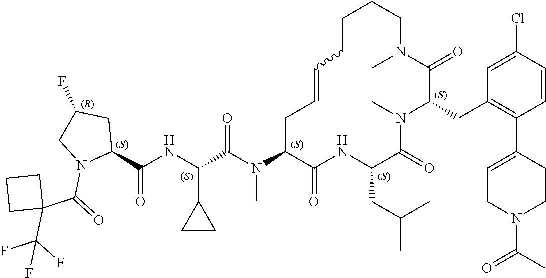 |
| 37 | 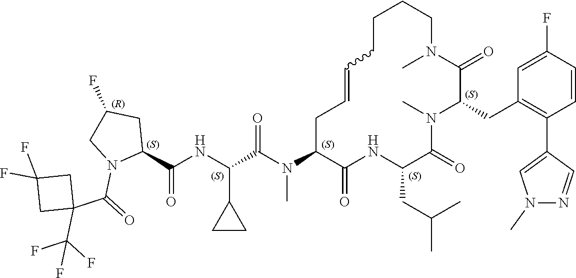 |
| 38 | 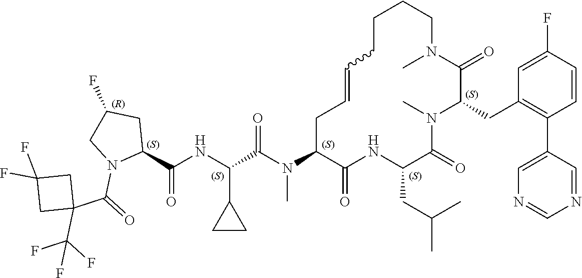 |
| 39 | 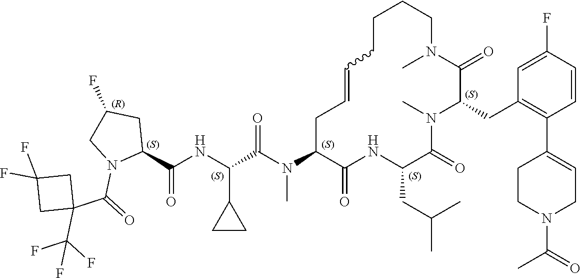 |
| 40 | 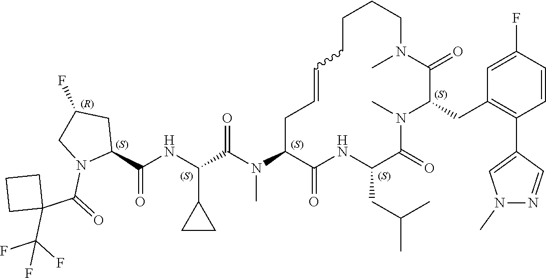 |
| 41 | 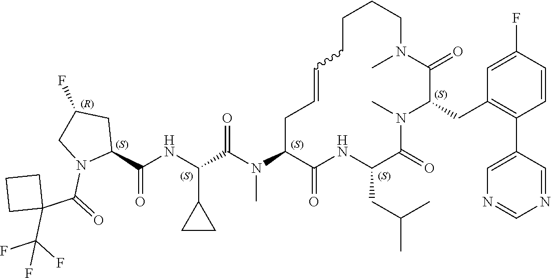 |
| 42 | 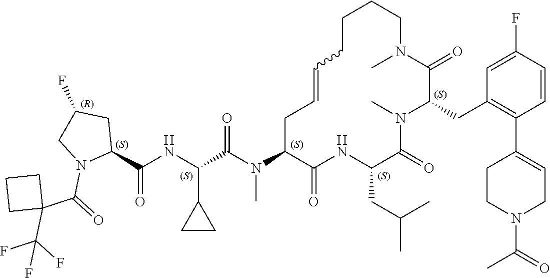 |
| 43 | 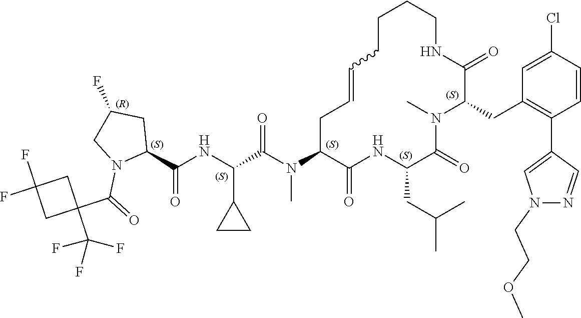 |
| 44 | 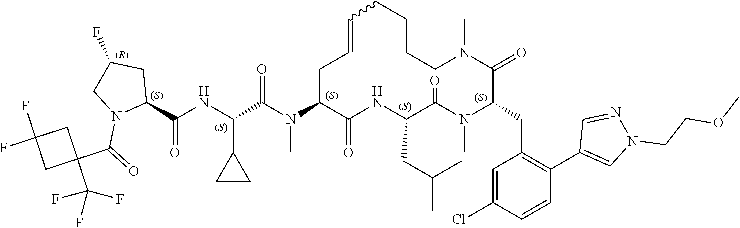 |
| 45 | 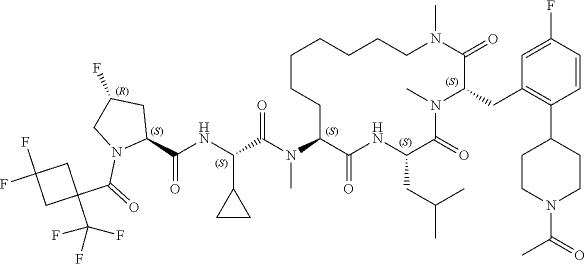 |
| 46 | 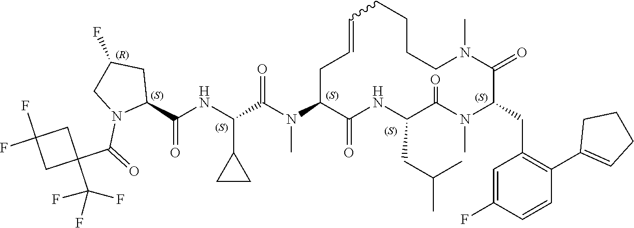 |
| 47 | 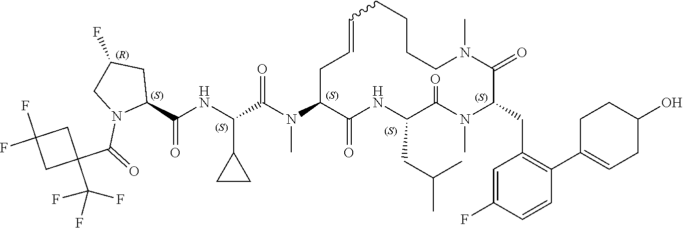 |
| 48 | 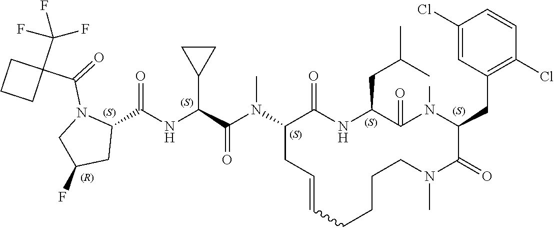 |
| 49 | 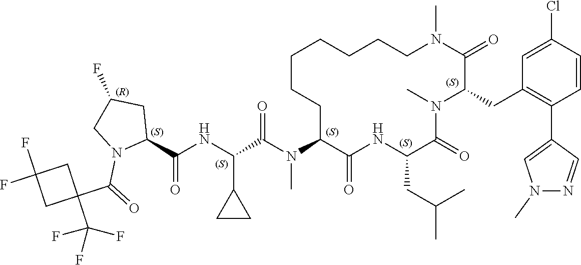 |
| 50 | 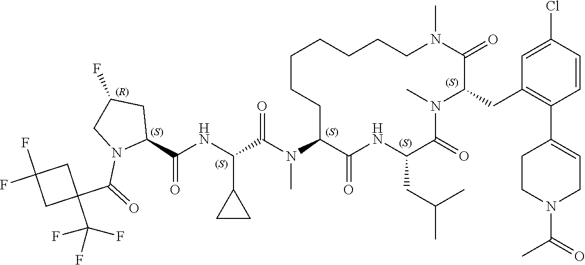 |
| 51 | 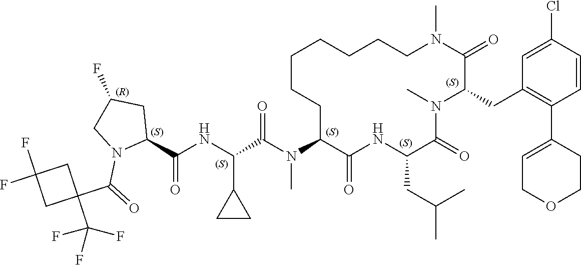 |
| 52 | 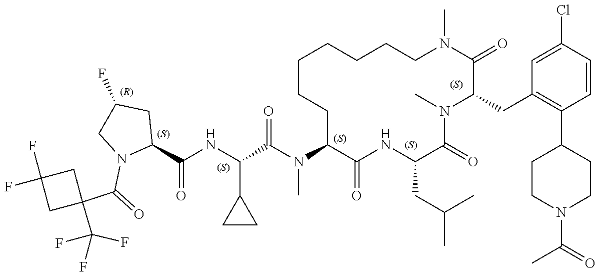 |
| 53 | 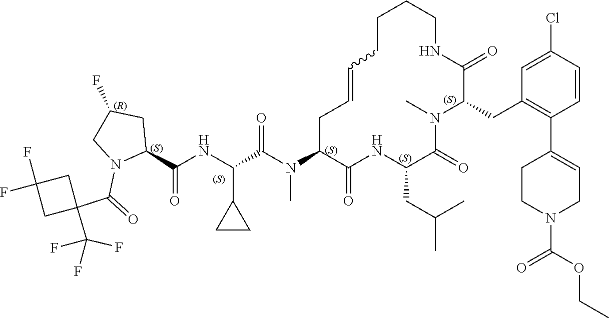 |
| 54 | 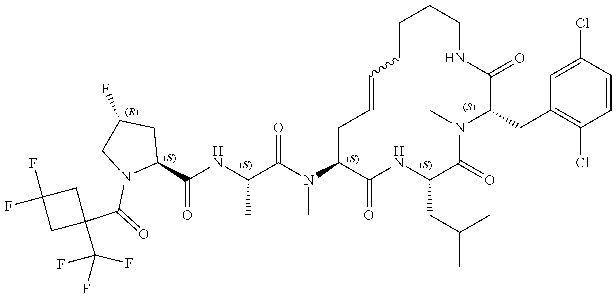 |
| 55 | 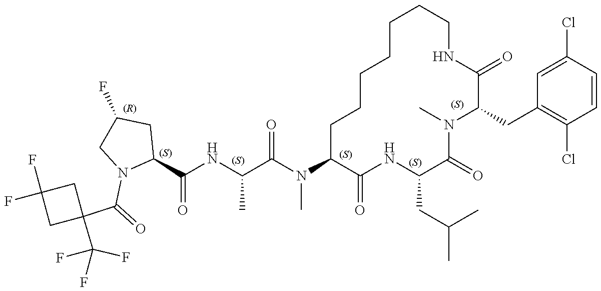 |
| 56- P1/ 56- P2 | 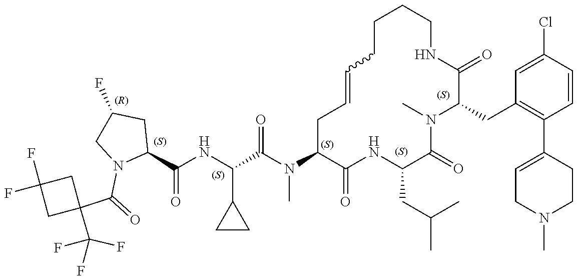 |
| 57- P1/ 57- P2 | 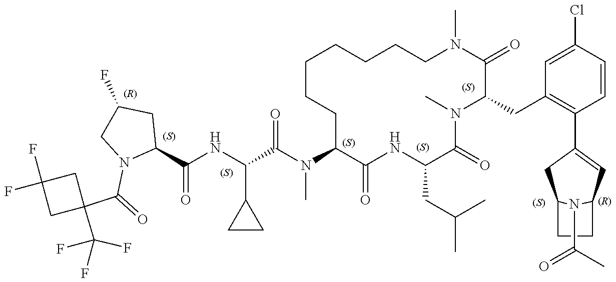 |
| 58 | 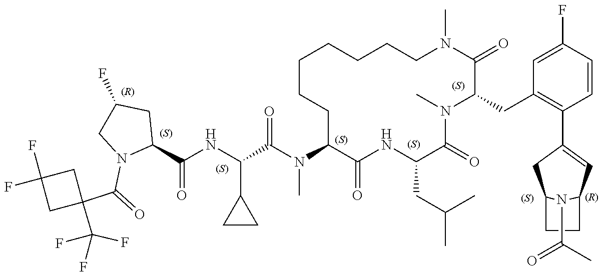 |
| 59- P1/ 59- P2 | 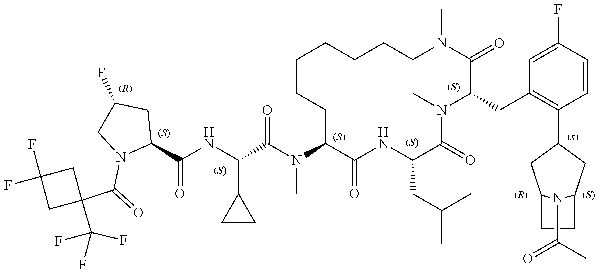 |
| 60 | 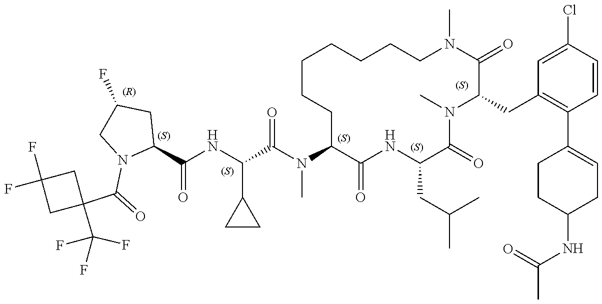 |
| 61 | 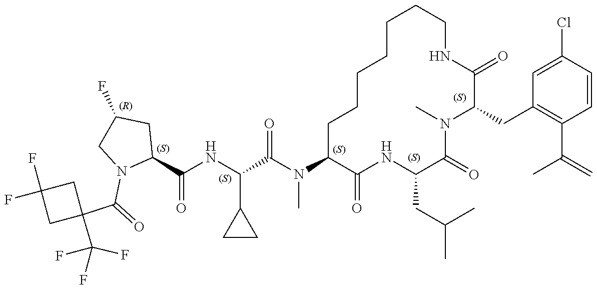 |
| 62 | 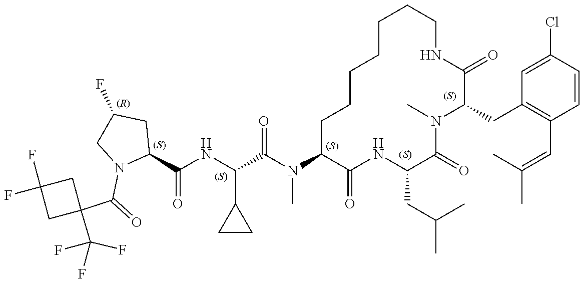 |
| 63 | 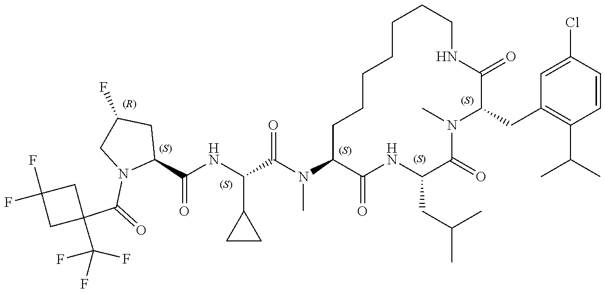 |
| 64 | 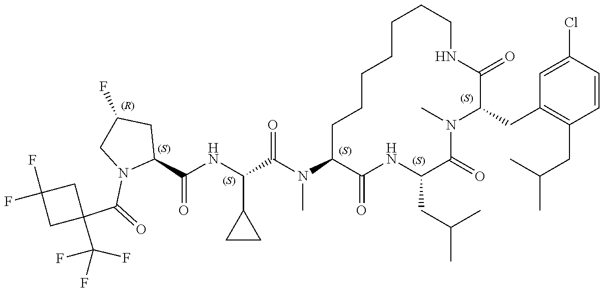 |
| 65 | 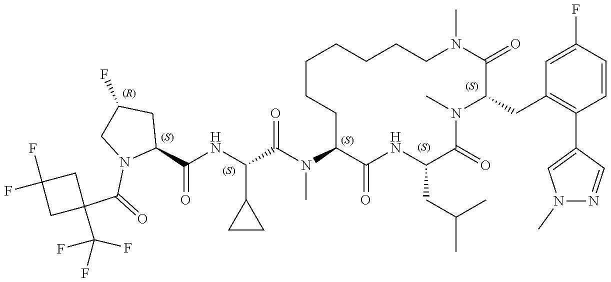 |
| 66 | 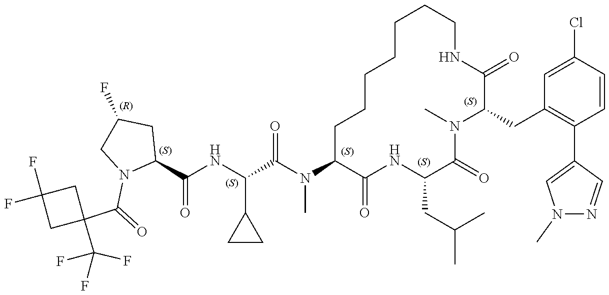 |
| 67 | 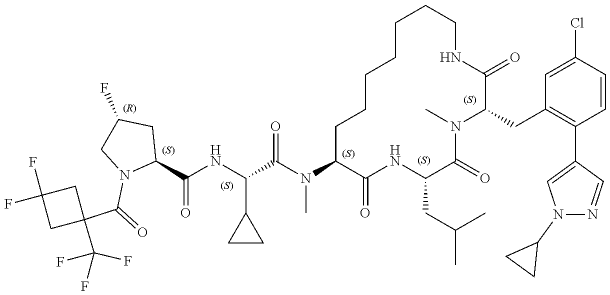 |
| 68 | 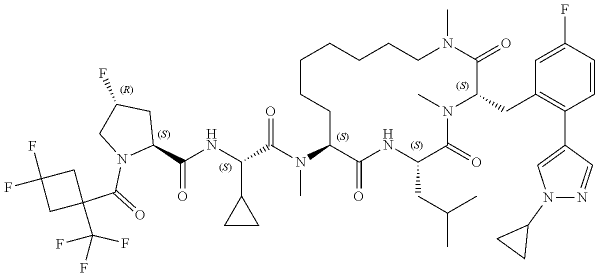 |
| 69 | 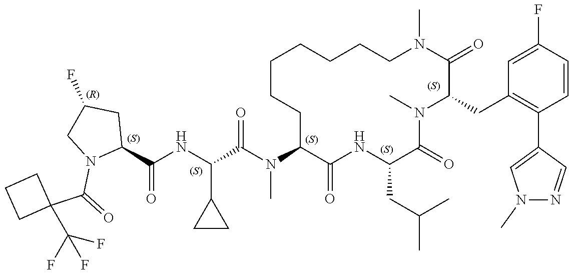 |
| 70 | 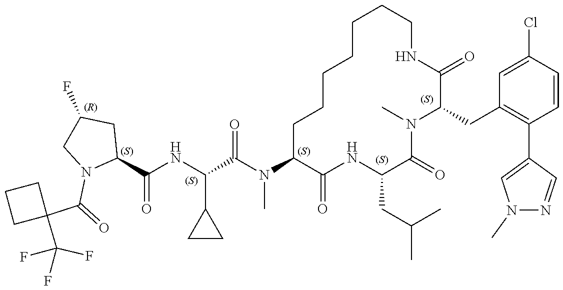 |
| 71 | 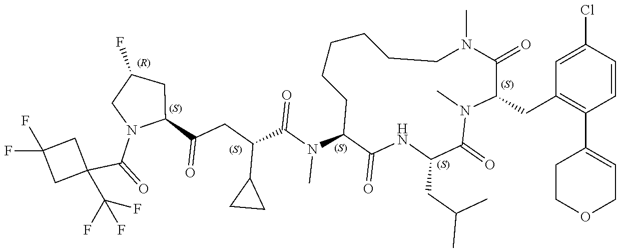 |
| 72 | 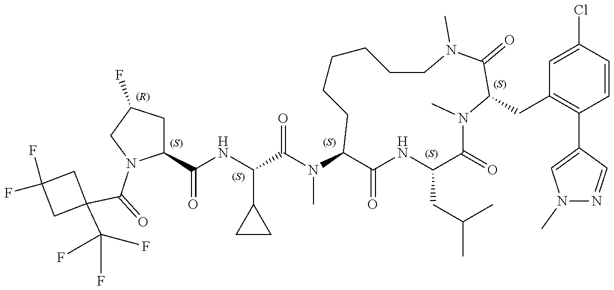 |
| 73 | 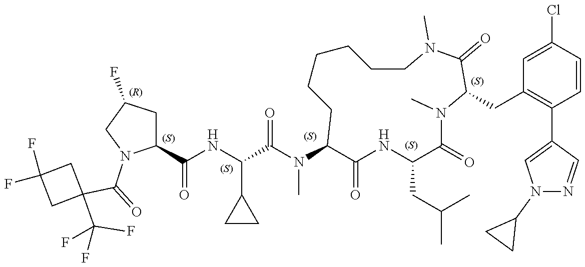 |
| 74 | 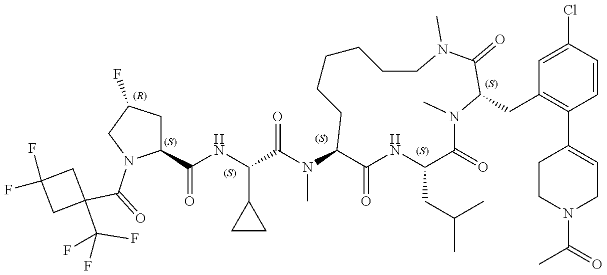 |
| 75 | 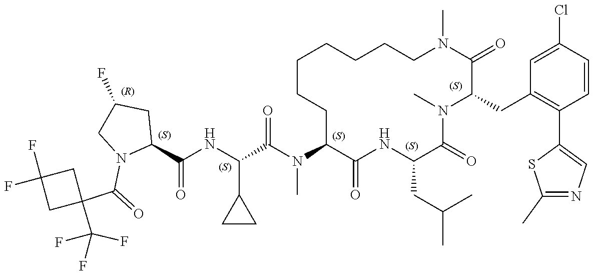 |
| 76 | 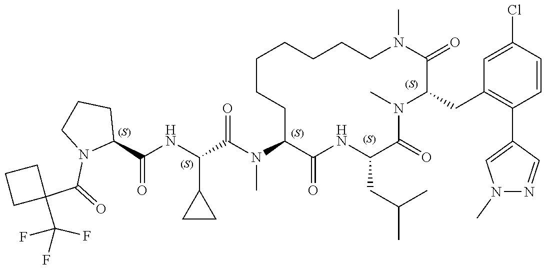 |
| 77 | 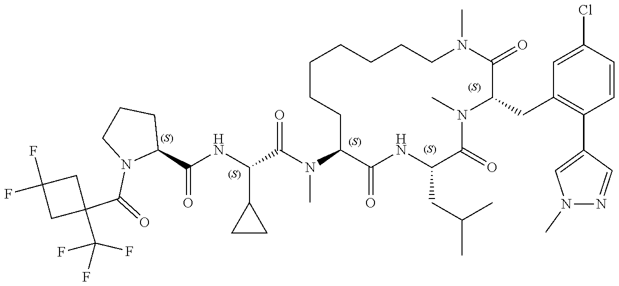 |
| 78 | 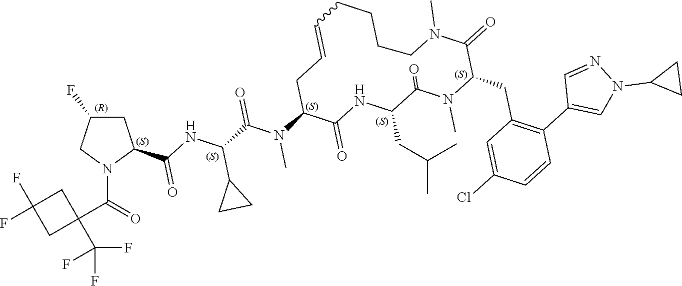 |
| 79 | 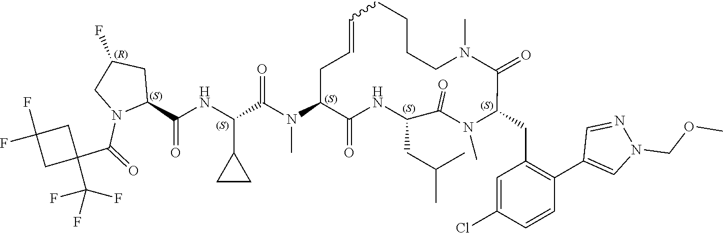 |
| 80 | 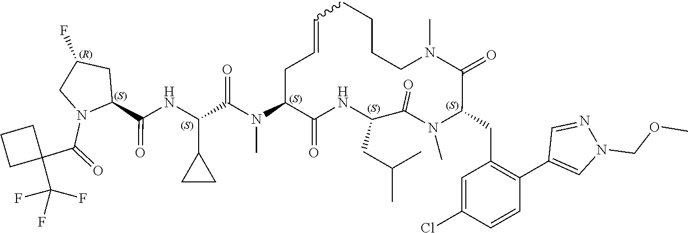 |
| 81 | 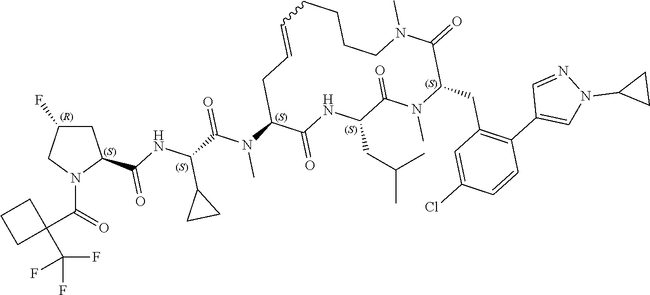 |
| 82 | 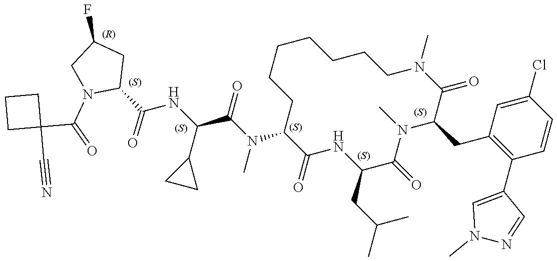 |
| 83 | 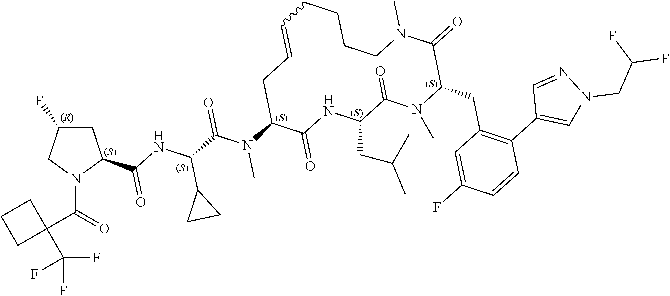 |
| 84 | 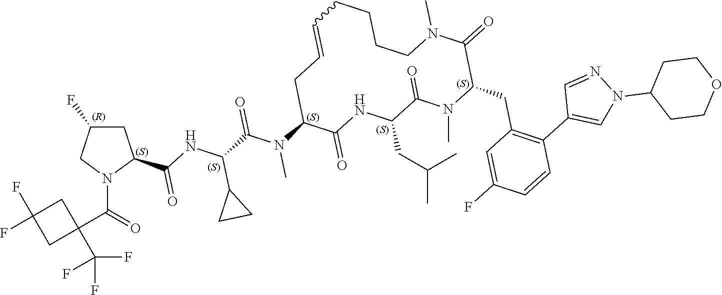 |
| 85 | 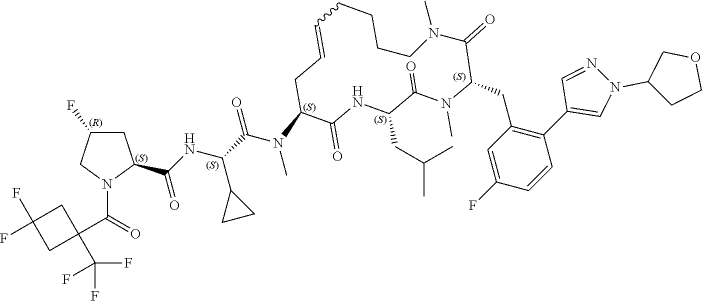 |
| 86 | 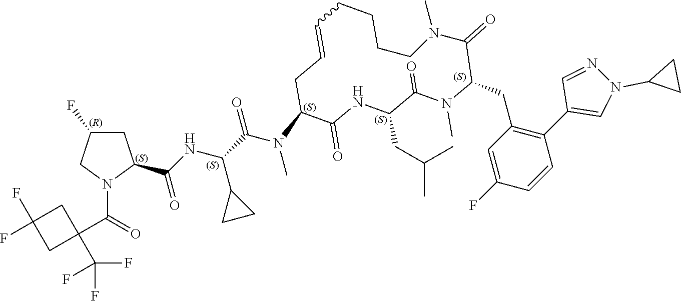 |
| 87 | 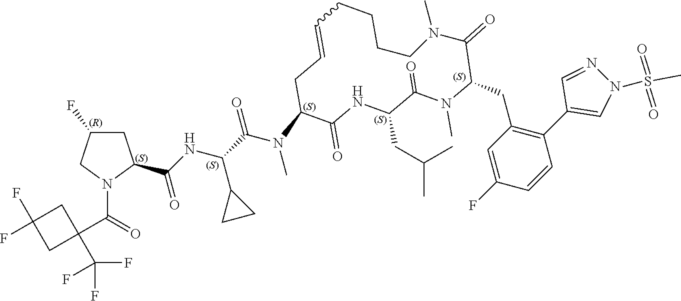 |
| 88 | 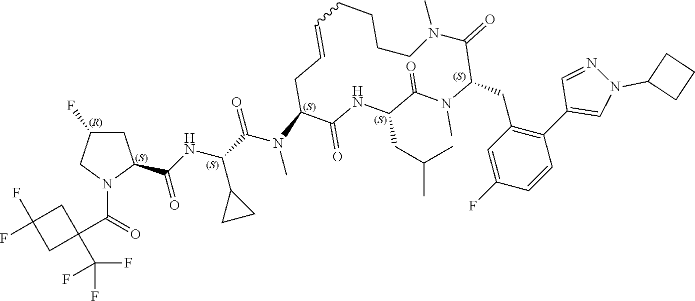 |
| 89 | 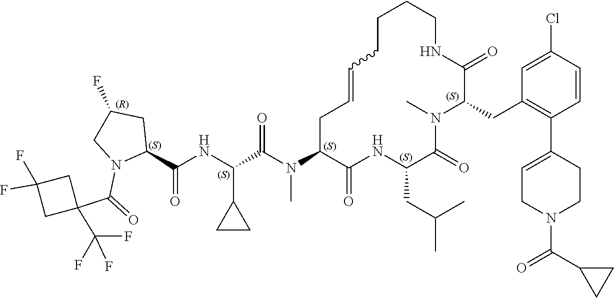 |
| 90 | 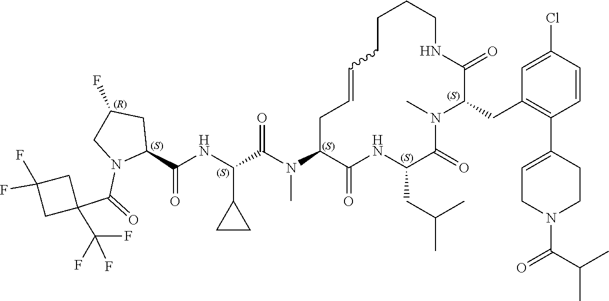 |
| 91 | 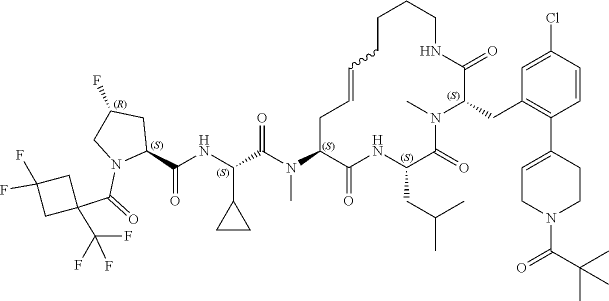 |
| 92 | 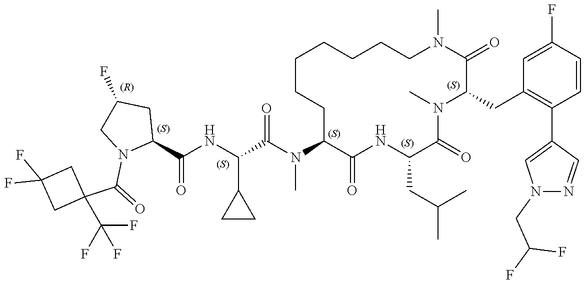 |
| 93 | 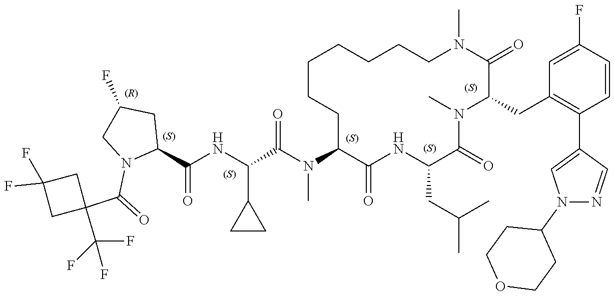 |
| 94 | 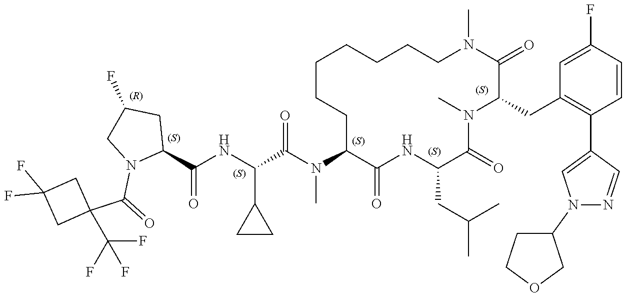 |
| 95 | 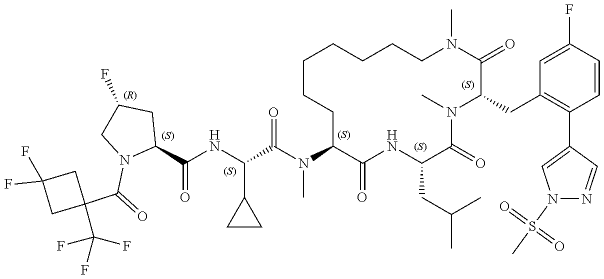 |
| 96 | 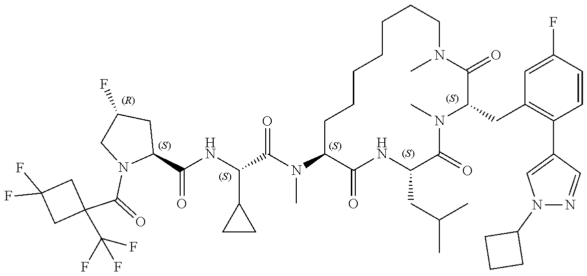 |
| 97 | 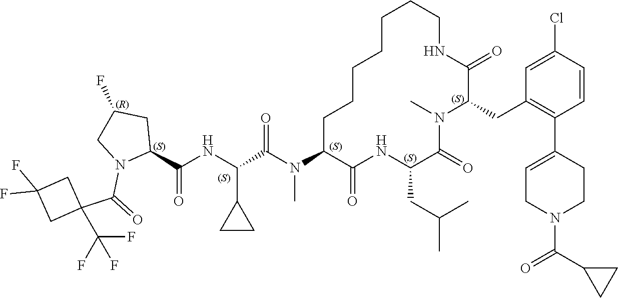 |
| 98 | 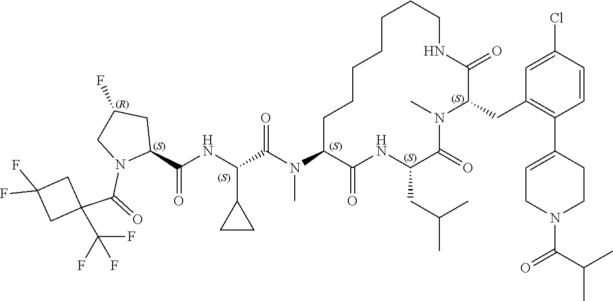 |
| 99 | 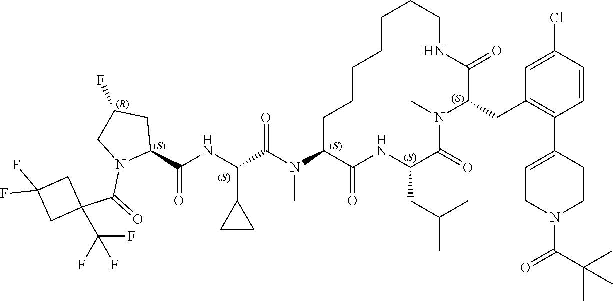 |
| 100 | 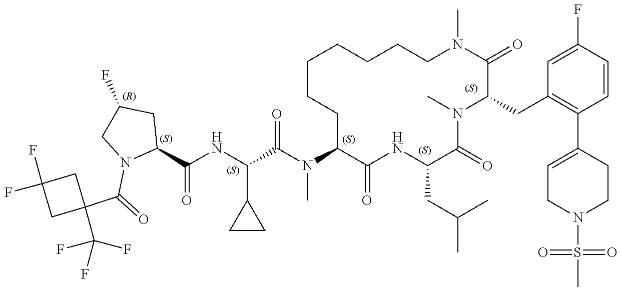 |
| 101 | 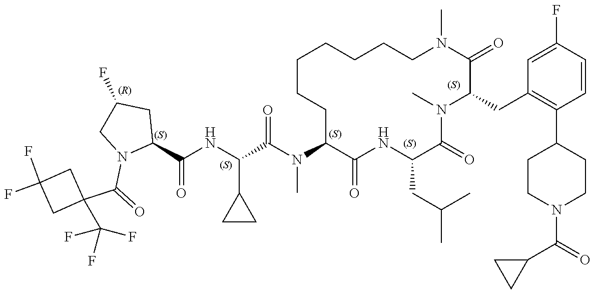 |
| 102- P1/ 102- P2 | 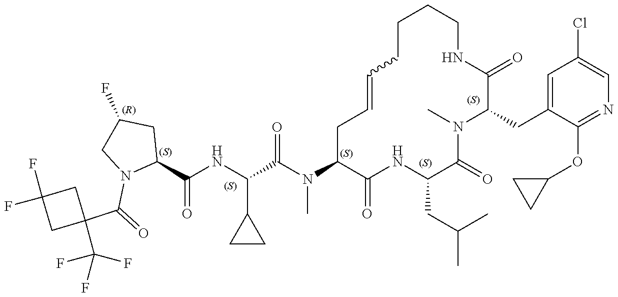 |
| 103 | 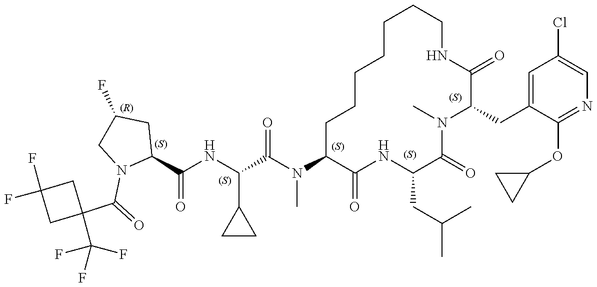 |
| 104 | 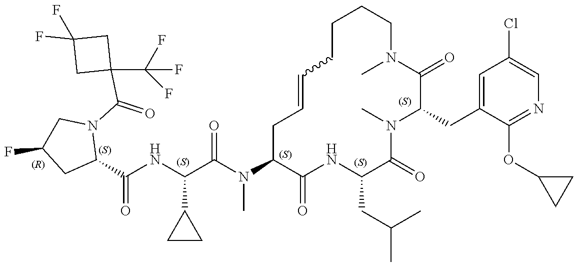 |
| 105 | 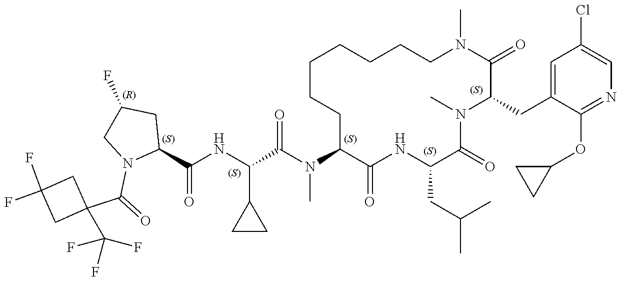 |
| 106 | 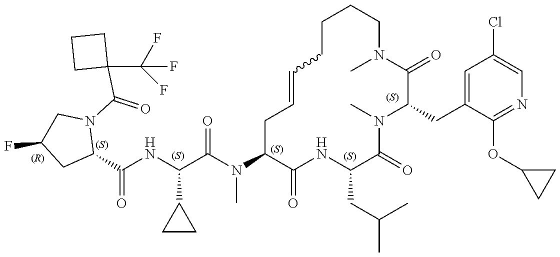 |
| 107- P1/ 107- P2 | 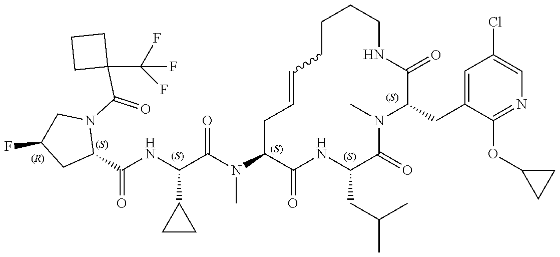 |
| 108 | 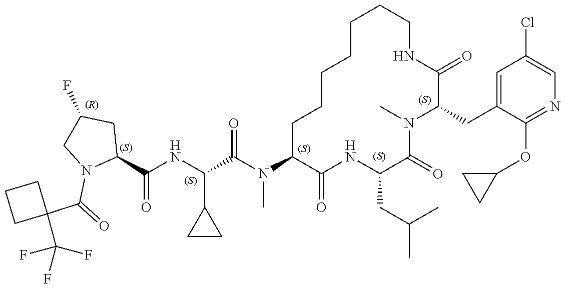 |
| 109 | 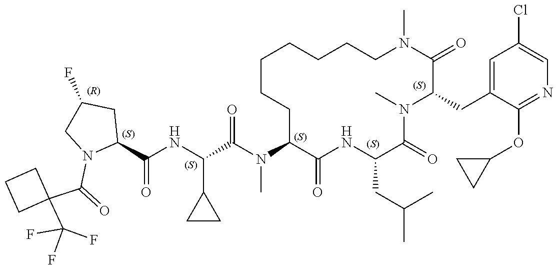 |
| 110 | 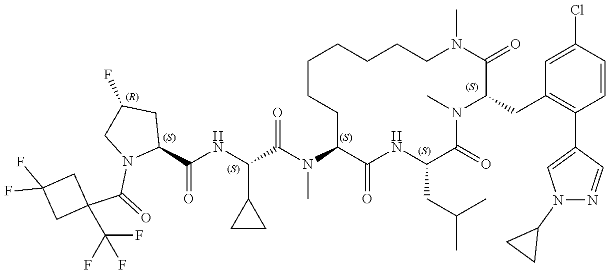 |
| 111 | 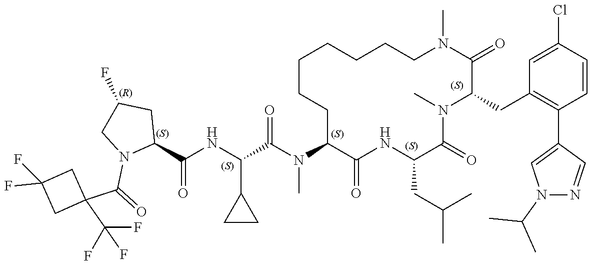 |
| 112 | 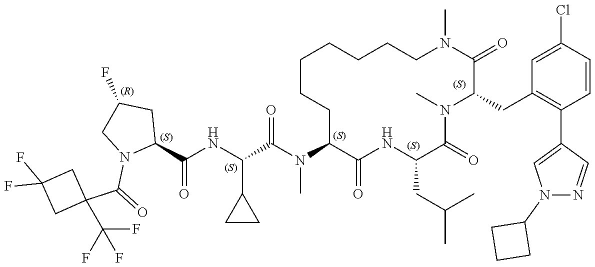 |
| 113 | 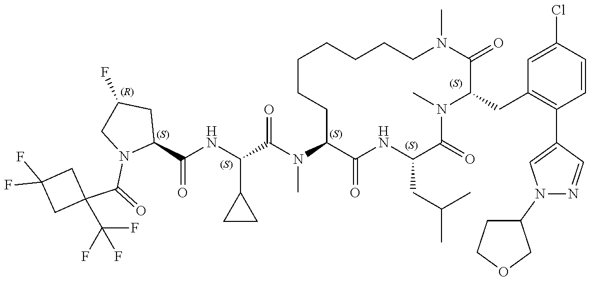 |
| 114 | 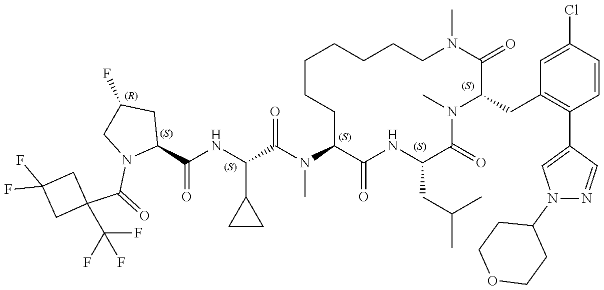 |
| 115 | 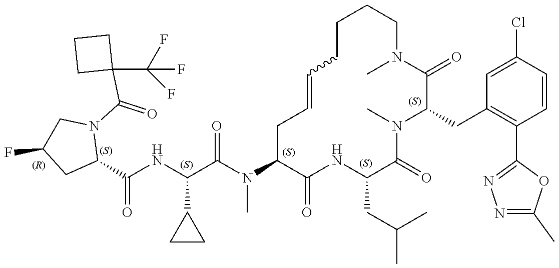 |
| 116 | 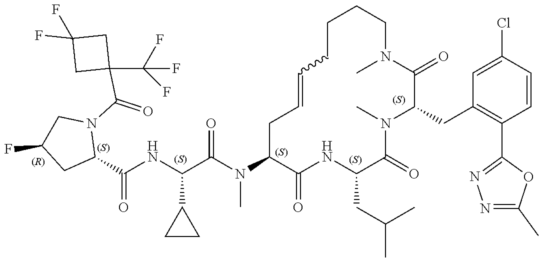 |
| 117- P1/ 117- P2 | 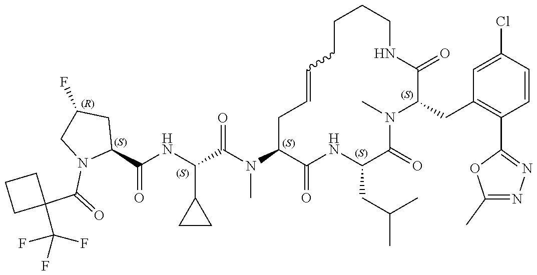 |
| 118- P1/ 118- P2 | 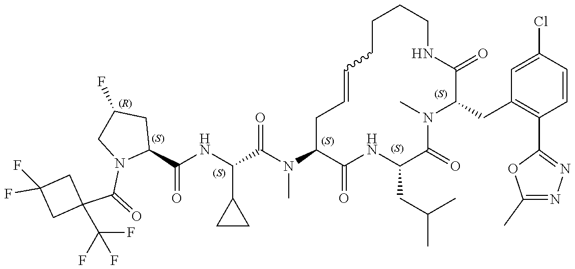 |
| 119 | 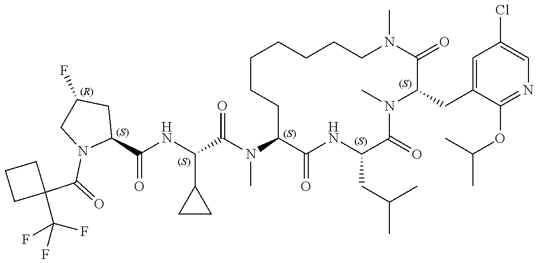 |
| 120 | 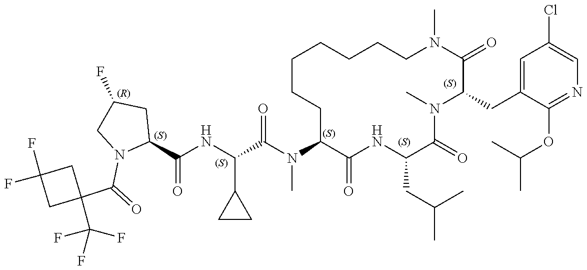 |
| 121 | 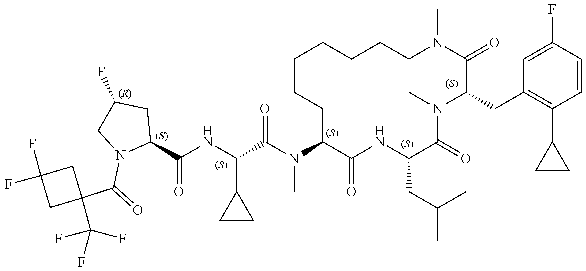 |
| 122 | 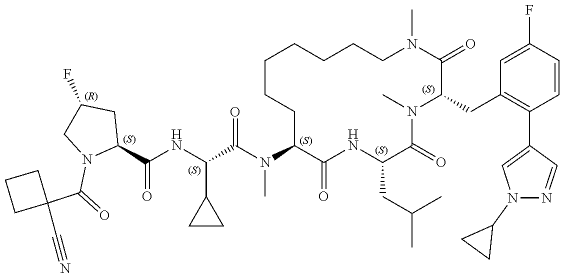 |
| 123 | 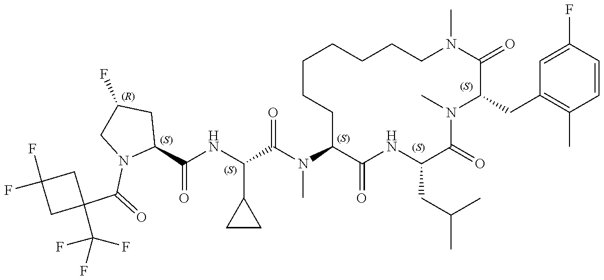 |
| 124 | 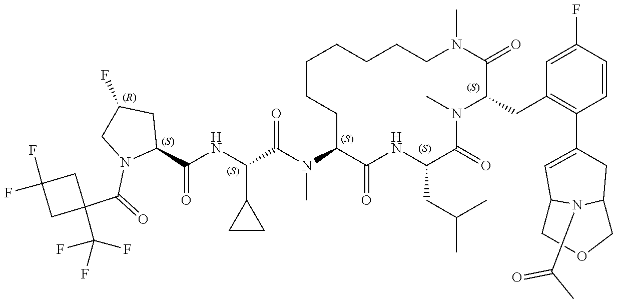 |
| 125 | 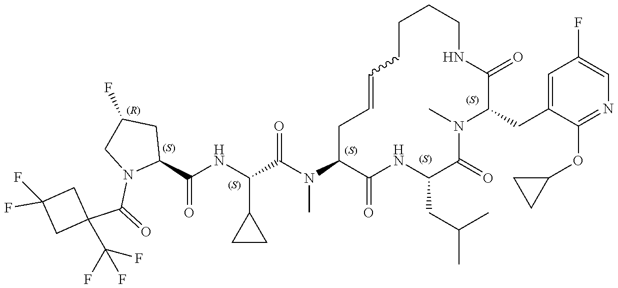 |
| 126 | 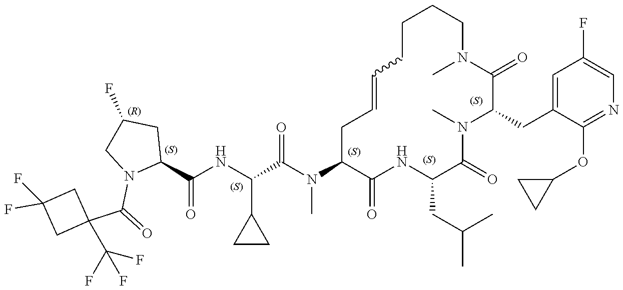 |
| 127 | 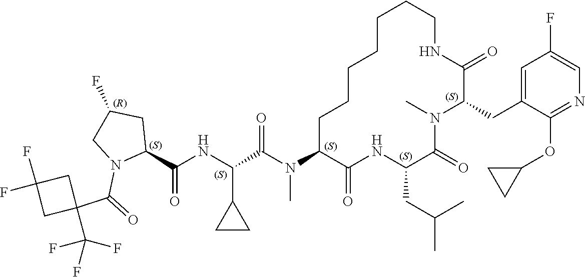 |
| 128 | 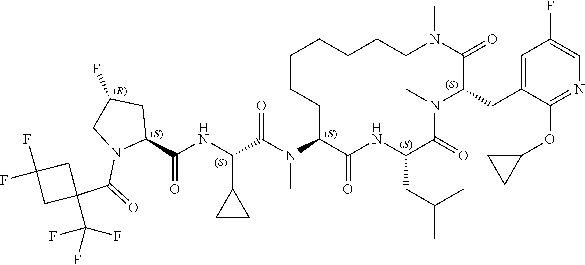 |
| 129 | 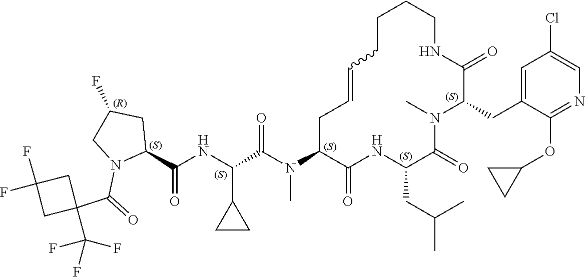 |
| 130 | 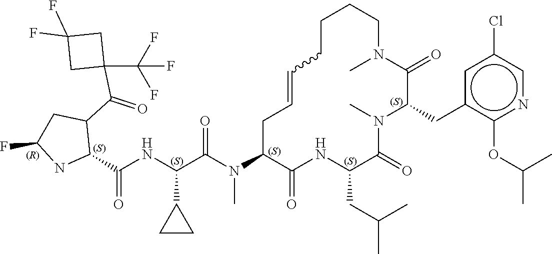 |
| 131 | 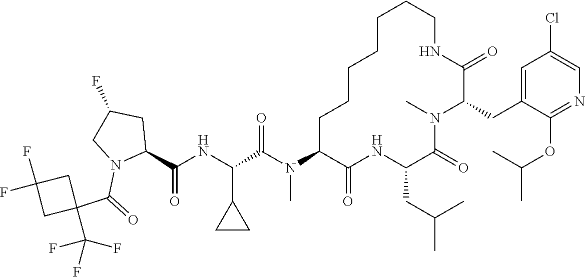 [SMILES: CC(C)C[C@@H]1NC(=O)[C@@H](N(C)C(=O)[C@@H](NC(=O)[C@@H]2C[C@@H](F)CN2C(=O)C2(C(F)(F)F)CC(F)(F)C2)C2CC2)CCCCCCCNC(=O)[C@H](Cc2cc(Cl)cnc2OC(C)C)N(C)C1=O] |
| 132 | 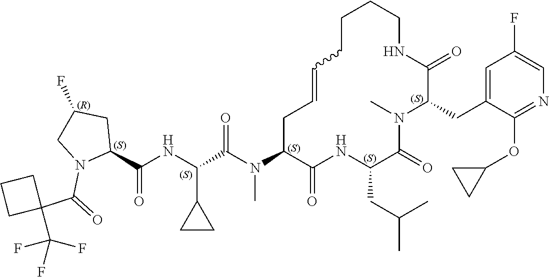 |
| 133 | 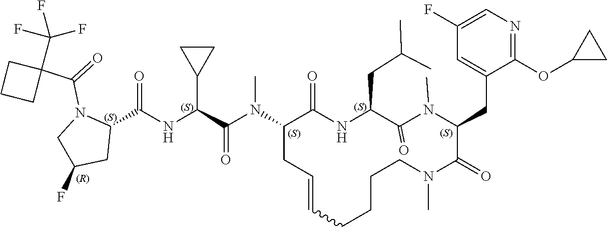 |
| 134 | 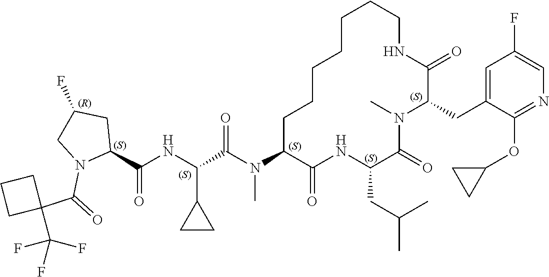 |
| 135 | 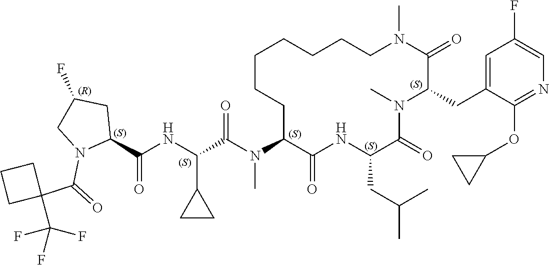 |
| 136- P1/ 136- P2 | 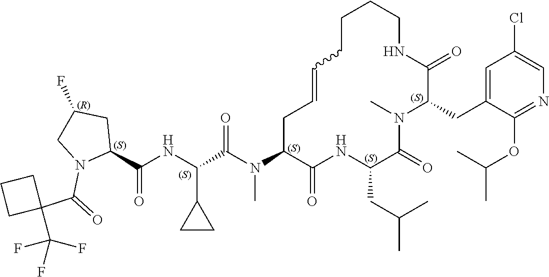 |
| 137 | 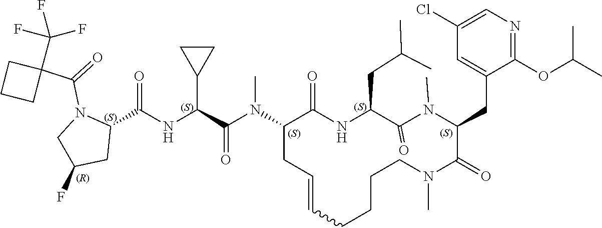 |
| 138 | 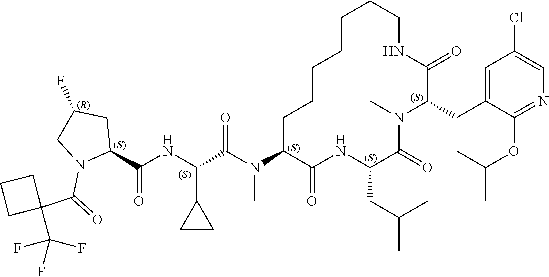 |
| 139 | 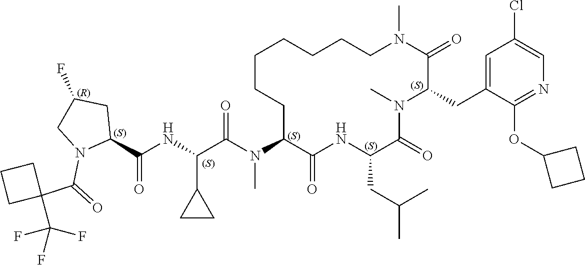 |
| 140 | 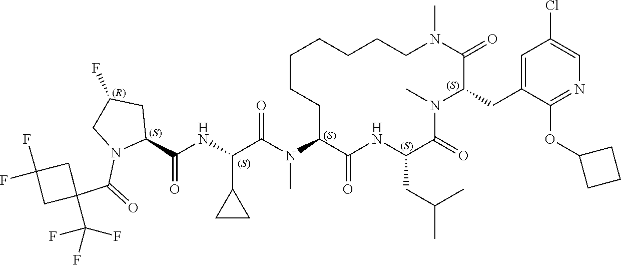 |
| 141 | 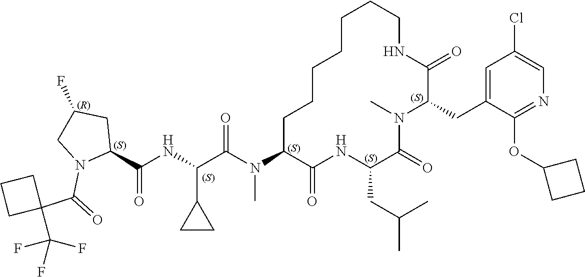 |
| 142 | 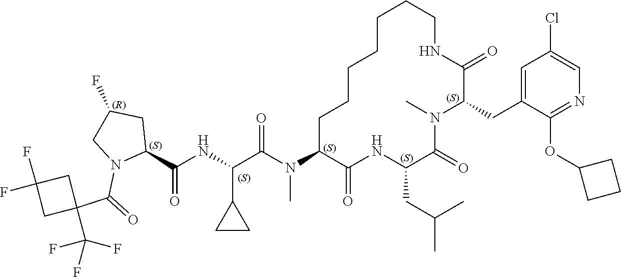 |
| 143 | 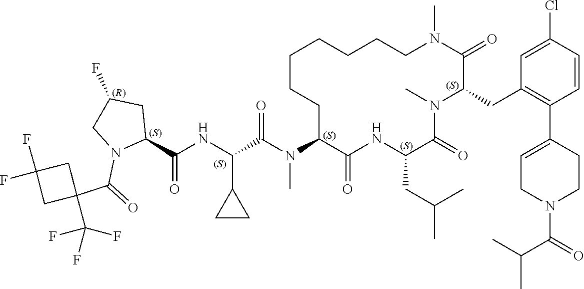 |
| 144 | 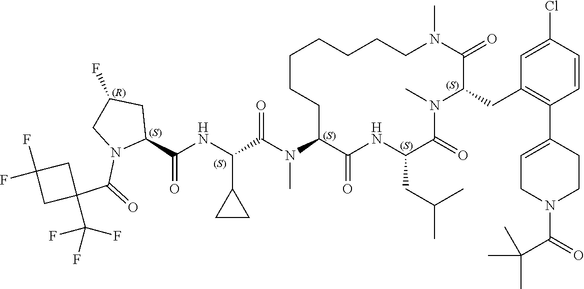 |
| 145 | 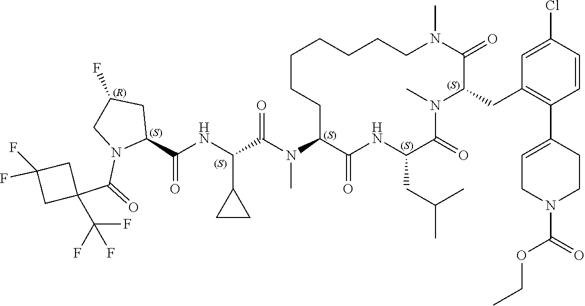 |
| 146 | 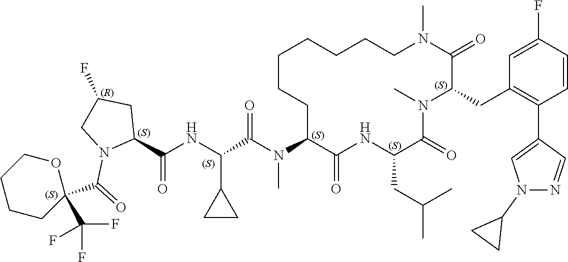 |
| 147 | 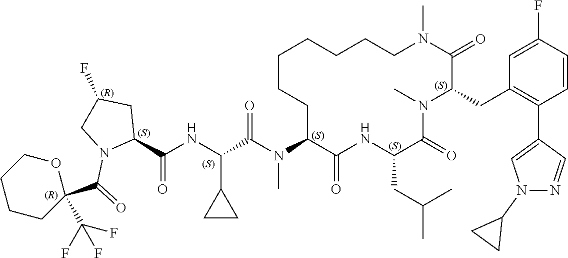 |
| 148 | 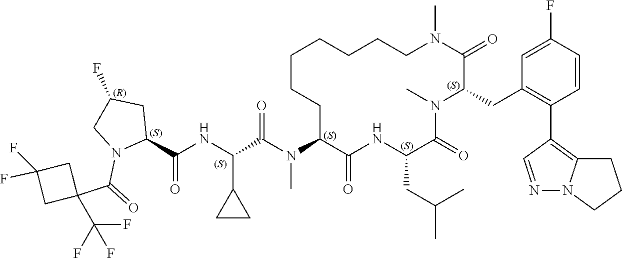 |
| 149 | 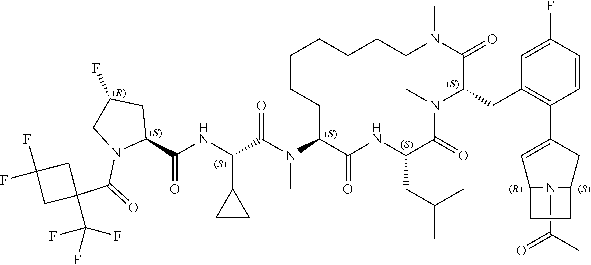 |
| 150 | 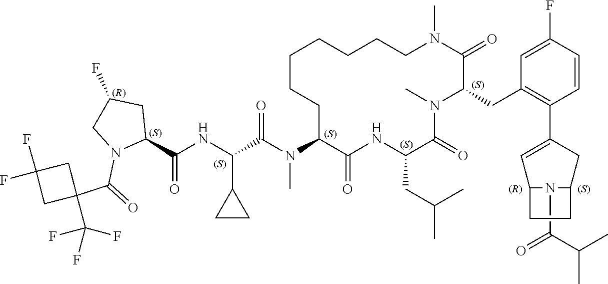 |
| 151 | 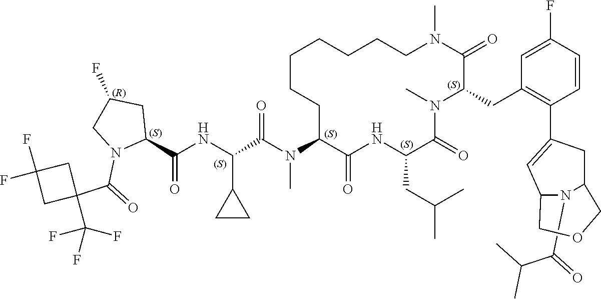 |
| 152 | 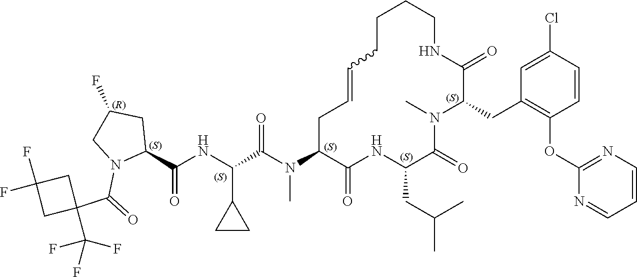 |
| 153 | 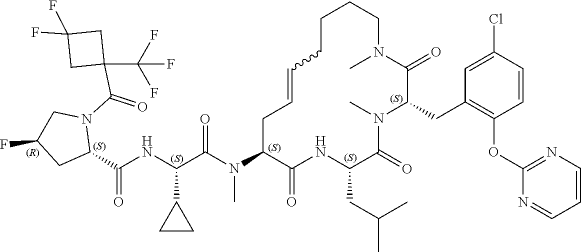 |
| 154 | 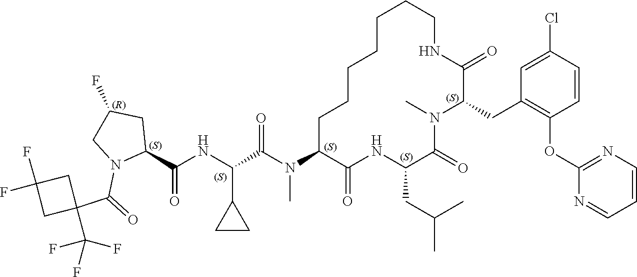 |
| 155 | 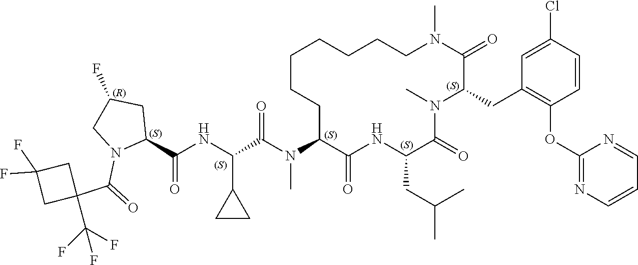 |
| 156- P1/ 156- P2 | 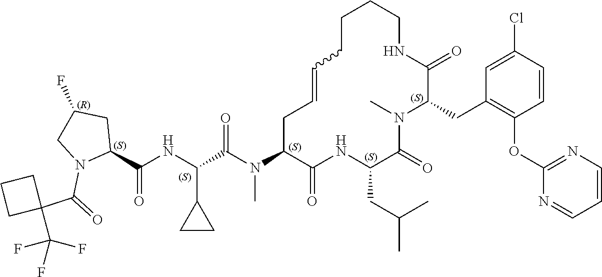 |
| 157 | 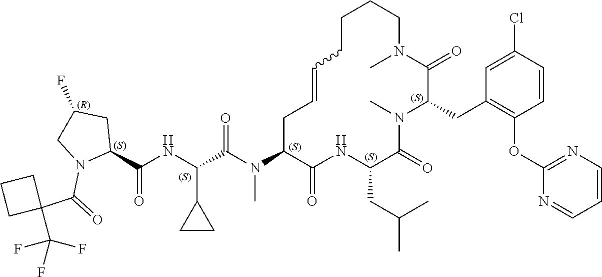 |
| 158 | 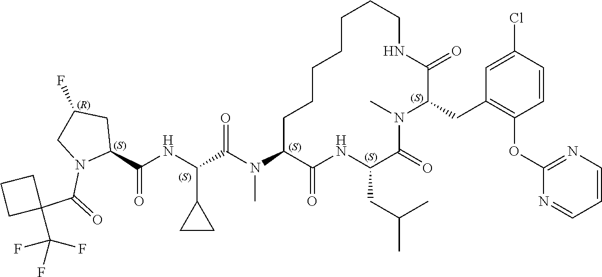 |
| 159 | 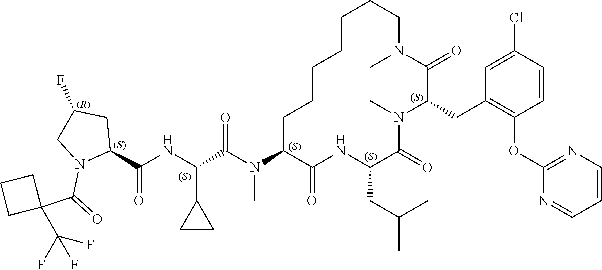 |
| 160 | 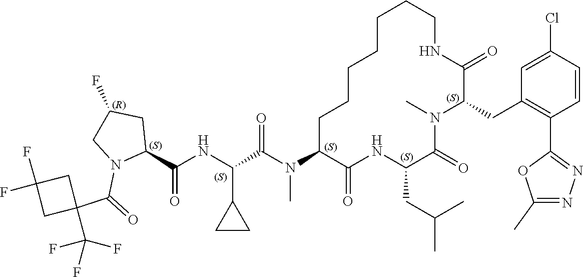 |
| 161 | 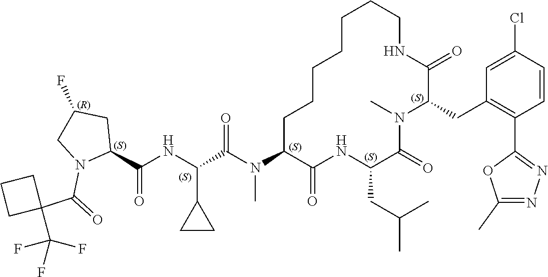 |
| 162 | 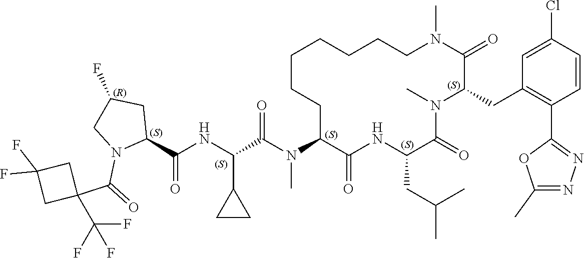 |
| 163 | 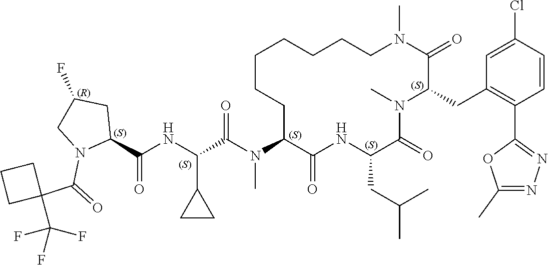 |
| 164 | 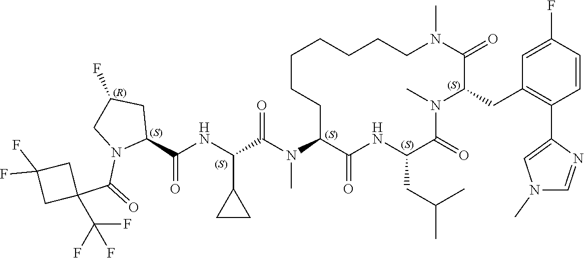 |
| 165 | 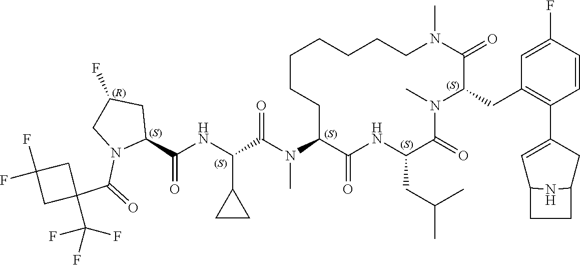 |
| 166 | 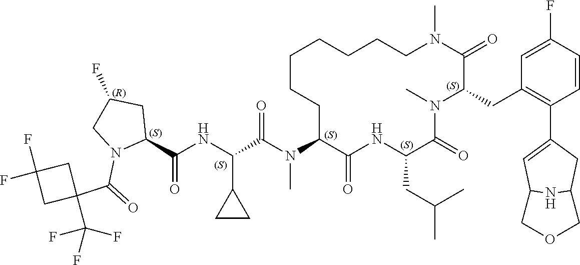 |
| 167 | 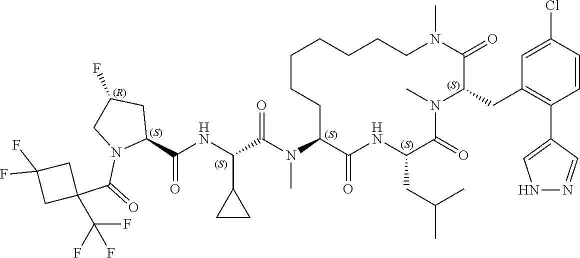 |
| 168 | 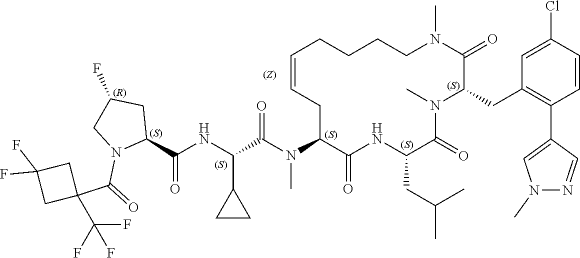 |
| 169 | 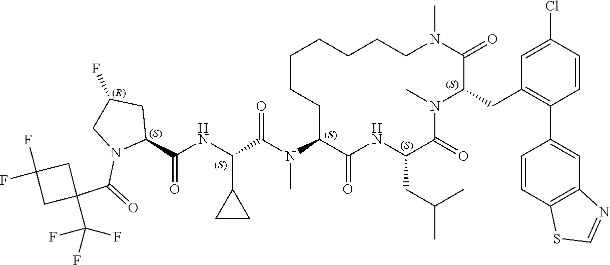 |
| 170 | 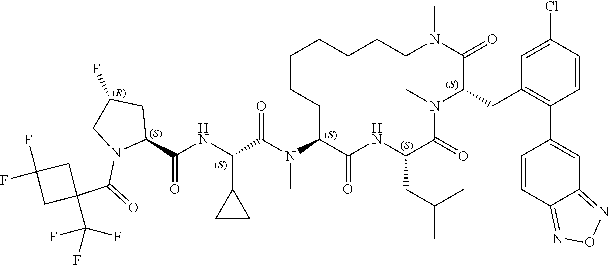 |
| 171 | 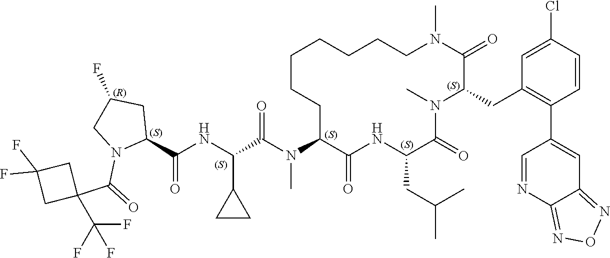 |
| 172 | 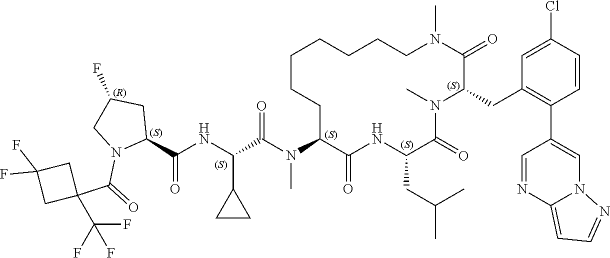 |
| 173 | 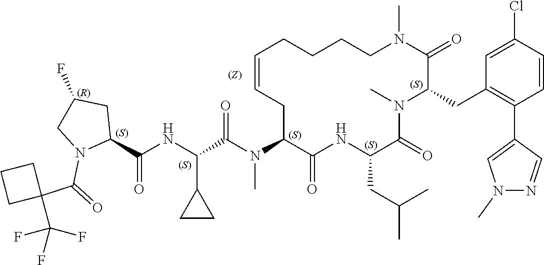 |
| 174 | 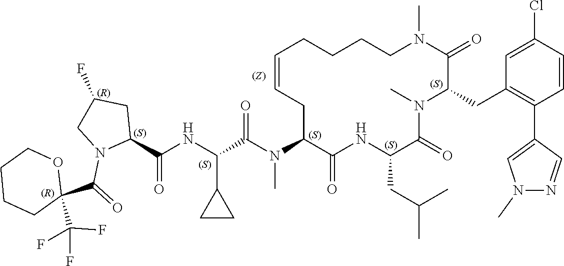 |
| 175 | 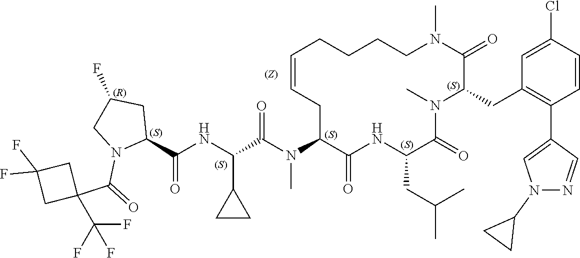 |
| 176 | 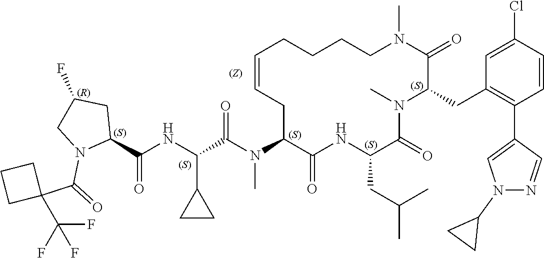 |
| 177 | 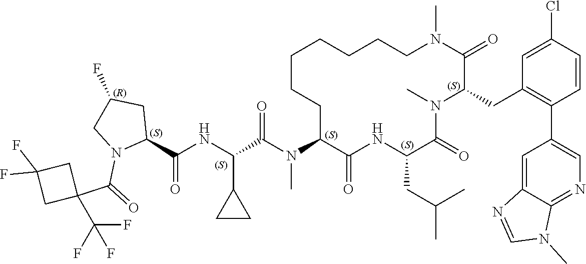 |
| 178 | 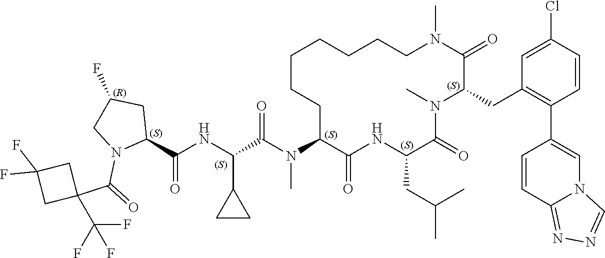 |
| 179 | 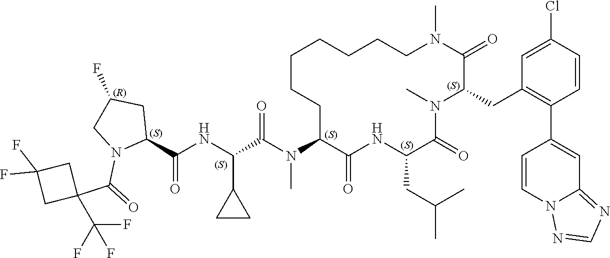 |
| 180 | 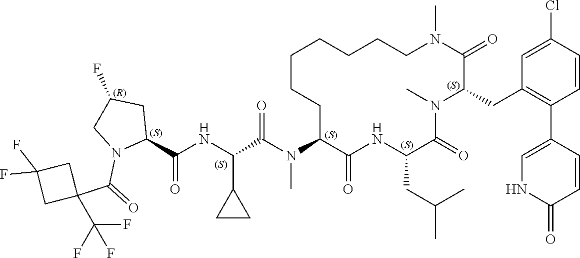 |
| 181 | 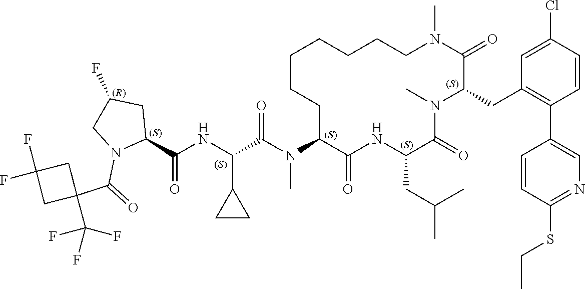 |
| 182 | 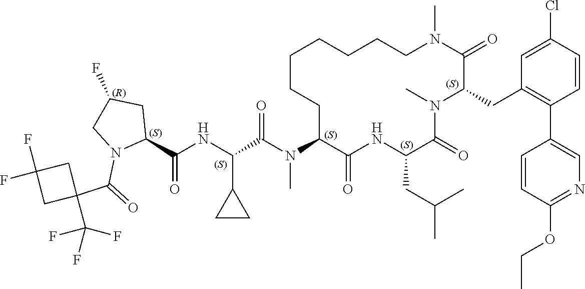 |
| 183 | 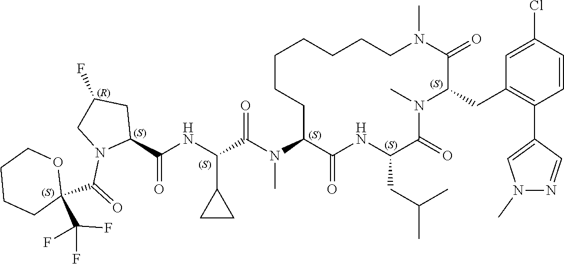 |
| 184 | 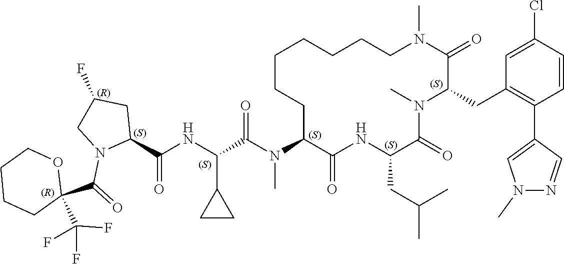 |
| 185 | 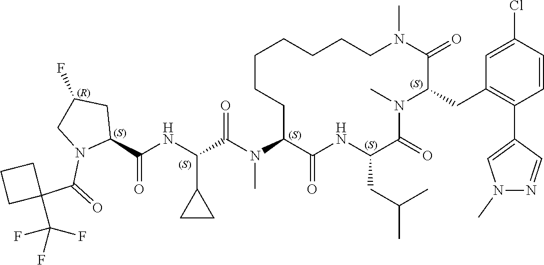 |
| 186 | 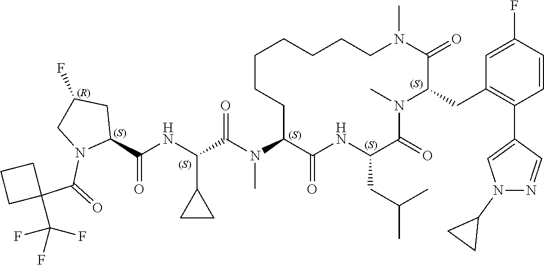 |
| 187 | 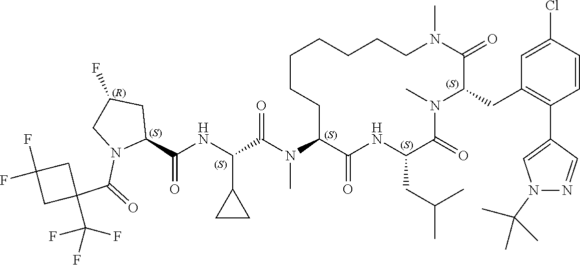 |
| 188 | 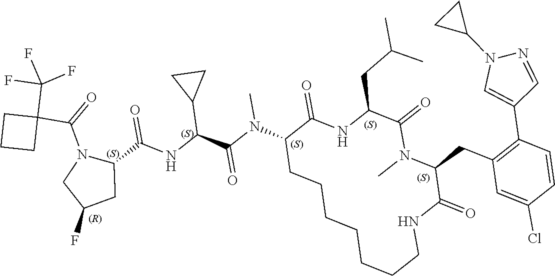 |
| 189 | 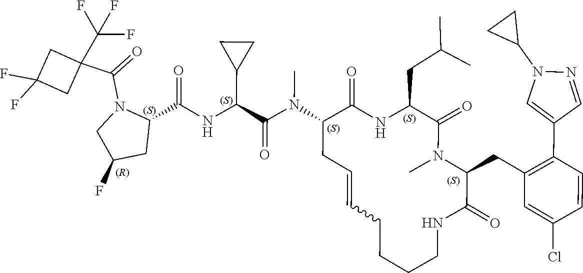 |
| 190 | 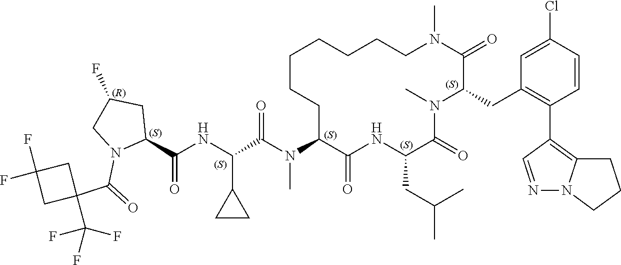 |
| 191 | 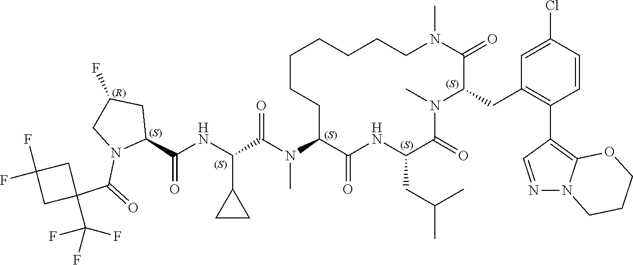 |
| 192 | 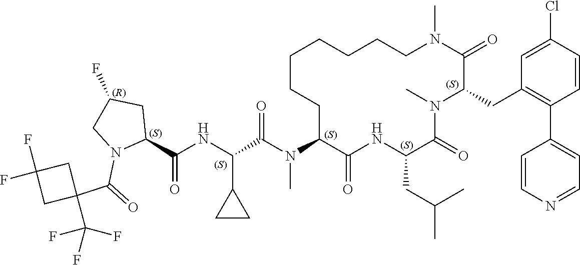 |
| 193 | 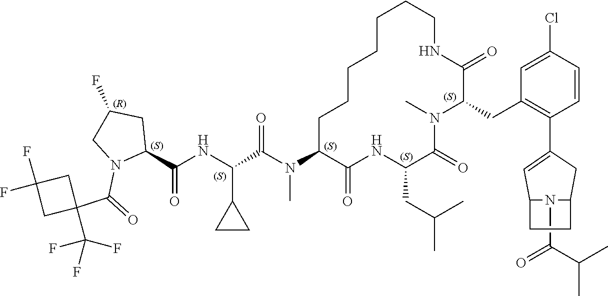 |
| 194 | 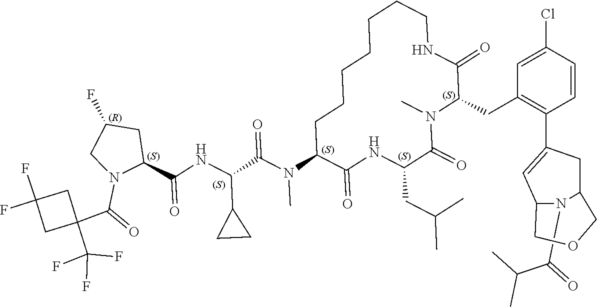 |
| 195 | 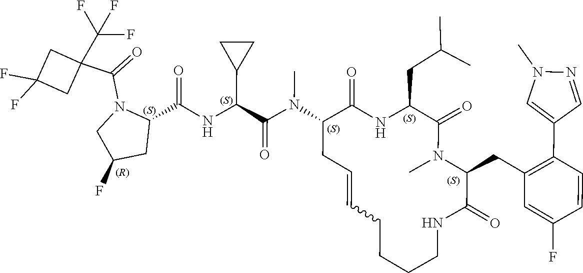 |
| 196 | 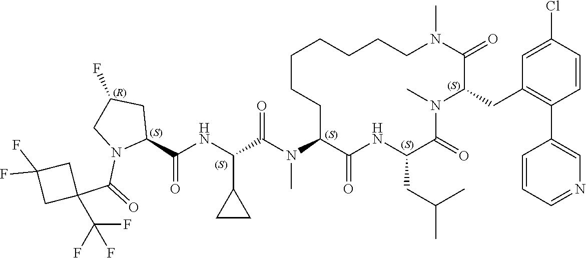 |
| 197 | 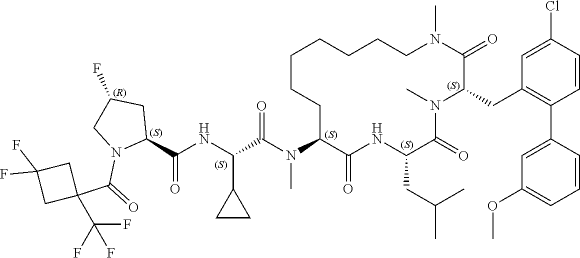 |
| 198 | 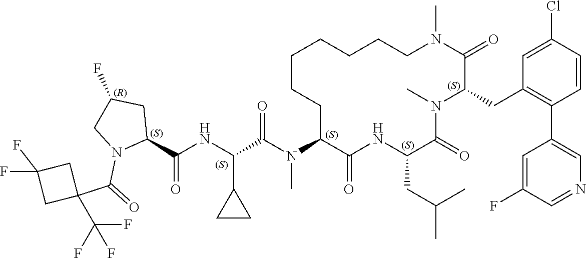 |
| 199 | 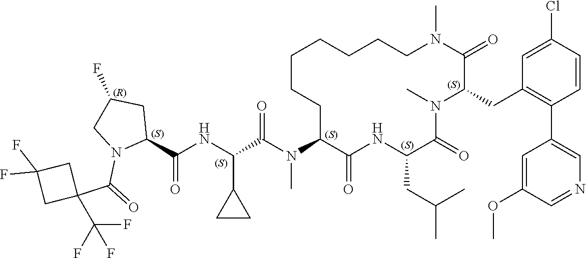 |
| 200 | 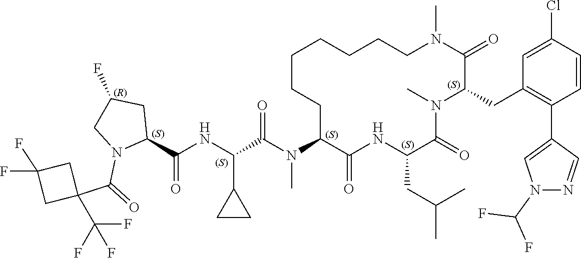 |
| 201 | 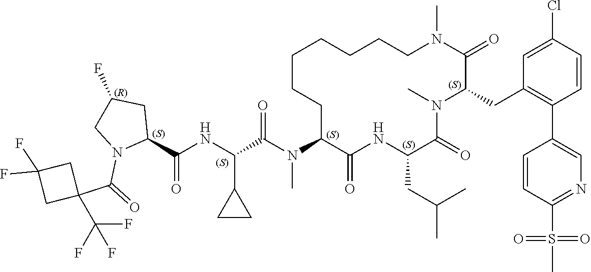 |
| 202 | 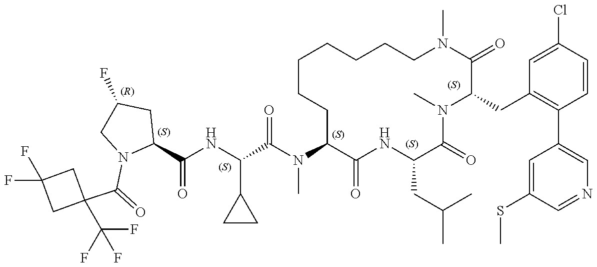 |
| 203 | 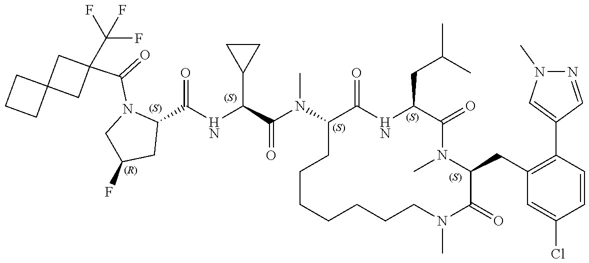 |
| 204 | 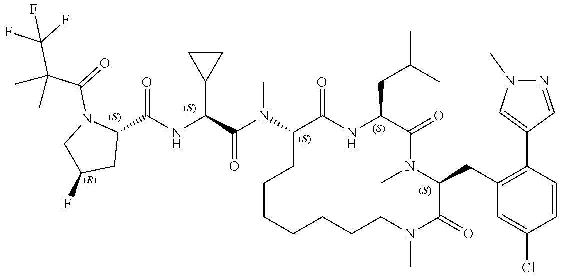 |
| 205 | 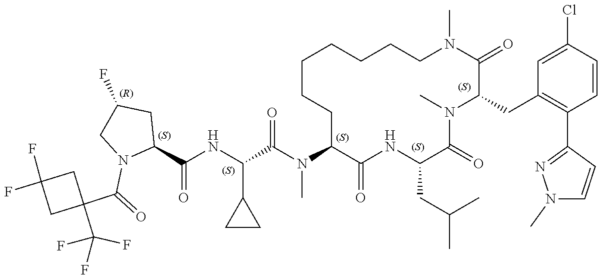 |
| 206 | 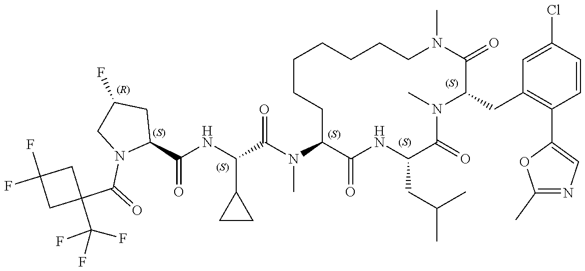 |
| 207 | 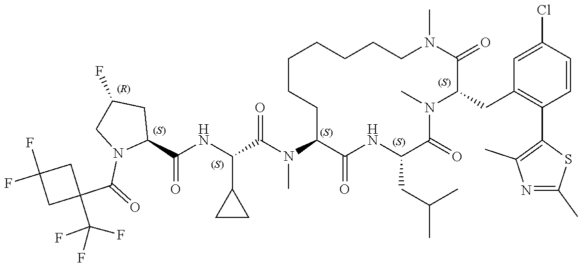 |
| 208 | 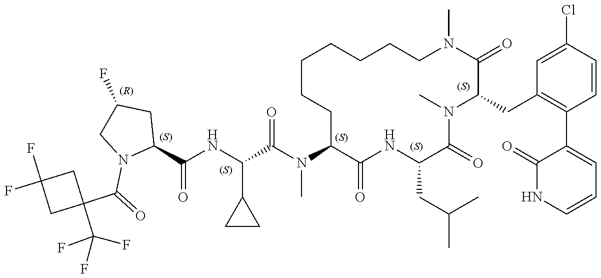 |
| 209 | 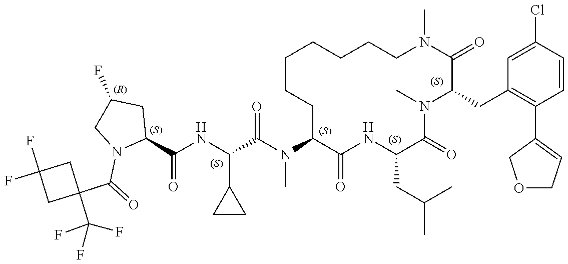 |
| 210 | 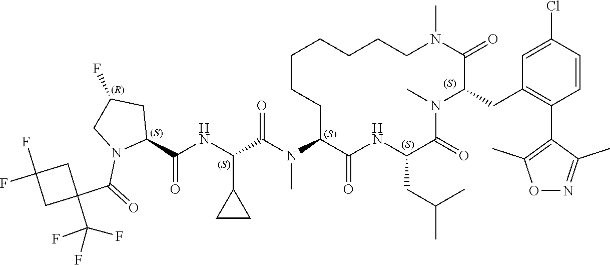 |
| 211 | 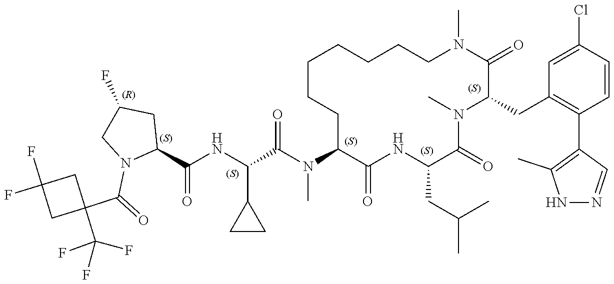 |
| 212 | 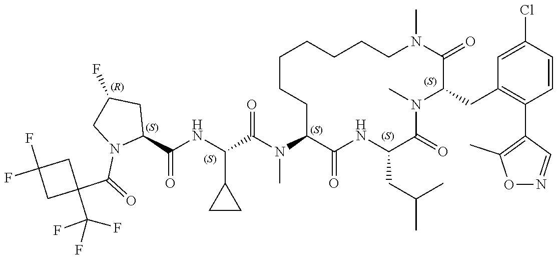 |
| 213 | 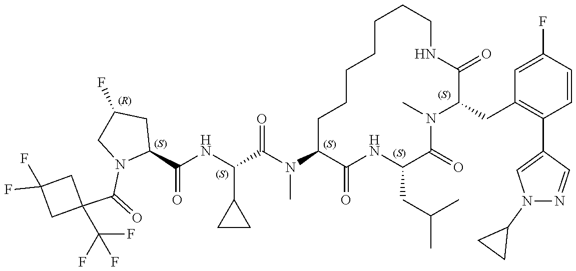 |
| 214 | 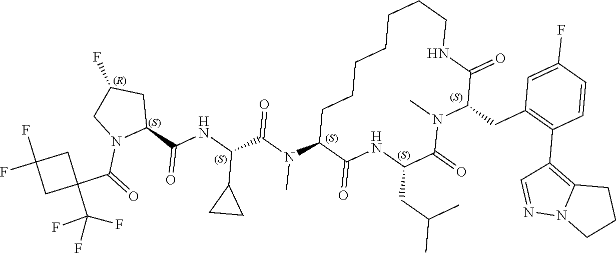 |
| 215 | 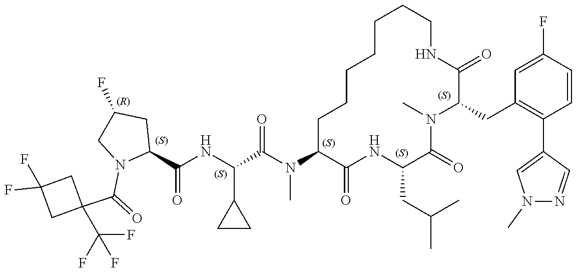 |
| 216 | 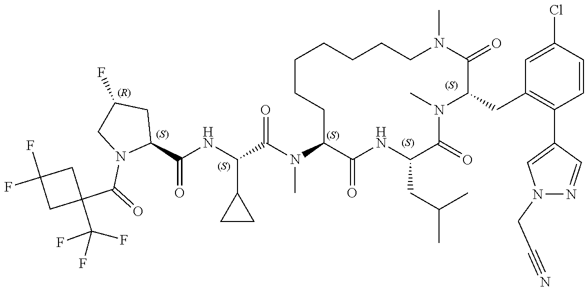 |
| 217 | 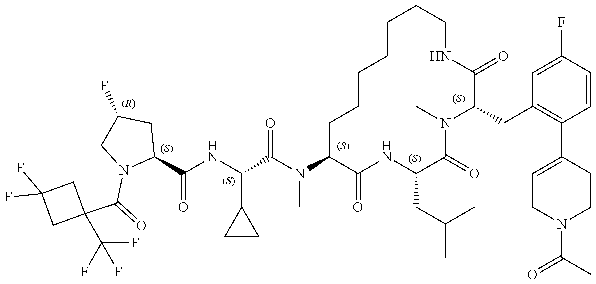 |
| 218 | 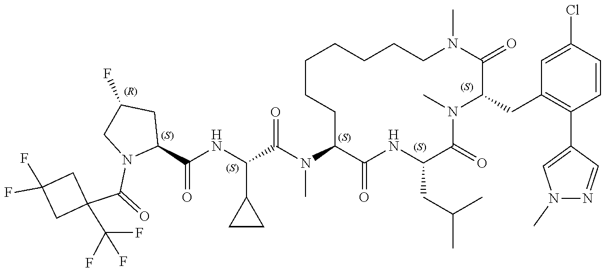 |
| 219 | 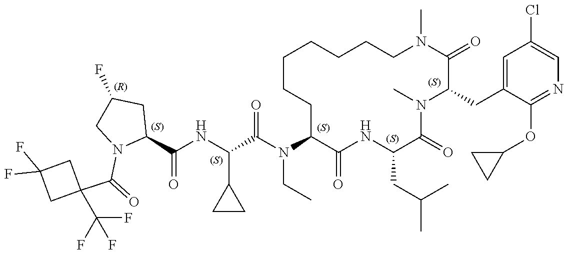 |
| 220 | 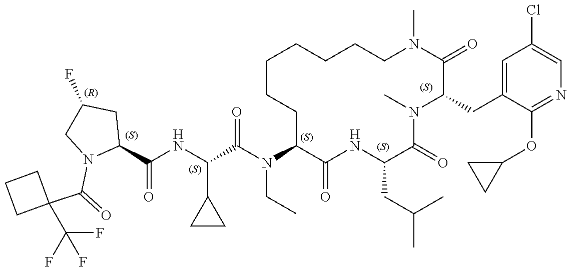 |
| 221 | 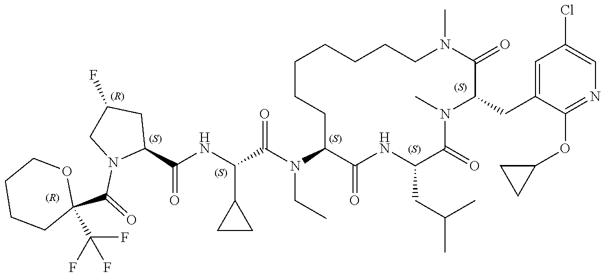 |
| 222 | 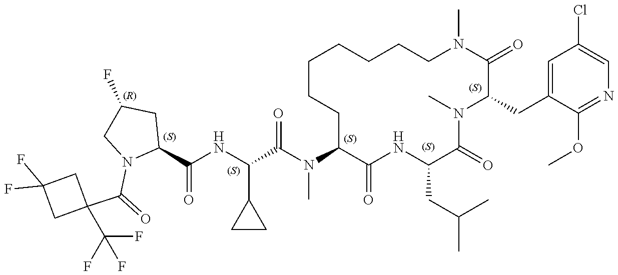 |
| 223 | 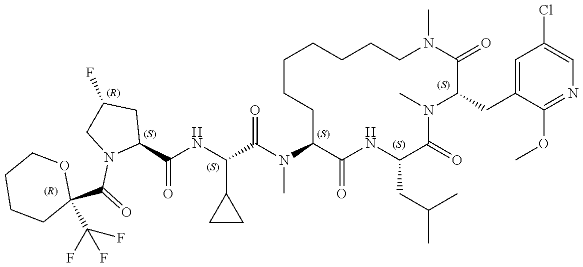 |
| 224 | 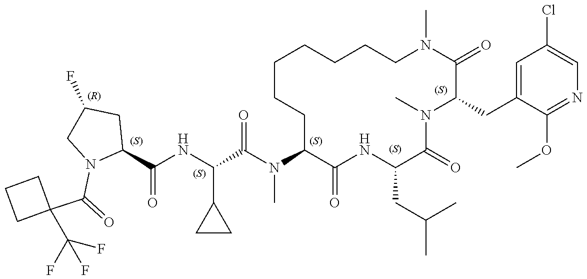 |
| 225 | 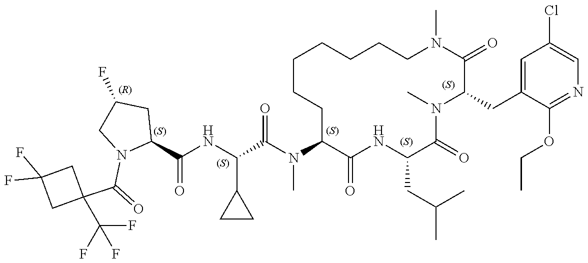 |
| 226 | 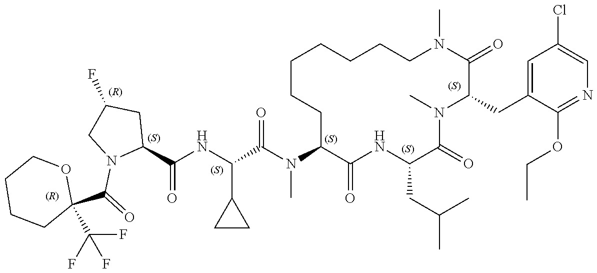 |
| 227 | 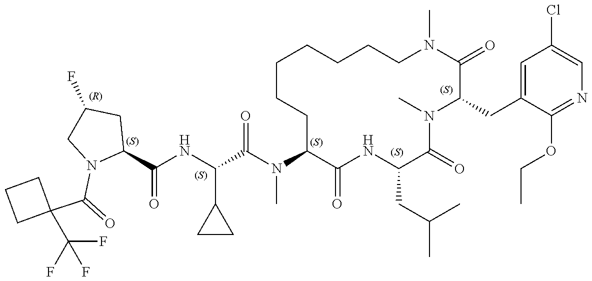 |
| 228 | 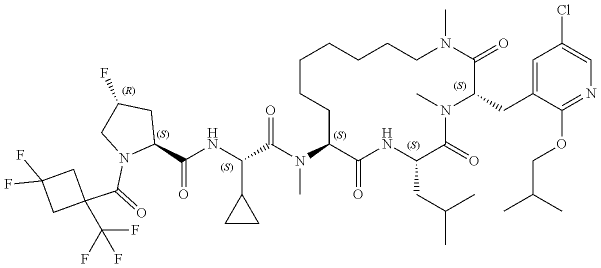 |
| 229 | 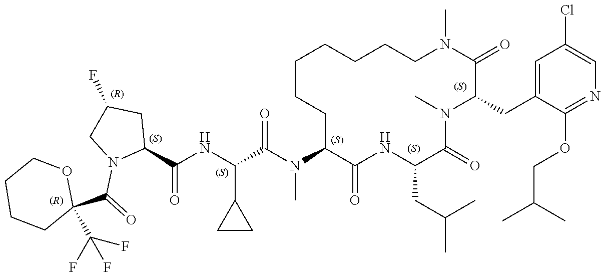 |
| 230 | 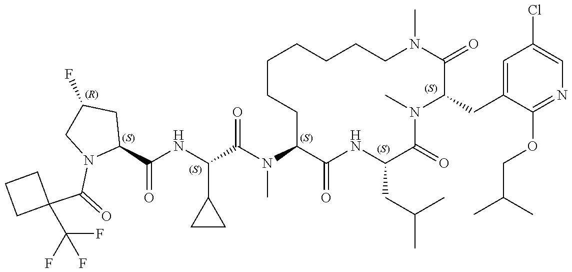 |
| 231 | 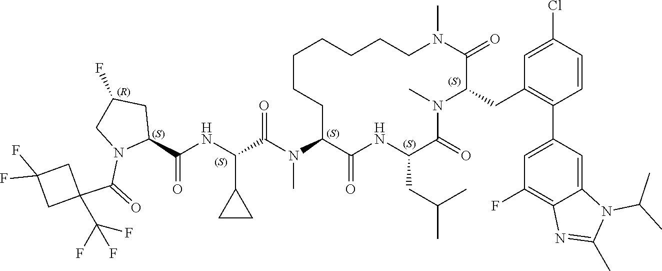 |
| 232 | 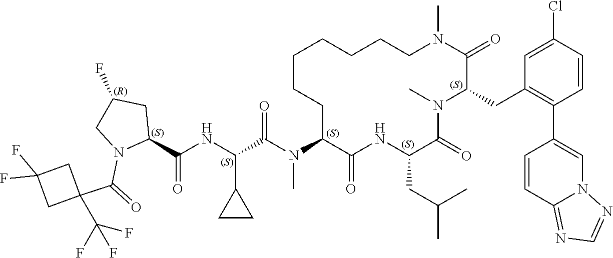 |
| 233 | 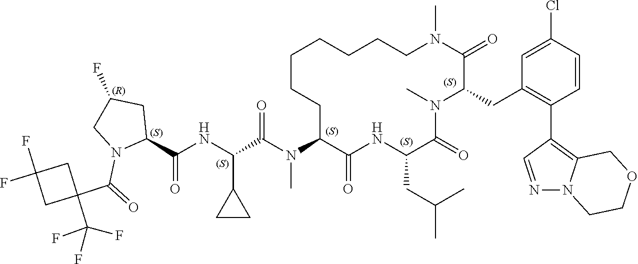 |
| 234 | 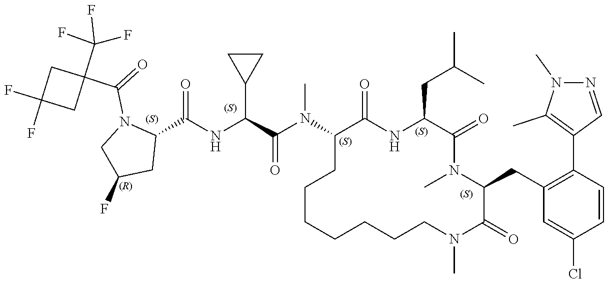 |
| 235 | 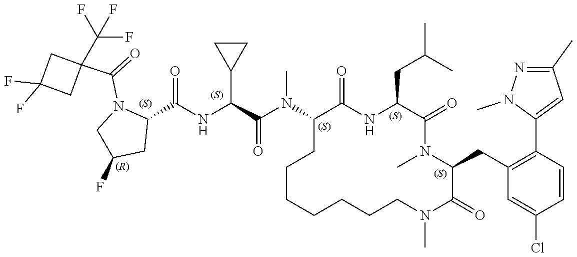 |
| 236 | 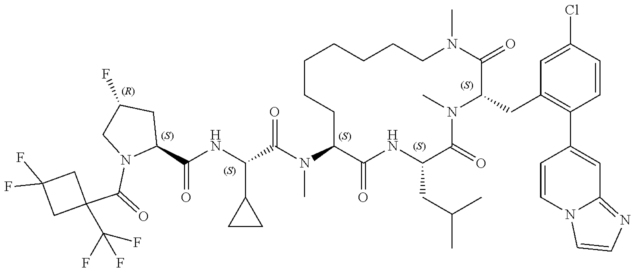 |
| 237 | 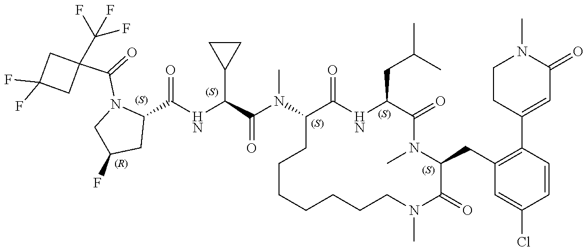 |
| 238 | 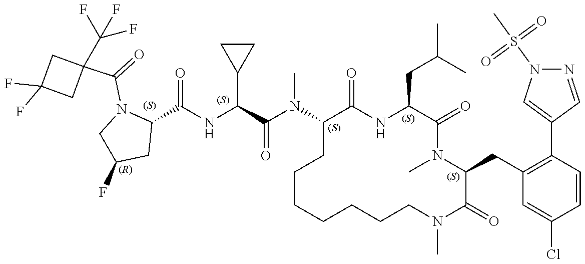 |
| 239 | 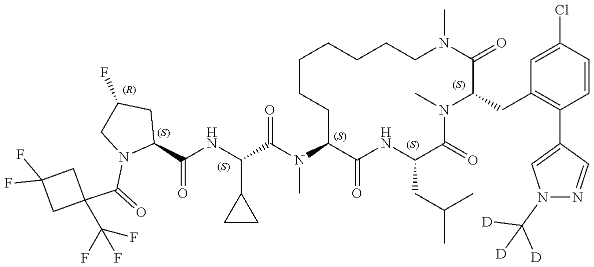 |
| 240 | 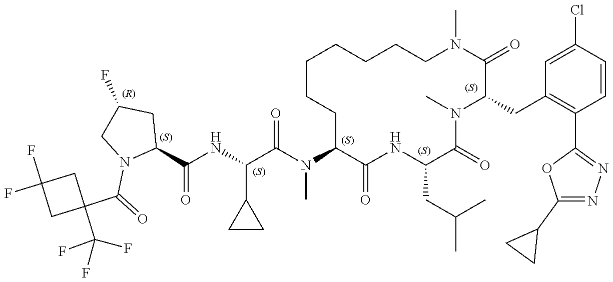 |
| 241 | 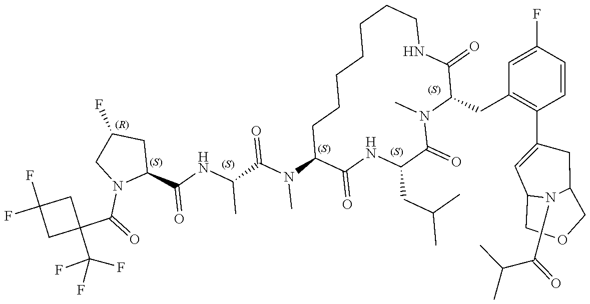 |
| 242 | 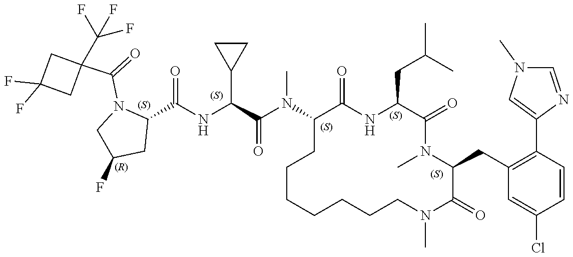 |
| 243 | 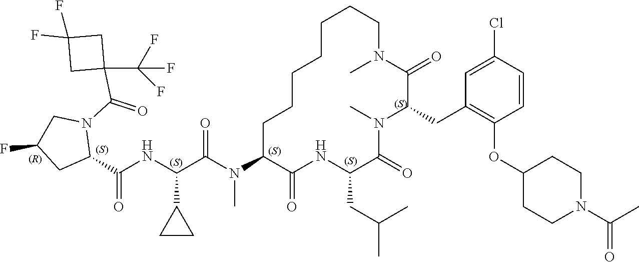 |
| 244 | 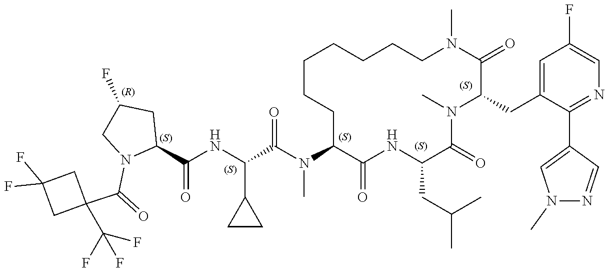 |
| 245 | 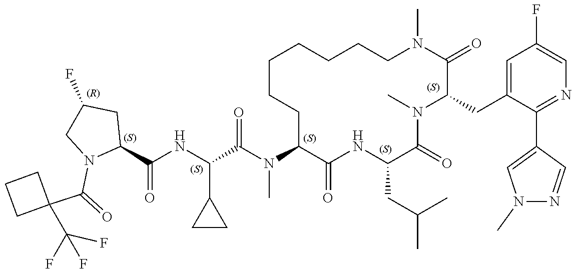 |
| 246 | 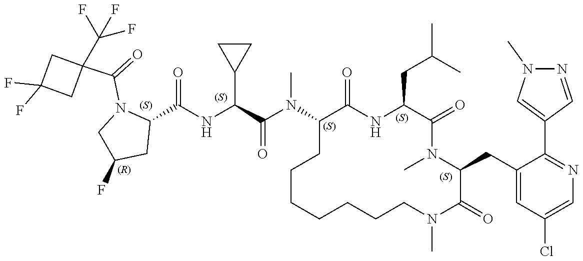 |
| 247 | 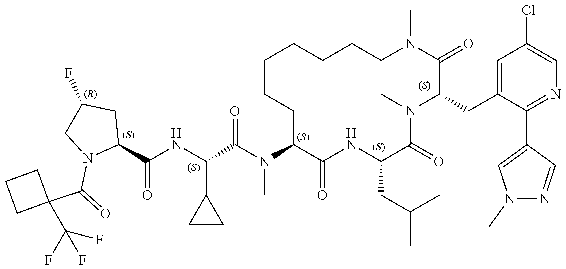 |
| 248 | 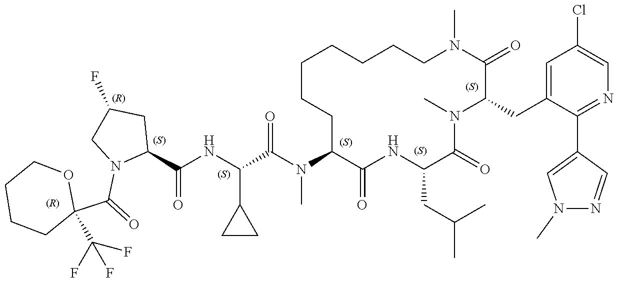 |
| 249 | 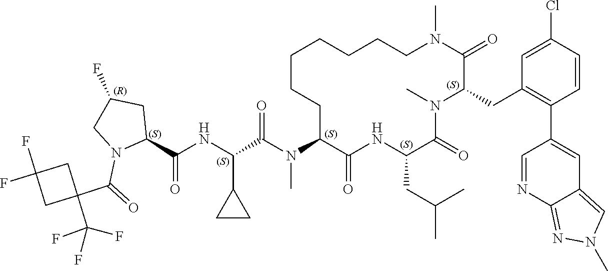 |
| 250 | 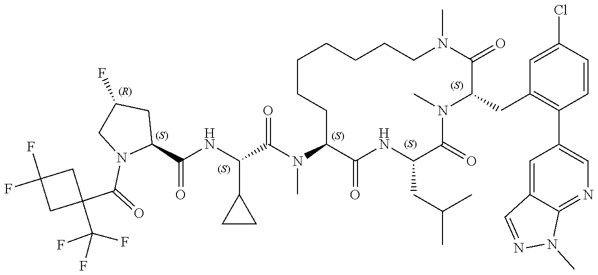 |
| 251 | 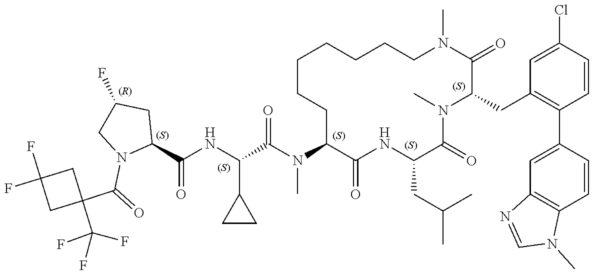 |
| 252 | 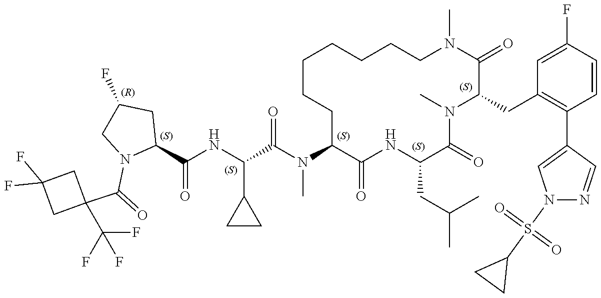 |
| 253 | 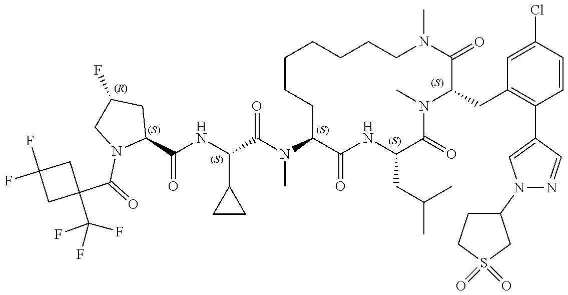 |
| 254 | 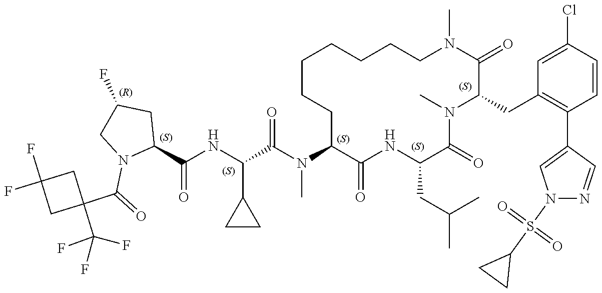 |
| 255 | 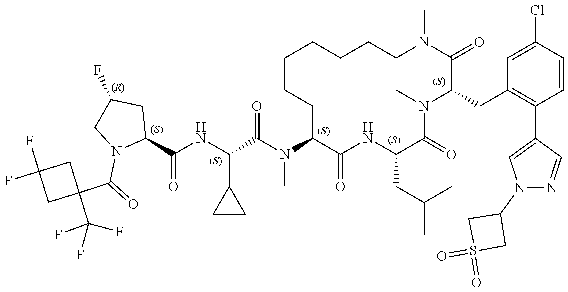 |
| 256 | 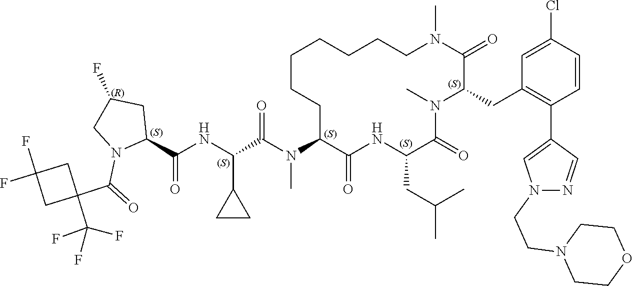 |
| 257 | 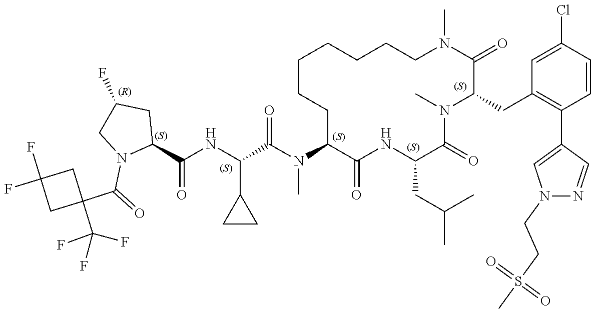 |
| 258 | 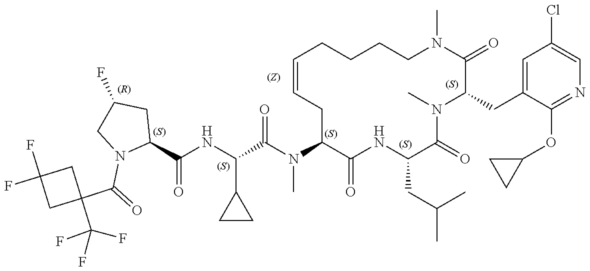 |
| 259 | 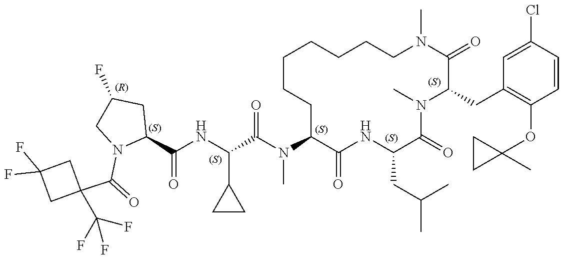 |
| 260 | 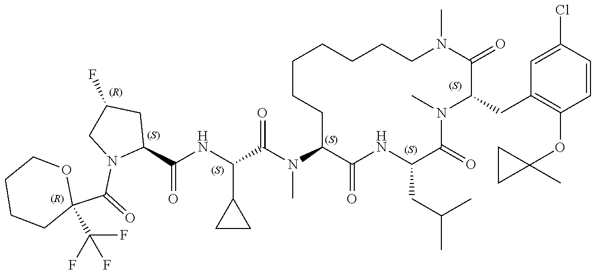 |
| 261 | 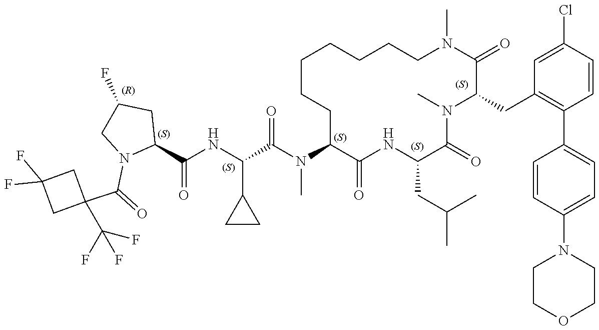 |
| 262 | 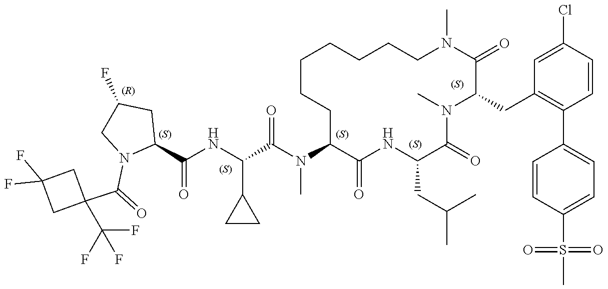 |
| 263 | 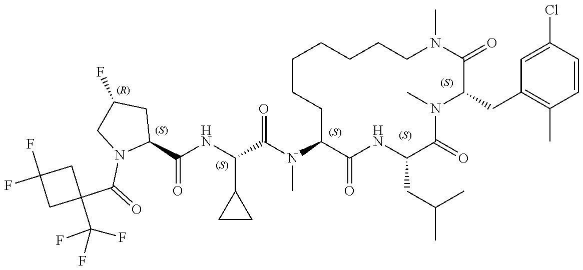 |
| 264 | 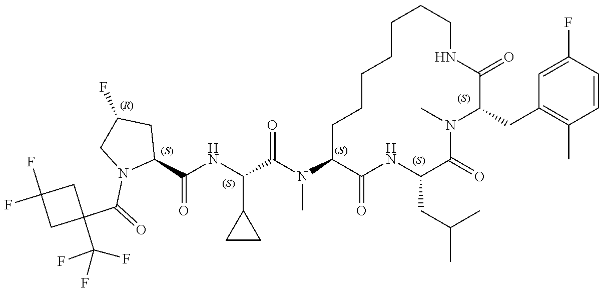 |
| 265 | 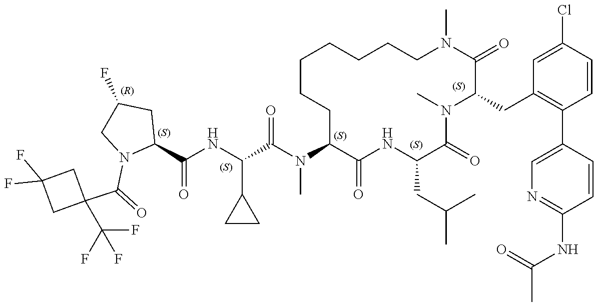 |
| 266 | 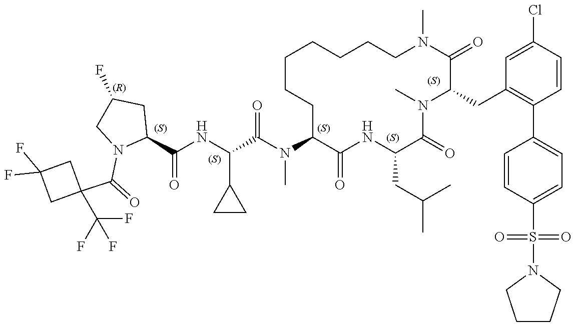 |
| 267 | 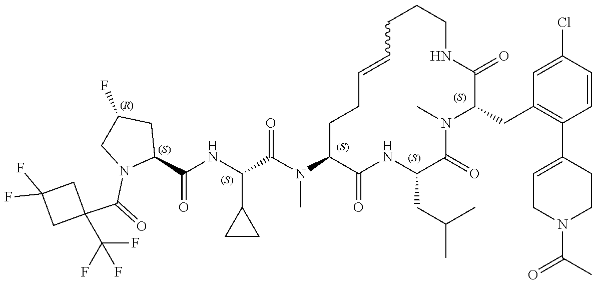 |
| 268 | 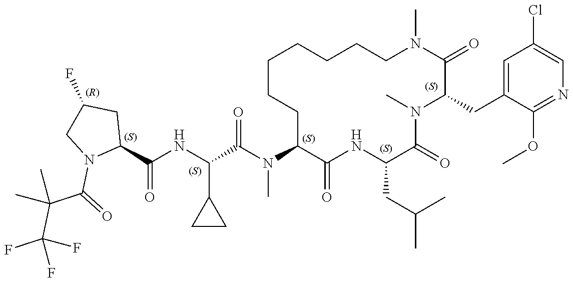 |
| 269 | 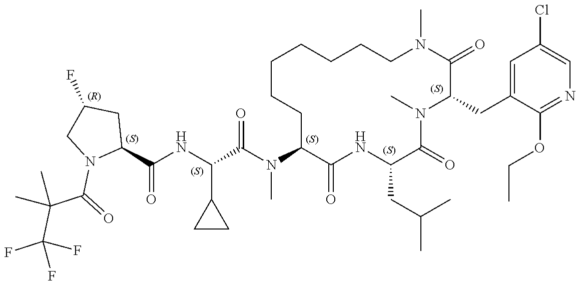 |
| 270 | 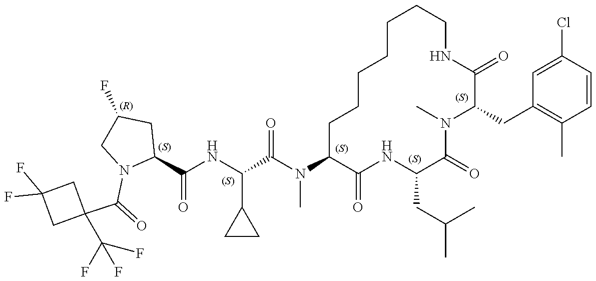 |
| 271 | 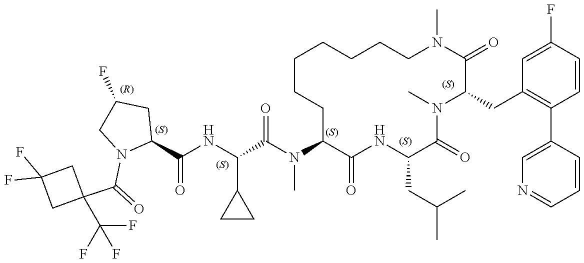 |
| 272 | 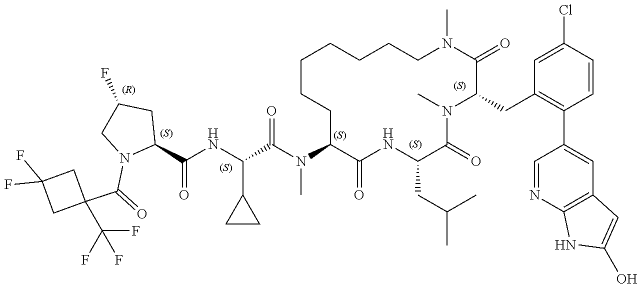 |
| 273 | 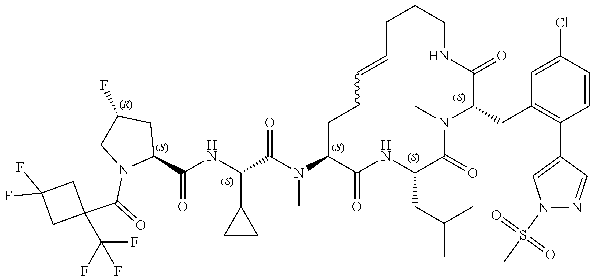 |
| 274 | 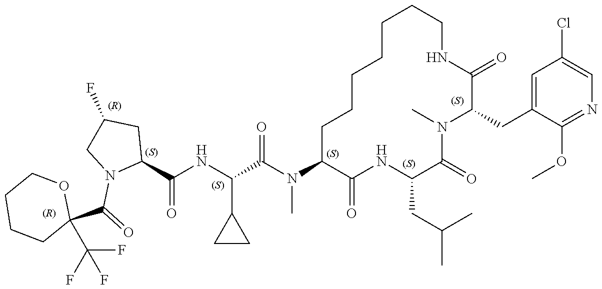 |
| 275 | 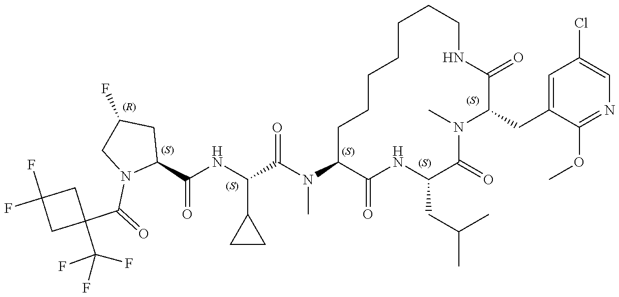 |
| 276 | 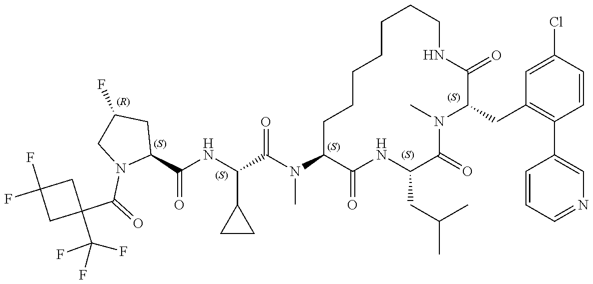 |
| 277 | 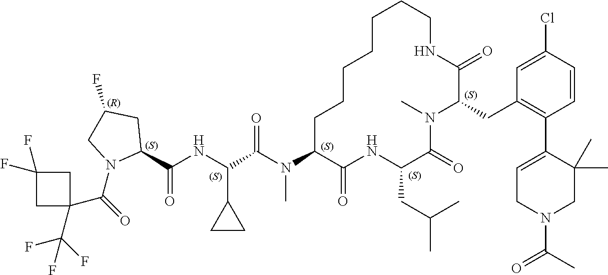 |
| 278 | 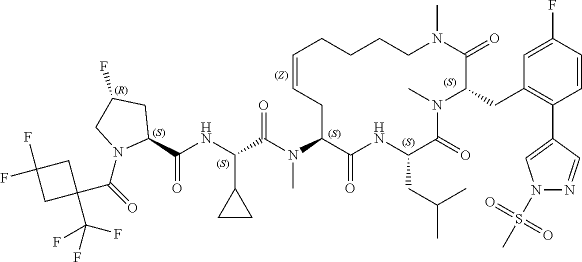 |
| 279 | 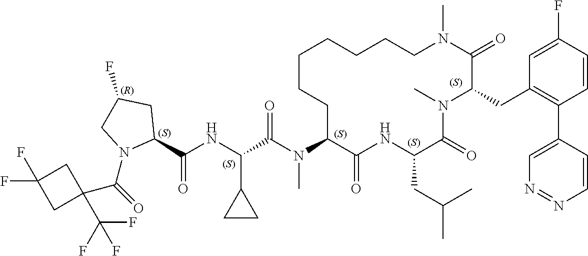 |
| 280 | 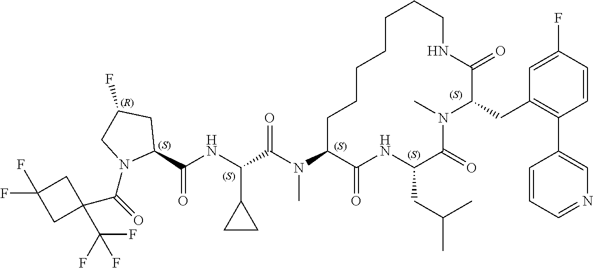 |
| 281 | 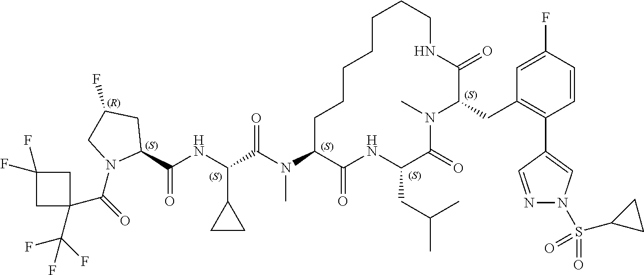 |
| 282 | 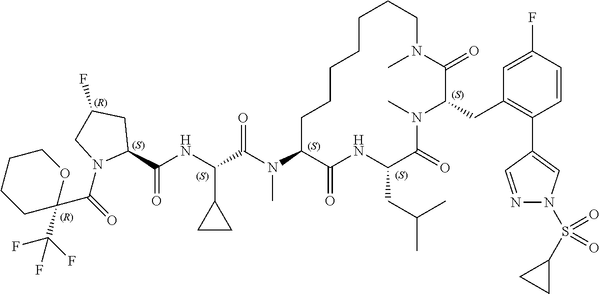 |
| 283 | 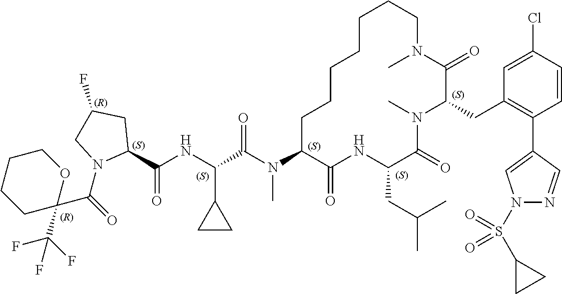 |
| 284 | 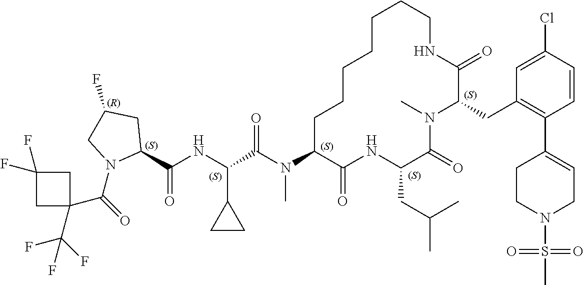 |
| 285 | 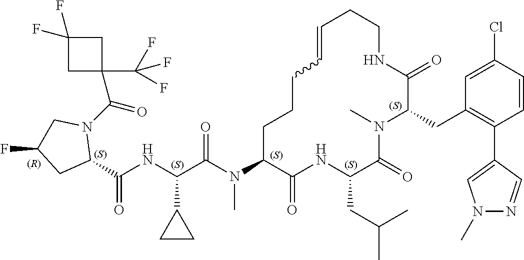 |
| 286 | 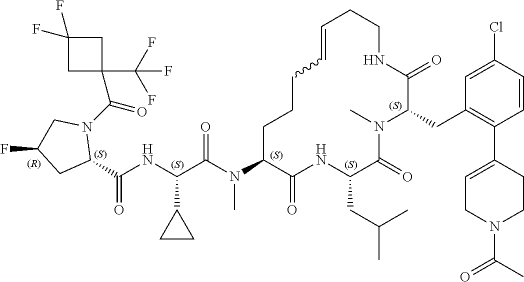 |
| 287 | 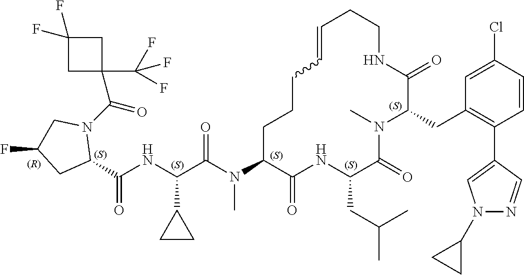 |
| 288 | 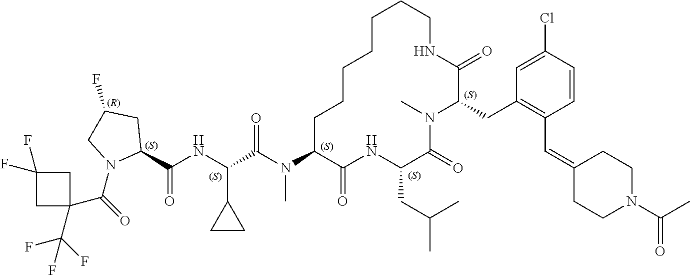 |
| 289 | 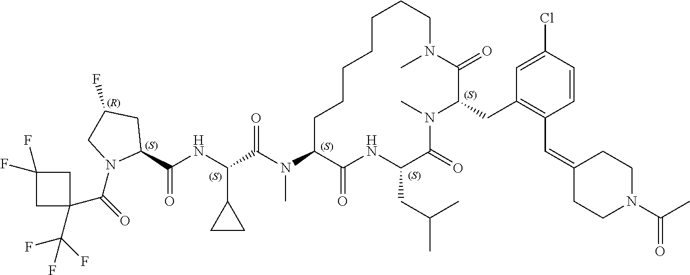 |
| 290 | 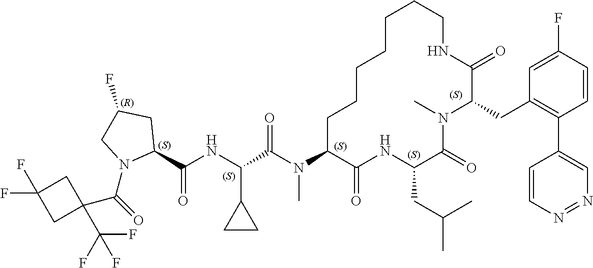 |
| 291 | 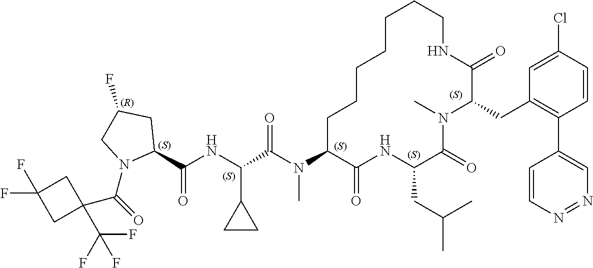 |
| 292 | 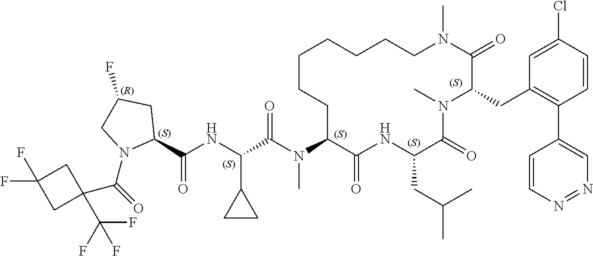 |
| 293 | 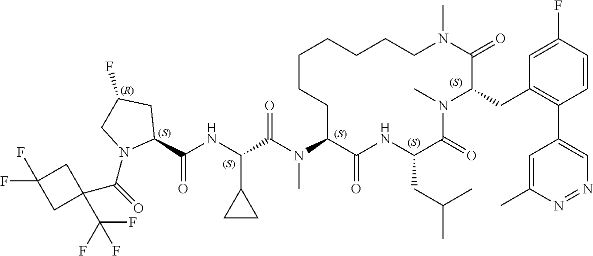 |
| 294 | 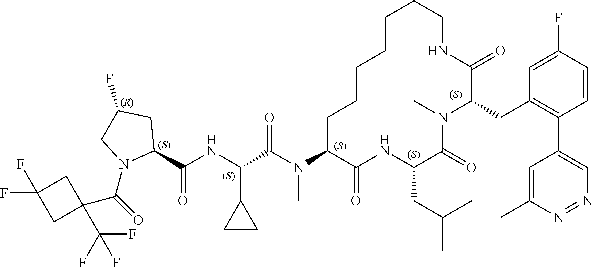 |
| 295 | 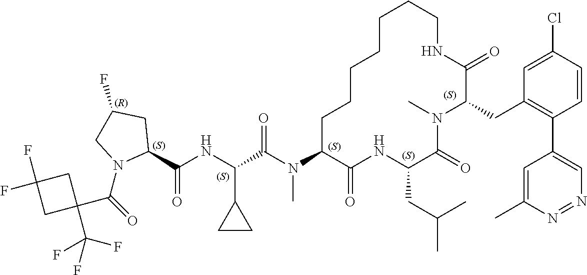 |
| 296 | 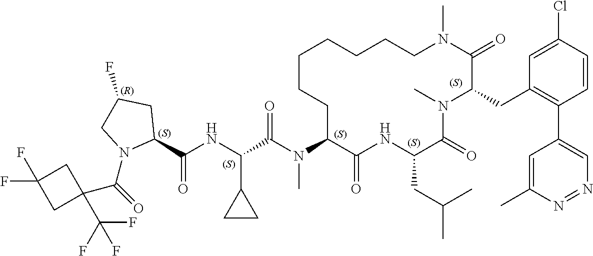 |
| 297 | 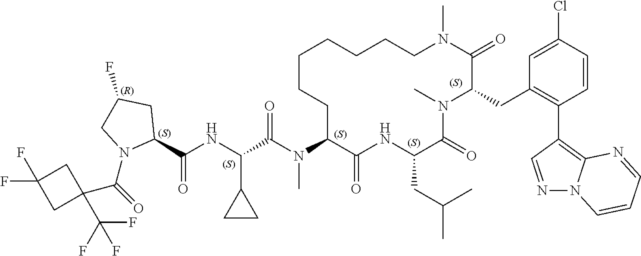 |
| 298 | 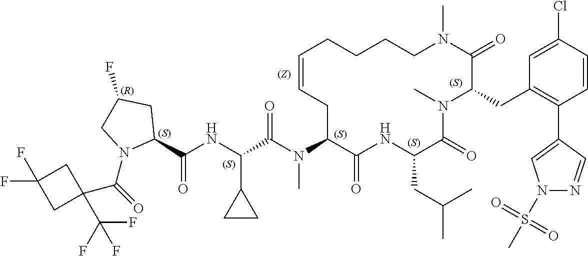 |
| 299 | 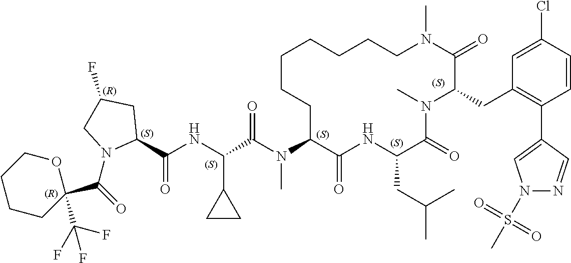 |
| 300 | 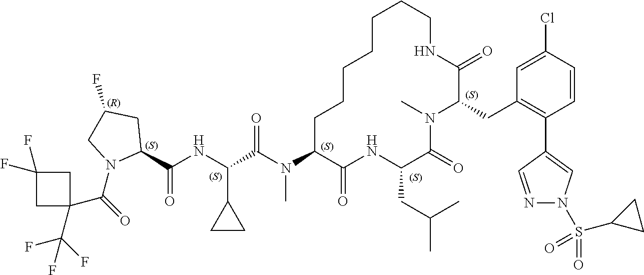 |
| 301 | 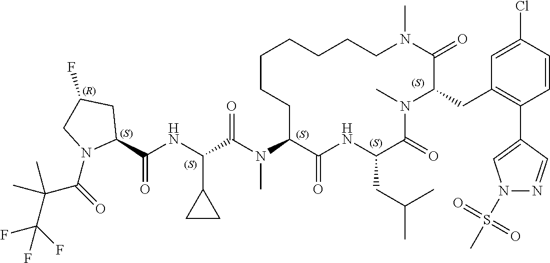 |
| 302 | 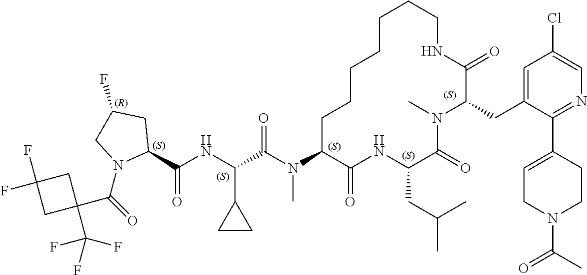 |
| 303 | 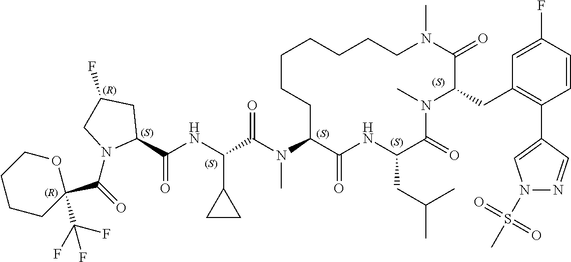 |
| 304 | 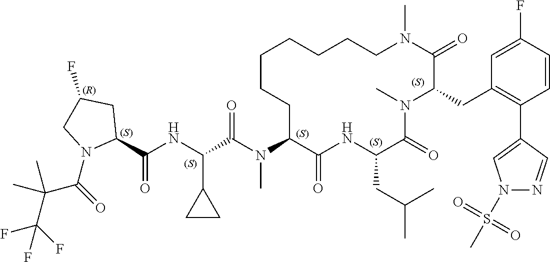 |
| 305 | 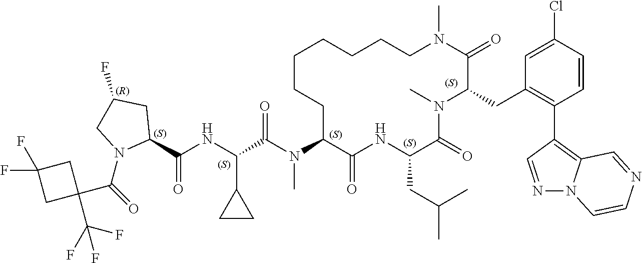 |
| 306 | 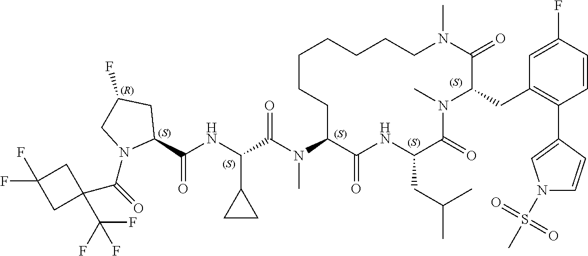 |
| 307 | 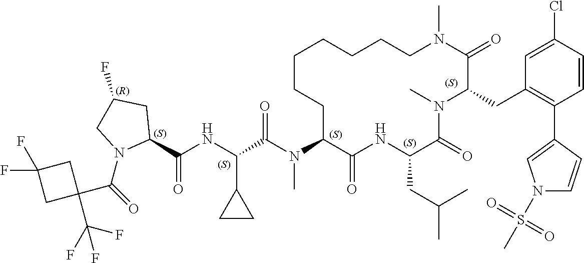 |
| 308 | 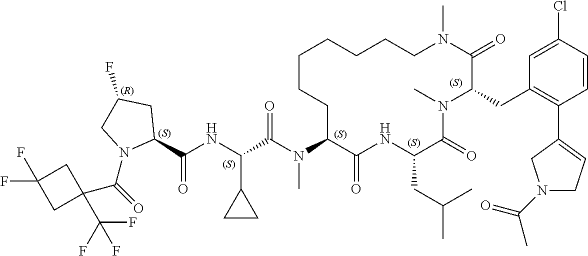 |
| 309 | 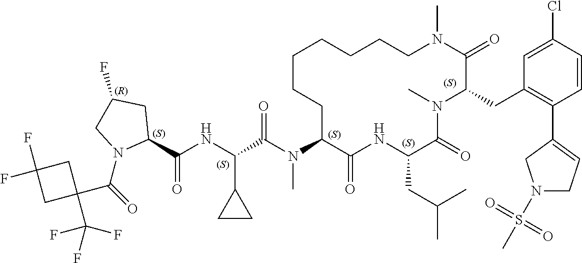 |
| 310 | 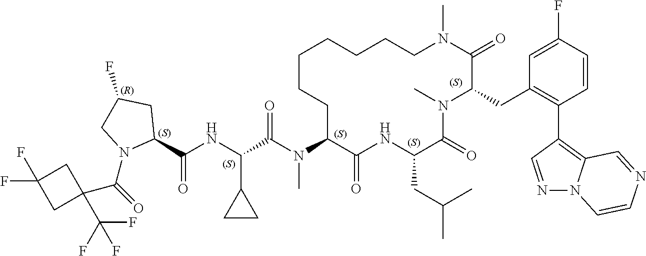 |
| 311 | 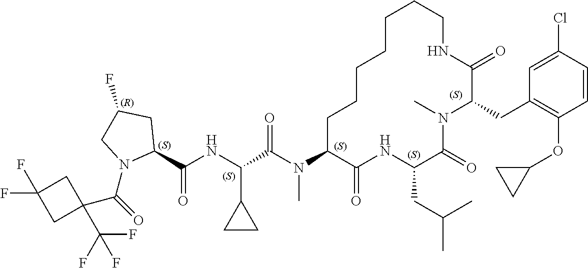 |
| 312 | 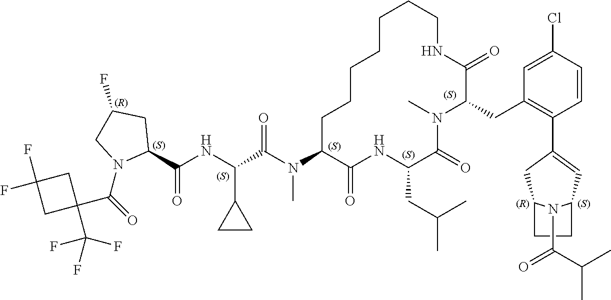 |
| 313 | 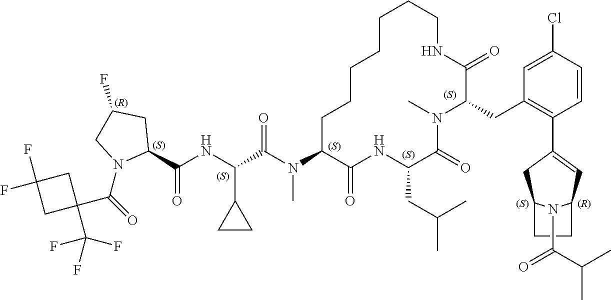 |
| 314 | 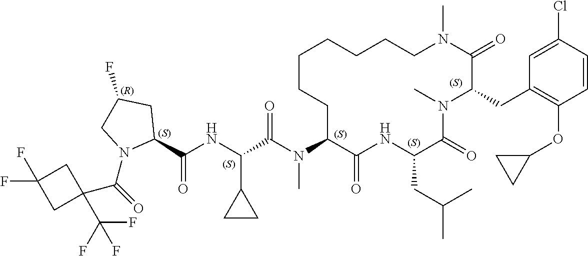 |
| 315 | 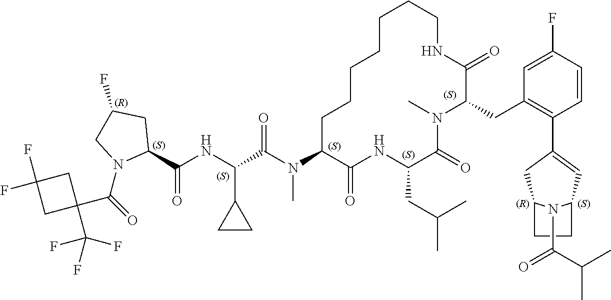 |
| 316 | 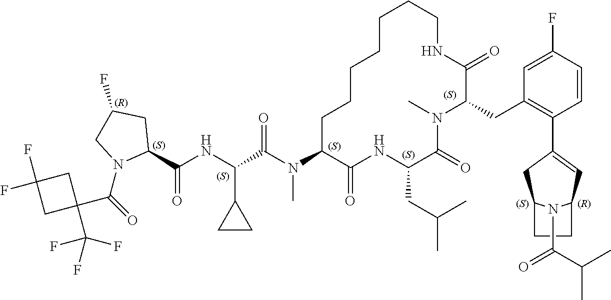 |
| 317 | 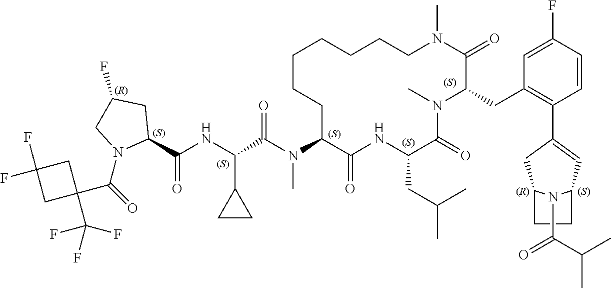 |
| 318 | 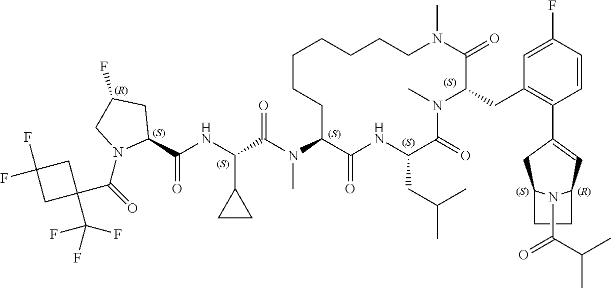 |
| 319 | 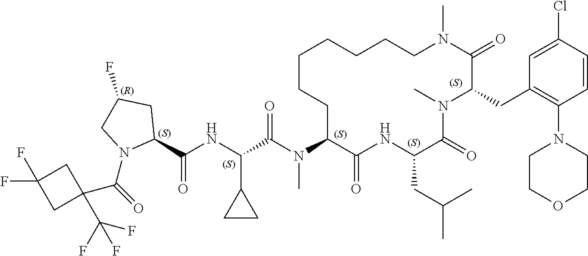 |
| 320 | 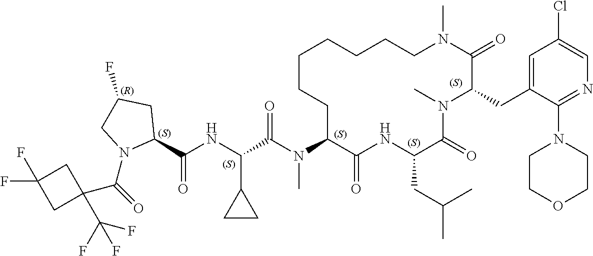 |
| 321 | 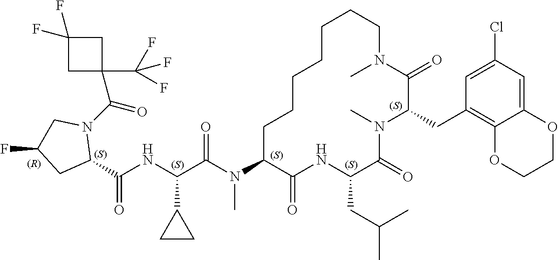 |
| 322 | 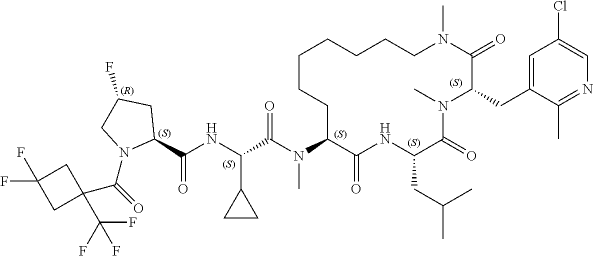 |
| 323 | 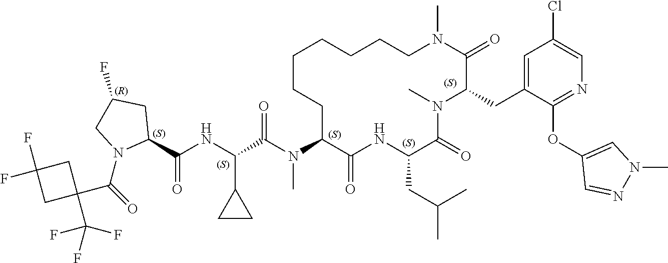 |
| 324 | 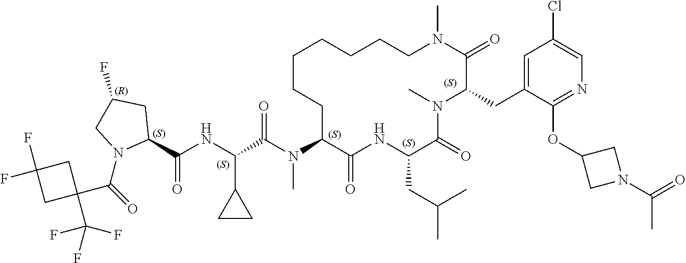 |
| 325 | 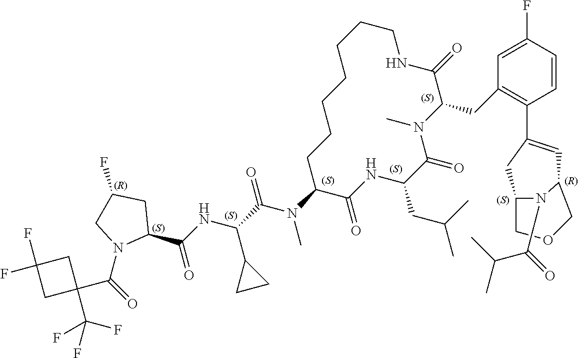 |
| 326 | 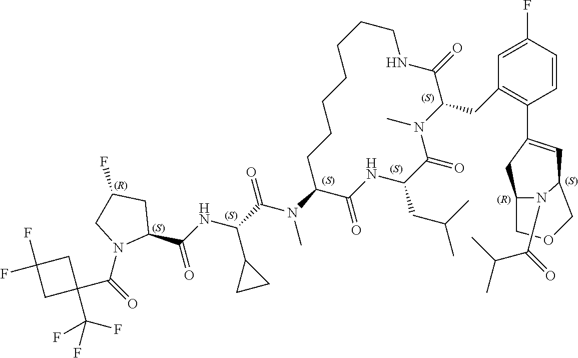 |
| 327 | 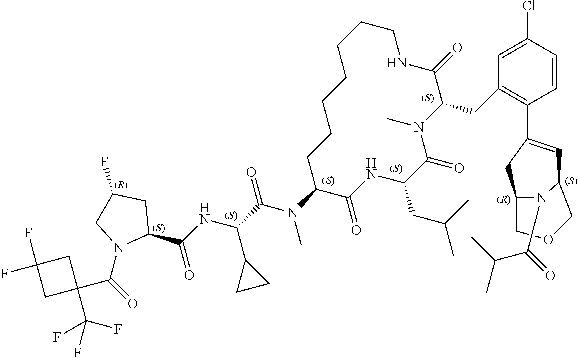 |
| 328 | 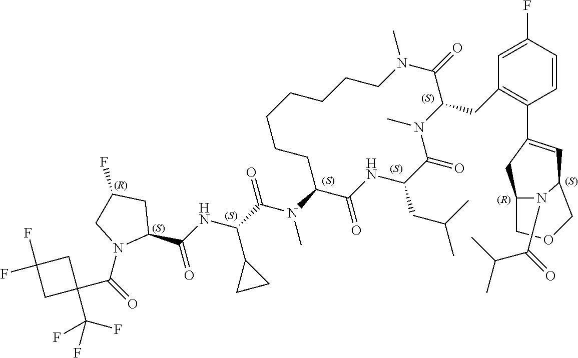 |
| 329 | 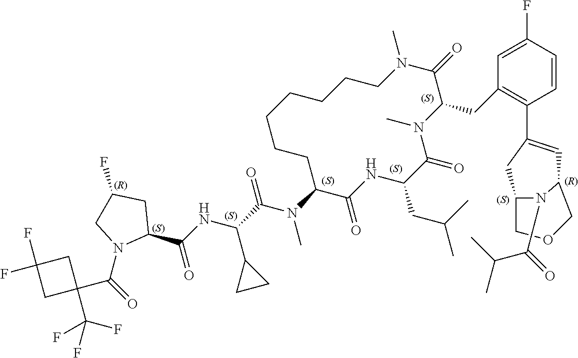 |
| 330 | 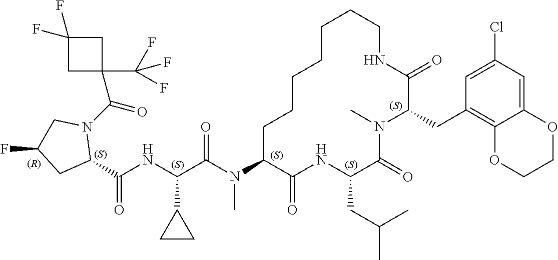 |
| 331 | 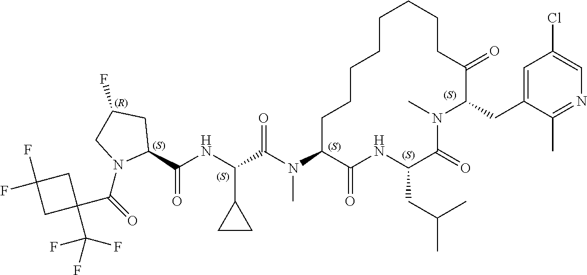 |
| 332 | 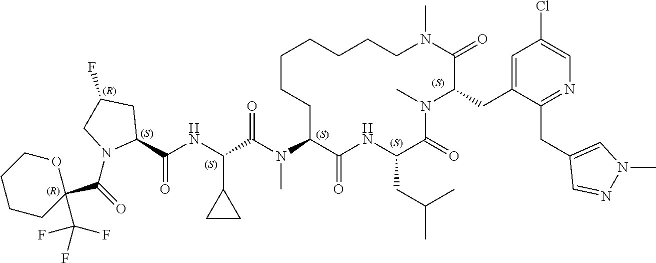 |
| 333 | 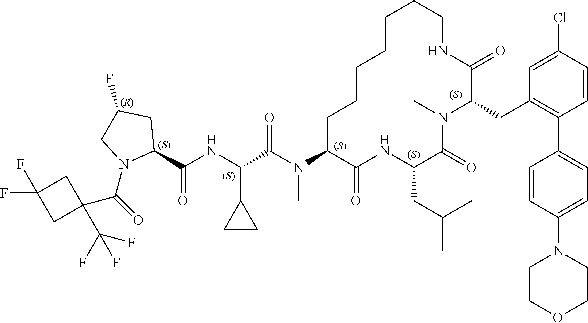 |
| 334 | 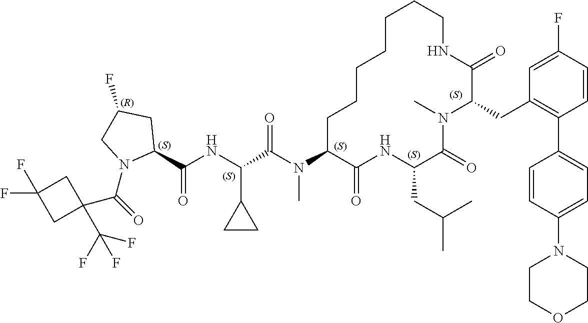 |
| 335 | 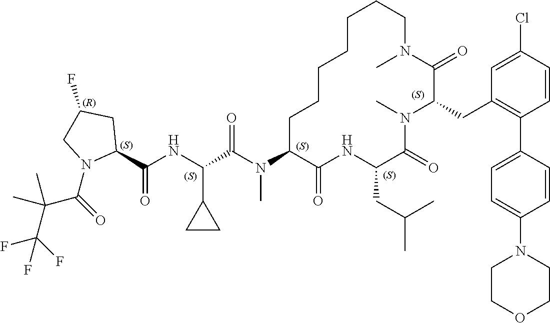 |
| 336 | 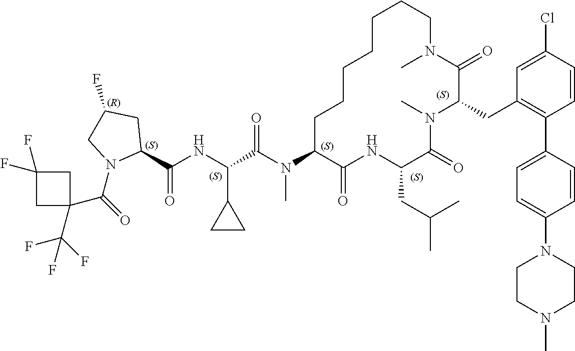 |
| 337 | 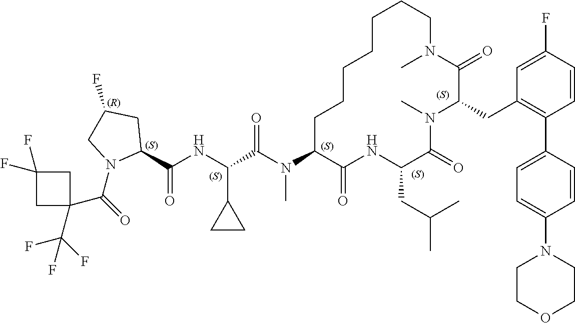 |
| 338 | 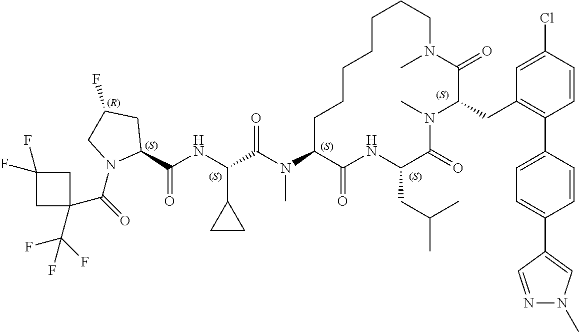 |
| 339 | 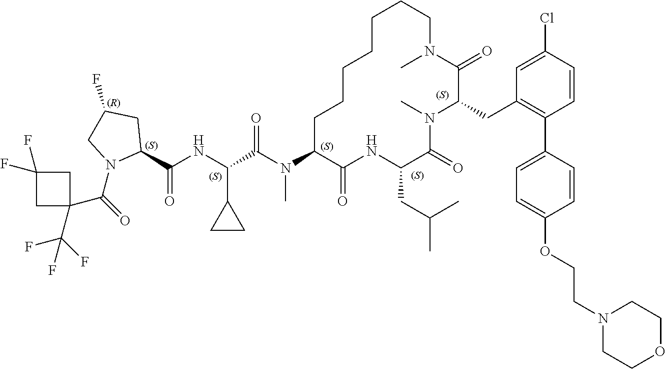 |
| 340 | 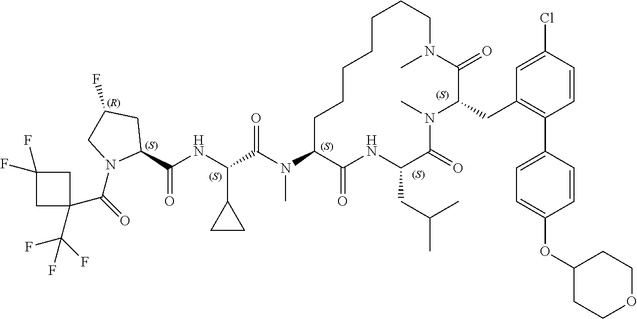 |
| 341 | 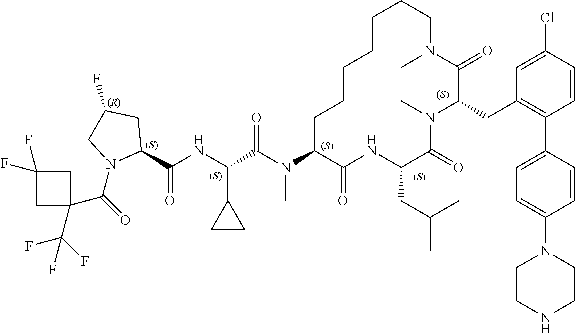 |
| 342 | 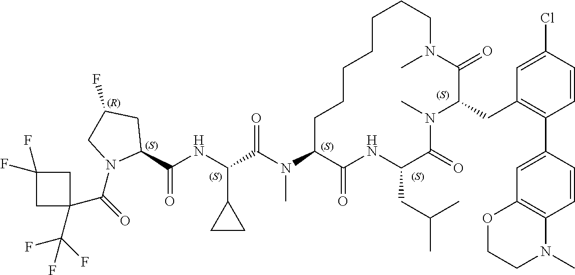 |
| 343 | 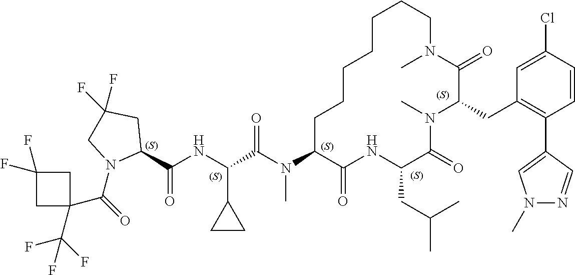 |
| 344 | 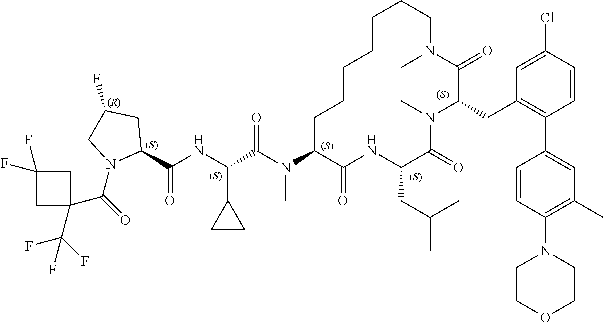 |
| 345 | 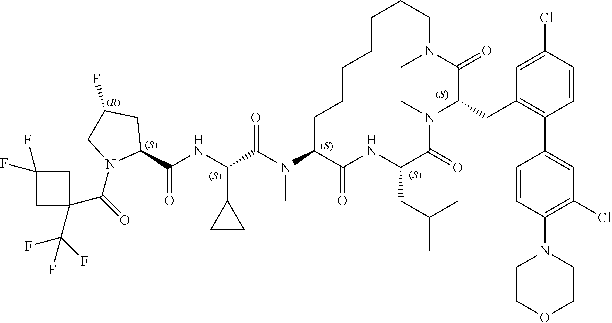 |
| 346 | 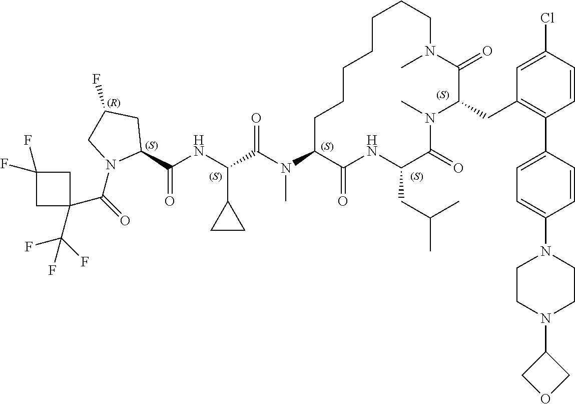 |
| 347 | 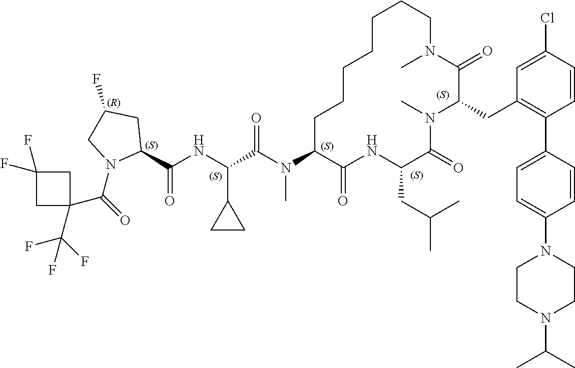 |
| 348 | 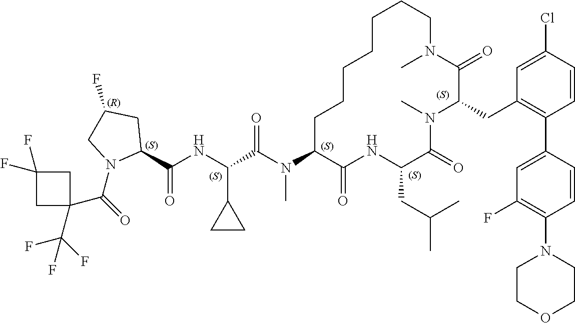 |
| 349 | 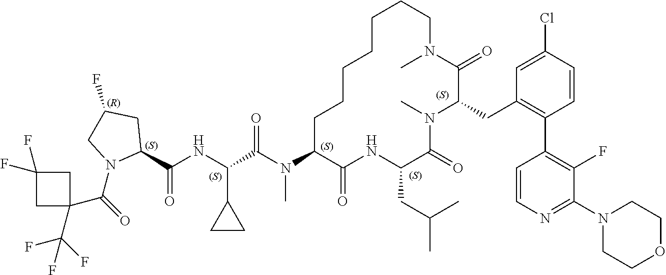 |
| 350 | 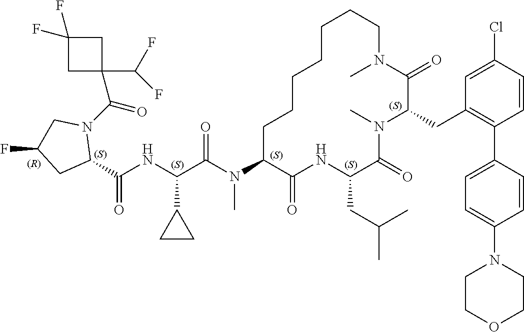 |
| 351 | 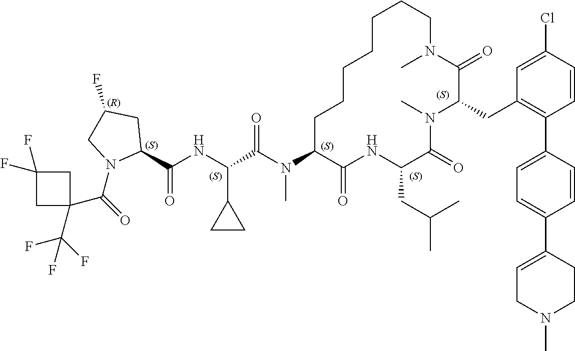 |
| 352 | 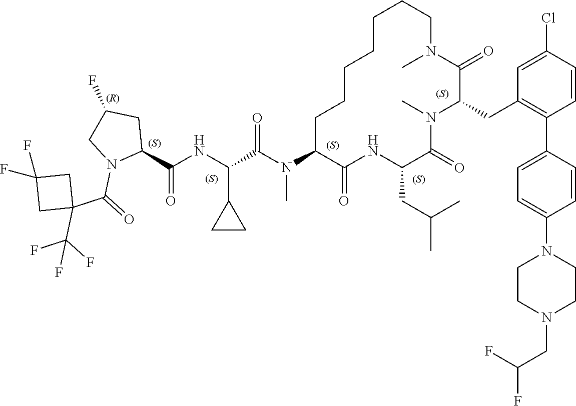 |
| 353 | 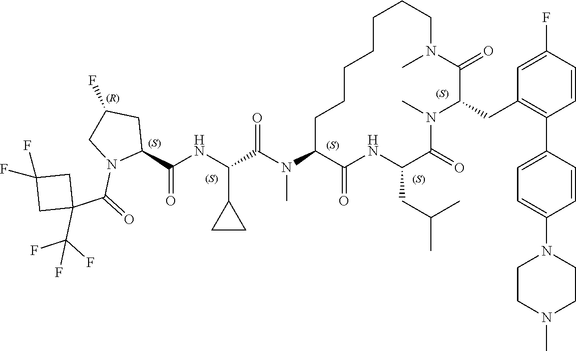 |
| 354 | 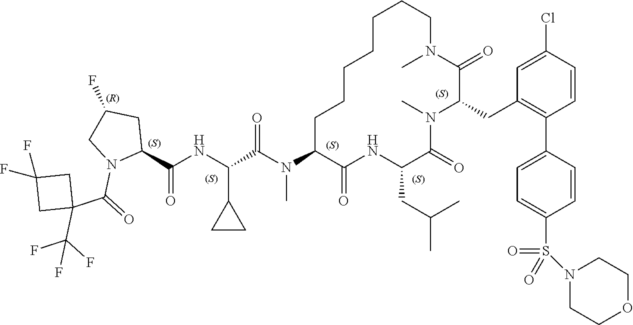 |
| 355 | 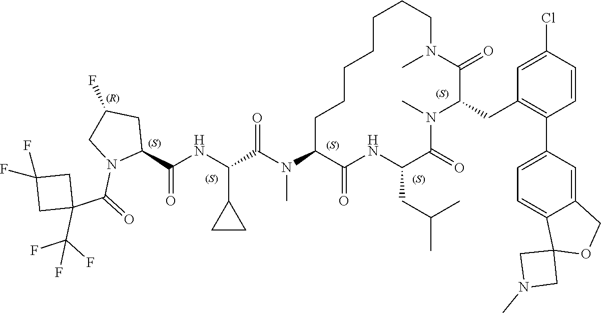 |
| 356 | 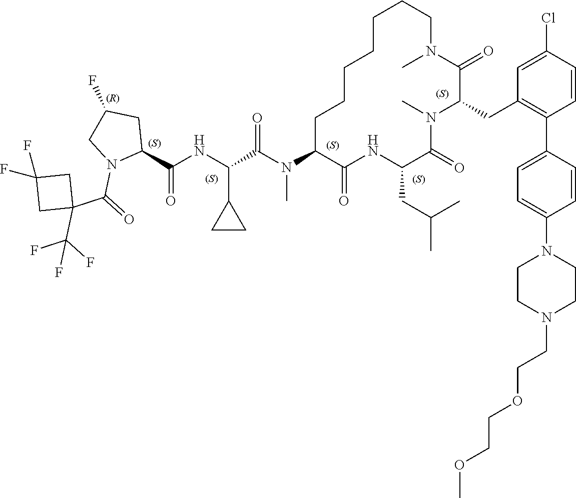 |
| 357 | 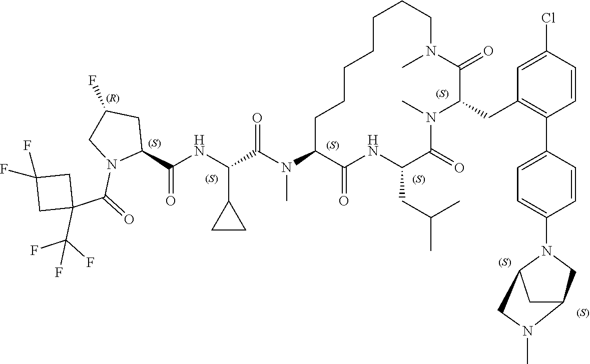 |
| 358 | 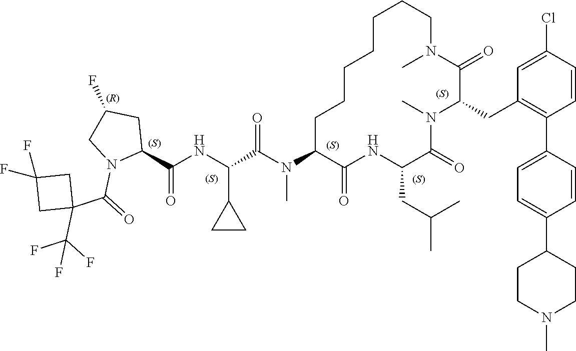 |
| 359 | 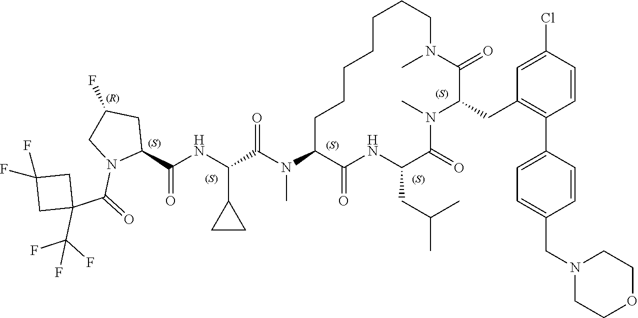 |
| 360 | 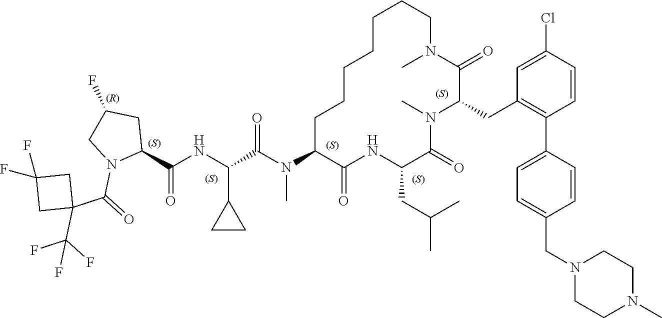 |
| 361 | 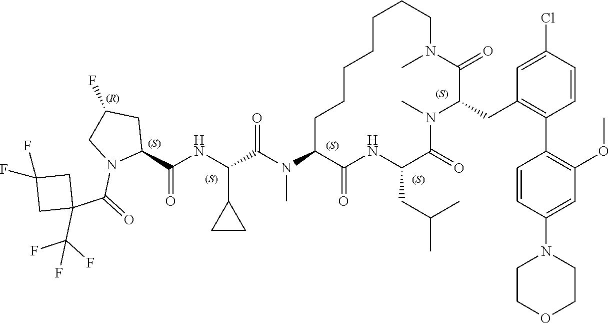 |
| 362 | 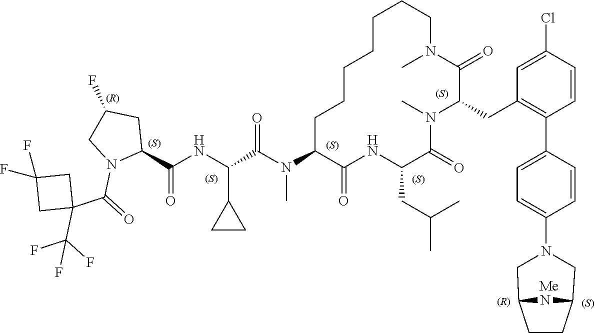 |
| 363 | 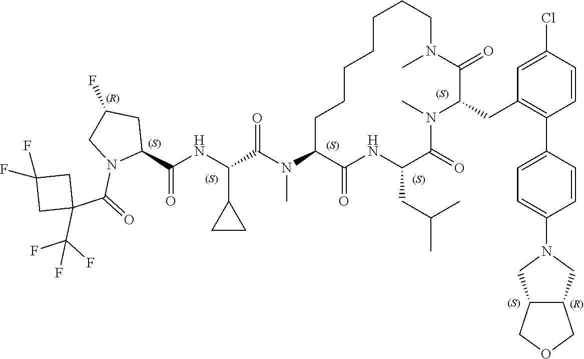 |
| 364 | 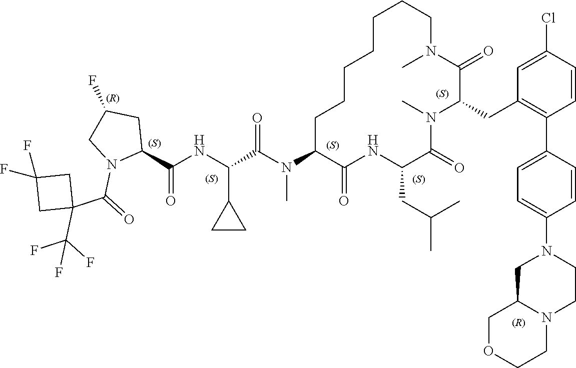 |
| 365 | 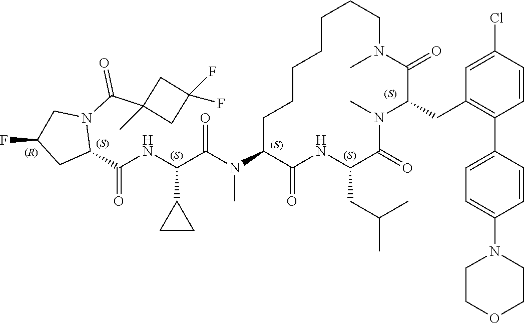 |
| 366 | 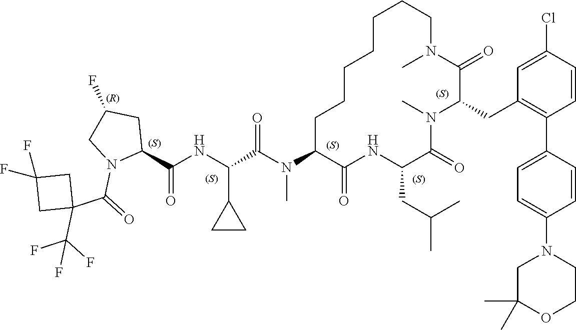 |
| 367 | 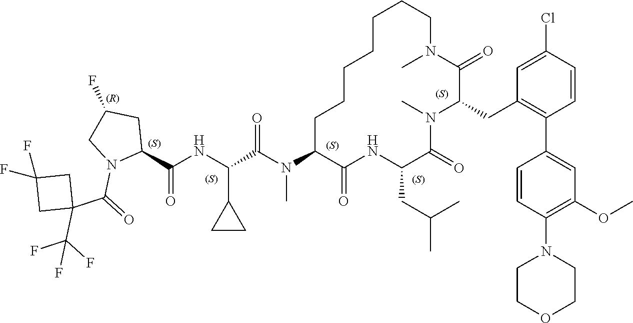 |
| 368 | 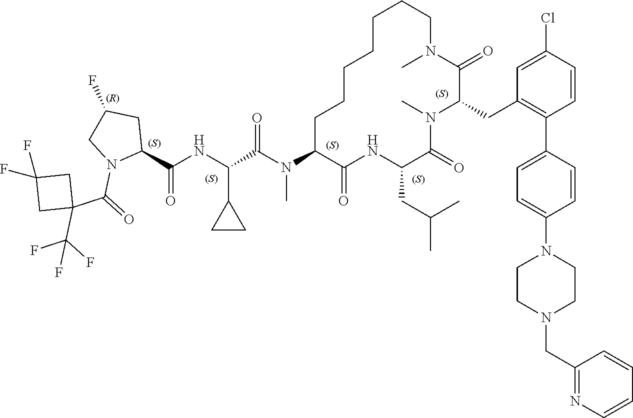 |
| 369 | 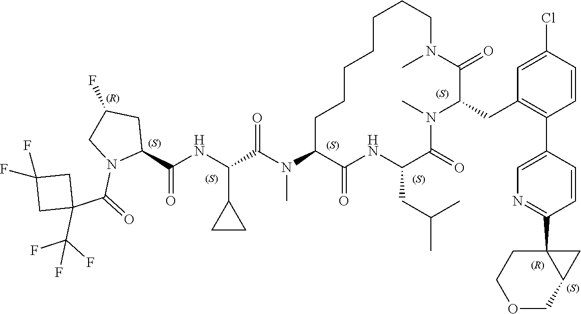 |
| 370 | 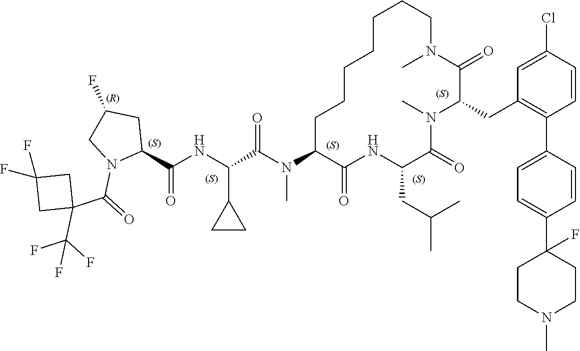 |
| 371 | 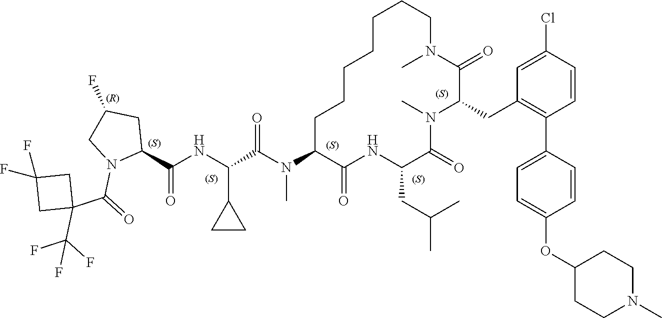 |
| 372 | 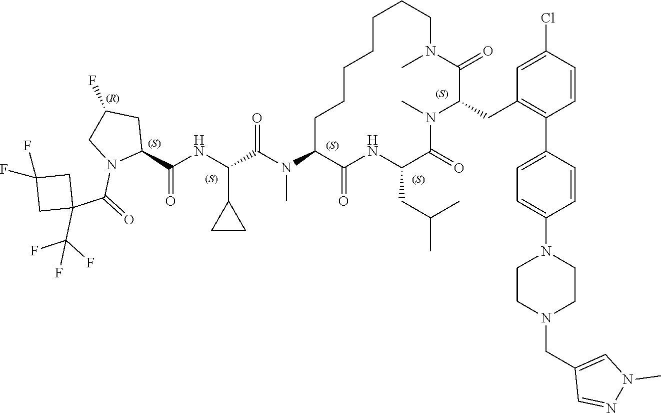 |
| 373 | 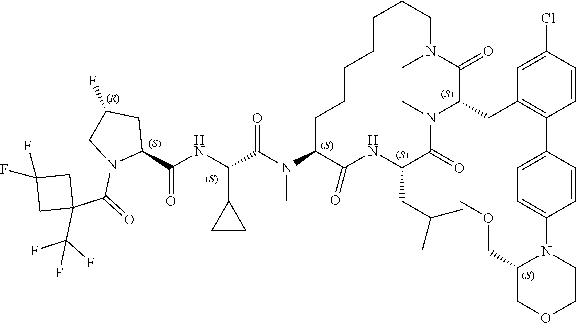 |
| 374 | 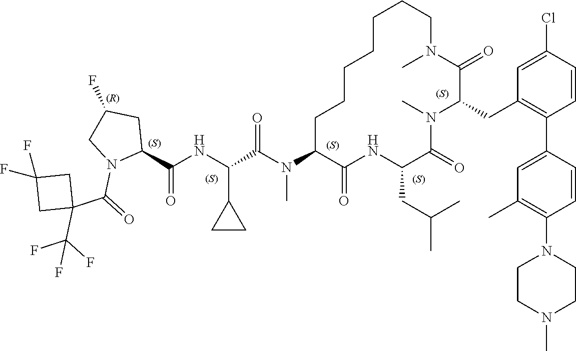 |
| 375 | 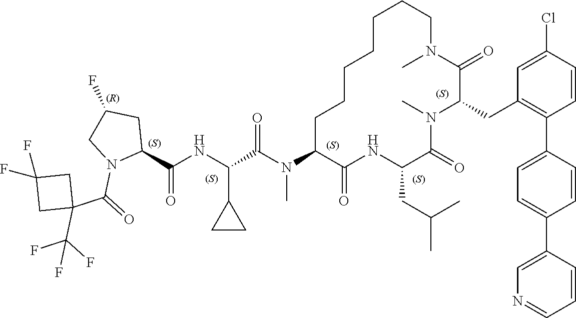 |
| 376 | 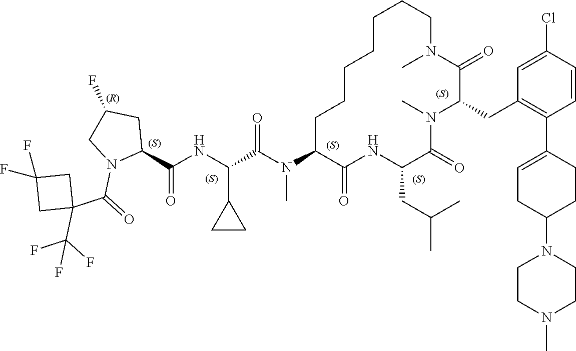 |
| 377 | 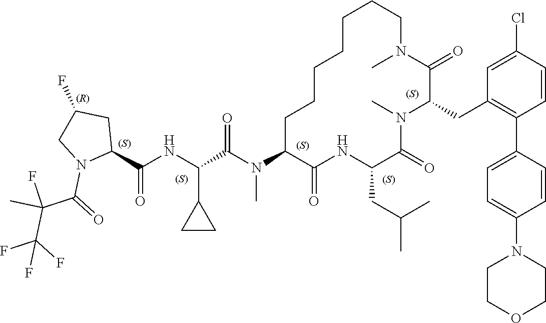 |
| 378 | 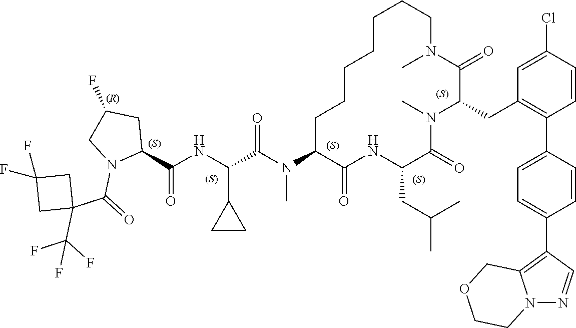 |
| 379 | 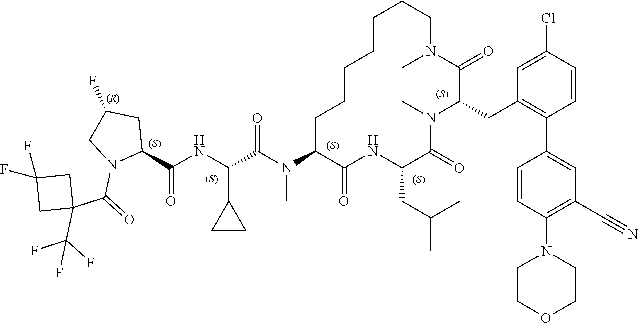 |
| 380 | 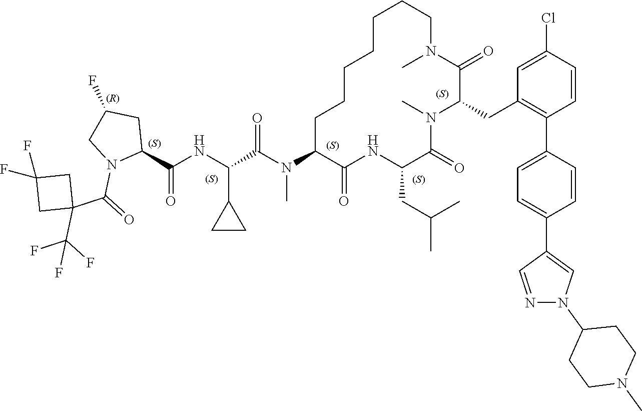 |
| 381 | 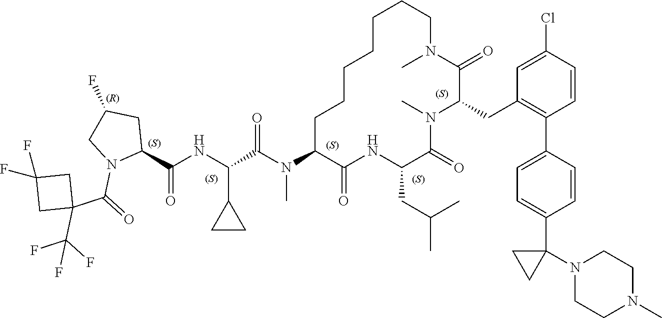 |
| 382 | 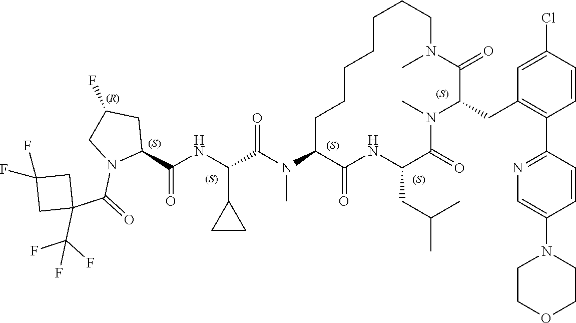 |
| 383 | 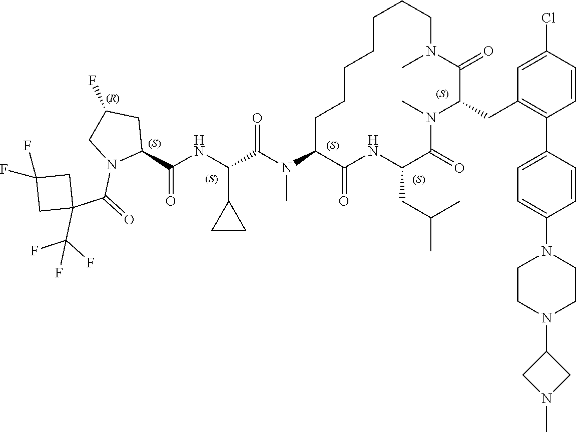 |
| 384 | 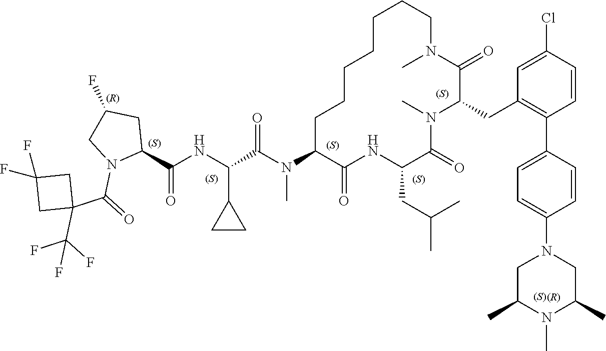 |
| 385 | 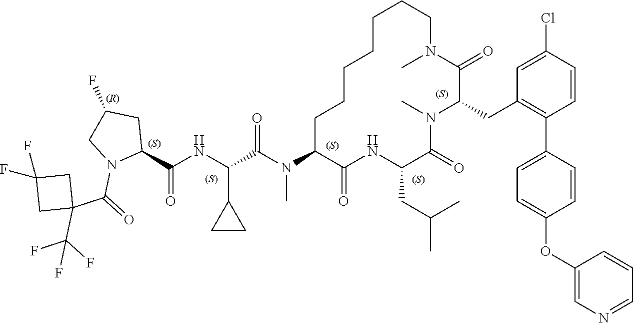 |
| 386 | 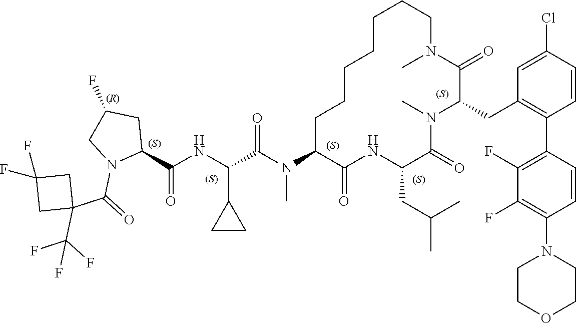 |
| 387 | 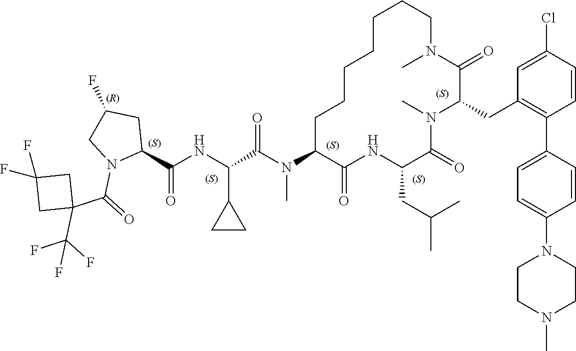 |
| 388 | 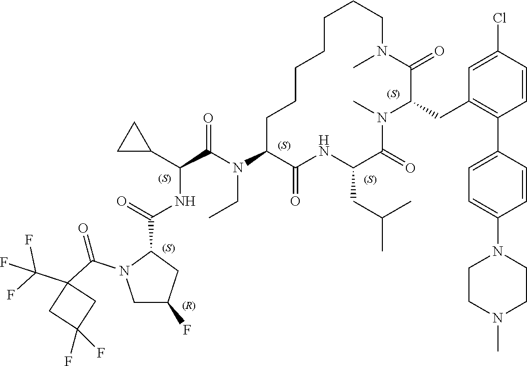 |
| 389 | 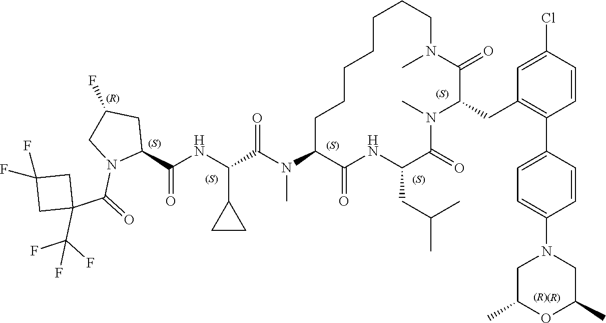 |
| 390 | 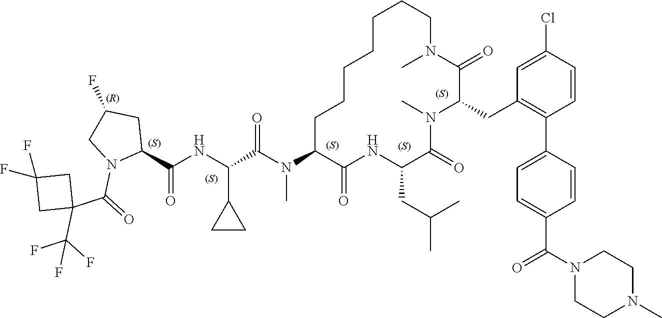 |
| 391 | 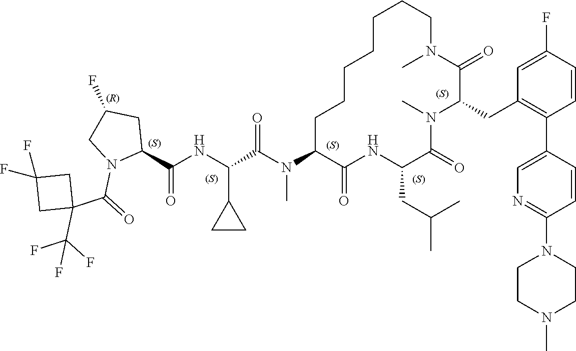 |
| 392 | 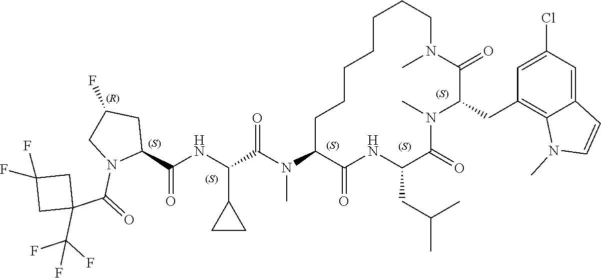 |
| 393 | 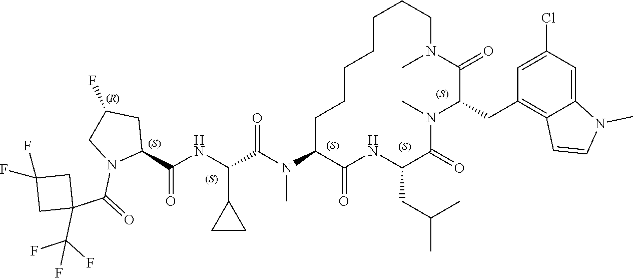 |
| 394 | 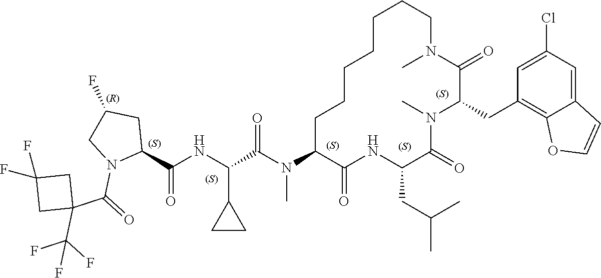 |
| 395 | 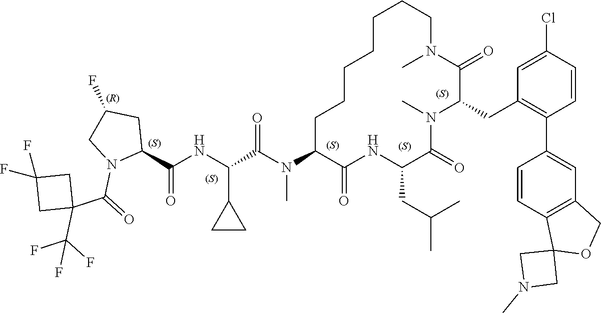 |
| 396 | 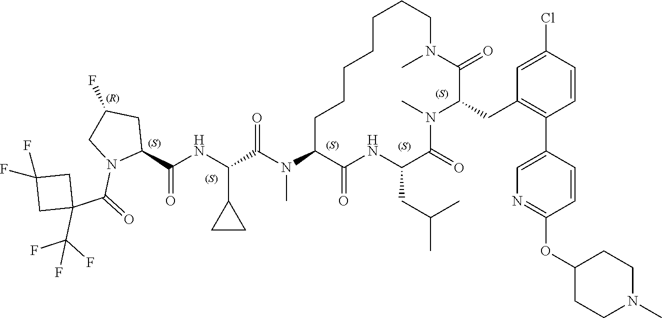 |
| 397 | 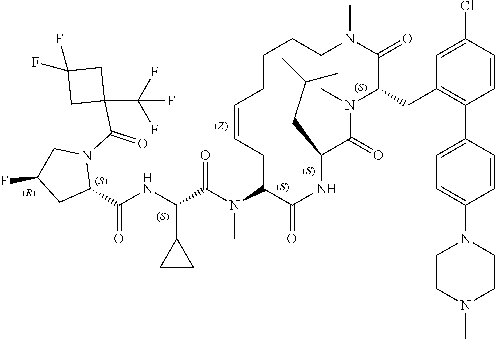 |
| 398 | 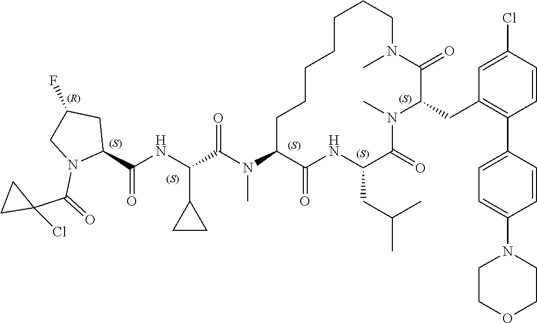 |
| 399 | 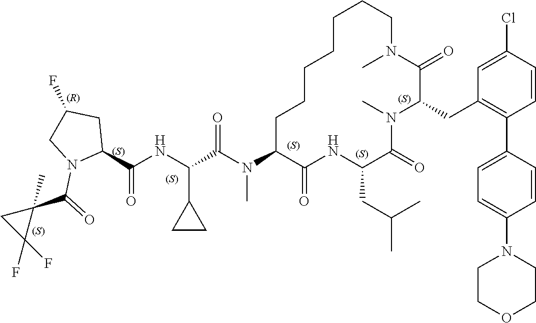 |
| 400 | 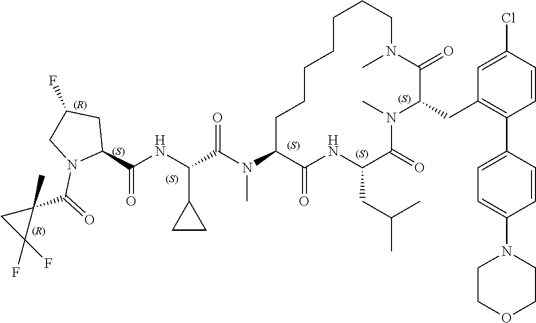 |
| 401 | 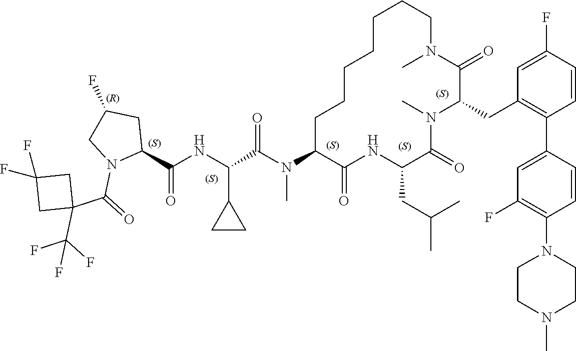 |
| 402 | 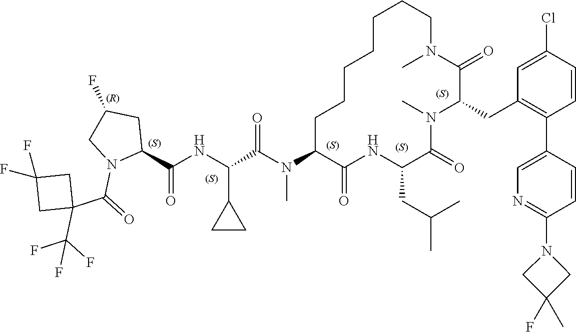 |
| 403 | 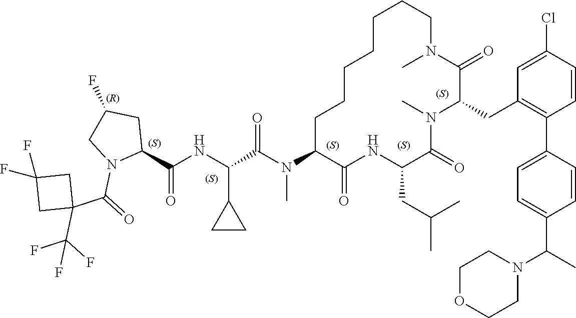 |
| 404 | 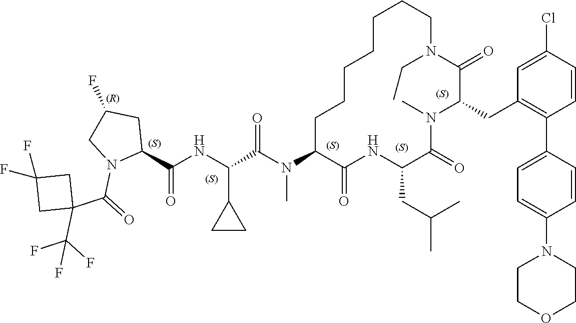 |
| 405 | 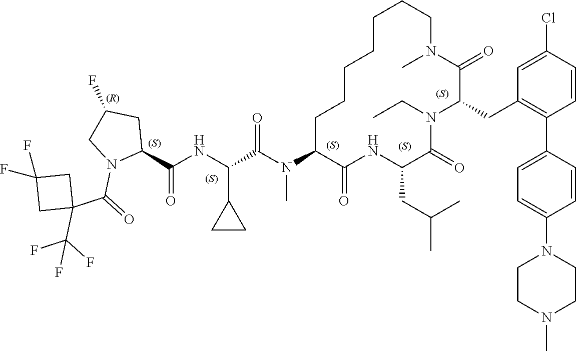 |
| 406 | 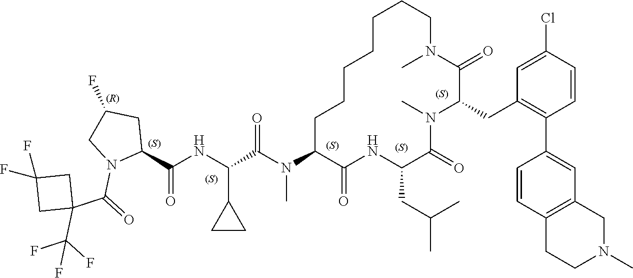 |
| 407 | 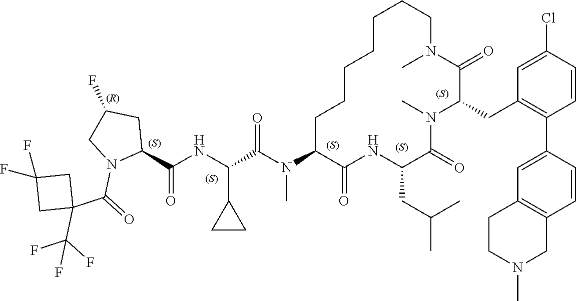 |
| 408 | 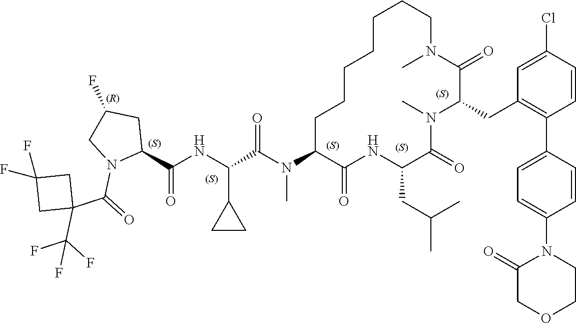 |
| 409 | 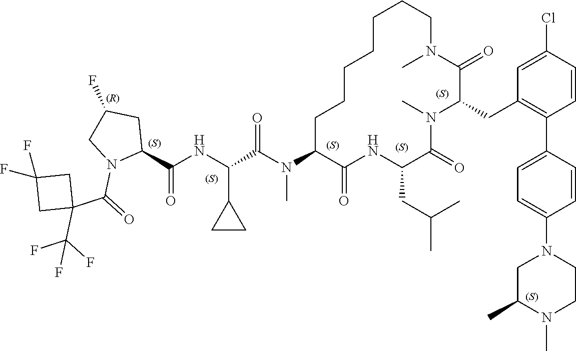 |
| 410 | 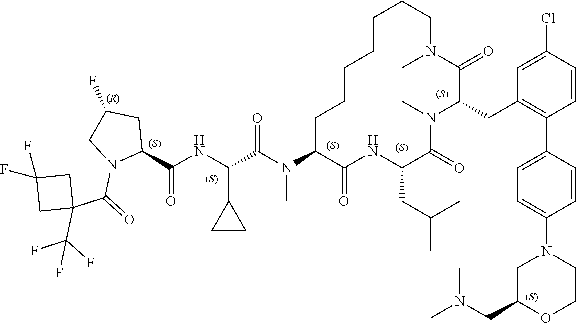 |
| 411 | 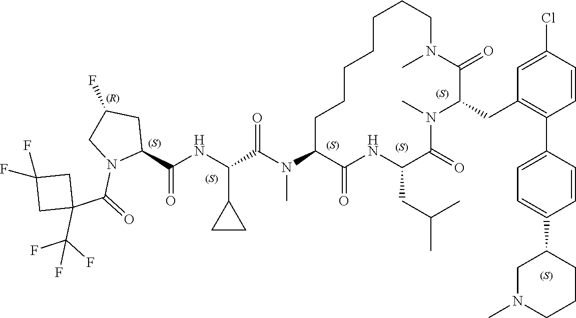 |
| 412 | 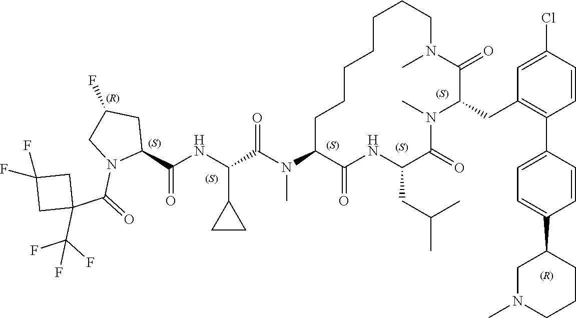 |
| 413 | 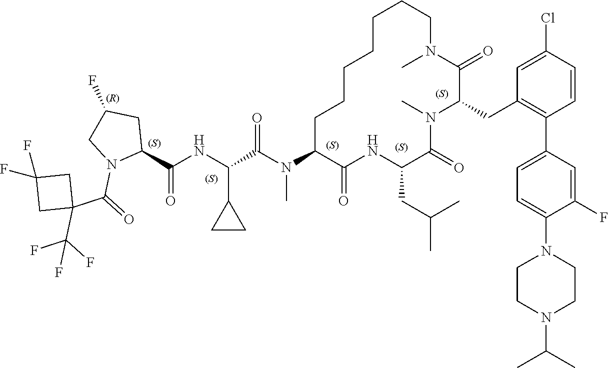 |
| 414 | 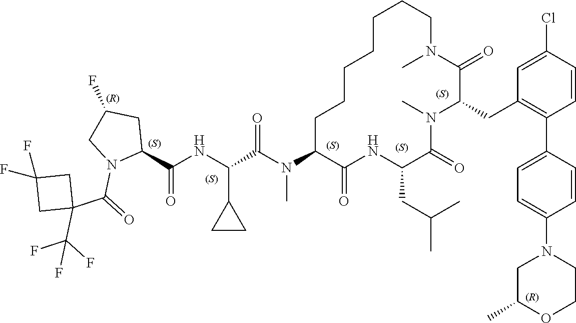 |
| 415 | 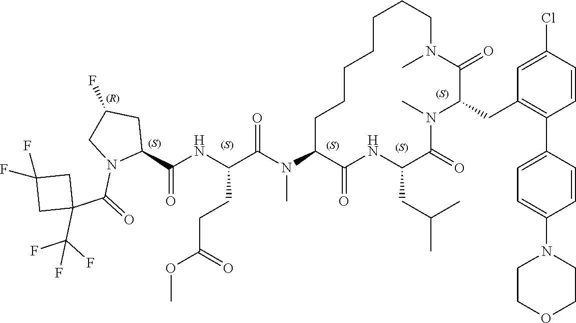 |
| 416 | 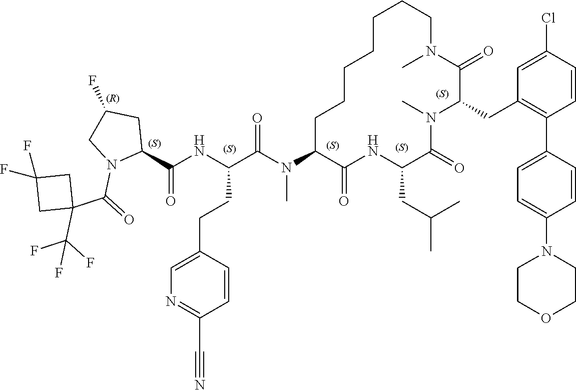 |
| 417 | 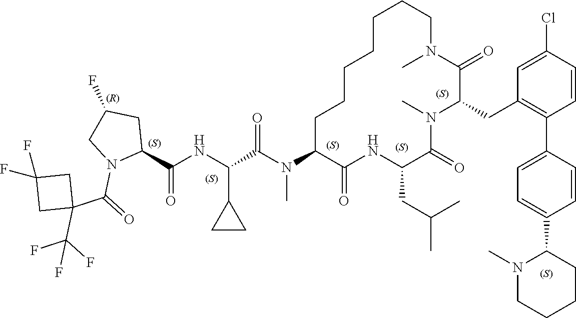 |
| 418 | 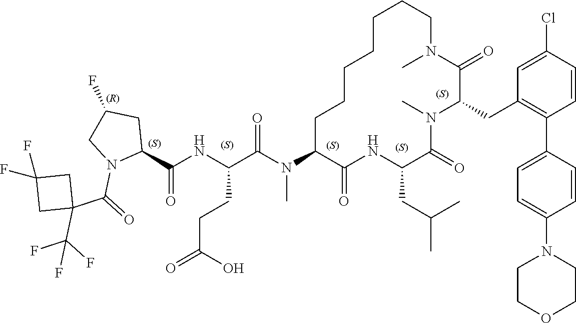 |
| 419 | 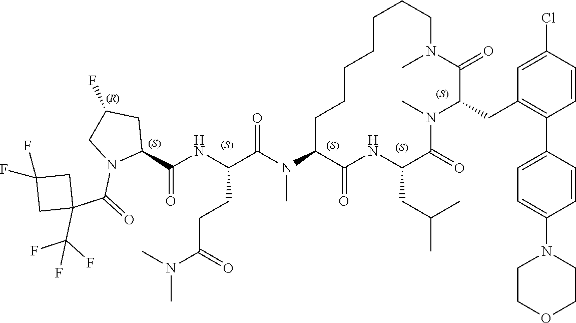 |
| 420 | 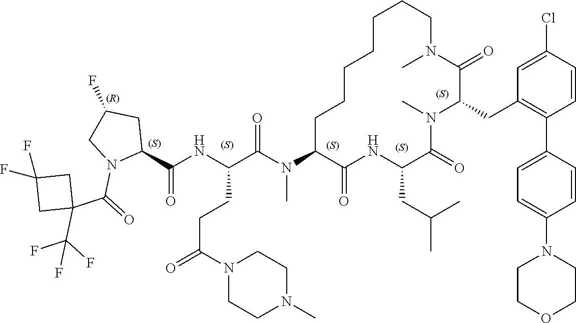 |
| 421 | 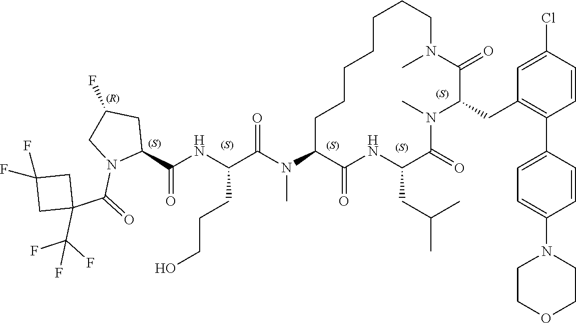 |
| 422 | 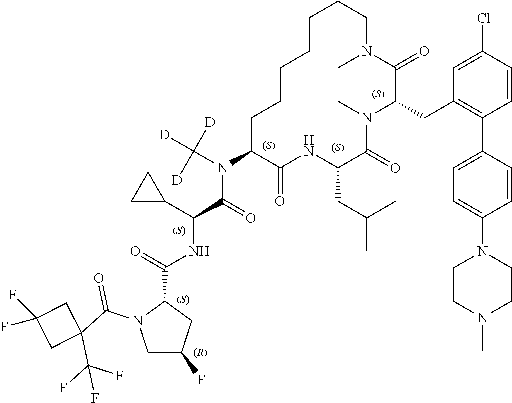 |
| 423 | 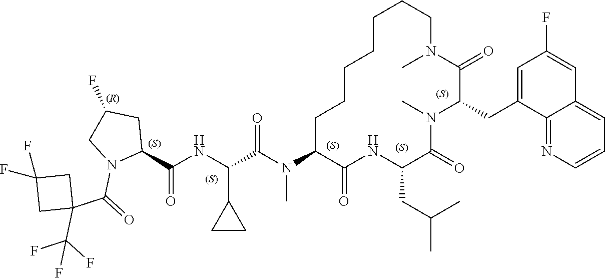 |
| 424 | 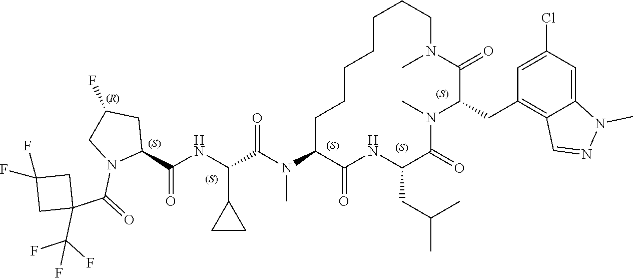 |
| 425 | 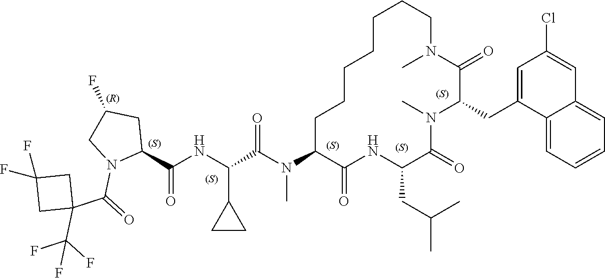 |
| 426 | 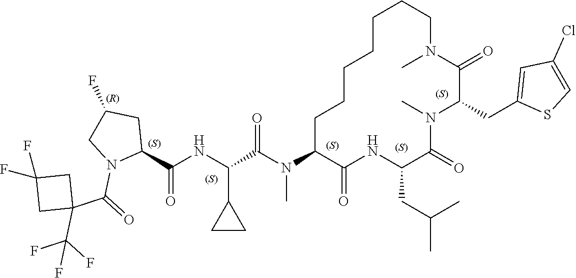 |

### B. Biological Examples

### 1. Fluorescence Polarization Assay

Binding affinity for the compounds of Formula (I) were determined by Fluorescence Polarization (FP) competitive assay based on previously established protocols (Andrews et. al., Org Biomol Chem., 2004. 2(19):2735-41; Premnath et. al., J Med Chem., 2015. 58(1):433-42.) with modifications as described below. Cyclin/CDK protein complexes were sourced as follows: CyclinA2/CDK2 (CRELUX Protein Services), CyclinB1/CDK1 (Eurofins, discovery. Cat. No. 14-450) and CyclinE1/CDK2 (Eurofins, discovery. Cat. No. 14-475).

FP binding assays were performed in 25 mM HEPES pH 7.5, 100 mM NaCl, 1 mM DTT, 0.01% NP-40 and 1 mg/mL BSA for all 3 protein complexes in black 96-well plates. After experimental plates are set, they were equilibrated by gentle mixing by placing them on an orbital shaker at 100 rpm for 2 hours at rt and then read on a SpectraMax i3X Multi-Mode Microplate Detection platform.

Affinity of the Cyclin/Cdk complexed for the fluorescent labeled probe was determined by adding increasing concentration of each protein complex in buffer containing a carboxyfluorescein labeled probe (FAM probe) at 2 nM (preparation of FAM probe is described below). The half maximal concentration of protein needed for the maximal FP signal were 2 nM for Cyclin A2/Cdk2, 9 nM for Cyclin B1/Cdk1 and 3 nM for Cyclin E1/Cdk2. Methods to prepare the FAM probe are described in the heading below.

The protein concentration used for the competitive FP assays were 8 nM for Cyclin A2/Cdk2 and 10 nM for Cyclin B1/Cdk1 and Cyclin E1/Cdk2 with 2 nM of FAM probe FAM probe. Under these conditions, the dynamic range was about 120 mP 100% binding of FAM probe and complete inhibition of binding by excess of an unlabeled competitor compound, with all experiment showing a Z′ factor >0.80. IC₅₀ for test compounds were determined in eight-point serial dilution dose response curves. Reported IC₅₀ are the average of 2-3 independent experiments. Data from these assays are reported in Table 5.

TABLE 5
Cyclin A, B, and E Activity Data of Exemplary Compounds

| | Cyclin A | Cyclin B | Cyclin E |
|---|---|---|---|
| Example | FP IC50 | FP IC50 | FP IC50 |
| Number | (μM) | (μM) | (μM) |
| 1 | 0.21 | 0.02 | 0.44 |
| 2 | 0.11 | 0.02 | 0.10 |
| 3 | 0.62 | 0.02 | 0.68 |
| 4 | 0.09 | 0.02 | 0.10 |
| 5-P1 | 0.79 | 0.02 | 0.70 |
| 5-P2 | 0.56 | 0.02 | 0.46 |
| 6-P1 | 0.16 | 0.02 | 0.50 |
| 6-P2 | 0.10 | 0.02 | 0.24 |
| 7 | 0.06 | 0.02 | 0.29 |
| 8 | 0.03 | 0.02 | 0.10 |
| 9 | 0.25 | 0.02 | 0.40 |
| 10 | 0.10 | 0.02 | 0.12 |
| 11 | 0.17 | 0.02 | 0.10 |
| 12 | 0.06 | 0.02 | 0.10 |
| 13 | 0.10 | 0.02 | 0.10 |
| 14 | 0.10 | 0.02 | 0.34 |
| 15 | 0.28 | 0.02 | 1.04 |
| 16-P1 | 0.74 | 0.02 | 3.15 |
| 16-P2 | 0.60 | 0.02 | 2.71 |
| 17 | 0.09 | 0.02 | 4.11 |
| 18 | 0.09 | 0.02 | 6.19 |
| 19 | 0.43 | 0.02 | 20.00 |
| 20 | 0.02 | 0.02 | 1.52 |
| 21 | 0.02 | 0.02 | 1.13 |
| 22 | 0.21 | 0.02 | 20.00 |
| 23 | 0.12 | 0.02 | 5.47 |
| 24 | 0.06 | 0.02 | 1.60 |
| 25 | 0.04 | 0.02 | 0.23 |
| 26 | 0.06 | 0.02 | 1.17 |
| 27 | 0.09 | 0.02 | 1.90 |
| 28 | 0.04 | 0.02 | 0.74 |
| 29 | 0.03 | 0.02 | 0.69 |
| 30 | 0.24 | 0.02 | 5.05 |
| 31 | 0.03 | 0.02 | 2.20 |
| 32 | 0.05 | 0.02 | 0.59 |
| 33 | 0.02 | 0.02 | 0.27 |
| 34 | 0.02 | 0.02 | 0.21 |
| 35 | 0.12 | 0.02 | 1.69 |
| 36 | 0.03 | 0.02 | 0.82 |
| 37 | 0.05 | 0.02 | 0.37 |
| 38 | 0.18 | 0.02 | 1.76 |
| 39 | 0.06 | 0.02 | 0.94 |
| 40 | 0.05 | 0.02 | 0.24 |
| 41 | 0.16 | 0.02 | 0.81 |
| 42 | 0.04 | 0.02 | 0.44 |
| 43 | 0.04 | 0.02 | 0.37 |
| 44 | 0.03 | 0.02 | 0.27 |
| 45 | 0.10 | 0.02 | 0.33 |
| 46 | 0.02 | 0.02 | 0.35 |
| 47 | 0.04 | 0.02 | 0.80 |
| 48 | 0.08 | 0.02 | 0.13 |
| 49 | 0.02 | 0.02 | 0.35 |
| 50 | 0.02 | 0.02 | 1.22 |
| 51 | 0.02 | 0.02 | 0.89 |
| 52 | 0.04 | 0.02 | 1.13 |
| 53 | 0.09 | 0.02 | 12.40 |
| 54 | 0.39 | 0.03 | 3.39 |
| 55 | 0.70 | 0.08 | 3.93 |
| 56-P1 | 0.86 | 0.04 | 20.00 |
| 56-P2 | 0.59 | 0.03 | 20.00 |
| 57-P1 | 0.02 | 0.02 | 1.41 |
| 57-P2 | 0.02 | 0.02 | 1.31 |
| 58 | 0.02 | 0.02 | 0.62 |
| 59-P1 | 0.12 | 0.02 | 0.85 |
| 59-P2 | 0.18 | 0.02 | 0.87 |
| 60 | 0.02 | 0.02 | 0.74 |
| 61 | 0.02 | 0.02 | 1.01 |
| 62 | 0.03 | 0.02 | 0.39 |
| 63 | 0.12 | 0.02 | 3.51 |
| 64 | 0.38 | 0.02 | 20.00 |
| 65 | 0.02 | 0.02 | 0.27 |
| 66 | 0.02 | 0.02 | 0.45 |
| 67 | 0.02 | 0.02 | 0.50 |
| 68 | 0.02 | 0.02 | 0.12 |
| 69 | 0.02 | 0.02 | 0.11 |
| 70 | 0.02 | 0.02 | 0.29 |
| 71 | 0.02 | 0.02 | 1.59 |
| 72 | 0.03 | 0.02 | 1.05 |
| 73 | 0.03 | 0.02 | 0.93 |
| 74 | 0.05 | 0.02 | 3.62 |
| 75 | 0.02 | 0.02 | 0.44 |
| 76 | 0.02 | 0.02 | 0.88 |
| 77 | 0.02 | 0.02 | 1.71 |
| 78 | 0.04 | 0.02 | 0.64 |
| 79 | 0.05 | 0.02 | 0.63 |
| 80 | 0.04 | 0.02 | 0.30 |
| 81 | 0.04 | 0.02 | 0.30 |
| 82 | 0.02 | 0.02 | 0.22 |
| 83 | 0.04 | 0.02 | 0.10 |
| 84 | 0.05 | 0.02 | 0.29 |
| 85 | 0.06 | 0.02 | 0.28 |
| 86 | 0.06 | 0.02 | 0.25 |
| 87 | 0.18 | 0.02 | 0.89 |
| 88 | 0.05 | 0.02 | 0.20 |
| 89 | 0.12 | 0.02 | 5.41 |
| 90 | 0.09 | 0.02 | 3.70 |
| 91 | 0.10 | 0.02 | 5.73 |
| 92 | 0.02 | 0.02 | 0.10 |
| 93 | 0.02 | 0.02 | 0.14 |
| 94 | 0.02 | 0.02 | 0.18 |
| 95 | 0.05 | 0.02 | 0.53 |
| 96 | 0.02 | 0.02 | 0.12 |
| 97 | 0.02 | 0.02 | 1.45 |
| 98 | 0.02 | 0.02 | 1.10 |
| 99 | 0.02 | 0.02 | 1.54 |
| 100 | 0.02 | 0.02 | 0.45 |
| 101 | 0.03 | 0.02 | 0.47 |
| 102-P1 | 0.31 | 0.02 | 1.11 |
| 102-P2 | 0.12 | 0.02 | 0.31 |
| 103 | 0.06 | 0.02 | 0.35 |
| 104 | 0.16 | 0.02 | 0.49 |
| 105 | 0.05 | 0.02 | 0.29 |
| 106 | 0.12 | 0.02 | 0.24 |
| 107-P1 | 0.25 | 0.02 | 0.54 |
| 107-P2 | 0.11 | 0.02 | 0.18 |
| 108 | 0.05 | 0.02 | 0.18 |
| 109 | 0.04 | 0.02 | 0.17 |
| 110 | 0.02 | 0.02 | 0.33 |
| 111 | 0.02 | 0.02 | 0.28 |
| 112 | 0.02 | 0.02 | 0.34 |
| 113 | 0.02 | 0.02 | 0.31 |
| 114 | 0.02 | 0.02 | 0.40 |
| 115 | 0.04 | 0.02 | 0.17 |
| 116 | 0.04 | 0.02 | 0.33 |
| 117-P1 | 0.08 | 0.02 | 0.34 |
| 117-P2 | 0.06 | 0.02 | 0.16 |
| 118-P1 | 0.11 | 0.02 | 0.74 |
| 118-P2 | 0.06 | 0.02 | 0.30 |
| 119 | 0.03 | 0.02 | 0.10 |
| 120 | 0.03 | 0.02 | 0.21 |
| 121 | 0.05 | 0.02 | 0.13 |
| 122 | 0.02 | 0.02 | 0.10 |
| 123 | 0.03 | 0.02 | 0.12 |
| 124 | 0.02 | 0.02 | 0.54 |
| 125 | 1.01 | 0.02 | 1.14 |
| 126 | 0.50 | 0.02 | 0.62 |
| 127 | 0.08 | 0.02 | 0.23 |
| 128 | 0.08 | 0.02 | 0.19 |
| 129 | 0.21 | 0.02 | 0.82 |
| 130 | 0.07 | 0.02 | 0.31 |
| 131 | 0.03 | 0.02 | 0.24 |
| 132 | 0.71 | 0.02 | 0.45 |
| 133 | 0.47 | 0.02 | 0.34 |
| 134 | 0.10 | 0.02 | 0.12 |
| 135 | 0.08 | 0.02 | 0.12 |
| 136-P1 | 0.14 | 0.02 | 0.32 |
| 136-P2 | 0.08 | 0.02 | 0.12 |
| 137 | 0.06 | 0.02 | 0.13 |
| 138 | 0.03 | 0.02 | 0.11 |
| 139 | 0.02 | 0.02 | 0.10 |
| 140 | 0.02 | 0.02 | 0.12 |
| 141 | 0.04 | 0.02 | 0.10 |
| 142 | 0.04 | 0.02 | 0.15 |
| 143 | 0.02 | 0.02 | 0.94 |
| 144 | 0.02 | 0.02 | 0.84 |
| 145 | 0.02 | 0.02 | 1.30 |
| 146 | 0.03 | 0.02 | 0.10 |
| 147 | 0.02 | 0.02 | 0.10 |
| 148 | 0.03 | 0.02 | 0.31 |
| 149 | 0.02 | 0.02 | 0.56 |
| 150 | 0.02 | 0.02 | 0.21 |
| 151 | 0.02 | 0.02 | 0.22 |
| 152 | 1.02 | 0.02 | 1.90 |
| 153 | 0.47 | 0.02 | 1.00 |
| 154 | 0.15 | 0.02 | 0.48 |
| 155 | 0.10 | 0.02 | 0.41 |
| 156-P1 | 0.89 | 0.02 | 0.83 |
| 156-P2 | 0.35 | 0.02 | 0.24 |
| 157 | 0.36 | 0.02 | 0.41 |
| 158 | 0.10 | 0.02 | 0.21 |
| 159 | 0.08 | 0.02 | 0.20 |
| 160 | 0.02 | 0.02 | 0.27 |
| 161 | 0.02 | 0.02 | 0.12 |
| 162 | 0.02 | 0.02 | 0.22 |
| 163 | 0.02 | 0.02 | 0.10 |
| 164 | 0.07 | 0.02 | 0.93 |
| 165 | 0.11 | 0.02 | 5.47 |
| 166 | 0.06 | 0.02 | 2.30 |
| 167 | 0.02 | 0.02 | 0.28 |
| 168 | 0.02 | 0.02 | 0.16 |
| 169 | 0.03 | 0.02 | 1.09 |
| 170 | 0.05 | 0.02 | 2.22 |
| 171 | 0.08 | 0.02 | 4.21 |
| 172 | 0.06 | 0.02 | 2.43 |
| 173 | 0.02 | 0.02 | 0.10 |
| 174 | 0.02 | 0.02 | 0.10 |
| 175 | 0.02 | 0.02 | 0.13 |
| 176 | 0.02 | 0.02 | 0.10 |
| 177 | 0.02 | 0.02 | 0.46 |
| 178 | 0.05 | 0.02 | 5.46 |
| 179 | 0.03 | 0.02 | 1.13 |
| 180 | 0.03 | 0.02 | 1.86 |
| 181 | 0.04 | 0.02 | 1.58 |
| 182 | 0.02 | 0.02 | 1.17 |
| 183 | 0.03 | 0.02 | 0.28 |
| 184 | 0.02 | 0.02 | 0.10 |
| 185 | 0.02 | 0.02 | 0.11 |
| 186 | 0.02 | 0.02 | 0.10 |
| 187 | 0.02 | 0.02 | 0.34 |
| 188 | 0.02 | 0.02 | 0.23 |
| 189 | 0.05 | 0.02 | 0.85 |
| 190 | 0.02 | 0.02 | 0.66 |
| 191 | 0.02 | 0.02 | 0.26 |
| 192 | 0.02 | 0.02 | 1.14 |
| 193 | 0.02 | 0.02 | 0.71 |
| 194 | 0.02 | 0.02 | 0.54 |
| 195 | 0.04 | 0.02 | 0.18 |
| 196 | 0.03 | 0.02 | 0.74 |
| 197 | 0.02 | 0.02 | 0.31 |
| 198 | 0.05 | 0.02 | 1.45 |
| 199 | 0.03 | 0.02 | 0.91 |
| 200 | 0.03 | 0.02 | 0.89 |
| 201 | 0.03 | 0.02 | 1.95 |
| 202 | 0.05 | 0.02 | 1.49 |
| 203 | 0.04 | 0.02 | 1.26 |
| 204 | 0.02 | 0.02 | 0.16 |
| 205 | 0.03 | 0.02 | 0.48 |
| 206 | 0.02 | 0.02 | 0.20 |
| 207 | 0.05 | 0.02 | 2.73 |
| 208 | 0.21 | 0.02 | 6.40 |
| 209 | 0.02 | 0.02 | 0.54 |
| 210 | 0.42 | 0.02 | 20.00 |
| 211 | 0.05 | 0.02 | 1.11 |
| 212 | 0.07 | 0.02 | 4.16 |
| 213 | 0.03 | 0.02 | 0.19 |
| 214 | 0.05 | 0.02 | 0.50 |
| 215 | 0.02 | 0.02 | 0.20 |
| 216 | 0.03 | 0.02 | 0.47 |
| 217 | 0.03 | 0.02 | 0.76 |
| 218 | 0.10 | 0.02 | 2.22 |
| 219 | 0.10 | 0.02 | 1.43 |
| 220 | 0.09 | 0.02 | 0.68 |
| 221 | 0.06 | 0.02 | 0.18 |
| 222 | 0.04 | 0.02 | 0.23 |
| 223 | 0.02 | 0.02 | 0.10 |
| 224 | 0.03 | 0.02 | 0.10 |
| 225 | 0.03 | 0.02 | 0.19 |
| 226 | 0.02 | 0.02 | 0.10 |
| 227 | 0.03 | 0.02 | 0.12 |
| 228 | 0.03 | 0.02 | 0.25 |
| 229 | 0.02 | 0.02 | 0.10 |
| 230 | 0.03 | 0.02 | 0.14 |
| 231 | 0.06 | 0.02 | 2.94 |
| 232 | 0.05 | 0.02 | 2.87 |
| 233 | 0.05 | 0.02 | 1.60 |
| 234 | 0.03 | 0.02 | 0.83 |
| 235 | 0.14 | 0.02 | 6.08 |
| 236 | 0.03 | 0.02 | 0.78 |
| 237 | 0.02 | 0.02 | 1.14 |
| 238 | 0.03 | 0.02 | 0.99 |
| 239 | 0.02 | 0.02 | 0.42 |
| 240 | 0.02 | 0.02 | 0.14 |
| 241 | 0.02 | 0.02 | 0.20 |
| 242 | 0.03 | 0.02 | 0.70 |
| 243 | 0.05 | 0.02 | 0.69 |
| 244 | 0.06 | 0.02 | 0.58 |
| 245 | 0.04 | 0.02 | 0.22 |
| 246 | 0.02 | 0.02 | 0.64 |
| 247 | 0.02 | 0.02 | 0.31 |
| 248 | 0.02 | 0.02 | 0.10 |
| 249 | 0.02 | 0.02 | 0.59 |
| 250 | 0.03 | 0.02 | 1.62 |
| 251 | 0.04 | 0.02 | 0.93 |
| 252 | 0.03 | 0.02 | 0.38 |
| 253 | 0.02 | 0.02 | 0.33 |
| 254 | 0.03 | 0.02 | 0.97 |
| 255 | 0.02 | 0.02 | 0.21 |
| 256 | 0.03 | 0.02 | 0.48 |
| 257 | 0.02 | 0.02 | 0.12 |
| 258 | 0.07 | 0.02 | 0.12 |
| 259 | 0.07 | 0.02 | 0.28 |
| 260 | 0.05 | 0.02 | 0.10 |
| 261 | 0.02 | 0.02 | 0.43 |
| 262 | 0.02 | 0.02 | 0.91 |
| 263 | 0.03 | 0.02 | 0.20 |
| 264 | 0.09 | 0.02 | 0.29 |
| 265 | 0.02 | 0.02 | 0.64 |
| 266 | 0.02 | 0.02 | 2.22 |
| 267 | 0.02 | 0.02 | 1.95 |
| 268 | 0.06 | 0.02 | 0.10 |
| 269 | 0.06 | 0.02 | 0.10 |
| 270 | 0.06 | 0.02 | 0.37 |
| 271 | 0.03 | 0.02 | 0.24 |
| 272 | 0.05 | 0.02 | 1.60 |
| 273 | 0.05 | 0.02 | 1.94 |
| 274 | 0.03 | 0.02 | 0.10 |
| 275 | 0.05 | 0.02 | 0.23 |
| 276 | 0.03 | 0.02 | 0.70 |
| 277 | 0.60 | 0.02 | 20.00 |
| 278 | 0.07 | 0.02 | 0.28 |
| 279 | 0.10 | 0.02 | 1.92 |
| 280 | 0.04 | 0.02 | 0.32 |
| 281 | 0.04 | 0.02 | 0.53 |
| 282 | 0.03 | 0.02 | 0.10 |
| 283 | 0.03 | 0.02 | 0.18 |
| 284 | 0.02 | 0.02 | 1.74 |
| 285 | 0.06 | 0.02 | 1.70 |
| 286 | 0.08 | 0.02 | 4.02 |
| 287 | 0.06 | 0.02 | 1.94 |
| 288 | 0.03 | 0.02 | 0.36 |
| 289 | 0.03 | 0.02 | 0.32 |
| 290 | 0.12 | 0.02 | 1.79 |
| 291 | 0.07 | 0.02 | 5.16 |
| 292 | 0.06 | 0.02 | 2.88 |
| 293 | 0.10 | 0.02 | 1.99 |
| 294 | 0.08 | 0.02 | 1.44 |
| 295 | 0.07 | 0.02 | 3.69 |
| 296 | 0.07 | 0.02 | 2.81 |
| 297 | 0.04 | 0.02 | 1.27 |
| 298 | 0.05 | 0.02 | 0.44 |
| 299 | 0.02 | 0.02 | 0.13 |
| 300 | 0.03 | 0.02 | 0.68 |
| 301 | 0.04 | 0.02 | 0.32 |
| 302 | 0.05 | 0.02 | 3.56 |
| 303 | 0.03 | 0.02 | 0.10 |
| 304 | 0.04 | 0.02 | 0.19 |
| 305 | 0.05 | 0.02 | 1.75 |
| 306 | 0.02 | 0.02 | 0.20 |
| 307 | 0.02 | 0.02 | 0.44 |
| 308 | 0.02 | 0.02 | 0.84 |
| 309 | 0.02 | 0.02 | 0.93 |
| 310 | 0.08 | 0.02 | 0.99 |
| 311 | 0.05 | 0.02 | 0.20 |
| 312 | 0.02 | 0.02 | 0.74 |
| 313 | 0.02 | 0.02 | 0.64 |
| 314 | 0.03 | 0.02 | 0.14 |
| 315 | 0.03 | 0.02 | 0.42 |
| 316 | 0.02 | 0.02 | 0.29 |
| 317 | 0.03 | 0.02 | 0.25 |
| 318 | 0.02 | 0.02 | 0.28 |
| 319 | 0.03 | 0.02 | 1.35 |
| 320 | 0.03 | 0.02 | 1.45 |
| 321 | 0.02 | 0.02 | 0.10 |
| 322 | 0.08 | 0.02 | 0.53 |
| 323 | 0.03 | 0.02 | 0.24 |
| 324 | 0.06 | 0.02 | 0.52 |
| 325 | 0.02 | 0.02 | 0.30 |
| 326 | 0.02 | 0.02 | 0.15 |
| 327 | 0.02 | 0.02 | 0.40 |
| 328 | 0.02 | 0.02 | 0.21 |
| 329 | 0.02 | 0.02 | 0.25 |
| 330 | 0.03 | 0.02 | 0.10 |
| 331 | 0.17 | 0.02 | 1.14 |
| 332 | 0.02 | 0.02 | 0.10 |
| 333 | 0.02 | 0.02 | 1.15 |
| 334 | 0.02 | 0.02 | 0.19 |
| 335 | 0.02 | 0.02 | 0.16 |
| 336 | 0.02 | 0.02 | 0.42 |
| 337 | 0.02 | 0.02 | 0.19 |
| 338 | 0.03 | 0.02 | 1.70 |
| 339 | 0.02 | 0.02 | 0.40 |
| 340 | 0.02 | 0.02 | 1.65 |
| 341 | 0.02 | 0.02 | 0.51 |
| 342 | 0.02 | 0.02 | 0.59 |
| 343 | 0.02 | 0.02 | 0.67 |
| 344 | 0.02 | 0.02 | 1.27 |
| 345 | 0.03 | 0.02 | 3.05 |
| 346 | 0.02 | 0.02 | 0.36 |
| 347 | 0.02 | 0.02 | 0.65 |
| 348 | 0.02 | 0.02 | 0.88 |
| 349 | 0.02 | 0.02 | 0.23 |
| 350 | 0.02 | 0.02 | 0.88 |
| 351 | 0.02 | 0.02 | 0.97 |
| 352 | 0.02 | 0.02 | 0.51 |
| 353 | 0.02 | 0.02 | 0.22 |
| 354 | 0.03 | 0.02 | 1.34 |
| 355 | 0.02 | 0.02 | 1.76 |
| 356 | 0.02 | 0.02 | 0.34 |
| 357 | 0.03 | 0.02 | 1.97 |
| 358 | 0.02 | 0.02 | 0.55 |
| 359 | 0.02 | 0.02 | 0.68 |
| 360 | 0.02 | 0.02 | 0.86 |
| 361 | 0.05 | 0.02 | 1.84 |
| 362 | 0.02 | 0.02 | 0.60 |
| 363 | 0.02 | 0.02 | 0.47 |
| 364 | 0.02 | 0.02 | 0.43 |
| 365 | 0.02 | 0.02 | 1.17 |
| 366 | 0.02 | 0.02 | 1.05 |
| 367 | 0.02 | 0.02 | 1.10 |
| 368 | 0.02 | 0.02 | 0.48 |
| 369 | 0.02 | 0.02 | 1.49 |
| 370 | 0.02 | 0.02 | 0.78 |
| 371 | 0.02 | 0.02 | 0.92 |
| 372 | 0.02 | 0.02 | 0.43 |
| 373 | 0.02 | 0.02 | 0.48 |
| 374 | 0.02 | 0.02 | 0.87 |
| 375 | 0.02 | 0.02 | 1.73 |
| 376 | 0.02 | 0.02 | 1.33 |
| 377 | 0.02 | 0.02 | 0.34 |
| 378 | 0.02 | 0.02 | 1.12 |
| 379 | 0.03 | 0.02 | 2.73 |
| 380 | 0.03 | 0.02 | 2.17 |
| 381 | 0.02 | 0.02 | 1.31 |
| 382 | 0.04 | 0.02 | 2.49 |
| 383 | 0.02 | 0.02 | 0.53 |
| 384 | 0.02 | 0.02 | 0.59 |
| 385 | 0.02 | 0.02 | 1.63 |
| 386 | 0.02 | 0.02 | 0.90 |
| 387 | 0.03 | 0.02 | 1.89 |
| 388 | 0.02 | 0.02 | 3.93 |
| 389 | 0.02 | 0.02 | 0.79 |
| 390 | 0.02 | 0.02 | 0.87 |
| 391 | 0.03 | 0.02 | 0.61 |
| 392 | 0.23 | 0.02 | 0.83 |
| 393 | 6.67 | 0.047 | 1.69 |
| 394 | 0.15 | 0.02 | 0.50 |
| 395 | 0.06 | 0.02 | 2.81 |
| 396 | 0.03 | 0.02 | 2.53 |
| 397 | 0.02 | 0.02 | 0.26 |
| 398 | 0.02 | 0.02 | 0.59 |
| 399 | 0.07 | 0.02 | 3.00 |
| 400 | 0.02 | 0.02 | 0.49 |
| 401 | 0.02 | 0.02 | 0.38 |
| 402 | 0.02 | 0.02 | 0.96 |
| 403 | 0.02 | 0.02 | 0.67 |
| 404 | 0.02 | 0.02 | 1.53 |
| 405 | 0.05 | 0.02 | 2.56 |
| 406 | 0.02 | 0.02 | 1.78 |
| 407 | 0.02 | 0.02 | 1.46 |
| 408 | 0.02 | 0.02 | 0.51 |
| 409 | 0.02 | 0.02 | 0.67 |
| 410 | 0.02 | 0.02 | 0.65 |
| 411 | 0.02 | 0.02 | 0.76 |
| 412 | 0.02 | 0.02 | 1.39 |
| 413 | 0.02 | 0.02 | 1.31 |
| 414 | 0.03 | 0.02 | 0.94 |
| 415 | 0.02 | 0.02 | 0.30 |
| 416 | 0.04 | 0.02 | 3.98 |
| 417 | 0.03 | 0.02 | 2.65 |
| 418 | 0.12 | 0.02 | 5.26 |
| 419 | 0.02 | 0.02 | 0.54 |
| 420 | 0.02 | 0.02 | 0.19 |
| 421 | 0.02 | 0.02 | 0.51 |
| 422 | 0.02 | 0.02 | 0.42 |
| 423 | 0.72 | 0.02 | 0.71 |
| 424 | 20.00 | 0.0455 | 3.82 |
| 425 | 0.15 | 0.02 | 0.21 |
| 426 | 0.11 | 0.02 | 2.06 |

### Preparation of Fluorescent Probe (FAM Probe)

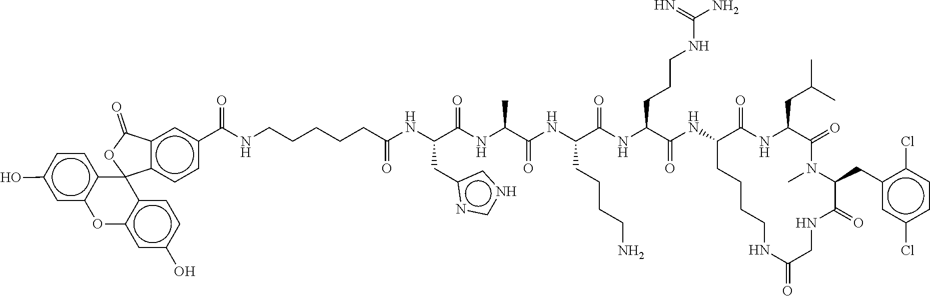

The fluorescent probe was synthesized via solid phase peptide synthesis followed by cyclization, fluorescent labeling, and deprotection in solution.

To load Fmoc-Glycine onto ˜50 mg of CTC resin, Fmoc-Glycine (G), CAS #29022-11-5, (4 equiv.) was dissolved in 1.0 mL of anhydrous NMP. Neat DIEA (8 equiv.) was added to the Fmoc-amino acid solution. The solution was dispensed in a peptide reactor vessel containing 50 mg of 2-chlorotrityl chloride (CTC) resin and was agitated for 2 hours at rt. The amino acid solution was drained then the resin was washed with 1.0 mL DMF three times. Unreacted CTC resin was capped with 1.0 mL solution of methanol:DMF (50:50), and DIEA (8 equiv.) for 10 min at rt. The methanol solution was drained then the resin was washed with 1.0 mL DMF three times. To remove Fmoc, A mixture of piperidine:DMF (20:80, 1 mL) was added to the resin and agitated for 10 to 15 min at rt. The piperidine solution was drained and then the resin was washed with 1.0 mL DMF three times.

A solution of Fmoc-L-2,5-dichlorophenylalanine-OH (25C₁F), CAS #1260614-80-9, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 10 to 15 min at rt. The piperidine solution was drained and then the resin was washed with 1.0 mL DMF three times.

To N-Methylate the amine of 25ClF, 2,6-lutidine (6 equiv.) dissolved in 0.5 mL of anhydrous DCE was added to the resin. 2-nitrobenzenesulfonyl chloride (5 equiv.) dissolved in 0.5 mL anhydrous toluene was added to the resin and then was agitated at 40 to 45° C. for 10 to 15 min. The mixture was drained, then the resin was washed with 1.0 mL of anhydrous toluene three times. The method was repeated twice. Triphenylphosphine (10 equiv.) dissolved in 0.7 mL anhydrous toluene was added to the resin. Dry methanol (MeOH), (20 equiv.) was added to the resin. Azodicarboxylate (10 equiv.) was added to the resin and the mixture was agitated at 45° C. for 30 min. The mixture was drained and the resin was washed with 1.0 mL of anhydrous DMF three times. Alkylation was repeated twice. The nosyl group was then deprotected. A solution of 2-mercaptoethanol (5 equiv.) and 1,8-Diazabicyclo[5.4.0]undec-7-ene (5 equiv.) in 1.0 mL NMP was added to the resin and the mixture was agitated at 45° C. for 10 min. The mixture was drained and then the resin was washed with 1.0 mL of anhydrous DMF three times. Deprotection was repeated twice.

Fmoc-L-Leucine-OH (L), CAS #35661-60-0 (12 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-L-Lysine(Mtt)-OH (KMtt), CAS #167393-62-6, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-L-Arginine(Pbf)-OH (RPbf), CAS #154445-77-9, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-L-Lysine(Boc)-OH (KBoc), CAS #71989-26-9, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-L-Alanine-OH (A), CAS #35661-39-3, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-L-Histidine(Trt)-OH (HTrt), CAS #109425-51-6, (4 equiv.), HATU (4 equiv.), and DIEA (8 equiv.) in 1.0 mL of anhydrous NMP was prepared. The mixture was allowed to pre-activate at rt for 5 min, and then was added to the resin and agitated at 35° C. for 30 min. The mixture was drained then the resin was washed with 1.0 mL of DMF three times. To remove Fmoc, A mixture of piperidine:DMF (20:80, 1 mL) was added to the resin and agitated for 15 min at rt. The piperidine solution was drained and then the resin was washed with 1.0 mL DMF three times.

Fmoc-6-aminohexanoic acid (Ahx), CAS #88574-06-5, (4 equiv.), HATU (4 equiv.), and DIEA (8 equiv) in 1.0 mL of anhydrous NMP was prepared. The mixture was allowed to pre-activate at rt for 5 min, and then was added to the resin and agitated at 35° C. for 30 min. The mixture was drained and then the resin was washed with 1.0 mL of DMF three times.

To cleave peptide from CTC resin and simultaneously deprotect the Mtt protecting group, approximately 2 mL of a solution of 24% HFIP, 2% TIPS, in DCM was added to the polystyrene resin in a solid phase reaction vessel. The contents were shaken for 1 hour. The cleavage solution was filtered into a 50 mL conical vial. The cleaved resin was washed with an additional 2 mL of DCM and the wash was collected in the conical vial. The solution was evaporated in a Genevac. The linear peptide was purified via reverse-phase HPLC using an Acetonitrile/Water gradient with 0.05% formic acid and the purified fractions were pooled and lyophilized to yield white powder of intermediate X (M/z observed=1968.65 [M+H]⁺).

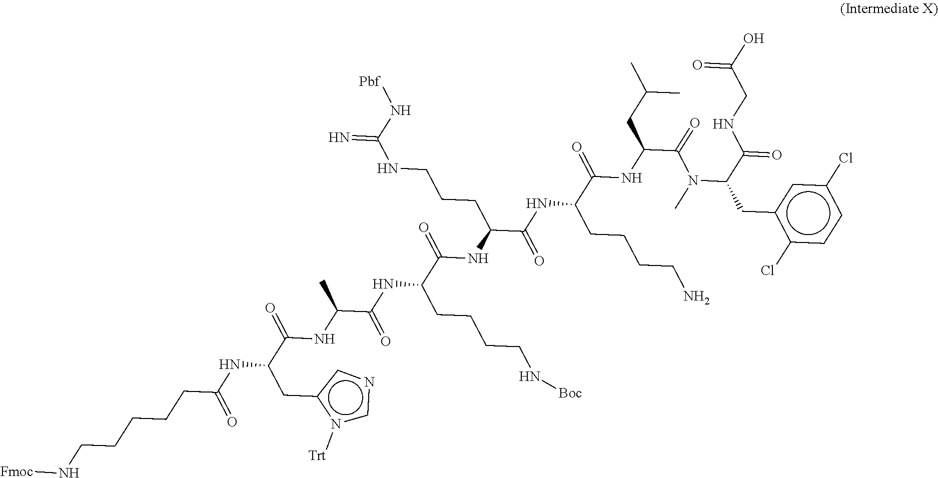

The linear intermediate X (˜15 mg) was cyclized using a medium volume, T3P solution cyclization method. The deprotected and purified linear product was transferred to a 50 mL conical vial and dissolved in 1 mL NMP followed by the addition of DIEA (0.5 mL) and DCM (35 mL). T3P (3 eqv) was added to the solution and the reaction pH was adjusted to pH 9 via dropwise addition of DIEA. The closed conical vial was agitated at rt for 2 hours at 150 rotations per minute. The solution was concentrated at 45° C. under reduced pressure in a Genevac system. The Fmoc group was then removed with the addition of a 10% of KOH/Water solution (5 mL) heated at 70° C. for 30 min. The resulting LCMS trace revealed that the trityl group had been unexpectedly removed during the cyclization and Fmoc-deprotection steps. The cyclic peptide was then purified via reverse phase HPLC using an Acetonitrile/Water gradient with 0.05% formic acid. The purified fractions were pooled and lyophilized to yield intermediate Y (M/z observed=1485.94 [M+Z]⁺).

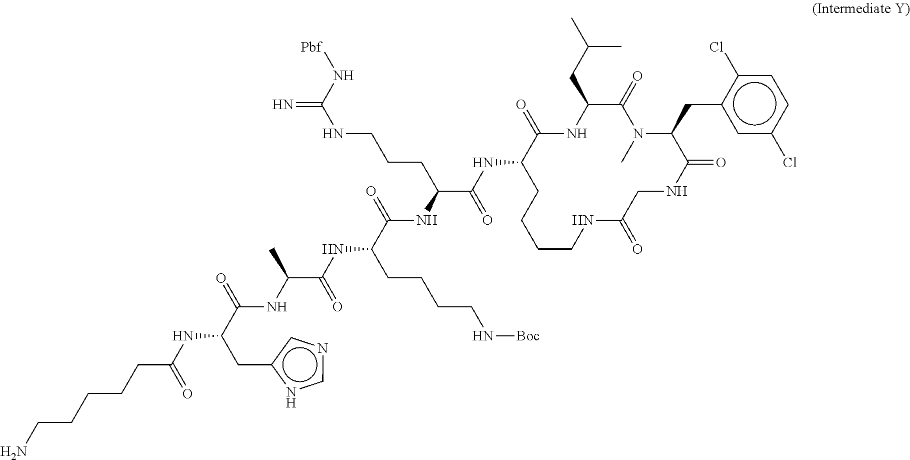

The probe was fluorescently labeled via a peptide coupling in solution. A solution of 5-carboxyfluorescein (CAS #76823-03-5, FAM) (4 equiv.), EDC (4 equiv.), HOAt (3.9 equiv.) and DIEA (8 equiv.) in 1.0 mL of anhydrous DCM was prepared. The mixture was allowed to pre-activate at rt for 5 min. Intermediate Y was added to the coupling solution, and the reaction was agitated at rt until starting material was not observed by LCMS, resulting in the formation of Intermediate Z (M/z observed=1844.29 [M+Z]⁺).

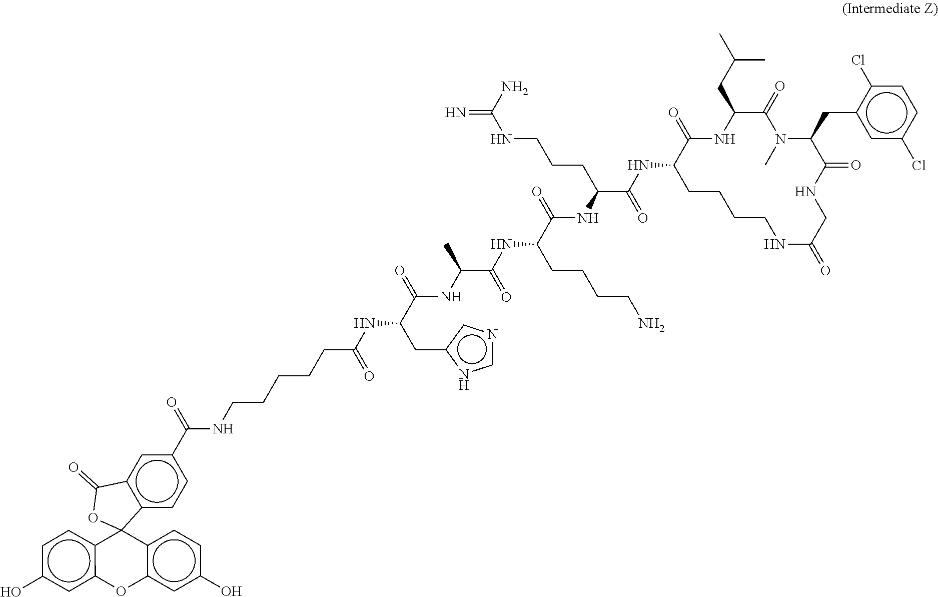

The Boc and Pbf protecting groups were removed from the cyclic intermediate Z by dissolving the cyclic peptide in a 1 mL solution of 90% TFA, 5% TIPS, 5% DCM and agitating for 1 hour. The reaction was monitored by LCMS for the disappearance of starting material. Upon completion, the reaction was concentrated. The crude material was co-evaporated with DCE (5 mL×2), and then purified via reverse phase-HPLC to yield fluorescent probe (FAM probe) (M/z observed=1492.14 [M+Z]+, 0.7 mg, 99% purity by HPLC).

### 2. MIT Proliferation Assay

MTT proliferation assay was used to determine the 50% growth inhibition (GI₅₀) of disclosed compounds. 5×10³ cells were seeded into 96 well plates. 24 hours later, cells were dosed with compound in an 8- or 10-point 1:3 serial dilution starting at 10 μM. Cells were exposed to compound for a sufficient time to allow 3-4 cell doublings (3 days (WI-38); 5 days (NCI-H1048 and OVCAR3)). Roscovitine and staurosporine were used as plate controls. At the end of the compound incubation, MTT reagent (TACS MTT Cell Proliferation Assay R&D Systems Catalog #4890-025-K) was added and assay carried out. Results are summarized in Table 6

TABLE 6
Cellular Activity Data of Exemplary Compounds

| Example | H1048 GI50 | OVCAR3 GI50 | WI-38 GI50 |
|---|---|---|---|
| Number | (μM) | (μM) | (μM) |
| 1 | 4.00 | 6.61 | 14.59 |
| 2 | 0.38 | 0.41 | 20.00 |
| 3 | 0.46 | 0.72 | 20.00 |
| 4 | 0.12 | 0.06 | 20.00 |
| 5-P1 | 0.39 | 0.69 | 20.00 |
| 5-P2 | 0.29 | 0.56 | 20.00 |
| 6-P1 | 0.05 | 0.09 | 16.37 |
| 6-P2 | 0.04 | 0.07 | 15.60 |
| 7 | 0.02 | 0.05 | 20.00 |
| 8 | 0.02 | 0.03 | 20.00 |
| 9 | 0.08 | 0.11 | 20.00 |
| 10 | 0.02 | 0.03 | 20.00 |
| 11 | 0.08 | 0.21 | 20.00 |
| 12 | 0.03 | 0.04 | 20.00 |
| 13 | 0.07 | 0.24 | 20.00 |
| 14 | 0.10 | 0.20 | 20.00 |
| 15 | 0.18 | 0.55 | 7.85 |
| 16-P1 | 0.34 | 0.88 | 20.00 |
| 16-P2 | 0.35 | 0.99 | 20.00 |
| 17 | 0.24 | 0.56 | 17.94 |
| 18 | 0.17 | 0.34 | 9.03 |
| 19 | 0.55 | 1.76 | 20.00 |
| 20 | 0.02 | 0.02 | 11.06 |
| 21 | 0.01 | 0.02 | 6.85 |
| 22 | 1.10 | 1.80 | 20.00 |
| 23 | 0.18 | 0.21 | 20.00 |
| 24 | 0.09 | 0.17 | 20.00 |
| 25 | 0.03 | 0.04 | 8.07 |
| 26 | 0.11 | 0.17 | 8.74 |
| 27 | 0.09 | 0.37 | 11.41 |
| 28 | 0.08 | 0.17 | 20.00 |
| 29 | 0.04 | 0.07 | 20.00 |
| 30 | 0.13 | 0.14 | 20.00 |
| 31 | 0.08 | 0.15 | 20.00 |
| 32 | 0.14 | 0.25 | 11.77 |
| 33 | 0.09 | 0.17 | 15.55 |
| 34 | 0.06 | 0.11 | 13.07 |
| 35 | 0.17 | 0.35 | 12.41 |
| 36 | 0.11 | 0.35 | 15.97 |
| 37 | 0.04 | 0.05 | 20.00 |
| 38 | 0.06 | 0.16 | 20.00 |
| 39 | 0.09 | 0.21 | 20.00 |
| 40 | 0.06 | 0.05 | 15.39 |
| 41 | 0.10 | 0.13 | 20.00 |
| 42 | 0.07 | 0.21 | 20.00 |
| 43 | 0.03 | 0.04 | 8.57 |
| 44 | 0.02 | 0.04 | 6.60 |
| 45 | 0.02 | 0.04 | 20.00 |
| 46 | 0.72 | 0.79 | 20.00 |
| 47 | 0.03 | 0.13 | 12.93 |
| 48 | 0.04 | 0.05 | 15.80 |
| 49 | 0.01 | 0.01 | 11.54 |
| 50 | 0.01 | 0.01 | 20.00 |
| 51 | 0.01 | 0.01 | 18.81 |
| 52 | 0.01 | 0.01 | 13.14 |
| 53 | 0.18 | 0.18 | 4.50 |
| 54 | 0.47 | 0.70 | 20.00 |
| 55 | 0.56 | 0.71 | 20.00 |
| 56-P1 | 0.47 | 1.02 | 5.76 |
| 56-P2 | 0.19 | 0.43 | 3.76 |
| 57-P1 | 0.01 | 0.01 | 13.77 |
| 57-P2 | 0.01 | 0.01 | 16.87 |
| 58 | 0.01 | 0.01 | 20.00 |
| 59-P1 | 0.02 | 0.02 | 20.00 |
| 59-P2 | 0.06 | 0.05 | 20.00 |
| 60 | 0.01 | 0.01 | 5.72 |
| 61 | 0.02 | 0.04 | 20.00 |
| 62 | 0.06 | 0.07 | 20.00 |
| 63 | 0.11 | 0.24 | 3.17 |
| 64 | 0.51 | 1.24 | 20.00 |
| 65 | 0.01 | 0.01 | 20.00 |
| 66 | 0.01 | 0.01 | 12.89 |
| 67 | 0.01 | 0.01 | 20.00 |
| 68 | 0.01 | 0.01 | 12.81 |
| 69 | 0.01 | 0.01 | 17.57 |
| 70 | 0.01 | 0.01 | 10.11 |
| 71 | 0.07 | 0.09 | 13.82 |
| 72 | 0.04 | 0.06 | 14.87 |
| 73 | 0.04 | 0.06 | 3.65 |
| 74 | 0.11 | 0.17 | 20.00 |
| 75 | 0.01 | 0.02 | 5.89 |
| 76 | 0.08 | 0.12 | 20.00 |
| 77 | 0.06 | 0.07 | 20.00 |
| 78 | 0.03 | 0.04 | 4.52 |
| 79 | 0.03 | 0.04 | 20.00 |
| 80 | 0.03 | 0.03 | 11.63 |
| 81 | 0.04 | 0.05 | 4.30 |
| 82 | 0.03 | 0.07 | 20.00 |
| 83 | 0.02 | 0.01 | 20.00 |
| 84 | 0.02 | 0.01 | 20.00 |
| 85 | 0.02 | 0.02 | 20.00 |
| 86 | 0.02 | 0.02 | 20.00 |
| 87 | 0.05 | 0.06 | 20.00 |
| 88 | 0.03 | 0.03 | 7.64 |
| 89 | 0.31 | 0.38 | 4.34 |
| 90 | 0.26 | 0.23 | 4.13 |
| 91 | 0.23 | 0.26 | 20.00 |
| 92 | 0.01 | 0.01 | 20.00 |
| 93 | 0.01 | 0.01 | 20.00 |
| 94 | 0.01 | 0.01 | 20.00 |
| 95 | 0.01 | 0.02 | 20.00 |
| 96 | 0.01 | 0.01 | 8.92 |
| 97 | 0.02 | 0.03 | 20.00 |
| 98 | 0.02 | 0.02 | 16.45 |
| 99 | 0.02 | 0.03 | 20.00 |
| 100 | 0.02 | 0.02 | 20.00 |
| 101 | 0.02 | 0.03 | 20.00 |
| 102-P1 | 0.35 | 0.81 | 20.00 |
| 102-P2 | 0.08 | 0.13 | 20.00 |
| 103 | 0.03 | 0.05 | 20.00 |
| 104 | 0.16 | 0.26 | 20.00 |
| 105 | 0.02 | 0.03 | 18.78 |
| 106 | 0.41 | 0.54 | 6.39 |
| 107-P1 | 0.70 | 1.01 | 5.29 |
| 107-P2 | 0.19 | 0.32 | 20.00 |
| 108 | 0.02 | 0.03 | 20.00 |
| 109 | 0.02 | 0.02 | 9.52 |
| 110 | 0.01 | 0.01 | 4.80 |
| 111 | 0.01 | 0.01 | 8.13 |
| 112 | 0.01 | 0.01 | 20.00 |
| 113 | 0.01 | 0.01 | 7.24 |
| 114 | 0.01 | 0.01 | 7.19 |
| 115 | 0.07 | 0.06 | 11.00 |
| 116 | 0.04 | 0.05 | 20.00 |
| 117-P1 | 0.23 | 0.18 | 20.00 |
| 117-P2 | 0.09 | 0.06 | 20.00 |
| 118-P1 | 0.18 | 0.17 | 20.00 |
| 118-P2 | 0.05 | 0.05 | 20.00 |
| 119 | 0.25 | 0.30 | 8.71 |
| 120 | 0.10 | 0.13 | 9.28 |
| 121 | 0.04 | 0.07 | 7.61 |
| 122 | 0.05 | 0.05 | 20.00 |
| 123 | 0.02 | 0.02 | 20.00 |
| 124 | 0.02 | 0.02 | 20.00 |
| 125 | 0.49 | 0.55 | 20.00 |
| 126 | 0.38 | 0.31 | 20.00 |
| 127 | 0.03 | 0.03 | 20.00 |
| 128 | 0.03 | 0.03 | 20.00 |
| 129 | 1.46 | 2.58 | 6.88 |
| 130 | 0.70 | 1.55 | 8.99 |
| 131 | 0.29 | 0.40 | 20.00 |
| 132 | 0.57 | 0.38 | 20.00 |
| 133 | 0.34 | 0.26 | 20.00 |
| 134 | 0.03 | 0.04 | 20.00 |
| 135 | 0.03 | 0.02 | 20.00 |
| 136-P1 | 0.83 | 1.77 | 3.96 |
| 136-P2 | 0.45 | 0.64 | 6.67 |
| 137 | 0.60 | 0.76 | 4.58 |
| 138 | 0.20 | 0.28 | 6.67 |
| 139 | 0.07 | 0.15 | 6.42 |
| 140 | 0.05 | 0.13 | 9.25 |
| 141 | 0.09 | 0.19 | 3.39 |
| 142 | 0.11 | 0.19 | 10.20 |
| 143 | 0.01 | 0.02 | 5.18 |
| 144 | 0.07 | 0.07 | 20.00 |
| 145 | 0.07 | 0.08 | 10.14 |
| 146 | 0.02 | 0.02 | 20.00 |
| 147 | 0.01 | 0.01 | 1.24 |
| 148 | 0.01 | 0.01 | 20.00 |
| 149 | 0.03 | 0.04 | 20.00 |
| 150 | 0.02 | 0.04 | 20.00 |
| 151 | 0.01 | 0.01 | 20.00 |
| 152 | 0.50 | 0.45 | 20.00 |
| 153 | 0.11 | 0.14 | 20.00 |
| 154 | 0.02 | 0.02 | 20.00 |
| 155 | 0.01 | 0.02 | 20.00 |
| 156-P1 | 0.48 | 0.53 | 20.00 |
| 156-P2 | 0.09 | 0.10 | 20.00 |
| 157 | 0.08 | 0.14 | 20.00 |
| 158 | 0.02 | 0.02 | 20.00 |
| 159 | 0.01 | 0.02 | 9.45 |
| 160 | 0.02 | 0.02 | 11.46 |
| 161 | 0.02 | 0.02 | 20.00 |
| 162 | 0.01 | 0.01 | 20.00 |
| 163 | 0.01 | 0.01 | 20.00 |
| 164 | 0.03 | 0.03 | 20.00 |
| 165 | 0.34 | 1.67 | 20.00 |
| 166 | 0.04 | 0.10 | 20.00 |
| 167 | 0.01 | 0.01 | 19.74 |
| 168 | 0.01 | 0.01 | 13.10 |
| 169 | 0.02 | 0.03 | 20.00 |
| 170 | 0.13 | 0.20 | 20.00 |
| 171 | 0.15 | 0.21 | 20.00 |
| 172 | 0.03 | 0.06 | 13.70 |
| 173 | 0.01 | 0.01 | 12.74 |
| 174 | 0.01 | 0.01 | 6.98 |
| 175 | 0.01 | 0.01 | 6.85 |
| 176 | 0.01 | 0.01 | 5.48 |
| 177 | 0.01 | 0.01 | 8.96 |
| 178 | 1.18 | 2.57 | 20.00 |
| 179 | 0.01 | 0.03 | 11.61 |
| 180 | 0.73 | 1.22 | 20.00 |
| 181 | 0.24 | 0.42 | 20.00 |
| 182 | 0.06 | 0.13 | 20.00 |
| 183 | 0.01 | 0.02 | 5.07 |
| 184 | 0.01 | 0.01 | 3.65 |
| 185 | 0.01 | 0.01 | 10.35 |
| 186 | 0.01 | 0.01 | 8.68 |
| 187 | 0.01 | 0.01 | 8.66 |
| 188 | 0.01 | 0.02 | 3.08 |
| 189 | 0.13 | 0.08 | 3.67 |
| 190 | 0.01 | 0.01 | 1.98 |
| 191 | 0.01 | 0.01 | 2.05 |
| 192 | 0.02 | 0.02 | 3.51 |
| 193 | 0.01 | 0.02 | 7.58 |
| 194 | 0.01 | 0.01 | 5.61 |
| 195 | 0.01 | 0.01 | 20.00 |
| 196 | 0.01 | 0.01 | 20.00 |
| 197 | 0.05 | 0.10 | 20.00 |
| 198 | 0.05 | 0.05 | 20.00 |
| 199 | 0.03 | 0.05 | 20.00 |
| 200 | 0.04 | 0.03 | 20.00 |
| 201 | 0.06 | 0.06 | 20.00 |
| 202 | 0.04 | 0.06 | 9.11 |
| 203 | 0.06 | 0.05 | 3.70 |
| 204 | 0.01 | 0.01 | 20.00 |
| 205 | 0.02 | 0.03 | 20.00 |
| 206 | 0.01 | 0.02 | 20.00 |
| 207 | 0.03 | 0.04 | 4.94 |
| 208 | 0.83 | 1.96 | 20.00 |
| 209 | 0.01 | 0.02 | 20.00 |
| 210 | 0.10 | 0.11 | 20.00 |
| 211 | 0.02 | 0.03 | 20.00 |
| 212 | 0.08 | 0.08 | 20.00 |
| 213 | 0.01 | 0.01 | 20.00 |
| 214 | 0.01 | 0.01 | 20.00 |
| 215 | 0.01 | 0.01 | 20.00 |
| 216 | 0.01 | 0.01 | 15.55 |
| 217 | 0.01 | 0.02 | 20.00 |
| 218 | 0.23 | 0.39 | 20.00 |
| 219 | 0.04 | 0.08 | 14.06 |
| 220 | 0.03 | 0.07 | 5.20 |
| 221 | 0.02 | 0.03 | 16.64 |
| 222 | 0.01 | 0.01 | 20.00 |
| 223 | 0.01 | 0.01 | 7.76 |
| 224 | 0.01 | 0.01 | 12.41 |
| 225 | 0.02 | 0.03 | 8.53 |
| 226 | 0.02 | 0.03 | 8.39 |
| 227 | 0.02 | 0.04 | 14.09 |
| 228 | 0.06 | 0.08 | 9.54 |
| 229 | 0.01 | 0.02 | 20.00 |
| 230 | 0.04 | 0.09 | 5.05 |
| 231 | 0.02 | 0.04 | 13.36 |
| 232 | 0.05 | 0.08 | 11.61 |
| 233 | 0.03 | 0.03 | 15.15 |
| 234 | 0.01 | 0.01 | 10.66 |
| 235 | 0.10 | 0.09 | 13.15 |
| 236 | 0.01 | 0.02 | 7.33 |
| 237 | 0.01 | 0.04 | 20.00 |
| 238 | 0.01 | 0.02 | 17.27 |
| 239 | 0.02 | 0.02 | 14.31 |
| 240 | 0.01 | 0.01 | 8.73 |
| 241 | 0.0 | 0.01 | 12.45 |
| 242 | 0.03 | 0.02 | 20.00 |
| 243 | 0.05 | 0.04 | 13.29 |
| 244 | 0.10 | 0.04 | 20.00 |
| 245 | 0.08 | 0.02 | 20.00 |
| 246 | 0.03 | 0.01 | 20.00 |
| 247 | 0.05 | 0.04 | 20.00 |
| 248 | 0.04 | 0.02 | 20.00 |
| 249 | 0.01 | 0.01 | 20.00 |
| 250 | 0.03 | 0.03 | 5.55 |
| 251 | 0.01 | 0.01 | 13.92 |
| 252 | 0.01 | 0.02 | 20.00 |
| 253 | 0.04 | 0.02 | 9.93 |
| 254 | 0.02 | 0.02 | 20.00 |
| 255 | 0.04 | 0.03 | 15.76 |
| 256 | 0.01 | 0.01 | 13.19 |
| 257 | 0.01 | 0.01 | 13.26 |
| 258 | 0.07 | 0.07 | 14.19 |
| 259 | 0.11 | 0.11 | 10.43 |
| 260 | 0.04 | 0.04 | 6.39 |
| 261 | 0.01 | 0.01 | 14.10 |
| 262 | 0.01 | 0.02 | 16.21 |
| 263 | 0.03 | 0.04 | 20.00 |
| 264 | 0.04 | 0.03 | 20.00 |
| 265 | 0.01 | 0.02 | 20.00 |
| 266 | 0.03 | 0.05 | 20.00 |
| 267 | 0.05 | 0.04 | 20.00 |
| 268 | 0.02 | 0.01 | 20.00 |
| 269 | 0.03 | 0.03 | 20.00 |
| 270 | 0.08 | 0.07 | 13.38 |
| 271 | 0.01 | 0.01 | 20.00 |
| 272 | 0.09 | 0.43 | 20.00 |
| 273 | 0.03 | 0.04 | 20.00 |
| 274 | 0.01 | 0.01 | 10.48 |
| 275 | 0.01 | 0.01 | 20.00 |
| 276 | 0.02 | 0.02 | 23.38 |
| 277 | 0.33 | 0.36 | 3.82 |
| 278 | 0.01 | 0.01 | 20.00 |
| 279 | 0.08 | 0.09 | 20.00 |
| 280 | 0.01 | 0.01 | 20.00 |
| 281 | 0.01 | 0.02 | 20.00 |
| 282 | 0.01 | 0.01 | 20.00 |
| 283 | 0.01 | 0.01 | 14.18 |
| 284 | 0.01 | 0.01 | 20.00 |
| 285 | 0.05 | 0.03 | 20.00 |
| 286 | 0.06 | 0.05 | 6.50 |
| 287 | 0.04 | 0.04 | 20.00 |
| 288 | 0.02 | 0.02 | 13.81 |
| 289 | 0.01 | 0.01 | 3.78 |
| 290 | 0.08 | 0.15 | 20.00 |
| 291 | 0.12 | 0.15 | 20.00 |
| 292 | 0.07 | 0.11 | 20.00 |
| 293 | 0.05 | 0.09 | 20.00 |
| 294 | 0.08 | 0.13 | 20.00 |
| 295 | 0.08 | 0.16 | 20.00 |
| 296 | 0.07 | 0.10 | 20.00 |
| 297 | 0.02 | 0.03 | 9.15 |
| 298 | 0.01 | 0.02 | 20.00 |
| 299 | 0.01 | 0.01 | 14.29 |
| 300 | 0.03 | 0.04 | 20.00 |
| 301 | 0.02 | 0.02 | 20.00 |
| 302 | 0.14 | 0.12 | 20.00 |
| 303 | 0.01 | 0.01 | 20.00 |
| 304 | 0.01 | 0.01 | 20.00 |
| 305 | 0.01 | 0.02 | 19.20 |
| 306 | 0.01 | 0.01 | 20.00 |
| 307 | 0.01 | 0.01 | 20.00 |
| 308 | 0.01 | 0.01 | 20.00 |
| 309 | 0.01 | 0.01 | 20.00 |
| 310 | 0.01 | 0.01 | 20.00 |
| 311 | 0.02 | 0.03 | 20.00 |
| 312 | 0.02 | 0.03 | 20.00 |
| 313 | 0.01 | 0.01 | 36.59 |
| 314 | 0.01 | 0.01 | 20.00 |
| 315 | 0.02 | 0.02 | 20.00 |
| 316 | 0.01 | 0.01 | 20.00 |
| 317 | 0.01 | 0.01 | 20.00 |
| 318 | 0.01 | 0.01 | 9.92 |
| 319 | 0.01 | 0.02 | 20.00 |
| 320 | 0.01 | 0.02 | 15.50 |
| 321 | 0.01 | 0.01 | 89.76 |
| 322 | 0.03 | 0.06 | 20.00 |
| 323 | 0.01 | 0.01 | 20.00 |
| 324 | 0.04 | 0.08 | 20.00 |
| 325 | 0.01 | 0.01 | 20.00 |
| 326 | 0.01 | 0.01 | 20.00 |
| 327 | 0.01 | 0.01 | 83.88 |
| 328 | 0.01 | 0.01 | 20.00 |
| 329 | 0.01 | 0.01 | 20.00 |
| 330 | 0.01 | 0.01 | 18.46 |
| 331 | 0.06 | 0.05 | 20.00 |
| 332 | 0.01 | 0.01 | 20.00 |
| 333 | 0.01 | 0.01 | 20.00 |
| 334 | 0.01 | 0.01 | 20.00 |
| 335 | 0.01 | 0.01 | 4.75 |
| 336 | 0.01 | 0.01 | 2.48 |
| 337 | 0.01 | 0.01 | 18.7 |
| 338 | 0.02 | 0.02 | 18.9 |
| 339 | 0.01 | 0.01 | 5.62 |
| 340 | 0.02 | 0.02 | 17.3 |
| 341 | 0.01 | 0.01 | 2.44 |
| 342 | 0.01 | 0.01 | 20 |
| 343 | 0.01 | 0.01 | 20 |
| 344 | 0.01 | 0.02 | 20 |
| 345 | 0.02 | 0.04 | 20 |
| 346 | 0.01 | 0.01 | 3.68 |
| 347 | 0.01 | 0.01 | 1.81 |
| 348 | 0.01 | 0.01 | 18 |
| 349 | 0.01 | 0.01 | 20 |
| 350 | 0.01 | 0.01 | 20 |
| 351 | 0.01 | 0.01 | 1.78 |
| 352 | 0.01 | 0.01 | 20 |
| 353 | 0.01 | 0.01 | 3.49 |
| 354 | 0.01 | 0.02 | 20 |
| 355 | 0.01 | 0.01 | 2.77 |
| 356 | 0.01 | 0.01 | 3.43 |
| 357 | 0.02 | 0.05 | 3.14 |
| 358 | 0.01 | 0.01 | 2.13 |
| 359 | 0.01 | 0.01 | 4.56 |
| 360 | 0.01 | 0.01 | 2.72 |
| 361 | 0.03 | 0.01 | 14.3 |
| 362 | 0.01 | 0.01 | 1.28 |
| 363 | 0.01 | 0.01 | 1.43 |
| 364 | 0.01 | 0.01 | 3.25 |
| 365 | 0.01 | 0.01 | 3.18 |
| 366 | 0.02 | 0.02 | 20 |
| 367 | 0.02 | 0.01 | 14.7 |
| 368 | 0.01 | 0.01 | 15.2 |
| 369 | 0.02 | 0.02 | 4.54 |
| 370 | 0.02 | 0.02 | 1.21 |
| 371 | 0.01 | 0.01 | 1.44 |
| 372 | 0.01 | 0.01 | 2.84 |
| 373 | 0.01 | 0.01 | 20 |
| 374 | 0.01 | 0.01 | 2.22 |
| 375 | 0.01 | 0.02 | 16.8 |
| 376 | 0.01 | 0.01 | 3.45 |
| 377 | 0.01 | 0.01 | 8.16 |
| 378 | 0.01 | 0.02 | 20 |
| 379 | 0.03 | 0.02 | 20 |
| 380 | 0.02 | 0.03 | 1.17 |
| 381 | 0.02 | 0.04 | 2.3 |
| 382 | 0.09 | 0.04 | 20 |
| 383 | 0.01 | 0.01 | 2.7 |
| 384 | 0.01 | 0.01 | 2.21 |
| 385 | 0.03 | 0.02 | 20 |
| 386 | 0.03 | 0.02 | 20 |
| 387 | 0.03 | 0.02 | 3.08 |
| 388 | 0.02 | 0.02 | 3.5 |
| 389 | 0.02 | 0.01 | 17.6 |
| 390 | 0.03 | 0.02 | 9.24 |
| 391 | 0.01 | 0.01 | 7.82 |
| 392 | 0.17 | 0.17 | 14.70 |
| 393 | 1.79 | 1.31 | 13.70 |
| 394 | 0.09 | 0.05 | 20.00 |
| 395 | 0.02 | 0.01 | |
| 396 | 0.02 | 0.01 | |
| 397 | 0.01 | 0.01 | 3.56 |
| 398 | 0.14 | 0.19 | 3.64 |
| 399 | 0.22 | 0.11 | 3.65 |
| 400 | 0.01 | 0.01 | 4.61 |
| 401 | 0.01 | 0.01 | |
| 402 | 0.04 | 0.01 | 20.00 |
| 403 | 0.01 | 0.01 | 3.99 |
| 404 | 0.02 | 0.03 | 20.00 |
| 405 | 0.04 | 0.05 | 2.16 |
| 406 | 0.02 | 0.04 | 3.35 |
| 407 | 0.01 | 0.02 | 3.01 |
| 408 | 0.01 | 0.01 | 5.53 |
| 409 | 0.01 | 0.01 | 2.46 |
| 410 | 0.01 | 0.01 | 2.00 |
| 411 | 0.01 | 0.01 | 1.61 |
| 412 | 0.01 | 0.01 | 1.06 |
| 413 | 0.01 | 0.01 | 1.60 |
| 414 | 0.01 | 0.01 | 20.00 |
| 415 | 0.01 | 0.01 | 14.10 |
| 416 | 0.03 | 0.04 | 20.00 |
| 417 | 0.01 | 0.01 | 1.16 |
| 418 | 1.42 | 1.15 | 20.00 |
| 419 | 0.02 | 0.02 | 1.99 |
| 420 | 0.01 | 0.02 | 3.51 |
| 421 | 0.01 | 0.01 | 19.10 |
| 422 | 0.01 | 0.01 | 2.36 |
| 423 | 0.20 | 0.07 | 20.00 |
| 424 | 2.51 | 1.78 | 20.00 |
| 425 | 0.16 | 0.24 | 20.00 |
| 426 | 0.09 | 0.15 | 20.00 |

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A compound of Formula (I) 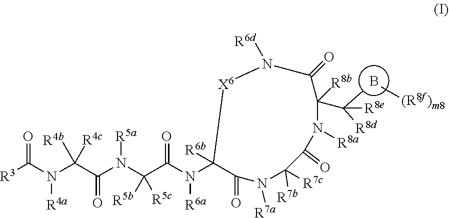
wherein
R³ is
(a) C₁₋₆ alkyl, C₂₋₆ alkenyl, C₂₋₆ alkynyl, or C₁₋₆ haloalkyl, each substituted with 0 to 5 R^{3a},
(b) C₃₋₁₂ cycloalkyl substituted with 0 to 5 R^{3b}, or
(c) heterocycloalkyl having 3 to 12 ring members and 1 to 4 heteroatoms, each independently N, O, or S, wherein the heterocycloalkyl is substituted with 0 to 5 R^{3c};
(g) heteroaryl having 5 to 6 ring members and 1 to 3 heteroatoms, each independently N, O, or S, wherein the heteroaryl is substituted with 0, 1, 2, 3, 4, or 5 R^{3g};
each R^{3a} is —OH, C₁₋₆ alkoxy, C₁₋₆ haloalkoxy, or —O—C(O)C₁₋₆ alkyl;
each R^{3b} is independently C₁₋₆ alkyl, C₂₋₆ alkenyl, C₂₋₆ alkynyl, —OH, C₁₋₆ alkoxy, halo, C₁₋₆ haloalkyl, C₁₋₆ haloalkoxy, or cyano;
each R^{3c} is independently C₁₋₆ alkyl, halo, C₁₋₆ haloalkyl, cyano, C₃₋₆ cycloalkyl, or oxo;
each R^{3g} is independently C₁₋₆ alkyl, halo, C₁₋₆ haloalkyl, or C₃₋₆ cycloalkyl;
R^{4a} is H or C₁₋₆ alkyl;
R^{4b} and R^{4c} are each independently H, C₁₋₆ alkyl, C₁₋₆ haloalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S;
alternatively, R^{4c} and R^{4a} together with the carbon and nitrogen to which each is attached combine to form a heterocycloalkyl having 3 to 6 ring members and 1 to 3 heteroatoms, each independently N, O, or S, wherein the heterocycloalkyl is substituted with 0 to 4 R^{4a1};
each R^{4a1} is independently C₁₋₆ alkyl, —OH, C₁₋₆ alkyl-OH, C₁₋₆ alkoxy, or halo;
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, —C₁₋₆ alkyl-C(O)NR^{5b1}R^{5b2}, —C₁₋₆ alkyl-C(O)OR^{5b1}, C₃₋₆ cycloalkyl, C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or
—C₁₋₆ alkyl-heteroaryl, wherein each heterocycloalkyl has 3 to 6 ring members and 1 to 3 heteroatoms, each independently N, O, or S, and each heteroaryl has 5 to 6 ring members and 1 to 3 heteroatoms, each independently N, O, or S, and wherein each cycloalkyl, heterocycloalkyl, phenyl, and heteroaryl is substituted with 0 to 3 R^{5b5};
each R^{5b1} and R^{5b2} are independently H, C₁₋₄ alkyl, or C₁₋₄ haloalkyl;
alternatively, R^{5b1} and R^{5b2} together with the nitrogen to which each is attached combine to form a heterocycloalkyl having 3 to 6 ring members and 0 to 1 additional heteroatoms, wherein the heterocycloalkyl is substituted with 0 to 2 R^{5b3};
each R^{5b3} and R^{5b5} is independently C₁₋₆ alkyl, halo, C₁₋₆ haloalkyl, or cyano;
X⁶ is C₆₋₇ alkylene or C₆₋₇ alkenylene;
R^{6a} is H, C₁₋₆ alkyl, C₁₋₆ deuteroalkyl, C₂₋₆ alkoxyalkyl, C₃₋₆ cycloalkyl, —C₁₋₆ alkyl-C₃₋₆ cycloalkyl, heterocycloalkyl, —C₁₋₆ alkyl-heterocycloalkyl, phenyl, —C₁₋₆ alkyl-phenyl, heteroaryl, or —C₁₋₆ alkyl-heteroaryl, wherein the heterocycloalkyl has 3 to 6 ring members and 1 to 3 heteroatoms, each independently N, O, or S, and the heteroaryl has 5 to 6 ring members and 1 to 3 heteroatoms, each independently N, O, or S;
R^{6b} and R^{6d} are each independently H or C₁₋₆ alkyl;
R^{7a} is H or C₁₋₆ alkyl;
R^{7b} and R^{7c} are each independently H, C₁₋₆ alkyl, C₂₋₆ alkenyl, C₁₋₆ haloalkyl, C₁₋₆ alkyl-OH, C₃₋₆ cycloalkyl, or —C₁₋₆ alkyl-C₃₋₆ cycloalkyl;
R^{8a} is H, C₁₋₆ alkyl, C₂₋₆ alkoxyalkyl, C₃₋₆ cycloalkyl or —C₁₋₆ alkyl-C₃₋₆ cycloalkyl;
R^{8b}, R^{8d}, and R^{8e} are each independently H or C₁₋₆ alkyl;
ring B is C₆₋₁₂ aryl or heteroaryl having 5 to 12 ring members and 1 to 6 heteroatoms, each independently N, O or S;
the subscript m8 is an integer from 0 to 5;
each R^{8f} is independently C₁₋₆ alkyl, C₂₋₆ alkenyl, C₂₋₆ alkynyl, C₁₋₆ alkoxy, C₁₋₆ deuteroalkoxy, halo, C₁₋₆ haloalkyl, cyano, —X^{8f}-cyano, C₃₋₁₂ cycloalkyl, —X^{8f}—C₃₋₆ cycloalkyl, —CH═CR^{8f1}R^{8f2}, heterocycloalkyl,
—X^{8f}-heterocycloalkyl, C₆₋₁₂ aryl, —X^{8f}—C₆₋₁₂ aryl, heteroaryl, or
—X^{8f}-heteroaryl, wherein each heterocycloalkyl has 3 to 12 ring members and 1 to 4 heteroatoms, each independently N, O, or S, and each heteroaryl has 5 to 10 ring members and 1 to 4 heteroatoms, each independently N, O, or S, wherein each alkynyl, cycloalkyl, heterocycloalkyl, aryl, and heteroaryl is substituted with 0 to 3 R^{8f3};
alternatively, two R^{8f} groups on adjacent ring vertices combine to form a C₃₋₆ cycloalkyl or a heterocycloalkyl having 3 to 6 ring members and 1 to 3 heteroatoms, each independently N, O or S, wherein the cycloalkyl or heterocycloalkyl is substituted with 0 to 3 R^{8f3};
each X^{8f} is independently C₁₋₆ alkylene, C₂₋₆ alkenylene, —O—C₁₋₆ alkylene, C(O), O, or S;
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3};
each R^{8f3} is independently C₁₋₆ alkyl, —Y⁸—C₁₋₆ alkyl, C₁₋₆ deuteroalkyl, —Y⁸—C₁₋₆ deuteroalkyl, —OH, —C₁₋₆ alkyl-OH, —Y⁸—C₁₋₆ alkyl-OH, —C₁₋₆ alkyl-Y⁸—C₁₋₆ alkyl, —(C₁₋₂ alkyl-O)₁₋₄—C₁₋₂ alkyl, C₁₋₆ alkoxy, halo, C₁₋₆ haloalkyl, —Y⁸—C₁₋₆ haloalkyl, cyano, —C₁₋₆ alkyl-cyano, —C₁₋₆ alkyl-NR^{8g}R^{8h}, oxo, C₃₋₆ cycloalkyl, —X^{8f3}—C₃₋₆ cycloalkyl, heterocycloalkyl, —X^{8f3}-heterocycloalkyl, phenyl, —X^{8f3}-phenyl, heteroaryl, or —X^{8f3}-heteroaryl, wherein each heterocycloalkyl has 3 to 12 members and 1 to 4 heteroatoms, each independently N, O, S, or S(O)₂, and each heteroaryl has 5 to 10 ring members and 1 to 4 heteroatoms, each independently N, O, or S, and wherein each heterocycloalkyl and heteroaryl are substituted with 0 to 2 C₁₋₄ alkyl or halo;
each X^{8f3} is independently C₁₋₆ alkylene, O, C(O), or S(O)₂;
each Y⁸ is independently C(O), C(O)O, N(R^{8f4})C(O), O, S, or S(O)₂;
each R^{8g} and R^{8h} is independently H, C₁₋₆ alkyl, or C₁₋₆ haloalkyl;
alternatively, two R^{8f3} groups on the same or adjacent ring vertices combine to form a C₃₋₆ cycloalkyl or a heterocycloalkyl having 3 to 6 ring members and 1 to 3 heteroatoms, each independently N, O or S, wherein nitrogen atom ring members in the heterocycloalkyl are substituted with 0 to 1 C₁₋₄ alkyl; and
each R^{8f4} is independently H or C₁₋₆ alkyl;
or a pharmaceutically acceptable salt thereof.

2.-3. (canceled)

4. The compound of claim 1, or the pharmaceutically acceptable salt thereof, wherein R³ is (a) C₁₋₆ alkyl, C₂₋₆ alkenyl, C₂₋₆ alkynyl, or C₁₋₆ haloalkyl substituted with 0 to 5 R^{3a}.

5.-6. (canceled)

7. The compound of claim 1, or the pharmaceutically acceptable salt thereof, wherein R³ is (b) C₃₋₇ cycloalkyl substituted with 0 to 3 R^{3b}.

8. The compound of claim 1, or the pharmaceutically acceptable salt thereof, wherein R³ is (b) C₃₋₄ cycloalkyl substituted with 0 to 2 R^{3b}.

9. The compound of claim 7, or the pharmaceutically acceptable salt thereof, wherein each R^{3b} is halo or C₁₋₄ haloalkyl.

10. The compound of claim 1, or the pharmaceutically acceptable salt thereof, wherein R³ is (c) heterocycloalkyl having 3 to 6 ring members and 1 to 3 heteroatoms, each independently N, O, or S, wherein the heterocycloalkyl is substituted with 0 to 5 R^{3c}.

11. The compound of claim 10, or the pharmaceutically acceptable salt thereof, wherein each R^{3c} is halo or C₁₋₄ haloalkyl.

12. (canceled)

13. The compound of claim 1, or the pharmaceutically acceptable salt thereof, wherein R³ is

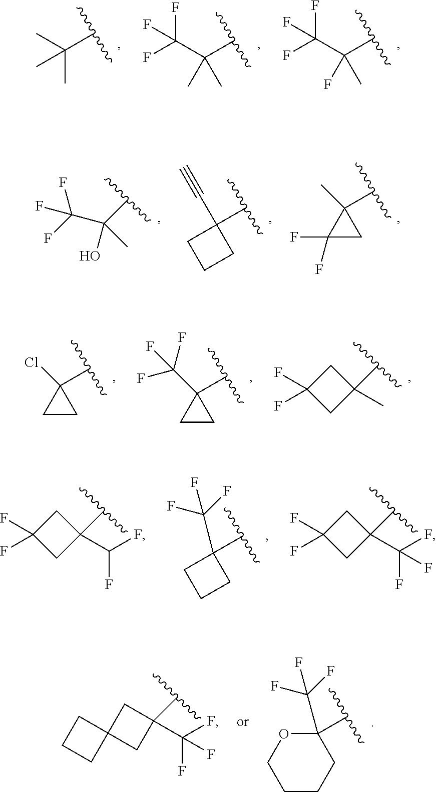

14. The compound of claim 1, or the pharmaceutically acceptable salt thereof, wherein
R^{4a}, R^{4b}, and R^{4c} are each independently H or C₁₋₆ alkyl;
alternatively, R^{4c} and R^{4a} together with the carbon and nitrogen to which each is attached combine to form a heterocycloalkyl having 4 to 6 ring members and 1 to 3 heteroatoms, each independently N, O, or S, wherein the heterocycloalkyl is substituted with 0 to 2 R^{4a1}; and
each R^{4a1} is independently halo.

15. (canceled)

16. The compound of claim 1, or the pharmaceutically acceptable salt thereof, wherein
R^{4b} is H or C₁₋₆ alkyl; and
R^{4c} and R^{4a} together with the carbon and nitrogen to which each is attached combine to form a heterocycloalkyl having 4 to 6 ring members, wherein the heterocycloalkyl is substituted with 0 to 3 R^{4a1}.

17. The compound of claim 1, or the pharmaceutically acceptable salt thereof, wherein
R^{4a} and R^{4b} are each H; and
R^{4c} is ethyl;
alternatively, R^{4c} and R^{4a} together with the carbon and nitrogen to which each is attached combine to form a pyrrolidinyl, substituted with 0 to 2 fluoro.

18. The compound of claim 1, or the pharmaceutically acceptable salt thereof, wherein
R^{5a} is H or C₁₋₆ alkyl;
R^{5b} and R^{5c} are each independently H, C₁₋₆ alkyl, —C₁₋₆ alkyl-OH, C₂₋₆ alkoxyalkyl, C₁₋₆ haloalkyl, C₃₋₆ cycloalkyl, or C₁₋₄ alkyl-C₃₋₆ cycloalkyl, wherein each cycloalkyl is substituted with 0 to 3 R^{5b5}; and
each R^{5b5} is independently C₁₋₄ alkyl, halo, or C₁₋₄ haloalkyl.

19. The compound of claim 1, or the pharmaceutically acceptable salt thereof, wherein
R^{5a} is H or C₁₋₆ alkyl;
R^{5b} is H; and
R^{5c} is C₃₋₆ cycloalkyl or C₁₋₄ alkyl-C₃₋₆ cycloalkyl.

20. The compound of claim 1, or the pharmaceutically acceptable salt thereof, wherein
R^{5a} and R^{5b} are each H; and
R^{5c} is H, methyl, ethyl,

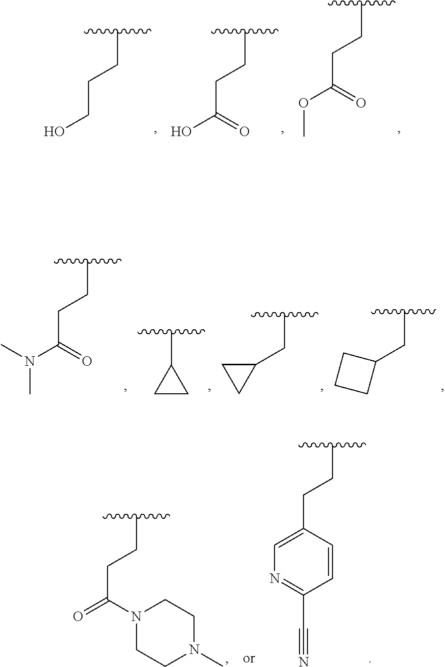

21. The compound of claim 1, or the pharmaceutically acceptable salt thereof, wherein X⁶ is C₆₋₇ alkylene.

22. The compound of claim 1, or the pharmaceutically acceptable salt thereof, wherein X⁶ is C₆₋₇ alkenylene.

23. The compound of claim 1, or the pharmaceutically acceptable salt thereof, wherein X⁶ is 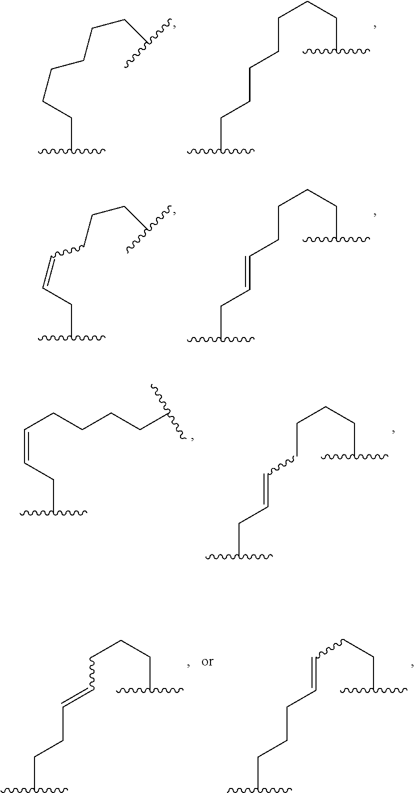
wherein the wavy bond attached to the double bond indicates E, Z, or a mixture of both isomers.

24. The compound of claim 1, or the pharmaceutically acceptable salt thereof, wherein X⁶ is 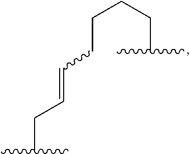 [SMILES: *CC=CCCCC*]
wherein the wavy bond attached to the double bond indicates E, Z, or a mixture of both isomers.

25. The compound of claim 1, or the pharmaceutically acceptable salt thereof, wherein X⁶ is

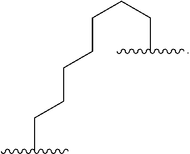 [SMILES: *CCCCCCC*]

26. The compound of claim 1, or the pharmaceutically acceptable salt thereof, wherein
R^{6a} is H, C₁₋₄ alkyl, or —C₁₋₄ alkyl-C₃₋₆ cycloalkyl; and
R^{6b} and R^{6d} are each independently H or C₁₋₆ alkyl.

27. The compound of claim 1, or the pharmaceutically acceptable salt thereof, wherein
R^{6a} is H, methyl, ethyl, or 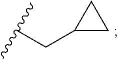 [SMILES: *CC1CC1]
R^{6b} is H; and
R^{6d} is H, methyl, or ethyl.

28. The compound of claim 1, or the pharmaceutically acceptable salt thereof, wherein
R^{7a} and R^{7b} are each independently H or C₁₋₆ alkyl; and
R^{7c} is C₁₋₆ alkyl, C₂₋₆ alkenyl, C₁₋₆ haloalkyl, C₁₋₆ alkyl-OH, or
—C₁₋₆ alkyl-C₃₋₆ cycloalkyl.

29. The compound of claim 1, or the pharmaceutically acceptable salt thereof, wherein
R^{7a} and R^{7b} are each H; and
R^{7c} is C₁₋₆ alkyl.

30. (canceled)

31. The compound of claim 1, or the pharmaceutically acceptable salt thereof, wherein
R^{7a} and R^{7b} are each H; and
R^{7c} is isobutyl.

32. The compound of claim 1, or the pharmaceutically acceptable salt thereof, wherein
R^{8a}, R^{8b}, R^{8d}, and R^{8e} are each independently H or C₁₋₆ alkyl.

33. The compound of claim 1, or the pharmaceutically acceptable salt thereof, wherein
R^{8a} is H or methyl; and
R^{8b}, R^{8d} and R^{8e} are each H.

34. The compound of claim 1, or the pharmaceutically acceptable salt thereof, wherein ring B is phenyl.

35. (canceled)

36. The compound of claim 1, or the pharmaceutically acceptable salt thereof, wherein ring B is a heteroaryl having 5 to 6 ring members and 1 to 3 heteroatoms, wherein each heteroatom is N.

37. (canceled)

38. The compound of claim 1, or the pharmaceutically acceptable salt thereof, wherein ring B is pyrid-3-yl.

39. The compound of claim 1, or the pharmaceutically acceptable salt thereof, wherein the subscript m8 is 1, 2, or 3.

40. The compound of claim 1, or the pharmaceutically acceptable salt thereof, wherein at least one R^{8f} is halo.

41. (canceled)

42. The compound of claim 1, or the pharmaceutically acceptable salt thereof, wherein
each R^{8f} is independently halo, C₃₋₆ cycloalkyl, —X^{8f}—C₃₋₆ cycloalkyl, —CH═CR^{8f1}R^{8f2}, heterocycloalkyl, —X^{8f}-heterocycloalkyl, phenyl, —X^{8f}-phenyl, heteroaryl, or —X^{8f}-heteroaryl, wherein each heterocycloalkyl has 3 to 10 ring members and 1 to 3 heteroatoms, each independently N, O, or S, and each heteroaryl has 5 to 10 ring members and 1 to 3 heteroatoms, each independently N, O, or S, wherein each cycloalkyl, heterocycloalkyl, phenyl, and heteroaryl is substituted with 0 to 3 R^{8f3};
each X^{8f} is independently C₁₋₆ alkylene, C₂₋₆ alkenylene, O, or S; and
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}.

43. The compound of claim 42, or the pharmaceutically acceptable salt thereof, wherein each X^{8f} is.

44. The compound of claim 1, or the pharmaceutically acceptable salt thereof, wherein
each R^{8f} is independently halo, C₃₋₆ cycloalkyl, —CH═CR^{8f1}R^{8f2}, heterocycloalkyl, phenyl, or heteroaryl, wherein each heterocycloalkyl has 3 to 10 ring members and 1 to 3 heteroatoms, each independently N, O, or S, and each heteroaryl has 5 to 10 ring members and 1 to 3 heteroatoms, each independently N, O, or S, wherein each cycloalkyl, heterocycloalkyl, phenyl, and heteroaryl is substituted with 0 to 3 R^{8f3};
each R^{8f1} and R^{8f2} are combined with the carbon to which they are attached to form a heterocycloalkyl having 3 to 10 ring members and 1 to 3 heteroatoms, each independently N, O or S, wherein the heterocycloalkyl is substituted with 0 to 3 R^{8f3}.

45. The compound of claim 1, or the pharmaceutically acceptable salt thereof, wherein
each R^{8f} is independently C₁₋₄ alkyl, C₂₋₄ alkenyl, C₁₋₄ alkoxy, C₁₋₄ deuteroalkoxy, halo, C₁₋₄ haloalkyl, cyano, or —C₁₋₂ alkyl-cyano.

46. (canceled)

47. The compound of claim 1, or the pharmaceutically acceptable salt thereof, wherein
each R^{8f} is independently halo, C₃₋₆ cycloalkyl, —O—C₃₋₆ cycloalkyl, heteroaryl, or —O-heteroaryl, wherein each heteroaryl has 5 to 6 ring members and 1 to 3 heteroatoms, each independently N, O, or S, wherein each cycloalkyl, and heteroaryl is substituted with 0 to 3 R^{8f3}.

48. (canceled)

49. The compound of claim 1, or the pharmaceutically acceptable salt thereof, wherein
each R^{8f3} is independently C₁₋₆ alkyl, —Y⁸—C₁₋₆ alkyl, C₁₋₆ deuteroalky,
—Y⁸—C₁₋₆ deuteroalkyl, —OH, —C₁₋₆ alkyl-OH, —Y⁸—C₁₋₆ alkyl-OH,
—C₁₋₆ alkyl-Y⁸—C₁₋₆ alkyl, halo, C₁₋₆ haloalkyl, —Y⁸—C₁₋₆ haloalkyl, or oxo;
each Y⁸ is independently C(O), C(O)O, N(R^{8f4})C(O), O, S, or S(O)₂; and
each R^{8f4} is independently H or C₁₋₆ alkyl.

50. The compound of claim 1, or the pharmaceutically acceptable salt thereof, wherein each R^{8f3} is independently C₁₋₆ alkyl, C₁₋₆ deuteroalky,
—OH, —C₁₋₆ alkyl-OH, halo, C₁₋₆ haloalkyl, or oxo.

51. The compound of claim 1, or the pharmaceutically acceptable salt thereof, wherein
each R^{8f3} is independently C₁₋₆ alkyl or —Y⁸—C₁₋₆ alkyl.

52. (canceled)

53. The compound of claim 1, or the pharmaceutically acceptable salt thereof, wherein
each R^{8f3} is independently C₃₋₆ cycloalkyl, —X^{8f3}—C₃₋₆ cycloalkyl, heterocycloalkyl, or —X^{8f3}-heterocycloalkyl, wherein each heterocycloalkyl has 3 to 6 members and 1 to 2 heteroatoms, each independently N, O, S, or S(O)₂; and
each X^{8f3} is independently C₁₋₆ alkylene, C(O), or S(O)₂.

54.-57. (canceled)

58. The compound of claim 1, or the pharmaceutically acceptable salt thereof, wherein
m8 is 1, 2, or 3; and
each R^{8f} is independently methyl, isopropyl, isobutyl, methoxy, ethoxy, fluoro, chloro, cyano, 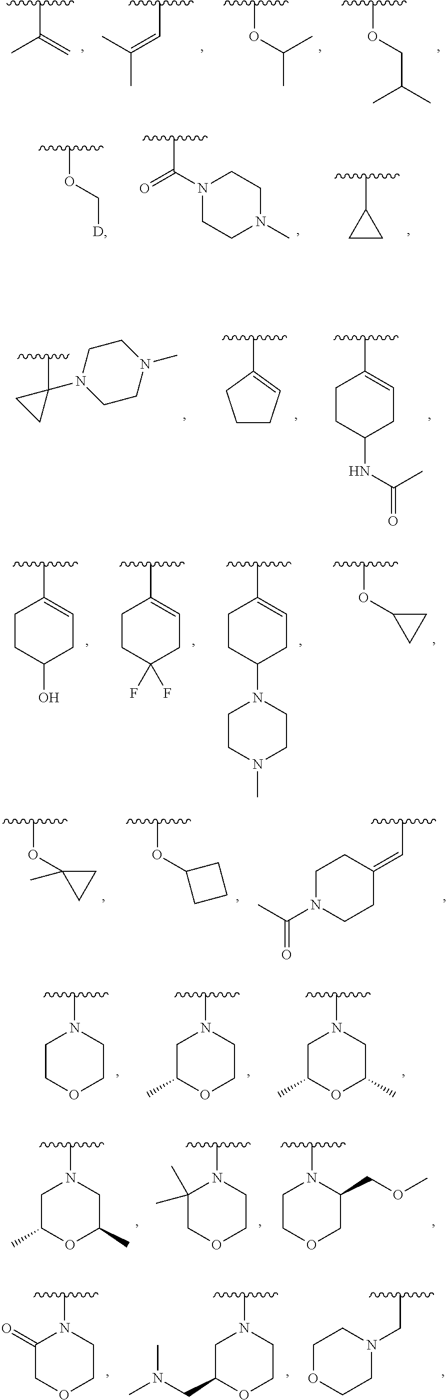 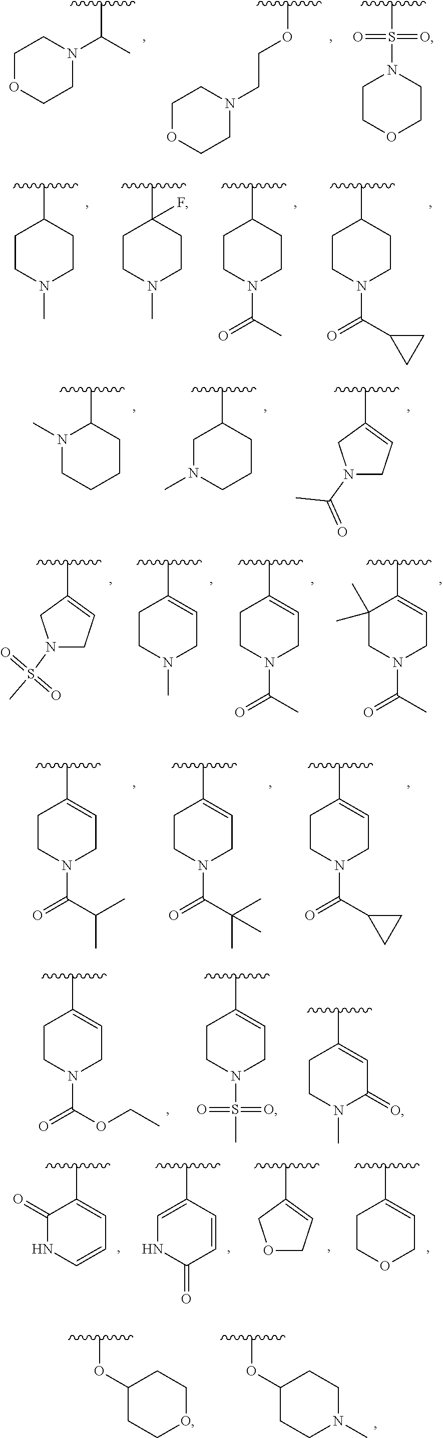 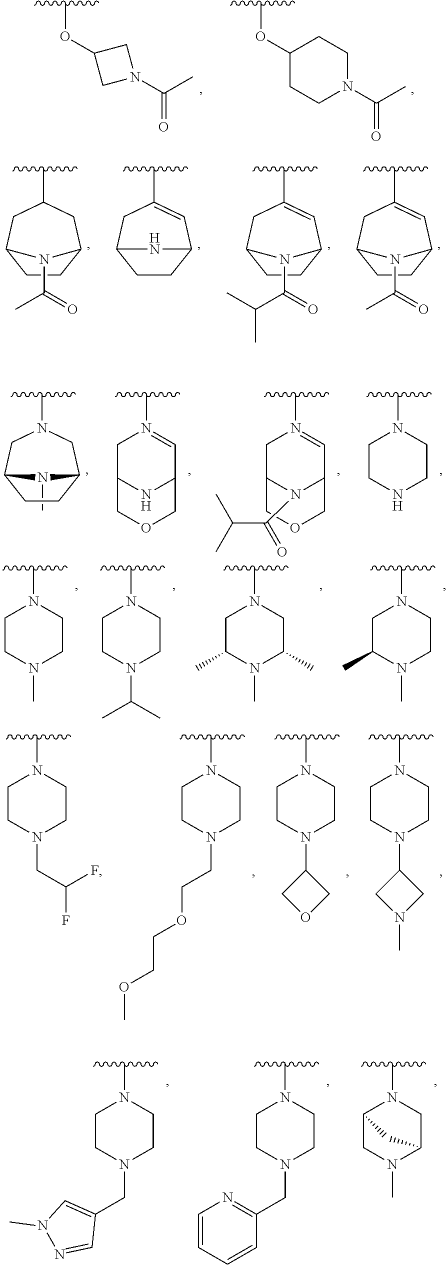 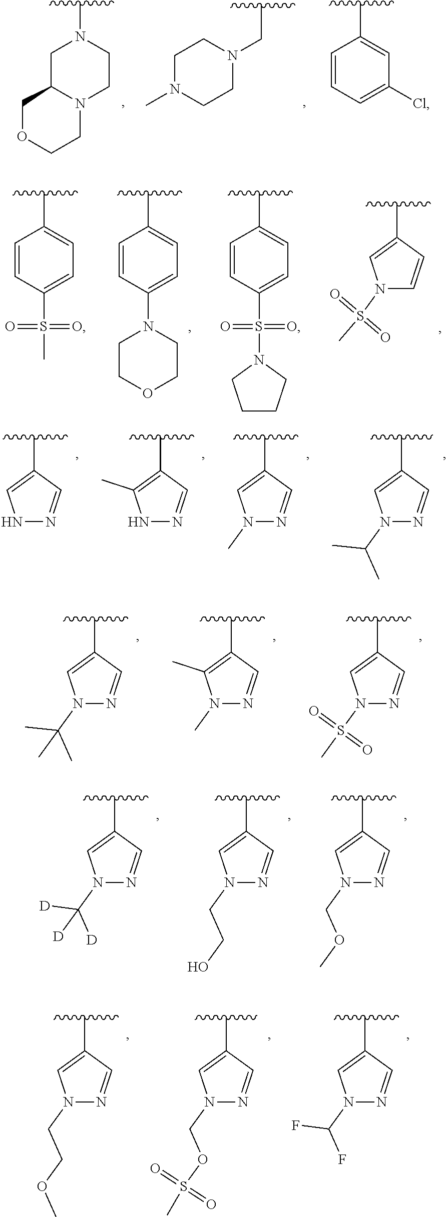 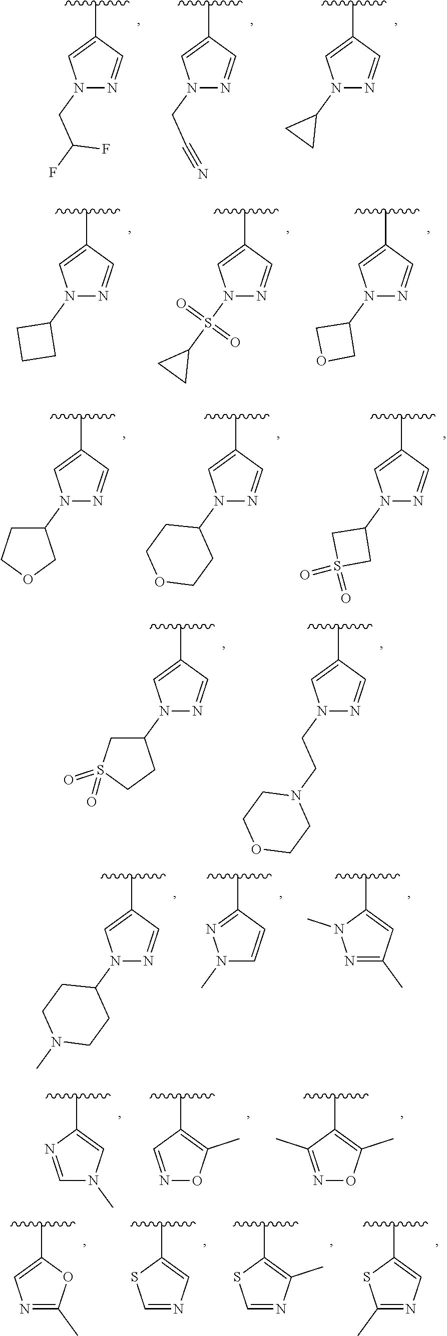 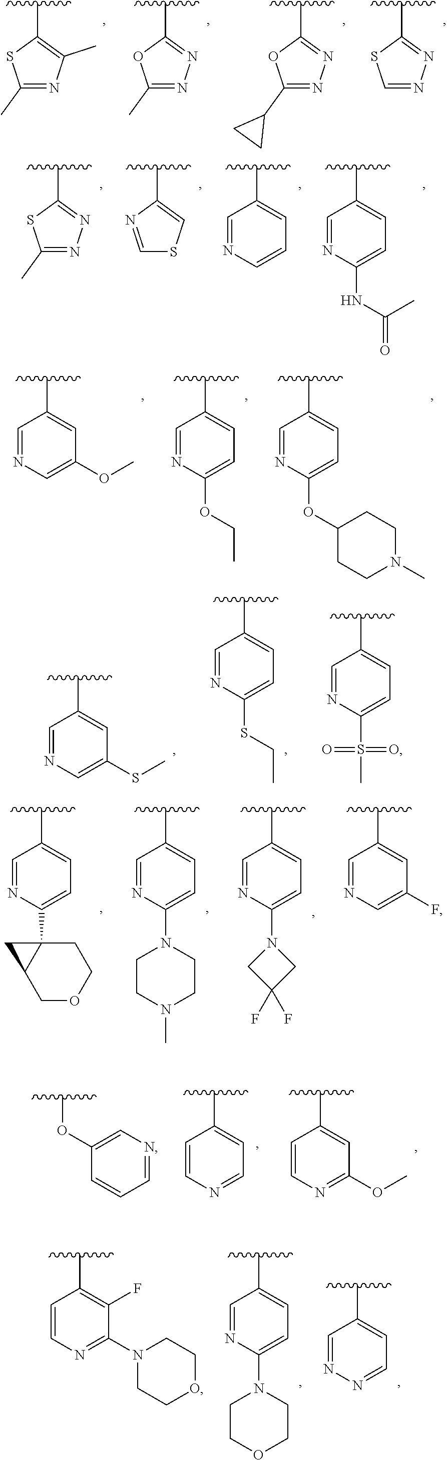 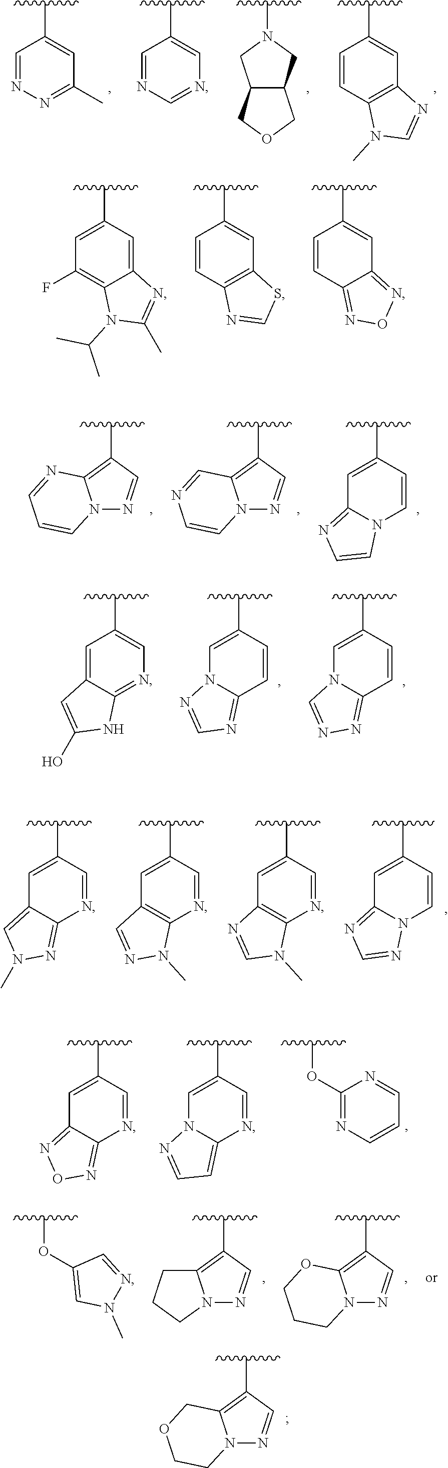
alternatively, ring B and three R^{8f} groups form the moiety 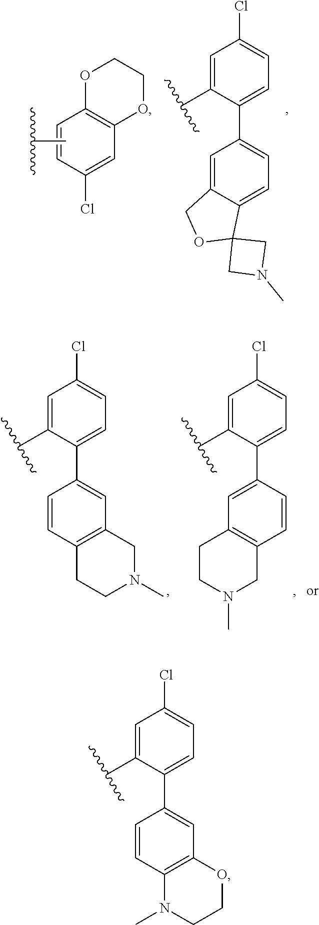
wherein the wavy line identifies attachment to the remainder of the molecule.

59. The compound of claim 1, or the pharmaceutically acceptable salt thereof, wherein
R³ is 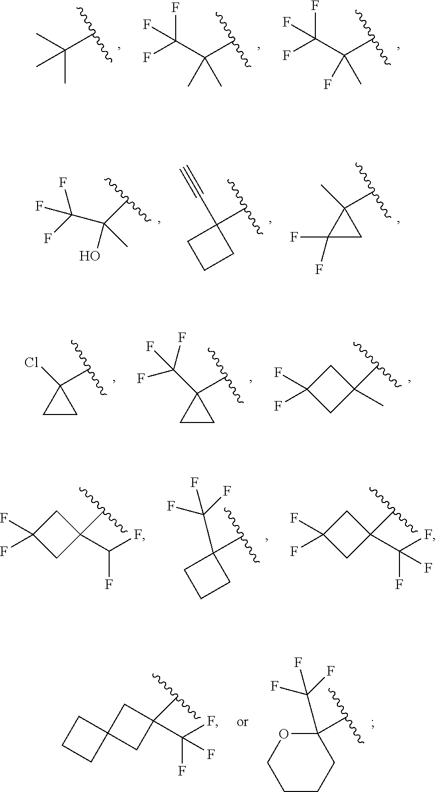
R^{4a} and R^{4b} are each H;
R^{4c} is ethyl;
alternatively, R^{4c} and R^{4a} together with the carbon and nitrogen to which each is attached combine to form a pyrrolidinyl, substituted with 0 to 2 fluoro;
R^{5a} and R^{5b} are each H;
R^{5c} is H, methyl, ethyl, 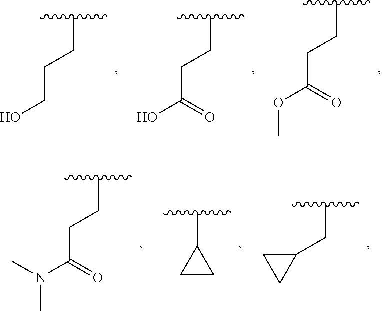 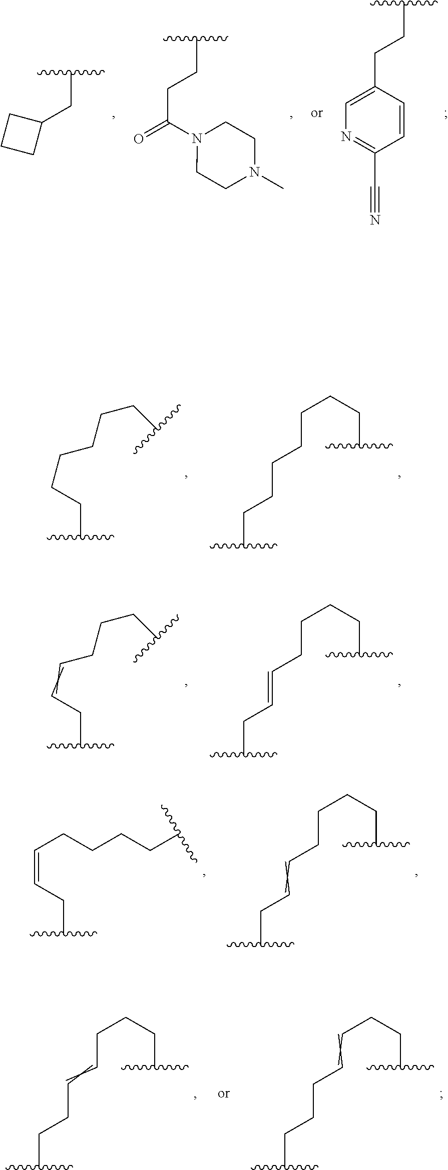
X⁶ is
R^{6a} is H, methyl, ethyl, 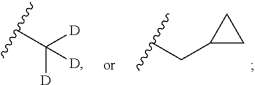
R^{6b} is H;
R^{6d} is H, methyl, or ethyl;
R^{7a} and R^{7b} are each H;
R^{7c} is isobutyl, 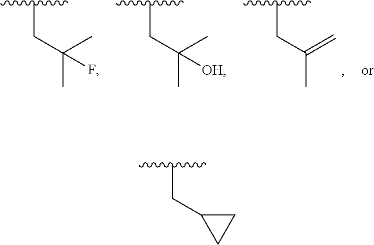
R^{8a} is H or methyl;
R^{8b}, R^{8d} and R^{8e} are each H;
ring B is phenyl, naphthyl, biphenyl, thiophenyl, pyridyl, benzofuranyl, indolyl, indazolyl, or quinolinyl;
m8 is 1, 2, or 3; and
each R^{8f} is independently methyl, isopropyl, isobutyl, methoxy, ethoxy, fluoro, chloro, cyano, 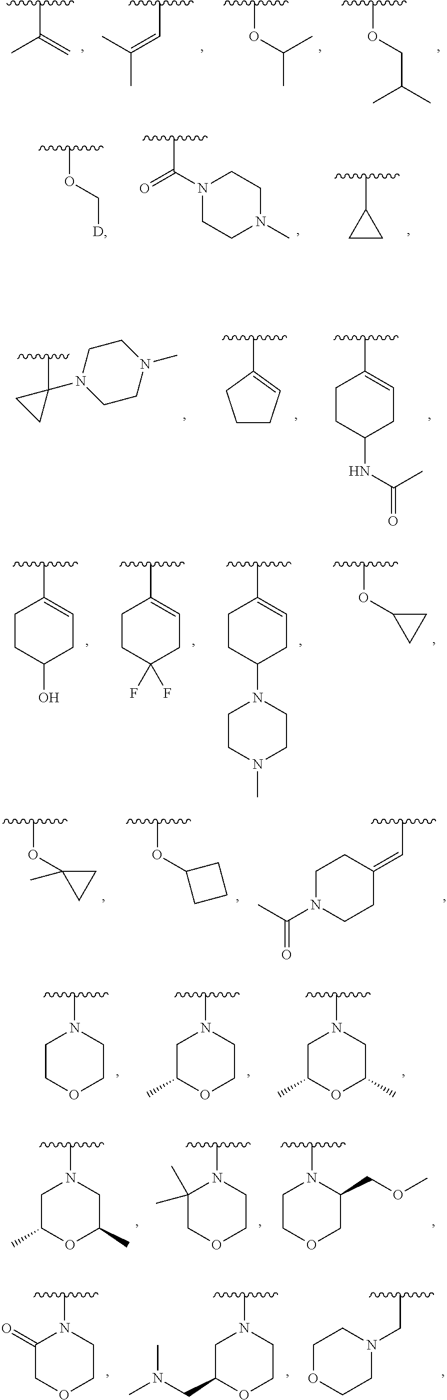 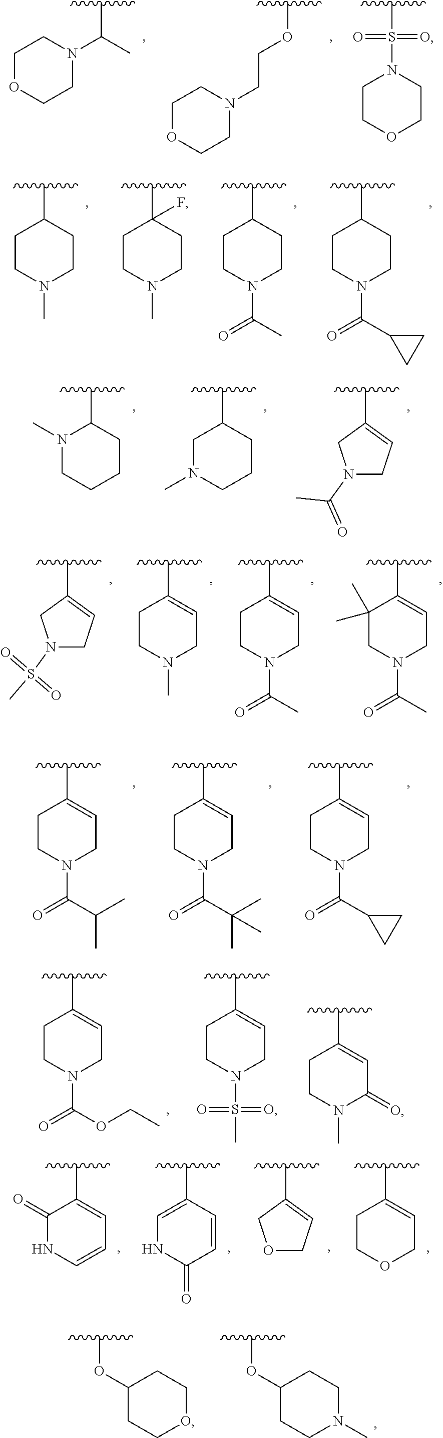 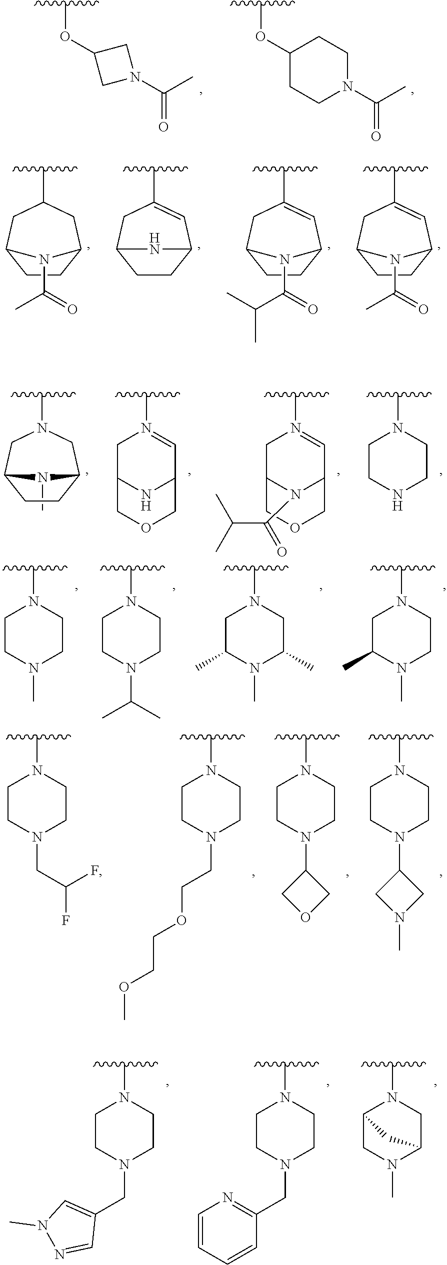 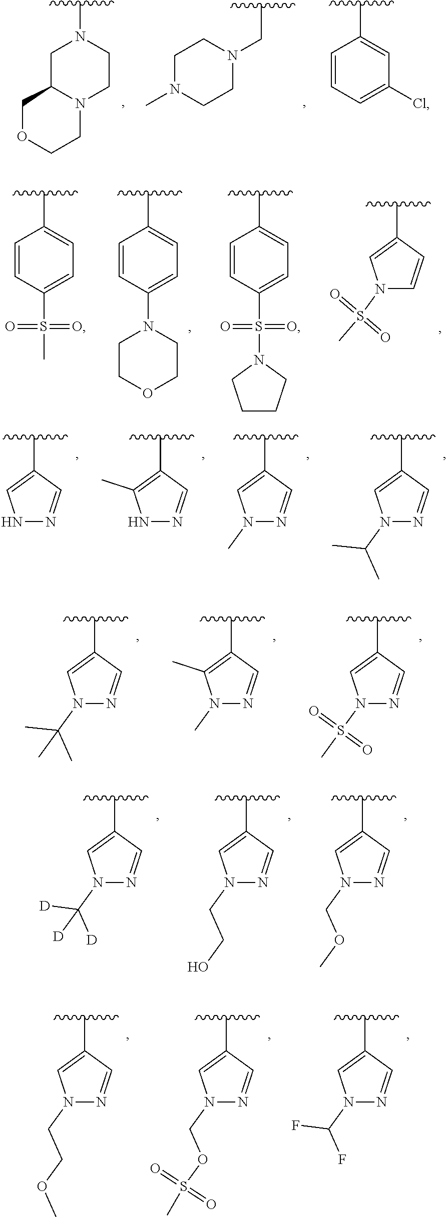 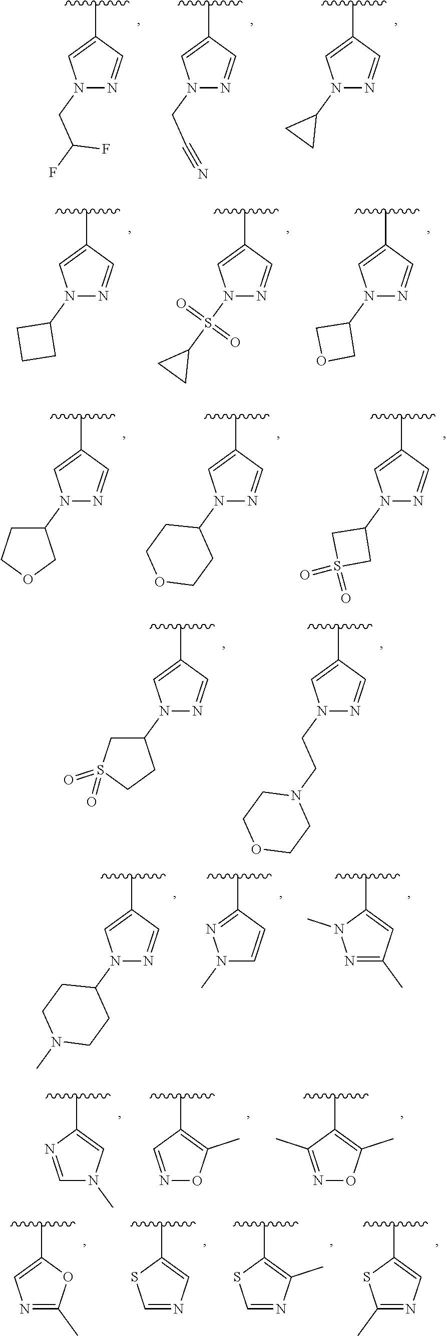 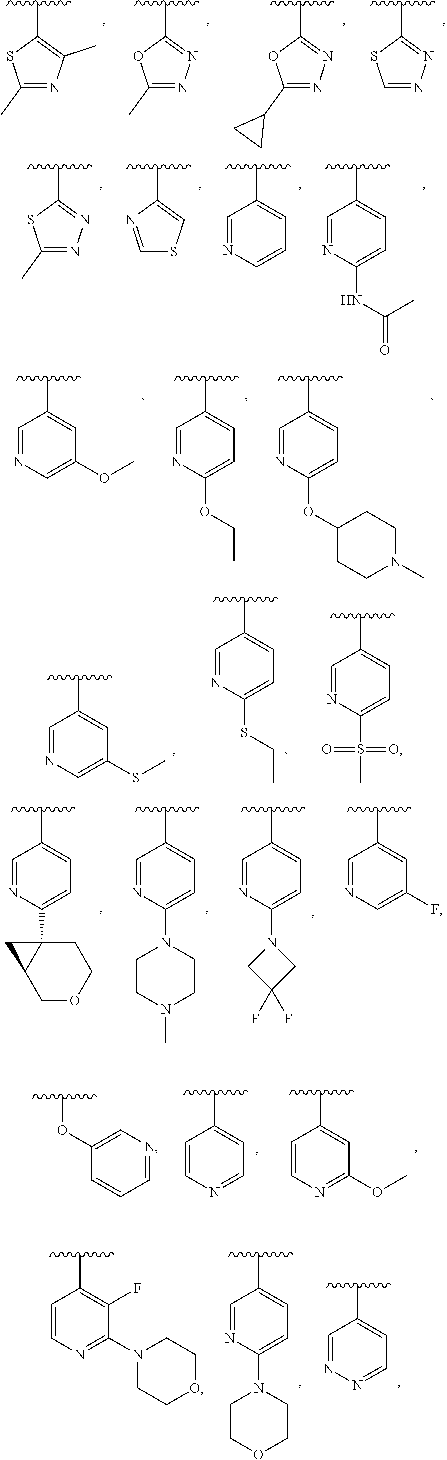 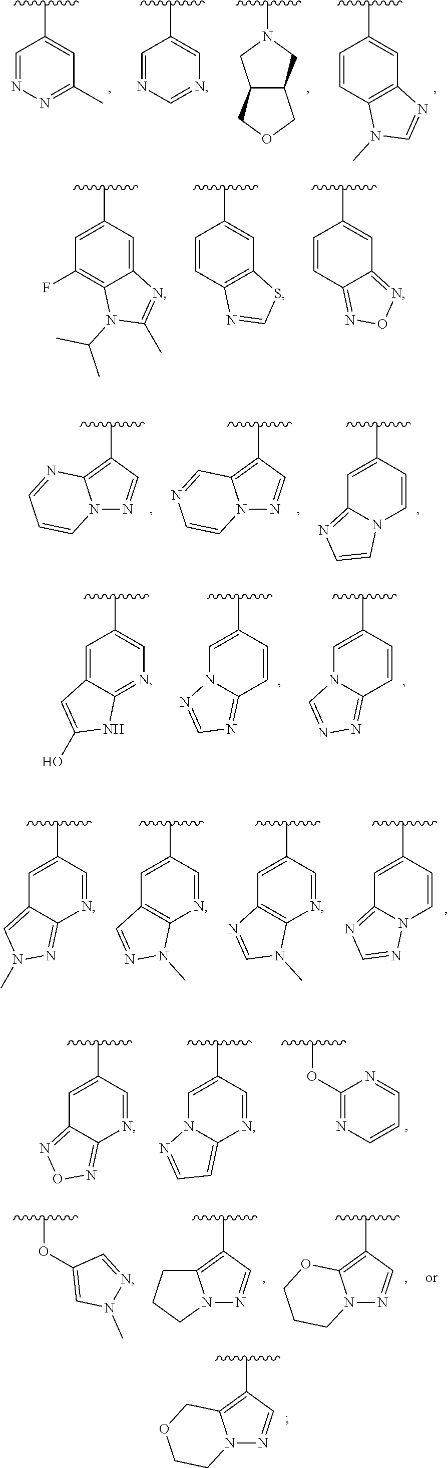
alternatively, ring B and three R^{8f} groups form the moiety 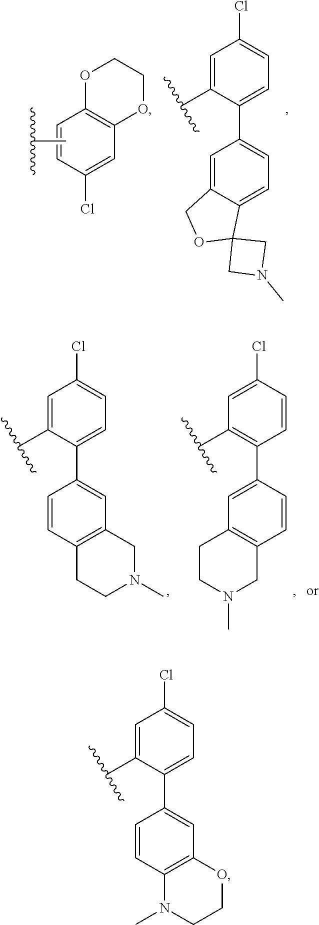
wherein the wavy line identifies attachment to the remainder of the molecule.

60. (canceled)

61. The compound of claim 1, or the pharmaceutically acceptable salt thereof, having Formula (Ic):

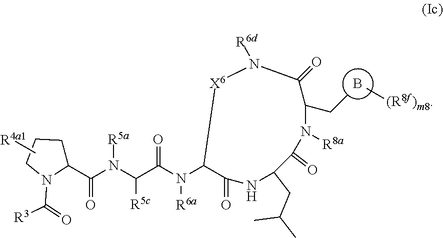

62. (canceled)

63. The compound of claim 1, or the pharmaceutically acceptable salt thereof, having the structure of any one of Examples 1-426.

64. The compound of claim 1, or the pharmaceutically acceptable salt thereof, having the structure

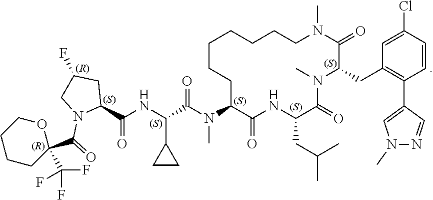

65. The compound of claim 1, or the pharmaceutically acceptable salt thereof, having the structure

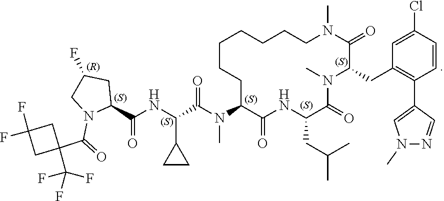

66. The compound of claim 1, or the pharmaceutically acceptable salt thereof, having the structure

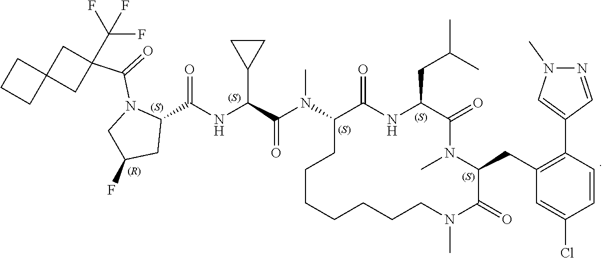

67. The compound of claim 1, or the pharmaceutically acceptable salt thereof, having the structure

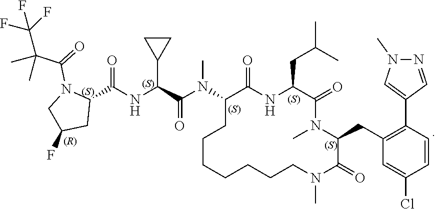

68. The compound of claim 1, or the pharmaceutically acceptable salt thereof, having the structure

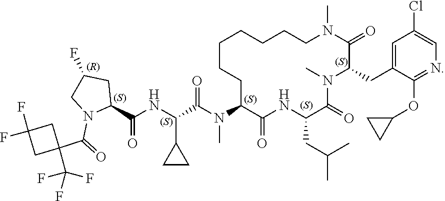

69. The compound of claim 1, or the pharmaceutically acceptable salt thereof, having the structure

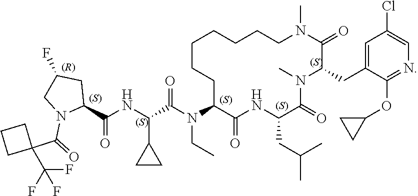

70. The compound of claim 1, or the pharmaceutically acceptable salt thereof, having the structure

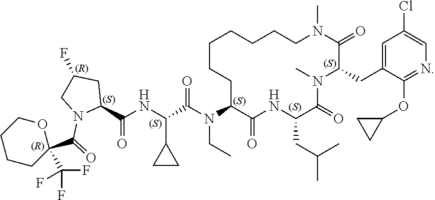

71. The compound of claim 1, or the pharmaceutically acceptable salt thereof, having the structure

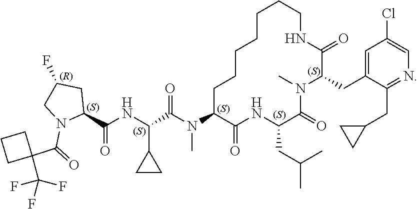

72. A pharmaceutical composition comprising a compound of claim 1 and a pharmaceutically acceptable excipient.

73. A method of treating a cancer mediated at least in part by cyclin A comprising administering a therapeutically effective amount of a compound of claim 1, thereby treating the disorder or condition.

74.-75. (canceled)
